(19) United States
(12) Patent Application Publication
(10) Pub. No.: US 2025/0257076 A1
(43) Pub. Date: Aug. 14, 2025

(54) SUBSTITUTED PYRROLIDINE-2-CARBOXYLIC ACID DERIVATIVES AS cGAS INHIBITORS

(71) Applicant: Janssen Pharmaceutica NV, Beerse (BE)

(72) Inventors: Stephen MUPRHY, San Diego, CA (US); Kelly McCLURE, Ramona, CA (US); Yuri LEE, Irvine, CA (US); David SCHUMAN, Carlsbad, CA (US)

(21) Appl. No.: 19/052,542

(22) Filed: Feb. 13, 2025

Related U.S. Application Data
(60) Provisional application No. 63/553,382, filed on Feb. 14, 2024.

(51) Int. Cl. C07D 519/00; A61K 31/5386

(52) U.S. Cl. CPC C07D 519/00; A61K 31/5386

(57) ABSTRACT

Compounds of Formula (II), pharmaceutical compositions containing them, methods of making them, and methods of using them including methods for treating disease states, disorders, and conditions associated with the cGAS pathway, such as autoimmune disorders including Aicardi-Goutieres Syndrome (AGS), Systemic Lupus Erythematosus (SLE), Lupus Nephritis, Scleroderma, Sjogren's Syndrome, Inflammatory Myopathies, Hidradenitis Supperativa (HS), Parkinson's Disease, Rheumatoid Arthritis, Ulcerative Colitis and Crohn's Disease,

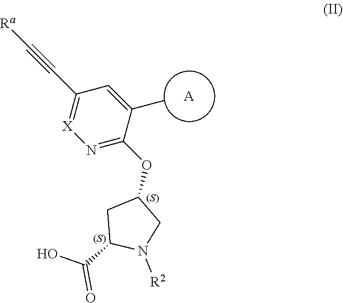

wherein R^{a}, \textcircled{A}, R¹ and R², are defined herein.

### CROSS-REFERENCE TO RELATED APPLICATIONS

This application claims the benefit under 35 U.S.C. § 119(e) of U.S. Provisional Application No. 63/553,382, filed Feb. 14, 2024, the entire disclosure of which is hereby incorporated herein by reference.

### FIELD

The present disclosure is directed towards pyrrolidine-2-carboxylic acid derivatives, stereoisomers, and pharmaceutically acceptable salts thereof, pharmaceutical compositions containing said compounds, and the use of said compounds in the treatment of autoimmune disorders including Aicardi-Goutieres Syndrome (AGS), Systemic Lupus Erythematosus (SLE), Lupus Nephritis, Systemic Sclerosis, Sjogren's Syndrome, Inflammatory Myopathies, Hidradenitis Suppurativa, Parkinson's Disease, Rheumatoid Arthritis, Ulcerative Colitis and Crohn's Disease.

### BACKGROUND

Pattern Recognition Receptors (PRR) are one of the fundamental mechanisms of how the Myeloid cells of the immune system monitor their environment for disturbances to homeostasis. The presence of double stranded DNA in the cytoplasm of eukaryotic cells acts as a danger signal to trigger the host immune response. Cyclic GMP-AMP synthase (cGAS) is a 59 kDa nucleotidyl transferase which acts as a PRR for double stranded DNA (Ablasser et al, cGAS produces a 2′-5′-linked cyclic dinucleotide second messenger that activates STING. Nature 498, 380-384; 20133; Wu et al, cyclic GMP-AMP Is an endogenous second messenger in innate immune signaling by cytosolic DNA. Science 330 826-830, 2013; Sun et al, cyclic GMP-AMP Synthase Is a cytosolic DNA Sensor that activates the Type 1 Interferon Pathway. Science 339, 786-791, 2013). Upon DNA binding, cGAS catalyzes the cyclization of ATP and GTP to form the secondary signaling molecule, 2′3′cGAMP or cGAMP. cGAMP in-turn is a natural ligand for STING (Stimulator of Interferon Genes) which through Tank-binding Kinase 1(TBK1)/Interferon Regulatory Factor 3 (IRF3) induces interferon response factors (IRF) and nuclear factor kappa-light-chain-enhancer of activated B cells (NFkB) driven gene expression.

Deoxyribonuclease (Dnase) deficiencies result in excess DNA and have been linked to aberrant DNA sensing and subsequent increased pathological anti-nuclear antibody titers in human SLE patients (Yasutomo et al., Mutation of DNASE1 in people with systemic lupus erythematosus. Nature Genetics 28:313, 2001). This phenomenon has been recapitulated in a mouse model of DNASE deficiency via the three-prime repair exonuclease 1 (TREX1) mutant mouse model. TREX1 acts as part of the SET complex to degrade DNA. The TREX1 model manifests as an autoimmune phenotype with multi-organ inflammation and increased interferon signature gene (ISG) expression similar to SLE. However, if these mice are bred on to a cGAS deficient background, the animals are protected from disease as reported by Gao et al (Gao et al., Activation of cyclic GMP-AMP synthase by self-DNA causes autoimmune diseases. Proceedings of the National Academy of Sciences 112:E5699-E5705, 2015). And critically, the authors report anti-dsDNA antibodies are reduced in the cGAS/TREX1 double knockouts, which highlights the role cGAS plays in DNA-driven immune mediated pathology. Similarly, Aicardi-Goutieres syndrome or AGS is a lupus-like autoinflammatory disorder that results from mutations in the DNA exonuclease, TREX1 (Crow et al, Mutations in the gene encoding the 3′-5′ DNA exonuclease TREX1 Aicardi-Goutieres syndrome at the AGS1 locus, Nature Genetics, 38:917-920, 2006). These observations support a role for the inhibition of cGAS as a therapeutic strategy for treating SLE/Lupus Nephritis and AGS. Additionally, emerging evidence has linked aberrant DNA exposure in the form of cell free DNA with reduced TREX expression in Rheumatoid Arthritis Patients (Luo et al, Age-related self-DNA accumulation may accelerate arthritis in rats and in human rheumatoid arthritis. Nature Communications, 20; 14(1):4394, 2023) thereby supporting a role for inhibition of cGAS in Rheumatoid Arthritis. A similar link to aberrant DNA exposure in the form of Neutrophil Extracellular Traps (NETS) has been linked to cGAS in manifestations of Hidradenitis Suppurativa (Byrd et al, Neutrophil extracellular traps, B cells, and type I interferons contribute to immune dysregulation in hidradenitis suppurativa. Sci Transl Med., 508: 2019). Additionally, a recent study by Papinksa and colleagues (Papinska et al, Activation of Stimulator of Interferon Genes (STING) and Sjogren Syndrome. Journal of Dental Research, 97, 893-900, 2018) demonstrated that activating the cGAS pathway through activation of STING with a STING agonist induced Sjogren syndrome like disease in mice, thus supporting a role for inhibition of the upstream activator of STING as a therapeutic strategy for Sjogren's syndrome.

Similarly, systemic sclerosis has been linked to the cGAS-STING pathway thorough chromosomal instability as evident by the recent work by Paul et al (Paul et al, Centromere defects, chromosome instability, and cGAS-STING activation in systemic sclerosis Nature Communications, 13, 7074, 2022). Similarly, a role for the cGAS pathway in driving inflammatory myopathies was demonstrated by the Zhou and colleagues (Zhou et al, Activation of cGAS-STING pathway—A possible cause of myofiber atrophy/necrosis in dermatomyositis and immune-mediated necrotizing myopathy, Journal of Clinical Laboratory Analysis, 36, 24631, 2022). Finally, a role for the cGAS pathway was recently demonstrated for Parkinson's disease wherein mouse models highlighted microglial driven inflammation (Ma et al, Microglial cGAS drives Neuroinflammation in the MPTP Mouse Model of Parkinson's Disease, CNS Neuroscience Therapy, 29, 2018-2035, 2023). Targeting the cGAS pathway is therefore a possible strategy for the treatment of autoimmune diseases.

Given the potential role of the cGAS-STING pathway in autoimmune diseases, efforts to develop small molecule cGAS inhibitors have been explored by the scientific community. For example, WO 2019/241787 describes methyl 4-amino-6-(phenylamino)-1,3,5-triazine-2-carboxylates as cGas inhibitors. U.S. Pat. No. 12,043,625 and US Pat. Application Publication No. 2023/0000878 also describe small molecule cGas inhibitors. For example, U.S. Pat. No. 12,043,625 describes pyridine derivatives with C-linked cyclic substitutes as cGAS inhibitors and US Pat. App. Pub. No. 2023/0000878 describes pyridine derivatives with N-linked cyclic substitutes as cGAS inhibitors (see e.g., Table 23 herein, entries 1-4 for representative example compounds).

Despite the efforts to develop small molecule cGAS inhibitors, their development has remained challenging. For example, no small molecule cGAS inhibitor has been approved and none have entered late-stage clinical trials. As of Jan. 22, 2025, publicly available information shows that only three cGAS inhibitors have entered phase I clinical trials, which include: a phase 1 study of VENT-03 (EUCT number:2023-507504-31-00) completed October 2024, a phase 1 study for GSK4347859 (NCT06188507) that started Q1 2024 with a completion date projected in 2026, and a phase 1 study of IMSB301 (ISRCTN90049550) initiated in Q2 2024 with a planned completion date in Q2 2025.

Accordingly, there is a long-felt and unmet need for the development of a small molecule cGAS inhibitor as their development may provide and/or broaden treatment options for patients with an autoimmune disorder (e.g., Aicardi-Goutieres Syndrome (AGS), Systemic Lupus Erythematosus (SLE), Lupus Nephritis, Systemic Sclerosis, Sjogren's Syndrome (SjD), Inflammatory Myopathies, Hidradenitis Suppurativa, Parkinson's Disease, Rheumatoid Arthritis, Ulcerative Colitis and Crohn's Disease).

### BRIEF SUMMARY

Surprisingly, it has been found that compounds of Formula (I) and Formula (II) are potent inhibitors of cGAS with IC₅₀ values ranging from 0.0003 μM to 0.4631 μM with regards to inhibiting the production of IP-10 in cells stimulated with double-stranded DNA. See e.g., Assay No. 1 described in Table 22. Given the higher order alkynyl groups in compounds of Formula (I) and Formula (II), it was unexpected that these compounds would bind to the cGAS target—let alone inhibit cGAS with the high potency observed (see, e.g., FIGS. 1-3). Moreover, compounds of Formula (IIB) maintain their high potency while also exhibiting increased permeability, hepatocyte stability, and/or bioavailability as compared to representative examples from US Pat. App. Pub. No. 2023/0000878 (see, e.g., Table 23).

In some embodiments, disclosed herein are compounds of Formula (I),

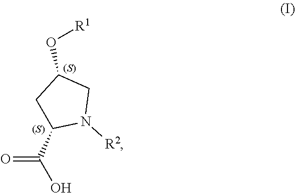

and pharmaceutically acceptable salts or stereoisomers thereof,
wherein:
R¹ is selected from:

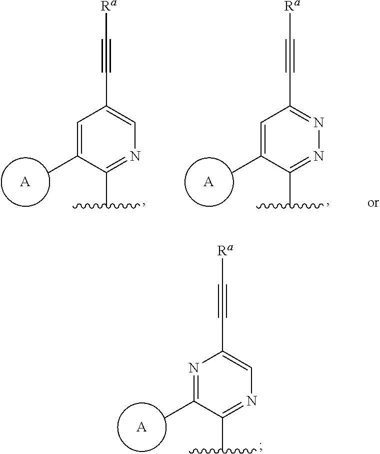

wherein \textcircled{A} is a nitrogen linked monocyclic heterocycloalkyl, spiro-heterocycloalkyl, or bridged heterocycloalkyl selected from the group consisting of:

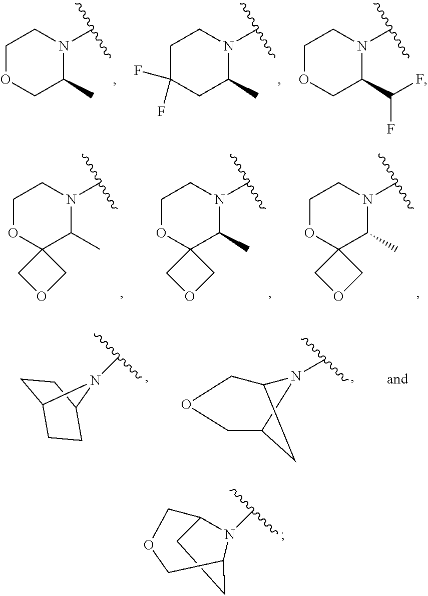

wherein R^{a} is selected from the group consisting of:
   (i) —C₁₋₆alkyl optionally substituted with one, two, or three members each independently selected from the group consisting of: —F, —OH, —OC₁₋₆alkyl, —NH(SO₂CH₃), —OCH₂CH₂OCH₃, and —N(C₁₋₄alkyl)₂; or
   (ii) —C₃₋₆cycloalkyl or -L¹-C₃₋₆cycloalkyl, wherein the cycloalkyl is monocyclic, spirocyclic, bridged or fused, optionally substituted with one, two, or three members each independently selected from the group consisting of: —F, —C₁₋₆alkyl, —C₁₋₆haloalkyl, —OC₁₋₆alkyl, and 1H-pyrazole; wherein -L¹- is —C(H)(OCH₃)— or —C₁₋₃alkyl; or
   (iii) phenyl or -L²-phenyl, wherein the phenyl is optionally substituted with one, two, or three members each independently selected from the group consisting of: —F, —C₁₋₆alkyl, —C₁₋₆haloalkyl, —OC₁₋₆alkyl, —N(C₁₋₄alkyl)₂, and 4-methyl-1H-imidazole; wherein -L²- is —CH₂O—; or
   (iv) heterocycloalkyl or —La-heterocycloalkyl; wherein the heterocycloalkyl is selected from the group consisting of:

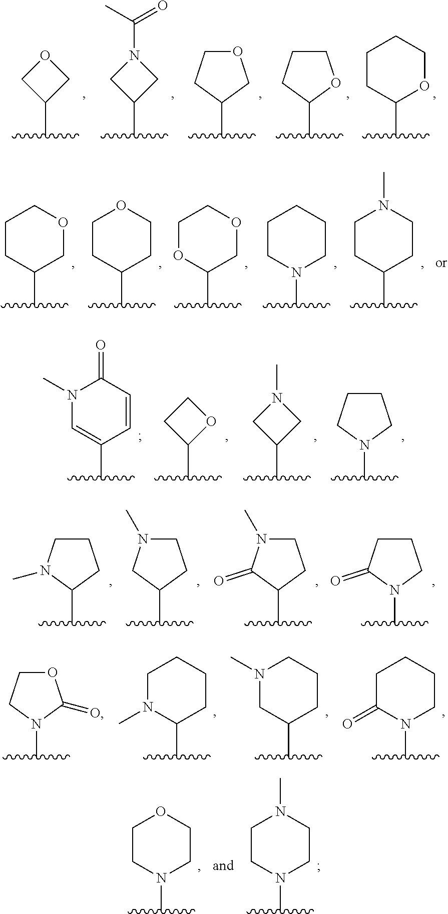

wherein each heterocycloalkyl is optionally substituted with one, two, or three members each independently selected from: —F, —OH, —C₁₋₆alkyl, —C₁₋₆haloalkyl, and —N(C₁₋₄alkyl)₂; wherein -L₃- is —C₁₋₃alkyl-, or —CH₂O—; or
(v) 5-membered heteroaryl or -L³-(5-membered heteroaryl), wherein the 5-membered heteroaryl is selected from the group consisting of:

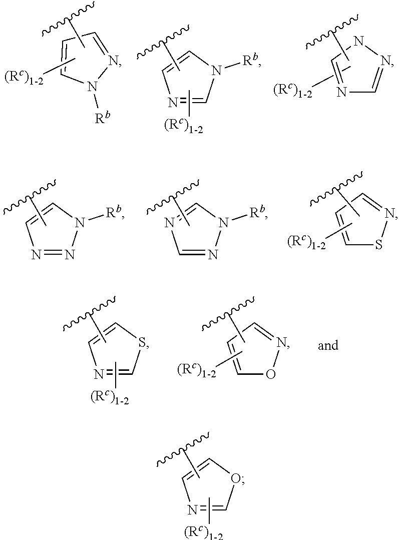

wherein R^{b} is H, or —C₁₋₆alkyl; each R^{c} is independently selected from the group consisting of: H, —C₁₋₆alkyl, —C₁₋₆haloalkyl, —OC₁₋₆alkyl, —CH₂OCH₃, and cyclopropyl; wherein -L³- is —C₁₋₃alkyl-, or —CH₂O—; or
   (vi) 6-membered heteroaryl or -L⁴-(6-membered heteroaryl); wherein the 6-membered heteroaryl is selected from the group consisting of:

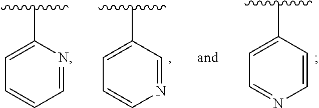

wherein each 6-membered heteroaryl is optionally substituted with one, two, or three members each independently selected from the group consisting of: —F, —C₁₋₆alkyl, —C₁₋₆haloalkyl, —OC₁₋₆haloalkyl, and —OC₁₋₆alkyl; wherein -L⁴- is —C₁₋₃alkyl-, or —CH₂O—; or
   (vii) 5,6-fused bicyclic heteroaryl or 6,6-fused bicyclic heteroaryl selected from the group consisting of:

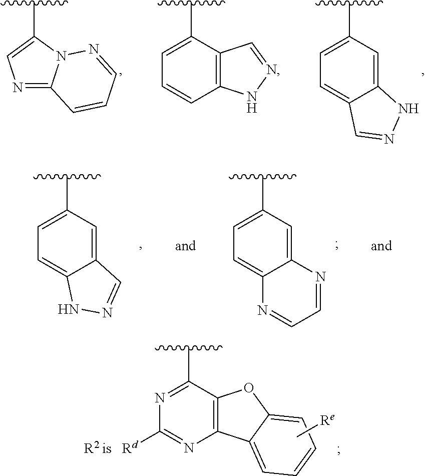

wherein
R^{d} is selected from the group consisting of: —C₁₋₆alkyl; —C₁₋₆haloalkyl; —OC₁₋₆alkyl;
—C₃₋₆cycloalkyl; —N(C₁₋₄alkyl)₂;
-heterocycloalkyl optionally substituted with one or two substituents each independently selected from halo, and —C₁₋₆alkyl; and

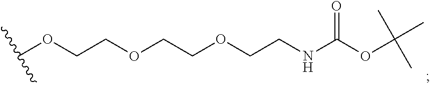 [SMILES: *OCCOCCOCCNC(=O)OC(C)(C)C]

and
R^{e} is selected from the group consisting of: H, F, —C₁₋₆alkyl, and —C₁₋₆haloalkyl.

In some embodiments, disclosed herein are compounds of Formula (II), or a pharmaceutically acceptable salt thereof,

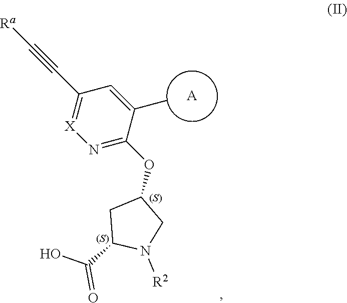

wherein:
\textcircled{A} is

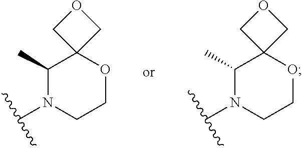 [SMILES: *N1CCOC2(COC2)[C@@H]1C ; *N1CCOC2(COC2)[C@H]1C]

X is CH or N;
R^{a} is selected from the group consisting of:
   (i) —C₁₋₆alkyl substituted with one or two members each independently selected from the group consisting of: —OH, and —OC₁₋₆alkyl; or
   (ii) —C₃₋₆cycloalkyl or -L¹-C₃₋₆cycloalkyl, wherein the cycloalkyl is monocyclic or fused bicyclic, and unsubstituted or substituted with one or two members each independently selected from the group consisting of: —F, —OH, —OC₁₋₆alkyl, and 1H-pyrazole; wherein -L¹- is —C₁₋₃alkyl; or
   (iii) phenyl or -L²-phenyl, wherein the phenyl is unsubstituted or substituted with —C₁₋₆alkyl, or —OC₁₋₆alkyl; wherein -L²- is —CH₂O—; or
   (iv) heterocycloalkyl or -L³-heterocycloalkyl; wherein the heterocycloalkyl is selected from the group consisting of:

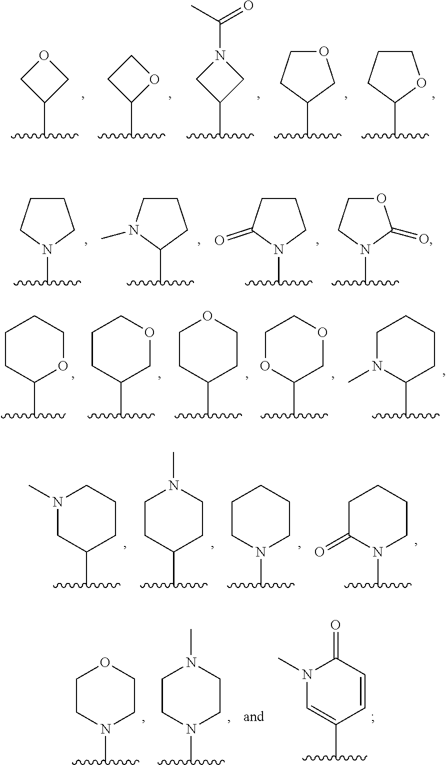

wherein each heterocycloalkyl is unsubstituted or substituted with one or two members each independently selected from: —F, —OH, —CH₂OH, and —C₁₋₆alkyl; wherein -L₃- is —C₁₋₃alkyl-; or
(v) 5-membered heteroaryl or -L³-(5-membered heteroaryl), wherein the 5-membered heteroaryl is selected from the group consisting of:

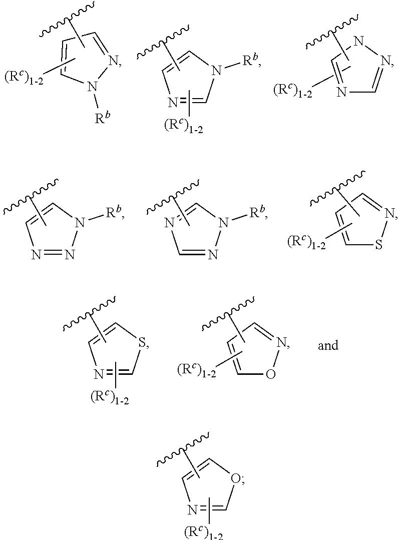

wherein R^{b} is —C₁₋₆alkyl or cyclopropyl; each R^{c} is independently selected from the group consisting of: H, —C₁₋₆alkyl, —OC₁-alkyl, and cyclopropyl; wherein -L³- is —C₁₋₃alkyl; or
(vi) 6-membered heteroaryl; wherein the 6-membered heteroaryl is selected from the group consisting of:

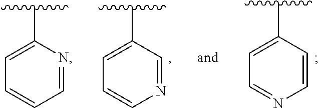

wherein each 6-membered heteroaryl is unsubstituted or substituted with one or two members each independently selected from the group consisting of: —F, —C₁₋₃alkyl, —C₁₋₃haloalkyl, and —OC₁₋₆alkyl; or
(vii)

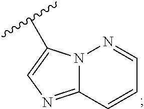 [SMILES: *c1cnc2cccnn12]

and
R² is

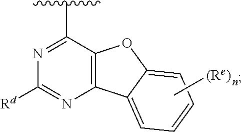

wherein
R^{d} is selected from the group consisting of: -C₁₋₆alkyl; —C₁₋₆haloalkyl; —OC₁₋₆alkyl; -cyclopropyl; heterocycloalkyl, wherein the heterocycloalkyl- is selected from the group consisting of:

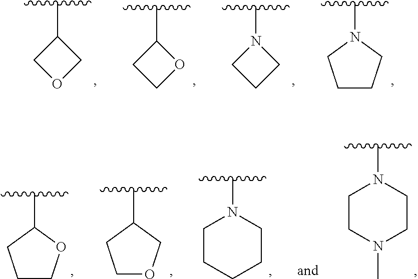

wherein each heterocycloalkyl is unsubstituted or substituted with one or two substituents each independently selected from halo, and -C₁₋₆alkyl; and

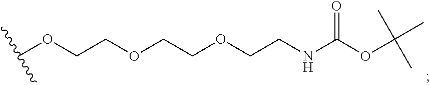 [SMILES: *OCCOCCOCCNC(=O)OC(C)(C)C]

R^{e} is selected from the group consisting of: —F, —C₁₋₃alkyl, and —C₁₋₃haloalkyl; and
n is 0, 1, or 2.

In some embodiments, disclosed herein is a compound of Formula (IIB), or a pharmaceutically acceptable salt thereof,

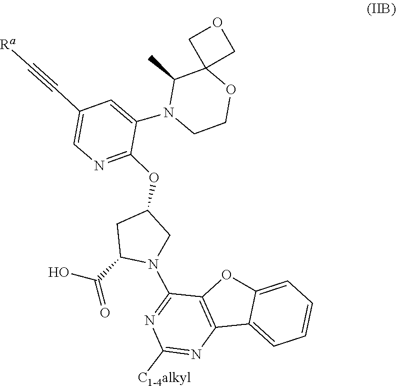

wherein
R^{a} is
   (i) 5-membered heteroaryl; wherein the 5-membered heteroaryl is selected from the group consisting of:

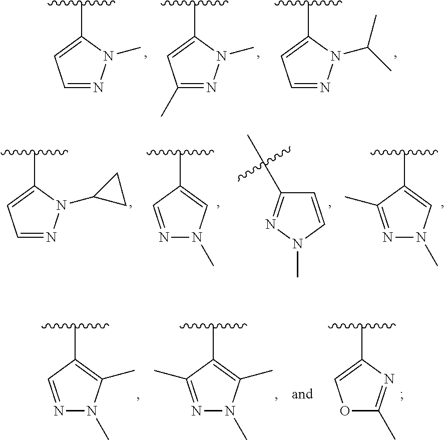

(ii) 6-membered heteroaryl; wherein the 6-membered heteroaryl is selected from the group consisting of:

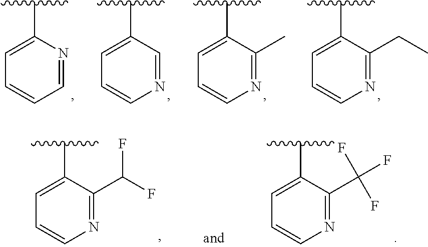

As described herein, embodiments of the present disclosure relate to chemical entities, pharmaceutical compositions containing them, methods of making and purifying them, and methods for using them in the treatment of diseases, disorders, and conditions associated with cGAS inhibition. An additional embodiment of the present disclosure is a method of treating a subject suffering from or diagnosed with a disease, disorder, or condition associated with cGAS inhibition using at least one chemical entity of the present disclosure.

Additional embodiments, features, and advantages of the present disclosure will be apparent from the following detailed description and through practice of the present disclosure.

### BRIEF DESCRIPTION OF THE DRAWINGS

FIG. 1 is the cGAS-DNA bound structure from W. Xie et al., Human cGAS catalytic domain has an additional DNA-binding interface that enhances enzymatic activity and liquid-phase condensation, Proc. Natl. Acad. Sci. U.S.A. 116 (24) 11946-11955, https//doi.org/10.1073/pnas.1905013116 (2019).

FIG. 2 is the cGAS-DNA bound structure with Example 1.11 from US 2023/000078; here, Ex. 1.11 is in an energetically minimized conformation in water, then inserted in the cGAS receptor using substructural overlay with a literature structure from US 2022/0073532 (e.g., FIG. 4 in US 2022/0073532).

FIG. 3 is the cGAS-DNA bound structure with Example 36 from the instant disclosure; here, Example 36 is in an energetically minimized conformation in water, then inserted in the cGAS receptor using substructural overlay with a literature structure from US 2022/0073532 (e.g., FIG. 4 in US 2022/0073532).

### DETAILED DESCRIPTION

Unless defined otherwise, all technical and scientific terms used herein have the same meaning as commonly understood to one of ordinary skill in the art to which this disclosure pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

As used herein, the terms “including”, “containing” and “comprising” are used in their open, non-limiting sense.

As used herein, unless otherwise noted, the term “alkyl” refers to a straight- or branched-chain alkyl group having from 1 to 12 carbon atoms in the chain. Examples of alkyl groups include methyl (Me, which also can be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₆alkyl as used here refers to a straight- or branched-chain alkyl group having from 1 to 6 carbon atoms in the chain. The term C₁₋₄alkyl as used here refers to a straight- or branched-chain alkyl group having from 1 to 4 carbon atoms in the chain. The term C₁₋₃alkyl as used here refers to a straight- or branched-chain alkyl group having from 1 to 3 carbon atoms in the chain. The term C₁₋₂alkyl as used here refers to an alkyl group having from 1 to 2 carbon atoms in the chain.

As used herein, unless otherwise noted, the term “C_{X-Y}cycloalkyl”, wherein X and Y are integers, shall mean any stable X- to Y-membered monocyclic, bicyclic, polycyclic, bridged, or spiro-cyclic saturated ring system, preferably a monocyclic, bicyclic, bridged or spiro-cyclic saturated ring system. For example, “C₃₋₇cycloalkyl” refers to a cycloalkyl having from 3 to 7 carbon atoms in the ring(s), “C₃₋₆cycloalkyl” refers to a cycloalkyl having from 3 to 6 carbon atoms in the ring, “C₃₋₄cycloalkyl” refers to a cycloalkyl having from 3 to 4 carbon atoms in the ring. The term “cycloalkyl” also encompasses cycloalkyl group, as defined above, which has been fused to a benzene ring. Illustrative examples of cycloalkyl groups include the following entities, in the form of properly bonded moieties:

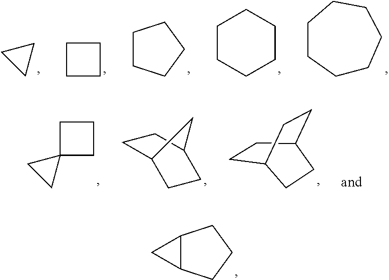

and the like.

As used herein, unless otherwise noted, “cyano” refers to —CN group.

The term “heteroaryl” as used herein, refers to an aromatic monocyclic or multicyclic ring system comprising 5 to 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is multicyclic and has 6 or 14 ring atoms and at least one nitrogen ring atom. A heteroaryl group is joined via a ring carbon atom and any nitrogen atom of a heteroaryl can be optionally oxidized to the corresponding N-oxide.

The term “heteroaryl” also encompasses a heteroaryl group, as defined above, which has been fused to a benzene ring. The term “heteroaryl” also encompasses a heteroaryl group, as defined above, which has been fused to a benzene or another heteroaryl ring.

The term “5-membered heteroaryl” as used herein, refers to a heteroaryl group, as defined above, which has 5 ring atoms. Non-limiting examples of illustrative 5-membered heteroaryls include:

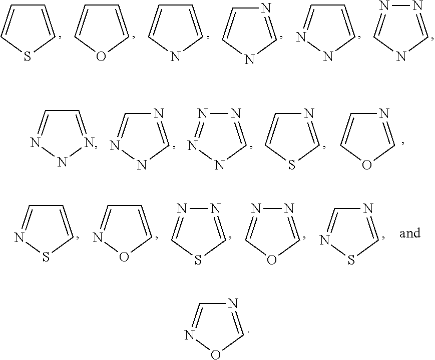

The term “6-membered heteroaryl” as used herein, refers to a heteroaryl group, as defined above, which has 6 ring atoms. Non-limiting examples of illustrative 6-membered heteroaryls include:

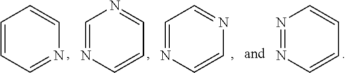

The term “5,6-fused bicyclic heteroaryl or 6,6-fused bicyclic heteroaryl” as used herein, refers to a heteroaryl group, as defined above, which has 9-10 ring atoms. Non-limiting examples of illustrative 5,6-fused bicyclic heteroaryl or 6,6-fused bicyclic heteroaryl include:

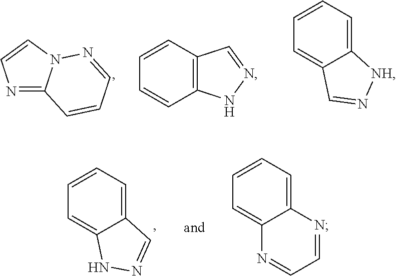

and the like.

Unless otherwise noted, any heteroaryl (regardless of the number of ring atoms, the number and identity of ring heteroatoms, etc.) may be bound through any ring atom which results in a stable structure.

As used herein, unless otherwise noted, the term “heterocycloalkyl” shall denote any monocyclic, bicyclic, spiro-cyclic, bridged or fused saturated or partially unsaturated ring structure containing at least one heteroatom selected from the group consisting of O, N and S, optionally containing one to three additional heteroatoms independently selected from the group consisting of O, N and S. The term “heterocycloalkyl” also encompasses heterocycloalkyl group, as defined above, which has been fused to a benzene or heteroaryl ring. Unless otherwise noted, any heterocycloalkyl (regardless of the number of ring atoms, the number and identity of ring heteroatoms, etc.) may be bound through any ring atom which results in a stable structure.

Illustrative examples of monocyclic heterocycloalkyl include but are not limited to:

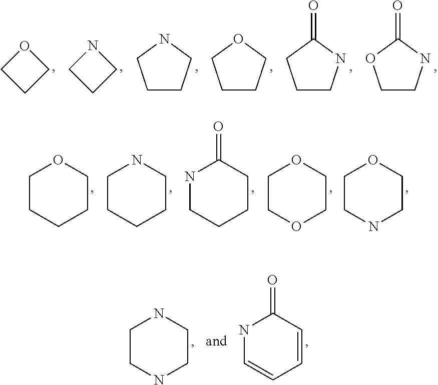

and the like.

Illustrative examples of bridged heterocycloalkyl and spiro-cyclic heterocycloalkyl include, but are not limited to:

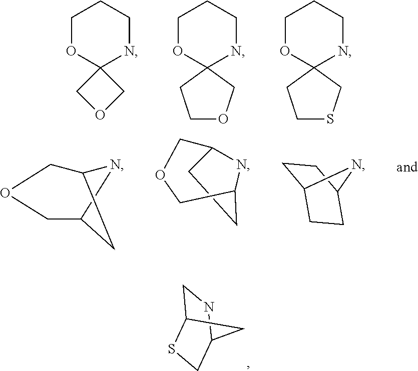

and the like.

As used herein, “halogen” or “halo” means chloro, bromo, fluoro and iodo, preferably bromo, fluoro or chloro, more preferably fluoro or chloro.

As used herein, unless otherwise noted, “hydroxy” refers to —OH group.

“Haloalkyl” refers to a straight- or branched-chain alkyl group having from 1 to 12 carbon atoms in the chain optionally substituting hydrogens with halogens. The term “C₁₋₆haloalkyl” as used here refers to a straight- or branched-chain alkyl group having from 1 to 6 carbon atoms in the chain, substituting one or more hydrogens with halogens. The term “C₁₋₃haloalkyl” as used here refers to a straight- or branched-chain alkyl group having from 1 to 3 carbon atoms in the chain, substituting one or more hydrogens with halogens. Examples of haloalkyl groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (—CF(CF₃)₂), and groups that in light of the ordinary skill in the art and the teachings provided herein would be considered equivalent to any one of the foregoing examples.

“Phenyl” refers to the following moiety:

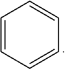

Lines drawn into ring systems indicate that the bond may be attached to any of the suitable and available ring atoms. The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

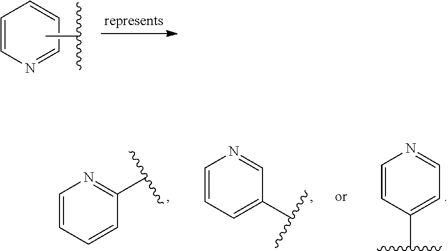

Those skilled in the art will recognize that if more than one such substituent is present for a given ring, the bonding of each substituent is independent of all of the others. The groups listed or illustrated above are not exhaustive.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this disclosure may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R) refers to a compound that is a pure single isomer at that stereocenter with an absolute configuration of either (R) or (S), and a compound designated as (S) refers to a compound that is a pure single isomer at that stereocenter with an absolute configuration of either (R) or (S). For example, (2S,4S)-1-(2-(Difluoromethyl)-9-fluorobenzofuro[3,2-d]pyrimidin-4-yl)-4-((5-((1-methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid:

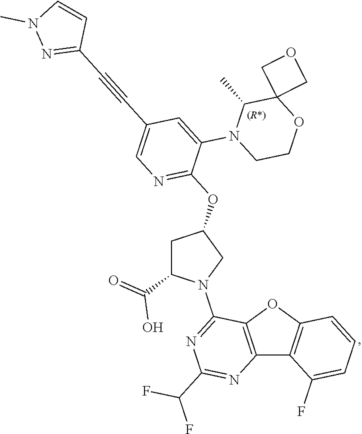

refers to a compound that is either:

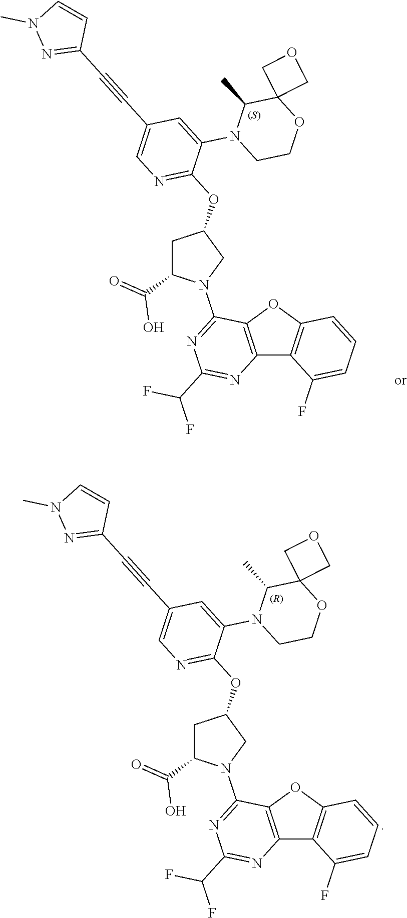

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH (aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the present disclosure in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O, ³¹p, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this present disclosure can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for such variable appearing elsewhere. In other words, where a variable appears more than once, the choice of the species from a specified list is independent of the choice of the species for the same variable elsewhere in the formula, unless stated otherwise.

The term “zwitterion” refers to a molecule having a net formal charge of zero, but negative and positive formal charges on individual atoms within its structure. The charged atoms are joined by one or more covalent bonds. In some embodiments, described herein are compounds of Formula (I) or Formula (II) that can exist in zwitterionic form.

The term Cₙ₋ₘ alkyl refers to an aliphatic chain, whether straight or branched, with a total number N of carbon members in the chain that satisfies n≤N≤m, with m>n.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

The nomenclature “Cᵢ-Cⱼ” or “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃).

A “pharmaceutically acceptable salt” is intended to mean a salt of an acid or base of a compound represented by Formula (I) or a compound of Formula (II) (including compounds of Formulas (IIA), (IIB), (IIC), (IIC′), and (IID)) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

A compound of Formula (I) or a compound of Formula (II) (including compounds of Formulas (IIA), (IIB), (IIC), (IIC′), and (IID)) may possess a sufficiently acidic group, a sufficiently basic group, or both types of functional groups, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Compounds of Formula (I) or compounds of Formula (II) (including compounds of Formulas (IIA), (IIB), (IIC), (IIC′), and (IID)) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) or compounds of Formula (II) (including compounds of Formulas (IIA), (IIB), (IIC), (IIC′), and (IID)) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

The compounds of the present disclosure, including their pharmaceutically acceptable salts, whether alone or in combination, (collectively, “active agent” or “active agents”) of the present disclosure are useful as cGAS inhibitors in the methods of the present disclosure.

Such methods for inhibiting cGAS comprise the use of a therapeutically effective amount of at least one chemical entity of the present disclosure.

In some embodiments, the cGAS inhibitor and is used in a subject diagnosed with or suffering from a disease, disorder, or condition associated with cGAS modulation, such as those described herein. Symptoms or disease states are intended to be included within the scope of “disease, disorders or conditions.”

Accordingly, the present disclosure relates to methods of using the active agents described herein to treat subjects diagnosed with or suffering from a disease, disorder, or condition associated with the cGAS modulation. The term “treat” or “treating” as used herein is intended to refer to administration of an active agent or composition of the present disclosure to a subject for the purpose of effecting a therapeutic or prophylactic benefit through modulation of cGAS modulation. Treating includes reversing, ameliorating, alleviating, inhibiting the progress of, lessening the severity of, or preventing a disease, disorder, or condition, or one or more symptoms of such disease, disorder or condition associated with cGAS modulation. The term “subject” refers to a mammalian patient in need of such treatment, such as a human.

The term “composition” refers to a product that includes the specified ingredients in therapeutically effective amounts, as well as any product that results, directly, or indirectly, from combinations of the specified ingredients in the specified amounts.

The term “cGAS inhibitor” is intended to encompass a compound that interacts with the cGAS pathway to substantially reduce or eliminate its activity. The disclosure is directed to methods for treating, ameliorating and/or preventing diseases, conditions, or disorders associated with autoimmune disorders including Aicardi-Goutieres Syndrome (AGS), Systemic Lupus Erythematosus (SLE), Lupus Nephritis, Scleroderma, Sjogren's Syndrome, Inflammatory Myopathies, Hidradenitis Supperativa (HS), Parkinson's Disease, Rheumatoid Arthritis, Ulcerative Colitis and Crohn's Disease by the administration of therapeutically effective amounts of cGAS modulators to subjects in need thereof.

The term “modulators” include both inhibitors and activators, where “inhibitors” refer to compounds that decrease, prevent, inactivate, desensitize, and/or down-regulate the cGAS expression or activity, and “activators” are compounds that increase, activate, facilitate, sensitize, or up-regulate cGAS activity.

As used herein, unless otherwise noted, the term “affect” or “affected” (when referring to a disease, condition or disorder that is affected by inhibition of cGAS) includes a reduction in the frequency and/or severity of one or more symptoms or manifestations of said disease, condition or disorder; and/or include the prevention of the development of one or more symptoms or manifestations of said disease, condition or disorder or the development of the disease, condition or disorder.

In an attempt to help the reader of the application, the description has been separated in various paragraphs or sections or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

### Compounds of the Present Disclosure

Embodiments of the present disclosure are compounds of Formula (I),

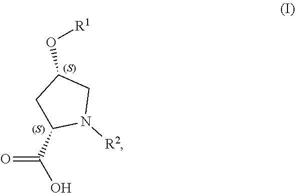

or a pharmaceutically acceptable salt and/or stereoisomer thereof,
wherein:
R¹ is selected from:

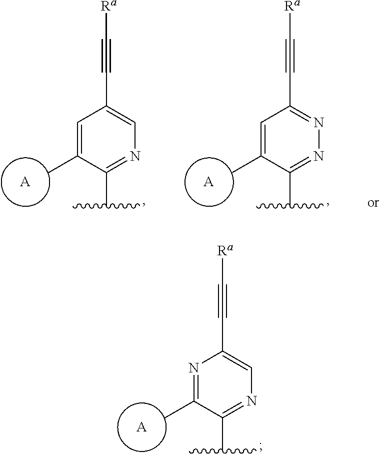

wherein \textcircled{A} is a nitrogen linked monocyclic heterocycloalkyl, spiro-heterocycloalkyl, or bridged heterocycloalkyl selected from the group consisting of:

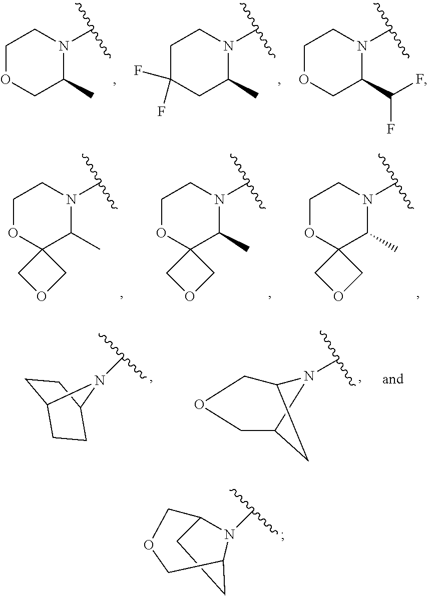

wherein R^{a} is selected from the group consisting of:
   (i) —C₁₋₆alkyl optionally substituted with one, two, or three members each independently selected from the group consisting of: —F, —OH, —OC₁₋₆alkyl, —NH(SO₂CH₃), —OCH₂CH₂OCH₃, and —N(C₁₋₄alkyl)₂; or
   (ii) —C₃₋₆cycloalkyl or -L¹-C₃₋₆cycloalkyl, wherein the cycloalkyl is monocyclic, spirocyclic, bridged or fused, optionally substituted with one, two, or three members each independently selected from the group consisting of: —F, —C₁₋₆alkyl, —C₁₋₆haloalkyl, —OC₁₋₆alkyl, and 1H-pyrazole; wherein -L¹- is —C(H)(OCH₃)—or —C₁₋₃alkyl; or
   (iii) phenyl or -L²-phenyl, wherein the phenyl is optionally substituted with one, two, or three members each independently selected from the group consisting of: —F, —C₁₋₆alkyl, —C₁₋₆haloalkyl, —OC₁₋₆alkyl, —N(C₁₋₄alkyl)₂, and 4-methyl-1H-imidazole; wherein -L²- is —CH₂O—; or
   (iv) heterocycloalkyl or -L³-heterocycloalkyl; wherein the heterocycloalkyl is selected from the group consisting of:

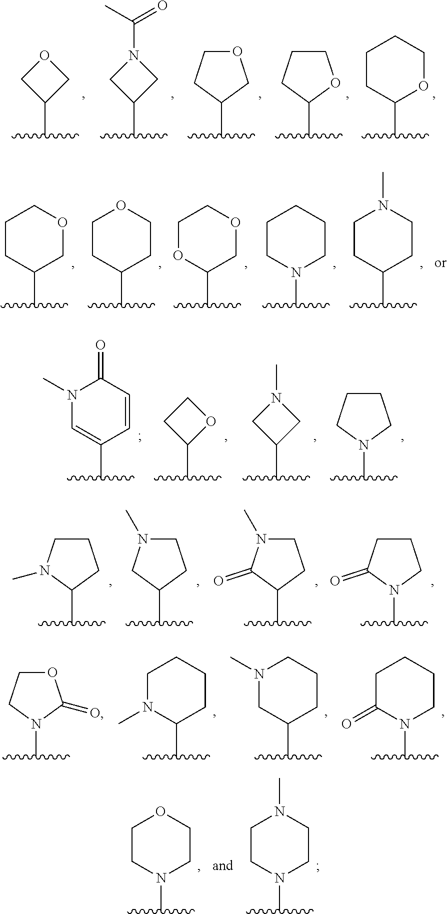

wherein each heterocycloalkyl is optionally substituted with one, two, or three members each independently selected from: F, —OH, —C₁₋₆alkyl, —C₁₋₆haloalkyl, and —N(C₁₋₄alkyl)₂; wherein -L₃- is —C₁₋₃alkyl-, or —CH₂O—; or
(v) 5-membered heteroaryl or -L³-(5-membered heteroaryl), wherein the 5-membered heteroaryl is selected from the group consisting of:

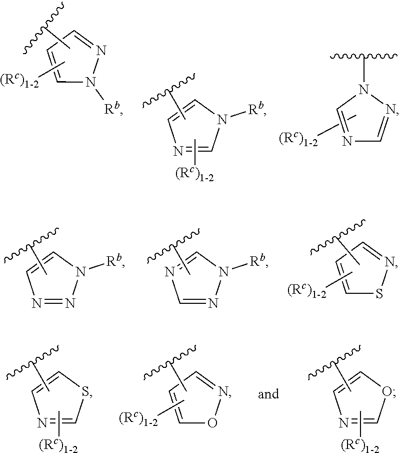

wherein R^{b} is H, or —C₁₋₆alkyl; each R^{c} is independently selected from the group consisting of: H, —C₁₋₆alkyl, —C₁₋₆haloalkyl, —OC₁₋₆alkyl, —CH₂OCH₃, and cyclopropyl; wherein -L³- is —C₁₋₃alkyl-, or —CH₂O—; or
(vi) 6-membered heteroaryl or -L⁴-(6-membered heteroaryl); wherein the 6-membered heteroaryl is selected from the group consisting of:

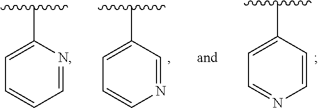

wherein each 6-membered heteroaryl is optionally substituted with one, two, or three members each independently selected from the group consisting of: —F, —C₁₋₆alkyl, —C₁₋₆haloalkyl, —OC₁₋₆haloalkyl, and —OC₁₋₆alkyl; wherein -L⁴- is —C₁₋₃alkyl-, or —CH₂O—; or
(vii) 5,6-fused bicyclic heteroaryl or 6,6-fused bicyclic heteroaryl selected from the group consisting of:

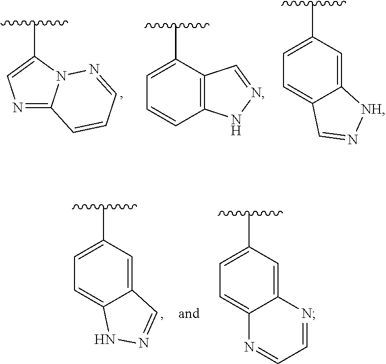

and
R² is

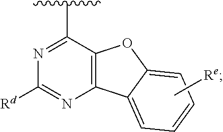

wherein
R^{d} is selected from the group consisting of: —C₁₋₆alkyl; —C₁₋₆haloalkyl; —OC₁₋₆alkyl; —C₃₋₆cycloalkyl; —N(C₁₋₄alkyl)₂;
-heterocycloalkyl optionally substituted with one or two substituents each independently selected from halo, and —Cᵢ-alkyl; and

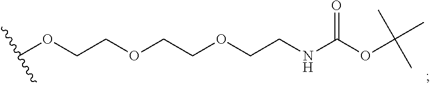 [SMILES: *OCCOCCOCCNC(=O)OC(C)(C)C]

and
R^{e} is selected from the group consisting of: H, F, —C₁₋₆alkyl, and —C₁₋₆haloalkyl.

In some embodiments, disclosed herein is a compound of Formula (II), or a pharmaceutically acceptable salt thereof,

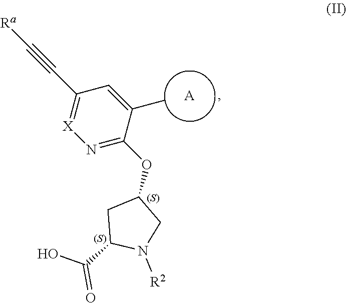

wherein:
\textcircled{A} is

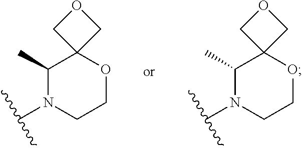 [SMILES: *N1CCOC2(COC2)[C@@H]1C ; *N1CCOC2(COC2)[C@H]1C]

X is CH or N;
R^{a} is selected from the group consisting of:
   (i) —C₁₋₆alkyl substituted with one or two members each independently selected from the group consisting of: —OH, and —OC₁₋₆alkyl; or
   (ii) —C₃₋₆cycloalkyl or -L¹-C₃₋₆cycloalkyl, wherein the cycloalkyl is monocyclic or fused bicyclic, and unsubstituted or substituted with one or two members each independently selected from the group consisting of: —F, —OH, —OC₁₋₆alkyl, and 1H-pyrazole; wherein -L¹- is —C₁₋₃alkyl; or
   (iii) phenyl or -L²-phenyl, wherein the phenyl is unsubstituted or substituted with —C₁₋₆alkyl, or —OC₁₋₆alkyl; wherein -L²- is —CH₂O—; or
   (iv) heterocycloalkyl or -L³-heterocycloalkyl; wherein the heterocycloalkyl is selected from the group consisting of:

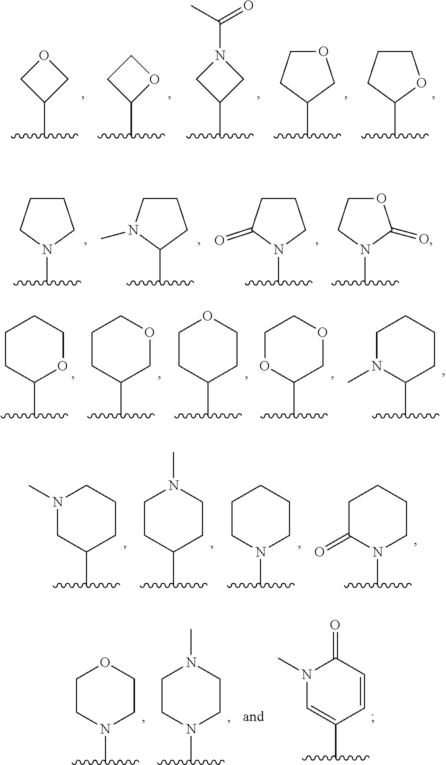

wherein each heterocycloalkyl is unsubstituted or substituted with one or two members each independently selected from: —F, —OH, —CH₂OH, and —C₁₋₆alkyl; wherein —L₃- is —C₁₋₃alkyl-; or
(v) 5-membered heteroaryl or -L³-(5-membered heteroaryl), wherein the 5-membered heteroaryl is selected from the group consisting of:

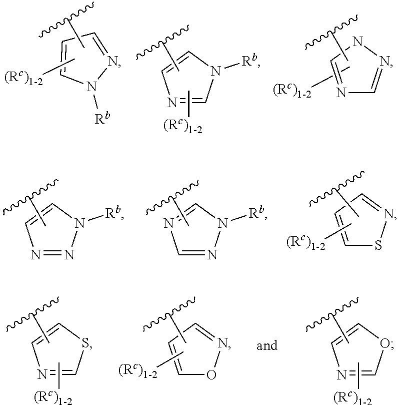

wherein R^{b} is —C₁₋₆alkyl or cyclopropyl; each R^{c} is independently selected from the group consisting of: H, —C₁₋₆alkyl, —OC₁₋₆alkyl, and cyclopropyl; wherein -L³- is —C₁₋₃alkyl; or
(vi) 6-membered heteroaryl; wherein the 6-membered heteroaryl is selected from the group consisting of:

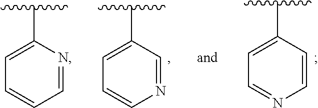

wherein each 6-membered heteroaryl is unsubstituted or substituted with one or two members each independently selected from the group consisting of: —F, —C₁₋₃alkyl, —C₁₋₃haloalkyl, and —OC₁₋₆alkyl; or
(vii)

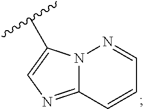 [SMILES: *c1cnc2cccnn12]

and
R² is

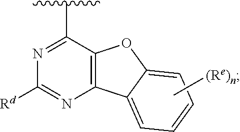

wherein
R^{d} is selected from the group consisting of: —C₁₋₆alkyl; —C₁₋₆haloalkyl; —OC₁₋₆alkyl; -cyclopropyl; heterocycloalkyl, wherein the hetercycloalkyl- is selected from the group consisting of:

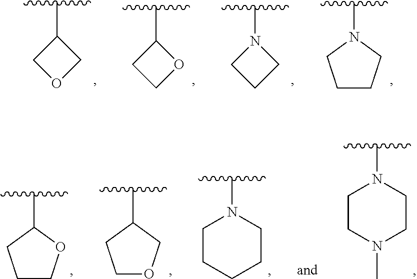

wherein each heterocycloalkyl is unsubstituted or substituted with one or two substituents each independently selected from halo, and —C₁₋₆alkyl; and

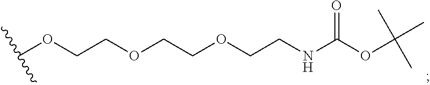 [SMILES: *OCCOCCOCCNC(=O)OC(C)(C)C]

R^{e} is selected from the group consisting of: —F, —C₁₋₃alkyl, and —C₁₋₃alkyl; and
n is 0, 1, or 2.

An additional embodiment of the present disclosure is a compound of Formula (II) wherein X is CH and \textcircled{A} is

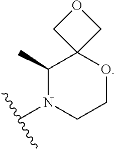 [SMILES: *N1CCOC2(COC2)[C@@H]1C]

An additional embodiment of the present disclosure is a compound of Formula (II) wherein X is N.

An additional embodiment of the present disclosure is a compound of Formula (II) wherein R^{a} is

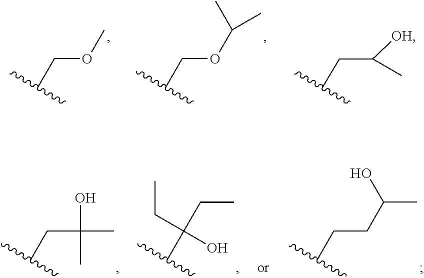

An additional embodiment of the present disclosure is a compound of Formula (II) wherein R^{a} is

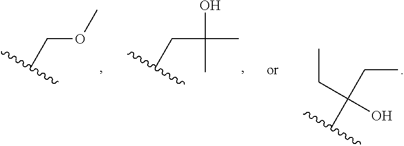

An additional embodiment of the present disclosure is a compound of Formula (II) wherein R^{a} is

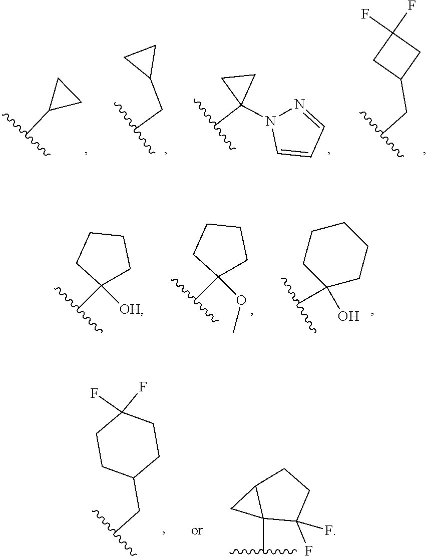

An additional embodiment of the present disclosure is a compound of Formula (II) wherein R^{a}

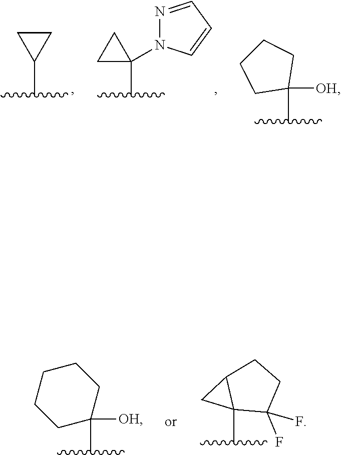

An additional embodiment of the present disclosure is a compound of Formula (II) wherein R^{a}

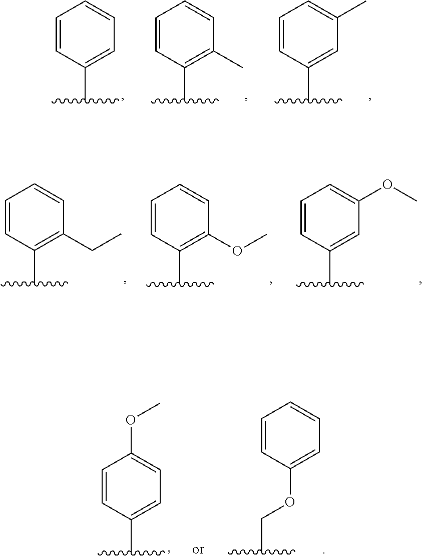

An additional embodiment of the present disclosure is a compound of Formula (II) wherein R^{a} is

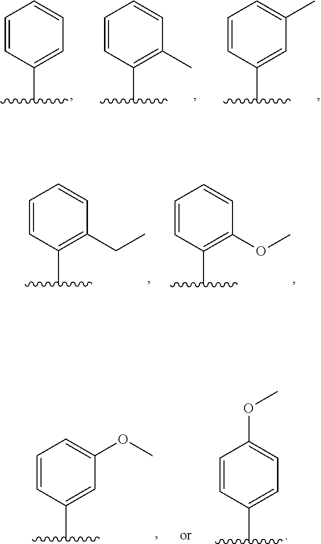

An additional embodiment of the present disclosure is a compound of Formula (II) wherein R^{a} is

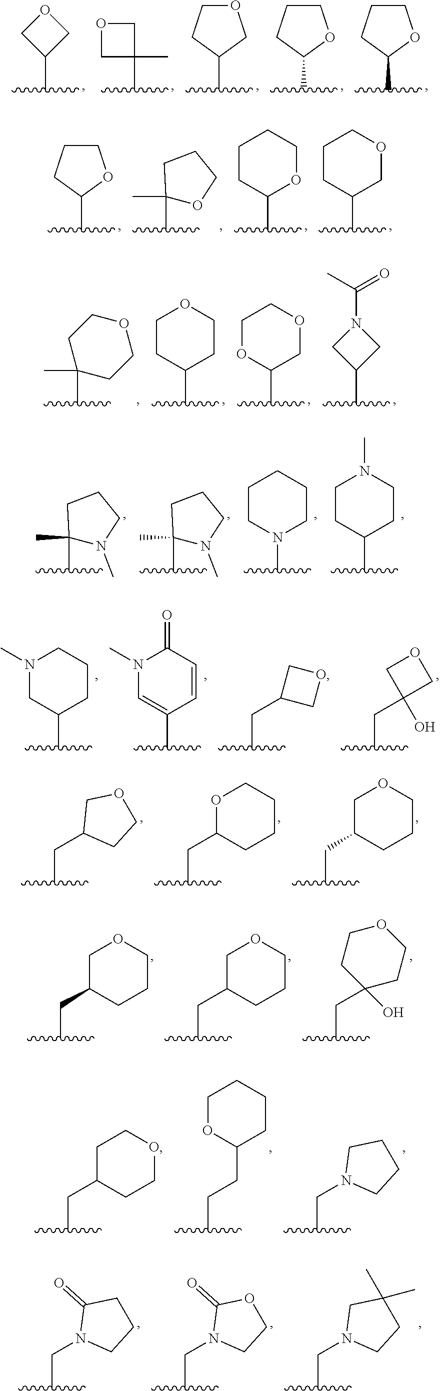

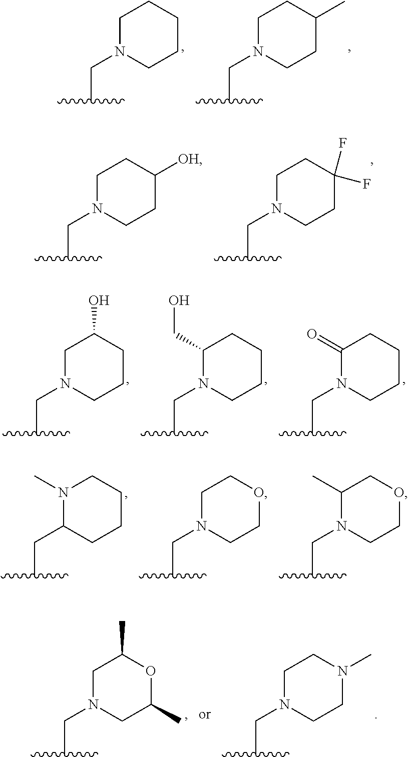

An additional embodiment of the present disclosure is a compound of Formula (II) wherein R^{a} is

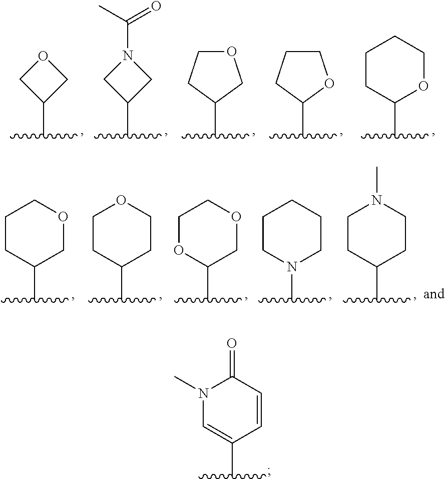

An additional embodiment of the present disclosure is a compound of Formula (II) wherein R^{a} is

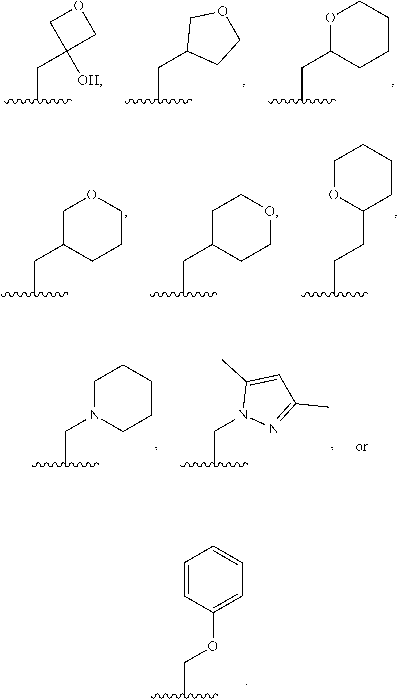

An additional embodiment of the present disclosure is a compound of Formula (II) wherein R^{a}
is

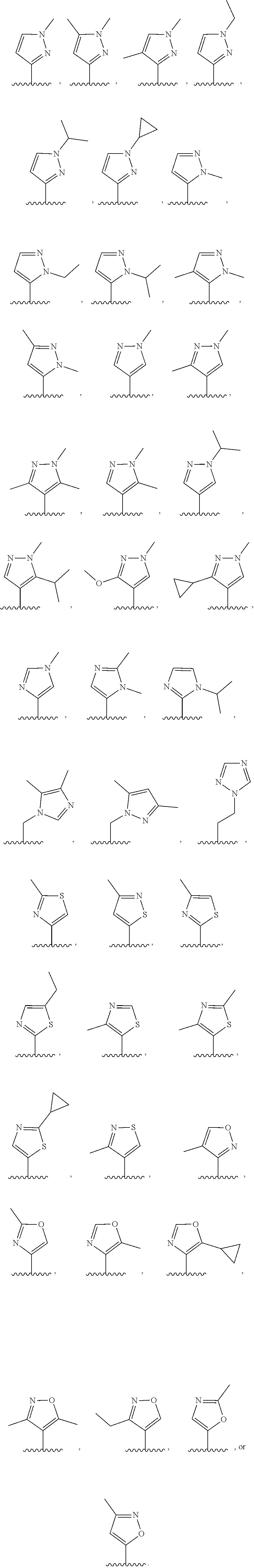

An additional embodiment of the present disclosure is a compound of Formula (II) wherein R^{a} is

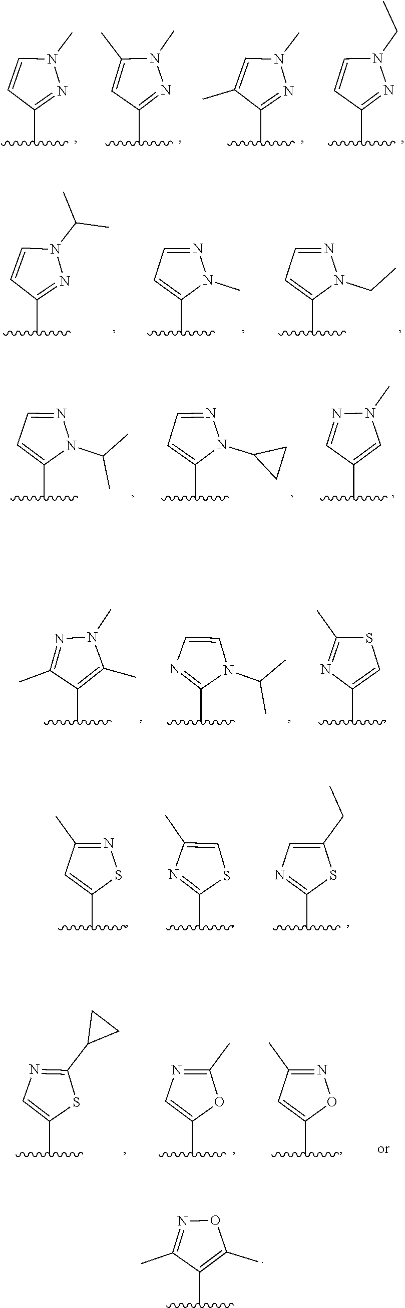

In some embodiments of the present disclosure is a compound of Formula

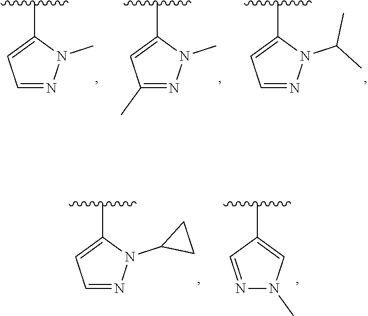

(II) wherein R^{a} is:

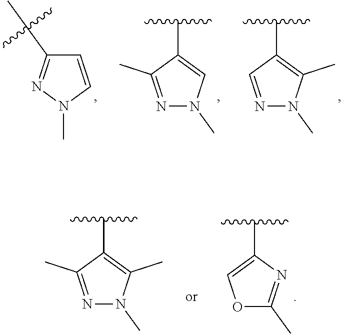

In some embodiments of the present disclosure is a compound of Formula (II) wherein R^{a} is:

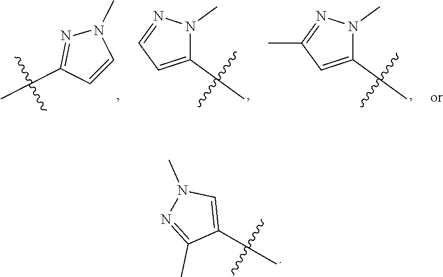

In some embodiments of the present disclosure is a compound of Formula (II) wherein R^{a} is:

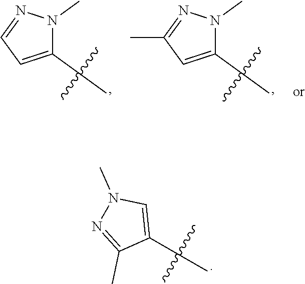

In some embodiments of the present disclosure is a compound of Formula (II) wherein R^{a}
is:

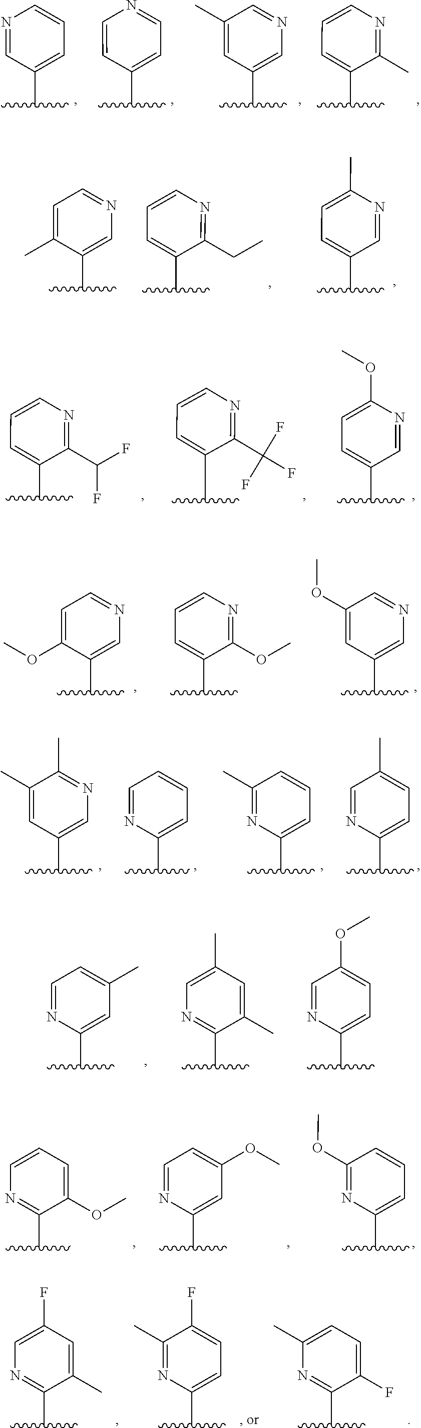

An additional embodiment of the present disclosure is a compound of Formula (II) wherein R^{a} is:

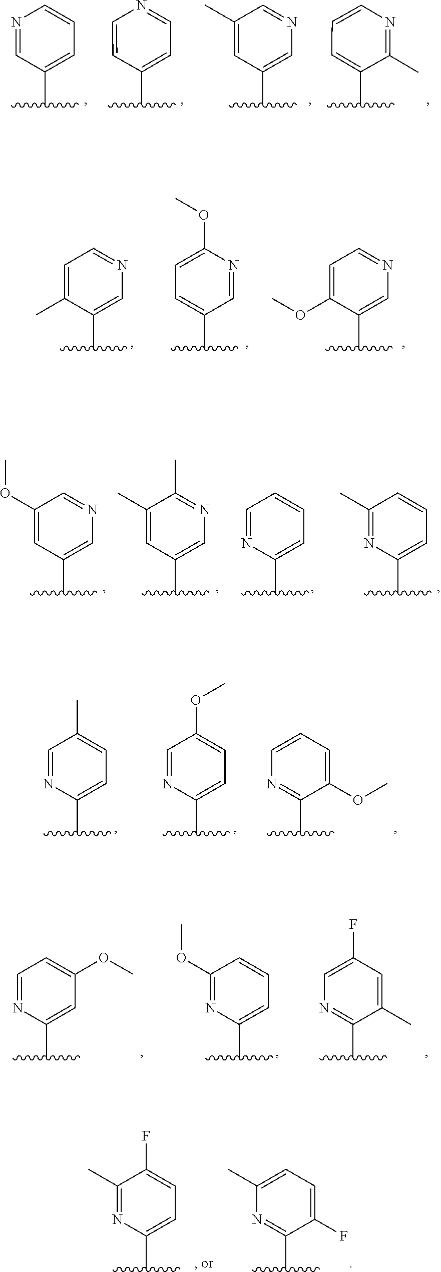

In some embodiments of the present disclosure is a compound of Formula (II) wherein R^{a} is:

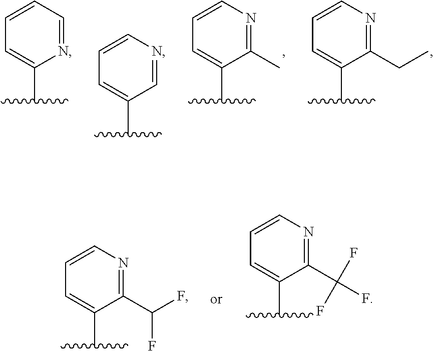

In some embodiments of the present disclosure is a compound of Formula (II) wherein R^{a} is

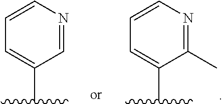

In some embodiments of the present disclosure is a compound of Formula (II) wherein R^{a} is

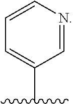 [SMILES: *c1cccnc1]

In some embodiments of the present disclosure is a compound of Formula (II) wherein R^{a} is

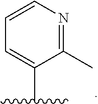 [SMILES: *c1cccnc1C]

An additional embodiment of the present disclosure is a compound of Formula (II) wherein R^{a} is

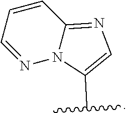 [SMILES: *c1cnc2cccnn12]

An additional embodiment of the present disclosure is a compound of Formula (II) wherein R² is

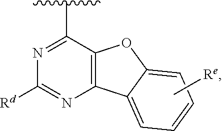

and R^{e} is H, F, —CH₃, or —CF₃.

An additional embodiment of the resent disclosure is a compound of Formula (II) wherein R² is

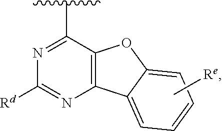

and R^{d} is —CH(CH₃)₂, —C(CH₃)₃, —OCH₃, —OCH₂CH₃, or —CF₂H.

An additional embodiment of the present disclosure is a compound of Formula (II) wherein R² is

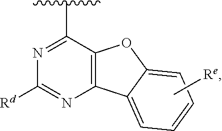

and R^{d} is —CH₃, —CH₂CH₃, —CH(CH₃)₂, or —CF₂H.

An additional embodiment of the present disclosure is a compound of Formula (II) wherein R² is

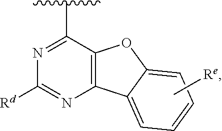

and R^{d} is —CH₃, —CH₂CH₃, or —CH(CH₃)₂.

An additional embodiment of the present disclosure is a compound of Formula (II) wherein R² is

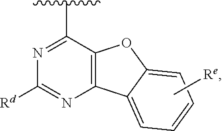

R^{e} is H and R^{d} is —CH₃, or —CH₂CH₃.

An additional embodiment of the present disclosure is a compound of Formula (II) wherein R² is

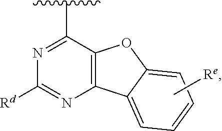

R^{e} is H and R^{d} is CH₂CH₃.

An additional embodiment of the present disclosure is a compound of Formula (II) wherein R² is

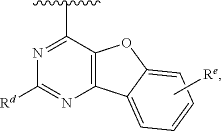

and R^{d} is cyclopropyl or

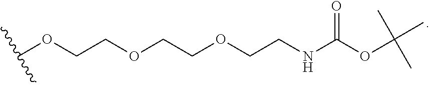 [SMILES: *OCCOCCOCCNC(=O)OC(C)(C)C]

In some embodiments, disclosed herein is a compound of Formula (IIA), or a pharmaceutically acceptable salt thereof, wherein the compound is:

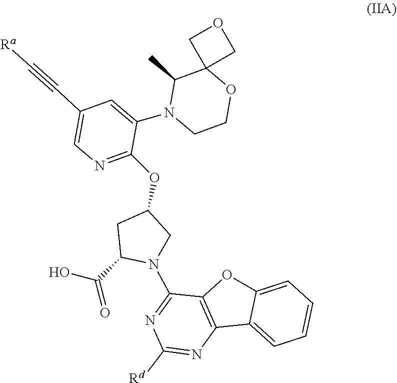

wherein
R^{a} is
   (i) phenyl, wherein the phenyl is unsubstituted or substituted with one member selected from the group consisting of: —C₁₋₆alkyl or —OCH₃; or
   (ii) 5-membered heteroaryl; wherein the 5-membered heteroaryl is selected from the

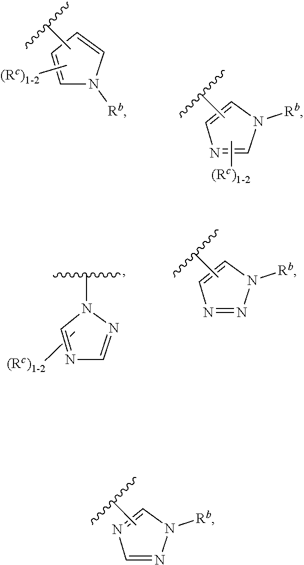

group consisting of:

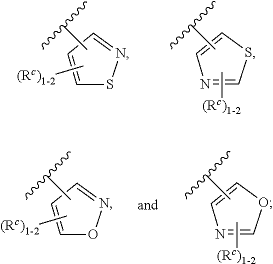

wherein R^{b} is —C₁₋₃alkyl or cyclopropyl; each R^{c} is independently selected from the group consisting of: H, —C₁₋₃alkyl, —OCH₃, and cyclopropyl; or
(iii) 6-membered heteroaryl; wherein the 6-membered heteroaryl is selected from the group consisting of:

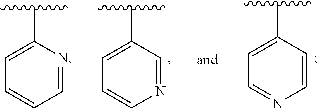

wherein each 6-membered heteroaryl is unsubstituted or substituted with one or two members each independently selected from the group consisting of: —F, —C₁₋₃alkyl, —CF₂H, —CF₃, and —OCH₃; and
R^{d} is
   (i) —CH₃, —CH₂CH₃, —CH(CH₃)₂, —C(CHs)₃, —CHF₂, —CF₃, —OCH₃, —OCH₂CH₃, —CH(CH₃)(CF₃) or

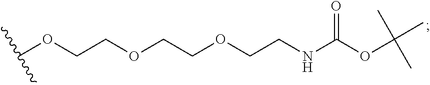 [SMILES: *OCCOCCOCCNC(=O)OC(C)(C)C]

or
cyclopropyl; or

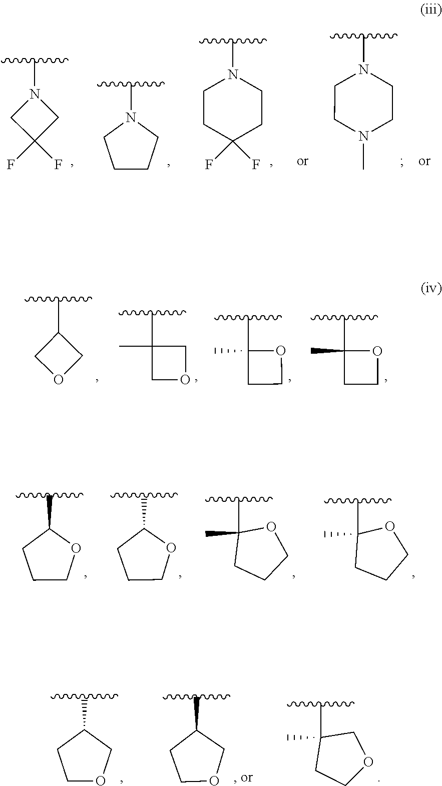

In some embodiments, disclosed herein is a compound of Formula (IIA), or a pharmaceutically acceptable salt thereof, wherein:
R^{a} is

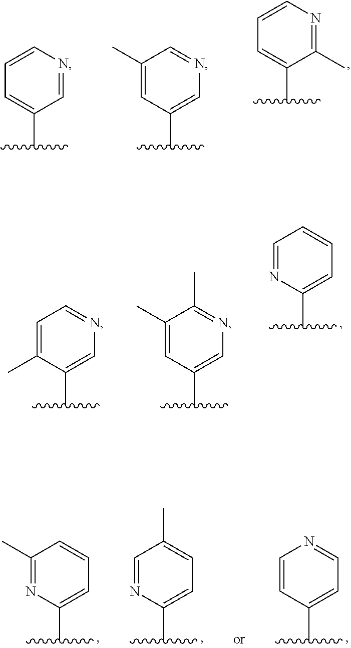

and
R^{d} is —CH₃, —CH₂CH₃, —CH(CH₃)₂, —C(CH₃)₃, or —CHF₂.

In some embodiments, disclosed herein is a compound of Formula (IIA), or a pharmaceutically acceptable salt thereof, wherein:
R^{a} is

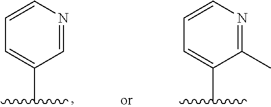

and R^{d} is —CH₃, —CH₂CH₃, —CH(CH₃)₂, or —CHF₂.

In some embodiments, disclosed herein is a compound of Formula (IIA), or a pharmaceutically acceptable salt thereof, wherein:
R^{a} is

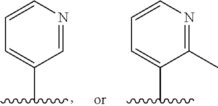

and R^{d} is —CH₂CH₃.

In some embodiments, disclosed herein is a compound of Formula (IIA), or a pharmaceutically acceptable salt thereof, wherein:
R^{a} is

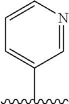 [SMILES: *c1cccnc1]

and R^{d} is —CH₂CH₃.

In some embodiments, disclosed herein is a compound of Formula (IIA), or a pharmaceutically acceptable salt thereof, wherein:

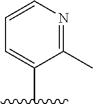 [SMILES: *c1cccnc1C]

R^{a} is

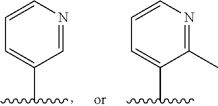

and R^{d} is —CH₂CH₃.

In some embodiments, disclosed herein is a compound of Formula (IIA), or a pharmaceutically acceptable salt thereof, wherein:
R^{a} is

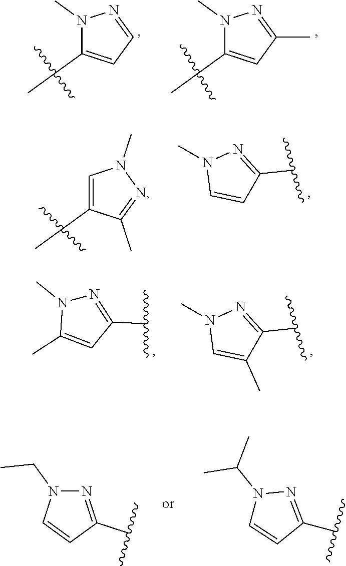

R^{d} is —CH₃, —CH₂CH₃, —CH(CH₃)₂, —C(CH₃)₃, or —CHF₂.

In some embodiments, disclosed herein is a compound of Formula (IIA), or a pharmaceutically acceptable salt thereof, wherein:
R^{a} is

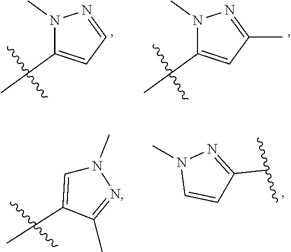

and
R^{d} is —CH₃, —CH₂CH₃, —CH(CH₃)₂, or —C(CH₃)₃.

In some embodiments, disclosed herein is a compound of Formula (IIA), or a pharmaceutically acceptable salt thereof, wherein:
R^{a} is

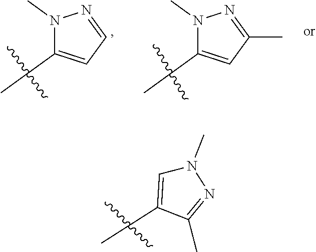

and R^{d} is —CHs or —CH₂CH₃.

In some embodiments, disclosed herein is a compound of Formula (IIA), or a pharmaceutically acceptable salt thereof, wherein:
R^{a} is

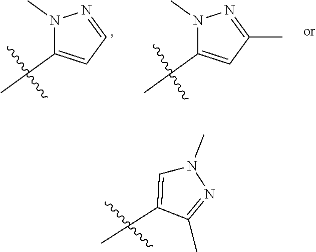

and R^{d} is —CH₂CH₃.

In some embodiments, disclosed herein is a compound of Formula (IIA), or a pharmaceutically acceptable salt thereof, wherein:
R^{a} is

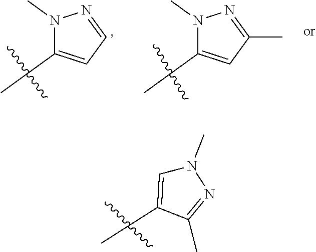

and R^{d} is —CH₃.

In some embodiments, disclosed herein is a compound of Formula (IIB), or a pharmaceutically acceptable salt thereof,

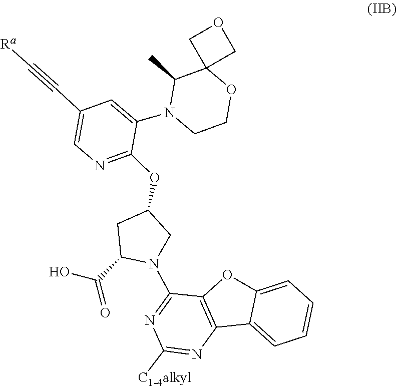

wherein
R^{a} is
   (iii) 5-membered heteroaryl; wherein the 5-membered heteroaryl is selected from the group consisting of:

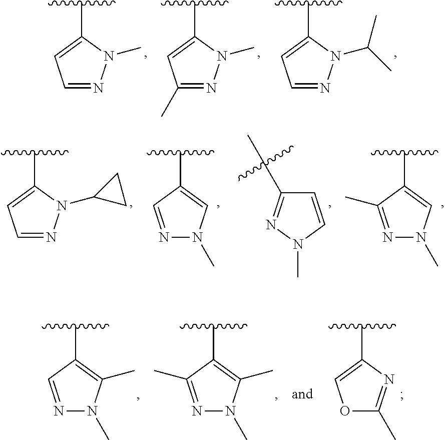

or
(iv) 6-membered heteroaryl; wherein the 6-membered heteroaryl is selected from the group consisting of:

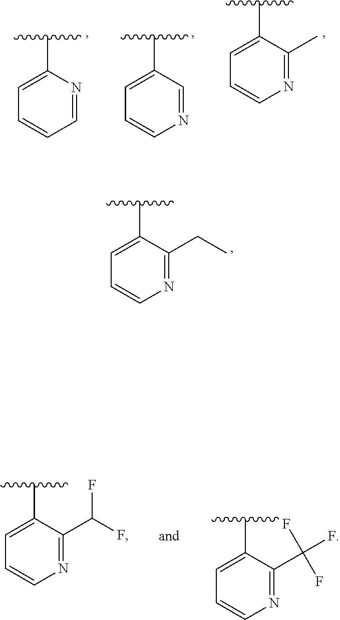

In some embodiments, disclosed herein is a compound of Formula (IIC), or a pharmaceutically acceptable salt thereof,

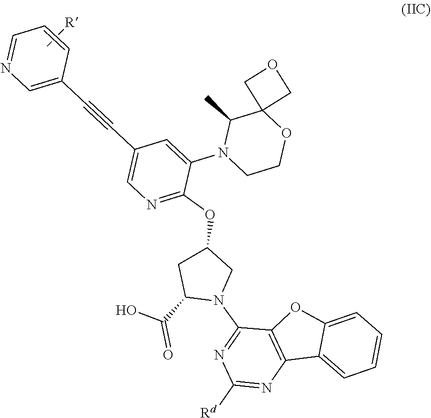

wherein R′ is —CH₃ and R^{d} is —CH₃ or —CH₂CH₃.

In some embodiments, compounds of Formula (IIC) are compounds of Formula (IIC′):

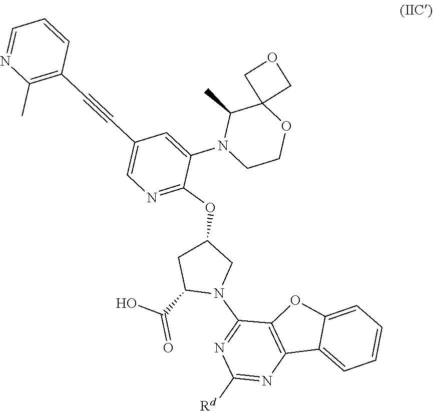

wherein R^{d} is —CH₃ or —CH₂CH₃. In some embodiments, disclosed herein are compounds of Formula (IIC′) wherein R^{d} is —CH₂CH₃. In some embodiments, disclosed herein are compounds of Formula (IIC′) wherein R^{d} is —CH₃.

In some embodiments, disclosed herein is a compound of Formula (IID), or a pharmaceutically acceptable salt thereof,

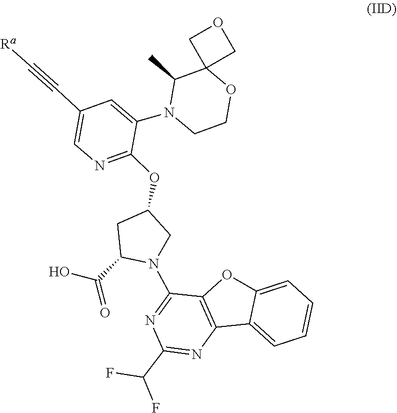

wherein
R^{a} is:
   (i) —C₃₋₆cycloalkyl or -L¹-C₃₋₆cycloalkyl, wherein the cycloalkyl selected from the group consisting of:

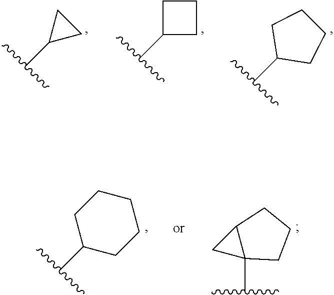

wherein each cycloalkyl is unsubstituted or substituted with one, or two members each independently selected from the group consisting of: —F, —OH, —OCH₃, and 1H-pyrazole; and wherein L¹ is CH₂; or
(ii) heterocycloalkyl or -L³-heterocycloalkyl; wherein the heterocycloalkyl is selected from the group consisting of:

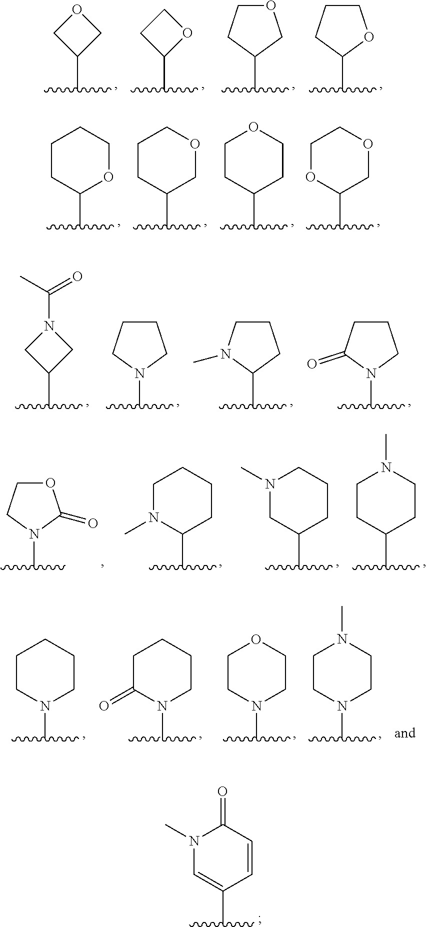

wherein each heterocycloalkyl is unsubstituted or substituted with one or two members each independently selected from: —F, —OH, —CH₂OH, and —C₁₋₃alkyl; and wherein -L³- is —CH₂—, or —CH₂CH₂—.

In certain embodiments, the present disclosure is directed to one or more compounds of Formula (I) or Formula (II) independently selected from the group consisting of the compounds of Table 1.

TABLE 1
Representative Compounds for Formula (I) or Formula (II).

| Ex # | Compound Name |
|---|---|
| 1 | (2S,4S)-1-(2-(Difluoromethyl)benzofuro[3,2-d]pyrimidin-4-yl)-4-((5-(3- |
| | methoxyprop-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 2 | (2S,4S)-1-(2-(Difluoromethyl)benzofuro[3,2-d]pyrimidin-4-yl)-4-((5-(4-hydroxy-4- |
| | methylpent-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 3 | (2S,4S)-1-(2-(Difluoromethyl)benzofuro[3,2-d]pyrimidin-4-yl)-4-((5-(3-ethyl-3- |
| | hydroxypent-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 4 | (2S,4S)-4-((5-(Cyclopropylethynyl)-3-((S)-9-methyl-2,5-dioxa-8- |
| | azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 5 | (2S,4S)-4-((5-((1-(1H-Pyrazol-1-yl)cyclopropyl)ethynyl)-3-((S)-9-methyl-2,5-dioxa- |
| | 8-azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 6 | (2S,4S)-1-(2-(Difluoromethyl)benzofuro[3,2-d]pyrimidin-4-yl)-4-((5-((1- |
| | hydroxycyclopentyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 7 | (2S,4S)-1-(2-(Difluoromethyl)benzofuro[3,2-d]pyrimidin-4-yl)-4-((5-((1- |
| | hydroxycyclohexyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 8 | (2S,4S)-4-((5-((2,2-Difluorobicyclo[3.1.0]hexan-1-yl)ethynyl)-3-((S)-9-methyl-2,5- |
| | dioxa-8-azaspiro[3.5]nonan-8-yl)pyridin-2-yl)oxy)-1-(2- |
| | (difluoromethyl)benzofuro[3,2-d]pyrimidin-4-yl)pyrrolidine-2-carboxylic acid; |
| 9 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(phenylethynyl)pyridin-2- |
| | yl)oxy)pyrrolidine-2-carboxylic acid; |
| 11 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(o-tolylethynyl)pyridin-2- |
| | yl)oxy)pyrrolidine-2-carboxylic acid; |
| 12 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(m-tolylethynyl)pyridin-2- |
| | yl)oxy)pyrrolidine-2-carboxylic acid; |
| 13 | (2S,4S)-1-(2-(Difluoromethyl)benzofuro[3,2-d]pyrimidin-4-yl)-4-((5-((2- |
| | ethylphenyl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 14 | (2S,4S)-1-(2-(Difluoromethyl)benzofuro[3,2-d]pyrimidin-4-yl)-4-((5-((3- |
| | methoxyphenyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 15 | (2S,4S)-1-(2-(Difluoromethyl)benzofuro[3,2-d]pyrimidin-4-yl)-4-((5-((4- |
| | methoxyphenyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 16 | (2S,4S)-1-(2-(Difluoromethyl)benzofuro[3,2-d]pyrimidin-4-yl)-4-((5-((2- |
| | methoxyphenyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 17 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-phenoxyprop-1-yn-1-yl)pyridin-2- |
| | yl)oxy)pyrrolidine-2-carboxylic acid; |
| 18 | (2S,4S)-4-((5-((1-Acetylazetidin-3-yl)ethynyl)-3-((S)-9-methyl-2,5-dioxa-8- |
| | azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 19 | (2S,4S)-1-(2-(Difluoromethyl)benzofuro[3,2-d]pyrimidin-4-yl)-4-((5-(3-(3- |
| | hydroxyoxetan-3-yl)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 20 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((tetrahydrofuran-3-yl)ethynyl)pyridin-2- |
| | yl)oxy)pyrrolidine-2-carboxylic acid; |
| 21 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((tetrahydrofuran-2-yl)ethynyl)pyridin-2- |
| | yl)oxy)pyrrolidine-2-carboxylic acid; |
| 22 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(tetrahydrofuran-3-yl)prop-1-yn-1- |
| | yl)pyridin-2-yl)oxy)pyrrolidine-2-carboxylic acid; |
| 23 | (2S,4S)-1-(2-(Difluoromethyl)benzofuro[3,2-d]pyrimidin-4-yl)-4-((5-((3- |
| | hydroxytetrahydrofuran-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 24 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((tetrahydro-2H-pyran-4-yl)ethynyl)pyridin- |
| | 2-yl)oxy)pyrrolidine-2-carboxylic acid; |
| 25 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((tetrahydro-2H-pyran-3-yl)ethynyl)pyridin- |
| | 2-yl)oxy)pyrrolidine-2-carboxylic acid; |
| 26 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(tetrahydro-2H-pyran-2-yl)prop-1-yn-1- |
| | yl)pyridin-2-yl)oxy)pyrrolidine-2-carboxylic acid; |
| 27 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(tetrahydro-2H-pyran-4-yl)prop-1-yn-1- |
| | yl)pyridin-2-yl)oxy)pyrrolidine-2-carboxylic acid; |
| 28 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(tetrahydro-2H-pyran-3-yl)prop-1-yn-1- |
| | yl)pyridin-2-yl)oxy)pyrrolidine-2-carboxylic acid; |
| 29 | (2S,4S)-1-(2-(Difluoromethyl)benzofuro[3,2-d]pyrimidin-4-yl)-4-((5-((4- |
| | hydroxytetrahydro-2H-pyran-4-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 30 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(4-(tetrahydro-2H-pyran-2-yl)but-1-yn-1- |
| | yl)pyridin-2-yl)oxy)pyrrolidine-2-carboxylic acid; |
| 31 | (2S,4S)-1-(2-(Difluoromethyl)benzofuro[3,2-d]pyrimidin-4-yl)-4-((5-((3,3- |
| | dimethyl-1,4-dioxan-2-yl)ethynyl)-3-((S)-9-methyl-2,5-dioxa-8-azaspiro[3.5]nonan- |
| | 8-yl)pyridin-2-yl)oxy)pyrrolidine-2-carboxylic acid; |
| 32 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((1-methylpiperidin-4-yl)ethynyl)pyridin-2- |
| | yl)oxy)pyrrolidine-2-carboxylic acid; |
| 33 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(piperidin-1-yl)prop-1-yn-1-yl)pyridin-2- |
| | yl)oxy)pyrrolidine-2-carboxylic acid; |
| 34 | (2S,4S)-1-(2-(Difluoromethyl)benzofuro[3,2-d]pyrimidin-4-yl)-4-((5-((1-methyl-6- |
| | oxo-1,6-dihydropyridin-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 35 | (2S,4S)-1-(2-(Difluoromethyl)benzofuro[3,2-d]pyrimidin-4-yl)-4-((5-((1-methyl- |
| | 1H-pyrazol-4-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 36 | (2S,4S)-1-(2-(Difluoromethyl)benzofuro[3,2-d]pyrimidin-4-yl)-4-((5-((1-methyl- |
| | 1H-pyrazol-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 37 | (2S,4S)-1-(2-(Difluoromethyl)-7-fluorobenzofuro[3,2-d]pyrimidin-4-yl)-4-((5-((1- |
| | methyl-1H-pyrazol-3-yl)ethynyl)-3-((R*)-9-methyl-2,5-dioxa-8-azaspiro[3.5]nonan- |
| | 8-yl)pyridin-2-yl)oxy)pyrrolidine-2-carboxylic acid; |
| 38 | 2S,4S)-1-(2-(Difluoromethyl)-7-fluorobenzofuro[3,2-d]pyrimidin-4-yl)-4-((5-((1- |
| | methyl-1H-pyrazol-3-yl)ethynyl)-3-((S*)-9-methyl-2,5-dioxa-8-azaspiro[3.5]nonan- |
| | 8-yl)pyridin-2-yl)oxy)pyrrolidine-2-carboxylic acid; |
| 39 | (2S,4S)-1-(2-(Difluoromethyl)-9-fluorobenzofuro[3,2-d]pyrimidin-4-yl)-4-((5-((1- |
| | methyl-1H-pyrazol-3-yl)ethynyl)-3-((R*)-9-methyl-2,5-dioxa-8-azaspiro[3.5]nonan- |
| | 8-yl)pyridin-2-yl)oxy)pyrrolidine-2-carboxylic acid; |
| 40 | (2S,4S)-1-(2-(Difluoromethyl)-9-fluorobenzofuro[3,2-d]pyrimidin-4-yl)-4-((5-((1- |
| | methyl-1H-pyrazol-3-yl)ethynyl)-3-((S*)-9-methyl-2,5-dioxa-8-azaspiro[3.5]nonan- |
| | 8-yl)pyridin-2-yl)oxy)pyrrolidine-2-carboxylic acid; |
| 41 | (2S,4S)-1-(2-(Difluoromethyl)-6-methylbenzofuro[3,2-d]pyrimidin-4-yl)-4-((5-((1- |
| | methyl-1H-pyrazol-3-yl)ethynyl)-3-((R*)-9-methyl-2,5-dioxa-8-azaspiro[3.5]nonan- |
| | 8-yl)pyridin-2-yl)oxy)pyrrolidine-2-carboxylic acid; |
| 42 | (2S,4S)-1-(2-(Difluoromethyl)-6-methylbenzofuro[3,2-d]pyrimidin-4-yl)-4-((5-((1- |
| | methyl-1H-pyrazol-3-yl)ethynyl)-3-(S*)-9-methyl-2,5-dioxa-8-azaspiro[3.5]nonan- |
| | 8-yl)pyridin-2-yl)oxy)pyrrolidine-2-carboxylic acid; |
| 43 | (2S,4S)-1-(2-(Difluoromethyl)-6-(trifluoromethyl)benzofuro[3,2-d]pyrimidin-4-yl)- |
| | 4-((5-((1-methyl-1H-pyrazol-3-yl)ethynyl)-3-((S*)-9-methyl-2,5-dioxa-8- |
| | azaspiro[3.5]nonan-8-yl)pyridin-2-yl)oxy)pyrrolidine-2-carboxylic acid; |
| 44 | (2S,4S)-1-(2-Isopropylbenzofuro[3,2-d]pyrimidin-4-yl)-4-((5-((1-methyl-1H- |
| | pyrazol-3-yl)ethynyl)-3-((S*)-9-methyl-2,5-dioxa-8-azaspiro[3.5]nonan-8- |
| | yl)pyridin-2-yl)oxy)pyrrolidine-2-carboxylic acid; |
| 45 | (2S,4S)-1-(2-(Tert-butyl)benzofuro[3,2-d]pyrimidin-4-yl)-4-((5-((1-methyl-1H- |
| | pyrazol-3-yl)ethynyl)-3-((S*)-9-methyl-2,5-dioxa-8-azaspiro[3.5]nonan-8- |
| | yl)pyridin-2-yl)oxy)pyrrolidine-2-carboxylic acid; |
| 46 | (2S,4S)-1-(2-Methoxybenzofuro[3,2-d]pyrimidin-4-yl)-4-((5-((1-methyl-1H- |
| | pyrazol-3-yl)ethynyl)-3-((S*)-9-methyl-2,5-dioxa-8-azaspiro[3.5]nonan-8- |
| | yl)pyridin-2-yl)oxy)pyrrolidine-2-carboxylic acid; |
| 47 | (2S,4S)-1-(2-Ethoxybenzofuro[3,2-d]pyrimidin-4-yl)-4-((5-((1-methyl-1H-pyrazol- |
| | 3-yl)ethynyl)-3-((S*)-9-methyl-2,5-dioxa-8-azaspiro[3.5]nonan-8-yl)pyridin-2- |
| | yl)oxy)pyrrolidine-2-carboxylic acid; |
| 48 | (2S,4S)-1-(2-Isopropoxybenzofuro[3,2-d]pyrimidin-4-yl)-4-((5-((1-methyl-1H- |
| | pyrazol-3-yl)ethynyl)-3-((S*)-9-methyl-2,5-dioxa-8-azaspiro[3.5]nonan-8- |
| | yl)pyridin-2-yl)oxy)pyrrolidine-2-carboxylic acid; |
| 49 | (2S,4S)-1-(2-Cyclopropylbenzofuro[3,2-d]pyrimidin-4-yl)-4-((5-((1-methyl-1H- |
| | pyrazol-3-yl)ethynyl)-3-((S*)-9-methyl-2,5-dioxa-8-azaspiro[3.5]nonan-8- |
| | yl)pyridin-2-yl)oxy)pyrrolidine-2-carboxylic acid; |
| 50 | (2S,4S)-1-(2-((2,2-Dimethyl-4-oxo-3,8,11-trioxa-5-azatridecan-13- |
| | yl)oxy)benzofuro[3,2-d]pyrimidin-4-yl)-4-((5-((1-methyl-1H-pyrazol-3-yl)ethynyl)- |
| | 3-((S*)-9-methyl-2,5-dioxa-8-azaspiro[3.5]nonan-8-yl)pyridin-2-yl)oxy)pyrrolidine- |
| | 2-carboxylic acid; |
| 51 | (2S,4S)-1-(2-(Difluoromethyl)benzofuro[3,2-d]pyrimidin-4-yl)-4-((5-((1-methyl- |
| | 1H-pyrazol-5-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 52 | (2S,4S)-1-(2-(Difluoromethyl)benzofuro[3,2-d]pyrimidin-4-yl)-4-((5-((1-isopropyl- |
| | 1H-imidazol-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 53 | (2S,4S)-1-(2-(Difluoromethyl)benzofuro[3,2-d]pyrimidin-4-yl)-4-((5-((1-ethyl-1H- |
| | pyrazol-5-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 54 | (2S,4S)-1-(2-(Difluoromethyl)benzofuro[3,2-d]pyrimidin-4-yl)-4-((5-((1-isopropyl- |
| | 1H-pyrazol-5-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 55 | (2S,4S)-4-((5-((1-Cyclopropyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 56 | (2S,4S)-1-(2-(Difluoromethyl)benzofuro[3,2-d]pyrimidin-4-yl)-4-((5-((1-ethyl-1H- |
| | pyrazol-3-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 57 | (2S,4S)-1-(2-(Difluoromethyl)benzofuro[3,2-d]pyrimidin-4-yl)-4-((5-((1-isopropyl- |
| | 1H-pyrazol-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 58 | (2S,4S)-1-(2-(Difluoromethyl)benzofuro[3,2-d]pyrimidin-4-yl)-4-((5-((1,4- |
| | dimethyl-1H-pyrazol-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 59 | (2S,4S)-1-(2-(Difluoromethyl)benzofuro[3,2-d]pyrimidin-4-yl)-4-((5-((1,5- |
| | dimethyl-1H-pyrazol-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 60 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((1,3,5-trimethyl-1H-pyrazol-4- |
| | yl)ethynyl)pyridin-2-yl)oxy)pyrrolidine-2-carboxylic acid; |
| 61 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((3-methylisoxazol-5-yl)ethynyl)pyridin-2- |
| | yl)oxy)pyrrolidine-2-carboxylic acid; |
| 62 | (2S,4S)-1-(2-(Difluoromethyl)benzofuro[3,2-d]pyrimidin-4-yl)-4-((5-((3,5- |
| | dimethylisoxazol-4-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 63 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((2-methyloxazol-5-yl)ethynyl)pyridin-2- |
| | yl)oxy)pyrrolidine-2-carboxylic acid; |
| 64 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((4-methylthiazol-2-yl)ethynyl)pyridin-2- |
| | yl)oxy)pyrrolidine-2-carboxylic acid; |
| 65 | (2S,4S)-1-(2-(Difluoromethyl)benzofuro[3,2-d]pyrimidin-4-yl)-4-((5-((5- |
| | ethylthiazol-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 66 | (2S,4S)-4-((5-((2-Cyclopropylthiazol-5-yl)ethynyl)-3-((S)-9-methyl-2,5-dioxa-8- |
| | azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 67 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((2-methylthiazol-4-yl)ethynyl)pyridin-2- |
| | yl)oxy)pyrrolidine-2-carboxylic acid; |
| 68 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((3-methylisothiazol-5-yl)ethynyl)pyridin-2- |
| | yl)oxy)pyrrolidine-2-carboxylic acid; |
| 69 | (2S,4S)-1-(2-(Difluoromethyl)benzofuro[3,2-d]pyrimidin-4-yl)-4-((5-(3-(3,5- |
| | dimethyl-1H-pyrazol-1-yl)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 70 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(pyridin-2-ylethynyl)pyridin-2- |
| | yl)oxy)pyrrolidine-2-carboxylic acid; |
| 71 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(pyridin-3-ylethynyl)pyridin-2- |
| | yl)oxy)pyrrolidine-2-carboxylic acid; |
| 72 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(pyridin-4-ylethynyl)pyridin-2- |
| | yl)oxy)pyrrolidine-2-carboxylic acid; |
| 73 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((5-methylpyridin-3-yl)ethynyl)pyridin-2- |
| | yl)oxy)pyrrolidine-2-carboxylic acid; |
| 74 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((2-methylpyridin-3-yl)ethynyl)pyridin-2- |
| | yl)oxy)pyrrolidine-2-carboxylic acid; |
| 75 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((6-methylpyridin-2-yl)ethynyl)pyridin-2- |
| | yl)oxy)pyrrolidine-2-carboxylic acid; |
| 76 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((5-methylpyridin-2-yl)ethynyl)pyridin-2- |
| | yl)oxy)pyrrolidine-2-carboxylic acid; |
| 77 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((4-methylpyridin-3-yl)ethynyl)pyridin-2- |
| | yl)oxy)pyrrolidine-2-carboxylic acid; |
| 78 | (2S,4S)-1-(2-(Difluoromethyl)benzofuro[3,2-d]pyrimidin-4-yl)-4-((5-((5- |
| | methoxypyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 79 | (2S,4S)-1-(2-(Difluoromethyl)benzofuro[3,2-d]pyrimidin-4-yl)-4-((5-((3- |
| | methoxypyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 80 | (2S,4S)-1-(2-(Difluoromethyl)benzofuro[3,2-d]pyrimidin-4-yl)-4-((5-((6- |
| | methoxypyridin-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 81 | (2S,4S)-1-(2-(Difluoromethyl)benzofuro[3,2-d]pyrimidin-4-yl)-4-((5-((4- |
| | methoxypyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 82 | (2S,4S)-1-(2-(Difluoromethyl)benzofuro[3,2-d]pyrimidin-4-yl)-4-((5-((6- |
| | methoxypyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 83 | (2S,4S)-1-(2-(Difluoromethyl)benzofuro[3,2-d]pyrimidin-4-yl)-4-((5-((4- |
| | methoxypyridin-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 84 | (2S,4S)-1-(2-(Difluoromethyl)benzofuro[3,2-d]pyrimidin-4-yl)-4-((5-((5- |
| | methoxypyridin-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 85 | (2S,4S)-1-(2-(Difluoromethyl)benzofuro[3,2-d]pyrimidin-4-yl)-4-((5-((5,6- |
| | dimethylpyridin-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 86 | (2S,4S)-1-(2-(Difluoromethyl)benzofuro[3,2-d]pyrimidin-4-yl)-4-((5-((5-fluoro-6- |
| | methylpyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 87 | (2S,4S)-1-(2-(Difluoromethyl)benzofuro[3,2-d]pyrimidin-4-yl)-4-((5-((3-fluoro-6- |
| | methylpyridin-2-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 88 | (2S,4S)-1-(2-(Difluoromethyl)benzofuro[3,2-d]pyrimidin-4-yl)-4-((5-(imidazo[1,2- |
| | b]pyridazin-3-ylethynyl)-3-((S)-9-methyl-2,5-dioxa-8-azaspiro[3.5]nonan-8- |
| | yl)pyridin-2-yl)oxy)pyrrolidine-2-carboxylic acid; |
| 89 | (2S,4S)-4-((5-((1-Methyl-1H-pyrazol-5-yl)ethynyl)-3-((S)-9-methyl-2,5-dioxa-8- |
| | azaspiro[3.5]nonan-8-yl)pyridin-2-yl)oxy)-1-(2-methylbenzofuro[3,2-d]pyrimidin-4- |
| | yl)pyrrolidine-2-carboxylic acid; |
| 90 | (2S,4S)-4-((5-((1,4-Dimethyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 91 | (2S,4S)-4-((5-((1,3-Dimethyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 92 | (2S,4S)-4-((5-((1-Methyl-1H-pyrazol-3-yl)ethynyl)-3-((S)-9-methyl-2,5-dioxa-8- |
| | azaspiro[3.5]nonan-8-yl)pyridin-2-yl)oxy)-1-(2-methylbenzofuro[3,2-d]pyrimidin-4- |
| | yl)pyrrolidine-2-carboxylic acid; |
| 93 | (2S,4S)-4-((5-((1,4-Dimethyl-1H-pyrazol-3-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 94 | (2S,4S)-4-((5-((1,5-Dimethyl-1H-pyrazol-3-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 95 | (2S,4S)-4-((5-((1,5-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 96 | (2S,4S)-4-((5-((1,3-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-methylbenzofuro[3,2-d]pyrimidin- |
| | 4-yl)pyrrolidine-2-carboxylic acid; |
| 97 | (2S,4S)-4-((3-((S)-9-Methyl-2,5-dioxa-8-azaspiro[3.5]nonan-8-yl)-5-(pyridin-3- |
| | ylethynyl)pyridin-2-yl)oxy)-1-(2-methylbenzofuro[3,2-d]pyrimidin-4- |
| | yl)pyrrolidine-2-carboxylic acid; |
| 98 | (2S,4S)-4-((3-((S)-9-Methyl-2,5-dioxa-8-azaspiro[3.5]nonan-8-yl)-5-((2- |
| | methylpyridin-3-yl)ethynyl)pyridin-2-yl)oxy)-1-(2-methylbenzofuro[3,2- |
| | d]pyrimidin-4-yl)pyrrolidine-2-carboxylic acid; |
| 99 | (2S,4S)-1-(2-Ethylbenzofuro[3,2-d]pyrimidin-4-yl)-4-((5-((1-methyl-1H-pyrazol-5- |
| | yl)ethynyl)-3-((S)-9-methyl-2,5-dioxa-8-azaspiro[3.5]nonan-8-yl)pyridin-2- |
| | yl)oxy)pyrrolidine-2-carboxylic acid; |
| 100 | (2S,4S)-4-((5-((1,4-Dimethyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 101 | (2S,4S)-4-((5-((1,3-Dimethyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 102 | (2S,4S)-1-(2-Ethylbenzofuro[3,2-d]pyrimidin-4-yl)-4-((5-((1-methyl-1H-pyrazol-3- |
| | yl)ethynyl)-3-((S)-9-methyl-2,5-dioxa-8-azaspiro[3.5]nonan-8-yl)pyridin-2- |
| | yl)oxy)pyrrolidine-2-carboxylic acid; |
| 103 | (2S,4S)-4-((5-((1,4-Dimethyl-1H-pyrazol-3-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 104 | (2S,4S)-4-((5-((1,5-Dimethyl-1H-pyrazol-3-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 105 | (2S,4S)-4-((5-((1,3-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 106 | (2S,4S)-4-((5-((1,5-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-ethylbenzofuro[3,2-d]pyrimidin- |
| | 4-yl)pyrrolidine-2-carboxylic acid; |
| 107 | (2S,4S)-1-(2-Ethylbenzofuro[3,2-d]pyrimidin-4-yl)-4-((3-((S)-9-methyl-2,5-dioxa- |
| | 8-azaspiro[3.5]nonan-8-yl)-5-(pyridin-3-ylethynyl)pyridin-2-yl)oxy)pyrrolidine-2- |
| | carboxylic acid; |
| 108 | (2S,4S)-1-(2-Ethylbenzofuro[3,2-d]pyrimidin-4-yl)-4-((3-((S)-9-methyl-2,5-dioxa- |
| | 8-azaspiro[3.5]nonan-8-yl)-5-((2-methylpyridin-3-yl)ethynyl)pyridin-2- |
| | yl)oxy)pyrrolidine-2-carboxylic acid; |
| 109 | (2S,4S)-1-(2-Isopropylbenzofuro[3,2-d]pyrimidin-4-yl)-4-((5-((1-methyl-1H- |
| | pyrazol-5-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 110 | (2S,4S)-4-((5-((1-Ethyl-1H-pyrazol-5-yl)ethynyl)-3-((S)-9-methyl-2,5-dioxa-8- |
| | azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 111 | (2S,4S)-4-((5-((1-Isopropyl-1H-pyrazol-5-yl)ethynyl)-3-((S)-9-methyl-2,5-dioxa-8- |
| | azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 112 | (2S,4S)-4-((5-((1,3-Dimethyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 113 | (2S,4S)-4-((5-((1-Cyclopropyl-1H-pyrazol-5-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 114 | (2S,4S)-4-((5-((1,4-Dimethyl-1H-pyrazol-3-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 115 | (2S,4S)-1-(2-Isopropylbenzofuro[3,2-d]pyrimidin-4-yl)-4-((5-((1-methyl-1H- |
| | pyrazol-4-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 116 | (2S,4S)-4-((5-((1,3-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 117 | (2S,4S)-4-((5-((1,5-Dimethyl-1H-pyrazol-4-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 118 | (2S,4S)-1-(2-isopropylbenzofuro[3,2-d]pyrimidin-4-yl)-4-((3-(S*)-9-methyl-2,5- |
| | dioxa-8-azaspiro[3.5]nonan-8-yl)-5-((1,3,5-trimethyl-1H-pyrazol-4- |
| | yl)ethynyl)pyridin-2-yl)oxy)pyrrolidine-2-carboxylic acid; |
| 119 | (2S,4S)-1-(2-Isopropylbenzofuro[3,2-d]pyrimidin-4-yl)-4-((3-((S)-9-methyl-2,5- |
| | dioxa-8-azaspiro[3.5]nonan-8-yl)-5-((2-methyloxazol-4-yl)ethynyl)pyridin-2- |
| | yl)oxy)pyrrolidine-2-carboxylic acid; |
| 120 | (2S,4S)-1-(2-Isopropylbenzofuro[3,2-d]pyrimidin-4-yl)-4-((3-(S*)-9-methyl-2,5- |
| | dioxa-8-azaspiro[3.5]nonan-8-yl)-5-(pyridin-3-ylethynyl)pyridin-2- |
| | yl)oxy)pyrrolidine-2-carboxylic acid; |
| 121 | (2S,4S)-4-((5-((2-Ethylpyridin-3-yl)ethynyl)-3-((S)-9-methyl-2,5-dioxa-8- |
| | azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 122 | (2S,4S)-4-((5-((2-(Difluoromethyl)pyridin-3-yl)ethynyl)-3-((S)-9-methyl-2,5-dioxa- |
| | 8-azaspiro[3.5]nonan-8-yl)pyridin-2-yl)oxy)-1-(2-isopropylbenzofuro[3,2- |
| | d]pyrimidin-4-yl)pyrrolidine-2-carboxylic acid; |
| 123 | (2S,4S)-1-(2-Isopropylbenzofuro[3,2-d]pyrimidin-4-yl)-4-((3-((S)-9-methyl-2,5- |
| | dioxa-8-azaspiro[3.5]nonan-8-yl)-5-((2-(trifluoromethyl)pyridin-3- |
| | yl)ethynyl)pyridin-2-yl)oxy)pyrrolidine-2-carboxylic acid; |
| 124 | (2S,4S)-1-(2-Isopropylbenzofuro[3,2-d]pyrimidin-4-yl)-4-((3-((S)-9-methyl-2,5- |
| | dioxa-8-azaspiro[3.5]nonan-8-yl)-5-(pyridin-2-ylethynyl)pyridin-2- |
| | yl)oxy)pyrrolidine-2-carboxylic acid; |
| 125 | (2S,4S)-1-(2-Isopropylbenzofuro[3,2-d]pyrimidin-4-yl)-4-((3-((S)-9-methyl-2,5- |
| | dioxa-8-azaspiro[3.5]nonan-8-yl)-5-(pyridin-4-ylethynyl)pyridin-2- |
| | yl)oxy)pyrrolidine-2-carboxylic acid; |
| 126 | (2S,4S)-1-(2-(Difluoromethyl)benzofuro[3,2-d]pyrimidin-4-yl)-4-((5-(4- |
| | hydroxypent-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 127 | (2S,4S)-1-(2-(Difluoromethyl)benzofuro[3,2-d]pyrimidin-4-yl)-4-((5-(5- |
| | hydroxyhex-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 128 | (2S,4S)-1-(2-(Difluoromethyl)benzofuro[3,2-d]pyrimidin-4-yl)-4-((5-(3- |
| | isopropoxyprop-1-yn-1-yl)-3-((S)-9-methyl-2,5-dioxa-8-azaspiro[3.5]nonan-8- |
| | yl)pyridin-2-yl)oxy)pyrrolidine-2-carboxylic acid; |
| 129 | (2S,4S)-4-((5-((2,4-Dimethylpyridin-3-yl)ethynyl)-3-((S)-9-methyl-2,5-dioxa-8- |
| | azaspiro[3.5]nonan-8-yl)pyridin-2-yl)oxy)-1-(2-ethylbenzofuro[3,2-d]pyrimidin-4- |
| | yl)pyrrolidine-2-carboxylic acid; |
| 130 | (2S,4S)-4-((5-(3-Cyclopropylprop-1-yn-1-yl)-3-((S)-9-methyl-2,5-dioxa-8- |
| | azaspiro[3.5]nonan-8-yl)pyridin-2-yl)oxy)-1-(2-(difluoromethyl)benzofuro[3,2- |
| | d]pyrimidin-4-yl)pyrrolidine-2-carboxylic acid; |
| 131 | (2S,4S)-4-((5-(3-(3,3-Difluorocyclobutyl)prop-1-yn-1-yl)-3-((S)-9-methyl-2,5- |
| | dioxa-8-azaspiro[3.5]nonan-8-yl)pyridin-2-yl)oxy)-1-(2- |
| | (difluoromethyl)benzofuro[3,2-d]pyrimidin-4-yl)pyrrolidine-2-carboxylic acid; |
| 132 | (2S,4S)-1-(2-(Difluoromethyl)benzofuro[3,2-d]pyrimidin-4-yl)-4-((5-((1- |
| | methoxycyclopentyl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 133 | (2S,4S)-4-((5-(3-(4,4-difluorocyclohexyl)prop-1-yn-1-yl)-3-((S)-9-methyl-2,5- |
| | dioxa-8-azaspiro[3.5]nonan-8-yl)pyridin-2-yl)oxy)-1-(2- |
| | (difluoromethyl)benzofuro[3,2-d]pyrimidin-4-yl)pyrrolidine-2-carboxylic acid; |
| 134 | (2S,4S)-1-(2-(difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(oxetan-3-yl)prop-1-yn-1-yl)pyridin-2- |
| | yl)oxy)pyrrolidine-2-carboxylic acid; |
| 135 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(3-methyloxetan-3-yl)ethynyl]-2- |
| | pyridyl]oxy]pyrrolidine-2-carboxylic acid; |
| 136 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(((R*)-tetrahydrofuran-2-yl)ethynyl)pyridin- |
| | 2-yl)oxy)pyrrolidine-2-carboxylic acid; |
| 137 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(((S*)-tetrahydrofuran-2-yl)ethynyl)pyridin- |
| | 2-yl)oxy)pyrrolidine-2-carboxylic acid; |
| 138 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((2-methyltetrahydrofuran-2- |
| | yl)ethynyl)pyridin-2-yl)oxy)pyrrolidine-2-carboxylic acid; |
| 139 | (2S,4S)-1-(2-(difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(pyrrolidin-1-yl)prop-1-yn-1-yl)pyridin- |
| | 2-yl)oxy)pyrrolidine-2-carboxylic acid; |
| 140 | (2S,4S)-1-(2-(difluoromethyl)benzofuro[3,2-d]pyrimidin-4-yl)-4-((5-(3-(3,3- |
| | dimethylpyrrolidin-1-yl)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 141 | (2S,4S)-1-[2-(Difluoromethyl)benzofuro[3,2-d]pyrimidin-4-yl]-4-[[5-[2-[(2R)-1,2- |
| | dimethylpyrrolidin-2-yl]ethynyl]-3-[(9S)-9-methyl-2,5-dioxa-8-azaspiro[3.5]nonan- |
| | 8-yl]-2-pyridyl]oxy]pyrrolidine-2-carboxylic acid; |
| 142 | (2S,4S)-1-[2-(Difluoromethyl)benzofuro[3,2-d]pyrimidin-4-yl]-4-[[5-[2-[(2S)-1,2- |
| | dimethylpyrrolidin-2-yl]ethynyl]-3-[(9S)-9-methyl-2,5-dioxa-8-azaspiro[3.5]nonan- |
| | 8-yl]-2-pyridyl]oxy]pyrrolidine-2-carboxylic acid; |
| 143 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(2-oxopyrrolidin-1-yl)prop-1-yn-1- |
| | yl)pyridin-2-yl)oxy)pyrrolidine-2-carboxylic acid; |
| 144 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(2-oxooxazolidin-3-yl)prop-1-yn-1- |
| | yl)pyridin-2-yl)oxy)pyrrolidine-2-carboxylic acid; |
| 145 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((4-methyltetrahydro-2H-pyran-4- |
| | yl)ethynyl)pyridin-2-yl)oxy)pyrrolidine-2-carboxylic acid; |
| 146 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-((R*)-tetrahydro-2H-pyran-3-yl)prop-1- |
| | yn-1-yl)pyridin-2-yl)oxy)pyrrolidine-2-carboxylic acid; |
| 147 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-((S*)-tetrahydro-2H-pyran-3-yl)prop-1- |
| | yn-1-yl)pyridin-2-yl)oxy)pyrrolidine-2-carboxylic acid; |
| 148 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(tetrahydro-2H-pyran-2-yl)prop-1-yn-1- |
| | yl)pyridin-2-yl)oxy)pyrrolidine-2-carboxylic acid; |
| 149 | (2S,4S)-1-[2-(Difluoromethyl)benzofuro[3,2-d]pyrimidin-4-yl]-4-[[5-[3-(4- |
| | hydroxytetrahydropyran-4-yl)prop-1-yny]-3-[(9S)-9-methyl-2,5-dioxa-8- |
| | azaspiro[3.5]nonan-8-yl]-2-pyridyl]oxy]pyrrolidine-2-carboxylic acid; |
| 150 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(4-(tetrahydro-2H-pyran-2-yl)but-1-yn-1- |
| | yl)pyridin-2-yl)oxy)pyrrolidine-2-carboxylic acid; |
| 151 | (2S,4S)-1-(2-(difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(1-methylpiperidin-2-yl)prop-1-yn-1- |
| | yl)pyridin-2-yl)oxy)pyrrolidine-2-carboxylic acid; |
| 152 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(4-methylpiperidin-1-yl)prop-1-yn-1- |
| | yl)pyridin-2-yl)oxy)pyrrolidine-2-carboxylic acid; |
| 153 | (2S,4S)-1-[2-(Difluoromethyl)benzofuro[3,2-d]pyrimidin-4-yl]-4-[[5-[3-(4-hydroxy- |
| | 1-piperidyl)prop-1-ynyl]-3-[(9S)-9-methyl-2,5-dioxa-8-azaspiro[3.5]nonan-8-yl]-2- |
| | pyridyl]oxy]pyrrolidine-2-carboxylic acid; |
| 154 | (2S,4S)-1-[2-(Difluoromethyl)benzofuro[3,2-d]pyrimidin-4-yl]-4-[[5-[3-[(3R)-3- |
| | hydroxy-1-piperidyl]prop-1-ynyl]-3-[(9S)-9-methyl-2,5-dioxa-8- |
| | azaspiro[3.5]nonan-8-yl]-2-pyridyl]oxy]pyrrolidine-2-carboxylic acid; |
| 155 | (2S,4S)-1-[2-(Difluoromethyl)benzofuro[3,2-d]pyrimidin-4-yl]-4-[[5-[3-[(2S)-2- |
| | (hydroxymethyl)-1-piperidyl]prop-1-ynyl]-3-[(9S)-9-methyl-2,5-dioxa-8- |
| | azaspiro[3.5]nonan-8-yl]-2-pyridyl]oxy]pyrrolidine-2-carboxylic acid; |
| 156 | (2S,4S)-1-(2-(Difluoromethyl)benzofuro[3,2-d]pyrimidin-4-yl)-4-((5-(3-(4,4- |
| | difluoropiperidin-1-yl)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 157 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(1-methyl-3-piperidyl)ethynyl]-2- |
| | pyridyl]oxy]pyrrolidine-2-carboxylic acid; |
| 158 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(2-oxopiperidin-1-yl)prop-1-yn-1- |
| | yl)pyridin-2-yl)oxy)pyrrolidine-2-carboxylic acid; |
| 159 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-morpholinoprop-1-yn-1-yl)pyridin-2- |
| | yl)oxy)pyrrolidine-2-carboxylic acid; |
| 160 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(3-methylmorpholino)prop-1-yn-1- |
| | yl)pyridin-2-yl)oxy)pyrrolidine-2-carboxylic acid; |
| 161 | (2S,4S)-1-(2-(Difluoromethyl)benzofuro[3,2-d]pyrimidin-4-yl)-4-((5-(3-(2,6- |
| | dimethylmorpholino)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 162 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-(3-(4-methylpiperazin-1-yl)prop-1-yn-1- |
| | yl)pyridin-2-yl)oxy)pyrrolidine-2-carboxylic acid; |
| 163 | (2S,4S)-1-(2-(Difluoromethyl)-7-fluoro-6-methylbenzofuro[3,2-d]pyrimidin-4-yl)-4- |
| | ((5-((1-methyl-1H-pyrazol-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 164 | (2S,4S)-1-(2,6-Bis(difluoromethyl)benzofuro[3,2-d]pyrimidin-4-yl)-4-((5-((1- |
| | methyl-1H-pyrazol-3-yl)ethynyl)-3-((S*)-9-methyl-2,5-dioxa-8-azaspiro[3.5]nonan- |
| | 8-yl)pyridin-2-yl)oxy)pyrrolidine-2-carboxylic acid; |
| 165 | (2S,4S)-4-[[5-[2-(1-Cyclopropylpyrazol-3-yl)ethynyl]-3-[(9S)-9-methyl-2,5-dioxa- |
| | 8-azaspiro[3.5]nonan-8-yl]-2-pyridyl]oxy]-1-[2-(difluoromethyl)benzofuro[3,2- |
| | d]pyrimidin-4-yl]pyrrolidine-2-carboxylic acid; |
| 166 | (2S,4S)-1-[2-(Difluoromethyl)benzofuro[3,2-d]pyrimidin-4-yl]-4-[[5-[2-(5- |
| | isopropyl-1-methyl-pyrazol-4-yl)ethynyl]-3-[(9S)-9-methyl-2,5-dioxa-8- |
| | azaspiro[3.5]nonan-8-yl]-2-pyridyl]oxy]pyrrolidine-2-carboxylic acid; |
| 167 | (2S,4S)-1-(2-(Difluoromethyl)-7-fluoro-6-methylbenzofuro[3,2-d]pyrimidin-4-yl)-4- |
| | ((5-((1,3-dimethyl-1H-pyrazol-4-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 168 | (2S,4S)-1-(2-(Difluoromethyl)benzofuro[3,2-d]pyrimidin-4-yl)-4-((5-((1,3- |
| | dimethyl-1H-pyrazol-4-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 169 | (2S,4S)-4-[[5-[2-(3-Cyclopropyl-1-methyl-pyrazol-4-yl)ethynyl]-3-[(9S)-9-methyl- |
| | 2,5-dioxa-8-azaspiro[3.5]nonan-8-yl]-2-pyridyl]oxy]-1-[2- |
| | (difluoromethyl)benzofuro[3,2-d]pyrimidin-4-yl]pyrrolidine-2-carboxylic acid; |
| 170 | (2S,4S)-1-(2-(Difluoromethyl)-7-fluoro-6-methylbenzofuro[3,2-d]pyrimidin-4-yl)-4- |
| | ((5-((1,5-dimethyl-1H-pyrazol-4-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 171 | (2S,4S)-1-(2-(Difluoromethyl)benzofuro[3,2-d]pyrimidin-4-yl)-4-((5-((1,5- |
| | dimethyl-1H-pyrazol-4-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 172 | (2S,4S)-1-[2-(Difluoromethyl)benzofuro[3,2-d]pyrimidin-4-yl]-4-[[5-[2-(3- |
| | methoxy-1-methyl-pyrazol-4-yl)ethynyl]-3-[(9S)-9-methyl-2,5-dioxa-8- |
| | azaspiro[3.5]nonan-8-yl]-2-pyridyl]oxy]pyrrolidine-2-carboxylic acid; |
| 173 | (2S,4S)-1-[2-(Difluoromethyl)benzofuro[3,2-d]pyrimidin-4-yl]-4-[[5-[2-(1- |
| | isopropylpyrazol-4-yl)ethynyl]-3-[(9S)-9-methyl-2,5-dioxa-8-azaspiro[3.5]nonan-8- |
| | yl]-2-pyridyl]oxy]pyrrolidine-2-carboxylic acid; |
| 174 | (2S,4S)-1-[2-(Difluoromethyl)benzofuro[3,2-d]pyrimidin-4-yl]-4-[[5-[2-(2,4- |
| | dimethylpyrazol-3-yl)ethynyl]-3-[(9S)-9-methyl-2,5-dioxa-8-azaspiro[3.5]nonan-8- |
| | yl]-2-pyridyl]oxy]pyrrolidine-2-carboxylic acid; |
| 175 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(1-methylimidazol-4-yl)ethynyl]- |
| | 2-pyridyl]oxy]pyrrolidine-2-carboxylic acid; |
| 176 | (2S,4S)-1-[2-(Difluoromethyl)benzofuro[3,2-d]pyrimidin-4-yl]-4-[[5-[2-(2,3- |
| | dimethylimidazol-4-yl)ethynyl]-3-[(9S)-9-methyl-2,5-dioxa-8-azaspiro[3.5]nonan-8- |
| | yl]-2-pyridyl]oxy]pyrrolidine-2-carboxylic acid; |
| 177 | (2S,4S)-1-(2-(Difluoromethyl)benzofuro[3,2-d]pyrimidin-4-yl)-4-((5-(3-(4,5- |
| | dimethyl-1H-imidazol-1-yl)prop-1-yn-1-yl)-3-((S)-9-methyl-2,5-dioxa-8- |
| | azaspiro[3.5]nonan-8-yl)pyridin-2-yl)oxy)pyrrolidine-2-carboxylic acid; |
| 178 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[4-(1,2,4-triazol-1-yl)but-1-ynyl]-2- |
| | pyridyl]oxy]pyrrolidine-2-carboxylic acid; |
| 179 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(5-methyloxazol-4-yl)ethynyl]-2- |
| | pyridyl]oxy]pyrrolidine-2-carboxylic acid; |
| 180 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(2-methyloxazol-4-yl)ethynyl]-2- |
| | pyridyl]oxy]pyrrolidine-2-carboxylic acid; |
| 181 | (2S,4S)-4-[5-[2-(5-Cyclopropyloxazol-4-yl)ethynyl]-3-[(9S)-9-methyl-2,5-dioxa-8- |
| | azaspiro[3.5]nonan-8-yl]-2-pyridyl]oxy]-1-[2-(difluoromethyl)benzofuro[3,2- |
| | d]pyrimidin-4-yl]pyrrolidine-2-carboxylic acid; |
| 182 | (2S,4S)-1-(2-(Difluoromethyl)benzofuro[3,2-d]pyrimidin-4-yl)-4-((5-((3- |
| | ethylisoxazol-4-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 183 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(4-methylisoxazol-3-yl)ethynyl]- |
| | 2-pyridyl]oxy]pyrrolidine-2-carboxylic acid; |
| 184 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(3-methylisothiazol-4- |
| | yl)ethynyl]-2-pyridyl]oxy]pyrrolidine-2-carboxylic acid; |
| 185 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(4-methylthiazol-5-yl)ethynyl]-2- |
| | pyridyl]oxy]pyrrolidine-2-carboxylic acid; |
| 186 | (2S,4S)-1-[2-(Difluoromethyl)benzofuro[3,2-d]pyrimidin-4-yl]-4-[[5-[2-(2,4- |
| | dimethylthiazol-5-yl)ethynyl]-3-[(9S)-9-methyl-2,5-dioxa-8-azaspiro[3.5]nonan-8- |
| | yl]-2-pyridyl]oxy]pyrrolidine-2-carboxylic acid; |
| 187 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(6-methyl-3-pyridyl)ethynyl]-2- |
| | pyridyl]oxy]pyrrolidine-2-carboxylic acid; |
| 188 | (2S,4S)-1-(2-(Difluoromethyl)benzofuro[3,2-d]pyrimidin-4-yl)-4-((5-((2- |
| | ethylpyridin-3-yl)ethynyl)-3-((S)-9-methyl-2,5-dioxa-8-azaspiro[3.5]nonan-8- |
| | yl)pyridin-2-yl)oxy)pyrrolidine-2-carboxylic acid; |
| 189 | (2S,4S)-1-(2-(Difluoromethyl)benzofuro[3,2-d]pyrimidin-4-yl)-4-((5-((2- |
| | (difluoromethyl)pyridin-3-yl)ethynyl)-3-((S)-9-methyl-2,5-dioxa-8- |
| | azaspiro[3.5]nonan-8-yl)pyridin-2-yl)oxy)pyrrolidine-2-carboxylic acid; |
| 190 | (2S,4S)-1-(2-(Difluoromethyl)benzofuro[3,2-d]pyrimidin-4-yl)-4-((3-((S)-9-methyl- |
| | 2,5-dioxa-8-azaspiro[3.5]nonan-8-yl)-5-((2-(trifluoromethyl)pyridin-3- |
| | yl)ethynyl)pyridin-2-yl)oxy)pyrrolidine-2-carboxylic acid; |
| 191 | (2S,4S)-1-[2-(Difluoromethyl)benzofuro[3,2-d]pyrimidin-4-yl]-4-[[5-[2-(2- |
| | methoxy-3-pyridyl)ethynyl]-3-[(9S)-9-methyl-2,5-dioxa-8-azaspiro[3.5]nonan-8-yl]- |
| | 2-pyridyl]oxy]pyrrolidine-2-carboxylic acid; |
| 192 | (2S,4S)-1-[2-(Difluoromethyl)benzofuro[3,2-d]pyrimidin-4-yl]-4-[[3-[(9S)-9- |
| | methyl-2,5-dioxa-8-azaspiro[3.5]nonan-8-yl]-5-[2-(4-methyl-2-pyridyl)ethynyl]-2- |
| | pyridyl]oxy]pyrrolidine-2-carboxylic acid; |
| 193 | (2S,4S)-1-[2-(Difluoromethyl)benzofuro[3,2-d]pyrimidin-4-yl]-4-[[5-[2-(3,5- |
| | dimethyl-2-pyridyl)ethynyl]-3-[(9S)-9-methyl-2,5-dioxa-8-azaspiro[3.5]nonan-8- |
| | yl]-2-pyridyl]oxy]pyrrolidine-2-carboxylic acid; |
| 194 | (2S,4S)-1-[2-(Difluoromethyl)benzofuro[3,2-d]pyrimidin-4-yl]-4-[[5-[2-(5-fluoro-3- |
| | methyl-2-pyridyl)ethynyl]-3-[(9S)-9-methyl-2,5-dioxa-8-azaspiro[3.5]nonan-8-yl]- |
| | 2-pyridyl]oxy]pyrrolidine-2-carboxylic acid; |
| 195 | (2S,4S)-4-((3-((S*)-9-Methyl-2,5-dioxa-8-azaspiro[3.5]nonan-8-yl)-5-((1,3,5- |
| | trimethyl-1H-pyrazol-4-yl)ethynyl)pyridin-2-yl)oxy)-1-(2- |
| | (trifluoromethyl)benzofuro[3,2-d]pyrimidin-4-yl)pyrrolidine-2-carboxylic acid; |
| 196 | (2S,4S)-4-((3-((S*)-9-Methyl-2,5-dioxa-8-azaspiro[3.5]nonan-8-yl)-5-(pyridin-3- |
| | ylethynyl)pyridin-2-yl)oxy)-1-(2-(trifluoromethyl)benzofuro[3,2-d]pyrimidin-4- |
| | yl)pyrrolidine-2-carboxylic acid; |
| 197 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(1,1,1-trifluoropropan-2- |
| | yl)benzofuro[3,2-d]pyrimidin-4-yl)pyrrolidine-2-carboxylic acid; |
| 198 | (2S,4S)-1-(2-Cyclopropylbenzofuro[3,2-d]pyrimidin-4-yl)-4-((5-((1-methyl-1H- |
| | pyrazol-5-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 199 | (2S,4S)-1-(2-Cyclopropylbenzofuro[3,2-d]pyrimidin-4-yl)-4-((5-((1,4-dimethyl-1H- |
| | pyrazol-5-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 200 | (2S,4S)-1-(2-Cyclopropylbenzofuro[3,2-d]pyrimidin-4-yl)-4-((5-((1,3-dimethyl-1H- |
| | pyrazol-5-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 201 | (2S,4S)-1-(2-Cyclopropylbenzofuro[3,2-d]pyrimidin-4-yl)-4-((5-((1-methyl-1H- |
| | pyrazol-3-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 202 | (2S,4S)-1-(2-Cyclopropylbenzofuro[3,2-d]pyrimidin-4-yl)-4-((5-((1,5-dimethyl-1H- |
| | pyrazol-3-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 203 | (2S,4S)-1-(2-Cyclopropylbenzofuro[3,2-d]pyrimidin-4-yl)-4-((5-((1,4-dimethyl-1H- |
| | pyrazol-3-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 204 | (2S,4S)-1-(2-Cyclopropylbenzofuro[3,2-d]pyrimidin-4-yl)-4-((5-((1,3-dimethyl-1H- |
| | pyrazol-4-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 205 | (2S,4S)-1-(2-Cyclopropylbenzofuro[3,2-d]pyrimidin-4-yl)-4-((5-((1,5-dimethyl-1H- |
| | pyrazol-4-yl)ethynyl)-3-((S)-9-methyl-2,5-dioxa-8-azaspiro[3.5]nonan-8-yl)pyridin- |
| | 2-yl)oxy)pyrrolidine-2-carboxylic acid; |
| 206 | (2S,4S)-1-(2-Cyclopropylbenzofuro[3,2-d]pyrimidin-4-yl)-4-((3-((S)-9-methyl-2,5- |
| | dioxa-8-azaspiro[3.5]nonan-8-yl)-5-(pyridin-3-ylethynyl)pyridin-2- |
| | yl)oxy)pyrrolidine-2-carboxylic acid; |
| 207 | (2S,4S)-1-(2-Cyclopropylbenzofuro[3,2-d]pyrimidin-4-yl)-4-((3-((S)-9-methyl-2,5- |
| | dioxa-8-azaspiro[3.5]nonan-8-yl)-5-((2-methylpyridin-3-yl)ethynyl)pyridin-2- |
| | yl)oxy)pyrrolidine-2-carboxylic acid; |
| 208 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(oxetan-3-yl)benzofuro[3,2- |
| | d]pyrimidin-4-yl)pyrrolidine-2-carboxylic acid; |
| 209 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(3-methyloxetan-3- |
| | yl)benzofuro[3,2-d]pyrimidin-4-yl)pyrrolidine-2-carboxylic acid; |
| 210 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((R*)-2-methyloxetan-2- |
| | yl)benzofuro[3,2-d]pyrimidin-4-yl)pyrrolidine-2-carboxylic acid; |
| 211 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((S*)-2-methyloxetan-2- |
| | yl)benzofuro[3,2-d]pyrimidin-4-yl)pyrrolidine-2-carboxylic acid; |
| 212 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((S)-tetrahydrofuran-2- |
| | yl)benzofuro[3,2-d]pyrimidin-4-yl)pyrrolidine-2-carboxylic acid; |
| 213 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((R)-tetrahydrofuran-2- |
| | yl)benzofuro[3,2-d]pyrimidin-4-yl)pyrrolidine-2-carboxylic acid; |
| 214 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((R*)-2-methyltetrahydrofuran-2- |
| | yl)benzofuro[3,2-d]pyrimidin-4-yl)pyrrolidine-2-carboxylic acid; |
| 215 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((S*)-2-methyltetrahydrofuran-2- |
| | yl)benzofuro[3,2-d]pyrimidin-4-yl)pyrrolidine-2-carboxylic acid; |
| 216 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((S*)-tetrahydrofuran-3- |
| | yl)benzofuro[3,2-d]pyrimidin-4-yl)pyrrolidine-2-carboxylic acid; |
| 217 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-((R*)-tetrahydrofuran-3- |
| | yl)benzofuro[3,2-d]pyrimidin-4-yl)pyrrolidine-2-carboxylic acid; |
| 218 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(3-methyltetrahydrofuran-3- |
| | yl)benzofuro[3,2-d]pyrimidin-4-yl)pyrrolidine-2-carboxylic acid; |
| 219 | (2S,4S)-1-(2-(3,3-Difluoroazetidin-1-yl)benzofuro[3,2-d]pyrimidin-4-yl)-4-((5-((1- |
| | methyl-1H-pyrazol-3-yl)ethynyl)-3-((S*)-9-methyl-2,5-dioxa-8-azaspiro[3.5]nonan- |
| | 8-yl)pyridin-2-yl)oxy)pyrrolidine-2-carboxylic acid; |
| 220 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(pyrrolidin-1-yl)benzofuro[3,2- |
| | d]pyrimidin-4-yl)pyrrolidine-2-carboxylic acid; |
| 221 | (2S,4S)-1-(2-(4,4-Difluoropiperidin-1-yl)benzofuro[3,2-d]pyrimidin-4-yl)-4-((5-((1- |
| | methyl-1H-pyrazol-3-yl)ethynyl)-3-((S*)-9-methyl-2,5-dioxa-8-azaspiro[3.5]nonan- |
| | 8-yl)pyridin-2-yl)oxy)pyrrolidine-2-carboxylic acid; |
| 222 | (2S,4S)-4-((5-((1-Methyl-1H-pyrazol-3-yl)ethynyl)-3-((S*)-9-methyl-2,5-dioxa-8- |
| | azaspiro[3.5]nonan-8-yl)pyridin-2-yl)oxy)-1-(2-(4-methylpiperazin-1- |
| | yl)benzofuro[3,2-d]pyrimidin-4-yl)pyrrolidine-2-carboxylic acid; |
| 223 | (2S,4S)-1-(2-Ethylbenzofuro[3,2-d]pyrimidin-4-yl)-4-((4-((S)-9-methyl-2,5-dioxa- |
| | 8-azaspiro[3.5]nonan-8-yl)-6-(pyridin-3-ylethynyl)pyridazin-3-yl)oxy)pyrrolidine-2- |
| | carboxylic acid; |
| 224 | (2S,4S)-1-(2-Isopropylbenzofuro[3,2-d]pyrimidin-4-yl)-4-((4-((S)-9-methyl-2,5- |
| | dioxa-8-azaspiro[3.5]nonan-8-yl)-6-((1,3,5-trimethyl-1H-pyrazol-4- |
| | yl)ethynyl)pyridazin-3-yl)oxy)pyrrolidine-2-carboxylic acid; |
| 225 | (2S,4S)-1-(2-Isopropylbenzofuro[3,2-d]pyrimidin-4-yl)-4-((4-((S)-9-methyl-2,5- |
| | dioxa-8-azaspiro[3.5]nonan-8-yl)-6-(pyridin-3-ylethynyl)pyridazin-3- |
| | yl)oxy)pyrrolidine-2-carboxylic acid; |
| 226 | (2S,4S)-1-(2-Isopropylbenzofuro[3,2-d]pyrimidin-4-yl)-4-((4-((S)-9-methyl-2,5- |
| | dioxa-8-azaspiro[3.5]nonan-8-yl)-6-((2-methylpyridin-3-yl)ethynyl)pyridazin-3- |
| | yl)oxy)pyrrolidine-2-carboxylic acid; |
| 227 | (2S,4S)-1-(2-(Difluoromethyl)benzofuro[3,2-d]pyrimidin-4-yl)-4-((4-((S*)-9- |
| | methyl-2,5-dioxa-8-azaspiro[3.5]nonan-8-yl)-6-((1,3,5-trimethyl-1H-pyrazol-4- |
| | yl)ethynyl)pyridazin-3-yl)oxy)pyrrolidine-2-carboxylic acid; |
| 228 | (2S,4S)-1-(2-(Difluoromethyl)benzofuro[3,2-d]pyrimidin-4-yl)-4-((4-((S*)-9- |
| | methyl-2,5-dioxa-8-azaspiro[3.5]nonan-8-yl)-6-((2-methylpyridin-3- |
| | yl)ethynyl)pyridazin-3-yl)oxy)pyrrolidine-2-carboxylic acid; and |
| 229 | (2S,4S)-1-(2-(Difluoromethyl)-7-fluoro-6-methylbenzofuro[3,2-d]pyrimidin-4-yl)-4- |
| | ((4-((S)-9-methyl-2,5-dioxa-8-azaspiro[3.5]nonan-8-yl)-6-((2-methylpyridin-3- |
| | yl)ethynyl)pyridazin-3-yl)oxy)pyrrolidine-2-carboxylic acid; |

and pharmaceutically acceptable salts thereof.

In certain embodiments, the present disclosure is directed to one or more compounds of Formula (I) or Formula (II) independently selected from the group consisting of:
(2S,4S)-1-(2-(Difluoromethyl)benzofuro[3,2-d]pyrimidin-4-yl)-4-((5-(4-hydroxy-4-methylpent-1-yn-1-yl)-3-((S)-9-methyl-2,5-dioxa-8-azaspiro[3.5]nonan-8-yl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-(tetrahydrofuran-3-yl)prop-1-yn-1-yl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-(tetrahydro-2H-pyran-4-yl)prop-1-yn-1-yl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-(tetrahydro-2H-pyran-3-yl)prop-1-yn-1-yl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-(piperidin-1-yl)prop-1-yn-1-yl)pyridin-2-yl)oxy)pyrrolidine-2-carboxylic acid;
(2S,4S)-1-(2-(Difluoromethyl)benzofuro[3,2-d]pyrimidin-4-yl)-4-((5-((1-ethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid;
(2S,4S)-1-(2-(Difluoromethyl)benzofuro[3,2-d]pyrimidin-4-yl)-4-((5-((1,4-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid;
(2S,4S)-1-(2-(Difluoromethyl)benzofuro[3,2-d]pyrimidin-4-yl)-4-((5-((1,5-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-((1,3,5-trimethyl-1H-pyrazol-4-yl)ethynyl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid;
(2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid;
(2S,4S)-1-(2-(Difluoromethyl)benzofuro[3,2-d]pyrimidin-4-yl)-4-((5-((3-methoxypyridin-2-yl)ethynyl)-3-((S)-9-methyl-2,5-dioxa-8-azaspiro[3.5]nonan-8-yl)pyridin-2-yl)oxy)pyrrolidine-2-carboxylic acid; and
(2S,4S)-1-(2-(Difluoromethyl)benzofuro[3,2-d]pyrimidin-4-yl)-4-((5-((4-methoxypyridin-3-yl)ethynyl)-3-((S)-9-methyl-2,5-dioxa-8-azaspiro[3.5]nonan-8-yl)pyridin-2-yl)oxy)pyrrolidine-2-carboxylic acid;
   and pharmaceutically acceptable salts thereof.

In some embodiments, the present disclosure relates to a compound, or a pharmaceutically acceptable salt thereof, selected from the group consisting of:

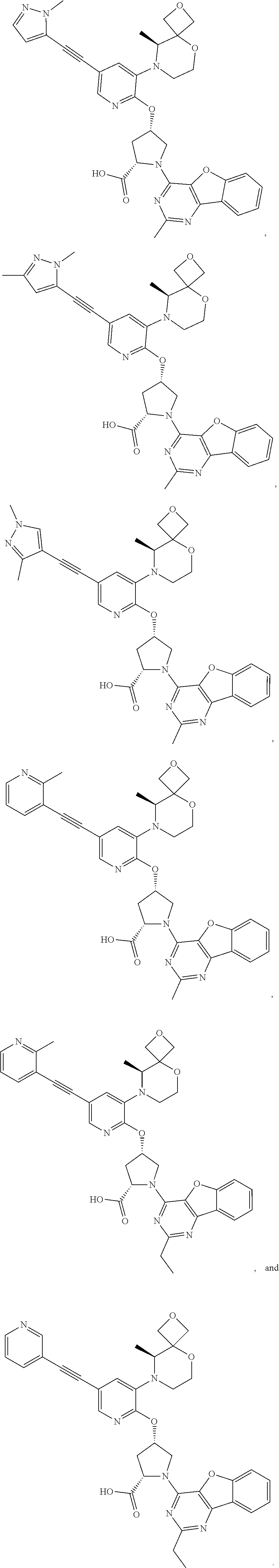

In some embodiments, the present disclosure relates to a compound selected from the group consisting of:

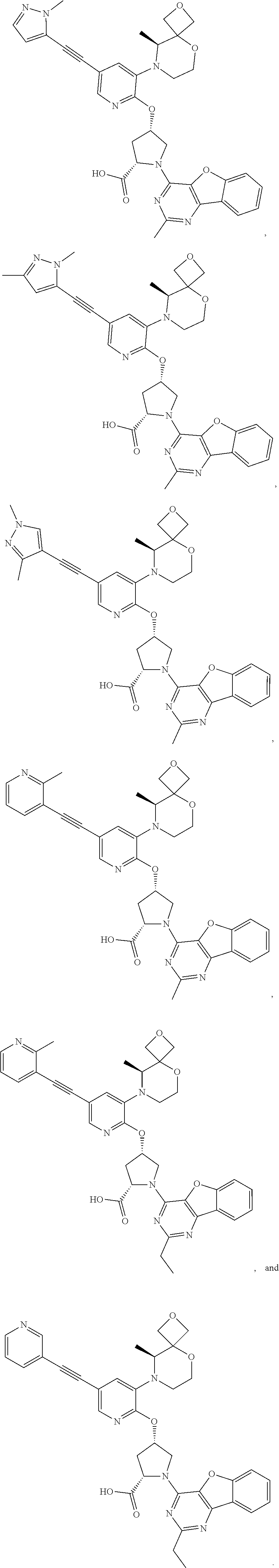

In some embodiments, the present disclosure relates to a compound, which is:

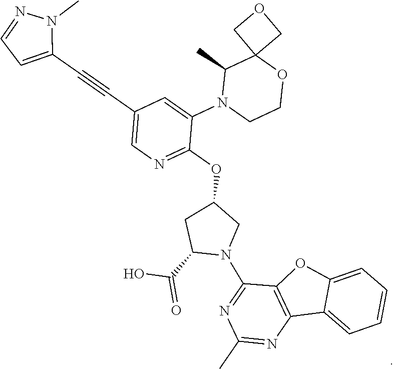 [SMILES: Cc1nc(N2C[C@@H](Oc3ncc(C#Cc4ccnn4C)cc3N3CCOC4(COC4)[C@@H]3C)C[C@H]2C(=O)O)c2oc3ccccc3c2n1]

In some embodiments, the present disclosure relates to a compound, which is:

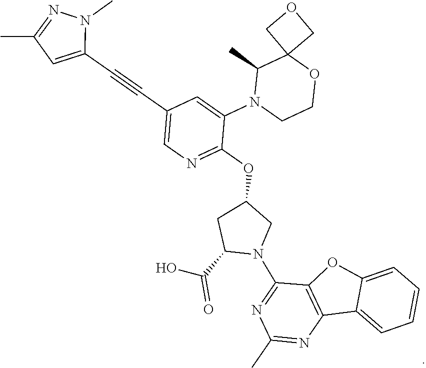 [SMILES: Cc1cc(C#Cc2cnc(O[C@H]3C[C@@H](C(=O)O)N(c4nc(C)nc5c4oc4ccccc45)C3)c(N3CCOC4(COC4)[C@@H]3C)c2)n(C)n1]

In some embodiments, the present disclosure relates to a compound, which is:

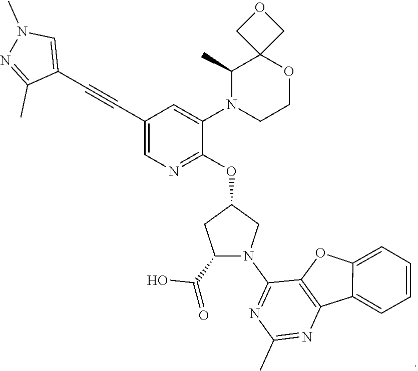 [SMILES: Cc1nc(N2C[C@@H](Oc3ncc(C#Cc4cn(C)nc4C)cc3N3CCOC4(COC4)[C@@H]3C)C[C@H]2C(=O)O)c2oc3ccccc3c2n1]

In some embodiments, the present disclosure relates to a compound, which is:

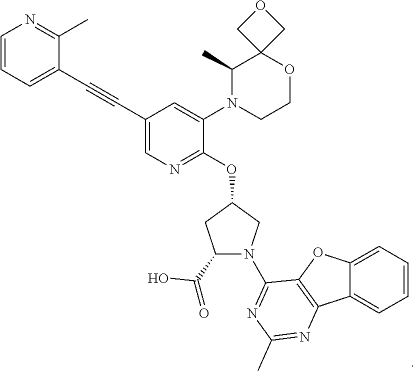 [SMILES: Cc1nc(N2C[C@@H](Oc3ncc(C#Cc4cccnc4C)cc3N3CCOC4(COC4)[C@@H]3C)C[C@H]2C(=O)O)c2oc3ccccc3c2n1]

In some embodiments, the present disclosure relates to a compound, which is:

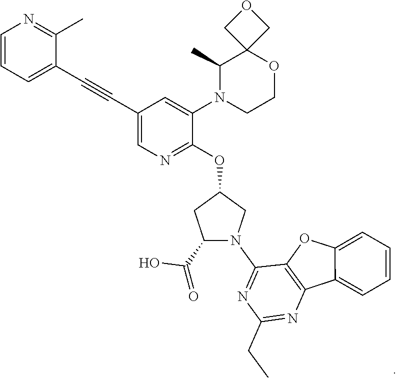 [SMILES: CCc1nc(N2C[C@@H](Oc3ncc(C#Cc4cccnc4C)cc3N3CCOC4(COC4)[C@@H]3C)C[C@H]2C(=O)O)c2oc3ccccc3c2n1]

In some embodiments, the present disclosure relates to a compound, which is:

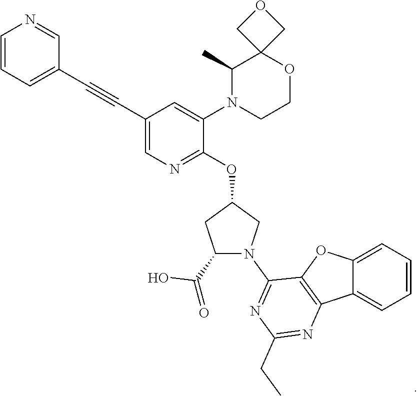 [SMILES: CCc1nc(N2C[C@@H](Oc3ncc(C#Cc4cccnc4)cc3N3CCOC4(COC4)[C@@H]3C)C[C@H]2C(=O)O)c2oc3ccccc3c2n1]

Additional embodiments of the present disclosure, include those wherein the substituents selected for one or more of the variables defined herein (i.e. R¹, R², \textcircled{A}, L¹, L², L³, L⁴, R^{a}, R^{b}, R^{c}, R^{d}, R^{e}, etc.) are independently selected to be any individual substituent or any subset of substituents selected from the complete list as defined herein.

### Methods of Treatment

Another aspect of the present disclosure includes methods of treating disease, disorder, or condition that can be treated by the inhibition of cGAS. An embodiment of the present disclosure is a method of treating a subject suffering from or diagnosed with a disease, disorder, or condition mediated by cGAS activity, comprising administering to a subject in need of such treatment a therapeutically effective amount of at least one compound selected from compounds of Formula (I) or Formula (II); and pharmaceutically acceptable salts, and stereoisomers thereof, to a subject in need thereof.

In treatment methods according to the present disclosure, a therapeutically effective amount of at least one active agent according to the present disclosure is administered to a subject suffering from or diagnosed as having such a disease, disorder, or condition. A “therapeutically effective amount” means an amount or dose sufficient to generally bring about the desired therapeutic or prophylactic benefit in subjects in need of such treatment for the designated disease, disorder, or condition. Effective amounts or doses of the active agents of the present disclosure may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. For a 70-kg human, an illustrative range for a suitable dosage amount is from about 1 to 1000 mg/day in single or multiple dosage units (e.g., BID, TID, QID or as required by modality).

Once improvement of the subject's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Subjects may, however, require intermittent treatment on a long-term basis upon any recurrence of symptoms.

In addition, the compounds of the present disclosure are envisaged for use alone, in combination with one or more of other compounds of this present disclosure, or in combination with additional active ingredients in the treatment of the conditions discussed below. The additional active ingredients may be co-administered separately with at least one compound of the present disclosure, with active agents of the present disclosure or included with such an agent in a pharmaceutical composition according to the present disclosure. In an illustrative embodiment, additional active ingredients are those that are known or discovered to be effective in the treatment of conditions, disorders, or diseases associated with the cGAS modulation, such as another cGAS inhibitor or a compound active against another target associated with the particular condition, disorder, or disease. The combination may serve to increase efficacy (e.g., by including in the combination a compound potentiating the potency or effectiveness of an agent according to the present disclosure), decrease one or more side effects, or decrease the required dose of the active agent according to the present disclosure.

When referring to inhibiting the target, an “effective amount” means an amount sufficient to affect cGAS inhibition.

In a further embodiment, the present disclosure is directed to a method of treating a subject suffering from or diagnosed with a disease, disorder, or condition associated with cGAS inhibition, comprising administering to the subject in need of such treatment a therapeutically effective amount of the active agent. In some embodiments, the active agent is a compound of the present disclosure, such as a compound of Formula (I) or a compound of Formula (II) (including compounds of Formulas (IIA), (IIB), (IIC), (IIC′), and (IID)), or a pharmaceutically acceptable salt and/or stereoisomer thereof. In some embodiments, the disease, disorder, or condition associated with cGAS inhibition is selected from autoimmune disorders including Aicardi-Goutieres Syndrome (AGS), Systemic Lupus Erythematosus (SLE), Lupus Nephritis, Scleroderma, Sjogren's Syndrome, Inflammatory Myopathies, Hidradenitis Supperativa (HS), Parkinson's Disease, Rheumatoid Arthritis, Ulcerative Colitis and Crohn's Disease.

The compounds of Formula (I) or compounds of Formula (II) (including compounds of Formulas (IIA), (IIB), (IIC), (IIC′), and (IID)) are useful in methods for treating, ameliorating and/or preventing a disease, a condition or a disorder that is affected by the inhibition of cGAS. Such methods comprise administering to a subject, including an animal, a mammal, and a human in need of such treatment, amelioration and/or prevention, a therapeutically effective amount of a compound of Formula (I) or a compound of Formula (II) (including compounds of Formulas (IIA), (IIB), (IIC), (IIC′), and (IID)), or a pharmaceutically acceptable salt thereof.

### Pharmaceutical Compositions

An additional embodiment of the present disclosure is a pharmaceutical composition comprising:
(A) therapeutically effective amount of at least one compound selected from compounds of Formula (I) or compounds of Formula (II) (including compounds of Formulas (IIA), (IIB), (IIC), (IIC′), and (ID)); and pharmaceutically acceptable salts, and stereoisomers of compounds of Formula (I) or compounds of Formula (II) (including compounds of Formulas (IIA), (IIB), (IIC), (IIC′), and (ID));
and (B) at least one pharmaceutically acceptable excipient.

An additional embodiment of the present disclosure is a pharmaceutical composition comprising a therapeutically effective amount of at least one compound in Table 1, as well as and pharmaceutically acceptable salts, and stereoisomers of compounds of Table 1, and at least one pharmaceutically acceptable excipient.

The active agents of the present disclosure are envisaged for use, alone or in combination with one or more additional active ingredients, to formulate pharmaceutical compositions of the present disclosure. A pharmaceutical composition of the present disclosure comprises a therapeutically effective amount of at least one active agent in accordance with the present disclosure.

Pharmaceutically acceptable excipients commonly used in pharmaceutical compositions are substances that are non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of such excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using pharmaceutically acceptable excipients and compounding techniques known or that become available to those of ordinary skill in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, topical administration, or oral administration. Preferably, the compositions may be formulated for oral administration.

For oral administration, the active agents of the present disclosure can be provided in the form of tablets or capsules, or as a solution, emulsion, or suspension. To prepare the oral compositions, the active agents may be formulated to yield a dosage of, e.g., for a 70-kg human, an illustrative range for a suitable dosage amount is from about 1 to 1000 mg/day in single or multiple dosage units.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Liquids for oral administration may be in the form of suspensions, solutions, emulsions, or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, compositions may be formulated for rectal administration as a suppository, enema, or foam. For parenteral use, including intravenous, intramuscular, intraperitoneal, or subcutaneous routes, the agents of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.01% to about 20% of drug to vehicle, preferably 0.1% to 10%. Another mode of administering the agents of the present disclosure may utilize a patch formulation to affect transdermal delivery.

Active agents may alternatively be administered in methods of this present disclosure by inhalation, via the nasal or oral routes, e.g., in a spray formulation also containing a suitable carrier.

### Methods of Making

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I) or a compound of Formula (II) (including compounds of Formulas (IIA), (IIB), (IIC), (IIC′), and (IID)). Reactions may be performed between the melting point and the reflux temperature of the solvent, and preferably between 0° C., and the reflux temperature of the solvent. Reactions may be heated employing conventional heating or microwave heating. Reactions may also be conducted in sealed pressure vessels above the normal reflux temperature of the solvent.

Abbreviations used in the specification, for example in the Schemes, Synthesis Examples and Biological Examples, are as listed below in Table 2.

TABLE 2
Abbreviations

| Abbreviation | Definition |
|---|---|
| Ac₂O | Acetic anhydride |
| AgOAc | Silver acetate |
| aq. | Aqueous |
| B₂Pin₂ | Bis(pinacolato)diboron |
| BINAP | 2,2′-Bis(diphenylphosphino)-1,1′-binaphthalene, (±)- |
| | BINAP, [1,1-Binaphthalene]-2,2- |
| | diylbis[diphenylphosphine] |
| BINAP Pd G4 | (Methanesulfonatato-κO)[2-(methylamino-κN)-2- |
| | biphenylyl-κC2]palladium-1,1-binaphthalene-2,2- |
| | diylbis(diphenylphosphine) |
| Boc or BOC | tert-butyloxycarbonyl |
| BOP | benzotriazol-1-yloxytris(dimethylamino)phosphonium |
| | hexafluorophosphate |
| br | broad |
| calcd. | calculated |
| CataCXium A Pd G3 | Mesylate[(di(1-adamantyl)-n-butylphosphine)-2-(2-amino- |
| | 1,1-biphenyl)]palladium(II) |
| cataCXium Pd G4 complex | [di(adamantan-1-yl)(butyl)phosphine](methanesulfonato- |
| | κO)[2-(methylamino)-2-biphenylyl]palladium |
| Celite ® | Diatomaceous Earth |
| cGAS or cGas | Cyclic GMP-AMP synthase |
| conc. | concentrated |
| CPME | Cyclopentyl methyl ether |
| Cs₂CO₃ | Cesium carbonate |
| CuI | Copper (I) iodide |
| δ | NMR chemical shift in parts per million downfield from a |
| | standard |
| d | day(s) or doublet |
| DABCO | 1,4-Diazabicyclo[2.2.2]octane |
| DAST | Diethylaminosulfur trifluoride |
| DBU | 1,8-Diazabicyclo[5.4.0]undec-7-ene |
| DCE | 1,2-dichloroethane |
| DCM | dichloromethane |
| DEA | Diethanolamine |
| DIEA or DIPEA | N,N-Diisopropylethylamine |
| DMA | Dimethylacetamide |
| DMAP | 4-(Dimethylamino)pyridine |
| DMF | dimethylformamide |
| DMSO | Dimethylsulfoxide |
| ESI | Electrospray ionization |
| Et | Ethyl |
| Et₃N | Triethylamine |
| EtOAc | Ethyl Acetate |
| EtOH | Ethanol |
| FA | Formic acid |
| g | gram(s) |
| h, hr, hrs | hour(s) |
| H₃PO₄ | Phosphoric acid |
| H₂O₂ | Hydrogen peroxide |
| HAL | Halogen atom |
| HATU | 1-[bis(dimethylamino)methylene]-1H-1,2,3-triazolo[4,5- |
| | b]pyridinium 3-oxid hexafluorophosphate |
| HOAc | Acetic acid |
| HPLC | High Pressure Liquid Chromatography |
| Hunig's base | N,N-Diisopropylethylamine |
| Hz | Hertz |
| ICl | Iodine monochloride |
| IPA or iPrOH | isopropylalcohol |
| iPrONa | Sodium isopropoxide |
| J | coupling constant (NMR spectroscopy) |
| kg | kilogram |
| L | Liter |
| KOAc | Potassium acetate |
| K₂CO₃ | Potassium carbonate |
| LiOMe | Lithium Methoxide |
| M | Molar (moles/liter) |
| Me | Methyl |
| m | Milli or multiplet |
| Me₂NH | dimethylamine |
| MeCN or ACN | Acetonitrile |
| MeI | Methyl iodide |
| MeMgBr | Methyl magnesium bromide |
| MeNH₂ | Methyl amine |
| MeOH | Methanol |
| m/z | Mass to charge ratio |
| mg | milligrams |
| MHz | Megahertz |
| min, mins | minutes |
| mL | milliliter |
| mmol | millimoles |
| mol | moles |
| MS | Mass spectrometry |
| MTBE | Methyl tert-Butyl Ether |
| MgSO₄ | Magnesium sulfate |
| Na₂SO₄ | Sodium sulfate |
| NaH | Sodium hydride |
| NaOH | Sodium hydroxide |
| NaOMe | Sodium methoxide |
| NaOEt | Sodium ethoxide |
| NaHMDS | Sodium hexamethyldisilazide |
| NBS | N-Bromosuccinimide |
| NMI | 1-methylimidazole |
| NMO | N-methylmorpholine |
| NMR | Nuclear Magnetic Resonance |
| Oxone ™ | Potassium peroxymonosulfate |
| Pet ether or PE | Petroleum ether |
| PCl₅ | Phosphorus pentachloride |
| Palladium(II)(π-cinnamyl) | Palladium(1-phenylallyl)chloride dimer |
| chloride dimer | |
| Pd(Amphos)Cl₂ | Bis(di-tert-butyl(4- |
| | dimethylaminophenyl)phosphine)dichloropalladium(II) |
| Pd(dppf)Cl₂ | [1,1′- |
| | Bis(diphenylphosphino)ferrocene]dichloropalladium(II) |
| Pd(OAc)₂ | Palladium(II) acetate |
| Pd(PPh₃)₂Cl₂ | Bis(triphenylphosphine)palladium(II) dichloride |
| Pd(PPh₃)₄ | Palladium-tetrakis(triphenylphosphine) |
| Pd₂(dba)₃ | Tris(dibenzylideneacetone)dipalladium(0) |
| POCl₃ | Phosphoryl chloride |
| PY or Py | Pyridine |
| rac-BINAP Pd G4 complex | (Methanesulfonatato-κO)[2′-(methylamino-κN)-2- |
| | biphenylyl-κC2]palladium-1,1′-binaphthalene-2,2′- |
| | diylbis(diphenylphosphine) |
| Rockphos Pd G3 | [(2-Di-tert-butylphosphino-3-methoxy-6-methyl-2′,4′,6′- |
| | triisopropyl-1,1′-biphenyl)-2-(2- |
| | aminobiphenyl)]palladium(II) methanesulfonate |
| RuPhos Pd G3 | (2-Dicyclohexylphosphino-2′,6′-diisopropoxy-1,1′- |
| | biphenyl)[2-(2′-amino-1,1′-biphenyl)]palladium(II) |
| | methanesulfonate |
| Rt or rt or r.t. | Room temperature |
| s | singlet |
| SFC | Supercritical fluid chromatography |
| Sphos | 2-Dicyclohexylphosphino-2,6-dimethoxybiphenyl |
| sulfolane | Tetrahydrothiophene 1,1-dioxide |
| T₃P | 2,4,6-tripropyl-1,3,5,2,4,6-trioxatriphosphorinane-2,4,6- |
| | trioxide |
| tBu₃P•HBF₄ | Tri-tert-butylphosphonium tetrafluoroborate |
| tBuONa | sodium t-butoxide |
| t-BuXPhos Pd G3 | tBuXPhos-Pd-G3, [(2-Di-tert-butylphosphino-2′,4′,6′- |
| | triisopropyl-1,1′-biphenyl)-2-(2′-amino-1,1′-biphenyl)] |
| | palladium(II) methanesulfonate |
| TCFH | Tetramethylchloroformamidinium hexafluorophosphate |
| TEA | triethylamine |
| TFA | trifluoroacetic acid |
| TFAA | Trifluoroacetic anhydride |
| THALES Nano H-Cube Mini | The H-Cube ® Mini Plus is a flow reactor which generates |
| Plus reactor | high-pressure hydrogen gas with the electrolysis of water, |
| | allowing catalytic hydrogenation reactions to be performed |
| | from atmospheric pressure and rt to 100 bar and 100° C. in |
| | minutes. |
| | Flow rate: 0.3-3 mL/min |
| | Maximum hydrogen production rate: 25-30 NmL/min |
| | Water reservoir capacity: 100 mL |
| | Water specifications: Deionized water with maximum |
| | conductivity of 71 nS/cm (min. resistance 14 MΩcm) |
| | Dimensions: Width: 217 mm (8.54″), Height: 200 mm |
| | (7.87″) with closed display, 315 mm (12.4″) with opened, |
| | Depth: 290 mm (11.42″) |
| | Weight: 7.3 kg (16.09 lb) |
| | Voltage: 100-240 V AC |
| | Typical catalyst amount: 0.1-0.3 g |
| | Recommended concentration: 0.01-1M |
| THF | tetrahydrofuran |
| TMS | Trimethylsilyl |
| v/v, | Volume by volume |
| w/w | Weight by weight |
| XPhos Pd G3 | (2-Dicyclohexylphosphino-2′,4′,6′-triisopropyl-1,1′- |
| | biphenyl)[2-(2′-amino-1,1′-biphenyl)]palladium(II) |
| | methanesulfonate |
| XPhos | 2-Dicyclohexylphosphino-2′,4′,6′-triisopropylbiphenyl |
| XantPhos Pd G4 | (SP-4-3)-[[5-(diphenylphosphino)-9,9-dimethyl-9H- |
| | xanthen-4-yl]diphenylphosphine-κP](methanesulfonato- |
| | κO)[2-(methylamino-κN)[1,1-biphenyl]-2-yl-κC]- |
| | Palladium |
| mmol | micromoles |
| μ | micro |
| μm | micrometer |
| μL | Microliter |
| Å | Angstrom |

### PREPARATIVE EXAMPLES

Exemplary compounds useful in methods of the present disclosure will now be described by reference to the illustrative synthetic schemes for their general preparation below and the specific examples to follow.

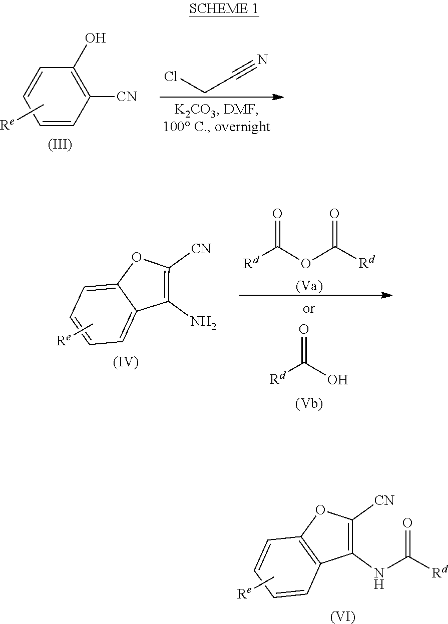

According to SCHEME 1, a commercially available or synthetically accessible compound of formula (III), where R^{e} is as defined in claim 1, is reacted with 2-chloroacetonitrile, in the presence of a suitable base such as K₂CO₃, and the like; in a suitable solvent such as DMF, and the like; at temperatures ranging from 50° C.-100° C., provides a benzofurane compound of formula (IV). Reaction of a compound of formula (IV) with a suitably substituted commercially available or synthetically accessible anhydride of formula (Va), wherein R^{d} is as defined in claim 1, with a suitable base such as pyridine, and the like: at temperatures ranging from 0° C. to room temperature; for a period of 12-24 hrs; provides a compound of formula (VI).

A compound of formula (IV) is also reacted with a compound of formula (Vb), in the presence of a suitable base such as pyridine; a suitable coupling reagent such as POCl₃, and the like; and in a suitable solvent such as DCE, at room temperature, for a period of 2 hours to provide an amide of formula (VI).

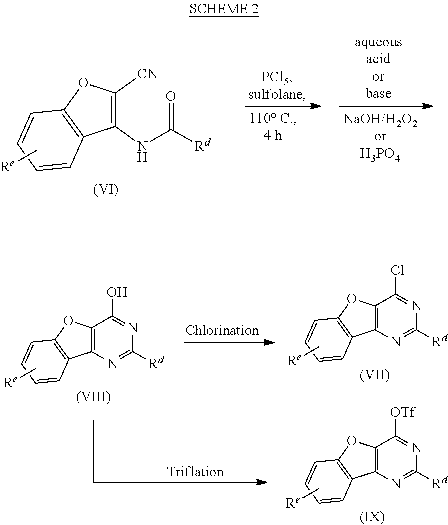

According to SCHEME 2, a commercially available or synthetically accessible compound of formula (VI), is reacted with a chlorinating agent such as PCIs, and the like; in a suitable solvent such as sulfolane; at temperatures ranging from 50° C.-150° C., for a period of 4 hours, to provide a pyrimidine compound of formula (VII).

A compound of formula (IV) is also reacted with a suitable acid, such as H₃PO₄, or a suitable base, such as NaOH/H₂O₂; and in a suitable solvent such as water, methanol, ethanol and the like, at 0° C.-100° C., for a period of 2 hours, to provide a phenol of formula (VII). Reaction of a compound of formula (VIII) with a chlorinating reagent such as POCl₃, a suitable base such as DIEA, and a suitable solvent such as 1,4-dioxane, at temperatures ranging from 0° C.-100° C., for a period of 2 hours, to provide a compound of formula (VII).

A compound of formula (VIII) is also reacted with triflic anhydride, a suitable base such as pyridine, in a suitable solvent such as DCM, at 0° C.—room temperature, for a period of 2 hours, to provide a triflate compound of formula (IX).

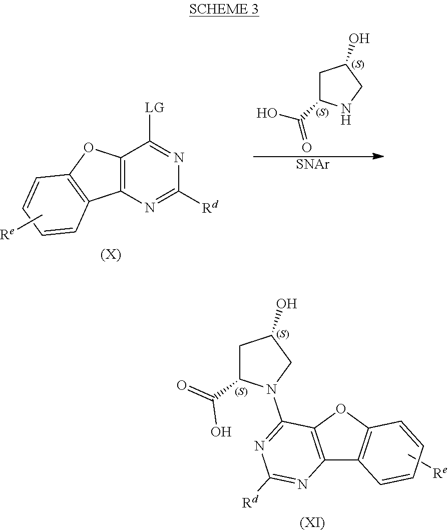

According to SCHEME 3, a commercially available or synthetically accessible compound of formula (X), is reacted via nucleophilic aromatic substitution (SNAr) with (2S,4S)-4-hydroxypyrrolidine-2-carboxylic acid in the presence of a suitable base such as sodium hydride or NaHMDS, in a suitable solvent such as DMF, at temperatures ranging from 0° C.-100° C. for period of 1-24 hours, to provide a compound of formula (XI).

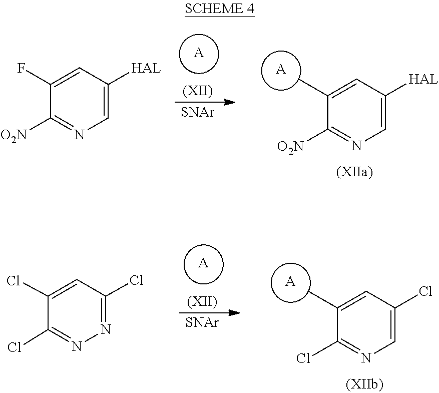

According to SCHEME 4, a commercially available or synthetically accessible halopyridine or halopyridazine is reacted via nucleophilic aromatic substitution (SNAr) with a nucleophile of formula (XII) in the presence of a suitable base such as sodium hydride or NaHMDS, in a suitable solvent such as DMF, at temperatures ranging from 0° C.-100° C. for period of 1-24 hours, to provide a compound of formula (XIIa) or formula (XIIb).

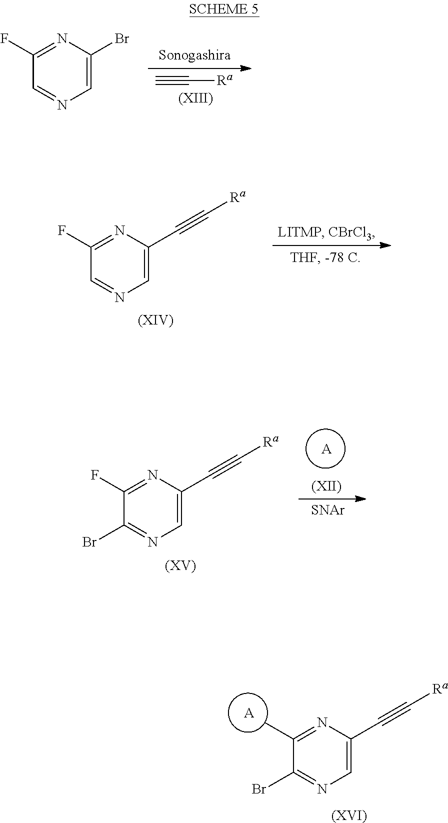

According to SCHEME 5, 2-bromo-6-fluoropyrazine may be reacted in a metal mediated cross coupling, for example, using Sonogashira conditions, with a commercially available or synthetically accessible alkyne of formula (XIII), where R^{a} is as defined in claim 1; a palladium catalyst such as PdCl₂(PPh₃)₂, and the like; a base such as triethylamine; CuI; in a suitable solvent such as DMF, ACN, and the like; to provide a compound of formula (XIV). A compound of formula (XIV) may be reacted with LiTMP; CBrCl₃; in a suitable solvent to provide a compound of formula (XV). A compound of formula (XV) may be reacted in a nucleophilic aromatic substitution (SNAr) reaction with a nucleophile of formula (XII), as previously described to provide a compound of formula (XVI).

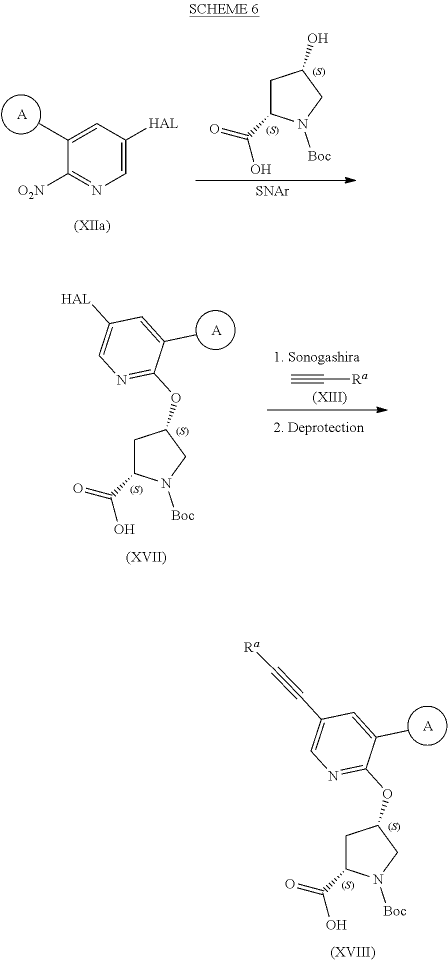

According to Scheme 6, a commercially available or synthetically accessible nitropyridine of the formula (XIIa) is reacted via nucleophilic aromatic substitution (SNAr) with (2S,4S)-4-hydroxypyrrolidine-2-carboxylic acid in the presence of a suitable base such as sodium hydride or NaHMDS, in a suitable solvent such as DMF, at temperatures ranging from 0° C.-100° C. for period of 1-24 hours, to provide a compound of formula (XVII).

A compound of formula (XVII) is also reacted via Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1-24 hours, followed by Boc deprotection with an acid such as HCl or TFA in a suitable solvent such as DCM, to provide a compound of formula (XVII).

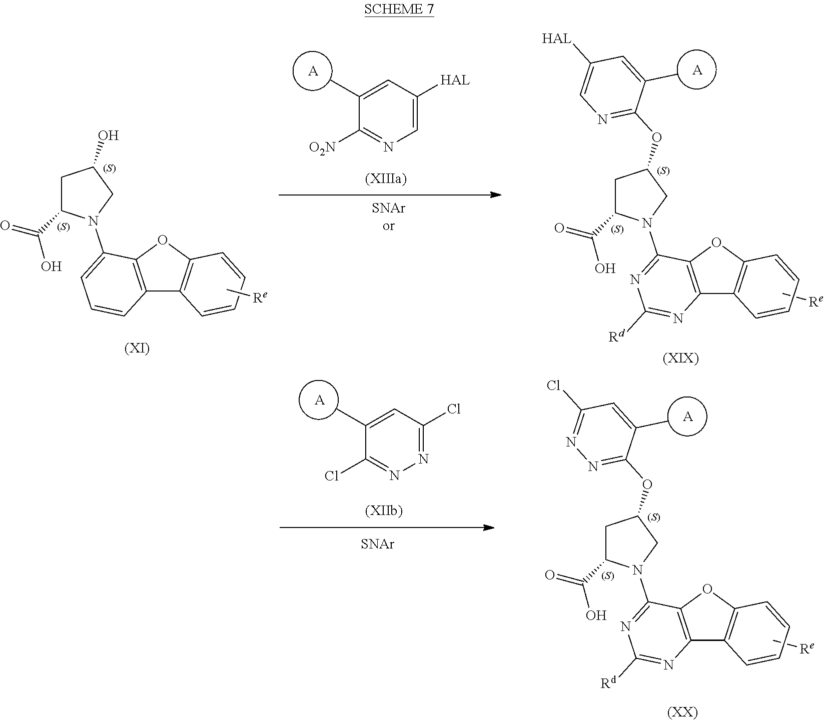

According to SCHEME 7, a compound of the formula (XI) is reacted with a nitropyridine of formula (XIIa) or a halopyridazine of formula (XIIb) via nucleophilic aromatic substitution (SNAr) using an appropriate base such as sodium hydride or NaHMDS, in an appropriate solvent such as DMF, at temperatures ranging from 0° C.-100° C. for period of 1-24 hours, to provide a compound of formula (XVII) or (XX).

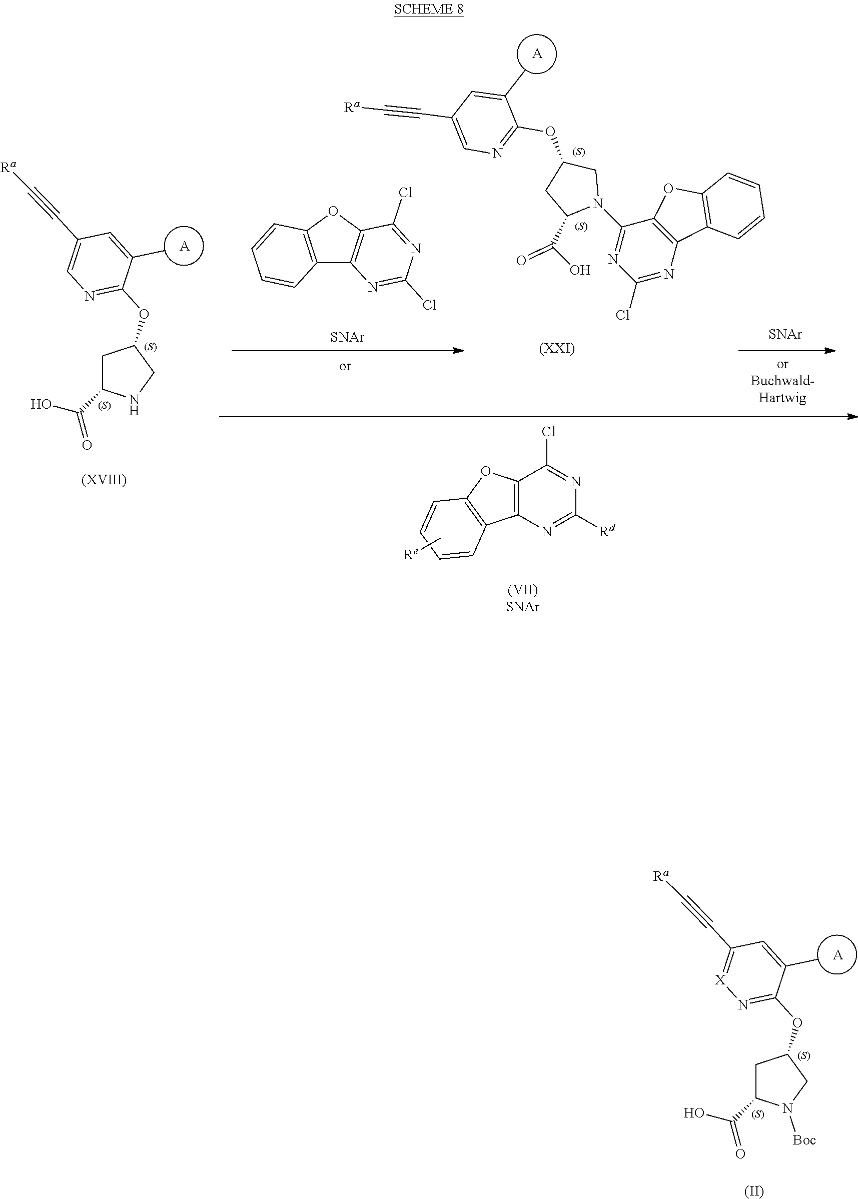

According to SCHEME 8, a compound of formula (XVIII) is reacted via nucleophilic aromatic substitution (SNAr) with 2,4-dichlorobenzofuro[3,2-d]pyrimidine using an appropriate base such as sodium hydride or NaHMDS, in an appropriate solvent such as DMF, at temperatures ranging from 0° C.-100° C. for period of 1-24 hours, to provide a compound of Formula (XXI).

A compound of formula (XXI) is also reacted via nucleophilic aromatic substitution with an appropriate alcohol or amine nucleophile using an appropriate base such as sodium hydride or NaHMDS, in an appropriate solvent such as DMF, at temperatures ranging from 0° C.-100° C. for period of 1-24 hours, to provide a compound of Formula (II).

A compound of formula (XXI) is also reacted via Buchwald coupling with an appropriate amine nucleophile using an appropriate base such as Cs₂CO₃, a catalyst system such as RuPhos Pd G3, in an appropriate solvent such as 1,4-dioxane, at temperatures ranging from 0 0° C. -100° C. for period of 1-24 hours, to provide a compound of Formula (II).

A compound of formula (XVIII) is also reacted via nucleophilic aromatic substitution (SNAr) with an electrophile or formula (VII) using an appropriate base such as sodium hydride or NaHMDS, in an appropriate solvent such as DMF, at temperatures ranging from 0° C.-100° C. for period of 1-24 hours, to provide a compound of Formula (II).

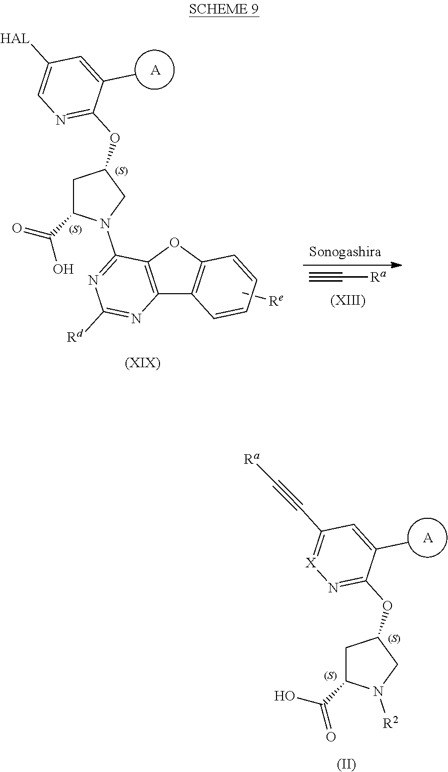

According to SCHEME 9, a halide with formula (XIX) is reacted via Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, followed by Boc deprotection with an acid such as HCl or TFA in a suitable solvent such as DCM, to provide a compound of Formula (II).

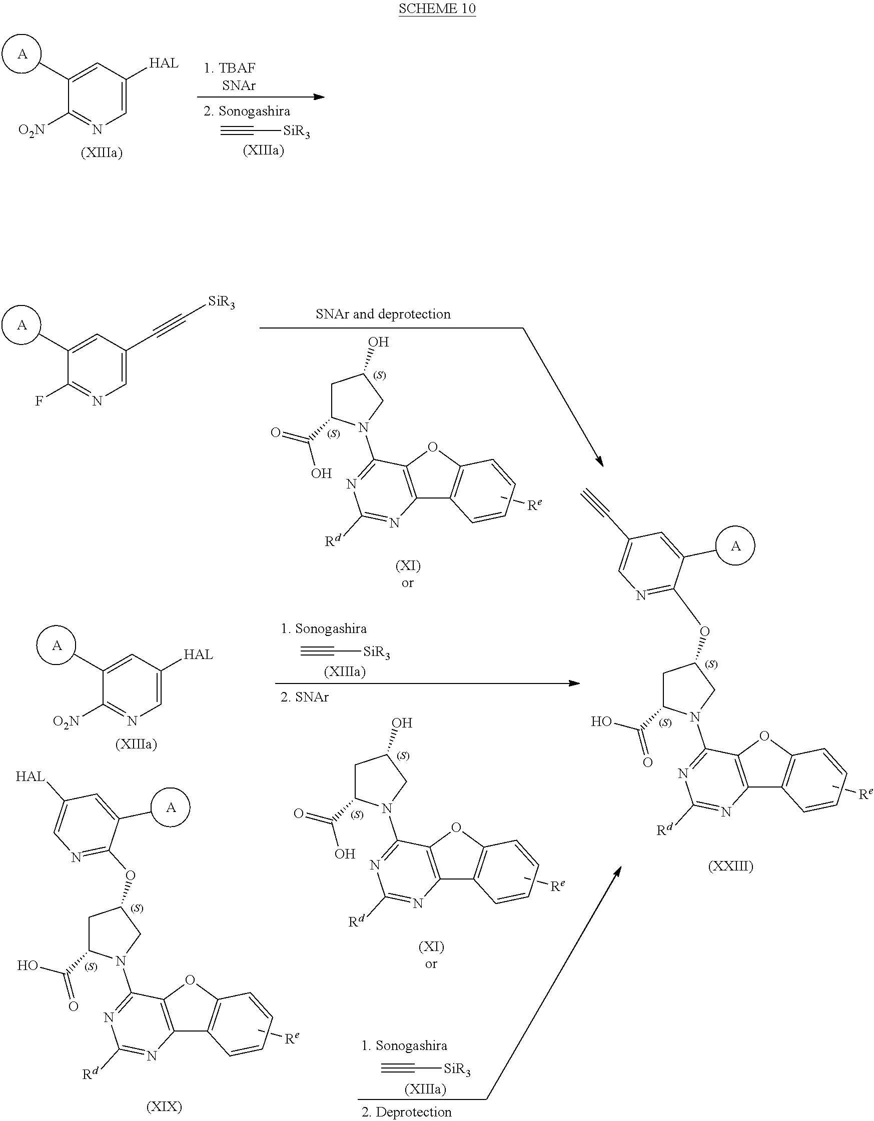

According to SCHEME 10, a compound of formula (XIIa) is reacted via nucleophilic aromatic substitution (SNAr) with tetrabutylammonium fluoride (TBAF) in a suitable solvent such as DMF, and the like; at temperatures ranging from 0° C.-100° C. for period of 1-24 hours, followed by Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, to provide a compound of formula (XXII).

A compound of formula (XXII) is also reacted via tandem nucleophilic aromatic substitution and deprotection with an alcohol of formula (XI) using an appropriate base such as sodium hydride or NaHMDS, in an appropriate solvent such as DMF, at temperatures ranging from 0° C.-100° C. for period of 1-24 hours, to provide a compound of formula (XXIII).

A compound of formula (XIIa) is reacted via Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, followed by nucleophilic aromatic substitution with an alcohol of formula (XI) using an appropriate base such as sodium hydride or NaHMDS, in an appropriate solvent such as DMF, at temperatures ranging from 0° C.-100° C. for period of 1-24 hours, to provide a compound of formula (XXIII).

A compound of formula (XIX) is reacted via Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, followed by deprotection using an appropriate base such as K₂CO₃, in an appropriate solvent such as MeOH, at temperatures ranging from 0° C.-100° C. for period of 1-24 hours, to provide a compound of formula (XXIII).

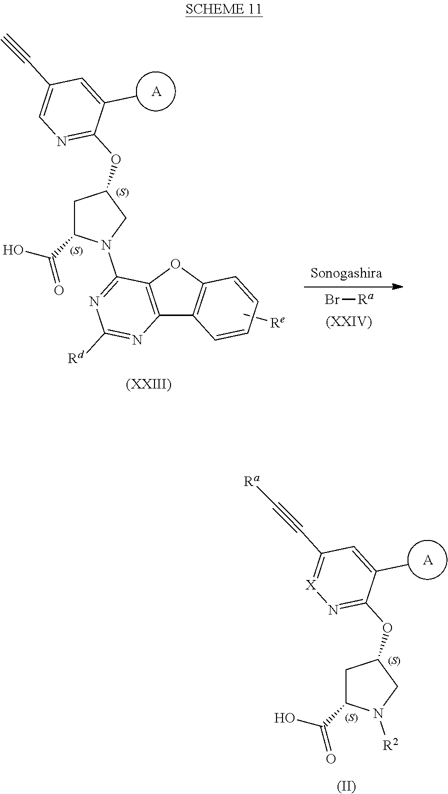

According to SCHEME 11 a compound of formula (XXIII) is reacted via Sonogashira coupling with an aryl halide, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, to provide a compound of Formula (II).

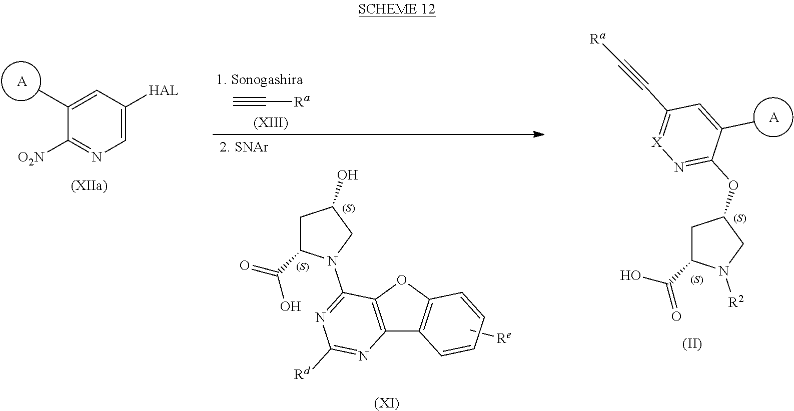

According to SCHEME 12 a compound of formula (XIIa) is reacted via Sonogashira coupling with an alkyne, an appropriate catalyst and ligand such as XPhos Pd G3 & XPhos, or Pd(Cl₂)(PPh₃)₂ and CuI, a base such as Cs₂CO₃ or DIPEA in a suitable solvent such as DMF, at temperatures ranging from rt to 100° C. for period of 1 to 24 hours, followed by nucleophilic aromatic substitution with an alcohol of formula (XI) using an appropriate base such as sodium hydride or NaHMDS, in an appropriate solvent such as DMF, at temperatures ranging from 0° C.-100° C. for period of 1-24 hours, to provide a compound of Formula (II).

Compounds of Formula (I) or compounds of Formula (II) (including compounds of Formulas (IIA), (IIB), (IIC), (IIC′), and (IID)) may be converted to their corresponding salts using methods known to one of ordinary skill in the art. For example, an amine of Formula (I) or Formula (II) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) or compounds of Formula (II) (including compounds of Formulas (IIA), (IIB), (IIC), (IIC′), and (IID)) may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### EXAMPLES

The following Examples are set forth to aid in the understanding of the present disclosure and are not intended and should not be construed to limit in any way the present disclosure set forth in the claims which follow thereafter.

Unless otherwise indicated in the examples, all temperature is expressed in Centigrade (° C.). All reactions were conducted under an inert atmosphere at ambient temperature unless otherwise noted. Unless otherwise specified, reaction solutions were stirred at room temperature under a N_{2(g)} or Ar_{(g)} atmosphere. Reagents employed without synthetic details are commercially available or made according to known methods, for example according to literature procedures. When solutions were “concentrated to dryness”, they were concentrated using a rotary evaporator under reduced pressure; when solutions were dried, they were typically dried over a drying agent such as MgSO₄ or Na₂SO₄. Where a synthesis product is listed as having been isolated as a residue, it will be understood by those skilled in the art that the term “residue” does not limit the physical state in which the product was isolated and may include, for example, a solid, an oil, a foam, a gum, a syrup, and the like.

In obtaining the compounds described in the examples below and the corresponding analytical data, the following experimental and analytical protocols were followed unless otherwise indicated.

Mass spectra: Unless otherwise noted, mass spectra were obtained on a mass spectrometer, such as Agilent series 1100 MSD using electrospray ionization (ESI) in positive mode unless otherwise indicated. Calculated mass corresponds to the exact mass.

Preparative HPLC: The compounds described in the examples may also be purified via preparative reverse-phase HPLC (e.g., Table 3). A typical HPLC chromatographic separation ranges from about 10 to about 20 minutes. Suitable solvent gradients and conditions for purification may be determined by one having skill in the art. Unless otherwise specified, where reverse-phase HPLC is used to purify a compound described in one of the examples below, the solvent used is a gradient of 10% to 80% acetonitrile in water, wherein the acetonitrile and water both contain 0.16% of either TFA or formic acid, or the acetonitrile and water have been adjusted to pH 10 with ammonium hydroxide. The following column abbreviations are also used throughout the examples.

TABLE 3

| | Column Type |
|---|---|
| C1 | Waters XSelect CSH C18, 5 μm, 19 × 100 mm |
| C2 | Waters XBridge BEH C18, 5 μm, 19 × 100 mm |
| C3 | Waters XSelect CSH C18, 5 μm, 19 × 150 mm |
| C4 | Waters XBridge BEH C18, 5 μm, 19 × 150 mm |
| C5 | Waters XSelect CSH Fluoro Phenyl, 5 μm, 19 × 100 mm |
| C6 | Waters XSelect CSH Fluoro Phenyl, 5 μm, 19 × 150 mm |
| C7 | Waters XSelect CSH C18, 5 μm, 30 × 150 mm |
| C8 | Waters XBridge BEH C18, 5 μm, 30 × 150 mm |

Normal phase flash chromatography: Unless otherwise noted, normal phase flash column chromatography (FCC) was performed on silica gel with pre-packaged silica gel columns (such as RediSep®), using suitable eluents such as ethyl acetate (EtOAc)/hexanes, ethyl acetate (EtOAc)/Petroleum ether (b.p. 60-90° C.), CH₂Cl₂/MeOH, or CH₂Cl₂/10% 2N NH₃ in MeOH.

¹H NMR: Unless otherwise noted, ¹H NMR spectra were acquired using 400 MHz spectrometers (or 300, 400 of 500 MHz spectrometers) in DMSO-d₆ solutions at temperatures ranging from room temperature to 100° C. The nuclear magnetic resonance (NMR) spectral characteristics refer to chemical shifts (6) are expressed in parts per million (ppm). Tetramethylsilane (TMS) was used as internal reference in DMSO-d₆ solutions, and residual CH₃OH peak or TMS was used as internal reference in CD₃OD solutions. Coupling constants (J) are reported in hertz (Hz). The nature of the shifts as to multiplicity is reported as s (singlet), d (doublet), t (triplet), q (quartet), dd (double doublet), dt (double triplet), m (multiplet), br (broad).

Chemical names were generated using ChemDraw Ultra 17.1 (CambridgeSoft Corp., Cambridge, MA) or OEMetaChem V1.4.0.4 (Open Eye).

### Intermediate 1: 4-Chloro-2-(difluoromethyl)benzofuro[3,2-d]pyrimidine

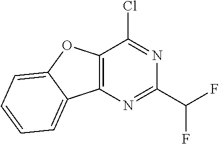 [SMILES: FC(F)c1nc(Cl)c2oc3ccccc3c2n1]

Step A. N-(2-Cyanobenzofuran-3-yl)-2,2-difluoroacetamide. To a solution of 2,2-difluoroacetic anhydride (152 g, 875 mmol) in pyridine (600 mL) was added 3-aminobenzofuran-2-carbonitrile (86.5 g, 546 mmol). The mixture was stirred at 25° C. for 12 hrs and then concentrated under reduced pressure. The residue was diluted with H₂O (1500 mL) and extracted with ethyl acetate (1000 mL×2). The organic extracts were combined, washed with brine 500 mL, dried over Na₂SO₄, filtered, concentrated under reduced pressure, and purified by flash silica gel chromatography (0-80% ethyl acetate/petroleum ether) to afford N-(2-cyanobenzofuran-3-yl)-2,2-difluoroacetamide (118 g, 465 mmol, 85% yield) as a yellow solid. LCMS (ESI): mass calcd for C₁₁H₆F₂N₂O₂, 236.0; m/z found, 237.0 [M+H]⁺. ¹H NMR (400 MHz, DMSO-d₆) δ 11.74 (s, 1H) 7.95 (d, J=8.00 Hz, 1H) 7.80-7.70 (m, 1H) 7.70-7.61 (m, 1H) 7.48 (t, J=7.60 Hz, 1H) 6.59 (t, J=53.2 Hz, 1H).

Step B. 4-Chloro-2-(difluoromethyl)benzofuro[3,2-d]pyrimidine. Reaction 1. To a solution of N-(2-cyanobenzofuran-3-yl)-2,2-difluoroacetamide (60.0 g, 254 mmol) in sulfolane (350 mL) was added PCIs (211 g, 1.02 mol). The mixture was stirred at 110° C. for 12 hrs, cooled to 0° C., and diluted with H₂O (500 mL at 0° C.). The mixture was extracted with ethyl acetate (500 mL×3). The organic extracts were washed with brine (200 mL×2), dried over Na₂SO₄, filtered, and concentrated under reduced pressure.

Reaction 2. The target compound was prepared in a manner analogous to Reaction 1. The two reaction mixture described above were combined and purified by flash silica gel chromatography (0-10% ethyl acetate/petroleum ether). The crude product was triturated with petroleum ether:ethyl acetate (20:1, 200 mL) at 25° C. for 12 hrs and then the solid was collected by filtration and dried under vacuum to afford 4-chloro-2-(difluoromethyl)benzofuro[3,2-d]pyrimidine (77.0 g, 299 mmol, 59% yield) as a light-yellow solid. LCMS (ESI): mass calcd for C₁₁H₅F₂ClN₂O, 254.0; m/z found, 254.9 [M+H]⁺. ¹H NMR (400 MHz, DMSO-d₆) δ 8.30 (d, J=7.20 Hz, 1H) 8.05-7.97 (m, 1H) 7.91 (ddd, J=8.40, 7.20, 1.20 Hz, 1H) 7.70-7.58 (m, 1H) 7.17 (t, J=54.0 Hz, 1H).

### Intermediate 2: 4-Chloro-2-(difluoromethyl)-7-fluorobenzofuro[3,2-d]pyrimidine

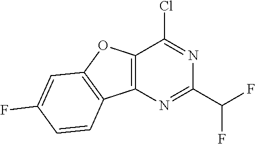 [SMILES: Fc1ccc2c(c1)oc1c(Cl)nc(C(F)F)nc12]

Step A. 3-Amino-6-fluorobenzofuran-2-carbonitrile. 2-Chloroacetonitrile (303.0 mg, 4.013 mmol), a stir bar, DMF (10.0 mL) 4-fluoro-2-hydroxybenzonitrile (500.0 mg, 3.647 mmol) and K₂CO₃ (1.54 g, 11.1 mmol) were added to a 100 mL three-necked round bottom flask, which was subsequently subjected to three cycles of vacuum and recharging with nitrogen. The yellow suspension was stirred at 100° C. overnight, then cooled to rt, filtered, and the filter cake was rinsed with EtOAc (30 mL). The filtrate was concentrated to dryness in vacuo to give a yellow solid, which was subjected to silica gel chromatography (0-30% EtOAc/pet ether) to yield 3-amino-6-fluorobenzofuran-2-carbonitrile as a yellow solid (450.0 mg, 66%). LCMS (ESI): mass calcd for C₉H₅FN₂O, 176.0; m/z found, 176.9 [M+H]⁺. ¹H NMR (400 MHz, CDCl₃) δ 7.50 (dd, J=5.2, 8.4 Hz, 1H), 7.17-7.07 (m, 2H), 4.36-4.17 (m, 2H).

Step B. N-(2-Cyano-6-fluorobenzofuran-3-yl)-2,2-difluoroacetamide. 3-Amino-6-fluorobenzofuran-2-carbonitrile (450.0 mg, 2.400 mmol), a stir bar, pyridine (4.0 mL) and 2,2-difluoroacetic anhydride (1.30 g, 7.47 mmol) were added to a 40 mL vial. The brown suspension was stirred at rt overnight, diluted with H₂O (10 mL), and extracted with EtOAc (15 mL×2). The combined extracts were dried over anhydrous Na₂SO₄, filtered, and concentrated to dryness in vacuo to give a yellow solid, which was subjected to silica gel chromatography (0-35% EtOAc/pet ether) to yield N-(2-cyano-6-fluorobenzofuran-3-yl)-2,2-difluoroacetamide as a yellow solid (850.0 mg). LCMS (ESI): mass calcd for C₁₁H₅F₃N₂O₂, 254.0; m/z found, 254.9 [M+H]⁺.

Step C. 4-Chloro-2-(difluoromethyl)-7-fluorobenzofuro[3,2-d]pyrimidine. Reaction 1. N-(2-Cyano-6-fluorobenzofuran-3-yl)-2,2-difluoroacetamide (750.0 mg crude), a stir bar, sulfolane (4.5 mL) and PCl₅ (2.10 g, 10.1 mmol) were added to a 100 mL round-bottomed flask. The yellow solution was stirred at 110° C. for 1 h, cooled to rt, and combined with another crude reaction mixtures (below).

Reaction 2. The target compound was prepared in a manner analogous to Reaction 1, except 0.3507 mmol of N-(2-Cyano-6-fluorobenzofuran-3-yl)-2,2-difluoroacetamide. The two reaction mixtures described above were combined and poured into ice water (10 mL), diluted with H₂O (15 mL), and extracted with EtOAc (20 mL×2). The combined extracts were dried over anhydrous Na₂SO₄, filtered, and concentrated to dryness in vacuo to give a yellow solid, which was subjected to silica gel chromatography (0-5% EtOAc/pet ether) to yield 4-chloro-2-(difluoromethyl)-7-fluorobenzofuro[3,2-d]pyrimidine as a yellow solid (230.0 mg, 34% over two steps). LCMS (ESI): mass calcd for C₁₁H₄ClF₃N₂O, 272.0; m/z found, 273.0 [M+H]⁺. ¹H NMR (400 MHz, CDCl₃) δ 8.24 (dd, J=5.2, 8.8 Hz, 1H), 7.41 (dd, J=2.4, 8.4 Hz, 1H), 7.31-7.24 (m, 1H), 6.72 (t, J=54.4 Hz, 1H).

### Intermediate 3: 4-Chloro-2-(difluoromethyl)-6-methylbenzofuro[3,2-d]pyrimidine

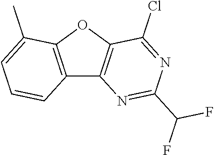 [SMILES: Cc1cccc2c1oc1c(Cl)nc(C(F)F)nc12]

Step A. 3-Amino-7-methylbenzofuran-2-carbonitrile. 2-Hydroxy-3-methylbenzonitrile (1.00 g, 7.51 mmol), a stir bar, DMF (20.0 mL), K₂CO₃ (3.10 g, 22.4 mmol), and 2-chloroacetonitrile (620.0 mg, 8.212 mmol) were added to a 250 mL three-necked round-bottomed flask, which was subsequently subjected to three cycles of vacuum and recharging with nitrogen, and the resulting yellow heterogeneous mixture was stirred at 100° C. overnight. The resulting brown suspension was cooled down to rt, filtered through a pad of diatomaceous earth and the filter cake was rinsed with EtOAc (30 mL). The filtrate was concentrated to dryness in vacuo to give a black solid, which was subjected to silica gel chromatography (0-30% EtOAc/pet ether) to yield 3-amino-7-methylbenzofuran-2-carbonitrile as a yellow solid (560.0 mg, 40%). LCMS (ESI): mass calcd for C₁₀H₈N₂O, 172.1; m/z found, 172.9 [M+H]⁺. ¹H NMR (400 MHz, CDCl₃) δ 7.35 (d, J=7.6 Hz, 1H), 7.31-7.28 (m, 1H), 7.23-7.18 (m, 1H), 4.21 (br s, 2H), 2.48 (s, 3H).

Step B. N-(2-Cyano-7-methylbenzofuran-3-yl)-2,2-difluoroacetamide. 7-Methylbenzofuran-2-carbonitrile (500.0 mg, 2.707 mmol), a stir bar, pyridine (10.0 mL), and 2,2-difluoroacetic anhydride (471.0 mg, 2.706 mmol) were added to a 100 mL three-necked round-bottomed flask at 0° C. (ice/water bath), which was subsequently subjected to three cycles of vacuum and recharging with nitrogen. The resulting yellow solution was immediately removed from ice bath, stirred at rt overnight, treated with H₂O (20 mL), and extracted with EtOAc (15 mL×2). The combined extracts were washed with brine (10 mL), dried over anhydrous Na₂SO₄, filtered, and concentrated to dryness in vacuo to give a yellow solid, which was subjected to silica gel chromatography (0-60% EtOAc/pet ether) to yield N-(2-cyano-7-methylbenzofuran-3-yl)-2,2-difluoroacetamide as a yellow solid (600.0 mg, 72%). LCMS (ESI): mass calcd for C₁₂H₈F₂N₂O₂, 250.1; m/z found, 251.1 [M+H]⁺. ¹H NMR (400 MHz, CDCl₃) δ 8.24 (br s, 1H), 7.53 (d, J=8.0 Hz, 1H), 7.39-7.35 (m, 1H), 7.33-7.28 (m, 1H), 6.18 (t, J=54.0 Hz, 1H), 2.54 (s, 3H).

Step C. 4-Chloro-2-(difluoromethyl)-6-methylbenzofuro[3,2-d]pyrimidine. N-(2-Cyano-7-methylbenzofuran-3-yl)-2,2-difluoroacetamide (580.0 mg, 1.885 mmol), a stir bar, sulfolane (10.0 mL), and PCIs (1.20 g, 5.76 mmol) were added to a 50 mL three-necked round-bottomed flask, which was subsequently subjected to three cycles of vacuum and recharging with nitrogen. The resulting yellow solution was stirred at 110° C. for 2 days, then cooled down to rt, treated with H₂O (20 mL), and extracted with EtOAc (15 mL×2). The combined extracts were washed with brine (10 mL), dried over anhydrous Na₂SO₄, filtered, and concentrated to dryness in vacuo to give a yellow solid, which was subjected to silica gel chromatography (0-60% EtOAc/petroleum ether) to yield 4-chloro-2-(difluoromethyl)-6-methylbenzofuro[3,2-d]pyrimidine as a yellow solid (370.0 mg, 68%). LCMS (ESI): mass calcd for C₁₂H₇ClF₂N₂O, 268.0; m/z found, 268.9 [M+H]⁺. ¹H NMR (400 MHz, CDCl₃) δ 8.14 (d, J=7.6 Hz, 1H), 7.61 (d, J=7.6 Hz, 1H), 7.51-7.45 (m, 1H), 6.81 (t, J=54.4 Hz, 1H), 2.69 (s, 3H).

### Intermediate 4: 4-Chloro-2-(difluoromethyl)-9-fluorobenzofuro[3,2-d]pyrimidine

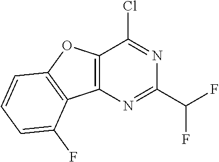 [SMILES: Fc1cccc2oc3c(Cl)nc(C(F)F)nc3c12]

Step A. 3-Amino-4-fluorobenzofuran-2-carbonitrile. 2-Chloroacetonitrile (600.0 mg, 7.947 mmol), a stir bar, DMF (20.0 mL) 2-fluoro-6-hydroxybenzonitrile (1.00 g, 7.29 mmol), K₂CO₃ (3.00 g, 21.7 mmol) were added to a 100 mL round bottom flask, which was subsequently subjected to three cycles of vacuum and recharging with nitrogen. The resulting yellow suspension was stirred for 100° C. overnight, cooled to rt, and filtered. The filter cake was rinsed with EtOAc (30 mL) and the filtrate was concentrated to dryness in vacuo to give a brown solid, which was subjected to silica gel chromatography (0-15% EtOAc/pet ether) to yield 3-amino-4-fluorobenzofuran-2-carbonitrile as a yellow solid (600.0 mg, 46%). LCMS (ESI): mass calcd for C₉H₅FN₂O, 176.0; m/z found, 177.0 [M+H]⁺. ¹H NMR (400 MHz, CDCl₃) 7.45-7.37 (m, 1H), 7.23-7.16 (m, 1H), 6.98-6.89 (m, 1H), 4.69-4.35 (m, 2H).

Step B. N-(2-cyano-4-fluorobenzofuran-3-yl)-2,2-difluoroacetamide. 2,2-Difluoroacetic anhydride (710.0 mg, 4.079 mmol), a stir bar, pyridine (15.0 mL), 3-amino-4-fluorobenzofuran-2-carbonitrile (600.0 mg, 3.385 mmol) were added to a 100 mL round bottom flask. at 0° C. (ice/water bath), which was subsequently subjected to three cycles of vacuum and recharging with nitrogen, and the resulting yellow solution was removed from ice-bath and stirred rt overnight, diluted with H₂O (30 mL) and extracted with EtOAc (30 mL×2). The combined extracts were dried over anhydrous Na₂SO₄, filtered, and concentrated to dryness in vacuo to give a yellow solid, which was subjected to silica gel chromatography (0-30% EtOAc/pet ether) to yield N-(2-cyano-4-fluorobenzofuran-3-yl)-2,2-difluoroacetamide as a yellow solid (700.0 mg, 81%). LCMS (ESI): mass calcd for C₁₁H₅F₃N₂O₂, 254.0; m/z found, 255.1 [M+H]⁺. ¹H NMR (400 MHz, CDCl₃) δ 8.45 (s, 1H), 7.58-7.50 (m, 1H), 7.41-7.35 (m, 1H), 7.14-7.06 (m, 1H), 6.18 (t, J=53.6 Hz, 1H).

Step C. 4-Chloro-2-(difluoromethyl)-9-fluorobenzofuro[3,2-d]pyrimidine. N-(2-cyano-4-fluorobenzofuran-3-yl)-2,2-difluoroacetamide (700.0 mg, 2.754 mmol), a stir bar, sulfolane (10.0 mL) and PCIs (2.65 g, 8.22 mmol) were added to a 100 mL round bottom flask. which was subsequently subjected to three cycles of vacuum and recharging with nitrogen at 0° C. (ice/water bath) to give a yellow solution. The yellow homogeneous mixture was warmed to 110° C. during 20 min and stirred at same temperature for 4 h, cooled to rt, diluted with H₂O (10 mL), and then extracted with EtOAc (20 mL×3). The combined extracts were dried over anhydrous Na₂SO₄, filtered and concentrated to dryness in vacuo to give a yellow solid, which was subjected to silica gel chromatography (0-10% EtOAc/pet ether) to yield 4-chloro-2-(difluoromethyl)-6-fluorobenzofuro[3,2-d]pyrimidine as a yellow solid (600.0 mg, 75%). LCMS (ESI): mass calcd for C₁₁H₄ClF₃N₂O, 272.0; m/z found, 273.0 [M+H]⁺. ¹H NMR (400 MHz, CDCl₃) δ 7.83-7.76 (m, 1H), 7.64-7.59 (m, 1H), 7.33-7.28 (m, 1H), 6.84 (t, J=54.4 Hz, 1H).

### Intermediate 5: 4-Chloro-2-(difluoromethyl)-6-(trifluoromethyl)benzofuro[3,2-d]pyrimidine

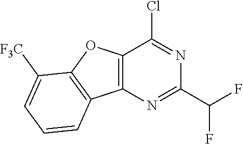 [SMILES: FC(F)c1nc(Cl)c2oc3c(C(F)(F)F)cccc3c2n1]

Step A. 2-Hydroxy-3-(trifluoromethyl)benzonitrile. 2-Fluoro-3-(trifluoromethyl)benzonitrile (1.00 g, 5.29 mmol), a stir bar, DMSO (10.0 mL), and tBuONa (1.50 g, 15.4 mmol) were added to a 40 mL vial. The black suspension was stirred at rt overnight, diluted with EtOAc (10 mL) and washed with aq LiCl (15 mL×2, 3%). The organic phase was dried over anhydrous Na₂SO₄, filtered, and concentrated to dryness in vacuo give a yellow oil, which was subjected to silica gel chromatography (0-30% EtOAc/pet ether) to yield 2-hydroxy-3-(trifluoromethyl)benzonitrile as a yellow solid (840.0 mg, 83%). LCMS (ESI): mass calcd for C₈H₄F₃NO, 187.0; m/z found, 187.8 [M+H]⁺. ¹H NMR (400 MHz, CDCl₃) δ 7.81-7.76 (m, 1H), 7.75-7.71 (m, 1H), 7.16-7.11 (m, 1H), 6.75 (br s, 1H).

Step B. 3-Amino-7-(trifluoromethyl)benzofuran-2-carbonitrile. 2-Hydroxy-3-(trifluoromethyl)benzonitrile (800.0 mg, 4.185 mmol), a stir bar, K₂CO₃ (1.70 g, 12.3 mmol), DMF (10.0 mL), and 2-chloroacetonitrile (500.0 mg, 6.623 mmol), were added to a 50 mL round bottom flask, which was subsequently subjected to three cycles of vacuum and recharging with nitrogen. The yellow suspension was stirred at 100° C. overnight, cooled to rt, filtered, and the filter cake was rinsed with EtOAc (30 mL). The filtrate was concentrated to dryness in vacuo to give a black solid, which was subjected to silica gel chromatography (0-50% EtOAc/pet ether) to yield 3-amino-7-(trifluoromethyl)benzofuran-2-carbonitrile as a yellow solid (890.0 mg, 82%). LCMS (ESI): mass calcd for C₁₀H₅F₃N₂O, 226.0; m/z found, 227.0 [M+H]⁺. ¹H NMR (400 MHz, CDCl₃) δ=7.75 (d, J=8.0 Hz, 2H), 7.43-7.37 (m, 1H) 4.37 (br s, 2H).

Step C. N-(2-Cyano-7-(trifluoromethyl)benzofuran-3-yl)-2,2-difluoroacetamide. 3-Aminobenzofuran-2-carbonitrile (880.0 mg, 3.390 mmol), a stir bar, pyridine (9.0 ml), and 2,2-difluoroacetic anhydride (1.20 g, 6.90 mmol) were added to 40 mL vial. The yellow solution was stirred at rt overnight, diluted with H₂O (15 mL), and then extracted with EtOAc (15 mL×2). The combined extracts were dried over anhydrous Na₂SO₄, filtered, and concentrated to dryness in vacuo to give a yellow solid, which was subjected to silica gel chromatography (0-30% EtOAc/pet ether) to yield N-(2-cyano-7-(trifluoromethyl)benzofuran-3-yl)-2,2-difluoroacetamide as yellow solid (670.0 mg, 60%). LCMS (ESI): mass calcd for C₁₂H₅F₅N₂O₂, 304.0; m/z found, 305.0 [M+H]⁺. ¹H NMR (400 MHz, CDCl₃) δ 8.00 (d, J=8.0 Hz, 1H), 7.84 (d, J=7.6 Hz, 1H), 7.52 (t, J=8.0 Hz, 1H), 6.20 (t, J=53.6 Hz, 1H).

Step D. 4-Chloro-2-(difluoromethyl)-6-(trifluoromethyl)benzofuro[3,2-d]pyrimidine. N-(2-Cyano-7-(trifluoromethyl)benzofuran-3-yl)-2,2-difluoroacetamide (650.0 mg, 1.957 mmol), a stir bar, sulfolane (7.0 mL), PCIs (2.40 g, 11.5 mmol) were added to a 50 mL three-necked round bottom flask, which was subsequently subjected to three cycles of vacuum and recharging with nitrogen. The yellow solution was stirred at 110° C. for 1 h, cooled to rt, diluted with H₂O (20 mL), and then extracted with EtOAc (15 mL×2). The combined extracts were washed with brine (10 mL), dried over anhydrous Na₂SO₄, filtered, and concentrated to dryness in vacuo to give a yellow solid, which was subjected to silica gel chromatography (0-30% EtOAc/pet ether) to yield 4-chloro-2-(difluoromethyl)-6-(trifluoromethyl)benzofuro[3,2-d]pyrimidine as a yellow solid (500.0 mg, 70%). LCMS (ESI): mass calcd for C₁₂H₄ClF₅N₂O, 322.0; m/z found, 322.9 [M+H]⁺. ¹H NMR (400 MHz, CDCl₃) δ 8.54 (d, J=7.6 Hz, 1H), 8.07 (d, J=7.6 Hz, 1H), 7.71 (t, J=8.0 Hz, 1H), 6.83 (t, J=54.4 Hz, 1H).

### Intermediate 6: 4-Chloro-2-cyclopropylbenzofuro[3,2-d]pyrimidine

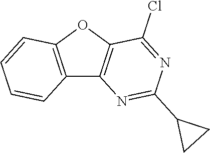 [SMILES: Clc1nc(C2CC2)nc2c1oc1ccccc12]

Step A. N-(2-Cyanobenzofuran-3-yl)cyclopropanecarboxamide. Reaction 1. Cyclopropanecarboxylic acid (775.0 mg, 9.002 mmol), a stir bar, DCE (8.0 mL), 3-aminobenzofuran-2-carbonitrile (500.0 mg, 3.161 mmol), pyridine (2.00 g, 25.3 mmol), POCl₃ (975.0 mg, 6.359 mmol) were added to a 50 mL round-bottomed flask to give a brown suspension, which was stirred at rt for 2 h to give a black solution and combined with another crude reaction mixtures (below).

Reaction 2. The target compound was prepared in a manner analogous to Reaction 1, except using 0.6323 mmol of 3-aminobenzofuran-2-carbonitrile. The two reaction mixtures described above was combined and poured into ice-water (15 mL) and extracted with EtOAc (30 mL×3). The combined extracts were dried over anhydrous Na₂SO₄, filtered and concentrated to dryness in vacuo to give a brown solid, which was subjected to silica gel chromatography (0-20% EtOAc/pet ether) to yield N-(2-cyanobenzofuran-3-yl)cyclopropanecarboxamide as a yellow solid (410.0 mg, 47%). LCMS (ESI): mass calcd for C₁₃H₁₀N₂O₂, 226.1; m/z found, 226.9 [M+H]⁺. ¹H NMR (400 MHz, DMSO-d₆) δ 11.04 (br s, 1H), 8.05 (d, J=8.0 Hz, 1H), 7.69-7.58 (m, 2H), 7.48-7.41 (m, 1H), 2.09-2.00 (m, 1H), 0.98-0.90 (m, 4H).

Step B. 4-Chloro-2-cyclopropylbenzofuro[3,2-d]pyrimidine. N-(2-Cyanobenzofuran-3-yl)cyclopropanecarboxamide (400.0 mg, 1.725 mmol), sulfolane (8.0 mL), a stir bar, PCIs (1.70 g, 8.16 mmol) were added to a 100 mL round bottom flask which was subsequently subjected to three cycles of vacuum and recharging with nitrogen at 0° C. (ice/water bath). The resulting yellow homogeneous mixture was warmed to 110° C. during 20 min and stirred at same temperature for 2 h, cooled down to rt, diluted with H₂O (20 mL) and extracted with EtOAc (15 mL×3). The combined extracts were dried over anhydrous Na₂SO₄, filtered and concentrated to dryness in vacuo to give a yellow solid, which was subjected to silica gel chromatography (0-10% EtOAc/pet ether) to yield 4-chloro-2-cyclopropylbenzofuro[3,2-d]pyrimidine as a yellow solid (250.0 mg, 59%). LCMS (ESI): mass calcd for C₁₃H₉ClN₂O, 244.0; m/z found, 245.0 [M+H]⁺. ¹H NMR (400 MHz, CDCl₃) δ 8.22 (d, J=7.6 Hz, 1H), 7.75-7.66 (m, 2H), 7.53-7.46 (m, 1H), 2.46-2.38 (m, 1H), 1.30-1.24 (m, 2H), 1.18-1.10 (m, 2H).

### Intermediate 7: 4-Chloro-2-isopropylbenzofuro[3,2-d]pyrimidine

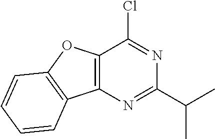 [SMILES: CC(C)c1nc(Cl)c2oc3ccccc3c2n1]

Step A. N-(2-Cyanobenzofuran-3-yl)isobutyramide. 3-Aminobenzofuran-2-carbonitrile (1.00 g, 6.32 mmol), a stir bar, pyridine (8.0 mL), and isobutyric anhydride (2.00 g, 12.6 mmol) were added to a 40 mL vial. The black suspension was stirred at 100° C. overnight, cooled to rt, diluted with H₂O (15 mL), and then extracted with EtOAc (20 mL×2). The combined extracts were dried over anhydrous Na₂SO₄, filtered, and concentrated to dryness in vacuo give a yellow solid, which was subjected to silica gel chromatography (0-35% EtOAc/pet ether) to yield N-(2-cyanobenzofuran-3-yl)isobutyramide as a yellow solid (1.10 g, 73%). LCMS (ESI): mass calcd for C₁₃H₁₂N₂O₂, 228.1; m/z found, 228.9 [M+H]⁺. ¹H NMR (400 MHz, CDCl₃) δ 7.74 (d, J=8.0 Hz, 1H), 7.71-7.61 (m, 1H), 7.55-7.44 (m, 2H), 7.37-7.31 (m, 1H), 2.79-2.66 (m, 1H), 1.35 (s, 3H), 1.33 (s, 3H).

Step B. 4-Chloro-2-isopropylbenzofuro[3,2-d]pyrimidine. N-(2-Cyanobenzofuran-3-yl)isobutyramide (1.00 g, 4.22 mmol), a stir bar, sulfolane (10.0 mL), and PCIs (5.30 g, 25.5 mmol) were added to a 100 mL three-necked round bottom flask, which was subsequently subjected to three cycles of vacuum and recharging with nitrogen. The yellow solution was stirred at 110° C. for 1 h, cooled to rt, diluted with H₂O (20 mL), and then extracted with EtOAc (15 mL×2). The combined extracts were washed with brine (10 mL), dried over anhydrous Na₂SO₄, filtered, and concentrated to dryness in vacuo to give a yellow solid, which was subjected to silica gel chromatography (0-30% EtOAc/pet ether) and followed by prep-TLC (pet ether/EtOAc=10/1) to yield 4-chloro-2-isopropylbenzofuro[3,2-d]pyrimidine as a yellow solid (65.0 mg, 6%). LCMS (ESI): mass calcd for C₁₃H₁₁ClN₂O, 246.1; m/z found, 247.1 [M+H]⁺. ¹H NMR (400 MHz, CDCl₃) δ 8.27 (d, J=8.0 Hz, 1H), 7.76-7.69 (m, 2H), 7.55-7.49 (m, 1H), 3.46-3.34 (m, 1H), 1.46 (s, 3H), 1.44 (s, 3H).

### Intermediate 8: 2-(tert-Butyl)-4-chlorobenzofuro[3,2-d]pyrimidine

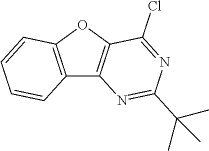 [SMILES: CC(C)(C)c1nc(Cl)c2oc3ccccc3c2n1]

Step A. N-(2-Cyanobenzofuran-3-yl)pivalamide. 3-Aminobenzofuran-2-carbonitrile (500.0 mg, 3.161 mmol), pivalic anhydride (1.80 g, 9.67 mmol), a stir bar and pyridine (10.0 mL) were added to a 100 mL round bottom flask at 0° C. (ice/water bath) to give a yellow solution, which was subsequently subjected to three cycles of vacuum and recharging with nitrogen, warmed to 100° C. during 20 min and stirred at same temperature for 2 days. The yellow homogeneous mixture was cooled to rt, diluted with H₂O (20 mL), extracted with EtOAc (20 mL×2), and the combined extracts were concentrated to dryness in vacuo to give a yellow solid, which was subjected to silica gel chromatography (0-30% EtOAc/pet ether) to yield N-(2-cyanobenzofuran-3-yl)pivalamide as a yellow solid (700.0 mg, 67%). LCMS (ESI): mass calcd for C₁₄H₁₄N₂O₂, 242.1; m/z found, 243.0 [M+H]⁺. ¹H NMR (400 MHz, CDCl₃) δ 7.71 (d, J=8.0 Hz, 1H), 7.55-7.53 (m, 1H), 7.51-7.48 (m, 1H), 7.38-7.33 (m, 1H), 1.42 (s, 9H).

Step B. 2-(tert-Butyl)-4-chlorobenzofuro[3,2-d]pyrimidine. N-(2-Cyanobenzofuran-3-yl)pivalamide (690.0 mg, 2.073 mmol), a stir bar, sulfolane (10.0 mL) and PCl₅ (1.33 g, 6.39 mmol) were added to a 100 mL round bottom flask at 0° C., which was subsequently subjected to three cycles of vacuum and recharging with nitrogen. The yellow solution was warmed to 110° C. during 20 min and stirred at same temperature for 4 h. The yellow homogeneous mixture was cooled to rt, diluted with H₂O (20 mL), extracted with EtOAc (15 mL×3), and the combined extracts were concentrated to dryness in vacuo to give a yellow solid, which was subjected to silica gel chromatography (0-15% EtOAc/pet ether) to yield 2-(tert-butyl)-4-chlorobenzofuro[3,2-d]pyrimidine as a yellow solid (60.0 mg, 11%). LCMS (ESI): mass calcd for C₁₄H₁ClN₂O, 260.1; m/z found, 261.1 [M+H]⁺. ¹H NMR (400 MHz, CDCl₃) δ 8.28-8.25 (m, 1H), 7.73-7.71 (m, 1H), 7.71 (d, J=0.8 Hz, 1H), 7.53-7.48 (m, 1H), 1.52 (s, 9H).

### Intermediate 9: (2S,4S)-1-(2-(Difluoromethyl)benzofuro[3,2-d]pyrimidin-4-yl)-4-hydroxypyrrolidine-2-carboxylic acid

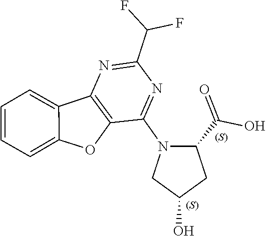

A mixture of 4-chloro-2-(difluoromethyl)benzofuro[3,2-d]pyrimidine (Chemical names were generated using ChemDraw Ultra 17.1 (CambridgeSoft Corp., Cambridge, MA) or OEMetaChem V1.4.0.4 (Open Eye).

Intermediate 1, 67.0 g, 263 mmol), (2S,4S)-4-hydroxypyrrolidine-2-carboxylic acid (34.5 g, 263 mmol), DIEA (102 g, 789 mmol, 137 mL) and DMSO (600 mL) was degassed and purged with N₂ 3 times. The mixture was then heated at 110° C. for 20 min, cooled to 0° C., and then diluted with H₂O (600 mL at 0° C.) and acidified with 4M HCl to ˜pH 5. The mixture was extracted with ethyl acetate (800 mL×5). The organic layers were combined, washed with brine (800 mL×2), dried over Na₂SO₄, filtered, concentrated under reduced pressure, and purified by flash silica gel chromatography (0-70% tetrahydrofuran/petroleum ether). The crude product was triturated with ethyl acetate (400 mL) at 25° C. for 12 hrs and then the solid was collected by filtration and dried under vacuum to afford (2S,4S)-1-(2-(difluoromethyl)benzofuro[3,2-d]pyrimidin-4-yl)-4-hydroxypyrrolidine-2-carboxylic acid (56.0 g, 160 mmol, 61% yield) as an off-white solid. LCMS (ESI): mass calcd for C₁₆H₁₃F₂N₃O₄, 349.1; m/z found, 350.0 [M+H]⁺. ¹H NMR (400 MHz, DMSO-d₆) δ 8.13 (d, J=7.60 Hz, 1H) 7.83-7.63 (m, 2H) 7.60-7.43 (m, 1H) 6.75 (t, J=54.8 Hz, 1H) 5.43-4.78 (m, 1H) 4.49 (s, 1H) 4.37-4.01 (m, 1H) 3.88 (m, 2H) 2.61-2.51 (m, 1H) 2.23 (s, 1H).

### Intermediate 10: 8-(5-Bromo-2-nitropyridin-3-yl)-9-methyl-2,5-dioxa-8-azaspiro[3.5]nonane

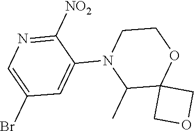 [SMILES: CC1N(c2cc(Br)cnc2[N+](=O)[O-])CCOC12COC2]

### Method A:

To a solution of 9-methyl-2,5-dioxa-8-azaspiro[3.5]nonane (22.0 g, 153 mmol) and 5-bromo-3-fluoro-2-nitropyridine (35.6 g, 161 mmol) in DMSO (200 mL) was added DIEA (59.5 g, 460 mmol, 80.2 mL). The mixture was stirred at 80° C. for 12 hrs and then cooled to rt, diluted with H₂O (200 mL) and ethyl acetate (200 mL). The mixture was extracted with ethyl acetate (200 mL×3). The organic layers were combined. washed with brine (200 mL), dried with Na₂SO₄, filtered, concentrated under reduced pressure, and purified by flash silica gel chromatography (0-25% ethyl acetate/petroleum ether) to afford 8-(5-bromo-2-nitropyridin-3-yl)-9-methyl-2,5-dioxa-8-azaspiro[3.5]nonane (35.0 g, 101 mmol, 66.1% yield) as a red solid. LCMS (ESI): mass calcd for C₁₂H₁₄BrN₃O₄, 345.0; m/z found, 345.4 [M+H]⁺. ¹H NMR (400 MHz, CDCl₃) δ 8.18 (d, J=2.00 Hz, 1H) 7.79-7.69 (m, 1H) 4.72 (d, J=7.20 Hz, 1H) 4.58 (d, J=7.20 Hz, 1H) 4.50 (d, J=7.20 Hz, 1H) 4.40 (d, J=7.20 Hz, 1H) 3.87-3.78 (m, 1H) 3.77-3.67 (m, 2H) 3.44-3.27 (m, 1H) 2.68 (dt, J=12.0, 2.80 Hz, 1H) 1.12 (d, J=6.80 Hz, 3H).

### Method B:

Reaction 1. 5-Bromo-3-fluoro-2-nitropyridine (1.50 g, 6.79 mmol), MeCN (19.0 mL), 9-methyl-2,5-dioxa-8-azaspiro[3.5]nonane (750.0 mg, 5.238 mmol), and K₂CO₃ (1.95 g, 14.1 mmol) were added to a 250 mL three-necked round-bottomed flask, the resulting yellow heterogeneous mixture was stirred at 80° C. overnight, cooled down to rt and combined with another crude reaction mixtures (below).

Reaction 2. The target compound was prepared in a manner analogous to Reaction 1 in parallel. The two reaction mixtures described above were combined and filtered, and the filter cake was rinsed with EtOAc (50 mL). The filtrate was concentrated to dryness in vacuo to give a yellow oil, which was to silica gel chromatography (0-40% EtOAc/pet ether) to yield 8-(5-bromo-2-nitropyridin-3-yl)-9-methyl-2,5-dioxa-8-azaspiro[3.5]nonane as a yellow solid (1.90 g, 46%). LCMS (ESI): mass calcd for C₁₂H₁₄BrN₃O₄, 343.0/345.0; m/z found, 344.0/345.9 [M+H]⁺. ¹H NMR (400 MHz, CDCl₃) δ 8.19 (d, J=2.0 Hz, 1H), 7.72 (d, J=2.0 Hz, 1H), 4.73 (d, J=7.2 Hz, 1H), 4.58 (d, J=7.2 Hz, 1H), 4.51 (d, J=7.2 Hz, 1H), 4.40 (d, J=7.2 Hz, 1H), 3.86-3.68 (m, 3H), 3.40-3.31 (m, 1H), 2.72-2.65 (m, 1H), 1.13 (d, J=6.8 Hz, 3H).

### Intermediate 11: (2S,4S)-4-((5-Bromo-3-((R)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid

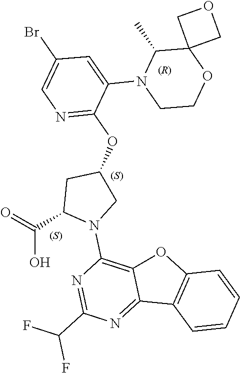

To a solution of 8-(5-bromo-2-nitropyridin-3-yl)-9-methyl-2,5-dioxa-8-azaspiro[3.5]nonane (Intermediate 10, 29.5 g, 85.8 mmol) in DMF (250 mL) was added NaH (8.59 g, 214 mmol, 60.0% purity) at 0° C. The mixture was stirred at 0° C. for 0.5 hr and then (2S,4S)-1-(2-(difluoromethyl)benzofuro[3,2-d]pyrimidin-4-yl)-4-hydroxypyrrolidine-2-carboxylic acid (Intermediate 9, 25.0 g, 71.5 mmol, 1.00 eq) was added. The mixture was warmed to rt and stirred for 12 h. The mixture was diluted with saturated aqueous NH₄Cl solution (500 mL) and H₂O (1000 mL) and then extracted with ethyl acetate (300 mL×3). The organic extracts were combined and washed with brine (200 mL), dried with Na₂SO₄, filtered, concentrated under reduced pressure, and purified by flash silica gel chromatography (0-100% ethyl acetate/petroleum ether). The product was further purified by reversed-phase HPLC (column: Phenomenex luna C18 250×80 mm×10 um; mobile phase: [water (TFA)−ACN]; gradient: 50%-80% B over 15 min) to afford (2S,4S)-4-((5-bromo-3-((R)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (first eluting isomer, 16.2 g, 24.2 mmol, 32.6% yield, off-white solid). LCMS (ESI): mass calcd for C₂₈H₂₆BrF₂N₅O₆, 647.1; m/z found, 647.8 [M+H]⁺. ¹H NMR (400 MHz, DMSO-d₆, T=80° C.) 6 8.15 (d, J=7.60 Hz, 1H) 7.90 (d, J=2.00 Hz, 1H) 7.83-7.67 (m, 2H) 7.62-7.46 (m, 1H) 7.32 (d, J=2.00 Hz, 1H) 6.77 (t, J=54.8 Hz, 1H) 5.62 (br s, 1H) 5.45-4.95 (m, 1H) 4.62 (d, J=6.40 Hz, 1H) 4.53 (d, J=6.40 Hz, 2H) 4.33 (d, J=6.80 Hz, 1H) 4.28 (br d, J=6.00 Hz, 2H) 4.11 (d, J=6.80 Hz, 1H) 3.82-3.70 (m, 1H) 3.63 (td, J=11.2, 3.20 Hz, 1H) 3.25 (td, J=11.6, 3.60 Hz, 2H) 2.74 (br d, J=12.4 Hz, 1H) 2.69-2.55 (m, 1H) 0.85 (d, J=6.80 Hz, 3H); and Intermediate 12 as the second eluting isomer.

### Intermediate 12: (2S,4S)-4-((5-Bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid

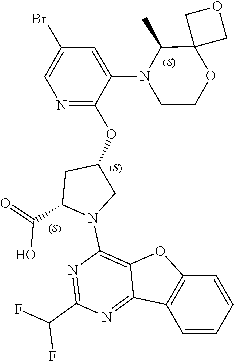

The title compound was isolated from Intermediate as the second eluting isomer (16.6 g, 33.1%), as an off-white solid. LCMS (ESI): mass calcd for C₂₈H₂₆BrF₂N₅O₆, 647.1; m/z found, 647.8 [M+H]⁺. ¹H NMR (400 MHz, DMSO-d₆, T=80° C.) 612.96-11.79 (m, 1H) 8.15 (d, J=7.60 Hz, 1H) 7.91 (d, J=2.00 Hz, 1H) 7.84-7.67 (m, 2H) 7.60-7.49 (m, 1H) 7.33 (d, J=2.00 Hz, 1H) 6.77 (t, J=54.8 Hz, 1H) 5.64 (dt, J=5.60, 2.80 Hz, 1H) 5.45-5.06 (m, 1H) 4.65 (d, J=6.80 Hz, 2H) 4.52 (d, J=6.80 Hz, 1H) 4.42 (d, J=6.80 Hz, 1H) 4.36-4.15 (m, 3H) 3.81-3.69 (m, 1H) 3.62 (td, J=11.20, 3.20 Hz, 1H) 3.37-3.18 (m, 1H) 3.01-2.87 (m, 1H) 2.74 (br d, J=12.0 Hz, 1H) 2.56 (br d, J=14.4 Hz, 1H) 0.89 (d, J=6.40 Hz, 3H).

### Intermediate 13a: (2S,4S)-4-((5-Bromo-3-((R*)-9-methyl-2,5-dioxa-8-azaspiro[3.5]nonan-8-yl)pyridin-2-yl)oxy)-1-(tert-butoxycarbonyl)pyrrolidine-2-carboxylic acid and Intermediate 13b: (2S,4S)-4-((5-bromo-3-((S*)-9-methyl-2,5-dioxa-8-azaspiro[3.5]nonan-8-yl)pyridin-2-yl)oxy)-1-(tert-butoxycarbonyl)pyrrolidine-2-carboxylic acid

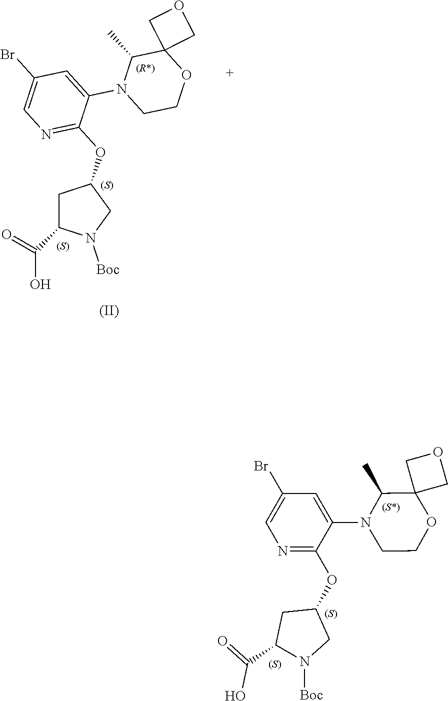

(2S,4S)-1-(tert-Butoxycarbonyl)-4-hydroxypyrrolidine-2-carboxylic acid (1.50 g, 6.49 mmol), a stir bar, DMF (28.0 mL), 8-(5-bromo-2-nitropyridin-3-yl)-9-methyl-2,5-dioxa-8-azaspiro[3.5]nonane (Intermediate 10, Method B, 1.85 g, 4.69 mmol), and NaH (814.0 mg, 20.35 mmol, 60% in mineral oil) were added to a 100 mL round-bottomed flask at 0° C. (ice/water bath), which was subsequently subjected to three cycles of vacuum and recharging with nitrogen. The resulting yellow heterogeneous mixture was immediately removed from ice bath, stirred at rt overnight, quenched with H₂O (50 mL) at 0° C., and washed with EtOAc (100 mL×2). The aqueous phase was treated with sat citric acid until pH 6 and extracted with EtOAc (100 mL×3). The combined extracts were dried over anhydrous Na₂SO₄, filtered, and concentrated to dryness in vacuo to give a yellow oil, which was subjected to silica gel chromatography (0-100% EtOAc/pet ether) to yield two products:

Intermediate 13a, 1st Eluting: (2S,4S)-4-((5-bromo-3-((R)-9-methyl-2,5-dioxa-8-azaspiro[3.5]nonan-8-yl)pyridin-2-yl)oxy)-1-(tert-butoxycarbonyl)pyrrolidine-2-carboxylic acid as a yellow solid (890.0 mg, 33%). LCMS (ESI): mass calcd for C₂₂H₃₀BrN₃O₇, 527.1/529.1; m/z found, 528.1/530.1 [M+H]⁺. ¹H NMR (400 MHz, CDCl₃) δ 7.86-7.78 (m, 1H), 7.13-7.07 (m, 1H), 5.53-5.43 (m, 1H), 4.90-4.82 (m, 2H), 4.79-4.54 (m, 3H), 4.48-4.36 (m, 1H), 4.05-3.95 (m, 1H), 3.93-3.85 (m, 1H), 3.84-3.63 (m, 2H), 3.36-3.25 (m, 1H), 2.73-2.56 (m, 2H), 2.54-2.45 (m, 1H), 1.52-1.42 (m, 9H), 0.89-0.83 (m, 3H).

Intermediate 13b, 2nd Eluting: (2S,4S)-4-((5-Bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid as a yellow sticky (1.00 g, 37%). LCMS (ESI): mass calcd for C₂₂H₃₀BrN₃O₇, 527.1/529.1; m/z found, 528.1/530.0 [M+H]⁺. ¹H NMR (400 MHz, CDCl₃) δ 7.83-7.79 (m, 1H), 7.16-7.06 (m, 1H), 5.97-5.61 (m, 1H), 5.01-4.74 (m, 2H), 4.68-4.44 (m, 3H), 4.43-4.25 (m, 1H), 4.05-3.77 (m, 2H), 3.75-3.63 (m, 2H), 3.37-3.20 (m, 1H), 2.67-2.42 (m, 3H), 1.54-1.45 (m, 9H), 0.93-0.86 (m, 3H).

### Intermediate 14: (2S,4S)-4-((5-((1-Methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid trifluoroacetate

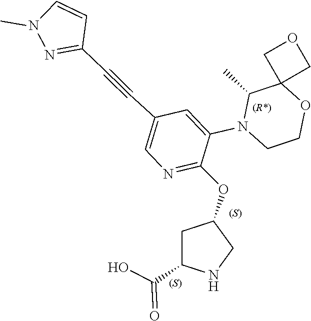

Step A. (2S,4S)-1-(tert-Butoxycarbonyl)-4-((5-((1-methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid. (2S,4S)-4-((5-Bromo-3-((R*)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 13a first eluting product, 300.0 mg, 0.5175 mmol), a stir bar, MeCN (6.0 mL), 3-ethynyl-1-methyl-1H-pyrazole (100.0 mg, 0.9423 mmol), Cs₂CO₃ (480.0 mg, 1.473 mmol), Xphos (30.0 mg, 0.0629 mmol), and Xphos Pd G3 (50.0 mg, 0.0591 mmol) were added to a nitrogen-purged 40 mL vial. The resulting yellow heterogeneous mixture was then stirred at 80° C. for 2 h, then cooled down to rt to give a black suspension, which was filtered through a pad of diatomaceous earth and the filter cake was rinsed with EtOAc (40 mL). The filtrate was concentrated to dryness in vacuo to give a brown solid, which was subjected to silica gel chromatography (0-100% EtOAc/pet ether then 0-15% MeOH/DCM) to yield (2S,4S)-1-(tert-butoxycarbonyl)-4-((5-((1-methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid as a black solid (260.0 mg, 88%). LCMS (ESI): mass calcd for C₂₈H₃₅N₅O₇, 553.3; m/z found, 554.3 [M+H]⁺. ¹H NMR (400 MHz, CDCl₃) δ 7.93-7.87 (m, 1H), 7.31-7.26 (m, 1H), 7.12-7.07 (m, 1H), 6.37 (d, J=2.4 Hz, 1H), 5.50-5.41 (m, 1H), 4.85-4.75 (m, 1H), 4.86-4.75 (m, 1H), 4.73-4.65 (m, 1H), 4.54-4.49 (m, 1H), 4.37-4.26 (m, 1H), 4.00-3.91 (m, 1H), 3.86 (s, 3H), 3.85-3.73 (m, 2H), 3.68-3.54 (m, 2H), 3.32-3.20 (m, 1H), 2.67-2.49 (m, 2H), 2.47-2.37 (m, 1H), 1.44-1.36 (m, 9H), 0.77 (d, J=6.4 Hz, 3H).

Step B. (2S,4S)-4-((5-((1-Methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid. (2S,4S)-1-(Tert-butoxycarbonyl)-4-((5-((1-methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid (260.0 mg, 0.4568 mmol), a stir bar, and TFA/DCM (4.0 mL, 1:5) were added to a 10 mL round-bottomed flask. The black solution was stirred at rt for 2 h, and concentrated to dryness in vacuo to yield (2S,4S)-4-((5-((1-methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid as a black oil (370 mg crude as TFA salt). LCMS (ESI): mass calcd for C₂₃H₂₇N₅O₅, 453.2; m/z found, 454.2 [M+H]⁺.

### Intermediate 15: (2S,4S)-4-((5-((1-Methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid trifluoracetate

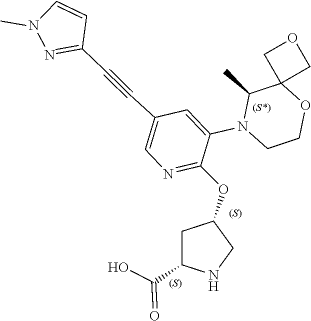

The title compound was prepared in a manner analogous to Intermediate 14, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 13b second eluting product) instead of (2S,4S)-4-((5-bromo-3-((R*)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 13a first eluting product) in Step A.

### Intermediate 16: (2S,4S)-1-(2-Chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

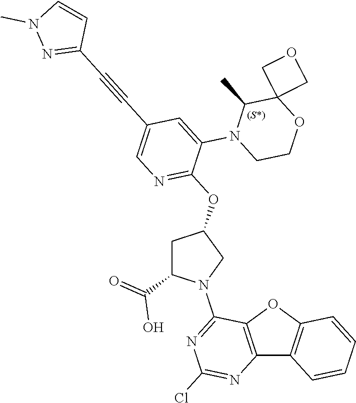

2,4-Dichlorobenzofuro[3,2-d]pyrimidine (145.0 mg, 0.6065 mmol), a stir bar, DMSO (3.0 mL), (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15, 700.0 mg crude as TFA salt) and DIEA (238.0 mg, 1.841 mmol) were added to a 40 mL vial. The resulting yellow suspension was stirred at 110° C. for 1 h, cooled to rt, triturated with H₂O (15 mL) at rt for 10 min and filtered. The filter cake was washed with H₂O (10 mL), collected and concentrated to dryness in vacuo to give a yellow solid, which was subjected to silica gel chromatography (0-100% EtOAc/pet ether) to yield (2S,4S)-1-(2-chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid as a yellow solid (150 mg, 35%). LCMS (ESI): mass calcd for C₃₃H₃₀ClN₇O₆, 655.2; m/z found, 656.2 [M+H]⁺. ¹H NMR (400 MHz, CDCl₃) δ 8.16 (d, J=8.0 Hz, 1H), 8.03 (s, 1H), 7.66-7.55 (m, 2H), 7.47-7.40 (m, 1H), 7.37 (d, J=2.0 Hz, 1H), 7.22 (s, 1H), 6.47 (d, J=2.4 Hz, 1H), 6.36-6.29 (m, 1H), 5.38 (s, 1H), 4.98 (d, J=6.4 Hz, 1H), 4.85-4.77 (m, 1H), 4.69-4.59 (m, 1H), 4.59-4.42 (m, 3H), 3.95 (s, 3H), 3.95-3.85 (m, 1H), 3.66-3.58 (m, 1H), 3.32-3.21 (m, 1H), 2.93-2.69 (m, 2H), 2.66-2.57 (m, 1H), 0.91 (d, J=6.4 Hz, 3H).

### Intermediate 17: (2S,4S)-4-((5-Bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid

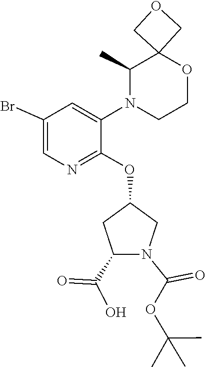 [SMILES: C[C@@H]1N(c2cc(Br)cnc2O[C@H]2C[C@@H](C(=O)O)N(C(=O)OC(C)(C)C)C2)CCOC12COC2]

Step A. (S)-8-(5-Bromo-2-nitropyridin-3-yl)-9-methyl-2,5-dioxa-8-azaspiro[3.5]nonane. A solution of 5-bromo-3-fluoro-2-nitropyridine (9.0 g, 41 mmol), (S)-9-methyl-2,5-dioxa-8-azaspiro[3.5]nonane (5.0 g, 35 mmol), and K₂CO₃ (14.0 g, 101 mmol) in MeCN (125 mL) was stirred at 80° C. overnight. The reaction mixture was cooled to rt and the resulting yellow suspension was filtered. The filter cake was rinsed with ethyl acetate (400 mL) and the resulting filtrate was concentrated to dryness in vacuo to give a yellow oil. The resulting yellow oil was purified (FCC, SiO₂, 0-40% EtOAc/pet ether) to afford the title compound, (S)-8-(5-bromo-2-nitropyridin-3-yl)-9-methyl-2,5-dioxa-8-azaspiro[3.5]nonane as a yellow solid (4 g, 32%). LCMS (ESI): mass calcd for C₁₂H₁₄BrN₃O₄, 343.0; m/z found, 344.0 [M+H]⁺. ¹H NMR (400 MHz, CDCl₃) δ=8.20 (d, J=1.6 Hz, 1H), 7.73 (d, J=1.6 Hz, 1H), 4.74 (d, J=6.8 Hz, 1H), 4.60 (d, J=6.8 Hz, 1H), 4.52 (d, J=7.2 Hz, 1H), 4.42 (d, J=7.2 Hz, 1H), 3.87-3.70 (m, 3H), 3.41-3.33 (m, 1H), 2.75-2.66 (m, 1H), 1.14 (d, J=6.4 Hz, 3H).

Step B. (2S,4S)-4-((5-Bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid. A solution of (S)-8-(5-bromo-2-nitropyridin-3-yl)-9-methyl-2,5-dioxa-8-azaspiro[3.5]nonane (3.00 g, 8.72 mmol), and (2S,4S)-1-(tert-butoxycarbonyl)-4-hydroxypyrrolidine-2-carboxylic acid (3.08 g, 13.3 mmol) in DMF (42.0 mL), was subjected to three cycles of vacuum and recharging with N₂. The resulting reaction mixture was cooled to 0° C. and NaH (60% in mineral oil, 1.61 g, 40.3 mmol) was added. The reaction mixture was removed from the ice bath and stirred at rt overnight. The reaction mixture was cooled to 0° C., and H₂O (80 mL) was slowly added via syringe over a course of 10 min. To the reaction mixture was added saturated citric acid until pH measured 6. The resulting reaction mixture was extracted with (60 mL×4) and the combined extracts were dried over anhydrous Na₂SO₄, filtered and concentrated to dryness in vacuo to give a yellow oil. The resulting yellow oil was purified (FCC, SiO₂, 0-40% ethyl acetate/petroleum ether) to afford the title compound, (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid as a yellow solid (3.7 g, 77%). LCMS (ESI): mass calcd for C₂₂H₃₀BrN₃O₇, 527.1; m/z found, 528.1 [M+H]⁺. ¹H NMR (400 MHz, DMSO-d₆) δ=12.54 (br s, 1H), 7.85 (s, 1H), 7.39-7.19 (m, 1H), 5.39-5.22 (m, 1H), 4.67-4.57 (m, 2H), 4.48-4.43 (m, 1H), 4.37-4.29 (m, 1H), 4.28-4.22 (m, 1H), 4.19-4.14 (m, 1H), 3.89-3.80 (m, 1H), 3.80-3.74 (m, 1H), 3.65-3.58 (m, 1H), 3.54-3.45 (m, 1H), 3.29-3.21 (m, 1H), 2.86-2.69 (m, 1H), 2.66-2.57 (m, 1H), 2.30-2.18 (m, 1H), 1.38 (d, J=16.8 Hz, 9H), 0.86-0.69 (m, 3H).

### Intermediate 18: 4-Chloro-2-ethylbenzofuro[3,2-d]pyrimidine

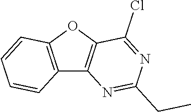 [SMILES: CCc1nc(Cl)c2oc3ccccc3c2n1]

Step A. N-(2-Cyanobenzofuran-3-yl)propionamide. To a cooled (0° C.) solution of 3-aminobenzofuran-2-carbonitrile (1.00 g, 6.32 mmol), in pyridine (8.0 mL) was added propionic anhydride (1.60 g, 12.3 mmol) dropwise over 5 min. The resulting solution was warmed to 100° C., and stirred for 2 days. The resulting reaction mixture was cooled to rt. The reaction mixture was diluted with H₂O (10 mL) and extracted with EtOAc (15 mL×3). The combined extracts were dried over anhydrous Na₂SO₄, filtered, and concentrated to dryness in vacuo to afford the title compound, N-(2-cyanobenzofuran-3-yl)propionamide (600.0 mg, 49%, crude) as a brown solid. LCMS (ESI): mass calcd for C₁₂H₁₀N₂O₂, 214.1; m/z found, 215.1 [M+H]⁺. ¹H NMR (400 MHz, DMSO-d₆) δ=10.74 (br s, 1H), 8.04 (d, J=8.0 Hz, 1H), 7.68-7.65 (m, 1H), 7.64-7.58 (m, 1H), 7.46-7.41 (m, 1H), 2.76-2.66 (m, 2H), 1.06-0.99 (m, 3H).

Step B. 4-Chloro-2-ethylbenzofuro[3,2-d]pyrimidine. A mixture of N-(2-cyanobenzofuran-3-yl)propionamide (100.0 mg, 0.3460 mmol), PCIs (288.0 mg, 1.383 mmol) and sulfolane (3.0 mL) was stirred at 110° C. for 2 h. The reaction mixture was cooled to rt, quenched with ice-water (6 mL) at 0° C., and extracted with EtOAc (10 mL×3). The combined extracts were dried over anhydrous Na₂SO₄, filtered and concentrated to dryness in vacuo to give a brown solid. The resulting solid was purified (FCC, SiO₂, 0-8% EtOAc/pet ether) to afford the title compound, 4-chloro-2-ethylbenzofuro[3,2-d]pyrimidine (30.0 mg, 37%) as a brown solid. LCMS (ESI): mass calcd for C₁₂H₉ClN₂O, 232.0; m/z found, 233.0 [M+H]⁺. ¹H NMR (400 MHz, DMSO-d₆) δ=8.27 (d, J=7.2 Hz, 1H), 7.97 (d, J=8.4 Hz, 1H), 7.91-7.82 (m, 1H), 7.66-7.57 (m, 1H), 3.11-3.02 (m, 2H), 1.37 (t, J=7.6 Hz, 3H).

### Intermediate 19: (2S,4S)-4-((5-((1,3-Dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate

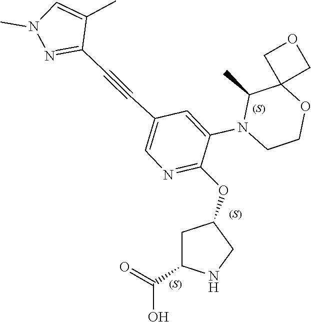

Step A. (2S,4S)-1-(tert-Butoxycarbonyl)-4-((5-((1,4-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid. A solution of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (Intermediate 17, 600.0 mg, 1.086 mmol), 3-ethynyl-1,4-dimethyl-1H-pyrazole (132.0 mg, 1.099 mmol), Cs₂CO₃ (1.07 g, 3.28 mmol), Xphos (110.0 mg, 0.2307 umol) and Xphos Pd G3 (110.0 mg, 01298 mmol) in MeCN (12.0 mL) was purged with nitrogen and heated at 70° C. for 2 h. The reaction mixture was cooled to rt and the resulting brown suspension was filtered. The resulting filter cake was rinsed with EtOAc (50 mL) and the filtrate was concentrated in vacuo to give a black solid. Purification of the resulting residue (FCC, SiO₂, 0-100% ethyl acetate/petroleum ether, then 0-15% MeOH/DCM) afforded the title compound, (2S,4S)-1-(tert-butoxycarbonyl)-4-((5-((1,4-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid as a brown solid (650.0 mg, 81%). LCMS (ESI): mass calcd for C₂₉H₃₇N₅O₇, 567.3; m/z found, 568.2 [M+H]⁺. ¹H NMR (400 MHz, DMSO-d₆) δ=7.95 (s, 1H), 7.56 (s, 1H), 7.34-7.17 (m, 1H), 5.41-5.32 (m, 1H), 4.70-4.58 (m, 2H), 4.50-4.44 (m, 1H), 4.35-4.23 (m, 2H), 4.19-4.15 (m, 1H), 4.13-4.07 (m, 1H), 3.92-3.83 (m, 1H), 3.79 (s, 3H), 3.78-3.74 (m, 1H), 3.68-3.59 (m, 1H), 3.56-3.47 (m, 1H), 2.87-2.75 (m, 1H), 2.69-2.61 (m, 1H), 2.30-2.20 (m, 1H), 2.07 (s, 3H), 1.42-1.35 (m, 9H), 0.81-0.75 (m, 3H).

Step B. (2S,4S)-4-((5-((1,4-Dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate. A solution of (2S,4S)-1-(tert-butoxycarbonyl)-4-((5-((1,4-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (200.0 mg, 0.2716 umol) in TFA/DCM (8.0 mL, 1:4) was stirred at rt for 1 h. The resulting yellow solution was concentrated to dryness in vacuo to afford the title compound, (2S,4S)-4-((5-((1,4-dimethyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate as a yellow oil (200.0 mg, 127%) which was used without further purification. LCMS (ESI): mass calcd for C₂₄H₂₉N₅O₅·C₂HF₃O₂, 467.2; m/z found, 468.1 [M+H]⁺.

Intermediates 22-24, 27 and 28 in Table 4 were prepared in a manner analogous to Intermediate 19, Steps A-B using the appropriate alkyne instead of 3-ethynyl-1,4-dimethyl-1H-pyrazole in Step A.

TABLE 4

| Int. # | Structure | alkyne | MS and ¹H-NMR |
|---|---|---|---|
| 22 | 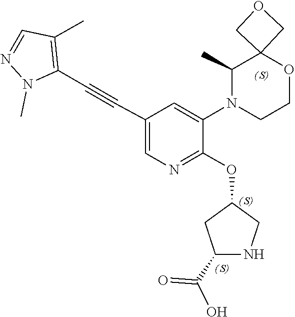 | 5-ethynyl-1,4- dimethyl- 1H-pyrazole | LCMS (ESI): mass calcd for C₂₄H₂₉N₅O₅, 467.2; m/z found, 468.2 [M + H]⁺. ¹H NMR (400 MHz, CDCl₃) δ 8.02 (d, J = 1.6 Hz, 1H), 7.56 (s, 1H), 7.19 (s, 1H), 6.02 − 5.91 (m, 1H), 5.05 − 4.99 (m, 1H), 4.95 − 4.90 (m, 1H), 4.78 − 4.71 (m, 1H), 4.68 − 4.63 (m, 1H), 4.61 − 4.53 (m, 1H), 4.42 − 4.30 (m, 1H), 4.05 (s, 3H), 3.99 − 3.92 (m, 2H), 3.91 − 3.83 (m, 1H), 3.76 − 3.64 (m, 1H), 3.45 − 3.30 (m, 1H), 2.95 − 2.88 (m, 2H), 2.72 − 2.62 (m, 1H), 2.21 (s, 3H), 0.94 (d, J = 6.4 Hz, 3H) |
| | (2S,4S)-4-((5-((1,4-Dimethyl-1H- | | |
| | pyrazol-5-yl)ethynyl)-3-((S)-9- | | |
| | methyl-2,5-dioxa-8- | | |
| | azaspiro[3.5]nonan-8-yl)pyridin- | | |
| | 2-yl)oxy)pyrrolidine-2-carboxylic | | |
| | acid trifluoroacetate | | |
| 23 | 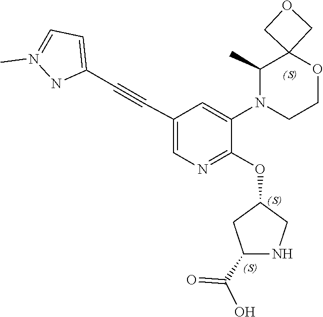 | 3-ethynyl-1-methyl-1H- pyrazole | LCMS (ESI): mass calcd for C₂₃H₂₇N₅O₅, 453.2; m/z found, 454.3 [M + H]⁺. |
| | (2S,4S)-4-((5-((1-methyl-1H- | | |
| | pyrazol-3-yl)ethynyl)-3-((S)-9- | | |
| | methyl-2,5-dioxa-8- | | |
| | azaspiro[3.5]nonan-8-yl)pyridin- | | |
| | 2-yl)oxy)pyrrolidine-2-carboxylic | | |
| | acid trifluoroacetate | | |
| 24 | 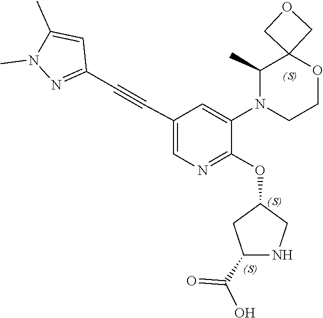 | 3-ethynyl-1,5-dimethyl- 1H-pyrazole | LCMS (ESI): mass calcd for C₂₄H₂₉N₅O₅, 467.2; m/z found, 468.1 [M + H]⁺. |
| | (2S,4S)-4-((5-((1,5-Dimethyl-1H- | | |
| | pyrazol-3-yl)ethynyl)-3-((S)-9- | | |
| | methyl-2,5-dioxa-8- | | |
| | azaspiro[3.5]nonan-8-yl)pyridin- | | |
| | 2-yl)oxy)pyrrolidine-2-carboxylic | | |
| | acid trifluoroacetate | | |
| 27 | 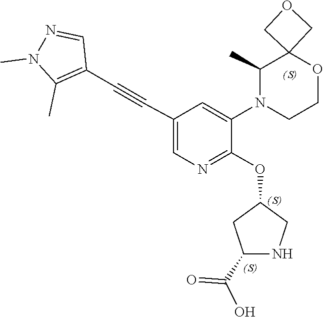 | 4-ethynyl-1,5-dimethyl- 1H-pyrazole | LCMS (ESI): mass calcd for C₂₄H₂₉N₅O₅, 467.2; m/z found, 468.1 [M + H]⁺. |
| | (2S,4S)-4-((5-((1,5-dimethyl-1H- | | |
| | pyrazol-4-yl)ethynyl)-3-((S)-9- | | |
| | methyl-2,5-dioxa-8- | | |
| | azaspiro[3.5]nonan-8-yl)pyridin- | | |
| | 2-yl)oxy)pyrrolidine-2-carboxylic | | |
| | acid trifluoroacetate | | |
| 28 | 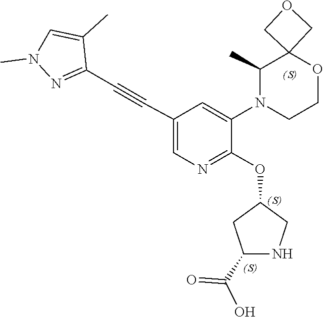 | 3-ethynyl-1,4-dimethyl- 1H-pyrazole | LCMS (ESI): mass calcd for C₂₄H₂₉N₅O₅, 467.2; m/z found, 468.2 [M + H]⁺. |
| | (2S,4S)-4-((5-((1,4-dimethyl-1H- | | |
| | pyrazol-3-yl)ethynyl)-3-((S)-9- | | |
| | methyl-2,5-dioxa-8- | | |
| | azaspiro[3.5]nonan-8-yl)pyridin- | | |
| | 2-yl)oxy)pyrrolidine-2-carboxylic | | |
| | acid trifluoroacetate | | |

### Intermediate 20: (2S,4S)-4-((5-((1,3-Dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate

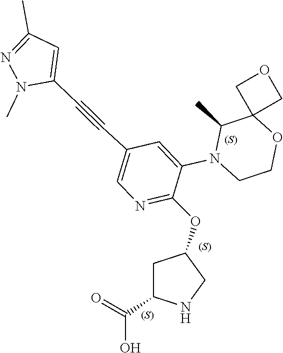

Step A. (2S,4S)-1-(tert-Butoxycarbonyl)-4-((5-((1,3-dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid. A mixture of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (Intermediate 17, 500.0 mg, 0.9384 mmol), ACN (8.0 mL), 5-ethynyl-1,3-dimethyl-1H-pyrazole (135.0 mg, 1.124 mmol), XPhos Pd G3 (82.0 mg, 0.0968 mmol), Xphos (73.0 mg, 0.153 mmol), and Cs₂CO₃ (925.0 mg, 2.839 mmol), under nitrogen, was stirred at 70° C. for 2 h. The mixture was cooled to rt, filtered through a pad of Celite®, and the filtrate concentrated to dryness in vacuo to give a yellow oil, which was subjected to HPLC (Xtimate C18, 10 μm, 150 mm×40 mm; 7 min gradient (47-67% ACN/H₂O (with 0.2% FA)) at 55 mL/min) to give (2S,4S)-1-(tert-butoxycarbonyl)-4-((5-((1,3-dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid as a brown solid (390.0 mg, 70%). LCMS (ESI): mass calcd for C₂₉H₃₇N₅O₇, 567.3; m/z found, 568.3 [M+H]⁺. ¹H NMR (400 MHz, DMSO-d₆) δ=12.48 (br s, 1H), 8.08-7.97 (m, 1H), 7.38-7.27 (m, 1H), 6.36 (s, 1H), 5.45-5.29 (m, 1H), 4.69-4.57 (m, 2H), 4.50-4.42 (m, 1H), 4.37-4.23 (m, 2H), 4.20-4.14 (m, 1H), 3.91-3.87 (m, 1H), 3.85 (s, 3H), 3.82-3.75 (m, 1H), 3.68-3.59 (m, 1H), 3.57-3.47 (m, 1H), 3.28-3.23 (m, 1H), 2.89-2.75 (m, 1H), 2.69-2.61 (m, 1H), 2.33-2.20 (m, 1H), 2.15 (s, 3H), 1.43-1.33 (m, 9H), 0.83-0.71 (m, 3H).

Step B. (2S,4S)-4-((5-((1,3-Dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate. (2S,4S)-1-(tert-Butoxycarbonyl)-4-((5-((1,3-dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid (150.0 mg, 0.2533 mmol), and TFA/DCM (3.0 mL, 1:5) was stirred at rt for 2 h, then concentrated to dryness in vacuo to give (2S,4S)-4-((5-((1,3-dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate as a brown oil (165.5 mg, 100%), which was used without further purification. LCMS (ESI): mass calcd for C₂₄H₂₉N₅O₅, 467.2; m/z found, 468.2 [M+H]⁺. ¹H NMR (400 MHz, CDCl₃) δ=8.02-7.84 (m, 1H), 7.15-6.99 (m, 1H), 6.25 (s, 1H), 5.69-5.53 (m, 1H), 4.92-4.84 (m, 1H), 4.80-4.73 (m, 1H), 4.63-4.54 (m, 1H), 4.43-4.31 (m, 2H), 4.28-4.19 (m, 1H), 3.91 (s, 3H), 3.88-3.83 (m, 1H), 3.76-3.66 (m, 3H), 3.41-3.33 (m, 1H), 2.81-2.66 (m, 2H), 2.61-2.54 (m, 1H), 2.27 (s, 3H), 0.89 (d, J=6.4 Hz, 3H).

### Intermediate 21: (2S,4S)-4-((3-((S)-9-Methyl-2,5-dioxa-8-azaspiro[3.5]nonan-8-yl)-5-((2-methylpyridin-3-yl)ethynyl)pyridin-2-yl)oxy)pyrrolidine-2-carboxylic acid

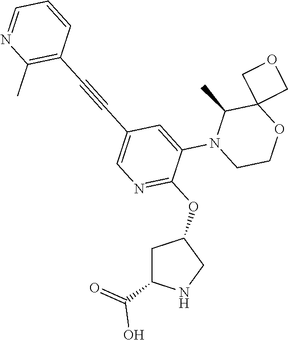 [SMILES: Cc1ncccc1C#Cc1cnc(O[C@@H]2CN[C@H](C(=O)O)C2)c(N2CCOC3(COC3)[C@@H]2C)c1]

Step A. (2S,4S)-1-(tert-Butoxycarbonyl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid. Under nitrogen, a mixture of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (500.0 mg, 0.9463 mmol), ACN (8.0 mL), 3-ethynyl-2-methylpyridine (333.0 mg, 2.843 mmol), XPhos Pd G3 (83.0 mg, 0.0979 mmol), Xphos (73.0 mg, 0.153 mmol), and Cs₂CO₃ (924.0 mg, 2.836 mmol) was stirred at 70° C. for 2 h. The reaction mixture was cooled to rt and filtered through a pad of Celite®. The resulting filtrate was concentrated to dryness in vacuo to give a yellow solid, which was purified (FCC, SiO₂, 0-100% EtOAc/pet ether, then 0-20% MeOH/DCM) followed by HPLC (Phenomenex Gemini NX C18, 5 μm, 150 mm×30 mm; 7 min gradient (33-63% ACN/H₂O (with 0.2% FA)) at 25 mL/min) to afford the title compound, (2S,4S)-1-(tert-butoxycarbonyl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid as a brown solid (200.0 mg, 35%). LCMS (ESI): mass calcd for C₃₀H₃₆N₄O₇, 564.3; m/z found, 565.2 [M+H]⁺. ¹H NMR (400 MHz, DMSO-d₆) δ=12.53 (br s, 1H), 8.47-8.42 (m, 1H), 8.02 (s, 1H), 7.88 (d, J=7.6 Hz, 1H), 7.32-7.29 (m, 1H), 7.29-7.25 (m, 1H), 5.44-5.33 (m, 1H), 4.71-4.59 (m, 2H), 4.50-4.44 (m, 1H), 4.39-4.23 (m, 2H), 4.21-4.14 (m, 1H), 3.93-3.84 (m, 1H), 3.83-3.76 (m, 1H), 3.69-3.59 (m, 1H), 3.58-3.47 (m, 1H), 3.28-3.24 (m, 1H), 2.91-2.71 (m, 1H), 2.68-2.66 (m, 1H), 2.66 (s, 3H), 2.35-2.17 (m, 1H), 1.44-1.33 (m, 9H), 0.83-0.74 (m, 3H).

Step B. (2S,4S)-4-((3-((S)-9-Methyl-2,5-dioxa-8-azaspiro[3.5]nonan-8-yl)-5-((2-methylpyridin-3-yl)ethynyl)pyridin-2-yl)oxy)pyrrolidine-2-carboxylic acid trifluoroacetate. A mixture of (2S,4S)-1-(tert-butoxycarbonyl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid (150.0 mg, 0.2504 mmol), and TFA/DCM (3.0 mL, 1:5) was stirred at rt for 2 h. The reaction mixture was concentrated to dryness in vacuo to give (2S,4S)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid trifluoroacetate as a brown oil (116.3 mg, 80%), which was used without further purification. LCMS (ESI): mass calcd for C₂₅H₂₈N₄O₅, 464.2; m/z found, 465.1 [M+H]⁺.

### Intermediate 25: (2S,4S)-4-((5-((1-Methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate

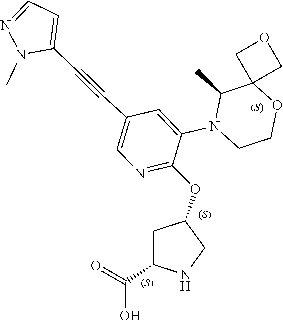

Step A. (2S,4S)-1-(tert-Butoxycarbonyl)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid. Under nitrogen, a mixture of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (Intermediate 17, 500.0 mg, 0.9384 mmol), ACN (20.0 mL), 5-ethynyl-1-methyl-1H-pyrazole (182.0 mg, 1.715 mmol), Cs₂CO₃ (818.0 mg, 2.511 mmol), Xphos (124.0 mg, 0.2601 mmol), and Xphos Pd G3 (124.0 mg, 0.1463 mmol) was stirred at 70° C. for 2 h. The resulting reaction mixture was filtered through a pad of Celite®, the filter cake was washed with EtOAc (10 mL). The filtrate was concentrated to dryness in vacuo to afford a brown solid. The resulting residue was purified, HPLC (Xtimate C18, 10 μm, 150 mm×40 mm; 7 min gradient (47-77% ACN/H₂O (with 0.2% FA)) at 55 mL/min) to afford the title compound, (2S,4S)-1-(tert-butoxycarbonyl)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid as a yellow solid (500.0 mg, 96%). LCMS (ESI): mass calcd for C₂₈H₃₅N₅O₇, 553.3; m/z found, 554.2 [M+H]⁺. ¹H NMR (400 MHz, DMSO-d₆) δ=8.07-8.01 (m, 1H), 7.51 (d, J=2.0 Hz, 1H), 7.35-7.30 (m, 1H), 6.63-6.57 (m, 1H), 5.44-5.33 (m, 1H), 4.70-4.59 (m, 2H), 4.50-4.44 (m, 1H), 4.37-4.24 (m, 2H), 4.20-4.15 (m, 1H), 3.94 (s, 3H), 3.91-3.85 (m, 1H), 3.82-3.77 (m, 1H), 3.68-3.59 (m, 1H), 3.57-3.47 (m, 1H), 3.28-3.23 (m, 1H), 2.90-2.75 (m, 1H), 2.68-2.61 (m, 1H), 2.33-2.20 (m, 1H), 1.41-1.36 (m, 9H), 0.88-0.81 (m, 3H).

Step B. (2S,4S)-4-((5-((1-Methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate. A mixture of (2S,4S)-1-(tert-Butoxycarbonyl)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid (150.0 mg, 0.2710 mmol), and TFA/DCM (5.0 mL, 1:5) was stirred at rt for 1 h, then concentrated to dryness in vacuo to afford the title compound, (2S,4S)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate as a brown oil (330.0 mg), which was used without further purification. LCMS (ESI): mass calcd for C₂₃H₂₇N₅O₅, 453.2; m/z found, 454.1 [M+H]⁺. Intermediate 26: (2S,4S)-4-((5-((1,3-Dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate

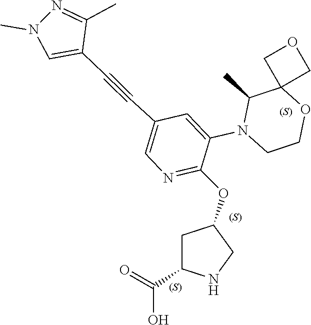

Step A. (2S,4S)-1-(tert-Butoxycarbonyl)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid. Under nitrogen, a mixture of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (Intermediate 17, 300.0 mg, 0.5430 mmol), a stir bar, ACN (6.0 mL), 4-ethynyl-1,3-dimethyl-1H-pyrazole (97.8 mg, 0.814 mmol), Xphos Pd G3 (46.0 mg, 0.0543 mmol), Xphos (31.1 mg, 0.0652 mmol), and Cs₂CO₃ (530.8 mg, 1.629 mmol) was stirred at 70° C. for 2 h. The reaction mixture was cooled to rt, passed through a syringe filter (0.45 μm nylon membrane) and subjected to HPLC (Xtimate C18, 10 μm, 150 mm×40 mm; 7 min gradient (45-75% ACN/H₂O (with 0.2% FA)) at 55 mL/min) to afford the title compound, (2S,4S)-1-(tert-butoxycarbonyl)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid as a white solid (230.0 mg, 75%). LCMS (ESI): mass calcd for C₂₉H₃₇N₅O₇, 567.3; m/z found, 568.2 [M+H]⁺. ¹H NMR (400 MHz, DMSO-d₆) δ=7.91 (s, 1H), 7.90 (s, 1H), 7.20 (s, 1H), 5.38-5.31 (m, 1H), 4.70-4.58 (m, 2H), 4.49-4.43 (m, 1H), 4.36-4.22 (m, 2H), 4.17 (d, J=6.8 Hz, 1H), 3.90-3.83 (m, 1H), 3.82-3.77 (m, 1H), 3.76 (s, 3H), 3.67-3.59 (m, 1H), 3.55-3.44 (m, 1H), 3.43-3.38 (m, 1H), 3.29-3.25 (m, 1H), 2.89-2.73 (m, 1H), 2.64-2.58 (m, 1H), 2.22 (s, 3H), 1.42-1.34 (m, 9H), 0.80-0.75 (m, 3H).

Step B. (2S,4S)-4-((5-((1,3-Dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate. A mixture of (2S,4S)-1-(tert-Butoxycarbonyl)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid (130.0 mg, 0.2290 mmol), and DCM/TFA (2.00 mL, 5:1) was stirred at rt for 1 h. The reaction mixture was concentrated to dryness in vacuo to afford the title compound, (2S,4S)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate as a brown oil (150.0 mg), which was used without further purification. LCMS (ESI): mass calcd for C₂₄H₂₉N₅O₅, 467.2; m/z found, 468.2 [M+H]⁺.

### Intermediate 29: (2S,4S)-4-((5-Bromo-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid

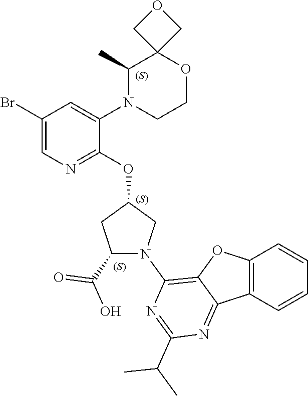

(2S,4S)-4-((5-Bromo-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid. A solution of (2S,4S)-4-hydroxy-1-(2-isopropylbenzofuro[3,2-d]pyrimidin-4-yl)pyrrolidine-2-carboxylic acid (Intermediate 30, product from Step A, 500.0 mg, 1.465 mmol), (S)-8-(5-bromo-2-nitropyridin-3-yl)-9-methyl-2,5-dioxa-8-azaspiro[3.5]nonane (Intermediate 17, product from Step A, 504.0 mg, 1.378 mmol), in DMF (10.0 mL) was subjected to three cycles of vacuum and recharging with N₂. The resulting reaction mixture was cooled to 0° C., and NaH (60% in mineral oil, 355.0 mg, 8.876 mmol) was added and subjected to three cycles of vacuum and recharging with N₂. The reaction mixture was removed from the ice bath and stirred at rt overnight. The reaction mixture was treated with aq HCl (1 M) until pH 3 was achieved at 0° C. (ice/water) to give an orange suspension. The reaction mixture was extracted with DCM/MeOH (v/v, 10:1) (15 mL×4). The combined extracts were dried over anhydrous Na₂SO₄, filtered and concentrated to dryness in vacuo. The resulting residue was purified by HPLC (Welch Xtimate C18, 5 μm, 150 mm×30 mm; 7 min isocratic (30-60% ACN (H₂O(0.05% of 30% aq NH₃+10 mM NH₄HCOs)) at 25 mL/min)) to afford the title compound, (2S,4S)-4-((5-bromo-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid as a white powder (590.0 mg, 63%). LCMS (ESI): mass calcd for C₃₀H₃₂BrN₅O₆, 637.2; m/z found, 638.1 [M+H]⁺. ¹H NMR (400 MHz, DMSO-d₆) δ=13.01-12.56 (m, 1H), 8.09 (d, J=7.6 Hz, 1H), 7.92 (s, 1H), 7.83-7.70 (m, 1H), 7.70-7.63 (m, 1H), 7.52-7.44 (m, 1H), 7.33 (s, 1H), 5.66-5.45 (m, 1H), 5.41-4.82 (m, 1H), 4.67-4.57 (m, 1H), 4.57-4.47 (m, 1H), 4.43-4.18 (m, 3H), 4.11-3.98 (m, 1H), 3.80-3.70 (m, 1H), 3.67-3.56 (m, 1H), 3.29-3.21 (m, 1H), 3.16-3.02 (m, 1H), 3.01-2.88 (m, 1H), 2.81-2.60 (m, 2H), 2.47-2.30 (m, 1H), 1.30 (d, J=5.6 Hz, 6H), 0.77 (d, J=6.0 Hz, 3H).

### Intermediate 30: (2S,4S)-4-((6-Chloro-4-((S)-9-methyl-2,5-dioxa-8-azaspiro[3.5]nonan-8-yl)pyridazin-3-yl)oxy)-1-(2-isopropylbenzofuro[3,2-d]pyrimidin-4-yl)pyrrolidine-2-carboxylic acid

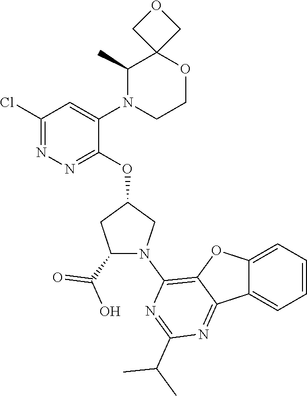 [SMILES: CC(C)c1nc(N2C[C@@H](Oc3nnc(Cl)cc3N3CCOC4(COC4)[C@@H]3C)C[C@H]2C(=O)O)c2oc3ccccc3c2n1]

Step A. (2S,4S)-4-Hydroxy-1-(2-isopropylbenzofuro[3,2-d]pyrimidin-4-yl)pyrrolidine-2-carboxylic acid. A mixture of 4-chloro-2-isopropylbenzofuro[3,2-d]pyrimidine (Intermediate 7, 1.00 g, 4.00 mmol), (2S,4S)-4-hydroxypyrrolidine-2-carboxylic acid (550.0 mg, 4.194 mmol), DIEA (2.00 mL, 11.8 mmol), and DMSO (5.0 mL) was stirred at 100° C. for 1 h. The resulting brown solution was treated with aq. HCl (1 M) until pH 3 was reached at 0° C. The reaction mixture was filtered and the filter cake was dried to afford the title compound, (2S,4S)-4-hydroxy-1-(2-isopropylbenzofuro[3,2-d]pyrimidin-4-yl)pyrrolidine-2-carboxylic acid as off-white solid (1.00 g, 56%). The filtrate was purified by reverse phase HPLC (Xtimate C18, 10 μm, 150 mm×40 mm; 2 min gradient (12-42% ACN/H₂O (with 0.02% FA)) at 25 mL/min)) to afford the title compound, (2S,4S)-4-hydroxy-1-(2-isopropylbenzofuro[3,2-d]pyrimidin-4-yl)pyrrolidine-2-carboxylic acid as a white solid (160.0 mg, 11%). LCMS (ESI): mass calcd for C₁₁H₁₃C₁₂N₃O₂, 341.1; m/z found, 342.2 [M+H]⁺. ¹H NMR (400 MHz, DMSO-d₆) δ=8.65-8.39 (m, 1H), 7.94-7.74 (m, 2H), 7.65-7.50 (m, 1H), 5.58-5.20 (m, 1H), 4.94-4.49 (m, 1H), 4.48-4.38 (m, 1H), 4.25-3.96 (m, 1H), 3.96-3.81 (m, 1H), 3.67-3.52 (m, 1H), 3.44-3.29 (m, 1H), 2.10-2.04 (m, 1H), 1.45-1.34 (m, 3H), 1.30-1.23 (m, 3H).

Step B. (S)-8-(3,6-Dichloropyidazin-4-yl)-9-methyl-2,5-dioxa-8-azaspiro[3.5]nonane. A solution of 3,4,6-trichloropyridazine (960.0 mg, 5.234 mmol), (S)-9-methyl-2,5-dioxa-8-azaspiro[3.5]nonane (600.0 mg, 4.190 mmol), DIEA (1.4 mL, 8.233 mmol) and NMP (6.0 mL), was stirred at 80° C. overnight. The reaction mixture was cooled to rt. The resulting brown solution was cooled to 0° C., and diluted with H₂O (5 mL). The resulting mixture was extracted with EtOAc (20 mL×2) and the combined extracts were washed with brine (10 mL×3), dried with anhydrous Na₂SO₄, filtered and concentrated in vacuo to dryness to give a brown oil. The brown oil was purified (FCC, SiO₂, 0-70% ethyl acetate/petroleum ether) to afford the title compound, (S)-8-(3,6-dichloropyridazin-4-yl)-9-methyl-2,5-dioxa-8-azaspiro[3.5]nonane as a yellow solid (1.0 g, 78%). LCMS (ESI): mass calcd for C₁₁H₁₃Cl₂N₃O₂, 289.0; m/z found, 290.0 [M+H]⁺.

¹H NMR (400 MHz, CDCl₃) δ=6.88 (s, 1H), 4.87-4.83 (m, 1H), 4.80-4.71 (m, 2H), 4.57-4.49 (m, 1H), 4.44-4.38 (m, 1H), 3.99-3.91 (m, 1H), 3.82-3.70 (m, 1H), 3.51-3.41 (m, 1H), 2.93-2.86 (m, 1H), 1.09 (d, J=6.8 Hz, 3H).

Step C. (2S,4S)-4-((6-Chloro-4-((S)-9-methyl-2,5-dioxa-8-azaspiro[3.5]nonan-8-yl)pyridazin-3-yl)oxy)-1-(2-isopropylbenzofuro[3,2-d]pyrimidin-4-yl)pyrrolidine-2-carboxylic acid. A cooled, 0° C., solution of (2S,4S)-4-hydroxy-1-(2-isopropylbenzofuro[3,2-d]pyrimidin-4-yl)pyrrolidine-2-carboxylic acid (Intermediate 30 product from Step A, 300.0 mg, 0.6653 mmol), in THF (2.2 mL) was subjected to three cycles of vacuum and recharging with nitrogen. To the reaction mixture was added t-BuOK (1 M in THF, 3.0 mL) dropwise via syringe over the course of 2 min to give a yellow solution. The resulting reaction mixture stirred for 0.5 h. The reaction mixture was treated with a solution of (S)-8-(3,6-dichloropyridazin-4-yl)-9-methyl-2,5-dioxa-8-azaspiro[3.5]nonane (Intermediate 30 product from Step B, 300.0 mg, 0.9834 mmol) in THF (0.80 mL) via syringe over 2 min. The reaction mixture was removed from the ice bath and stirring continued for 3 h. The reaction mixture was re-cooled to 0° C., treated with sat NH₄Cl (6 mL), followed by aq HCl (1 M) until the pH measured 5. The resulting reaction mixture was diluted with H₂O (4 mL), extracted with EtOAc/MeOH (30 mL×3, 20:1), and the combined extracts dried over anhydrous Na₂SO₄, filtered, and concentrated in vacuo to dryness to give a brown oil. The resulting oil was purified (FCC, SiO₂, 0-100% EtOAc (with 0.1% AcOH)/petroleum ether) to afford a mixture of (2S,4S)-4-((6-chloro-4-((S)-9-methyl-2,5-dioxa-8-azaspiro[3.5]nonan-8-yl)pyridazin-3-yl)oxy)-1-(2-isopropylbenzofuro[3,2-d]pyrimidin-4-yl)pyrrolidine-2-carboxylic acid and (2S,4S)-4-((6-chloro-5-((S)-9-methyl-2,5-dioxa-8-azaspiro[3.5]nonan-8-yl)pyridazin-3-yl)oxy)-1-(2-isopropylbenzofuro[3,2-d]pyrimidin-4-yl)pyrrolidine-2-carboxylic acid as a yellow solid (350 mg, 44%). LCMS (ESI): mass calcd for C₂₉H₃₁ClN₆O₆, 594.2; m/z found, 595.3 [M+H]⁺. The mixture was purified (HPLC (Welch Xtimate C18, 5 μm, 150 mm×30 mm; 7 min gradient (28-58% ACN/H₂O (with 0.02% FA)) at 25 mL/min)) afforded the title compound, (2S,4S)-4-((6-chloro-4-((S)-9-methyl-2,5-dioxa-8-azaspiro[3.5]nonan-8-yl)pyridazin-3-yl)oxy)-1-(2-isopropylbenzofuro[3,2-d]pyrimidin-4-yl)pyrrolidine-2-carboxylic acid as a white powder (80 mg, 37% yield). LCMS (ESI): mass calcd for C₂₉H₃₁ClN₈₀, 594.2; m/z found, 595.3 [M+H]⁺. ¹H NMR (400 MHz, CDCl₃)=8.29-8.18 (m, 1H), 7.64-7.56 (m, 2H), 7.47-7.39 (m, 1H), 6.61 (s, 1H), 6.14-6.08 (m, 1H), 5.33-5.07 (m, 1H), 4.94-4.72 (m, 2H), 4.72-4.64 (m, 1H), 4.64-4.54 (m, 2H), 4.53-4.45 (m, 1H), 4.43-4.34 (m, 1H), 3.97-3.79 (m, 1H), 3.68-3.52 (m, 1H), 3.37-3.13 (m, 4H), 3.08-2.93 (m, 1H), 2.77-2.57 (m, 1H), 1.41-1.33 (m, 6H), 1.11 (d, J=6.8 Hz, 3H).

### Intermediate 31: 4-Chloro-2-(difluoromethyl)-7-fluoro-6-methylbenzofuro[3,2-d]pyrimidine

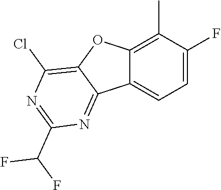 [SMILES: Cc1c(F)ccc2c1oc1c(Cl)nc(C(F)F)nc12]

Step A. 4-Fluoro-2-hydroxy-3-methylbenzonitrile. To mixture of 6-bromo-3-fluoro-2-methylphenol (1 g, 4.877 mmol) and KI (1.05 g, 6.325 mmol) in DMF (10 ml) was added CuI (1.12 g, 5.881 mmol) and CuCN (874 mg, 9.758 mmol). The reaction mixture was stirred at 120° C. for 2 d. The reaction was cooled to rt., diluted with ethyl acetate (20 mL), and washed with aq. UCI (20 mL×2, 3%). The organic layer was dried over anhydrous Na₂SO₄, filtered and concentrated to dryness in vacuo to give a yellow oil. The resulting residue was purified (FCC, SiO₂, 0-10% ethyl acetate/petroleum ether) to afford the title compound, 4-fluoro-2-hydroxy-3-methylbenzonitrile (275 mg, 33% yield) as a white solid. LCMS (ESI): mass calcd for C₈H₆FNO, 151.0; m/z found, 150.1 [M−H]⁺. ¹H NMR (400 MHz, CDCl₃) δ=7.35 (dd, J=6.1, 8.7 Hz, 1H), 6.74 (t, J=8.7 Hz, 1H), 5.92 (s, 1H), 2.20 (d, J=1.8 Hz, 3H).

Step B. 3-Amino-6-fluoro-7-methylbenzofuran-2-carbonitrile. To a mixture of 4-fluoro-2-hydroxy-3-methylbenzonitrile (270 mg, 1.556 mmol) and DMF (5 mL) under N₂, was added 2-chloroacetonitrile (162 mg, 2.146 mmol) and K₂CO₃ (756 mg, 5.470 mmol). The reaction mixture was stirred at 100° C. overnight. The reaction mixture was cooled to r.t., filtered and rinsed with ethyl acetate (20 mL). The resulting filtrate was concentrated to dryness in vacuo to give a black solid. The black solid was purified (FCC, SiO₂, 0-30% ethyl acetate/petroleum ether) to afford the title compound 3-amino-6-fluoro-7-methylbenzofuran-2-carbonitrile (250 mg, 83% yield) as yellow solid. LCMS (ESI): mass calcd for C₁₀H₇FN₂O, 190.1; m/z found, 191.0 [M+H]⁺. ¹H NMR (400 MHz, CDCl₃) δ=7.30 (dd, J=5.0, 8.7 Hz, 1H), 7.04 (t, J=9.2 Hz, 1H), 4.24 (br s, 2H), 2.38 (d, J=1.5 Hz, 3H).

Step C. N-(2-Cyano-6-fluoro-7-methylbenzofuran-3-yl)-2,2-difluoroacetamide. A mixture of 2,2-difluoroacetic anhydride (700 mg, 4.022 mmol), and a solution 3-amino-6-fluoro-7-methylbenzofuran-2-carbonitrile (250 mg, 1.292 mmol) in pyridine (2 mL), was stirred at r.t. overnight. The reaction mixture was diluted with H₂O (10 mL), extracted with ethyl acetate (15 mL×2). The combined extracts were dried over anhydrous Na₂SO₄, filtered and concentrated to dryness in vacuo. The resulting residue was purified (FCC, SiO₂, 0-25% ethyl acetate/petroleum ether) to afford the title compound N-(2-cyano-6-fluoro-7-methylbenzofuran-3-yl)-2,2-difluoroacetamide (300 mg, 1.074 mmol) as yellow solid. LCMS (ESI): mass calcd for C₁₂H₇F₃N₂O₂, 268.0; m/z found, 269.1 [M+H]⁺. ¹H NMR (400 MHz, CDCl₃) δ=8.20 (br d, J=1.9 Hz, 1H), 7.56 (dd, J=4.8, 8.6 Hz, 1H), 7.15 (t, J=9.2 Hz, 1H), 6.18 (t, J=53.8 Hz, 1H), 2.45 (d, J=1.7 Hz, 3H).

Step D. 2-(Difluoromethyl)-7-fluoro-6-methylbenzofuro[3,2-d]pyrimidin-4-ol. N-(2-cyano-6-fluoro-7-methylbenzofuran-3-yl)-2,2-difluoroacetamide (200.0 mg, 0.7160 mmol), ethanol (1.50 mL), H₂O (0.30 mL), 30% hydrogen peroxide solution (440.0 mg, 3.881 mmol) and NaOH (50.0 mg, 1.25 mmol) were combined at 0° C. The reaction mixture was subjected to vacuum and recharging with N₂ three times and stirred at 80° C. for 3 h. The reaction mixture was diluted with H₂O (10 mL) and extracted with EtOAc (15 mL×2). The combined extracts were dried over anhydrous Na₂SO₄, filtered, and concentrated to dryness in vacuo. The resulting residue was purified (FCC, SiO₂, 0-100% EtOAc/pet ether) to afford the title compound 2-(difluoromethyl)-7-fluoro-6-methylbenzofuro[3,2-d]pyrimidin-4-ol (80.0 mg, 33% yield) as white solid. LCMS (ESI): mass calcd for C₁₂H₇ClF₃N₂O₂, 268.0; m/z found, 268.9 [M+H]⁺. ¹H NMR (400 MHz, DMSO-d₆) δ 13.94-13.70 (m, 1H), 7.94 (dd, J=5.2, 8.6 Hz, 1H), 7.42-7.35 (m, 1H), 7.07-6.78 (m, 1H), 2.47 (d, J=1.1 Hz, 3H).

Step E. 4-Chloro-2-(difluoromethyl)-7-fluoro-6-methylbenzofuro[3,2-d]pyrimidine. A mixture of 2-(difluoromethyl)-7-fluoro-6-methylbenzofuro[3,2-d]pyrimidin-4-ol (75.0 mg, 0.220 mmol), 1,4-dioxane (1.50 mL), DIEA (57.0 mg, 0.441 mmol) and POCl₃ (135.0 mg, 0.8800 mmol) were combined and stirred at 100° C. for 3 h. The reaction mixture was diluted with H₂O (10 mL), extracted with EtOAc (10 mL×2). The combined extracts were dried over anhydrous Na₂SO₄, filtered, and concentrated to dryness in vacuo. The resulting residue was purified (FCC, SiO₂, 0-15% EtOAc/pet ether) to afford the title compound 4-chloro-2-(difluoromethyl)-7-fluoro-6-methylbenzofuro[3,2-d]pyrimidine as a white solid (50.0 mg, 79%). LCMS (ESI): mass calcd for C₁₂H₆ClF₃N₂O, 286.0; m/z found, 286.9 [M+H]⁺. ¹H NMR (400 MHz, CDCl₃) δ=8.15-8.10 (m, 1H), 7.34-7.28 (m, 1H), 6.80 (t, J=54.8 Hz, 1H), 2.59 (d, J=1.6 Hz, 3H).

### Intermediate 32: (2S,4S)-1-(2-(Difluoromethyl)-7-fluoro-6-methylbenzofuro[3,2-d]pyrimidin-4-yl)-4-hydroxypyrrolidine-2-carboxylic acid

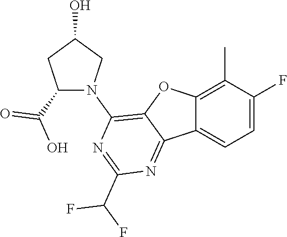 [SMILES: Cc1c(F)ccc2c1oc1c(N3C[C@@H](O)C[C@H]3C(=O)O)nc(C(F)F)nc12]

The title compound was prepared in a manner analogous to (2S,4S)-1-(2-cyclopropylbenzofuro[3,2-d]pyrimidin-4-yl)-4-hydroxypyrrolidine-2-carboxylic acid (Intermediate 38), using 4-chloro-2-(difluoromethyl)-7-fluoro-6-methylbenzofuro[3,2-d]pyrimidine (Intermediate 31) and (2S,4S)-4-hydroxypyrrolidine-2-carboxylic acid, heating at 85° C. for 0.5 h instead of 110° C. for 2 h. LCMS (ESI): mass calcd for C₁₇H₁₄FN₃O₄, 381.1; m/z found, 382.0 [M+H]⁺. ¹H NMR (400 MHz, DMSO-d₆) δ 13.02-11.74 (m, 1H), 7.97 (dd, J=5.2, 8.4 Hz, 1H), 7.33 (t, J=9.2 Hz, 1H), 6.74 (br t, J=54.8 Hz, 1H), 5.26-4.84 (m, 1H), 4.48 (br s, 1H), 4.28-3.97 (m, 1H), 3.84 (br dd, J=4.4, 7.6 Hz, 1H), 2.65-2.53 (m, 1H), 2.47 (s, 3H), 2.23 (br s, 1H), 1.19 (t, J=7.2 Hz, 1H).

### Intermediate 33. (2S,4S)-4-((6-Chloro-4-((S)-9-methyl-2,5-dioxa-8-azaspiro[3.5]nonan-8-yl)pyridazin-3-yl)oxy)-1-(2-ethylbenzofuro[3,2-d]pyrimidin-4-yl)pyrrolidine-2-carboxylic acid

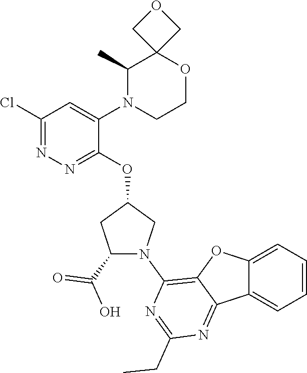 [SMILES: CCc1nc(N2C[C@@H](Oc3nnc(Cl)cc3N3CCOC4(COC4)[C@@H]3C)C[C@H]2C(=O)O)c2oc3ccccc3c2n1]

The title compound was prepared in a manner analogous to Intermediate 30 Step C using (S)-8-(3,6-dichloropyridazin-4-yl)-9-methyl-2,5-dioxa-8-azaspiro[3.5]nonane (Intermediate 30 product from Step B) and (2S,4S)-1-(2-ethylbenzofuro[3,2-d]pyrimidin-4-yl)-4-hydroxypyrrolidine-2-carboxylic acid (Intermediate 61). LCMS (ESI): mass calcd for C₂₈H₂₉ClN₆O₆ 580.2 m/z found, 581.4 [M+H]⁺. ¹H NMR (500 MHz, DMSO-d₆) δ 8.08 (dd, J=7.8, 1.0 Hz, 1H), 7.74-7.62 (m, 2H), 7.47 (t, J=7.4 Hz, 1H), 6.96 (s, 1H), 5.79 (t, J=5.8 Hz, 1H), 5.18 (s, 1H), 4.64 (q, J=6.6 Hz, 1H), 4.53 (d, J=6.8 Hz, 2H), 4.47 (d, J=6.7 Hz, 1H), 4.36 (d, J=6.9 Hz, 1H), 4.28 (s, 1H), 4.14 (d, J=7.0 Hz, 1H), 3.78 (ddd, J=11.9, 3.8, 1.4 Hz, 1H), 3.64 (td, J=11.9, 3.3 Hz, 1H), 3.34 (dd, J=13.2, 3.1 Hz, 1H), 3.25 (td, J=12.5, 3.8 Hz, 1H), 2.96 (td, J=10.1, 9.7, 5.1 Hz, 1H), 2.83 (q, J=7.6 Hz, 2H), 2.60 (d, J=14.6 Hz, 1H), 1.32 (t, J=7.6 Hz, 3H), 1.00 (d, J=6.5 Hz, 3H).

### Intermediate 34: (2S,4S)-4-((5-Ethynyl-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid

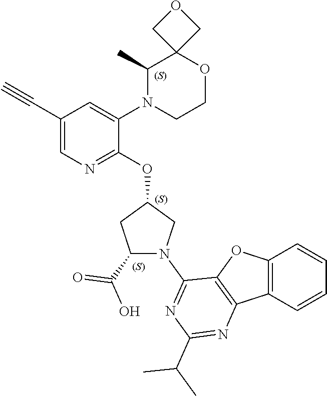

Step A. (S)-9-Methyl-8-(2-nitro-5-((triisopropylsilyl)ethynyl)pyridin-3-yl)-2,5-dioxa-8-azaspiro[3.5]nonane. In a nitrogen-purged vial, a mixture of (S)-8-(5-bromo-2-nitropyridin-3-yl)-9-methyl-2,5-dioxa-8-azaspiro[3.5]nonane (Intermediate 17, product of Step A, 200.0 mg, 0.5644 mmol), ethynyltriisopropylsilane (130.0 mg, 0.7128 mmol), DIEA (0.76 mL, 4.5 mmol), Pd(PPh₃)₂Cl₂ (20.0 mg, 0.0285 mmol), and CuI (18.0 mg, 0.0945 mmol) and ACN (10.0 mL), was stirred at 80° C. for 1 h then cooled to rt. The resulting brown suspension was treated with H₂O (20 mL), extracted with EtOAc (20 mL×3), and the combined extracts dried over anhydrous Na₂SO₄, filtered, and concentrated to dryness in vacuo to give a brown oil. The resulting oil was purified (FCC, SiO₂, 0-15% EtOAc/pet ether) to afford the title compound, (S)-9-methyl-8-(2-nitro-5-((triisopropylsilyl)ethynyl)pyridin-3-yl)-2,5-dioxa-8-azaspiro[3.5]nonane as a yellow solid (240.0 mg, 95%). LCMS (ESI): mass calcd for C₂₃H₃₅N₃O₄Si, 445.2; m/z found, 446.2 [M+H]⁺. ¹H NMR (400 MHz, CDCl₃) δ 8.19 (d, J=1.6 Hz, 1H), 7.59 (d, J=1.6 Hz, 1H), 4.74-4.70 (m, 1H), 4.62-4.58 (m, 1H), 4.53-4.48 (m, 1H), 4.45-4.40 (m, 1H), 3.85-3.77 (m, 1H), 3.76-3.66 (m, 2H), 3.38-3.30 (m, 1H), 2.74-2.68 (m, 1H), 1.18-1.09 (m, 24H).

Step B. (2S,4S)-4-((5-Ethynyl-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid. A mixture of (S)-9-Methyl-8-(2-nitro-5-((triisopropylsilyl)ethynyl)pyridin-3-yl)-2,5-dioxa-8-azaspiro[3.5]nonane (230.0 mg, 0.5161 mmol), (2S,4S)-4-hydroxy-1-(2-isopropylbenzofuro[3,2-d]pyrimidin-4-yl)pyrrolidine-2-carboxylic acid (Intermediate 30, product from Step A, 230.0 mg, 0.5101 mmol) and DMF (5.0 mL), was subjected to three cycles of vacuum and recharging with nitrogen and cooled to 0° C. (ice/water). To the cooled reaction mixture was added NaH (115.0 mg, 2.875 mmol, 60% in mineral oil) and the reaction mixture removed from the ice bath and stirred for 1 h. The reaction mixture was treated with H₂O (5 mL), passed through a syringe filter (0.22 μm nylon membrane), and concentrated to dryness in vacuo to afford a brown oil. The resulting oil was purified by reverse phase HPLC (Boston Green ODS, 5 μm, 150 mm×30 mm; 6 min gradient (27-57% ACN/H₂O (with 0.2% FA)) at 30 mL/min)) to afford the title compound, (2S,4S)-4-((5-ethynyl-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid as a yellow solid (110.0 mg, 34%). LCMS (ESI): mass calcd for C₃₂H₃₃N₅O₆, 583.2; m/z found, 584.4 [M+H]⁺. ¹H NMR (400 MHz, DMSO-d₆) δ=8.09 (d, J=8.0 Hz, 1H), 7.97 (s, 1H), 7.84-7.69 (m, 1H), 7.66 (s, 1H), 7.52-7.42 (m, 1H), 7.22 (s, 1H), 5.74-5.47 (m, 1H), 4.67-4.58 (m, 1H), 4.52 (d, J=1.6 Hz, 1H), 4.42-4.28 (m, 2H), 4.26 (s, 1H), 4.25-4.13 (m, 1H), 4.11-4.00 (m, 1H), 3.80-3.70 (m, 1H), 3.66-3.55 (m, 1H), 3.30-3.18 (m, 2H), 3.15-3.01 (m, 1H), 3.00-2.89 (m, 1H), 2.72-2.54 (m, 2H), 2.45-2.35 (m, 1H), 1.29 (s, 6H), 0.79-0.67 (m, 3H).

### Intermediate 35: (2S,4S)-1-(2-(Difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid

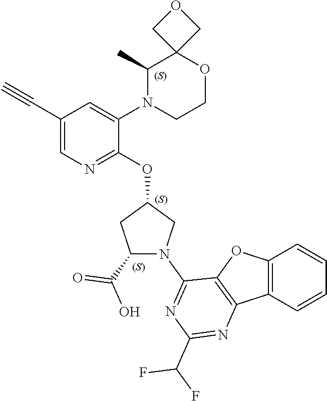

Step A. (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-((trimethylsilyl)ethynyl)pyridin-2-yl)oxy)pyrrolidine-2-carboxylic acid. A mixture of (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12, 500.0 mg, 761.7 μmol), ethynyltrimethylsilane (228.7 mg, 2.328 mmol), CuI (43.9 mg, 0.231 mmol), Pd(PPh₃)₂Cl₂ (219.4 mg, 312.6 μmol), TEA (1.1 mL, 7.7 mmol) and THF (5.0 mL) was combined under N₂. The reaction mixture was stirred at 80° C. for 1 h under N₂ to give a black suspension. Additional ethynyltrimethylsilane (149.6 mg, 1.523 mmol) and Pd(PPh₃)₂Cl₂ (219.4 mg, 312.6 μmol) were added to the reaction mixture under N₂. The reaction mixture was stirred at 80° C. for 16 h under N₂. The reaction mixture was cooled, diluted with water (10 mL) and extracted with EtOAc (20 mL×3). The combined organic layers were washed with brine (50 mL), dried over anhydrous Na₂SO₄, filtered and concentrated under reduced pressure to afford a yellow oil. The resulting oil was purified (HPLC:Phenomenex Gemini NX C18, 5 μm, 150 mm×30 mm; 7 min gradient (32-62% ACN (water(0.05% of 30% aq NH3+10 mM NH₄HCO₃)) at 25 mL/min)) to afford a mixture of (2S,4S)-1-(2-(difluoromethyl)benzofuro[3,2-d]pyrimidin-4-yl)-4-((3-((S)-9-methyl-2,5-dioxa-8-azaspiro[3.5]nonan-8-yl)-5-((trimethylsilyl)ethynyl)pyridin-2-yl)oxy)pyrrolidine-2-carboxylic acid and (2S,4S)-1-(2-(difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid (55 mg) as a yellow solid. LCMS (ESI): mass calcd for C₃₃H₃₅F₂N₅O₆Si, 663.2; m/z found, 664.3 [M+H]⁺.

Step B. (2S,4S)-1-(2-(Difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid. The mixture of (2S,4S)-1-(2-(difluoromethyl)benzofuro[3,2-d]pyrimidin-4-yl)-4-((3-((S)-9-methyl-2,5-dioxa-8-azaspiro[3.5]nonan-8-yl)-5-((trimethylsilyl)ethynyl)pyridin-2-yl)oxy)pyrrolidine-2-carboxylic acid and (2S,4S)-1-(2-(difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid from Step A (55.0 mg), K₂CO₃ (55.0 mg, 0.392 mmol) and MeOH (1.0 mL) was stirred at rt for 1 h. The reaction mixture was filtered, and the filtrate was concentrated to dryness in vacuo to afford the title compound, (2S,4S)-1-(2-(difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid (50.0 mg, 92%) as a yellow solid, which was used without further purification. LCMS (ESI): mass calcd for C₃₀H₂₇F₂N₅O₆, 591.2; m/z found, 592.2 [M+H]⁺.

### Intermediate 36: (2S,4S)-4-((5-Bromo-3-((S)-9-methyl-2,5-dioxa-8-azaspiro[3.5]nonan-8-yl)pyridin-2-yl)oxy)-1-(2-(difluoromethyl)-7-fluoro-6-methylbenzofuro[3,2-d]pyrimidin-4-yl)pyrrolidine-2-carboxylic acid

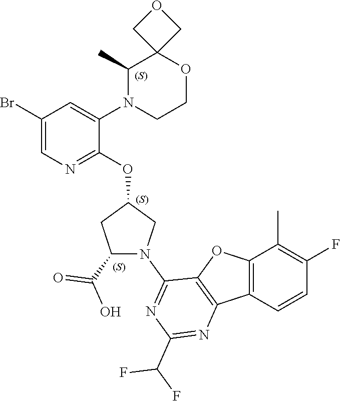

(2S,4S)-4-((5-Bromo-3-((S)-9-methyl-2,5-dioxa-8-azaspiro[3.5]nonan-8-yl)pyridin-2-yl)oxy)-1-(2-(difluoromethyl)-7-fluoro-6-methylbenzofuro[3,2-d]pyrimidin-4-yl)pyrrolidine-2-carboxylic acid. A solution of (S)-8-(5-bromo-2-nitropyridin-3-yl)-9-methyl-2,5-dioxa-8-azaspiro[3.5]nonane (Intermediate 17, product from Step A, 200.0 mg, 0.5445 mmol), and (2S,4S)-1-(2-(difluoromethyl)-7-fluoro-6-methylbenzofuro[3,2-d]pyrimidin-4-yl)-4-hydroxypyrrolidine-2-carboxylic acid (Intermediate 32, 210.0 mg, 0.5507 mmol) in DMF (5.0 mL) was subjected to three cycles of vacuum and recharging with nitrogen and cooled to 0° C. (ice/water). NaH (110.0 mg, 2.750 mmol, 60% in mineral oil) was added, the resulting brown heterogeneous mixture was removed from the ice bath and stirred at rt for 1 h. The reaction mixture was cooled to 0° C., ice water (4 mL) was added, and the reaction mixture was treated with saturated citric acid until the pH measured 6. The resulting mixture was extracted with EtOAc (4 mL×3) and the combined extracts were dried over anhydrous Na₂SO₄, filtered and concentrated to dryness in vacuo to give a brown oil. The resulting residue was purified (FCC, SiO₂, 0-40% EtOAc/pet ether) to afford the title compound, (2S,4S)-4-((5-bromo-3-((S)-9-methyl-2,5-dioxa-8-azaspiro[3.5]nonan-8-yl)pyridin-2-yl)oxy)-1-(2-(difluoromethyl)-7-fluoro-6-methylbenzofuro[3,2-d]pyrimidin-4-yl)pyrrolidine-2-carboxylic acid as a yellow solid (370.0 mg, 99%). LCMS (ESI): mass calcd for C₂₉H₂₇BrF₃N₅O₆, 677.1; m/z found, 678.3 [M+H]⁺. ¹H NMR (400 MHz, CDCl₃) δ=8.02-7.97 (m, 1H), 7.87 (d, J=2.0 Hz, 1H), 7.21-7.12 (m, 2H), 6.61 (t, J=54.8 Hz, 1H), 6.21-6.15 (m, 1H), 5.67-5.34 (m, 1H), 5.02-4.91 (m, 1H), 4.82-4.74 (m, 1H), 4.71-4.24 (m, 5H), 3.92-3.85 (m, 1H), 3.65-3.56 (m, 1H), 3.29-3.19 (m, 1H), 2.89-2.68 (m, 2H), 2.64-2.59 (m, 1H), 2.47 (s, 3H), 0.93 (d, J=6.8 Hz, 3H);

### Intermediate 37: (S)-9-Methyl-8-(2-nitro-5-(pyridine-3-ylethynyl)pyridine-3-yl)-2,5-dioxa-8-azaspiro[3,5]nonane

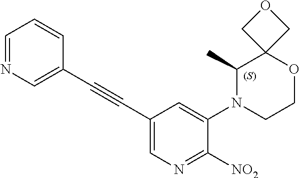

A nitrogen purged solution of (S)-8-(5-bromo-2-nitropyridin-3-yl)-9-methyl-2,5-dioxa-8-azaspiro[3.5]nonane (Intermediate 17, product from Step A, 80.0 mg, 0.232 mmol), 3-ethynylpyridine (50.0 mg, 0.485 mmol), Cs₂CO₃ (188.0 mg, 0.5770 mmol), Xphos Pd G3 (20.0 mg, 0.0236 mmol) and Xphos (12.0 mg, 0.0252 mmol) in ACN (2.00 mL), was stirred at 70° C. for 2.0 h. The reaction mixture was cooled to rt and the brown heterogeneous mixture was passed through a syringe filter (0.45 μm nylon membrane), and purified (FCC, SiO₂, 0-50% EtOAc/pet ether) to afford the title compound, (S)-9-methyl-8-(2-nitro-5-(pyridin-3-ylethynyl)pyridin-3-yl)-2,5-dioxa-8-azaspiro[3.5]nonane as a yellow powder (50.0 mg, 57%). LCMS (ESI): mass calcd for C₁₉H₁₈N₄O₄, 366.1; m/z found, 367.1 [M+H]⁺. ¹H NMR (400 MHz, CDCl₃) δ=8.83 (d, J=1.6 Hz, 1H), 8.70-8.60 (m, 1H), 8.29 (d, J=2.0 Hz, 1H), 7.92-7.80 (m, 1H), 7.73 (d, J=1.6 Hz, 1H), 7.43-7.33 (m, 1H), 4.79-4.71 (m, 1H), 4.63-4.58 (m, 1H), 4.54-4.50 (m, 1H), 4.45-4.40 (m, 1H), 3.86-3.79 (m, 1H), 3.79-3.69 (m, 2H), 3.43-3.32 (m, 1H), 2.77-2.69 (m, 1H), 1.15 (d, J=6.4 Hz, 3H).

### Intermediate 38: (2S,4S)-1-(2-Cyclopropylbenzofuro[3,2-d]pyrimidin-4-yl)-4-hydroxypyrrolidine-2-carboxylic acid

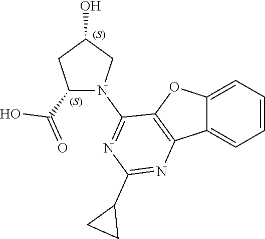

A solution of 4-chloro-2-cyclopropylbenzofuro[3,2-d]pyrimidine (Intermediate 6, 700.0 mg, 2.813 mmol), (2S,4S)-4-hydroxypyrrolidine-2-carboxylic acid (380.0 mg, 2.898 mmol) and DIEA (1.00 mL, 5.88 mmol) in DMSO (12.0 mL) was stirred at 110° C. for 2 h. The reaction mixture was cooled to rt to give a brown solution. The brown solution was directly purified by reverse phase HPLC(Phenomenex Genimi NX C18, 5 μm, 150 mm×40 mm; 8 min gradient (8-38% ACN/H₂O (with 0.2% FA)) at 55 mL/min)) to afford the title compound, (2S,4S)-1-(2-cyclopropylbenzofuro[3,2-d]pyrimidin-4-yl)-4-hydroxypyrrolidine-2-carboxylic acid formate as yellow solid (850.0 mg, 89%). LCMS (ESI): mass calcd for C18H₁₇N₃O₄, 339.1; m/z found, 340.1 [M+H]⁺. ¹H NMR (400 MHz, DMSO-d₆) δ=12.55 (br s, 1H), 8.06 (d, J=6.8 Hz, 1H), 7.84-7.60 (m, 2H), 7.54-7.38 (m, 1H), 5.47-4.83 (m, 1H), 4.67-4.44 (m, 1H), 4.40-4.28 (m, 1H), 3.94-3.73 (m, 1H), 2.49-2.42 (m, 1H), 2.29-2.10 (m, 1H), 2.08-1.84 (m, 1H), 1.09-0.81 (m, 4H).

### Intermediate 39: (2S,4S)-4-((3-((S)-9-Methyl-2,5-dioxa-8-azaspiro[3.5]nonan-8-yl)-5-(pyridin-3-ylethynyl)pyridin-2-yl)oxy)pyrrolidine-2-carboxylic acid trifluoroacetate

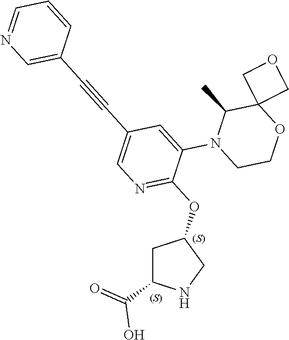

Step A. (2S,4S)-4-((5-Bromo-3-(9-methyl-2,5-dioxa-8-azaspiro[3.5]nonan-8-yl)pyridin-2-yl)oxy)-1-(tert-butoxycarbonyl)pyrrolidine-2-carboxylic acid. A solution of (S)-8-(5-bromo-2-nitropyridin-3-yl)-9-methyl-2,5-dioxa-8-azaspiro[3.5]nonane (Intermediate 17, product from Step A, 3.00 g, 8.72 mmol), and (2S,4S)-1-(tert-butoxycarbonyl)-4-hydroxypyrrolidine-2-carboxylic acid (3.08 g, 13.3 mmol) in DMF (42.0 mL), was subsequently subjected to three cycles of vacuum and recharging with N₂. To the reaction mixture was added NaH (60% in mineral oil, 1.61 g, 40.3 mmol) at 0° C. (ice/water) followed by subjection to three cycles of vacuum and recharging with N₂. The reaction mixture was removed from the ice bath and stirred at rt overnight. The reaction mixture was cooled to 0° C., treated with H₂O (80 mL) by slowly adding via syringe over a course of 10 min Saturated citric acid was added until the pH measured 6, and the reaction mixture was extracted with (60 mL×4). The combined extracts were dried over anhydrous Na₂SO₄, filtered and concentrated to dryness in vacuo to give a yellow oil. The yellow oil was purified (FCC, SiO₂, 0-40% ethyl acetate/petroleum ether) to afford the title compound, (2S,4S)-4-((5-bromo-3-(9-methyl-2,5-dioxa-8-azaspiro[3.5]nonan-8-yl)pyridin-2-yl)oxy)-1-(tert-butoxycarbonyl)pyrrolidine-2-carboxylic acid as a yellow solid (3.7 g, 77%). LCMS (ESI): mass calcd for C₂₂H₃₀BrN₃O₇, 527.1; m/z found, 528.1 [M+H]⁺. ¹H NMR (400 MHz, DMSO-d₆) δ 12.54 (br s, 1H), 7.85 (s, 1H), 7.39-7.19 (m, 1H), 5.39-5.22 (m, 1H), 4.67-4.57 (m, 2H), 4.48-4.43 (m, 1H), 4.37-4.29 (m, 1H), 4.28-4.22 (m, 1H), 4.19-4.14 (m, 1H), 3.89-3.80 (m, 1H), 3.80-3.74 (m, 1H), 3.65-3.58 (m, 1H), 3.54-3.45 (m, 1H), 3.29-3.21 (m, 1H), 2.86-2.69 (m, 1H), 2.66-2.57 (m, 1H), 2.30-2.18 (m, 1H), 1.38 (d, J=16.8 Hz, 9H), 0.86-0.69 (m, 3H).

Step B. (2S,4S)-1-(tert-Butoxycarbonyl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid. A solution of (2S,4S)-4-((5-bromo-3-((S)-9-methyl-2,5-dioxa-8-azaspiro[3.5]nonan-8-yl)pyridin-2-yl)oxy)-1-(tert-butoxycarbonyl)pyrrolidine-2-carboxylic acid (500.0 mg, 0.9050 mmol), 3-ethynylpyridine (215.0 mg, 2.085 mmol), XPhos Pd G3 (89.0 mg, 0.105 mmol), Xphos (53.0 mg, 0.111 mmol), Cs₂CO₃ (896.0 mg, 2.750 mmol), in MeCN (8.0 mL) was stirred under nitrogen at 70° C. for 2 h. The reaction mixture was cooled to rt and the resulting brown suspension was filtered through a pad of Celite®. The filtrate was concentrated to dryness in vacuo to give a yellow oil which was purified (HPLC (Xtimate C18 10 μm, 150 mm×40 mm; 7 min gradient (36-66% ACN/H₂O (with 0.2% FA)) at 55 mL/min)) to afford the title compound, (2S,4S)-1-(tert-butoxycarbonyl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid as an off-white solid (350.0 mg, 69%). LCMS (ESI): mass calcd for C₂₉H₃₄N₄O₇, 550.2; m/z found, 551.2 [M+H]⁺. ¹H NMR (400 MHz, CDCl₃) δ 8.83-8.71 (m, 1H), 8.63-8.50 (m, 1H), 7.99 (d, J=2.0 Hz, 1H), 7.83-7.79 (m, 1H), 7.32-7.28 (m, 1H), 7.18-7.12 (m, 1H), 6.12-5.70 (m, 1H), 5.01-4.74 (m, 2H), 4.72-4.35 (m, 3H), 4.32-3.99 (m, 1H), 3.93-3.80 (m, 1H), 3.77-3.61 (m, 2H), 3.60-2.93 (m, 2H), 2.70-2.57 (m, 1H), 2.56-2.42 (m, 1H), 1.94-1.83 (m, 1H), 1.51 (d, J=17.6 Hz, 9H), 0.91 (d, J=6.0 Hz, 3H).

Step C. (2S,4S)-1-(tert-Butoxycarbonyl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid trifluoroacetate (350.0 mg, 0.6276 mmol), in TFA/DCM (v/v, 1/5, 6.0 mL) was stirred at rt for 2 h. The resulting brown solution was concentrated to dryness in vacuo to afford the title compound, (2S,4S)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid trifluoroacetate as a brown oil (371.5 mg, 98%). LCMS (ESI): mass calcd for C₂₄H₂₆N₄O₅·C₂HF₃O₂, 450.2; m/z found, 451.1 [M+H]⁺.

### Intermediate 40: 4-Chloro-2-(trifluoromethyl)benzofuro[3,2-d]pyrimidine

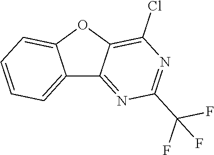 [SMILES: FC(F)(F)c1nc(Cl)c2oc3ccccc3c2n1]

Step A: N-(2-Cyanobenzofuran-3-yl)-2,2,2-trifluoroacetamide. To a solution of 3-aminobenzofuran-2-carbonitrile (20.0 g, 126 mmol, 1.00 eq) in pyridine (150 mL) was added TFAA (29.2 g, 139 mmol, 19.3 mL, 1.00 eq) at 0° C. The mixture was stirred at 25° C. for 12 hrs. The reaction mixture was quenched by addition of H₂O (100 mL at 25° C.), and then extracted with ethyl acetate (100 mL×3). The combined organic layers were washed with HCl (1 M, 100 mL×3), dried over Na₂SO₄, filtered and concentrated under reduced pressure to give a residue. The residue was purified (FCC, SiO₂, 0˜15% ethyl acetate/petroleum ether) to afford the title compound, N-(2-cyanobenzofuran-3-yl)-2,2,2-trifluoroacetamide (20.0 g, 62.2% yield) as an off-white solid. LCMS (ESI): mass calcd for C₁₁H₅F₃N₂O₂, 272.0; m/z found, 273.0 [M+H]⁺. ¹H NMR (400 MHz, DMSO-d₆) δ 12.72-12.15 (m, 1H), 7.93 (d, J=7.60 Hz, 1H), 7.79-7.73 (m, 1H), 7.70-7.63 (m, 1H), 7.52-7.45 (m, 1H).

Step B. 4-Chloro-2-(trifluoromethyl)benzofuro[3,2-d]pyrimidine. To a solution of N-(2-cyanobenzofuran-3-yl)-2,2,2-trifluoroacetamide (15.0 g, 60.5 mmol, 1.00 eq) in sulfolane (100 mL) was added POCl₃ (92.4 g, 605 mmol, 56.4 mL, 10.0 eq) at 25° C. The mixture was stirred at 130° C. for 48 hrs. The reaction mixture was quenched by addition of H₂O (200 mL) at 0° C., and then extracted with Ethyl acetate (200 mL×3). The combined organic layers were washed with brine (40.0 mL×4), dried over Na₂SO₄, filtered and concentrated under reduced pressure to give a residue. The residue was purified (FCC, SiO₂, 0˜10% Ethyl acetate/Petroleum ether) to afford the title compound, 4-chloro-2-(trifluoromethyl)benzofuro[3,2-d]pyrimidine (12.0 g, 72.7% yield) as an off-white solid. LCMS (ESI): mass calcd for C₁₁H₄ClF₃NO, 272.0; m/z found, 273.0 [M+H]⁺. ¹H NMR (400 MHz, DMSO-d₆) δ 8.34 (d, J=7.60 Hz, 1H), 8.05 (d, J=8.40 Hz, 1H), 7.99-7.89 (m, 1H), 7.67 (t, J=7.60 Hz, 1H).

### Intermediate 41: (2S,4S)-4-((5-Bromo-3-((S*)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid

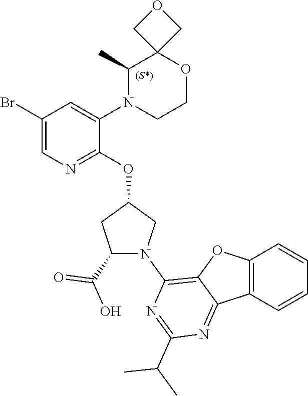

The title compound was prepared in a manner analogous to Intermediate 29, using 8-(5-bromo-2-nitropyridin-3-yl)-9-methyl-2,5-dioxa-8-azaspiro[3.5]nonane (Intermediate 10) instead of (S)-8-(5-bromo-2-nitropyridin-3-yl)-9-methyl-2,5-dioxa-8-azaspiro[3.5]nonane (Intermediate 17, product from Step A). LCMS (ESI): mass calcd for C₂₄H₂N₄O₅·C₂HF₃O₂, 637.2; m/z found, 638.1 [M+H]⁺. ¹H NMR (400 MHz, DMSO-d₆) δ=8.08 (d, J=8.0 Hz, 1H), 7.90 (d, J=2.0 Hz, 1H), 7.73-7.61 (m, 2H), 7.50-7.42 (m, 1H), 7.31 (d, J=2.0 Hz, 1H), 5.72-5.53 (m, 1H), 5.22-4.96 (m, 1H), 4.67-4.61 (m, 1H), 4.57-4.48 (m, 2H), 4.37-4.28 (m, 2H), 4.26-4.16 (m, 1H), 4.13-4.07 (m, 1H), 3.82-3.72 (m, 1H), 3.68-3.60 (m, 1H), 3.29-3.25 (m, 1H), 3.00-2.91 (m, 2H), 2.77-2.69 (m, 1H), 2.59-2.53 (m, 1H), 1.32 (dd, J=2.0, 6.8 Hz, 6H), 0.84 (d, J=6.4 Hz, 3H).

### Intermediate 42: (2S,4S)-4-((5-Bromo-3-((S*)-9-methyl-2,5-dioxa-8-azaspiro[3.5]nonan-8-yl)pyridin-2-yl)oxy)-1-(2-(trifluoromethyl)benzofuro[3,2-d]pyrimidin-4-yl)pyrrolidine-2-carboxylic acid

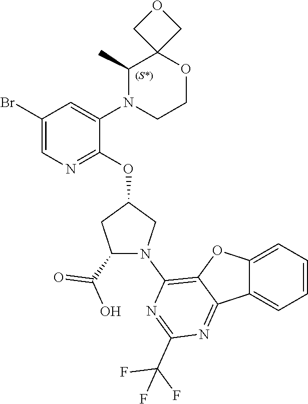

(2S,4S)-4-((5-Bromo-3-((S*)-9-methyl-2,5-dioxa-8-azaspiro[3.5]nonan-8-yl)pyridin-2-yl)oxy)-1-(2-(trifluoromethyl)benzofuro[3,2-d]pyrimidin-4-yl)pyrrolidine-2-carboxylic acid. A mixture of (2S,4S)-4-((5-bromo-3-((S*)-9-methyl-2,5-dioxa-8-azaspiro[3.5]nonan-8-yl)pyridin-2-yl)oxy)pyrrolidine-2-carboxylic acid trifluoroacetate (Intermediate 10, 500.0 mg, 0.9220 mmol), DIEA (0.4 mL, 2 mmol), 4-chloro-2-(trifluoromethyl)benzofuro[3,2-d]pyrimidine (Intermediate 40, 180.0 mg, 0.6603 mmol) and DMSO (5.0 mL) was stirred at 110° C. for 2 h, and cooled to rt. The reaction mixture was diluted with H₂O (10.0 mL), aq HCl (1 M) was added until the pH measured 5. The reaction mixture was extracted with EtOAc (15.0 mL×2), and the combined extracts dried over anhydrous Na₂SO₄, filtered, and concentrated to dryness in vacuo to give a yellow oil. The resulting oil was purified (FCC, SiO₂, 0-100% EtOAc/pet ether) to afford the title compound (2S,4S)-4-((5-bromo-3-((S*)-9-methyl-2,5-dioxa-8-azaspiro[3.5]nonan-8-yl)pyridin-2-yl)oxy)-1-(2-(trifluoromethyl)benzofuro[3,2-d]pyrimidin-4-yl)pyrrolidine-2-carboxylic acid as a yellow solid (300.0 mg, 66%). LCMS (ESI): mass calcd for C₂₈H₂₅BrF₃N₅O, 663.1; m/z found, 664.3 [M+H]⁺. ¹H NMR (400 MHz, CDCl₃) δ=8.25 (d, J=8.0 Hz, 1H), 7.87 (d, J=2.0 Hz, 1H), 7.67-7.58 (m, 2H), 7.50-7.44 (m, 1H), 7.14 (s, 1H), 6.27-6.21 (m, 1H), 5.71-5.40 (m, 1H), 5.02-4.93 (m, 1H), 4.86-4.73 (m, 1H), 4.69-4.45 (m, 4H), 4.12-4.05 (m, 1H), 3.92-3.83 (m, 1H), 3.66-3.55 (m, 1H), 3.30-3.18 (m, 1H), 2.96-2.73 (m, 2H), 2.68-2.54 (m, 1H), 0.92 (d, J=6.8 Hz, 3H).

### Intermediate 43. (2S,4S)-1-(2-Ethylbenzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid

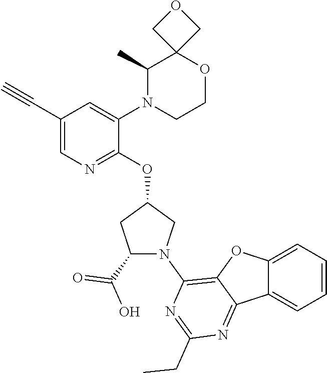 [SMILES: C#Cc1cnc(O[C@H]2C[C@@H](C(=O)O)N(c3nc(CC)nc4c3oc3ccccc34)C2)c(N2CCOC3(COC3)[C@@H]2C)c1]

Step A. (S)-8-(5-Bromo-2-fluoropyridin-3-yl)-9-methyl-2,5-dioxa-8-azaspiro[3.5]nonane. To a solution of (S)-8-(5-bromo-2-nitropyridin-3-yl)-9-methyl-2,5-dioxa-8-azaspiro[3.5]nonane (Intermediate 17, product from Step A, 15.0 g, 43.5 mmol) in DMF (150 mL) was added TBAF (1.00 M, 87.1 mL) dropwise at 0-5° C. The reaction mixture was stirred at 50° C. for 12 h. The reaction mixture was cooled to 25° C., and the resulting residue was poured into water (500 mL). The aqueous phase was extracted with ethyl acetate (50.0 mL×4). The combined organic phase was washed with brine (50.0 mL×5), separated, dried with anhydrous Na₂SO₄, filtered and concentrated in vacuum. The resulting residue was purified (FCC, SiO₂, Petroleum ether/Ethyl acetate=0% to 20%) to afford the title compound, (S)-8-(5-bromo-2-fluoropyridin-3-yl)-9-methyl-2,5-dioxa-8-azaspiro[3.5]nonane (12.0 g, 37.5 mmol, 86.5% yield) as an off white solid. LCMS (ESI): mass calcd for C₁₂H₁₄BrFN₂O₂, 316.0/318.0 m/z found, 318.8 [M+H]⁺. ¹H NMR (400 MHz, CDCl3) δ 7.82 (t, J=2.0 Hz, 1H), 7.29 (dd, J=9.2, 2.0 Hz, 1H), 4.82-4.69 (m, 2H), 4.53-4.37 (m, 2H), 4.20 (q, J=6.4 Hz, 1H), 3.94-3.85 (m, 1H), 3.73 (dt, J=11.6, 3.2 Hz, 1H), 3.34 (dt, J=11.6, 3.6 Hz, 1H), 2.76-2.68 (m, 1H), 1.01 (d, J=6.8 Hz, 3H)

Step B. (S)-8-(2-Fluoro-5-((trimethylsilyl)ethynyl)pyridin-3-yl)-9-methyl-2,5-dioxa-8-azaspiro[3.5]nonane. (9S)-8-(5-Bromo-2-fluoro-3-pyridyl)-9-methyl-2,5-dioxa-8-azaspiro[3.5]nonane (200.0 mg, 630.6 μmol), copper(I) iodide (15.61 mg, 81.98 μmol), bis(triphenylphosphine)palladium(ii) chloride (44.26 mg, 63.06 μmol) were subjected to three cycles of vacuum and recharging with N₂, tetrahydrofuran (45.47 mg, 4.204 mL, 0.15 molar, 630.6 μmol), trimethylsilylacetylene (92.91 mg, 131 μL, 945.9 μmol) were added, and the reaction stirred at room temperature for 20 h. Celite® was added to the reaction and the resulting suspension was concentrated in vacuo to dryness. Purification (FCC, SiO₂, solid load on Celite®, Isco gold, 0-50% EA in hex) afforded the title compound (175 mg, 523 μmol, 83.0%) as a light yellow film. LCMS (ESI): mass calcd for C₁₇H₂₃FN₂O₂Si 334.2, m/z found, 335.2 [M+H]⁺. ¹H NMR (500 MHz, CDCl₃) δ 7.91 (s, 1H), 7.31-7.27 (m, 1H), 4.78 (d, J=6.7 Hz, 1H), 4.74 (d, J=6.7 Hz, 1H), 4.49 (d, J=7.1 Hz, 1H), 4.41 (d, J=7.2 Hz, 1H), 4.20-4.15 (m, 1H), 3.89 (ddd, J=11.7, 3.7, 1.6 Hz, 1H), 3.74 (td, J=11.5, 3.2 Hz, 1H), 3.35 (td, J=11.6, 3.7 Hz, 1H), 2.72 (dt, J=11.9, 2.6 Hz, 1H), 0.98 (dd, J=15.1, 6.6 Hz, 3H), 0.26 (s, 9H).

Step C. (2S,4S)-1-(2-Ethylbenzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid. A mixture of (S)-8-(2-fluoro-5-((trimethylsilyl)ethynyl)pyridin-3-yl)-9-methyl-2,5-dioxa-8-azaspiro[3.5]nonane (175 mg, 523 μmol), (2S,4S)-1-(2-ethylbenzofuro[3,2-d]pyrimidin-4-yl)-4-hydroxy-pyrrolidine-2-carboxylic acid (171 mg, 523 μmol), was evacuated and refilled with N₂, then charged with N,N-dimethylformamide (38.2 mg, 2.62 mL, 0.2 molar, 523 μmol) and potassium bis(trimethylsilyl)amide (313 mg, 1.57 mL, 1.0 molar, 1.57 mmol) was added dropwise. The reaction mixture was stirred at rt for 5.25 h, then the reaction mixture was acidified with citric acid (1M) to pH -4. Aqueous workup with DCM resulted in poor extraction and formation of a white precipitate. The resulting precipitate and organic layers were combined, diluted with DCM/MeOH to solubilize and concentrated onto Celite® to dryness. Purification (FCC, SiO₂, 0-100% of (5% MeOH in DCM) in DCM) afforded the title compound (85 mg, 0.15 mmol, 29%) as a colorless solid. LCMS (ESI): mass calcd for C₃₁H₃₁N₅O₆ 569.2, m/z found, 570.6 [M+H]⁺. ¹H NMR (500 MHz, DMSO) δ 8.14-8.05 (m, 1H), 7.96 (d, J=1.9 Hz, 1H), 7.74-7.62 (m, 2H), 7.50-7.43 (m, 1H), 7.22 (d, J=2.0 Hz, 1H), 5.70 (s, 1H), 5.65 (s, 1H), 5.13 (s, 1H), 4.64 (d, J=6.5 Hz, 1H), 4.54 (d, J=6.6 Hz, 1H), 4.34 (d, J=6.8 Hz, 1H), 4.30-4.16 (m, 2H), 4.13-4.06 (m, 2H), 3.76 (ddd, J=11.5, 3.6, 1.7 Hz, 1H), 3.64 (td, J=11.4, 3.1 Hz, 1H), 3.25 (td, J=11.7, 3.7 Hz, 1H), 2.98 (ddd, J=14.3, 10.0, 6.1 Hz, 1H), 2.83 (q, J=7.5 Hz, 2H), 2.70 (d, J=12.0 Hz, 1H), 1.32 (t, J=7.6 Hz, 3H), 0.82 (d, J=6.5 Hz, 3H).

### Intermediate 44: (2S,4S)-1-(2-(4,4-Difluoropiperidin-1-yl)benzofuro[3,2-d]pyrimidin-4-yl)-4-hydroxypyrrolidine-2-carboxylic acid

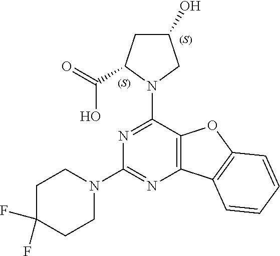

Step A. (2S,4S)-1-(2-Chlorobenzofuro[3,2-d]pyrimidin-4-yl)-4-hydroxypyrrolidine-2-carboxylic acid. A mixture of 2,4-dichlorobenzofuro[3,2-d]pyrimidine (2.00 g, 8.37 mmol), (2S,4S)-4-hydroxypyrrolidine-2-carboxylic acid (1.21 g, 9.20 mmol), DIEA (4.27 mL, 25.1 mmol), and ACN (20.0 mL) was refluxed at 80° C. for 12 h. The reaction mixture was cooled and concentrated to dryness under reduced pressure to afford brown oil. The resulting brown oil was poured into H₂O (30 mL), the pH was adjusted to 5 with 1 N HCl and the resulting suspension was isolated via filtration. The filter cake was washed with H₂O (20×3 mL) and the resulting solids were dried under reduced pressure to afford the title compound, (2S,4S)-1-(2-chlorobenzofuro[3,2-d]pyrimidin-4-yl)-4-hydroxypyrrolidine-2-carboxylic acid as a white solid (3.3 g, 77% purity, 91%). LCMS (ESI): mass calcd for C₁₅H₁₂ClN₃O₄, 333.1; m/z found, 334.0 [M+H]⁺.

Step B. (2S,4S)-1-(2-(4,4-Difluoropiperidin-1-yl)benzofuro[3,2-d]pyrimidin-4-yl)-4-hydroxypyrrolidine-2-carboxylic acid. A mixture of (2S,4S)-1-(2-chlorobenzofuro[3,2-d]pyrimidin-4-yl)-4-hydroxypyrrolidine-2-carboxylic acid (250.0 mg, 0.5791 mmol), 4,4-difluoropiperidine (155.0 mg, 1.280 mmol), DIEA (500 uL, 2.940 mmol) and DMSO (2.0 mL) was stirred at 110° C. for 2 days. The reaction mixture was directly purified by (HPLC:Boston prime C18, 5 μm, 150 mm×30 mm; 7 min gradient (23-53% ACN/H₂O (with 0.05% of 25% aq NH₃+10 mM NH₄HCOs)) at 25 mL/min)) to afford the title compound, (2S,4S)-1-(2-(4,4-difluoropiperidin-1-yl)benzofuro[3,2-d]pyrimidin-4-yl)-4-hydroxypyrrolidine-2-carboxylic acid as a white solid (110.0 mg, 45% yield). LCMS (ESI): mass calcd for C₂₀H₂₀F₂N₄O₄, 418.1; m/z found, 419.1 [M+H]⁺. ¹H NMR (400 MHz, CDCl₃) δ=8.06-7.99 (m, 1H), 7.52-7.43 (m, 2H), 7.33-7.28 (m, 1H), 4.87-4.75 (m, 1H), 4.60-4.51 (m, 1H), 4.47-4.31 (m, 1H), 4.08-3.96 (m, 41H), 3.60-3.52 (m, 1H), 3.03-2.92 (m, 1H), 2.41-2.29 (m, 21H), 2.02-1.92 (in, 41H).

### Intermediates 45, 59 and 60 in Table 5 were prepared in a manner analogous to Intermediate 44, using (2S,4S)-1-(2-chlorobenzofuro[3,2-d]pyrimidin-4-yl)-4-hydroxypyrrolidine-2-carboxylic acid and the appropriate amine instead of 4,4-difluoropiperidine in Step B

TABLE 5

| Int. # | Structure | amine | MS and ¹H-NMR |
|---|---|---|---|
| 45 | 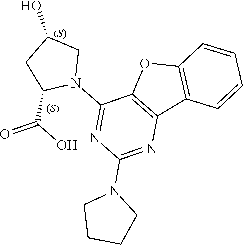 | pyrrolidine | LCMS (ESI): mass calcd for C₁₉H₂₀N₄O₄, 368.1; m/z found, 369.2 [M + H]⁺. ¹H NMR (400 MHz, DMSO-d₆) δ 7.94 (br d, J = 7.6 Hz, 1H), 7.73 − 7.49 (m, 2H), 7.42 − 7.32 (m, 1H), 4.68 − 4.52 (m, 1H), 4.48 − 4.21 (m, 2H), 3.91 − 3.75 (m, 1H), 3.55 − 3.43 (m, 4H), 2.49 − 2.40 (m, 2H), 1.93 − 1.85 (m, 4H). |
| | (2S,4S)-4-Hydroxy-1-(2-(pyrrolidin-1- | | |
| | yl)benzofuro[3,2-d]pyrimidin-4- | | |
| | yl)pyrrolidine-2-carboxylic acid | | |
| 59 | 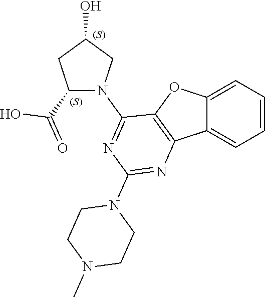 | 1-methyl piperazine | LCMS (ESI): mass calcd for C₂₀H₂₃N₅O₄, 397.2; m/z found, 398.2 [M + H]⁺. ¹H NMR (400 MHz, CD₃OD) δ ppm 2.09 (br d, J = 13.30 Hz, 1 H) 2.44 − 2.57 (m, 1 H) 2.72 − 2.95 (m, 4 H) 3.09 − 3.28 (m, 4 H) 3.79 (br s, 1 H) 3.86 − 4.09 (m, 2 H) 4.26 (br s, 2 H) 4.50 (br s, 2 H) 7.30 − 7.41 (m, 1 H) 7.53 − 7.62 (m, 2 H) 7.94 (d, J = 7.78 Hz, 1 H). |
| | (2S,4S)-4-hydroxy-1-(2-(4- | | |
| | methylpiperazin-1-yl)benzofuro[3,2- | | |
| | d]pyrimidin-4-yl)pyrrolidine-2- | | |
| | carboxylic acid | | |
| 60 | 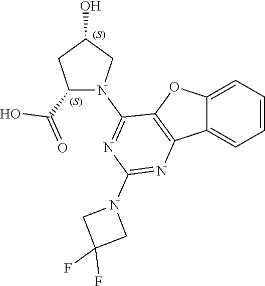 | 3,3-difluoroazetidine | LCMS (ESI): mass calcd for C₁₈H₁₆F₂N₄O₄, 390.1; m/z found, 391.1 [M + H]⁺. ¹H NMR (400 MHz, DMSO-d₆) δ 8.00 − 7.88 (m, 1H), 7.78 − 7.52 (m, 2H), 7.44 − 7.34 (m, 1H), 4.51 − 4.30 (m, 5H), 4.28 − 3.99 (m, 2H), 3.82 − 3.66 (m, 1H), 2.35 − 2.21 (m, 1H), 2.16 − 1.79 (m, 1H). |
| | (2S,4S)-1-(2-(3,3-Difluoroazetidin-1- | | |
| | yl)benzofuro[3,2-d]pyrimidin-4-yl)-4- | | |
| | hydroxypyrrolidine-2-carboxylic acid | | |

### Intermediate 46: 4-Chloro-2,6-bis(difluoromethyl)benzofuro[3,2-d]pyrimidine

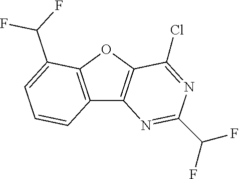 [SMILES: FC(F)c1nc(Cl)c2oc3c(C(F)F)cccc3c2n1]

Step A. 3-Amino-7-bromobenzofuran-2-carbonitrile. A mixture of 3-bromo-2-hydroxybenzonitrile (3.50 g, 17.7 mmol), 2-chloroacetonitrile (1.46 g, 19.3 mmol), K₂CO₃ (7.30 g, 52.8 mmol) and DMF (40.0 mL) was stirred at 100° C. overnight. The resulting brown heterogeneous reaction mixture was cool to rt, diluted with H₂O (60 mL), extracted with ethyl acetate (100 mL×3), dried over anhydrous Na₂SO₄, filtered and concentrated to dryness in vacuo to give a yellow oil. The resulting yellow oil was purified (FCC, SiO₂, 0-100% EtOAc/pet ether) to afford the title compound, 3-amino-7-bromobenzofuran-2-carbonitrile as a yellow solid (3.00 g, 61%). LCMS (ESI): mass calcd for C₉H₅BrN₂O, 236.0; m/z found, 236.9 [M+H]⁺. ¹H NMR (400 MHz, DMSO-d₆) δ 7.95-7.91 (m, 1H), 7.79-7.76 (m, 1H), 7.30-7.24 (m, 1H), 6.82 (s, 2H).

Step B. N-(7-bromo-2-cyanobenzofuran-3-yl)-2,2-difluoroacetamide. A mixture of 3-amino-7-bromobenzofuran-2-carbonitrile (1.00 g, 3.021 mmol), 2,2-difluoroacetic anhydride (716.0 mg, 4.114 mmol) and pyridine (15 mL) was stirred at rt for 2 h. The resulting yellow homogeneous reaction mixture was diluted with H₂O (20 mL), extracted with ethyl acetate (30 mL×3). The combined extracts were dried over anhydrous Na₂SO₄, filtered and concentrated to dryness in vacuo to give a brown oil. The resulting oil was purified (FCC, SiO₂, 0-30% ethyl acetate/petroleum ether) to afford the title compound, N-(7-bromo-2-cyanobenzofuran-3-yl)-2,2-difluoroacetamide as a yellow solid (900 mg, 88%). LCMS (ESI): mass calcd for C₁₁H₅BrF₂N₂O₂, 314.0; m/z found, 314.9 [M+H]⁺. ¹H NMR (400 MHz, DMSO-d₆) δ 11.83 (s, 1H), 8.01-7.96 (m, 1H), 7.94-7.90 (m, 1H), 7.46-7.41 (m, 1H), 6.60 (t, J=53.2 Hz, 1H).

Step C. 6-Bromo-2-(difluoromethyl)benzofuro[3,2-d]pyrimidin-4-ol. A mixture of N-(7-bromo-2-cyanobenzofuran-3-yl)-2,2-difluoroacetamide (900.0 mg, 2.66 mmol), ethanol (5.0 mL), H₂O (1.00 mL), 30% hydrogen peroxide solution (1.60 mL, 15.7 mmol) and NaOH (190.0 mg, 4.750 mmol) was cooled to 0° C. then subjected to vacuum and recharging with N₂ three times. The yellow heterogeneous reaction mixture was stirred at 80° C. for 2 h. The reaction mixture was quenched with saturated aq. Na₂SO₃ (10 mL), diluted with H₂O (20 mL), extracted with EtOAc (30 mL×3). The combined extracts were dried over anhydrous Na₂SO₄, filtered, and concentrated to dryness in vacuo to give a yellow solid, which was purified (FCC, SiO₂, 0-100% EtOAc/pet ether) to afford the title compound, 6-bromo-2-(difluoromethyl)benzofuro[3,2-d]pyrimidin-4-ol as a yellow solid (700 mg, 81%). LCMS (ESI): mass calcd for C₁₁H₅BrF₂N₂O₂, 314.0; m/z found, 314.9 [M+H]⁺. ¹H NMR (400 MHz, DMSO-d₆) δ 14.04-13.80 (m, 1H), 8.13-8.10 (m, 1H), 8.00-7.96 (m, 1H), 7.52-7.46 (m, 1H), 6.95 (t, J=53.2 Hz, 1H).

Step D. 2-(Difluoromethyl)-6-vinylbenzofuro[3,2-d]pyrimidin-4-ol. A mixture of 6-bromo-2-(difluoromethyl)benzofuro[3,2-d]pyrimidin-4-ol (690.0 mg, 2.111 mmol), 4,4,5,5-tetramethyl-2-vinyl-1,3,2-dioxaborolane (414.0 mg, 2.688 mmol), dioxane:H₂O (9:1, 15.0 mL), K₂CO₃ (897.0 mg, 6.490 mmol), and Pd(dppf)Cl₂ (158.0 mg, 0.2160 mmol) was purged with nitrogen and heated at 100° C. for 1 h. The reaction mixture was cooled, diluted with H₂O (15 mL), and extracted with ethyl acetate (30 mL×3). The combined extracts dried over anhydrous Na₂SO₄, filtered and concentrated to dryness in vacuo to give a black solid. The resulting residue was purified (FCC, SiO₂, 0-100% EtOAc/pet ether) to afford the title compound, 2-(difluoromethyl)-6-vinylbenzofuro[3,2-d]pyrimidin-4-ol as a yellow solid (600.0 mg, 80%). LCMS (ESI): mass calcd for C₁₃H₈F₂N₂O₂, 262.1; m/z found, 262.9 [M+H]⁺. ¹H NMR (400 MHz, DMSO-d₆) δ 8.01 (d, J=8.0 Hz, 1H), 7.83 (d, J=7.2 Hz, 1H), 7.54 (d, J=7.6 Hz, 1H), 7.15-6.81 (m, 2H), 6.31 (d, J=17.6 Hz, 1H), 5.67 (d, J=11.2 Hz, 1H).

Step E. 2-(Difluoromethyl)-4-hydroxybenzofuro[3,2-d]pyrimidine-6-carbaldehyde. A mixture of 2-(difluoromethyl)-6-vinylbenzofuro[3,2-d]pyrimidin-4-ol (590.0 mg, 1.651 mmol), THF/H₂O (4/1, 15.0 mL), NalO₄ (1.70 g, 7.95 mmol) and K₂OsO₄·2H₂O (300.0 mg, 0.8140 mmol) were combined at 0° C. The resulting brown reaction mixture was stirred at rt for 4 h. The reaction mixture was treated with H₂O (10 mL), quenched with saturated aq. Na₂SO₃ (20 mL), filtered and concentrated to dryness in vacuo to give a yellow oil. The resulting residue was purified (HPLC: Xtimate C18, 10 μm, 150 mm×40 mm; 8 min gradient (5-35% ACN/H₂O (with 0.05% of 25% aq NH₃+10 mM NH₄HCO₃)) at 55 mL/min)) to afford the title compound, 2-(difluoromethyl)-4-hydroxybenzofuro[3,2-d]pyrimidine-6-carbaldehyde as a yellow solid (90.0 mg, 20%). LCMS (ESI): mass calcd for C₁₂H₆F₂N₂O₃, 264.0; m/z found, 265.0 [M+H]⁺. ¹H NMR (400 MHz, DMSO-d₆) δ 10.47 (s, 1H), 8.28 (d, J=1.2 Hz, 1H), 8.04-7.99 (m, 1H), 7.59-7.53 (m, 1H), 6.49 (t, J=55.6 Hz, 1H).

Step F. 2,6-Bis(difluoromethyl)benzofuro[3,2-d]pyrimidin-4-ol. At 0° C. was combined 2-(difluoromethyl)-4-hydroxybenzofuro[3,2-d]pyrimidine-6-carbaldehyde (85.0 mg, 0.314 mmol), DCM (1.0 mL) and DAST (84 uL, 0.63 mmol). The reaction mixture was subjected to three cycles of vacuum and recharging with nitrogen. The reaction mixture stirred at rt overnight. The reaction mixture was quenched with saturated aq. NaHCO₃ at 0° C., adjusted the pH to ˜8, and extracted with DCM (5 mL×3). The combined extracts were dried over anhydrous Na₂SO₄ and concentrated to dryness in vacuo to give a yellow oil. The resulting residue was purified (FCC, SiO₂, gradient elution: 0-30% EtOAc/DCM) to afford the title compound, 2,6-bis(difluoromethyl)benzofuro[3,2-d]pyrimidin-4-ol as a yellow solid (20.0 mg, 18% yield). LCMS (ESI): mass calcd for C₁₂H₆F₄N₂O₂, 286.0; m/z found, 287.0 [M+H]⁺.

Step G. 4-Chloro-2,6-bis(difluoromethyl)benzofuro[3,2-d]pyrimidine. A mixture of 2,6-bis(difluoromethyl)benzofuro[3,2-d]pyrimidin-4-ol (20.0 mg, 0.555 mmol), DIEA (24 uL, 0.14 mmol), 1,4-dioxane (0.5 mL), and POCl₃ (38 uL, 0.42 mmol) was stirred at 100° C. for 1 h. The reaction mixture was cooled to rt, and poured it into H₂O (10 mL). The resulting mixture was extracted with ethyl acetate (10 mL×3) and the combined extracts were dried over anhydrous Na₂SO₄, filtered, and concentrated to dryness in vacuo to give a yellow solid. The resulting residue was purified (FCC, SiO₂, 0-60% EtOAc/pet ether) to afford the title compound, 4-chloro-2,6-bis(difluoromethyl)benzofuro[3,2-d]pyrimidine as a yellow solid (15 mg, 66%). LCMS (ESI): mass calcd for C₁₂H₅ClF₄N₂O, 304.0; m/z found, 304.9 [M+H]⁺.

### Intermediate 47: 4-Chloro-2-(1,1,1-trifluoropropan-2-yl)benzofuro[3,2-d]pyrimidine

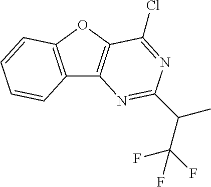 [SMILES: CC(c1nc(Cl)c2oc3ccccc3c2n1)C(F)(F)F]

Step A. N-(2-Cyanobenzofuran-3-yl)-3,3,3-trifluoro-2-methylpropanamide. A mixture of 3-aminobenzofuran-2-carbonitrile (330.0 mg, 2.087 mmol), Py (6 mL), 3,3,3-trifluoro-2-methylpropanoic acid (400.0 mg, 2.815 mmol), and EDC·HCl (840.0 mg, 4.382 mmol) was stirred at rt overnight. The reaction mixture was triturated with H₂O (20 mL) at rt for 10 min, and filtered. The filter cake was rinsed with H₂O (15 mL×2), and the resulting solid was dried in vacuo to give the title compound, N-(2-cyanobenzofuran-3-yl)-3,3,3-trifluoro-2-methylpropanamide as grey solid (540.0 mg, 91%). LCMS (ESI): mass calcd for C₁₃H₉F₃N₂O₂, 282.1; m/z found, 283.0 [M+H]⁺. ¹H NMR (400 MHz, DMSO-d₆) δ 11.19 (br s, 1H), 7.95 (d, J=8.0 Hz, 1H), 7.74-7.69 (m, 1H), 7.68-7.62 (m, 1H), 7.52-7.44 (m, 1H), 3.87-3.72 (m, 1H), 1.44 (d, J=7.0 Hz, 3H).

Step B. 2-(1,1,1-Trifluoropropan-2-yl)benzofuro[3,2-d]pyrimidin-4-ol. A mixture of N-(2-cyanobenzofuran-3-yl)-3,3,3-trifluoro-2-methylpropanamide (510.0 mg, 1.788 mmol), and phosphoric acid (12 mL, 33 mmol) was stirred at 135° C. overnight. The reaction mixture was cooled and added to water (15 mL), extracted with EtOAc (20 mL×2), and the combined extracts were concentrated to dryness in vacuo. The resulting residue was purified (FCC, SiO₂, 0-50% EtOAc/pet ether) to afford the title compound, 2-(1,1,1-trifluoropropan-2-yl)benzofuro[3,2-d]pyrimidin-4-ol as a yellow solid (53.0 mg, 10%). LCMS (ESI): mass calcd for C₁₃H₉F₃N₂O₂, 282.1; m/z found, 283.0 [M+H]⁺. ¹H NMR (400 MHz, DMSO-d₆) δ 13.17 (br s, 1H), 8.07 (d, J=7.7 Hz, 1H), 7.86 (d, J=8.5 Hz, 1H), 7.76-7.66 (m, 1H), 7.58-7.47 (m, 1H), 3.99-3.86 (m, 1H), 1.58 (d, J=7.0 Hz, 3H).

Step C. 4-Chloro-2-(1,1,1-trifluoropropan-2-yl)benzofuro[3,2-d]pyrimidine. A mixture of 2-(1,1,1-trifluoropropan-2-yl)benzofuro[3,2-d]pyrimidin-4-ol (53.0 mg, 0.186 mmol), 1,4-dioxane (2.0 mL), DIEA (70 uL, 0.412 mmol) and POCl₃ (71 uL, 0.778 mmol) was stirred at 100° C. for 1 h. The reaction mixture was cooled and concentrated to dryness in vacuo to give a brown oil, which was purified (FCC, SiO₂, 0-10% EtOAc/pet ether) to give the title compound, 4-chloro-2-(1,1,1-trifluoropropan-2-yl)benzofuro[3,2-d]pyrimidine a white solid (44.0 mg, 79%). LCMS (ESI): mass calcd for C₁₃H₈ClF₃N₂O, 300.0; m/z found, 301.0 [M+H]⁺.

### Intermediate 48: 4-Chloro-2-(3-methyltetrahydrofuran-3-yl)benzofuro[3,2-d]pyrimidine

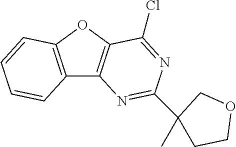 [SMILES: CC1(c2nc(Cl)c3oc4ccccc4c3n2)CCOC1]

Step A: N-(2-cyanobenzofuran-3-yl)-3-methyltetrahydrofuran-3-carboxamide. The title compound was prepared in a manner analogous to Intermediate 47, Step A, using 3-aminobenzofuran-2-carbonitrile and 3-methyltetrahydrofuran-3-carboxylic acid instead of 3,3,3-trifluoro-2-methylpropanoic acid. LCMS (ESI): mass calcd for C₁₅H₁₄N₂O₃, 270.1; m/z found, 271.1 [M+H]⁺. ¹H NMR (400 MHz, DMSO-d₆) δ 10.38 (s, 1H), 7.97 (d, J=7.7 Hz, 1H), 7.72-7.68 (m, 1H), 7.63 (dt, J=1.2, 7.8 Hz, 1H), 7.48-7.43 (m, 1H), 4.18 (d, J=8.7 Hz, 1H), 3.89-3.76 (m, 2H), 3.56 (d, J=8.7 Hz, 1H), 2.57-2.53 (m, 1H), 1.89 (ddd, J=6.3, 7.6, 12.5 Hz, 1H), 1.48 (s, 3H).

Step B. 2-(3-methyltetrahydrofuran-3-yl)benzofuro[3,2-d]pyrimidin-4-ol. A cooled solution, 0° C., of N-(2-cyanobenzofuran-3-yl)-3-methyltetrahydrofuran-3-carboxamide (120.0 mg, 0.4422 mmol), EtOH (1.00 mL), H₂O (0.20 mL), aq H₂O₂ (370.0 mg, 3.263 mmol, 30%) and NaOH (31.0 mg, 0.775 mmol) was subjected to vacuum and recharging with N₂ three times and stirred at 80° C. for 3 h. The reaction mixture was cooled to rt and quenched with aq. Na₂S₂O₃, diluted with H₂O (2 mL), and extracted with EtOAc (5 mL×2). The combined extracts were dried over anhydrous Na₂SO₄, filtered, and concentrated to dryness in vacuo to give a yellow solid. The resulting solid was purified (FCC, SiO₂, 0-50% EtOAc/pet ether) to afford the title compound, 2-(3-methyltetrahydrofuran-3-yl)benzofuro[3,2-d]pyrimidin-4-ol as white solid (70.0 mg, 57%). LCMS (ESI): mass calcd for C₁₅H₁₄N₂O₃, 270.1; m/z found, 271.1 [M+H]⁺. ¹H NMR (400 MHz, DMSO-d₆) δ 2.71 (br s, 1H), 8.04 (d, J=7.7 Hz, 1H), 7.84 (d, J=8.5 Hz, 1H), 7.68 (t, J=7.7 Hz, 1H), 7.53-7.47 (m, 1H), 4.25 (d, J=8.6 Hz, 1H), 3.93-3.80 (m, 2H), 3.73 (d, J=8.6 Hz, 1H), 2.64 (td, J=7.9, 12.2 Hz, 1H), 2.11-2.03 (m, 1H), 1.48 (s, 3H).

Step C: 4-Chloro-2-(3-methyltetrahydrofuran-3-yl)benzofuro[3,2-d]pyrimidine. The title compound was prepared in a manner analogous to Intermediate 47, Step C, using 2-(3-methyltetrahydrofuran-3-yl)benzofuro[3,2-d]pyrimidin-4-ol. LCMS (ESI): mass calcd for C₁₅H₁₃ClN₂O₂, 288.1; m/z found, 289.0 [M+H]⁺. ¹H NMR (400 MHz, DMSO-d₆) δ 8.28 (d, J=7.3 Hz, 1H), 7.99 (d, J=8.5 Hz, 1H), 7.91-7.85 (m, 1H), 7.65-7.59 (m, 1H), 4.25 (d, J=8.3 Hz, 1H), 4.01-3.85 (m, 2H), 3.78 (d, J=8.2 Hz, 1H), 2.81-2.65 (m, 1H), 2.14-2.01 (m, 1H), 1.54 (s, 3H).

### Intermediate 49: 8-(3,6-Dichloropyridazin-4-yl)-9-methyl-2,5-dioxa-8-azaspiro[3.5]nonane

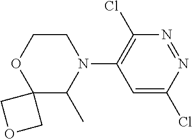 [SMILES: CC1N(c2cc(Cl)nnc2Cl)CCOC12COC2]

To a solution of 9-methyl-2,5-dioxa-8-azaspiro[3.5]nonane (35.0 g, 244 mmol) and 3,4,6-trichloropyridazine (56.0 g, 305. mmol) in NMP (350 mL) was added DIEA (63.1 g, 488 mmol, 85.1 mL) at 25° C. The reaction mixture was stirred at 80° C. for 12 hr. The reaction mixture was quenched with H₂O (200 mL) at 10° C., and extracted with ethyl acetate (200 mL×5). The combined organic layers were washed with brine (30.0 mL×4), separated, dried over Na₂SO₄, filtered and concentrated under reduced pressure. The resulting residue was purified (FCC, SiO₂, Eluent of 0-30% Ethyl acetate/Petroleum ether) afforded the title compound (61.5 g, 84.9% yield) as an off-white solid. LCMS (ESI): mass calcd for C₁₁H₁₃C₁₂N₃O₂ 289.0, m/z found, 290.1 [M+H]⁺. ¹H NMR (400 MHz, CDCl₃) δ 6.88 (s, 1H), 4.88-4.81 (m, 1H), 4.81-4.68 (m, 2H), 4.51 (d, J=7.20 Hz, 1H), 4.40 (d, J=7.20 Hz, 1H), 3.94 (dd, J=11.6, 3.20 Hz, 1H), 3.76 (td, J=11.6, 2.80 Hz, 1H), 3.45 (td, J=12.0, 3.60 Hz, 1H), 2.93-2.84 (m, 1H), 1.08 (d, J=6.40 Hz, 3H).

### Intermediate 50: 2-(2-Methyloxetan-2-yl)benzofuro[3,2-d]pyrimidin-4-yl trifluoromethanesulfonate

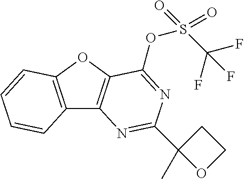 [SMILES: CC1(c2nc(OS(=O)(=O)C(F)(F)F)c3oc4ccccc4c3n2)CCO1]

Step A. N-(2-Cyanobenzofuran-3-yl)-2-methyloxetane-2-carboxamide. A mixture of 3-aminobenzofuran-2-carbonitrile (384.0 mg, 2.428 mmol), lithium 2-methyloxetane-2-carboxylate (300.0 mg, 2.458 mmol), Py (6.0 mL), and EDCl·HCl (768.0 mg, 4.006 mmol) was stirred at 60° C. overnight. The reaction mixture was concentrated to dryness in vacuo to give a black oil. The resulting residue was purified (FCC, SiO₂, 0-100% EtOAc/pet ether), followed by prep-TLC purification (1/2 EtOAc/pet ether) to give afford the title compound, N-(2-cyanobenzofuran-3-yl)-2-methyloxetane-2-carboxamide as a white solid (95 mg, 14%). LCMS (ESI): mass calcd for C₁₄H₁₂N₂O₃, 256.1; m/z found, 257.0 [M+H]⁺. ¹H NMR (400 MHz, CDCl₃) 9.12 (br s, 1H), 7.79 (d, J=8.0 Hz, 1H), 7.59-7.49 (m, 2H), 7.44-7.34 (m, 1H), 4.80-4.63 (m, 2H), 3.03-2.91 (m, 1H), 2.87-2.74 (m, 1H), 1.79 (s, 3H).

Step B. 2-(2-Methyloxetan-2-yl)benzofuro[3,2-d]pyrimidin-4-ol. N-(2-cyanobenzofuran-3-yl)-2-methyloxetane-2-carboxamide (95.0 mg, 0.351 mmol), EtOH/H₂O (1.5 mL, 5:1), 30% hydrogen peroxide solution (230.0 mg, 2.029 mmol) were combined at 0° C. (ice/water), and the reaction mixture was subjected to three cycles of vacuum and recharging with nitrogen. To the reaction mixture was added NaOH (28.0 mg, 0.700 mmol). The reaction mixture was stirred at 80° C. overnight, then cooled to rt. The reaction mixture was quenched with sat. aq. Na₂SO₃ (10 mL), treated with saturated aq. citric acid until the pH measured 6. The reaction mixture was extracted with DCM (20 mL×3) and the combined extracts were separated, dried over anhydrous Na₂SO₄, filtered and concentrated to dryness in vacuo to give a yellow solid. The resulting residue was purified (FCC, SiO₂, 0-100% EtOAc/per ether) to afford the title compound, 2-(2-methyloxetan-2-yl)benzofuro[3,2-d]pyrimidin-4-ol as a white solid (70 mg, 72%). LCMS (ESI): mass calcd for C₁₄H₁₂N₂O₃, 256.1; m/z found, 257.0 [M+H]⁺. ¹H NMR (400 MHz, CDCl₃) δ 10.39-10.29 (m, 1H), 8.07 (d, J=7.2 Hz, 1H), 7.71-7.66 (m, 1H), 7.65-7.59 (m, 1H), 7.48-7.42 (m, 1H), 4.83-4.70 (m, 1H), 4.69-4.59 (m, 1H), 2.99-2.91 (m, 2H), 1.92 (s, 3H).

Step C. 2-(2-Methyloxetan-2-yl)benzofuro[3,2-d]pyrimidin-4-yI trifluoromethanesulfonate. To a cooled, 0° C., mixture of 2-(3-methyloxetan-3-yl)benzofuro[3,2-d]pyrimidin-4-ol (70.0 mg, 0.252 mmol), DCM (3 mL), and Py (100 uL, 1.239 mmol) was added a solution of Tf₂O (150.0 mg, 0.5317 mmol) and DCM (0.5 mL) dropwise over 1 min at 0° C. The reaction mixture was stirred at 0° C. for 0.5 h. The reaction mixture was quenched with saturated aq. NaHCO₃ (3 mL) and extracted with DCM (15 mL×3). The combined extracts were separated, dried over anhydrous Na₂SO₄, filtered and concentrated to dryness in vacuo to give the title compound, 2-(2-methyloxetan-2-yl)benzofuro[3,2-d]pyrimidin-4-yl trifluoromethanesulfonate as a yellow sticky (100 mg crude, 102%) and was used without further purification. LCMS (ESI): mass calcd for C₁₅H₁₁F₃N₂O₅S, 388.0; m/z found, 389.1 [M+H]⁺.

Intermediates 55 and 56 in Table 6 were prepared in a manner analogous to Intermediate 50, using the appropriate acid instead of lithium 2-methyloxetane-2-carboxylate in Step A.

TABLE 6

| Int. | | | |
|---|---|---|---|
| # | Structure | acid | MS and ¹H-NMR |
| 55 | 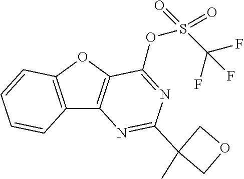 [SMILES: CC1(c2nc(OS(=O)(=O)C(F)(F)F)c3oc4ccccc4c3n2)COC1] | 3-methyloxetane-3- carboxylic acid | LCMS (ESI): mass calcd for C₁₅H₁₁F₃N₂O₅S, 388.0; m/z found, 389.1 [M + H]⁺. |
| | 2-(3-Methyloxetan-3- | | |
| | yl)benzofuro[3,2-d]pyrimidin-4-yl | | |
| | trifluoromethanesulfonate | | |
| 56 | 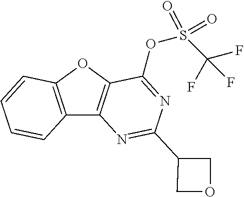 [SMILES: O=S(=O)(Oc1nc(C2COC2)nc2c1oc1ccccc12)C(F)(F)F] | 3-oxetane-3-carboxylic acid | LCMS (ESI): mass calcd for C₁₄H₉F₃N₂O₅S, 374.0; m/z found, 374.9 [M + H]⁺. ¹H NMR (400 MHz, CDCl₃) δ = 8.29 (d, J = 7.6 Hz, 1H), 7.85 − 7.75 (m, 2H), 7.62 − 7.56 (m, 1H), 5.18 − 5.08 (m, 4H), 4.78 − 4.67 (m, 1H). |
| | 2-(Oxetan-3-yl)benzofuro[3,2- | | |
| | d]pyrimidin-4-yl | | |
| | trifluoromethanesulfonate | | |

### Intermediate 51: (R)-4-chloro-2-(tetrahydrofuran-3-yl)benzofuro[3,2-d]pyrimidine

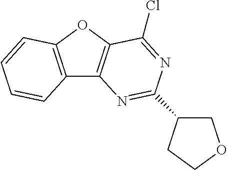 [SMILES: Clc1nc([C@H]2CCOC2)nc2c1oc1ccccc12]

Step A. (S)—N-(2-Cyanobenzofuran-3-yl)tetrahydrofuran-3-carboxamide. A mixture of 3-aminobenzofuran-2-carbonitrile (300.0 mg, 1.897 mmol), (R)-tetrahydrofuran-3-carboxylic acid (264.3 mg, 2.276 mmol), PY (3.0 mL), and EDCI (545.4 mg, 2.845 mmol) was stirred at rt for 12 h. The reaction mixture was then concentrated to dryness in vacuo to give brown oil. The resulting oil was purified (eluent: PE:EA=1:0 to 2:1) to afford the title compound, (S)—N-(2-cyanobenzofuran-3-yl)tetrahydrofuran-3-carboxamide as white solid (436 mg, >97% purity, 89%). LCMS (ESI): mass calcd for C₁₄H₁₂N₂O₃, 256.1; m/z found, 257.1 [M+H]⁺.

Step B. (R)-2-(tetrahydrofuran-3-yl)benzofuro[3,2-d]pyrimidin-4-ol. To a cooled, 0° C., mixture of (S)—N-(2-cyanobenzofuran-3-yl)tetrahydrofuran-3-carboxamide (200.0 mg, 0.7800 mmol), EtOH (2.0 mL), and H₂O (0.4 mL) under atmosphere of nitrogen, was added dropwise H₂O₂ (700.0 mg, 6.174 mmol) over 1 min at 0° C. To the reaction mixture was added NaOH (54.4 mg, 1.33 mmol) at 0° C. The reaction mixture was stirred at 80° C. for 2 h. The reaction mixture was cooled to rt and treated with aq Na₂SO₃ until the KI starch test paper indicated the reaction was quenched completely. The reaction mixture was extracted with ethyl acetate (20 mL×3) and the combined extracts were dried over anhydrous Na₂SO₄, filtered, and concentrated to dryness in vacuo to give a yellow oil. The resulting residue was purified (FCC, SiO₂, 30-100% EtOAc/pet ether) to give afford the title compound, (R)-2-(tetrahydrofuran-3-yl)benzofuro[3,2-d]pyrimidin-4-ol as a white solid (142 mg, 84% purity, 60%). LCMS (ESI): mass calcd for C₁₄H₁₂N₂O₃, 256.1; m/z found, 257.1 [M+H]⁺. ¹H NMR (400 MHz, DMSO-d₆) δ=8.02 (d, J=7.6 Hz, 1H), 7.80 (d, J=8.4 Hz, 1H), 7.70-7.62 (m, 1H), 7.52-7.43 (m, 1H), 4.08-4.01 (m, 1H), 3.96-3.90 (m, 2H), 3.81-3.76 (m, 1H), 3.55-3.52 (m, 1H), 2.39-2.21 (m, 2H).

Step C. (R)-4-chloro-2-(tetrahydrofuran-3-yl)benzofuro[3,2-d]pyrimidine. To a mixture of (R)-2-(tetrahydrofuran-3-yl)benzofuro[3,2-d]pyrimidin-4-ol (130.0 mg, 507.3 μmol), DIEA (0.17 mL, 1.0 mmol) and 1,4-dioxane (2.0 mL) was added POCl₃ (0.19 mL, 2.0 mmol) dropwise. The reaction mixture was stirred at rt for 1 h and turned yellow homogeneous. The reaction mixture was poured into H₂O (20 mL) and extracted with ethyl acetate (10 mL×3). The combined organic extracts were separated, washed with brine (10 mL×2), dried over anhydrous Na₂SO₄, filtered, and concentrated to dryness in vacuo to give brown oil. The resulting residue was purified (FCC, SiO₂, eluent: PE:EA=20:1 to 3:1) to afford the title compound, (R)-4-chloro-2-(tetrahydrofuran-3-yl)benzofuro[3,2-d]pyrimidine as white solid (57 mg, 91% purity, 37%). LCMS (ESI): mass calcd for C₁₄H₁₁ClN₂O₂, 274.1; m/z found, 274.9 [M+H]⁺.

Intermediates 52-54, and 57 in Table 7 were prepared in a manner analogous to Intermediate 51, using 3-aminobenzofuran-2-carbonitrile and the appropriate acid in Step A.

TABLE 7

| Int. # | Structure | acid | MS and 1H-NMR |
|---|---|---|---|
| 52 | 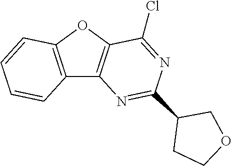 [SMILES: Clc1nc([C@@H]2CCOC2)nc2c1oc1ccccc12] | (R)- tetrahydrofuran- 3-carboxylic acid | LCMS (ESI): mass calcd for C₁₄H₁₁CIN₂O₂, 274.1; m/z found, 274.9 [M + H]⁺. |
| | (S)-4-Chloro-2-(tetrahydrofuran- | | |
| | 3-yl)benzofuro[3,2-d]pyrimidine | | |
| 53 | 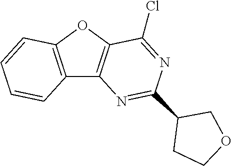 [SMILES: Clc1nc([C@@H]2CCOC2)nc2c1oc1ccccc12] | (S)- tetrahydrofuran- 2-carboxylic acid | LCMS (ESI): mass calcd for C₁₄H₁₁lIN₂O₂, 274.1; m/z found, 275.0 [M + H]⁺. ¹H NMR (400 MHz, CDCl₃) δ = 8.30 (d, J = 8.0 Hz, 1H), 7.78 − 7.70 (m, 2H), 7.56 − 7.50 (m, 1H), 5.31 − 5.26 (m, 1H), 4.35 − 4.27 (m, 1H), 4.13 − 4.04 (m, 1H), 2.54 − 2.44 (m, 1H), 2.31 − 2.14 (m, 2H), 2.12 − 2.03 (m, 1H). |
| | (S)-4-Chloro-2-(tetrahydrofuran- | | |
| | 2-yl)benzofuro[3,2-d]pyrimidine | | |
| 54 | 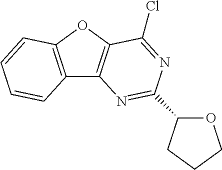 [SMILES: Clc1nc([C@H]2CCCO2)nc2c1oc1ccccc12] | (R)- tetrahydrofuran- 2-carboxylic acid | LCMS (ESI): mass calcd for C₁₄H₁₁ClN₂O₂, 274.1; m/z found, 275.1 [M + H]⁺. ¹H NMR (400 MHz, CDCl₃) δ = 8.31 (d, J = 7.6 Hz, 1H), 7.79 − 7.68 (m, 2H), 7.56 − 7.50 (m, 1H), 5.31 − 5.25 (m, 1H), 4.36 − 4.27 (m, 1H), 4.11 − 4.03 (m, 1H), 2.57 − 2.43 (m, 1H), 2.31 − 2.14 (m, 2H), 2.12 − 2.03 (m, 1H). |
| | (R)-4-chloro-2-(tetrahydrofuran- | | |
| | 2-yl)benzofuro[3,2-d]pyrimidine | | |
| 57 | 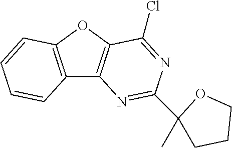 [SMILES: CC1(c2nc(Cl)c3oc4ccccc4c3n2)CCCO1] | 2- methyltetrahydrofuran- 2- carboxylic acid | LCMS (ESI): mass calcd for C₁₅H₁₃ClN₂O₂, 288.1; m/z found, 288.6 [M + H]⁺. ¹H NMR (400 MHz, DMSO-d₆) δ 8.31 − 8.24 (m, 1H), 7.98 (d, J = 8.4 Hz, 1H), 7.90 − 7.84 (m, 1H), 7.65 − 7.58 (m, 1H), 4.01 − 3.88 (m, 2H), 2.64 − 2.57 (m, 1H), 2.07 − 1.91 (m, 3H), 1.66 (s, 3H). |
| | 4-Chloro-2-(2- | | |
| | methyltetrahydrofuran-2- | | |
| | yl)benzofuro[3,2-d]pyrimidine | | |

### Intermediate 58: (S*)-9Methyl-8-(5-((1-methyl-H-pyrazol-3-yl)ethynyl)-2-nitropyridin-3-yl)-2,5-dioxa-8-azaspiro[3.5]nonane

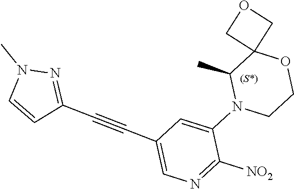

Step A. 8-(5-Bromo-2-nitropyridin-3-yl)-9-methyl-2,5-dioxa-8-azaspiro[3.5]nonane. The title compound was prepared in a manner analogous to Intermediate 17, Step A. LCMS (ESI): mass calcd for C₁₂H₁₄BrN₃O₄, 343.0; m/z found, 344.0 [M+H]⁺. ¹H NMR (400 MHz, DMSO-d₆) δ 8.32 (d, J=1.7 Hz, 1H), 8.31-8.29 (m, 1H), 4.58 (d, J=6.9 Hz, 1H), 4.45-4.36 (m, 2H), 4.28 (d, J=7.2 Hz, 1H), 3.82-3.70 (m, 2H), 3.63-3.54 (m, 1H), 3.34-3.26 (m, 1H), 2.77-2.70 (m, 1H), 0.97 (d, J=6.4 Hz, 3H).

Step B. 9-Methyl-8-(5-((1-methyl-1H-pyrazol-3-yl)ethynyl)-2-nitropyridin-3-yl)-2,5-dioxa-8-azaspiro[3.5]nonane. A mixture of 3-ethynyl-1-methyl-1H-pyrazole (180 mg, 1.696 mmol), 8-(5-bromo-2-nitropyridin-3-yl)-9-methyl-2,5-dioxa-8-azaspiro[3.5]nonane (700 mg, 1.604 mmol), Cs₂CO₃ (1.5 g, 4.604 mmol), Xphos Pd G3 (170 mg, 0.2008 mmol) Xphos (90 mg, 0.189 mmol) and MeCN (14 mL), under nitrogen, was stirred at 70° C. for 2 hours. The reaction mixture was cooled to rt and filtered over Celite®. The filter cake was rinsed with EtOAc (20 mL) and the filtrate was concentrated to dryness in vacuo. The resulting residue was purified (FCC, SiO₂, 0-100% EtOAc/pet ether) to afford the title compound, 9-methyl-8-(5-((1-methyl-1H-pyrazol-3-yl)ethynyl)-2-nitropyridin-3-yl)-2,5-dioxa-8-azaspiro[3.5]nonane (800 mg) as yellow solid.

Step C: (S*)-9-Methyl-8-(5-((1-methyl-1H-pyrazol-3-yl)ethynyl)-2-nitropyridin-3-yl)-2,5-dioxa-8-azaspiro[3.5]nonane. 9-Methyl-8-(5-((1-methyl-1H-pyrazol-3-yl)ethynyl)-2-nitropyridin-3-yl)-2,5-dioxa-8-azaspiro[3.5]nonane (800.0 mg crude, 2.166 mmol) was purified by SFC (DAICEL CHIRALPAK AD, 10 μm, 250 mm×30 mm; isocratic (35% EtOH (containing 0.1% of 25% aq NH₃)/CO₂) at 150 mL/min (100 bar); column temp 35° C.) to give two products: 1st Eluting: (R*)-9-methyl-8-(5-((1-methyl-1H-pyrazol-3-yl)ethynyl)-2-nitropyridin-3-yl)-2,5-dioxa-8-azaspiro[3.5]nonane (Rt=3.055 min, 320.0 mg, 40%); and the title compound as the 2nd Eluting: (S*)-9-methyl-8-(5-((1-methyl-1H-pyrazol-3-yl)ethynyl)-2-nitropyridin-3-yl)-2,5-dioxa-8-azaspiro[3.5]nonane: (Rt=3.545 min, 260.0 mg, 32%). LCMS (ESI): mass calcd for C18H₁₉N₅O₄, 369.1; m/z found, 370.0 [M+H]⁺.

### Intermediate 61: (2S,4S)-1-(2-Ethylbenzofuro[3,2-d]pyrimidin-4-yl)-4-hydroxypyrrolidine-2-carboxylic acid

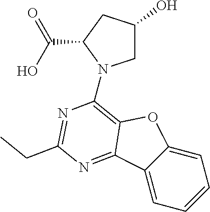 [SMILES: CCc1nc(N2C[C@@H](O)C[C@H]2C(=O)O)c2oc3ccccc3c2n1]

A solution of 4-chloro-2-ethylbenzofuro[3,2-d]pyrimidine (Intermediate 18, 600.0 mg, 2.532 mmol), (2S,4S)-4-hydroxypyrrolidine-2-carboxylic acid (335.0 mg, 2.555 mmol), DMSO (10.0 mL), and DIEA (0.95 mL, 5.6 mmol) was heated 110° C. for 2 h. The reaction mixture was cooled and purified directly (HPLC, Phenomenex Genimi NX C18, 5 μm, 150 mm×40 mm; 8 min gradient (4-34% ACN/H₂O (with 0.2% FA)) at 55 mL/min) to afford (2S,4S)-1-(2-ethylbenzofuro[3,2-d]pyrimidin-4-yl)-4-hydroxypyrrolidine-2-carboxylic acid as a white solid (690.0 mg, 83%). LCMS (ESI): mass calcd for C₁₇H₁₇N₃O₄ 327.1 m/z found, 328.1 [M+H]⁺. ¹H NMR (400 MHz, DMSO-d₆) δ 12.61 (br s, 1H), 8.07 (d, J=7.6 Hz, 1H), 7.87-7.59 (m, 2H), 7.55-7.40 (m, 1H), 5.30-4.98 (m, 1H), 4.77-4.43 (m, 1H), 4.39-3.88 (m, 1H), 3.87-3.67 (m, 1H), 3.30-3.23 (m, 1H), 2.88-2.71 (m, 2H), 2.48-2.37 (m, 1H), 2.31-1.87 (m, 1H), 1.39-1.17 (m, 3H).

### Intermediate 62: (2S,4S)-4-((6-Chloro-4-((S*)-9-methyl-2,5-dioxa-8-azaspiro[3.5]nonan-8-yl)pyridazin-3-yl)oxy)-1-(2-(difluoromethyl)benzofuro[3,2-d]pyrimidin-4-yl)pyrrolidine-2-carboxylic acid

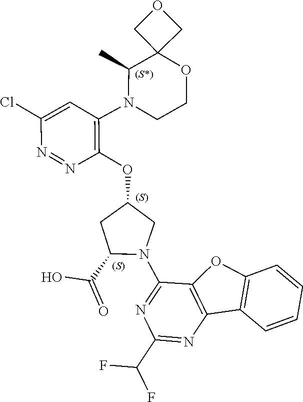

To a solution of (2S,4S)-1-(2-(difluoromethyl)benzofuro[3,2-d]pyrimidin-4-yl)-4-hydroxypyrrolidine-2-carboxylic acid (Intermediate 9, 50.0 g, 123 mmol) in THF (400 mL) was added t-BuOK (41.4 g, 369 mmol, 55.8 mL) at 0° C. The reaction mixture was stirred at 0° C. for 0.5 hr. Then 8-(3,6-dichloropyrdazin-4-yl)-9-methyl-2,5-dioxa-8-azaspiro[3.5]nonane (Intermediate 49, 42.8 g, 147 mmol) in THF (100 mL) was added at 0° C. The reaction mixture was stirred at 25° C. for 4.5 hrs and then quenched by the addition of NH₄Cl (300 mL) at 0° C., and the pH was adjusted to 5-6 with 1 M hydrochloric acid, diluted with H₂O 500 mL, and extracted with ethyl acetate (400 mL×3). The combined organic layers were washed with brine 50.0 mL, dried over Na₂SO₄, filtered and concentrated under reduced pressure. The resulting residue was purified (FCC, SiO₂, Eluent of 0-100% Ethyl acetate/Petroleum ether gradient @100 mL/min) to afford (2S,4S)-4-((6-chloro-4-((R*)-9-methyl-2,5-dioxa-8-azaspiro[3.5]nonan-8-yl)pyridazin-3-yl)oxy)-1-(2-(difluoromethyl)benzofuro[3,2-d]pyrimidin-4-yl)pyrrolidine-2-carboxylic acid (21.8 g, 29.6%); and the title compound, (2S,4S)-4-((6-chloro-4-((S*)-9-methyl-2,5-dioxa-8-azaspiro[3.5]nonan-8-yl)pyridazin-3-yl)oxy)-1-(2-(difluoromethyl)benzofuro[3,2-d]pyrimidin-4-yl)pyrrolidine-2-carboxylic acid (20.5 g, 28.3%): LCMS (ESI): mass calcd for C₂₇H₂₅ClF₂N₆O₆ 602.2, m/z found, 603.2 [M+H]⁺.

¹H NMR (400 MHz, DMSO-d₆) δ 13.18-12.02 (m, 1H), 8.15 (d, J=7.60 Hz, 1H), 7.83-7.68 (m, 2H), 7.53 (t, J=7.60 Hz, 1H), 6.96 (s, 1H), 6.93-6.60 (m, 1H), 5.82 (br s, 1H), 5.49-5.15 (m, 1H), 4.63 (br d, J=6.40 Hz, 1H), 4.53 (br d, J=6.80 Hz, 2H), 4.46 (br d, J=6.40 Hz, 1H), 4.35 (br d, J=6.80 Hz, 2H), 4.15 (d, J=6.80 Hz, 1H), 3.78 (br dd, J=11.6, 2.40 Hz, 1H), 3.64 (td, J=11.6, 3.20 Hz, 1H), 3.40-3.30 (m, 1H), 3.29-3.19 (m, 1H), 3.05-2.93 (m, 1H), 2.67 (br d, J=13.6 Hz, 1H), 1.01 (d, J=6.40 Hz, 3H).

### Intermediate 63: (2S,4S)-4-((6-Chloro-4-((S)-9-methyl-2,5-dioxa-8-azaspiro[3.5]nonan-8-yl)pyridazin-3-yl)oxy)-1-(2-(difluoromethyl)-7-fluoro-6-methylbenzofuro[3,2-d]pyrimidin-4-yl)pyrrolidine-2-carboxylic acid

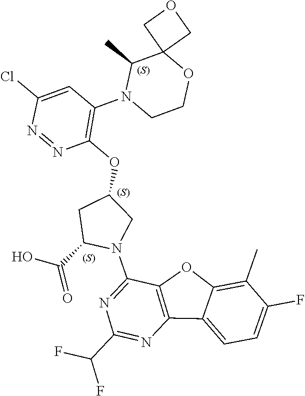

The title compound was prepared in a manner to Intermediate 62 using (S)-8-(3,6-dichloropyridazin-4-yl)-9-methyl-2,5-dioxa-8-azaspiro[3.5]nonane (Intermediate 30 product from Step B) and (2S,4S)-1-(2-(difluoromethyl)-7-fluoro-6-methylbenzofuro[3,2-d]pyrimidin-4-yl)-4-hydroxypyrrolidine-2-carboxylic acid (Intermediate 32). LCMS (ESI): mass calcd for C₂₈H₂₆ClF₃N₆O₆ 634.2, r/z found, 635.4 [M+H]⁺. ¹H NMR (600 MHz, dioxane) δ 9.03 (dd, J=8.6, 5.2 Hz, 1H), 8.40 (t, J=9.2 Hz, 1H), 8.01 (s, 1H), 7.83 (t, J=54.7 Hz, 1H), 6.81 (s, 1H), 5.66 (s, 1H), 5.58 (d, J=6.6 Hz, 1H), 5.51 (s, 1H), 5.39 (s, 1H), 5.17 (d, J=6.8 Hz, 1H), 4.88-4.76 (m, 1H), 4.73-4.60 (m, 1H), 4.39 (s, 1H), 4.28 (t, J=12.1 Hz, 1H), 4.05 (s, 1H), 3.73 (s, 1H), 3.51 (s, 3H), 2.03 (d, J=6.6 Hz, 3H).

### Example 1: (2S,4S)-1-(2-(Difluoromethyl)benzofuro[3,2-d]pyrimidin-4-yl)-4-((5-(3-methoxyprop-1-yn-1-yl)-3-((S)-9-methyl-2,5-dioxa-8-azaspiro[3.5]nonan-8-yl)pyridin-2-yl)oxy)pyrrolidine-2-carboxylic acid

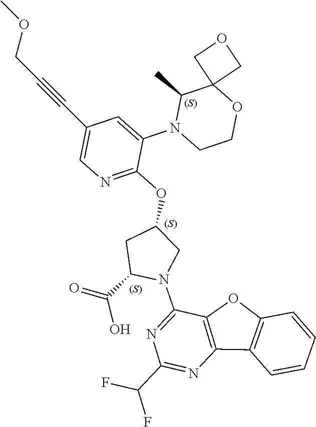

(2S,4S)-4-((5-Bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12, 50.0 mg, 0.0720 mmol), MeCN (1.0 mL), a stir bar, 3-methoxyprop-1-yne (10.0 mg, 0.143 mmol), Cs₂CO₃ (65.0 mg, 0.199 mmol), Xphos Pd G3 (7.5 mg, 8.9 μmol) and Xphos (5.0 mg, 0.10 mmol) were added to nitrogen-purged 8 mL vial. The black suspension was stirred at 70° C. for 2 h then cooled down to rt, filtered, and the filter cake was rinsed with EtOAc (20 mL). The filtrate was concentrated in vacuo to give a brown oil, which was subjected to HPLC (Welch Xtimate C18 5 μm, 150 mm×30 mm, 7 min gradient (57-77% ACN/H₂O (with 0.02% FA)) at 25 mL/min) to yield (2S,4S)-1-(2-(difluoromethyl)benzofuro[3,2-d]pyrimidin-4-yl)-4-((5-(3-methoxyprop-1-yn-1-yl)-3-((S)-9-methyl-2,5-dioxa-8-azaspiro[3.5]nonan-8-yl)pyridin-2-yl)oxy)pyrrolidine-2-carboxylic acid as an off-white powder (18.2 mg, 38%). LCMS (ESI): mass calcd for C₃₂H₃₁F₂N₅O₇, 635.2; m/z found, 636.2 [M+H]⁺ ¹H NMR (400 MHz, CDCl₃) δ 8.25 (d, J=8.0 Hz, 1H), 7.97 (d, J=1.6 Hz, 1H), 7.68-7.57 (m, 2H), 7.50-7.44 (m, 1H), 7.13 (d, J=2.0 Hz, 1H), 6.84-6.47 (m, 1H), 6.28 (s, 1H), 5.64 (s, 1H), 4.95 (s, 1H), 4.78 (s, 1H), 4.69-4.42 (m, 4H), 4.35 (s, 3H), 3.96-3.82 (m, 1H), 3.71-3.56 (m, 1H), 3.49 (s, 3H), 3.35-3.19 (m, 1H), 2.96-2.86 (m, 1H), 2.78 (s, 1H), 2.65-2.53 (m, 1H), 0.91 (d, J=6.8 Hz, 3H).

### Examples 2-8, in Table 8 were prepared in a manner analogous to Example 73, employing Sonogashira coupling conditions with (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (intermediate 12) and the corresponding alkyne

TABLE 8

| Ex # | Structure | Alkyne | MS |
|---|---|---|---|
| 2 | 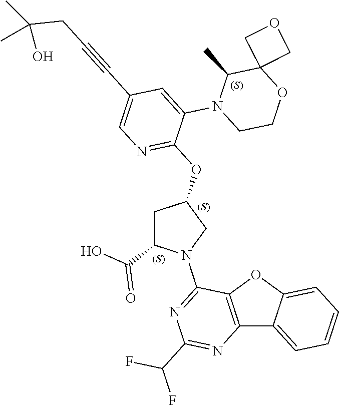 | 2-methylpent-4- yn-2-ol | mass calcd for C₃₄H₃₅F₂N₅O₇ 663.30 m/z found 664.30 [M + H]⁺ |
| | (2S,4S)-1-(2-(Difluoromethyl)benzofuro[3,2- | | |
| | d]pyrimidin-4-yl)-4-((5-(4-hydroxy-4-methylpent-1- | | |
| | yn-1-yl)-3-((S)-9-methyl-2,5-dioxa-8- | | |
| | azaspiro[3.5]nonan-8-yl)pyridin-2-yl)oxy)pyrrolidine- | | |
| | 2-carboxylic acid; | | |
| 3 | 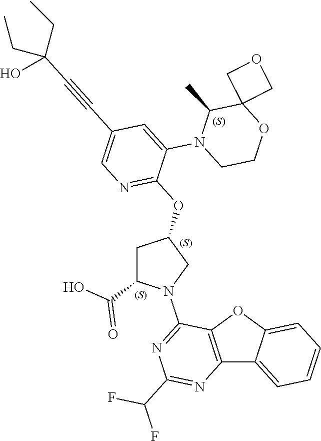 | 3-ethylpent-1- yn-3-ol | mass calcd for C₃₅H₃₇F₂N₅O₇ 677.30 m/z found 678.60 [M + H]⁺ |
| | (2S,4S)-1-(2-(Difluoromethyl)benzofuro[3,2- | | |
| | d]pyrimidin-4-yl)-4-((5-(3-ethyl-3-hydroxypent-1-yn- | | |
| | 1-yl)-3-((S)-9-methyl-2,5-dioxa-8- | | |
| | azaspiro[3.5]nonan-8-yl)pyridin-2-yl)oxy)pyrrolidine- | | |
| | 2-carboxylic acid; | | |
| 4 | 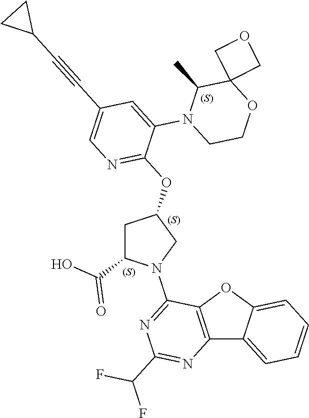 | Ethynyl cyclopropane | mass calcd for C₃₃H₃₁F₂N₅O₆ 631.20 m/z found 632.50 [M + H]⁺ |
| | (2S,4S)-4-((5-(Cyclopropylethynyl)-3-((S)-9-methyl- | | |
| | 2,5-dioxa-8-azaspiro[3.5]nonan-8-yl)pyridin-2- | | |
| | yl)oxy)-1-(2-(difluoromethyl)benzofuro[3,2- | | |
| | d]pyrimidin-4-yl)pyrrolidine-2-carboxylic acid; | | |
| 5 | 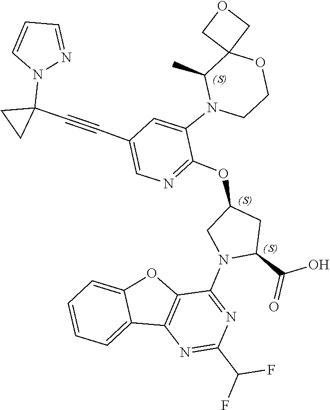 | 1-(1- ethynylcyclopropyl)- 1H-pyrazole | mass calcd for C₃₆H₃₃F₂N₇O₆ 697.20 m/z found 698.80 [M + H]⁺ |
| | (2S,4S)-4-((5-((1-(1H-Pyrazol-1- | | |
| | yl)cyclopropyl)ethynyl)-3-((S)-9-methyl-2,5-dioxa-8- | | |
| | azaspiro[3.5]nonan-8-yl)pyridin-2-yl)oxy)-1-(2- | | |
| | (difluoromethyl)benzofuro[3,2-d]pyrimidin-4- | | |
| | yl)pyrrolidine-2-carboxylic acid; | | |
| 6 | 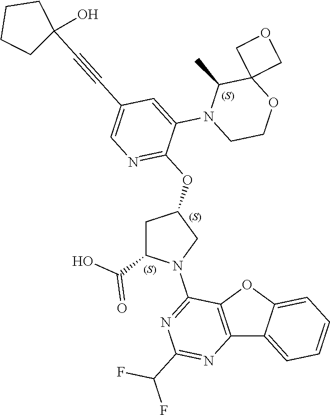 | 1- ethynylcyclopentan- 1-ol | mass calcd for C₃₅H₃₅F₂N₅O₇ 675.30 m/z found 676.60 [M + H]⁺ |
| | (2S,4S)-1-(2-(Difluoromethyl)benzofuro[3,2- | | |
| | d]pyrimidin-4-yl)-4-((5-((1- | | |
| | hydroxycyclopentyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 7 | 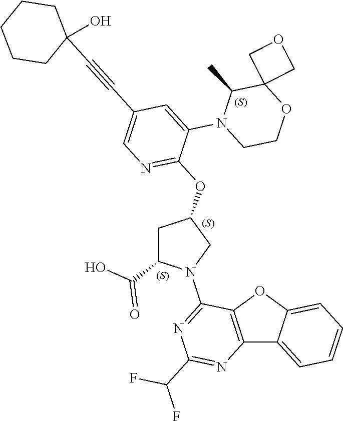 | 1- ethynylcyclohexan- 1-ol | mass calcd for C₃₆H₃₇F₂N₅O₇ 689.30 m/z found 690.30 [M + H]⁺ |
| | (2S,4S)-1-(2-(Difluoromethyl)benzofuro[3,2- | | |
| | d]pyrimidin-4-yl)-4-((5-((1- | | |
| | hydroxycyclohexyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 8 | 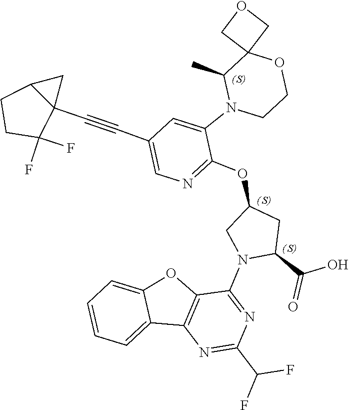 | 1-ethynyl-2,2- difluorobicyclo [3.1.0]hexane | mass calcd for C₃₆H₃₃F₄N₅O₆ 707.20 m/z found 708.30 [M + H]⁺ |
| | (2S,4S)-4-((5-((2,2-Difluorobicyclo[3.1.0]hexan-1- | | |
| | yl)ethynyl)-3-((S)-9-methyl-2,5-dioxa-8- | | |
| | azaspiro[3.5]nonan-8-yl)pyridin-2-yl)oxy)-1-(2- | | |
| | (difluoromethyl)benzofuro[3,2-d]pyrimidin-4- | | |
| | yl)pyrrolidine-2-carboxylic acid; | | |

### Example 9: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(phenylethynyl)pyridin-2-yl)oxy)pyrrolidine-2-carboxylic acid

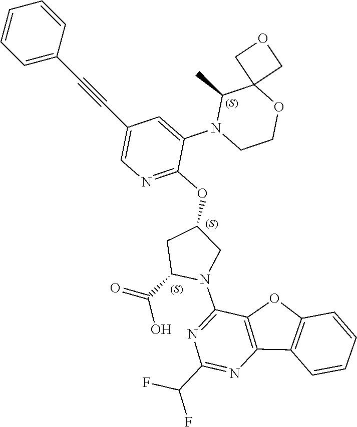

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and ethynylbenzene instead of 3-methoxyprop-1-yne. LCMS (ESI): mass calcd for C₃₆H₃₁F₂N₅O₆, 667.2; m/z found, 668.3 [M+H]⁺. ¹H NMR (400 MHz, CDCl₃) δ 8.24 (d, J=7.6 Hz, 1H), 8.04 (d, J=1.2 Hz, 1H), 7.68-7.59 (m, 2H), 7.57-7.51 (m, 2H), 7.50-7.43 (m, 1H), 7.42-7.33 (m, 3H), 7.22-7.14 (m, 1H), 6.64 (t, J=55.6 Hz, 1H), 6.36-6.25 (m, 1H), 5.86-5.23 (m, 1H), 5.09-4.75 (m, 2H), 4.75-4.37 (m, 4H), 4.36-3.93 (m, 1H), 3.93-3.81 (m, 1H), 3.70-3.58 (m, 1H), 3.37-3.24 (m, 1H), 2.97-2.88 (m, 1H), 2.88-2.72 (m, 1H), 2.70-2.63 (m, 1H), 0.93 (d, J=6.8 Hz, 3H).

### Examples 11-34, in Table 9 were prepared in a manner analogous to Example 73, employing Sonogashira coupling conditions with (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and the corresponding alkyne

TABLE 9

| Ex # | Structure | Alkyne | MS |
|---|---|---|---|
| 11 | 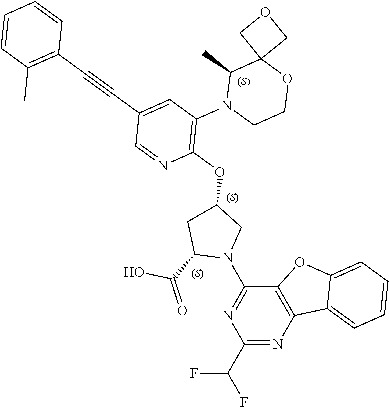 | 1-ethynyl-2- methylbenzene | mass calcd for C₃₇H₃₃F₂N₅O₆ 681.2 m/z found 682.3 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(o- | | |
| | tolylethynyl)pyridin-2-yl)oxy)pyrrolidine-2- | | |
| | carboxylic acid; | | |
| 12 | 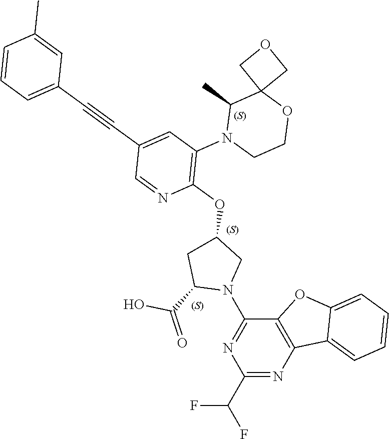 | 1-ethynyl-3- methylbenzene | mass calcd for C₃₇H₃₃F₂N₅O₆ 681.2 m/z found 682.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(m- | | |
| | tolylethynyl)pyridin-2-yl)oxy)pyrrolidine-2- | | |
| | carboxylic acid; | | |
| 13 | 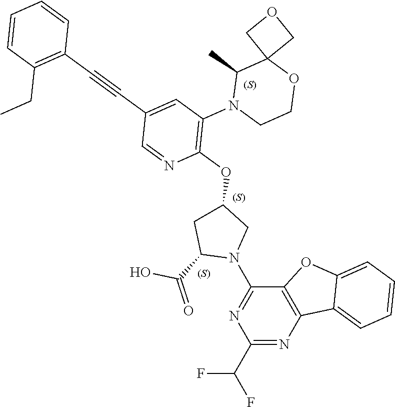 | 1-ethyl-2- ethynylbenzene | mass calcd for C₃₈H₃₅F₂N₅O₆ 695.3 m/z found 696.40 [M + H]⁺ |
| | (2S,4S)-1-(2-(Difluoromethyl)benzofuro[3,2- | | |
| | d]pyrimidin-4-yl)-4-((5-((2- | | |
| | ethylphenyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 14 | 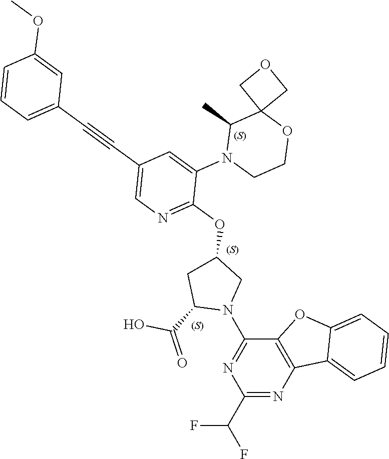 | 1-ethynyl-3- methoxybenzene | mass calcd for C₃₇H₃₃F₂N₅O₇ 697.20 m/z found 698.50 [M + H]⁺ |
| | (2S,4S)-1-(2-(Difluoromethyl)benzofuro[3,2- | | |
| | d]pyrimidin-4-yl)-4-((5-((3- | | |
| | methoxyphenyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 15 | 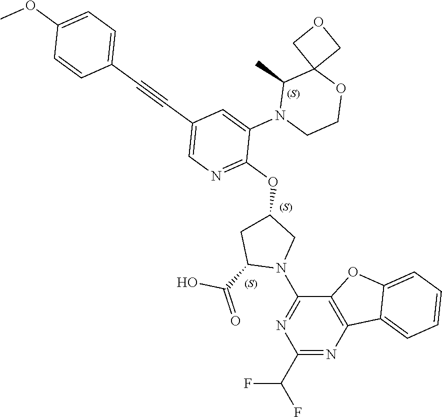 | 1-ethynyl-4- methoxybenzene | mass calcd for C₃₇H₃₃F₂N₅O₇ 697.20 m/z found 698.60 [M + H]⁺ |
| | (2S,4S)-1-(2-(Difluoromethyl)benzofuro[3,2- | | |
| | d]pyrimidin-4-yl)-4-((5-((4- | | |
| | methoxyphenyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 16 | 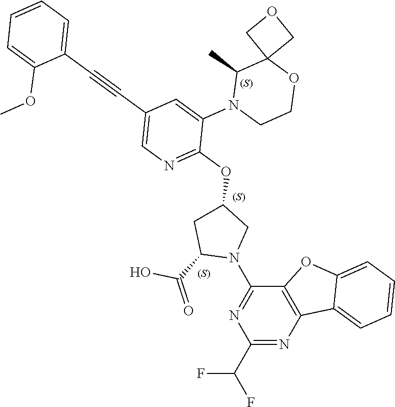 | 1-ethynyl-2- methoxybenzene | mass calcd for C₃₇H₃₃F₂N₅O₇ 697.20 m/z found 698.60 [M + H]⁺ |
| | (2S,4S)-1-(2-(Difluoromethyl)benzofuro[3,2- | | |
| | d]pyrimidin-4-yl)-4-((5-((2- | | |
| | methoxyphenyl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 17 | 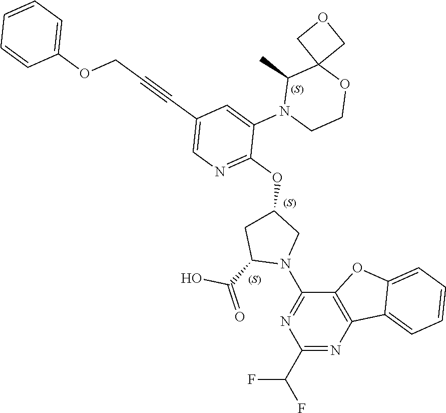 | (prop-2-yn-1- yloxy)benzene | mass calcd for C₃₇H₃₃F₂N₅O₇ 697.20 m/z found 698.60 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3-phenoxyprop- | | |
| | 1-yn-1-yl)pyridin-2-yl)oxy)pyrrolidine-2- | | |
| | carboxylic acid; | | |
| 18 | 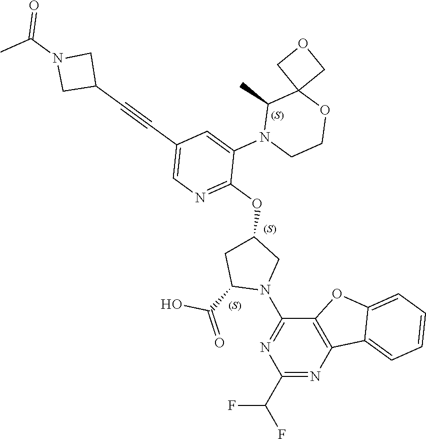 | 1-(3-ethynylazetidin-1- yl)ethan-1-one | mass calcd for C₃₅H₃₄F₂N₆O₇ 688.20 m/z found 689.40 [M + H]⁺ |
| | (2S,4S)-4-((5-((1-Acetylazetidin-3-yl)ethynyl)-3- | | |
| | ((S)-9-methyl-2,5-dioxa-8-azaspiro[3.5]nonan- | | |
| | 8-yl)pyridin-2-yl)oxy)-1-(2- | | |
| | (difluoromethyl)benzofuro[3,2-d]pyrimidin-4- | | |
| | yl)pyrrolidine-2-carboxylic acid; | | |
| 19 | 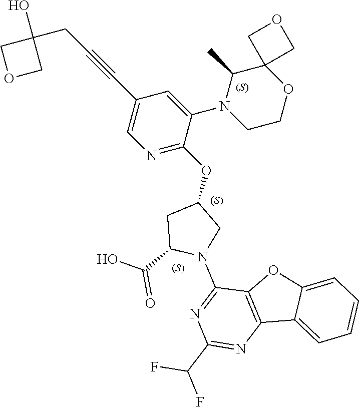 | 3-(prop-2-yn-1- yl)oxetan-3-ol | mass calcd for C₃₄H₃₃F₂N₅O₈ 677.20 m/z found 678.30 [M + H]⁺ |
| | (2S,4S)-1-(2-(Difluoromethyl)benzofuro[3,2- | | |
| | d]pyrimidin-4-yl)-4-((5-(3-(3-hydroxyoxetan-3- | | |
| | yl)prop-1-yn-1-yl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 20 | 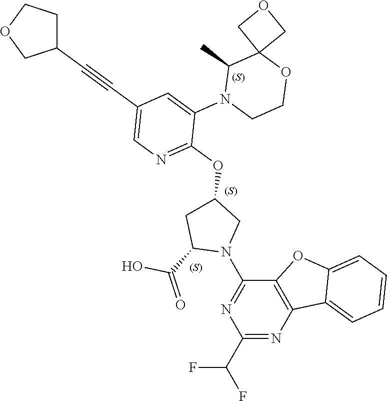 | 3- ethynyltetrahydrofuran | mass calcd for C₃₄H₃₃F₂N₅O₇ 661.20 m/z found 662.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5- | | |
| | ((tetrahydrofuran-3-yl)ethynyl)pyridin-2- | | |
| | yl)oxy)pyrrolidine-2-carboxylic acid; | | |
| 21 | 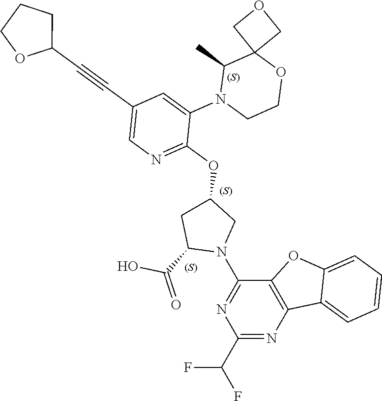 | 2- ethynyltetrahydrofuran | mass calcd for C₃₄H₃₃F₂N₅O₇ 661.20 m/z found 662.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5- | | |
| | ((tetrahydrofuran-2-yl)ethynyl)pyridin-2- | | |
| | yl)oxy)pyrrolidine-2-carboxylic acid; | | |
| 22 | 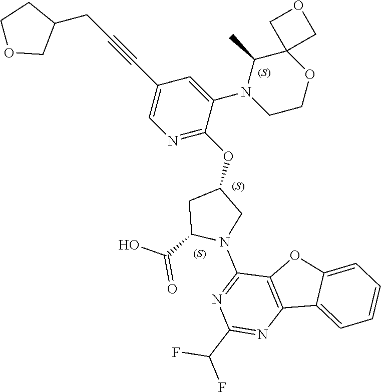 | 3-(prop-2-yn-1- yl)tetrahydrofuran | mass calcd for C₃₅H₃₅F₂N₅O₇ 675.30 m/z found 676.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3- | | |
| | (tetrahydrofuran-3-yl)prop-1-yn-1-yl)pyridin-2- | | |
| | yl)oxy)pyrrolidine-2-carboxylic acid; | | |
| 23 | 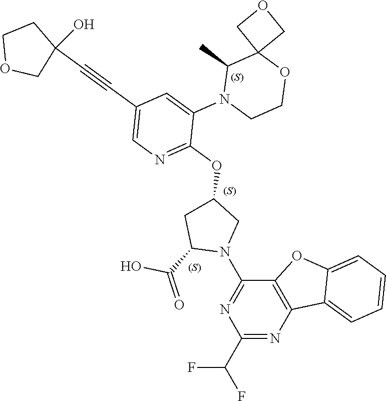 | 3- ethynyltetrahydrofuran- 3-ol | mass calcd for C₃₄H₃₃F₂N₅O₈ 677.20 m/z found 678.50 [M + H]⁺ |
| | (2S,4S)-1-(2-(Difluoromethyl)benzofuro[3,2- | | |
| | d]pyrimidin-4-yl)-4-((5-((3- | | |
| | hydroxytetrahydrofuran-3-yl)ethynyl)-3-((S)-9- | | |
| | methyl-2,5-dioxa-8-azaspiro[3.5]nonan-8- | | |
| | yl)pyridin-2-yl)oxy)pyrrolidine-2-carboxylic | | |
| | acid; | | |
| 24 | 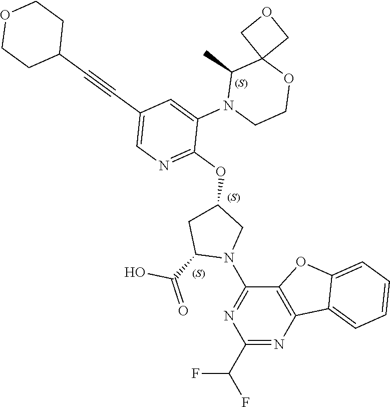 | 4-ethynyltetrahydro- 2H-pyran | mass calcd for C₃₅H₃₅F₂N₅O₇ 675.30 m/z found 676.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-((tetrahydro-2H- | | |
| | pyran-4-yl)ethynyl)pyridin-2-yl)oxy)pyrrolidine- | | |
| | 2-carboxylic acid; | | |
| 25 | 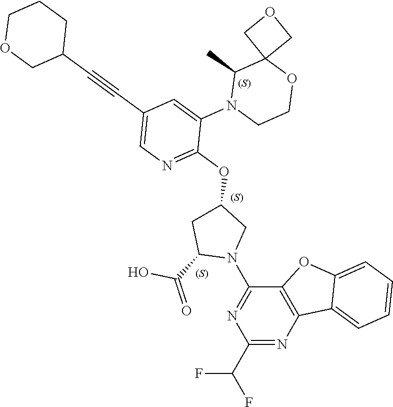 | 3-ethynyltetrahydro- 2H-pyran | mass calcd for C₃₅H₃₅F₂N₅O₇ 675.30 m/z found 676.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-((tetrahydro-2H- | | |
| | pyran-3-yl)ethynyl)pyridin-2-yl)oxy)pyrrolidine- | | |
| | 2-carboxylic acid; | | |
| 26 | 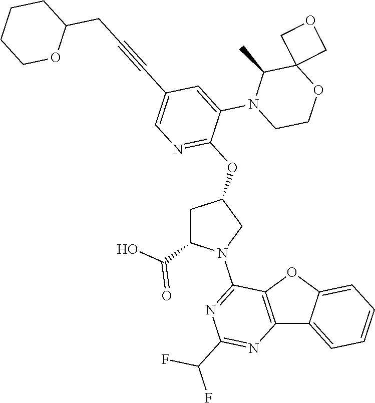 [SMILES: C[C@@H]1N(c2cc(C#CCC3CCCCO3)cnc2O[C@H]2C[C@@H](C(=O)O)N(c3nc(C(F)F)nc4c3oc3ccccc34)C2)CCOC12COC2] | 2-(prop-2-yn-1-yl)- tetrahydro-2H-pyran | mass calcd for C₃₆H₃₇F₂N₅O₇ 689.30 m/z found 690.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3-(tetrahydro- | | |
| | 2H-pyran-2-yl)prop-1-yn-1-yl)pyridin-2- | | |
| | yl)oxy)pyrrolidine-2-carboxylic acid; | | |
| 27 | 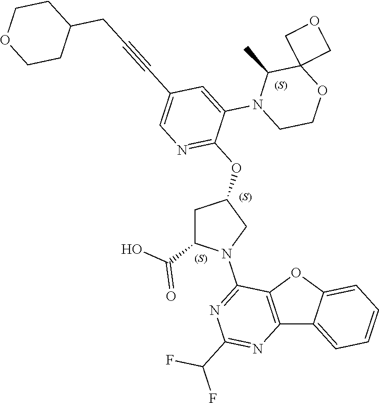 | 4-(prop-2-yn-1- yl)tetrahydro-2H-pyran | mass calcd for C₃₆H₃₇F₂N₅O₇ 689.30 m/z found 690.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3-(tetrahydro- | | |
| | 2H-pyran-4-yl)prop-1-yn-1-yl)pyridin-2- | | |
| | yl)oxy)pyrrolidine-2-carboxylic acid; | | |
| 28 | 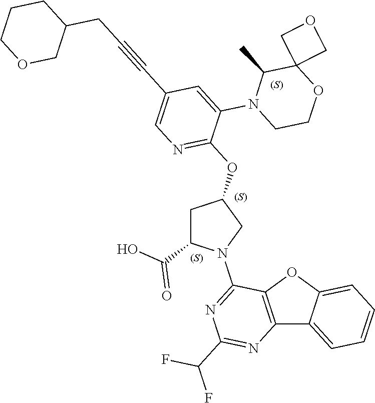 | 3-(prop-2-yn-1- yl)tetrahydro-2H-pyran | mass calcd for C₃₆H₃₇F₂N₅O₇ 689.30 m/z found 690.60 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3-(tetrahydro- | | |
| | 2H-pyran-3-yl)prop-1-yn-1-yl)pyridin-2- | | |
| | yl)oxy)pyrrolidine-2-carboxylic acid; | | |
| 29 | 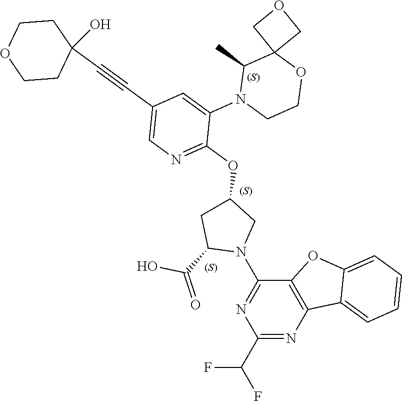 | 4-ethynyltetrahydro- 2H-pyran-4-ol | mass calcd for C₃₅H₃₅F₂N₅O₈ 691.20 m/z found 692.50 [M + H]⁺ |
| | (2S,4S)-1-(2-(Difluoromethyl)benzofuro[3,2- | | |
| | d]pyrimidin-4-yl)-4-((5-((4-hydroxytetrahydro- | | |
| | 2H-pyran-4-yl)ethynyl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |
| 30 | 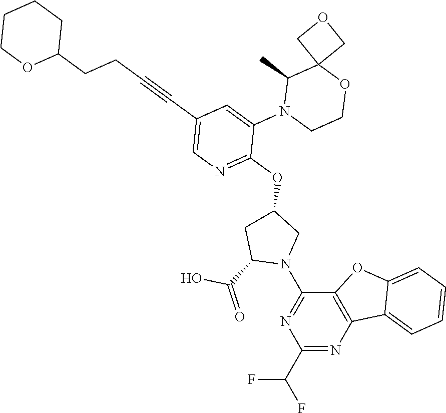 [SMILES: C[C@@H]1N(c2cc(C#CCCC3CCCCO3)cnc2O[C@H]2C[C@@H](C(=O)O)N(c3nc(C(F)F)nc4c3oc3ccccc34)C2)CCOC12COC2] | 2-(but-3-yn-1- yl)tetrahydro-2H-pyran | mass calcd for C₃₇H₃₉F₂N₅O₇ 703.30 m/z found 704.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(4-(tetrahydro- | | |
| | 2H-pyran-2-yl)but-1-yn-1-yl)pyridin-2- | | |
| | yl)oxy)pyrrolidine-2-carboxylic acid; | | |
| 31 | 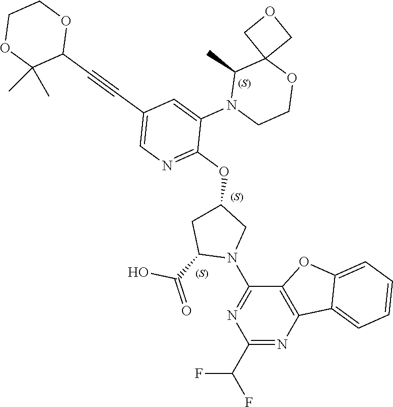 | 3-ethynyl-2,2- dimethyl-1,4-dioxane | mass calcd for C₃₆H₃₇F₂N₅O₈ 705.30 m/z found 706.40 [M + H]⁺ |
| | (2S,4S)-1-(2-(Difluoromethyl)benzofuro[3,2- | | |
| | d]pyrimidin-4-yl)-4-((5-((3,3-dimethyl-1,4- | | |
| | dioxan-2-yl)ethynyl)-3-((S)-9-methyl-2,5-dioxa- | | |
| | 8-azaspiro[3.5]nonan-8-yl)pyridin-2- | | |
| | yl)oxy)pyrrolidine-2-carboxylic acid; | | |
| 32 | 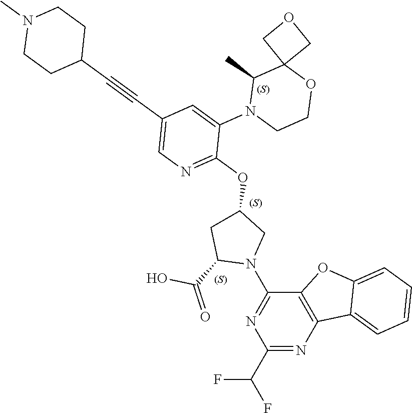 | 4-ethynyl-1- methylpiperidine | mass calcd for C₃₆H₃₈F₂N₆O₆ 688.30 m/z found 689.40 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-((1- | | |
| | methylpiperidin-4-yl)ethynyl)pyridin-2- | | |
| | yl)oxy)pyrrolidine-2-carboxylic acid; | | |
| 33 | 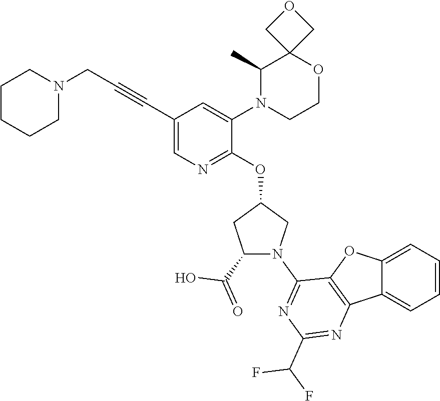 [SMILES: C[C@@H]1N(c2cc(C#CCN3CCCCC3)cnc2O[C@H]2C[C@@H](C(=O)O)N(c3nc(C(F)F)nc4c3oc3ccccc34)C2)CCOC12COC2] | 1-(prop-2-yn-1- yl)piperidine | mass calcd for C₃₆H₃₈F₂N₆O₆ 688.30 m/z found 689.50 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa- | | |
| | 8-azaspiro[3.5]nonan-8-yl)-5-(3-(piperidin-1- | | |
| | yl)prop-1-yn-1-yl)pyridin-2-yl)oxy)pyrrolidine- | | |
| | 2-carboxylic acid; | | |
| 34 | 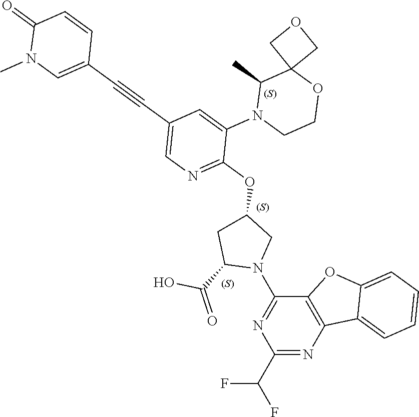 | 5-ethynyl-1- methylpyridin-2(1H)- one | mass calcd for C₃₆H₃₂F₂N₆O₇ 698.20 m/z found 699.40 [M + H]⁺ |
| | (2S,4S)-1-(2-(Difluoromethyl)benzofuro[3,2- | | |
| | d]pyrimidin-4-yl)-4-((5-((1-methyl-6-oxo-1,6- | | |
| | dihydropyridin-3-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |

### Example 35: (2S,4S)-1-(2-(Difluoromethyl)benzofuro[3,2-d]pyrimidin-4-yl)-4-((5-((1-methyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid

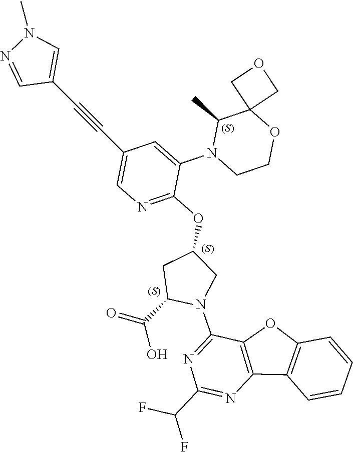

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 4-ethynyl-1-methyl-1H-pyrazole instead of 3-methoxyprop-1-yne, and the reaction mixture was heated to 90° C. for 2 h instead of 70° C. for 2 h. LCMS (ESI): mass calcd for C₃₁H₃₁F₂N₇O₆, 671.2; m/z found, 672.3 [M+H]⁺. ¹H NMR (400 MHz, CDCl₃) δ 8.24 (d, J=8.0 Hz, 1H), 7.98 (d, J=1.6 Hz, 1H), 7.66 (s, 1H), 7.64-7.58 (m, 2H), 7.57 (s, 1H), 7.50-7.43 (m, 1H), 7.15 (d, J=2.0 Hz, 1H), 6.79-6.49 (m, 1H), 6.29 (s, 1H), 5.78-5.29 (m, 1H), 4.95 (s, 1H), 4.77 (s, 2H), 4.65-4.40 (m, 4H), 3.93 (s, 3H), 3.90-3.85 (m, 1H), 3.68-3.56 (m, 1H), 3.35-3.23 (m, 1H), 2.95-2.86 (m, 1H), 2.79 (s, 1H), 2.63 (d, J=11.2 Hz, 1H), 0.91 (d, J=6.4 Hz, 3H).

### Example 36: (2S,4S)-1-(2-(Difluoromethyl)benzofuro[3,2-d]pyrimidin-4-yl)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid

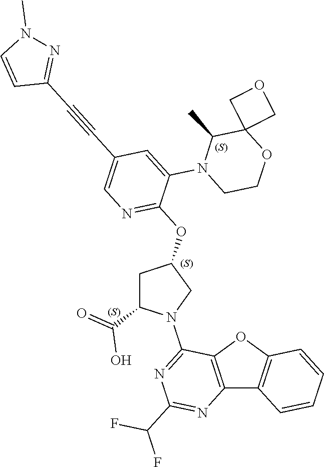

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 3-ethynyl-1-methyl-1H-pyrazole instead of 3-methoxyprop-1-yne, and the reaction mixture was heated to 90° C. for 2 h instead of 70° C. for 2 h. LCMS (ESI): mass calcd for C₃₄H₃₁F₂N₇O₆, 671.2; m/z found, 672.2 [M+H]⁺. ¹H NMR (400 MHz, CDCl₃) δ 8.24 (d, J=7.6 Hz, 1H), 8.04 (d, J=1.6 Hz, 1H), 7.68-7.56 (m, 2H), 7.50-7.41 (m, 1H), 7.37 (d, J=2.4 Hz, 1H), 7.22 (d, J=1.6 Hz, 1H), 6.64 (t, J=55.2 Hz, 1H), 6.48-6.44 (m, 1H), 6.34-6.25 (m, 1H), 5.72-5.40 (m, 1H), 5.00-4.92 (m, 1H), 4.84-4.72 (m, 1H), 4.67-4.40 (m, 4H), 3.95 (s, 3H), 3.91-3.83 (m, 1H), 3.69-3.57 (m, 1H), 3.37-3.19 (m, 1H), 2.94-2.86 (m, 1H), 2.83-2.73 (m, 1H), 2.68-2.57 (m, 2H), 0.90 (d, J=6.4 Hz, 3H).

### Example 37: (2S,4S)-1-(2-(Difluoromethyl)-7-fluorobenzofuro[3,2-d]pyrimidin-4-yl)-4-((5-((1-methyl-1H-pyrazol-3-yl)ethynyl)-3-((R 1-9-methyl-2,5-dioxa-8-azaspiro[3.5]nonan-8-yl)pyridin-2-yl)oxy)pyrrolidine-2-carboxylic acid

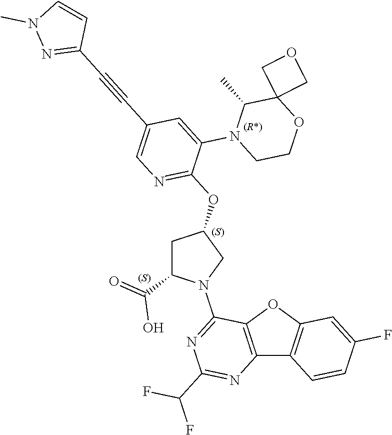

(2S,4S)-4-((5-((1-Methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid (Intermediate 15, 70.0 mg crude as TFA salt), a stir bar, DMSO (1.0 mL), 4-chloro-2-(difluoromethyl)-7-fluorobenzofuro[3,2-d]pyrimidine (Intermediate 2, 34.0 mg, 0.122 mmol) and DIEA (47.0 mg, 0.364 mmol) were added to an 8 mL vial. The yellow suspension was stirred at 110° C. for 1 h, cooled down to rt, and directly subjected to HPLC (Welch Xtimate C18 5 μm, 150 mm×30 mm; 7 min gradient (55-85% ACN/H₂O (with 0.02% FA)) at 25 mL/min) to yield the title compound, (2S,4S)-1-(2-(difluoromethyl)-7-fluorobenzofuro[3,2-d]pyrimidin-4-yl)-4-((5-((1-methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid as an off-white powder (25.7 mg, 30%). LCMS (ESI): mass calcd for C₃₄H₃₀F₃N₇O₆, 689.2; m/z found, 690.2 [M+H]⁺. ¹H NMR (400 MHz, CDCl₃) δ 8.23-8.16 (m, 1H), 8.03 (d, J=1.6 Hz, 1H), 7.37 (d, J=2.0 Hz, 1H), 7.32 (dd, J=2.0, 8.4 Hz, 1H), 7.25-7.18 (m, 2H), 6.77-6.48 (m, 1H), 6.47 (d, J=2.4 Hz, 1H), 6.31 (t, J=6.0 Hz, 1H), 5.72-5.34 (m, 1H), 4.97 (d, J=6.4 Hz, 1H), 4.87-4.57 (m, 3H), 4.56-4.41 (m, 3H), 3.95 (s, 3H), 3.88 (dd, J=3.2, 11.6 Hz, 1H), 3.67-3.58 (m, 1H), 3.32-3.22 (m, 1H), 2.96-2.67 (m, 2H), 2.63 (s, 1H), 0.91-0.91 (m, 1H), 0.91 (d, J=6.8 Hz, 2H).

### Example 38: (2S,4S)-1-(2-(Difluoromethyl)-7-fluorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

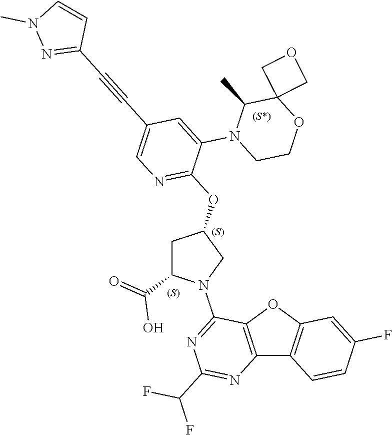

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-chloro-2-(difluoromethyl)-7-fluorobenzofuro[3,2-d]pyrimidine (Intermediate 2). LCMS (ESI): mass calcd for C₃₄H₃₀F₃N₇O₆, 689.2; m/z found, 690.2 [M+H]⁺. ¹H NMR (400 MHz, CDCl₃) δ 11.85-10.73 (m, 1H), 8.29-8.14 (m, 1H), 8.09 (d, J=18.0 Hz, 1H), 7.50-7.31 (m, 2H), 7.21 (s, 2H), 6.82-6.50 (m, 1H), 6.49-6.43 (m, 1H), 5.93-5.69 (m, 1H), 5.64-5.40 (m, 1H), 5.07-4.82 (m, 2H), 4.82-4.71 (m, 1H), 4.68-4.49 (m, 2H), 4.48-4.37 (m, 1H), 4.36-4.22 (m, 1H), 3.95 (s, 3H), 3.94-3.65 (m, 2H), 3.46-3.29 (m, 1H), 3.06-2.75 (m, 2H), 2.65-2.48 (m, 1H), 0.80 (d, J=6.4 Hz, 3H).

### Example 39: (2S,4S)-1-(2-(Difluoromethyl)-9-fluorobenzofuro[3,2-d]pyrimidin-4-yl)-4-((5-((1-methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid

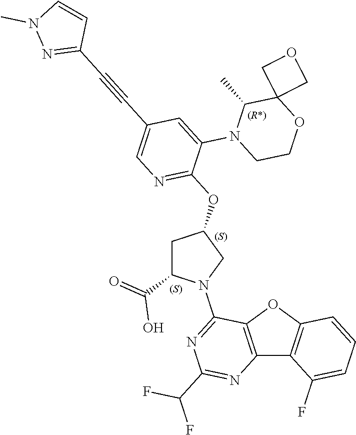

The title compound was prepared in a manner analogous to Example 37, using 4-chloro-2-(difluoromethyl)-9-fluorobenzofuro[3,2-d]pyrimidine (Intermediate 4) instead of 4-chloro-2-(difluoromethyl)-7-fluorobenzofuro[3,2-d]pyrimidine (Intermediate 2) and (2S,4S)-4-((5-((1-methyl-1H-pyrazol-3-yl)ethynyl)-3-((R)-9-methyl-2,5-dioxa-8-azaspiro[3.5]nonan-8-yl)pyridin-2-yl)oxy)pyrrolidine-2-carboxylic acid (Intermediate 14) instead of (2S,4S)-4-((5-((1-Methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate (Intermediate 15),and stirred at 100° C. for 30 min instead of 110° C. for 1 h. LCMS (ESI): mass calcd for C₃₄H₃₀F₃N₇O₆, 689.2; m/z found, 690.2 [M+H]⁺. ¹H NMR (400 MHz, CDCl₃) δ 8.04 (d, J=1.6 Hz, 1H), 7.62-7.52 (m, 1H), 7.44-7.39 (m, 1H), 7.37 (d, J=2.0 Hz, 1H), 7.22 (d, J=1.6 Hz, 1H), 7.15 (t, J=8.4 Hz, 1H), 6.66 (t, J=55.2 Hz, 1H), 6.47 (d, J=2.4 Hz, 1H), 6.34-6.29 (m, 1H), 5.67-5.43 (m, 1H), 4.99-4.94 (m, 1H), 4.83-4.55 (m, 3H), 4.54-4.45 (m, 2H), 3.95 (s, 3H), 3.91-3.85 (m, 1H), 3.66-3.58 (m, 1H), 3.33-3.20 (m, 1H), 2.94-2.65 (m, 3H), 2.64-2.58 (m, 1H), 0.91 (d, J=6.8 Hz, 3H).

### Example 40: (2S,4S)-1-(2-(Difluoromethyl)-9-fluorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

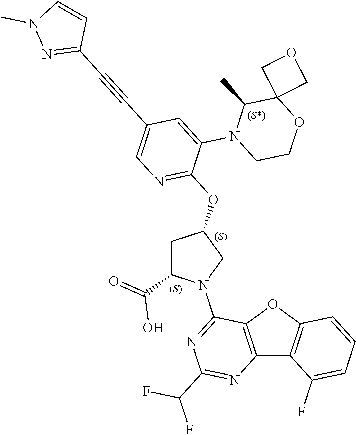

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-chloro-2-(difluoromethyl)-9-fluorobenzofuro[3,2-d]pyrimidine (Intermediate 4) instead of 4-chloro-2-(difluoromethyl)-7-fluorobenzofuro[3,2-d]pyrimidine (Intermediate 2). LCMS (ESI): mass calcd for C₃₄H₃₀F₃N₇O₆, 689.2; m/z found, 690.3 [M+H]⁺. ¹H NMR (400 MHz, CDCl₃) δ 8.13-8.05 (m, 1H), 7.61-7.52 (m, 1H), 7.51-7.39 (m, 1H), 7.37 (s, 1H), 7.22-7.18 (m, 1H), 7.16-7.09 (m, 1H), 6.83-6.52 (m, 1H), 6.47 (d, J=2.0 Hz, 1H), 5.88-5.72 (m, 1H), 5.60-5.43 (m, 1H), 4.98-4.91 (m, 1H), 4.91-4.72 (m, 3H), 4.58 (d, J=7.2 Hz, 1H), 4.57-4.42 (m, 1H), 4.41-4.24 (m, 1H), 3.95 (s, 3H), 3.94-3.88 (m, 1H), 3.74-3.64 (m, 1H), 3.38-3.29 (m, 1H), 2.99-2.91 (m, 1H), 2.85-2.77 (m, 1H), 2.56-2.50 (m, 1H), 0.81 (d, J=6.8 Hz, 3H).

### Example 41: (2S,4S)-1-(2-(Difluoromethyl)-6-methylbenzofuro[3,2-d]pyrimidin-4-yl)-4-((5-((1-methyl-1H-pyrazol-3-yl)ethynyl)-3-((R*)-9-methyl-2,5-dioxa-8-azaspiro[3.5]nonan-8-yl)pyridin-2-yl)oxy)pyrrolidine-2-carboxylic acid

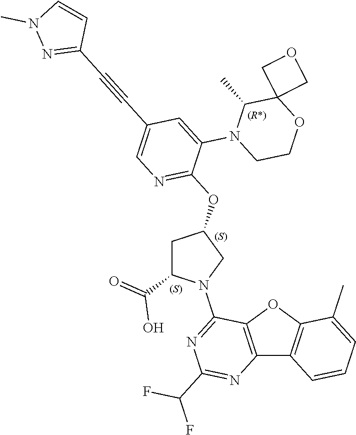

The title compound was prepared in a manner analogous to Example 37, using and 4-chloro-2-(difluoromethyl)-6-methylbenzofuro[3,2-d]pyrimidine (Intermediate 3) instead of 4-chloro-2-(difluoromethyl)-7-fluorobenzofuro[3,2-d]pyrimidine (Intermediate 2). LCMS (ESI): mass calcd for C₃₅H₃₅F₂N₇O₆, 685.2; m/z found, 686.3 [M+H]⁺. ¹H NMR (400 MHz, CDCl₃) δ 8.07-8.02 (m, 2H), 7.48-7.40 (m, 1H), 7.38-7.32 (m, 2H), 7.24-7.20 (m, 1H), 6.64 (t, J=55.2 Hz, 1H), 6.47 (d, J=2.0 Hz, 1H), 6.31-6.25 (m, 1H), 5.76-5.39 (m, 1H), 5.02-4.92 (m, 1H), 4.84-4.74 (m, 1H), 4.67-4.42 (m, 4H), 3.95 (s, 3H), 3.90-3.85 (m, 1H), 3.69-3.58 (m, 1H), 3.33-3.23 (m, 1H), 2.93-2.86 (m, 2H), 2.65-2.54 (m, 5H), 0.91 (d, J=6.4 Hz, 3H).

### Example 42: (2S,4S)-1-(2-(Difluoromethyl)-6-methylbenzofuro[3,2-d]pyrimidin-4-yl)-4-((5-((1-methyl-1H-pyrazol-3-yl)ethynyl)-3-(S 1-9-methyl-2,5-dioxa-8-azaspiro[3.5]nonan-8-yl)pyridin-2-yl)oxy)pyrrolidine-2-carboxylic acid

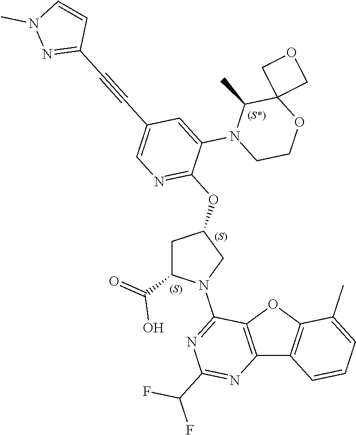

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-chloro-2-(difluoromethyl)-6-methylbenzofuro[3,2-d]pyrimidine (Intermediate 3) instead of 4-chloro-2-(difluoromethyl)-7-fluorobenzofuro[3,2-d]pyrimidine (Intermediate 2). LCMS (ESI): mass calcd for C₃₅H₃₅F₂N₇O₆, 685.2; m/z found, 686.3 [M+H]⁺. ¹H NMR (400 MHz, CDCl₃) δ 8.11-7.99 (m, 2H), 7.47-7.31 (m, 3H), 7.23-7.16 (m, 1H), 6.83-6.51 (m, 1H), 6.49-6.43 (m, 1H), 5.85-5.70 (m, 1H), 5.67-5.41 (m, 1H), 4.98-4.73 (m, 3H), 4.70-4.51 (m, 2H), 4.47-4.26 (m, 2H), 4.01-3.86 (m, 4H), 3.76-3.62 (m, 1H), 3.41-3.28 (m, 1H), 3.02-2.77 (m, 2H), 2.67-2.49 (m, 4H), 0.81 (d, J=6.4 Hz, 3H).

### Example 43: (2S,4S)-1-(2-(Difluoromethyl)-6-(trifluoromethyl)benzofuro[3,2-d]pyrimidin-4-yl)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid

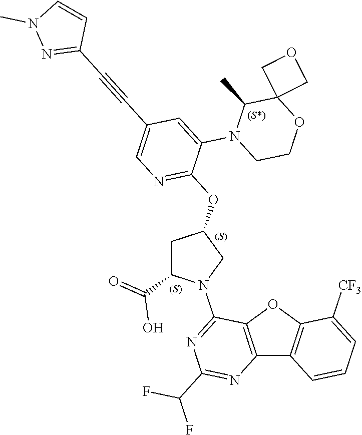

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15), and 4-chloro-2-(difluoromethyl)-6-(trifluoromethyl)benzofuro[3,2-d]pyrimidine (Intermediate 5) instead of 4-chloro-2-(difluoromethyl)-7-fluorobenzofuro[3,2-d]pyrimidine (Intermediate 2), and heating to 100° C. for 0.5 h instead of 110° C. for 1 h. LCMS (ESI): mass calcd for C₃₅H₃₀F₅N₇O₆, 739.2; m/z found, 740.3 [M+H]⁺. ¹H NMR (400 MHz, DMSO-d₆) δ 8.47 (d, J=7.6 Hz, 1H), 8.07 (d, J=8.0 Hz, 1H), 7.99 (d, J=1.2 Hz, 1H), 7.79-7.67 (m, 2H), 7.27 (d, J=1.6 Hz, 1H), 6.80 (t, J=54.4 Hz, 1H), 6.48 (d, J=2.0 Hz, 1H), 5.77-5.56 (m, 1H), 5.50-4.94 (m, 1H), 4.74-4.26 (m, 5H), 4.25-4.03 (m, 2H), 3.88 (s, 3H), 3.80-3.73 (m, 1H), 3.69-3.55 (m, 1H), 3.38-3.17 (m, 2H), 2.82-2.62 (m, 2H), 0.84 (d, J=6.4 Hz, 3H).

### Example 44: (2S,4S)-1-(2-Isopropylbenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

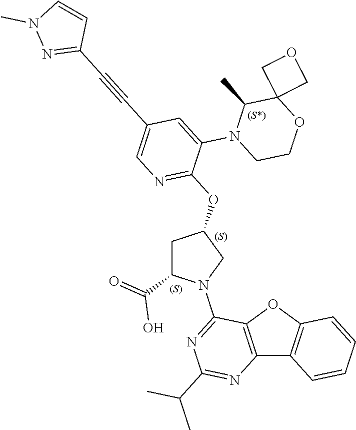

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-Chloro-2-isopropylbenzofuro[3,2-d]pyrimidine (Intermediate 7) instead of 4-chloro-2-(difluoromethyl)-7-fluorobenzofuro[3,2-d]pyrimidine (Intermediate 2), and heating to 100° C. for 0.5 h instead of 110° C. for 1 h. LCMS (ESI): mass calcd for C₃₆H₃₇N₇O₆, 663.3; r/z found, 664.3 [M+H]⁺. ¹H NMR (400 MHz, DMSO-d₆) δ 8.09 (d, J=8.0 Hz, 1H), 8.00 (d, J=1.6 Hz, 1H), 7.75-7.69 (m, 2H), 7.69-7.62 (m, 1H), 7.50-7.44 (m, 1H), 7.28 (d, J=2.0 Hz, 1H), 6.48 (d, J=2.0 Hz, 1H), 5.69 (s, 1H), 5.20-5.01 (m, 1H), 4.66 (d, J=6.4 Hz, 1H), 4.54 (d, J=6.4 Hz, 2H), 4.35 (d, J=6.8 Hz, 1H), 4.33-4.18 (m, 2H), 4.11 (d, J=6.8 Hz, 1H), 3.87 (s, 3H), 3.80-3.74 (m, 1H), 3.66 (dt, J=2.8, 11.2 Hz, 1H), 3.29 (dt, J=3.6, 11.6 Hz, 1H), 3.03-2.94 (m, 1H), 2.73 (d, J=11.6 Hz, 1H), 2.63-2.52 (m, 2H), 1.32 (dd, J=2.4, 6.8 Hz, 6H), 0.85 (d, J=6.8 Hz, 3H).

### Example 45: (2S,4S)-1-(2-(tert-Butyl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

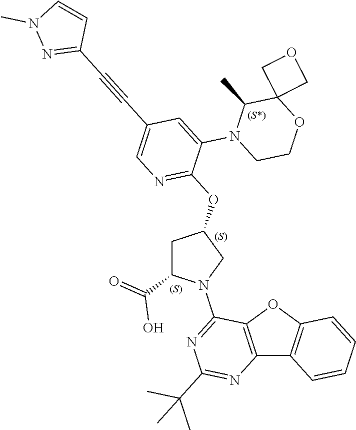

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 2-(Tert-butyl)-4-chlorobenzofuro[3,2-d]pyrimidine (Intermediate 8) instead of 4-chloro-2-(difluoromethyl)-7-fluorobenzofuro[3,2-d]pyrimidine (Intermediate 2), and heating to 100° C. for 0.5 h instead of 110° C. for 1 h. LCMS (ESI): mass calcd for C₃₇H₃₉N₇O₆, 677.3; r/z found, 678.3 [M+H]⁺. ¹H NMR (400 MHz, DMSO-d₆) δ 8.09 (d, J=7.6 Hz, 1H), 8.01 (d, J=2.0 Hz, 1H), 7.76-7.70 (m, 2H), 7.69-7.63 (m, 1H), 7.50-7.45 (m, 1H), 7.28 (d, J=2.0 Hz, 1H), 6.49 (d, J=2.0 Hz, 1H), 5.74-5.67 (m, 1H), 5.16-4.99 (m, 1H), 4.67 (d, J=6.4 Hz, 1H), 4.63-4.51 (m, 2H), 4.39-4.21 (m, 3H), 4.12 (d, J=6.8 Hz, 1H), 3.88 (s, 3H), 3.81-3.74 (m, 1H), 3.71-3.62 (m, 1H), 3.35-3.25 (m, 1H), 3.04-2.97 (m, 2H), 2.74 (d, J=11.6 Hz, 1H), 1.42 (s, 9H), 0.85 (d, J=6.8 Hz, 3H).

### Example 46: (2S,4S)-1-(2-Methoxybenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

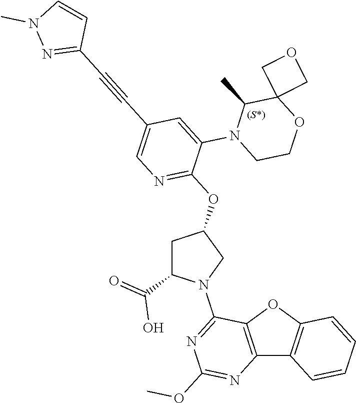

(2S,4S)-1-(2-chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid (Intermediate 16, 50.0 mg, 0.0713 mmol), a stir bar, MeOH (2.0 mL) and NaOMe (0.12 mL, 0.65 mmol, 5.4 M in MeOH) were added to an 8 mL vial. The yellow solution was stirred at 80° C. overnight and cooled down to rt to give a white suspension, which was filtered through a pad of diatomaceous earth, and the filter cake was rinsed with EtOAc (20 mL×2). The filtrate was concentrated to dryness in vacuo to give a white solid, which was subjected to prep-TLC (DCM/MeOH=10/1) and followed by HPLC (Welch Xtimate C18 5 μm, 150 mm×30 mm, 7 min gradient (45-75% ACN/H₂O (with 0.02% FA) at 25 mL/min) to afford the title compound, (2S,4S)-1-(2-methoxybenzofuro[3,2-d]pyrimidin-4-yl)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid as an off-white powder (1.5 mg, 3%). LCMS (ESI): mass calcd for C₃₄H₃₅N₇O₇, 651.2; m/z found, 652.3 [M+H]⁺. ¹H NMR (400 MHz, Acetonitrile-d₃) δ 8.33-8.20 (m, 1H), 7.97 (d, J=2.0 Hz, 1H), 7.71-7.61 (m, 2H), 7.50 (d, J=2.4 Hz, 1H), 7.49-7.44 (m, 1H), 7.23 (d, J=2.0 Hz, 1H), 6.42 (d, J=2.4 Hz, 1H), 5.80-5.74 (m, 1H), 4.77-4.70 (m, 1H), 4.67-4.43 (m, 2H), 4.43-4.25 (m, 2H), 4.25-4.13 (m, 1H), 4.08-3.99 (m, 3H), 3.86 (s, 3H), 3.81-3.74 (m, 1H), 3.70-3.58 (m, 1H), 3.33-3.23 (m, 1H), 2.98-2.76 (m, 2H), 2.69-2.56 (m, 1H), 1.30-1.23 (m, 2H), 0.82 (d, J=6.4 Hz, 3H).

### Example 47: (2S,4S)-1-(2-Ethoxybenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

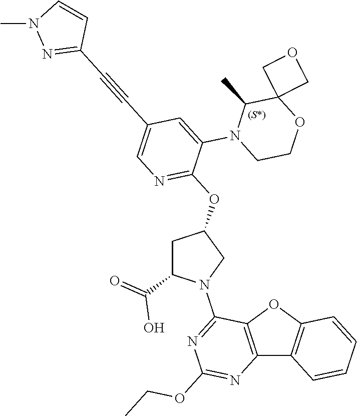

The title compound was prepared in a manner analogous to Example 46, using (2S,4S)-1-(2-chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid (Intermediate 16), EtOH instead of MeOH and NaOEt instead of NaOMe. LCMS (ESI): mass calcd for C₃₅H₃₅N₇O₇, 665.3; m/z found, 666.3 [M+H]⁺. ¹H NMR (400 MHz, Acetonitrile-d₃) δ=8.00 (d, J=1.6 Hz, 1H), 7.66-7.48 (m, 3H), 7.46-7.35 (m, 1H), 7.24 (s, 1H), 6.47 (d, J=2.0 Hz, 1H), 5.84-5.64 (m, 1H), 4.75 (d, J=6.4 Hz, 1H), 4.62-4.52 (m, 1H), 4.46-4.31 (m, 4H), 4.17 (d, J=6.8 Hz, 1H), 3.91-3.86 (m, 3H), 3.77 (d, J=3.2 Hz, 1H), 3.65 (t, J=10.8 Hz, 1H), 3.35-3.24 (m, 1H), 2.91-2.70 (m, 4H), 2.62 (d, J=12.0 Hz, 3H), 2.56-2.49 (m, 3H), 1.45-1.35 (m, 3H).

### Example 48: (2S,4S)-1-(2-Isopropoxybenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

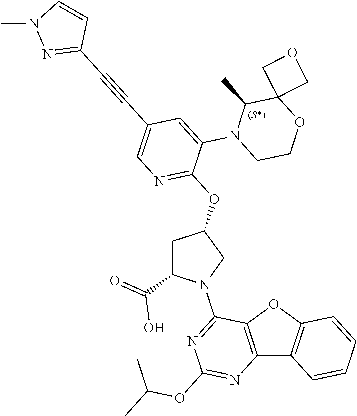

iPrOH (5.0 mL), a stir bar, and NaH (300.0 mg, 7.501 mmol, 60% in mineral oil) were added to a 50 mL round-bottomed flask at 0° C., which was subsequently subjected to three cycles of vacuum and recharging with nitrogen. The resulting yellow heterogeneous mixture was stirred for 30 min and concentrated to dryness in vacuo to give iPrONa as a white solid (620.0 mg). To another 50 mL round-bottomed flask, (2S,4S)-1-(2-chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid (Intermediate 16, 85.0 mg, 0.0981 mmol), iPrOH (10.0 mL), a stir bar and the freshly prepared iPrONa (500.0 mg) were added. The white suspension was stirred at 80° C. overnight to give a yellow heterogeneous mixture, which was cooled to rt, and filtered. The filtrate was subjected to HPLC (Boston Green ODS 5 μm, 150 mm×30 mm, 6 min gradient (50-80% ACN/H₂O (with 0.02% FA)) at 30 mL/min) to afford the title compound, (2S,4S)-1-(2-isopropoxybenzofuro[3,2-d]pyrimidin-4-yl)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid as a white powder (10.5 mg, 15%). LCMS (ESI): mass calcd for C₃₆H₃₇N₇O₇, 679.3; m/z found, 680.3 [M+H]⁺. ¹H NMR (400 MHz, DMSO-d₆) δ 8.02-7.97 (m, 2H), 7.75-7.71 (m, 1H), 7.70-7.61 (m, 2H), 7.48-7.40 (m, 1H), 7.27 (s, 1H), 6.50-6.46 (m, 1H), 5.71-5.63 (m, 1H), 5.30-5.21 (m, 1H), 5.20-4.99 (m, 1H), 4.65 (d, J=6.8 Hz, 1H), 4.60-4.44 (m, 2H), 4.37 (d, J=6.8 Hz, 1H), 4.33-4.26 (m, 1H), 4.26-4.15 (m, 1H), 4.12 (d, J=6.8 Hz, 1H), 3.87 (s, 3H), 3.80-3.74 (m, 1H), 3.70-3.61 (m, 1H), 3.32-3.25 (m, 2H), 2.98-2.94 (m, 1H), 2.73 (d, J=11.6 Hz, 1H), 1.38-1.33 (m, 6H), 0.84 (d, J=6.8 Hz, 3H).

### Example 49: (2S,4S)-1-(2-Cyclopropylbenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

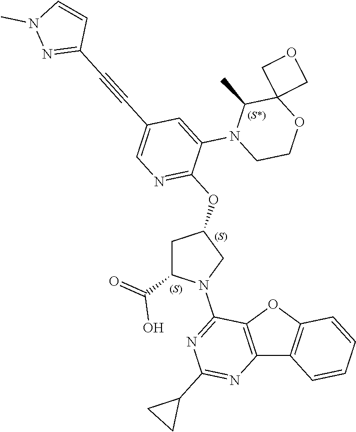

The title compound was prepared in a manner analogous to Example 37, using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-chloro-2-cyclopropylbenzofuro[3,2-d]pyrimidine (Intermediate 6) instead of 4-chloro-2-(difluoromethyl)-7-fluorobenzofuro[3,2-d]pyrimidine (Intermediate 2), and heating to 100° C. for 0.5 h instead of 110° C. for 1 h. LCMS (ESI): mass calcd for C₃₆H₃₅N₇O₆, 661.3; r/z found, 662.3 [M+H]⁺. ¹H NMR (400 MHz, DMSO-d₆) δ 8.07 (d, J=7.6 Hz, 1H), 8.00 (d, J=2.0 Hz, 1H), 7.75-7.69 (m, 2H), 7.68-7.62 (m, 1H), 7.50-7.44 (m, 1H), 7.28 (d, J=2.0 Hz, 1H), 6.48 (d, J=2.0 Hz, 1H), 5.72-5.64 (m, 1H), 5.11-4.94 (m, 1H), 4.68-4.62 (m, 1H), 4.59-4.47 (m, 2H), 4.35 (d, J=6.8 Hz, 1H), 4.32-4.25 (m, 1H), 4.25-4.16 (m, 1H), 4.12 (d, J=6.8 Hz, 1H), 3.87 (s, 3H), 3.81-3.73 (m, 1H), 3.70-3.60 (m, 1H), 3.29 (d, J=3.6 Hz, 1H), 3.03-2.92 (m, 2H), 2.77-2.68 (m, 1H), 2.20-2.12 (m, 1H), 1.10-1.00 (m, 2H), 0.97-0.91 (m, 2H), 0.84 (d, J=6.8 Hz, 3H).

### Example 50: (2S,4S)-1-(2-((2,2-Dimethyl-4-oxo-3,8,11-trioxa-5-azatridecan-13-yl)oxy)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

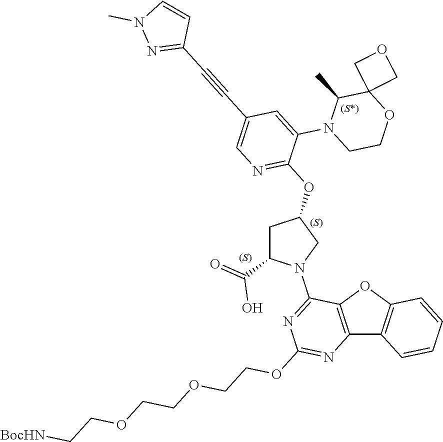

tert-Butyl (2-(2-(2-hydroxyethoxy)ethoxy)ethyl)carbamate (15.0 mg, 0.0602 mmol), a stir bar, DMF (0.4 mL) and NaH (6.0 mg, 0.15 mmol, 60% in mineral oil) were added to a 10 mL round-bottomed flask at 0° C. (ice/water bath), which was subsequently subjected to three cycles of vacuum and recharging with nitrogen, and the resulting yellow heterogeneous mixture was stirred for 1 h. Then (2S,4S)-1-(2-chlorobenzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid (Intermediate 16, 20.0 mg, 0.0298 mmol) was added to the mixture. The yellow suspension was stirred at 0° C. for 2 h, quenched with H₂O (5 mL), and directly concentrated to dryness in vacuo to give a yellow oil, which was subjected to HPLC (Welch Xtimate C18 5 μm, 150 mm×30 mm; 7 min gradient (30-60% ACN/H₂O (with 0.05% aq NH₃+10 mM NH₄HCOs)) at 25 mL/min) to yield (2S,4S)-1-(2-((2,2-dimethyl-4-oxo-3,8,11-trioxa-5-azatridecan-13-yl)oxy)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid as a white powder (4.3 mg, 15%). LCMS (ESI): mass calcd for C₄₄H₅₂N₈O₁₁, 868.4; m/z found, 869.4 [M+H]⁺. ¹H NMR (400 MHz, DMSO-d₆) δ 68.02-7.96 (m, 2H), 7.72 (d, J=2.0 Hz, 1H), 7.70-7.59 (m, 2H), 7.46-7.41 (m, 1H), 7.26 (d, J=2.0 Hz, 1H), 6.51-6.38 (m, 2H), 5.69-5.57 (m, 1H), 5.24-4.88 (m, 1H), 4.67 (d, J=6.8 Hz, 1H), 4.55 (d, J=6.4 Hz, 1H), 4.45 (t, J=4.8 Hz, 2H), 4.41 (d, J=6.8 Hz, 1H). 4.32 (d, J=6.4 Hz, 1H). 4.12 (d, J=6.8 Hz, 1H). 3.89-3.83 (m, 3H), 3.82-3.74 (m, 3H), 3.71-3.65 (m, 1H), 3.64-3.60 (m, 2H), 3.58-3.54 (m, 2H), 3.54-3.46 (m, 1H), 3.44 (t, J=6.4 Hz, 2H), 3.29-3.27 (m, 1H), 2.96-2.91 (m, 2H), 2.74 (d, J=12.0 Hz, 1H), 2.39 (s, 3H), 1.38 (s, 9H), 0.84 (d, J=6.4 Hz, 3H).

### Example 51: (2S,4S)-1-(2-(Difluoromethyl)benzofuro[3,2-d]pyrimidin-4-yl)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid

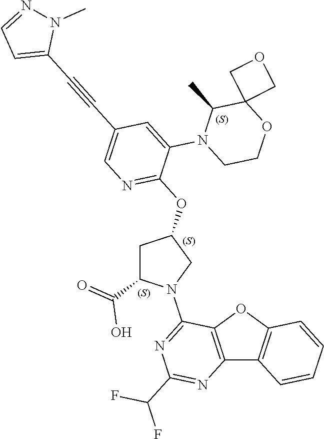

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 5-ethynyl-1-methyl-1H-pyrazole instead of 3-methoxyprop-1-yne, and heating to 90° C. for 2 h instead of 70° C. for 2 h. LCMS (ESI): mass calcd for C₃₄H₃₁F₂N₇O₆, 671.2; m/z found, 672.2 [M+H]⁺. ¹H NMR (400 MHz, CDCl₃) δ 8.23 (d, J=8.0 Hz, 1H), 8.04 (d, J=1.2 Hz, 1H), 7.66-7.57 (m, 2H), 7.52-7.41 (m, 2H), 7.16 (d, J=1.6 Hz, 1H), 6.78-6.48 (m, 2H), 6.37-6.18 (m, 1H), 5.84-5.28 (m, 1H), 5.03-4.90 (m, 1H), 4.85-4.74 (m, 1H), 4.68-4.39 (m, 4H), 4.35-4.10 (m, 1H), 4.01 (s, 3H), 3.92-3.83 (m, 1H), 3.69-3.58 (m, 1H), 3.36-3.24 (m, 1H), 2.98-2.88 (m, 1H), 2.87-2.70 (m, 1H), 2.68-2.60 (m, 1H), 0.93 (d, J=6.8 Hz, 3H).

### Examples 52-69, in Table 10 were prepared in a manner analogous to Example 73, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and the corresponding alkyne

TABLE 10

| Ex # | Structure | Alkyne | MS |
|---|---|---|---|
| 52 | 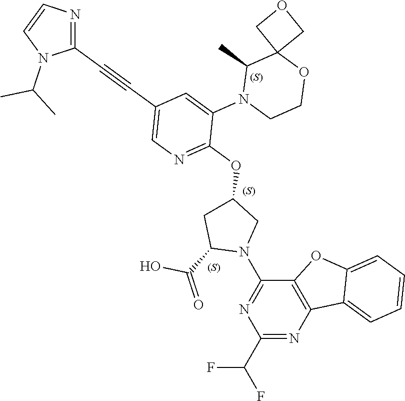 | 2-ethynyl-1-isopropyl-1H- imidazole | mass calcd for C₃₆H₃₅F₂N₇O₆ 699.30 m/z found 700.40 [M + H]⁺ |
| | (2S,4S)-1-(2-(Difluoromethyl)benzofuro[3,2- | | |
| | d]pyrimidin-4-yl)-4-((5-((1-isopropyl-1H-imidazol- | | |
| | 2-yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 53 | 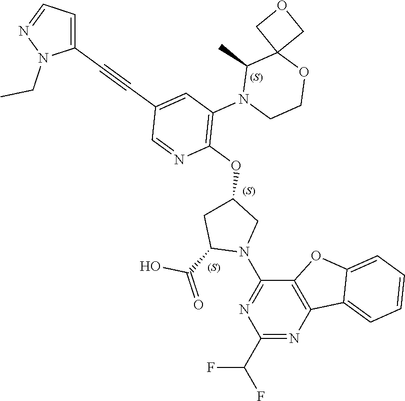 | 1-ethyl-5-ethynyl-1H- pyrazole | mass calcd for C₃₅H₃₃F₂N₇O₆ 685.20 m/z found 686.40 [M + H]⁺ |
| | (2S,4S)-1-(2-(Difluoromethyl)benzofuro[3,2- | | |
| | d]pyrimidin-4-yl)-4-((5-((1-ethyl-1H-pyrazol-5- | | |
| | yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 54 | 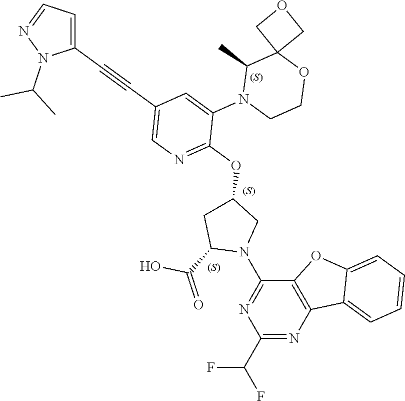 | 5-ethynyl-1-isopropyl-1H- pyrazole | mass calcd for C₃₆H₃₅F₂N₇O₆ 699.30 m/z found 700.50 [M + H]⁺ |
| | (2S,4S)-1-(2-(Difluoromethyl)benzofuro[3,2- | | |
| | d]pyrimidin-4-yl)-4-((5-((1-isopropyl-1H-pyrazol- | | |
| | 5-yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 55 | 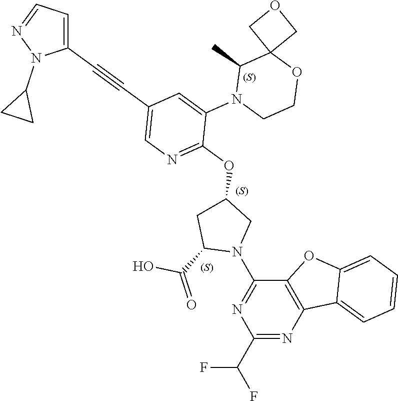 | 1-cyclopropyl-5-ethynyl- 1H-pyrazole | mass calcd for C₃₆H₃₃F₂N₇O₆ 697.20 m/z found 698.40 [M + H]⁺ |
| | (2S,4S)-4-((5-((1-Cyclopropyl-1H-pyrazol-5- | | |
| | yl)ethynyl)-3-((S)-9-methyl-2,5-dioxa-8- | | |
| | azaspiro[3.5]nonan-8-yl)pyridin-2-yl)oxy)-1-(2- | | |
| | (difluoromethyl)benzofuro[3,2-d]pyrimidin-4- | | |
| | yl)pyrrolidine-2-carboxylic acid; | | |
| 56 | 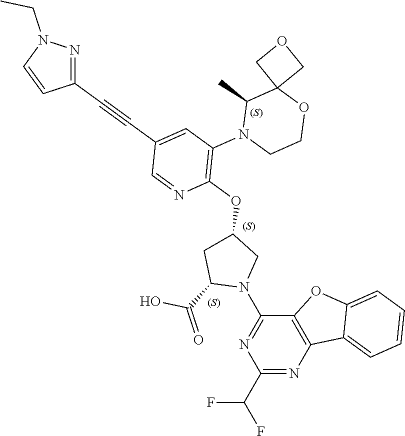 | 1-ethyl-3-ethynyl-1H- pyrazole | mass calcd for C₃₅H₃₃F₂N₇O₆ 685.20 m/z found 686.40 [M + H]⁺ |
| | (2S,4S)-1-(2-(Difluoromethyl)benzofuro[3,2- | | |
| | d]pyrimidin-4-yl)-4-((5-((1-ethyl-1H-pyrazol-3- | | |
| | yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 57 | 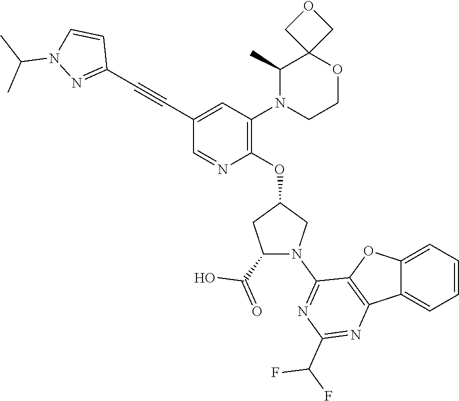 [SMILES: CC(C)n1ccc(C#Cc2cnc(O[C@H]3C[C@@H](C(=O)O)N(c4nc(C(F)F)nc5c4oc4ccccc45)C3)c(N3CCOC4(COC4)[C@@H]3C)c2)n1] | 3-ethynyl-1-isopropyl-1H- pyrazole | mass calcd for C₃₆H₃₅F₂N₇O₆ 699.7 m/z found 700.3 [M + H]⁺ |
| | (2S,4S)-1-(2-(Difluoromethyl)benzofuro[3,2- | | |
| | d]pyrimidin-4-yl)-4-((5-((1-isopropyl-1H-pyrazol- | | |
| | 3-yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 58 | 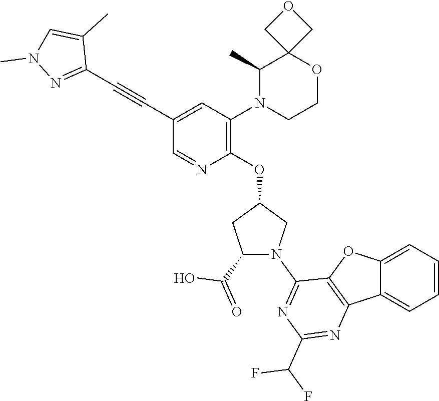 [SMILES: Cc1cn(C)nc1C#Cc1cnc(O[C@H]2C[C@@H](C(=O)O)N(c3nc(C(F)F)nc4c3oc3ccccc34)C2)c(N2CCOC3(COC3)[C@@H]2C)c1] | 3-ethynyl-1,4-dimethyl- 1H-pyrazole | mass calcd for C₃₅H₃₃F₂N₇O₆ 685.20 m/z found 686.30 [M + H]⁺ |
| | (2S,4S)-1-(2-(Difluoromethyl)benzofuro[3,2- | | |
| | d]pyrimidin-4-yl)-4-((5-((1,4-dimethyl-1H- | | |
| | pyrazol-3-yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 59 | 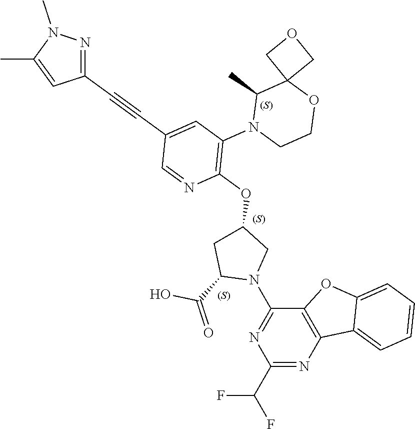 | 3-ethynyl-1,5-dimethyl- 1H-pyrazole | mass calcd for C₃₅H₃₃F₂N₇O₆ 685.20 m/z found 686.30 [M + H]⁺ |
| | (2S,4S)-1-(2-(Difluoromethyl)benzofuro[3,2- | | |
| | d]pyrimidin-4-yl)-4-((5-((1,5-dimethyl-1H- | | |
| | pyrazol-3-yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 60 | 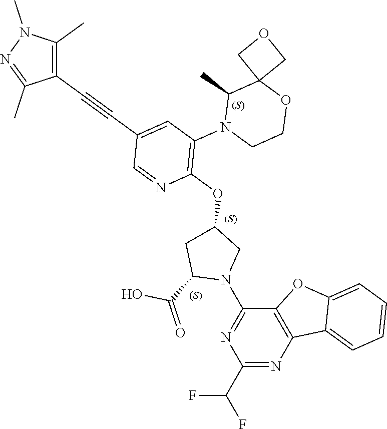 | 4-ethynyl-1,3,5-trimethyl- 1H-pyrazole | mass calcd for C₃₆H₃₅F₂N₇O₆ 699.30 m/z found 700.60 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((1,3,5-trimethyl-1H- | | |
| | pyrazol-4-yl)ethynyl)pyridin-2-yl)oxy)pyrrolidine- | | |
| | 2-carboxylic acid; | | |
| 61 | 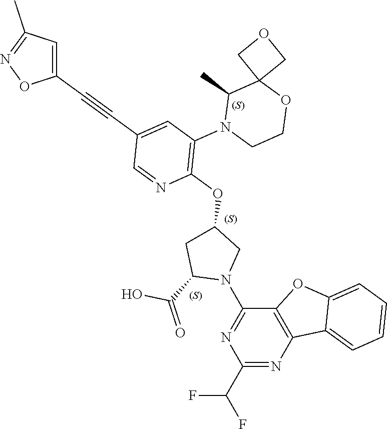 | 5-ethynyl-3- methylisoxazole | mass calcd for C₃₄H₃₀F₂N₆O₇ 672.20 m/z found 673.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((3-methylisoxazol-5- | | |
| | yl)ethynyl)pyridin-2-yl)oxy)pyrrolidine-2- | | |
| | carboxylic acid; | | |
| 62 | 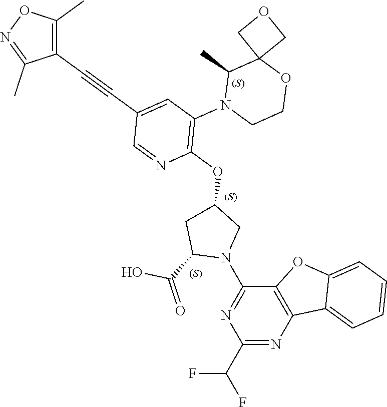 | 4-ethynyl-3,5- dimethylisoxazole | mass calcd for C₃₅H₃₂F₂N₆O₇ 686.20 m/z found 687.40 [M + H]⁺ |
| | (2S,4S)-1-(2-(Difluoromethyl)benzofuro[3,2- | | |
| | d]pyrimidin-4-yl)-4-((5-((3,5-dimethylisoxazol-4- | | |
| | yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 63 | 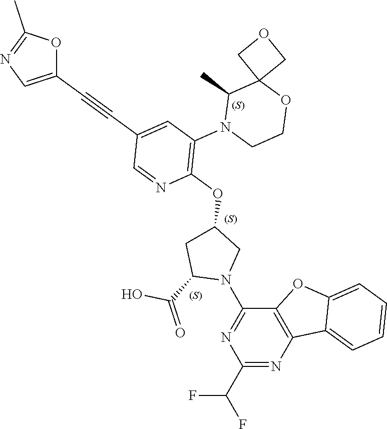 | 5-ethynyl-2-methyloxazole | mass calcd for C₃₄H₃₀F₂N₆O₇ 672.20 m/z found 673.60 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((2-methyloxazol-5- | | |
| | yl)ethynyl)pyridin-2-yl)oxy)pyrrolidine-2- | | |
| | carboxylic acid; | | |
| 64 | 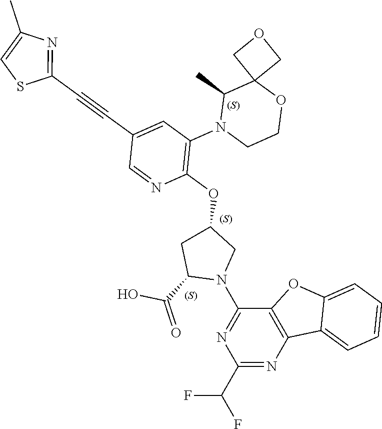 | 2-ethynyl-4- methylthiazole | mass calcd for C₃₄H₃₀F₂N₆O₆S 688.20 m/z found 689.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((4-methylthiazol-2- | | |
| | yl)ethynyl)pyridin-2-yl)oxy)pyrrolidine-2- | | |
| | carboxylic acid; | | |
| 65 | 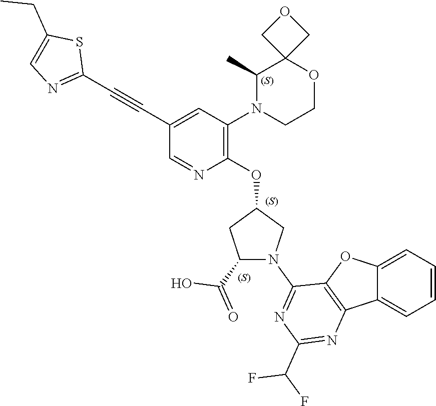 | 5-ethyl-2-ethynylthiazole | mass calcd for C₃₅H₃₂F₂N₆O₆S 702.20 m/z found 703.50 [M + H]⁺ |
| | (2S,4S)-1-(2-(Difluoromethyl)benzofuro[3,2- | | |
| | d]pyrimidin-4-yl)-4-((5-((5-ethylthiazol-2- | | |
| | yl)ethynyl)-3-((S)-9-methyl-2,5-dioxa-8- | | |
| | azaspiro[3.5]nonan-8-yl)pyridin-2- | | |
| | yl)oxy)pyrrolidine-2-carboxylic acid; | | |
| 66 | 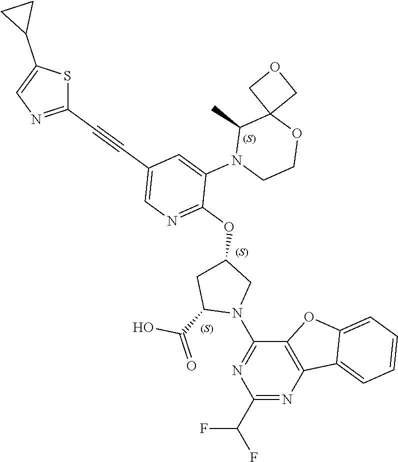 | 2-cyclopropyl-5- ethynylthiazole | mass calcd for C₃₆H₃₂F₂N₆O₆S 714.20 m/z found 715.40 [M + H]⁺ |
| | (2S,4S)-4-((5-((2-Cyclopropylthiazol-5-yl)ethynyl)- | | |
| | 3-((S)-9-methyl-2,5-dioxa-8-azaspiro[3.5]nonan- | | |
| | 8-yl)pyridin-2-yl)oxy)-1-(2- | | |
| | (difluoromethyl)benzofuro[3,2-d]pyrimidin-4- | | |
| | yl)pyrrolidine-2-carboxylic acid; | | |
| 67 | 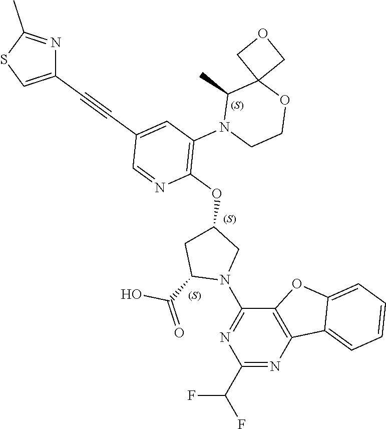 | 4-ethynyl-2- methylthiazole | mass calcd for C₃₄H₃₀F₂N₆O₆S 688.20 m/z found 689.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((2-methylthiazol-4- | | |
| | yl)ethynyl)pyridin-2-yl)oxy)pyrrolidine-2- | | |
| | carboxylic acid; | | |
| 68 | 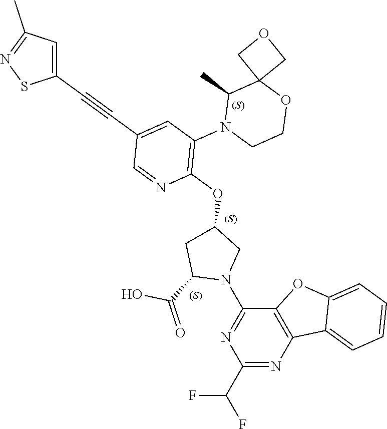 | 5-ethynyl-3- methylisothiazole | mass calcd for C₃₄H₃₀F₂N₆O₆S 688.20 m/z found 689.30 [M + H]⁺ |
| | (2S,4S)-1-(2-(Difluoromethyl)benzofuro[3,2- | | |
| | d]pyrimidin-4-yl)-4-((3-((S)-9-methyl-2,5-dioxa-8- | | |
| | azaspiro[3.5]nonan-8-yl)-5-((3-methylisothiazol- | | |
| | 5-yl)ethynyl)pyridin-2-yl)oxy)pyrrolidine-2- | | |
| | carboxylic acid; | | |
| 69 | 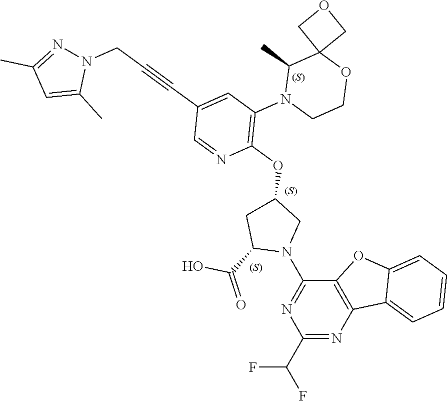 | 3,5-dimethyl-1-(prop-2- yn-1-yl)-1H-pyrazole | mass calcd for C₃₆H₃₅F₂N₇O₆ 699.30 m/z found 700.50 [M + H]⁺ |
| | (2S,4S)-1-(2-(Difluoromethyl)benzofuro[3,2- | | |
| | d]pyrimidin-4-yl)-4-((5-(3-(3,5-dimethyl-1H- | | |
| | pyrazol-1-yl)prop-1-yn-1-yl)-3-((S)-9-methyl-2,5- | | |
| | dioxa-8-azaspiro[3.5]nonan-8-yl)pyridin-2- | | |
| | yl)oxy)pyrrolidine-2-carboxylic acid; | | |

### Example 70: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(pyridin-2-ylethynyl)pyridin-2-yl)oxy)pyrrolidine-2-carboxylic acid

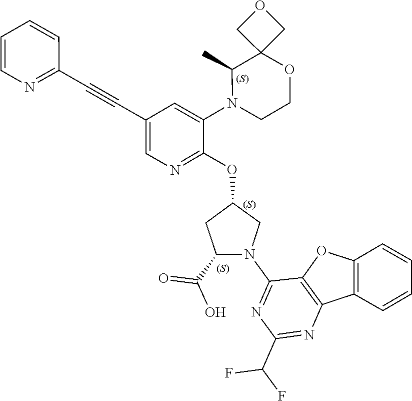

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 2-ethynylpyridine instead of 3-methoxyprop-1-yne, and heating to 90° C. for 2 h instead of 70° C. for 2 h. LCMS (ESI): mass calcd for C₃₅H₃₀F₂N₆O₆, 668.2; r/z found, 669.2 [M+H]⁺. ¹H NMR (400 MHz, CDCl₃) δ 8.64 (d, J=4.8 Hz, 1H), 8.24 (d, J=7.6 Hz, 1H), 8.10 (d, J=2.8 Hz, 1H), 7.75-7.69 (m, 1H), 7.66-7.59 (m, 2H), 7.57-7.53 (m, 1H), 7.50-7.44 (m, 1H), 7.30-7.28 (m, 1H), 6.64 (t, J=54.8 Hz, 1H), 6.30 (s, 1H), 5.76-5.38 (m, 1H), 5.00-4.92 (m, 1H), 4.87-4.56 (m, 3H), 4.56-4.42 (m, 3H), 4.37-4.05 (m, 1H), 3.92-3.84 (m, 1H), 3.67-3.58 (m, 1H), 3.34-3.23 (m, 1H), 2.96-2.69 (m, 2H), 2.67-2.58 (m, 1H), 0.91 (d, J=6.4 Hz, 3H).

### Example 71: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid

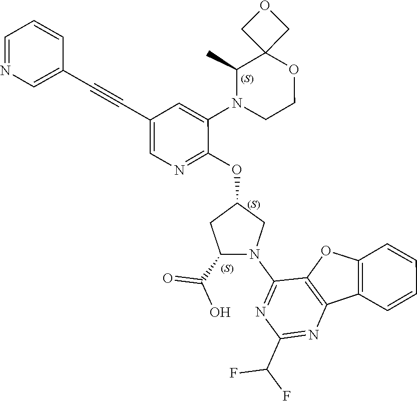

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 3-ethynylpyridine instead of 3-methoxyprop-1-yne. LCMS (ESI): mass calcd for C₃₅H₃₀F₂N₆O₆, 668.2; m/z found, 669.2 [M+H]⁺. ¹H NMR (400 MHz, CDCl₃) δ 8.80 (d, J=1.2 Hz, 1H), 8.59 (dd, J=1.6, 4.8 Hz, 1H), 8.25 (d, J=7.6 Hz, 1H), 8.07 (d, J=1.2 Hz, 1H), 7.87-7.82 (m, 1H), 7.69-7.59 (m, 2H), 7.50-7.44 (m, 1H), 7.36-7.31 (m, 1H), 7.21 (d, J=1.6 Hz, 1H), 6.81-6.49 (m, 1H), 6.31 (s, 1H), 5.80-5.33 (m, 1H), 5.05-4.93 (m, 1H), 4.88-4.75 (m, 1H), 4.75-4.45 (m, 4H), 4.44-4.02 (m, 1H), 3.94-3.87 (m, 1H), 3.72-3.57 (m, 1H), 3.39-3.26 (m, 1H), 3.00-2.90 (m, 1H), 2.90-2.71 (m, 1H), 2.71-2.64 (m, 1H), 0.95 (d, J=6.8 Hz, 3H).

### Example 72: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(pyridin-4-ylethynyl)pyridin-2-yl)oxy)pyrrolidine-2-carboxylic acid

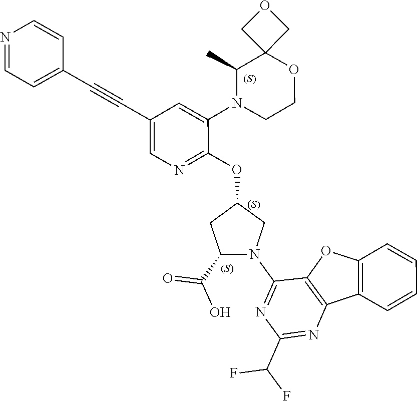

The title compound was prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12) and 4-ethynylpyridine instead of 3-methoxyprop-1-yne, and heating to 90° C. for 2 h instead of 70° C. for 2 h. LCMS (ESI): mass calcd for C₃₅H₃₀F₂N₆O₆, 668.2; r/z found, 669.3 [M+H]⁺. ¹H NMR (400 MHz, CDCl₃) δ 8.65-8.62 (m, 2H), 8.24 (d, J=8.0 Hz, 1H), 8.06 (d, J=1.6 Hz, 1H), 7.67-7.58 (m, 2H), 7.50-7.44 (m, 1H), 7.41-7.38 (m, 2H), 7.19 (d, J=2.0 Hz, 1H), 6.64 (t, J=55.2 Hz, 1H), 6.33-6.26 (m, 1H), 5.77-5.40 (m, 1H), 5.03-4.91 (m, 1H), 4.85-4.72 (m, 1H), 4.70-4.46 (m, 4H), 4.36-3.97 (m, 1H), 3.92-3.86 (m, 1H), 3.68-3.59 (m, 1H), 3.36-3.26 (m, 1H), 2.97-2.90 (m, 1H), 2.88-2.72 (m, 1H), 2.68-2.62 (m, 1H), 0.93 (d, J=6.8 Hz, 3H).

### Example 73: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-((5-methylpyridin-3-yl)ethynyl)pyridin-2-yl)oxy)pyrrolidine-2-carboxylic acid

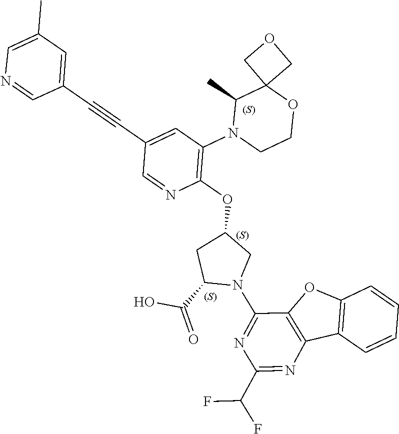

To a 4-mL screw cap vial charged with alkyne, 3-ethynyl-5-methylpyridine (7.2 mg, 62 μmol) and a stir bar was added (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12, 20 mg, 31 μmol) and cesium carbonate (25 mg, 77 μmol). The reaction vial was then taken into a glovebox where dicyclohexyl(2′,6′-diisopropoxy-[1,1′-biphenyl]-2-yl)phosphane (2.9 mg, 6.2 μmol), (2-dicyclohexylphosphino-2′,4′,6′-triisopropyl-1,1′-biphenyl)[2-(2′-amino-1,1′-biphenyl)]palladium(II) methanesulfonate (2.6 mg, 3.1 μmol) and acetonitrile (1.0 mL) were added. The vial was capped, removed from the glovebox, and heated on tumble stirrer at 80° C. for 18 hours. The crude reaction was filtered through an Isolute HM-N, 3-mL cartridge and eluted with acetonitrile (6-mL) followed by dichloromethane (6-mL). The solvent was removed under a stream of nitrogen, crude residues dissolved in a 50/50 mixture of DMSO/methanol and purified by reverse phase HPLC (Waters XSelect CSH C18, 5u, 30×150 mm, formic acid modifier, Gradient 45 to 80% acetonitrile) to afford the title compound (6.97 mg, 32%). LCMS (ESI): mass calcd for C₃₆H₃₂F₂N₆O₆, 682.2; m/z found, 683.2 [M+H]⁺.

Examples 74-88, in Table 11 were prepared in a manner analogous to Example 73, using Intermediate 12 and the corresponding alkyne.

TABLE 11

| Ex # | Structure | Alkyne | MS |
|---|---|---|---|
| 74 | 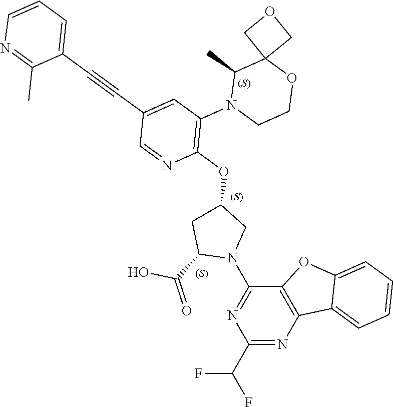 | 3-ethynyl-2- methylpyridine | mass calcd for C₃₆H₃₂F₂N₆O₆ 682.20 m/z found 683.30 [M + H]⁺ |
| | (2S,4S)-1-(2-(Difluoromethyl)benzofuro[3,2-d]pyrimidin-4- | | |
| | yl)-4-((3-((S)-9-methyl-2,5-dioxa-8-azaspiro[3.5]nonan-8-yl)- | | |
| | 5-((2-methylpyridin-3-yl)ethynyl)pyridin-2- | | |
| | yl)oxy)pyrrolidine-2-carboxylic acid; | | |
| 75 | 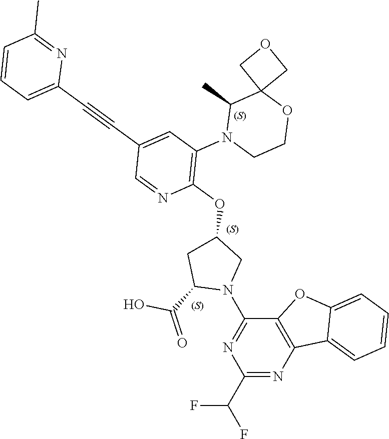 | 2-ethynyl-6- methylpyridine | mass calcd for C₃₆H₃₂F₂N₆O₆ 682.20 m/z found 683.30 [M + H]⁺ |
| | (2S,4S)-1-(2-(Difluoromethyl)benzofuro[3,2-d]pyrimidin-4- | | |
| | yl)-4-((3-((S)-9-methyl-2,5-dioxa-8-azaspiro[3.5]nonan-8-yl)- | | |
| | 5-((6-methylpyridin-2-yl)ethynyl)pyridin-2- | | |
| | yl)oxy)pyrrolidine-2-carboxylic acid; | | |
| 76 | 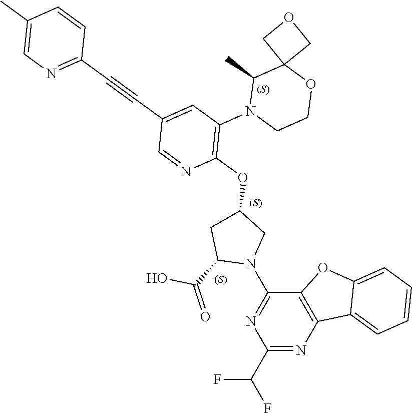 | 2-ethynyl-5- methylpyridine | mass calcd for C₃₆H₃₂F₂N₆O₆ 682.20 m/z found 683.60 [M + H]⁺ |
| | (2S,4S)-1-(2-(Difluoromethyl)benzofuro[3,2-d]pyrimidin-4- | | |
| | yl)-4-((3-((S)-9-methyl-2,5-dioxa-8-azaspiro[3.5]nonan-8-yl)- | | |
| | 5-((5-methylpyridin-2-yl)ethynyl)pyridin-2- | | |
| | yl)oxy)pyrrolidine-2-carboxylic acid; | | |
| 77 | 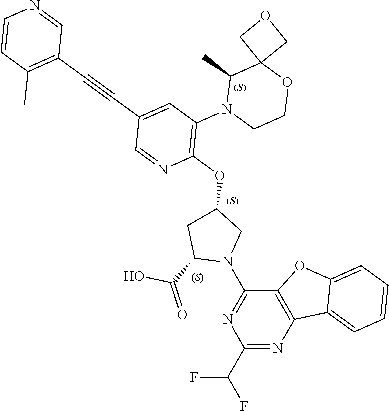 | 3-ethynyl-4- methylpyridine | mass calcd for C₃₆H₃₂F₂N₆O₆ 682.20 m/z found 683.40 [M + H]⁺ |
| | (2S,4S)-1-(2-(Difluoromethyl)benzofuro[3,2-d]pyrimidin-4- | | |
| | yl)-4-((3-((S)-9-methyl-2,5-dioxa-8-azaspiro[3.5]nonan-8-yl)- | | |
| | 5-((4-methylpyridin-3-yl)ethynyl)pyridin-2- | | |
| | yl)oxy)pyrrolidine-2-carboxylic acid; | | |
| 78 | 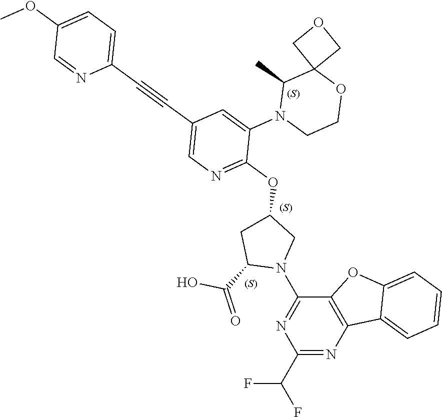 | 2-ethynyl-5 methoxypyridine | mass calcd for C₃₆H₃₂F₂N₆O₇ 698.20 m/z found 699.40 [M + H]⁺ |
| | (2S,4S)-1-(2-(Difluoromethyl)benzofuro[3,2-d]pyrimidin-4- | | |
| | yl)-4-((5-((5-methoxypyridin-2-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 79 | 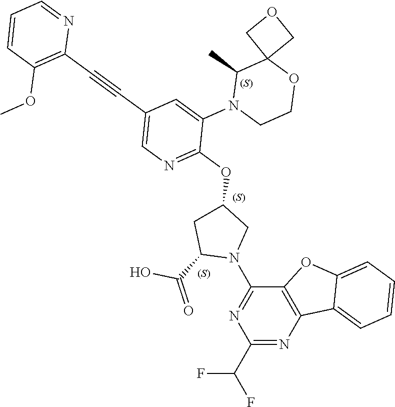 | 2-ethynyl-3- methoxypyridine | mass calcd for C₃₆H₃₂F₂N₆O₇ 698.20 m/z found 699.40 [M + H]⁺ |
| | (2S,4S)-1-(2-(Difluoromethyl)benzofuro[3,2-d]pyrimidin-4- | | |
| | yl)-4-((5-((3-methoxypyridin-2-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 80 | 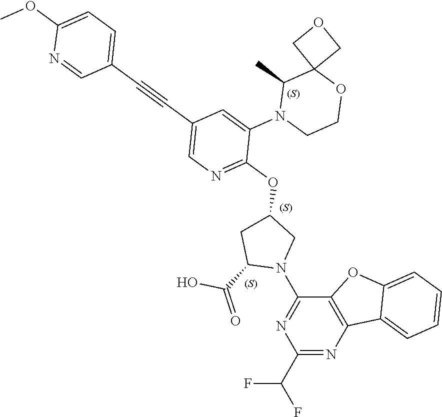 | 5-ethynyl-2- methoxypyridine | mass calcd for C₃₆H₃₂F₂N₆O₇ 698.20 m/z found 699.40 [M + H]⁺ |
| | (2S,4S)-1-(2-(Difluoromethyl)benzofuro[3,2-d]pyrimidin-4- | | |
| | yl)-4-((5-((6-methoxypyridin-3-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 81 | 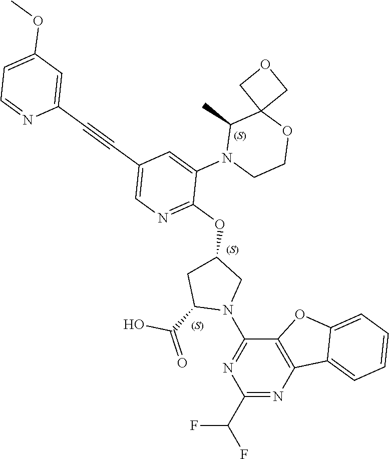 | 2-ethynyl-4- methoxypyridine | mass calcd for C₃₆H₃₂F₂N₆O₇ 698.20 m/z found 699.40 [M + H]⁺ |
| | (2S,4S)-1-(2-(Difluoromethyl)benzofuro[3,2-d]pyrimidin-4- | | |
| | yl)-4-((5-((4-methoxypyridin-2-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 82 | 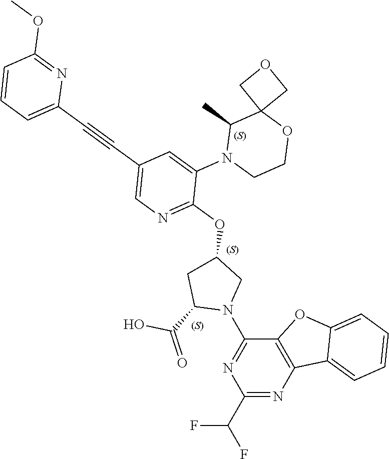 | 2-ethynyl-6- methoxypyridine | mass calcd for C₃₆H₃₂F₂N₆O₇ 698.20 m/z found 699.60 [M + H]⁺ |
| | (2S,4S)-1-(2-(Difluoromethyl)benzofuro[3,2-d]pyrimidin-4- | | |
| | yl)-4-((5-((6-methoxypyridin-2-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 83 | 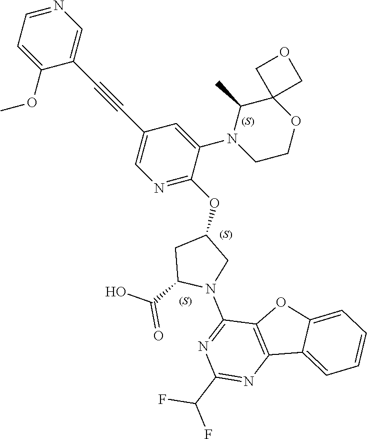 | 3-ethynyl-4- methoxypyridine | mass calcd for C₃₆H₃₂F₂N₆O₇ 698.20 m/z found 699.50 [M + H]⁺ |
| | (2S,4S)-1-(2-(Difluoromethyl)benzofuro[3,2-d]pyrimidin-4- | | |
| | yl)-4-((5-((4-methoxypyridin-3-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 84 | 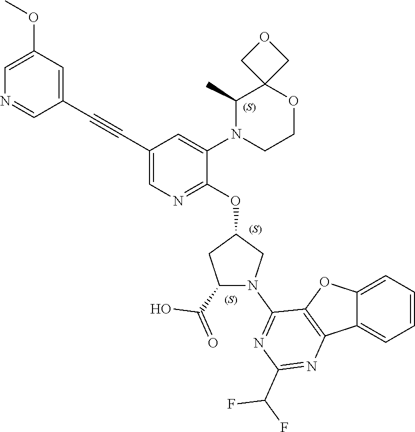 | 3-ethynyl-5- methoxypyridine | mass calcd for C₃₆H₃₂F₂N₆O₇ 698.20 m/z found 699.60 [M + H]⁺ |
| | (2S,4S)-1-(2-(Difluoromethyl)benzofuro[3,2-d]pyrimidin-4- | | |
| | yl)-4-((5-((5-methoxypyridin-3-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 85 | 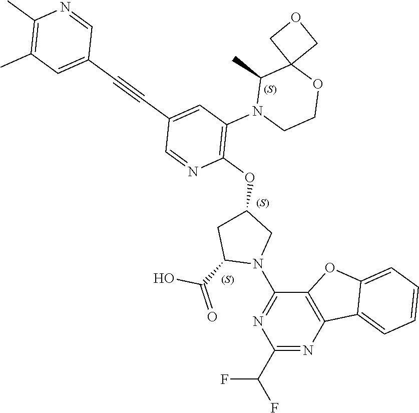 | 5-ethynyl-2,3- dimethylpyridine | mass calcd for C₃₇H₃₄F₂N₆O₆ 696.30 m/z found 697.30 [M + H]⁺ |
| | (2S,4S)-1-(2-(Difluoromethyl)benzofuro[3,2-d]pyrimidin-4- | | |
| | yl)-4-((5-((5,6-dimethylpyridin-3-yl)ethynyl)-3-((S)-9-methyl- | | |
| | 2,5-dioxa-8-azaspiro[3.5]nonan-8-yl)pyridin-2- | | |
| | yl)oxy)pyrrolidine-2-carboxylic acid; | | |
| 86 | 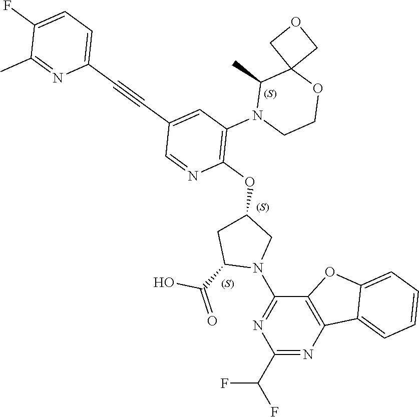 | 6-ethynyl-3- fluoro-2- methylpyridine | mass calcd for C₃₆H₃₁F₃N₆O₆ 700.20 m/z found 701.30 [M + H]⁺ |
| | (2S,4S)-1-(2-(Difluoromethyl)benzofuro[3,2-d]pyrimidin-4- | | |
| | yl)-4-((5-((5-fluoro-6-methylpyridin-2-yl)ethynyl)-3-((S)-9- | | |
| | methyl-2,5-dioxa-8-azaspiro[3.5]nonan-8-yl)pyridin-2- | | |
| | yl)oxy)pyrrolidine-2-carboxylic acid; | | |
| 87 | 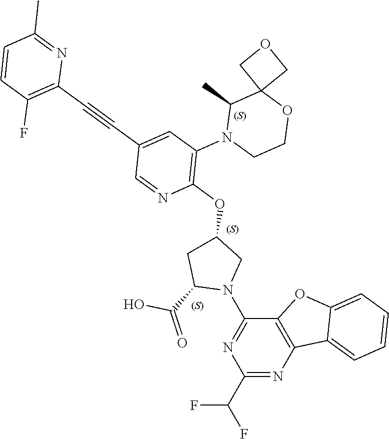 | 2-ethynyl-3- fluoro-6- methylpyridine | mass calcd for C₃₆H₃₁F₃N₆O₆ 688.20 m/z found 689.30 [M + H]⁺ |
| | (2S,4S)-1-(2-(Difluoromethyl)benzofuro[3,2-d]pyrimidin-4- | | |
| | yl)-4-((5-((3-fluoro-6-methylpyridin-2-yl)ethynyl)-3-((S)-9- | | |
| | methyl-2,5-dioxa-8-azaspiro[3.5]nonan-8-yl)pyridin-2- | | |
| | yl)oxy)pyrrolidine-2-carboxylic acid; | | |
| 88 | 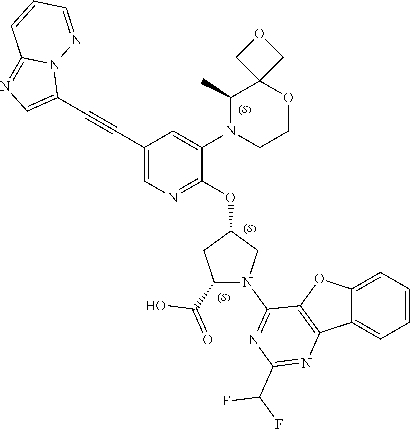 | 3- ethynylimidazo[1, 2-b]pyridazine | mass calcd for C₃₆H₃₀F₂N₈O₆ 700.20 m/z found 701.40 [M + H]⁺ |
| | (2S,4S)-1-(2-(Difluoromethyl)benzofuro[3,2-d]pyrimidin-4- | | |
| | yl)-4-((5-(imidazo[1,2-b]pyridazin-3-ylethynyl)-3-((S)-9- | | |
| | methyl-2,5-dioxa-8-azaspiro[3.5]nonan-8-yl)pyridin-2- | | |
| | yl)oxy)pyrrolidine-2-carboxylic acid; | | |

### Example 89: (2S,4S)-4-((5-((1-Methyl-1H-pyrazol-5-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-pyrrolidine-2-carboxylic acid

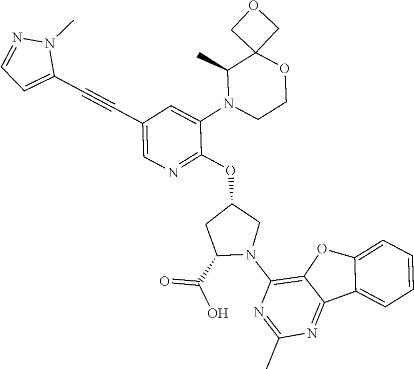 [SMILES: Cc1nc(N2C[C@@H](Oc3ncc(C#Cc4ccnn4C)cc3N3CCOC4(COC4)[C@@H]3C)C[C@H]2C(=O)O)c2oc3ccccc3c2n1]

A mixture of (2S,4S)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate (Intermediate 25, 260.0 mg, 0.4581 mmol), DMSO (3.0 mL), 4-chloro-2-methylbenzofuro[3,2-d]pyrimidine (50.0 mg, 0.229 mmol), and DIEA (1.20 mL, 7.06 mmol) was heated at 110° C. for 1 h. The reaction mixture was cooled to rt, passed through a syringe filter (0.22 μm nylon membrane), then subjected to HPLC (Phenomenex Gemini NX C18, 3 μm, 150 mm×30 mm; 7 min gradient (20-50% ACN/H₂O (with 0.05% of 25% aq NH₃+10 mM NH₄HCO₃)) at 25 mL/min) to afford the title compound, (2S,4S)-4-((5-((1-methyl-1H-pyrazol-5-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-yl)pyrrolidine-2-carboxylic acid as a white powder (55.4 mg, 37%). LCMS (ESI): mass calcd for C₃₄H₃₃N₇O₆, 635.2; m/z found, 636.3 [M+H]⁺. ¹H NMR (400 MHz, DMSO-d₆) δ 8.09-8.05 (m, 2H), 7.72-7.63 (m, 2H), 7.50-7.49 (m, 1H), 7.48-7.43 (m, 1H), 7.35 (d, J=2.0 Hz, 1H), 6.58 (d, J=2.0 Hz, 1H), 5.71-5.65 (m, 1H), 5.22-5.09 (m, 1H), 4.68-4.64 (m, 1H), 4.57-4.51 (m, 2H), 4.39-4.35 (m, 1H), 4.32-4.26 (m, 1H), 4.22-4.11 (m, 2H), 3.95 (s, 3H), 3.80-3.75 (m, 1H), 3.70-3.62 (m, 1H), 3.33-3.24 (m, 1H), 3.01-2.96 (m, 1H), 2.79-2.72 (m, 1H), 2.62-2.57 (m, 1H), 2.55 (s, 3H), 0.86 (d, J=6.8 Hz, 3H).

### Example 91: (2S,4S)-4-((5-((1,3-Dimethyl-1H-pyrazol-5-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-yl)pyrrolidine-2-carboxylic acid

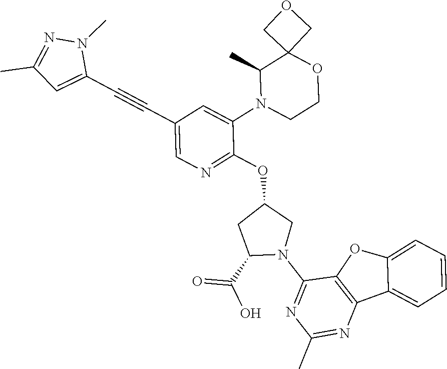 [SMILES: Cc1cc(C#Cc2cnc(O[C@H]3C[C@@H](C(=O)O)N(c4nc(C)nc5c4oc4ccccc45)C3)c(N3CCOC4(COC4)[C@@H]3C)c2)n(C)n1]

A mixture of 4-chloro-2-methylbenzofuro[3,2-d]pyrimidine (60.0 mg, 0.274 mmol), DMSO (2.00 mL), (2S,4S)-4-((5-((1,3-dimethyl-1H-pyrazol-5-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate (Intermediate 20, 165.5 mg, 0.2533 mmol), and DIEA (0.19 mL, 1.1 mmol) was stirred at 110° C. for 1 h. The reaction mixture was cooled to rt, and subjected to HPLC (Phenomenex Gemini NX C18, 5 μm, 150 mm×30 mm; 7 min gradient (30-60% ACN/H₂O (with 0.2% FA)) at 25 mL/min) to give the title compound, (2S,4S)-4-((5-((1,3-Dimethyl-1H-pyrazol-5-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-yl)pyrrolidine-2-carboxylic acid (52.0 mg, 24%). LCMS (ESI): mass calcd for C₃₅H₃₅N₇O₆, 649.3; r/z found, 650.3 [M+H]⁺. ¹H NMR (400 MHz, DMSO-d₆) δ 8.09-8.04 (m, 2H), 7.72-7.63 (m, 2H), 7.50-7.43 (m, 1H), 7.33 (d, J=2.0 Hz, 1H), 6.34 (s, 1H), 5.71-5.63 (m, 1H), 5.21-5.09 (m, 1H), 4.69-4.62 (m, 1H), 4.58-4.49 (m, 2H), 4.40-4.34 (m, 1H), 4.32-4.24 (m, 1H), 4.23-4.15 (m, 1H), 4.15-4.10 (m, 1H), 3.86 (s, 3H), 3.80-3.74 (m, 1H), 3.69-3.62 (m, 1H), 3.29-3.27 (m, 1H), 2.98-2.96 (m, 1H), 2.76-2.73 (m, 1H), 2.61-2.57 (m, 1H), 2.55 (s, 3H), 2.18 (s, 3H), 0.85 (d, J=6.4 Hz, 3H).

Examples 90, 92-95, and 97 in Table 12 were prepared in a manner analogous to Example 37, using 4-chloro-2-methylbenzofuro[3,2-d]pyrimidine and the corresponding acid.

TABLE 12

| Ex # | Structure | Acid | MS and ¹H NMR |
|---|---|---|---|
| 90 | 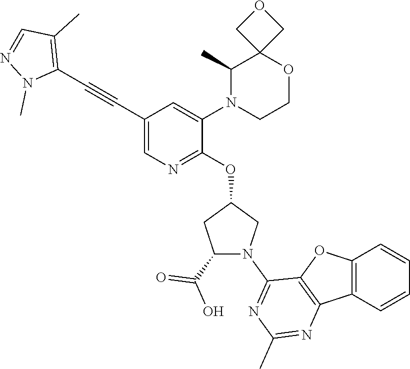 [SMILES: Cc1nc(N2C[C@@H](Oc3ncc(C#Cc4c(C)cnn4C)cc3N3CCOC4(COC4)[C@@H]3C)C[C@H]2C(=O)O)c2oc3ccccc3c2n1] | (2S,4S)-4-((5-((1,4- dimethyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (Intermediate 22) | LCMS (ESI): mass calcd for C₃₅H₃₅N₇O₆, 649.3; m/z found, 650.3 [M + H]⁺. ¹H NMR (400 MHz, DMSO-d₆) δ 8.10 − 8.05 (m, 2H), 7.73 − 7.63 (m, 2H), 7.50 − 7.44 (m, 1H), 7.36 − 7.33 (m, 2H), 5.71 − 5.64 (m, 1H), 5.20 − 5.10 (m, 1H), 4.71 − 4.64 (m, 1H), 4.60 − 4.50 (m, 2H), 4.42 − 4.35 (m, 1H), 4.34 − 4.27 (m, 1H), 4.23 − 4.10 (m, 2H), 3.90 (s, 3H), 3.81 − 3.75 (m, 1H), 3.72 − 3.61 (m, 1H), 3.35 − 3.29 (m, 1H), 2.98 − 2.94 (m, 1H), 2.80 − 2.74 (m, 1H), 2.62 − 2.58 (m, 1H), 2.56 (s, 3H), 2.13 (s, 3H), 0.87 (d, J = 6.4 Hz, 3H). |
| | (2S,4S)-4-((5-((1,4-Dimethyl-1H-pyrazol- | | |
| | 5-yl)ethynyl)-3-((S)-9-methyl-2,5-dioxa- | | |
| | 8-azaspiro[3.5]nonan-8-yl)pyridin-2- | | |
| | yl)oxy)-1-(2-methylbenzofuro[3,2- | | |
| | d]pyrimidin-4-yl)pyrrolidine-2-carboxylic | | |
| | acid; | | |
| 92 | 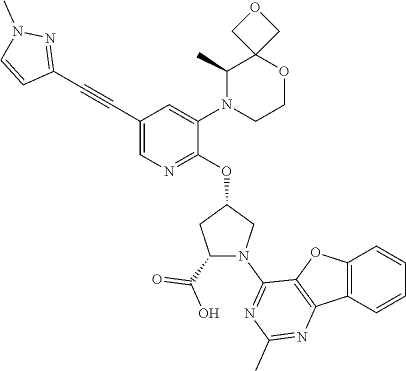 [SMILES: Cc1nc(N2C[C@@H](Oc3ncc(C#Cc4ccn(C)n4)cc3N3CCOC4(COC4)[C@@H]3C)C[C@H]2C(=O)O)c2oc3ccccc3c2n1] | (2S,4S)-4-((5-((1- methyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 23) | LCMS (ESI): mass calcd for C₃₄H₃₃N₇O₆, 635.2; m/z found, 636.3 [M + H]⁺. ¹H NMR (400 MHz, DMSO-d₆) δ 8.08 (d, J = 8.0 Hz, 1H), 8.01 (d, J = 1.6 Hz, 1H), 7.74 − 7.69 (m, 2H), 7.69 − 7.64 (m, 1H), 7.50 − 7.45 (m, 1H), 7.28 (d, J = 1.6 Hz, 1H), 6.49 (d, J = 2.4 Hz, 1H), 5.70 − 5.64 (m, 1H), 5.20 − 5.11 (m, 1H), 4.68 − 4.64 (m, 1H), 4.59 − 4.50 (m, 2H), 4.40 − 4.35 (m, 1H), 4.33 − 4.25 (m, 1H), 4.22 − 4.12 (m, 2H), 3.88 (s, 3H), 3.82 − 3.74 (m, 1H), 3.71 − 3.62 (m, 1H), 3.33 − 3.28 (m, 1H), 2.99 − 2.97 (m, 1H), 2.79 − 2.72 (m, 1H), 2.64 − 2.58 (m, 1H), 2.56 (s, 3H), 0.86 (d, J = 6.8 Hz, 3H). |
| | (2S,4S)-4-((5-((1-Methyl-1H-pyrazol-3- | | |
| | yl)ethynyl)-3-((S)-9-methyl-2,5-dioxa-8- | | |
| | azaspiro[3.5]nonan-8-yl)pyridin-2- | | |
| | yl)oxy)-1-(2-methylbenzofuro[3,2- | | |
| | d]pyrimidin-4-yl)pyrrolidine-2-carboxylic | | |
| | acid | | |
| 93 | 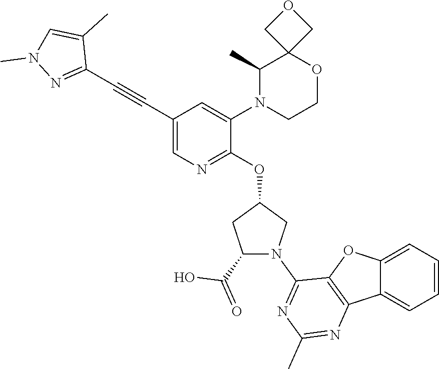 [SMILES: Cc1nc(N2C[C@@H](Oc3ncc(C#Cc4nn(C)cc4C)cc3N3CCOC4(COC4)[C@@H]3C)C[C@H]2C(=O)O)c2oc3ccccc3c2n1] | (2S,4S)-4-((5-((1,4- dimethyl-1H-pyrazol-3- yl)ethynyl)-3-((S)-9- methyl-2,5-dioxa-8- azaspiro [3.5]nonan-8- yl)pyridin-2- yl)oxy)pyrrolidine-2- carboxylic acid trifluoroacetate (Intermediate 19) | LCMS (ESI): mass calcd for C₃₅H₃₅N₇O₆, 649.3; m/z found, 650.3 [M + H]⁺. ¹H NMR (400 MHz, DMSO-d₆) δ 8.07 (d, J = 7.6 Hz, 1H), 8.02 − 7.99 (m, 1H), 7.73 − 7.61 (m, 2H), 7.52 (s, 1H), 7.49 − 7.43 (m, 1H), 7.28 − 7.25 (m, 1H), 5.75 − 5.55 (m, 1H), 5.21 − 5.08 (m, 1H), 4.69 − 4.62 (m, 1H), 4.58 − 4.48 (m, 2H), 4.41 − 4.34 (m, 1H), 4.33 − 4.24 (m, 1H), 4.21 − 4.09 (m, 2H), 3.81 (s, 3H), 3.79 − 3.73 (m, 1H), 3.70 − 3.61 (m, 1H), 3.31 − 3.27 (m, 1H), 2.97 − 2.93 (m, 1H), 2.78 − 2.72 (m, 1H), 2.62 − 2.56 (m, 1H), 2.55 (s, 3H), 2.10 (s, 3H), 0.85 (d, J = 6.4 Hz, 3H). |
| | (2S,4S)-4-((5-((1,4-Dimethyl-1H-pyrazol- | | |
| | 3-yl)ethynyl)-3-((S)-9-methyl-2,5-dioxa- | | |
| | 8-azaspiro[3.5]nonan-8-yl)pyridin-2- | | |
| | yl)oxy)-1-(2-methylbenzofuro[3,2- | | |
| | d]pyrimidin-4-yl)pyrrolidine-2-carboxylic | | |
| | acid; | | |
| 94 | 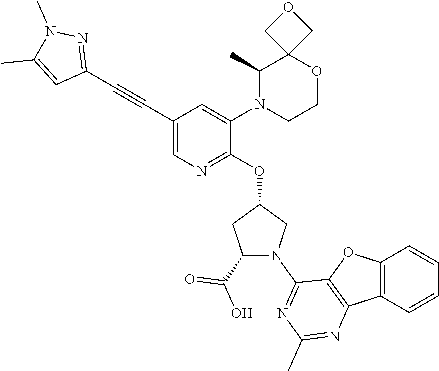 [SMILES: Cc1nc(N2C[C@@H](Oc3ncc(C#Cc4cc(C)n(C)n4)cc3N3CCOC4(COC4)[C@@H]3C)C[C@H]2C(=O)O)c2oc3ccccc3c2n1] | (2S,4S)-4-((5-((1,5- dimethyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 24) | LCMS (ESI): mass calcd for C₃₅H₃₅N₇O₆, 649.3; m/z found, 650.3 [M + H]⁺. ¹H NMR (400 MHz, DMSO-d₆) δ 8.07 (d, J = 7.6 Hz, 1H), 8.01 − 7.96 (m, 1H), 7.73 − 7.63 (m, 2H), 7.50 − 7.44 (m, 1H), 7.28 − 7.24 (m, 1H), 6.26 (s, 1H), 5.69 − 5.63 (m, 1H), 5.23 − 5.08 (m, 1H), 4.68 − 4.62 (m, 1H), 4.58 − 4.47 (m, 2H), 4.40 − 4.33 (m, 1H), 4.32 − 4.23 (m, 1H), 4.22 − 4.15 (m, 1H), 4.15 − 4.10 (m, 1H), 3.80 − 3.76 (m, 1H), 3.75 (s, 3H), 3.70 − 3.61 (m, 1H), 3.31 − 3.25 (m, 1H), 2.98 − 2.94 (m, 1H), 2.76 − 2.71 (m, 1H), 2.61 − 2.57 (m, 1H), 2.55 (s, 3H), 2.27 (s, 3H), 0.85 (d, J = 6.8 Hz, 3H). |
| | (2S,4S)-4-((5-((1,5-Dimethyl-1H-pyrazol- | | |
| | 3-yl)ethynyl)-3-((S)-9-methyl-2,5-dioxa- | | |
| | 8-azaspiro[3.5]nonan-8-yl)pyridin-2- | | |
| | yl)oxy)-1-(2-methylbenzofuro[3,2- | | |
| | d]pyrimidin-4-yl)pyrrolidine-2-carboxylic | | |
| | acid | | |
| 95 | 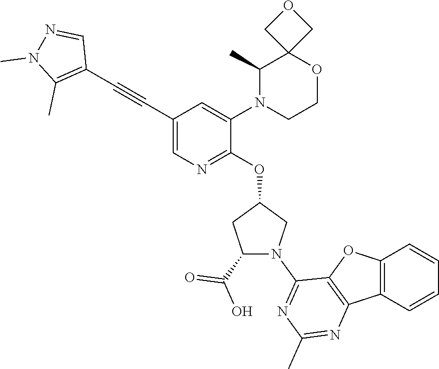 [SMILES: Cc1nc(N2C[C@@H](Oc3ncc(C#Cc4cnn(C)c4C)cc3N3CCOC4(COC4)[C@@H]3C)C[C@H]2C(=O)O)c2oc3ccccc3c2n1] | (2S,4S)-4-((5-((1,5- dimethyl-1H-pyrazol-4- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (Intermediate 27) | LCMS (ESI): mass calcd for C₃₅H₃₅N₇O₆, 649.3; m/z found, 650.3 [M + H]⁺. ¹H NMR (400 MHz, DMSO-d₆) δ 8.06 (d, J = 7.6 Hz, 1H), 7.95 (d, J = 1.6 Hz, 1H), 7.74 − 7.68 (m, 1H), 7.68 − 7.62 (m, 1H), 7.53 (s, 1H), 7.49 − 7.41 (m, 1H), 7.23 (d, J = 1.6 Hz, 1H), 5.71 − 5.57 (m, 1H), 5.21 − 5.02 (m, 1H), 4.70 − 4.63 (m, 1H), 4.60 − 4.47 (m, 2H), 4.41 − 4.33 (m, 1H), 4.31 − 4.22 (m, 1H), 4.21 − 4.08 (m, 2H), 3.80 − 3.73 (m, 4H), 3.70 − 3.61 (m, 1H), 3.30 − 3.24 (m, 1H), 2.98 − 2.94 (m, 1H), 2.77 − 2.71 (m, 1H), 2.61 − 2.57 (m, 1H), 2.55 (s, 3H), 2.36 (s, 3H), 0.84 (d, J = 6.8 Hz, 3H). |
| | (2S,4S)-4-((5-((1,5-Dimethyl-1H-pyrazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)-1-(2-methylbenzofuro[3,2- | | |
| | d]pyrimidin-4-yl)pyrrolidine-2-carboxylic | | |
| 97 | 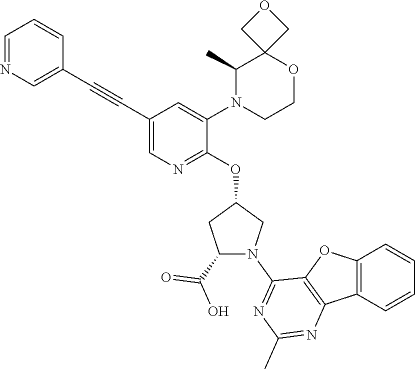 [SMILES: Cc1nc(N2C[C@@H](Oc3ncc(C#Cc4cccnc4)cc3N3CCOC4(COC4)[C@@H]3C)C[C@H]2C(=O)O)c2oc3ccccc3c2n1] | (2S,4S)-4-((3-((S)-9- methyl-2,5-dioxa-8- azaspiro[3.5]nonan-8- yl)-5-(pyridin-3- ylethynyl)pyridin-2- yl)oxy)pyrrolidine-2- carboxylic acid trifluoroacetate (Intermediate 39) | LCMS (ESI): mass calcd for C₃₅H32N606, 632.2; m/z found, 633.3 [M + H]⁺. ¹H NMR (400 MHz, DMSO-d6) δ 8.77 − 8.73 (m, 1H), 8.58 (d, J = 4.8 Hz, 1H), 8.09 − 8.04 (m, 2H), 7.98 − 7.92 (m, 1H), 7.72 − 7.62 (m, 2H), 7.49 − 7.43 (m, 2H), 7.36 − 7.33 (m, 1H), 5.69 − 5.63 (m, 1H), 5.20 − 5.07 (m, 1H), 4.70 − 4.62 (m, 1H), 4.61 − 4.51 (m, 2H), 4.41 − 4.35 (m, 1H), 4.34 − 4.25 (m, 1H), 4.23 − 4.03 (m, 2H), 3.82 − 3.73 (m, 1H), 3.73 − 3.60 (m, 1H), 3.32 − 3.25 (m, 1H), 3.00 − 2.96 (m, 1H), 2.78 − 2.72 (m, 1H), 2.62 − 2.56 (m, 1H), 2.55 (s, 3H), 0.85 (d, J = 6.8 Hz, 3H). |
| | (2S,4S)-4-((3-((S)-9-Methyl-2,5-dioxa-8- | | |
| | azaspiro[3.5]nonan-8-yl)-5-(pyridin-3- | | |
| | ylethynyl)pyridin-2-yl)oxy)-1-(2- | | |
| | methylbenzofuro[3,2-d]pyrimidin-4- | | |
| | yl)pyrrolidine-2-carboxylic acid | | |

### Example 96: (2S,4S)-4-((5-((1,3-Dimethyl-1H-pyrazol-4-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-yl)pyrrolidine-2-carboxylic acid

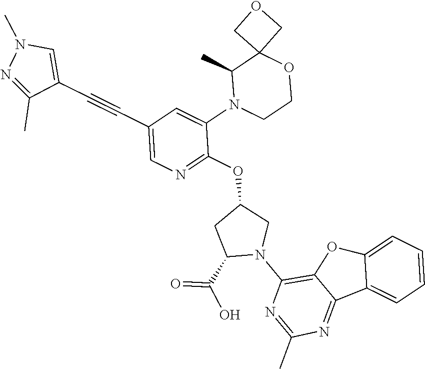 [SMILES: Cc1nc(N2C[C@@H](Oc3ncc(C#Cc4cn(C)nc4C)cc3N3CCOC4(COC4)[C@@H]3C)C[C@H]2C(=O)O)c2oc3ccccc3c2n1]

A mixture of 4-chloro-2-methylbenzofuro[3,2-d]pyrimidine (50.0 mg, 0.229 mmol), DMSO (0.50 mL), (2S,4S)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)pyrrolidine-2-carboxylic acid trifluoroacetate (Intermediate 26, 125.0 mg, 0.2149 mmol), and DIEA (0.12 mL, 0.71 mmol) was stirred at 100° C. for 1 h. The reaction mixture was passed through a syringe filter (0.22 μm nylon membrane) and subjected to HPLC (Phenomenex Gemini NX C18, 3 μm, 150 mm×30 mm; 7 min gradient (20-50% ACN/H₂O (with 0.05% of 25% aq NH₃+10 mM NH₄HCO₃)) at 25 mL/min) to afford the title compound, (2S,4S)-4-((5-((1,3-dimethyl-1H-pyrazol-4-yl)ethynyl)-3-((S)-9-methyl-2,5-dioxa-8-azaspiro[3.5]nonan-8-yl)pyridin-2-yl)oxy)-1-(2-methylbenzofuro[3,2-d]pyrimidin-4-yl)pyrrolidine-2-carboxylic acid as an off-white powder (40.0 mg, 27%). LCMS (ESI): mass calcd for C₃₅H₃₅N₇O₆, 649.3; m/z found, 650.3 [M+H]⁺. ¹H NMR (400 MHz, DMSO-d₆) δ 8.07 (d, J=8.0 Hz, 1H), 7.95 (d, J=1.6 Hz, 1H), 7.85 (s, 1H), 7.73-7.68 (m, 1H), 7.68-7.62 (m, 1H), 7.50-7.43 (m, 1H), 7.22 (d, J=2.0 Hz, 1H), 5.69-5.59 (m, 1H), 5.20-5.08 (m, 1H), 4.68-4.62 (m, 1H), 4.57-4.54 (m, 1H), 4.54-4.46 (m, 1H), 4.40-4.33 (m, 1H), 4.31-4.23 (m, 1H), 4.21-4.14 (m, 1H), 4.14-4.10 (m, 1H), 3.77 (s, 3H), 3.77-3.73 (m, 1H), 3.69-3.59 (m, 1H), 3.30-3.23 (m, 1H), 3.01-2.95 (m, 1H), 2.75-2.70 (m, 1H), 2.60-2.56 (m, 1H), 2.55 (s, 3H), 2.24 (s, 3H), 0.84 (d, J=6.8 Hz, 3H).

### Example 98: (2S,4S)-4-((3-((S)-9-Methyl-2,5-dioxa-8-azaspiro[3.5]nonan-8-yl)-5-((2-methylpyridin-3-yl)ethynyl)pyridin-2-yl)oxy)-1-(2-methylbenzofuro[3,2-d]pyrimidin-4-yl)pyrrolidine-2-carboxylic acid

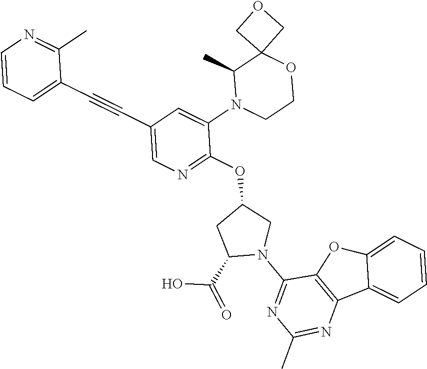 [SMILES: Cc1nc(N2C[C@@H](Oc3ncc(C#Cc4cccnc4C)cc3N3CCOC4(COC4)[C@@H]3C)C[C@H]2C(=O)O)c2oc3ccccc3c2n1]

A mixture of 4-chloro-2-methylbenzofuro[3,2-d]pyrimidine (47.5 mg, 0.217 mmol), DMSO (2.00 mL), (2S,4S)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid trifluoroacetate (Intermediate 21, 116.3 mg, 0.2010 mmol), and DIEA (0.15 mL, 0.89 mmol) was stirred at 110° C. for 1 h. The resulting brown solution was cooled to rt, and subjected to HPLC (Phenomenex Gemini NX C18, 5 μm, 150 mm×30 mm; 7 min gradient (17-47% ACN/H₂O (with 0.2% FA)) at 25 mL/min) followed by SFC (DAICEL CHIRALPAK AD, 10 μm, 250 mm×30 mm; 4 min isocratic (50% EtOH (with 0.1% of 25% aq NH₃)/CO₂) at 150 mL/min (100 bar); column temp 35° C.) to afford the title compound (2S,4S)-4-((3-((S)-9-methyl-2,5-dioxa-8-azaspiro[3.5]nonan-8-yl)-5-((2-methylpyridin-3-yl)ethynyl)pyridin-2-yl)oxy)-1-(2-methylbenzofuro[3,2-d]pyrimidin-4-yl)pyrrolidine-2-carboxylic acid as a white powder (36.0 mg, 22⁰/). LCMS (ESI): mass calcd for C₃₆H₃₄N₆O₆, 646.3; r/z found, 647.3 [M+H]⁺. ¹H NMR (400 MHz, DMSO-d₆) δ 8.47-8.42 (m, 1H), 8.10-8.05 (m, 2H), 7.89-7.84 (m, 1H), 7.73-7.63 (m, 2H), 7.50-7.43 (m, 1H), 7.33 (d, J=1.6 Hz, 1H), 7.29-7.23 (m, 1H), 5.70-5.64 (m, 1H), 5.23-5.07 (m, 1H), 4.68-4.63 (m, 1H), 4.58-4.55 (m, 1H), 4.54-4.46 (m, 1H), 4.39-4.35 (m, 1H), 4.33-4.26 (m, 1H), 4.24-4.15 (m, 1H), 4.15-4.11 (m, 1H), 3.81-3.74 (m, 1H), 3.70-3.62 (m, 1H), 3.30-3.27 (m, 1H), 2.98-2.95 (m, 1H), 2.78-2.73 (m, 1H), 2.68 (s, 3H), 2.62-2.57 (m, 1H), 2.55 (s, 31H), 0.86 (d, J=6.4 Hz, 31H).

### Examples 99-106 in Table 13: were prepared in a manner analogous to Example 37, using 4-chloro-2-ethylbenzofuro[3,2-d]pyrimidine (intermediate 18) and the corresponding acid

TABLE 13

| Ex # | Structure | Acid | MS and ¹H-NMR |
|---|---|---|---|
| 99 | 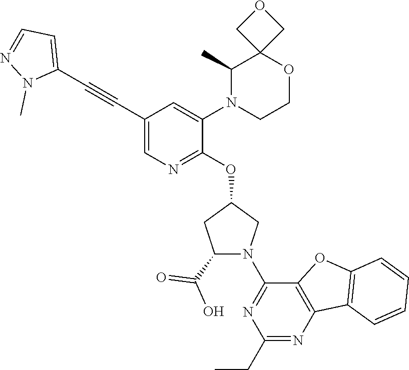 [SMILES: CCc1nc(N2C[C@@H](Oc3ncc(C#Cc4ccnn4C)cc3N3CCOC4(COC4)[C@@H]3C)C[C@H]2C(=O)O)c2oc3ccccc3c2n1] | (2S,4S)-4-((5-((1- methyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (Intermediate 25) | LCMS (ESI): mass calcd for C₃₅H₃₅N₇O₆, 649.3; m/z found, 650.3 [M + H]⁺. ¹H NMR (400 MHz, DMSO-d₆) δ 8.13 − 8.04 (m, 2H), 7.72 − 7.61 (m, 2H), 7.50 − 7.48 (m, 1H), 7.47 − 7.42 (m, 1H), 7.33 (d, J = 1.2 Hz, 1H), 6.57 (d, J = 2.0 Hz, 1H), 5.72 − 5.61 (m, 1H), 5.19 − 5.04 (m, 1H), 4.69 − 4.63 (m, 1H), 4.58 − 4.48 (m, 2H), 4.38 − 4.34 (m, 1H), 4.32 − 4.27 (m, 1H), 4.25 − 4.14 (m, 1H), 4.13 − 4.10 (m, 1H), 3.94 (s, 3H), 3.79 − 3.75 (m, 1H), 3.70 − 3.60 (m, 2H), 3.00 − 2.97 (m, 1H), 2.85 − 2.80 (m, 2H), 2.75 − 2.70 (m, 1H), 2.59 − 2.53 (m, 1H), 1.31 (t, J = 7.6 Hz, 3H), 0.84 (d, J = 6.8 Hz, 3H). |
| | (2S,4S)-1-(2-Ethylbenzofuro[3,2- | | |
| | d]pyrimidin-4-yl)-4-((5-((1-methyl-1H- | | |
| | pyrazol-5-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 100 | 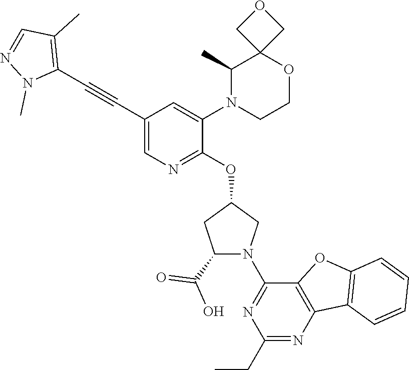 [SMILES: CCc1nc(N2C[C@@H](Oc3ncc(C#Cc4c(C)cnn4C)cc3N3CCOC4(COC4)[C@@H]3C)C[C@H]2C(=O)O)c2oc3ccccc3c2n1] | (2S,4S)-4-((5-((1,4- dimethyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (Intermediate 22) | LCMS (ESI): mass calcd for C₃₆H₃₇N₇O₆, 663.3; m/z found, 664.3 [M + H]⁺. ¹H NMR (400 MHz, DMSO-d₆) δ 8.11 − 8.07 (m, 2H), 7.74 − 7.62 (m, 2H), 7.51 − 7.44 (m, 1H), 7.37 − 7.32 (m, 2H), 5.73 − 5.66 (m, 1H), 5.24 − 5.04 (m, 1H), 4.70 − 4.65 (m, 1H), 4.61 − 4.49 (m, 2H), 4.39 − 4.36 (m, 1H), 4.35 − 4.28 (m, 1H), 4.27 − 4.17 (m, 1H), 4.17 − 4.10 (m, 1H), 3.90 − (s, 3H), 3.82 − 3.74 (m, 1H), 3.73 3.59 (m, 1H), 3.35 − 3.26 (m, 1H), 3.03 − 2.95 (m, 1H), 2.89 − 2.79 (m, 2H), 2.79 − 2.73 (m, 1H), 2.61 − 2.55 (m, 1H), 2.13 (s, 3H), 1.33 (t, J = 7.6 Hz, 3H), 0.86 (d, J = 6.8 Hz, 3H). |
| | (2S,4S)-4-((5-((1,4-Dimethyl-1H-pyrazol- | | |
| | 5-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |
| 101 | 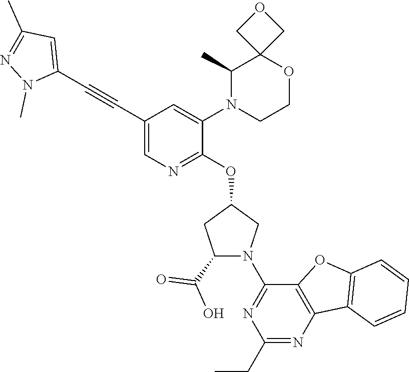 [SMILES: CCc1nc(N2C[C@@H](Oc3ncc(C#Cc4cc(C)nn4C)cc3N3CCOC4(COC4)[C@@H]3C)C[C@H]2C(=O)O)c2oc3ccccc3c2n1] | (2S,4S)-4-((5-((1,3- dimethyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 20) | LCMS (ESI): mass calcd for C₃₆H₃₇N₇O₆, 663.3; m/z found, 664.4 [M + H]⁺. ¹H NMR (400 MHz, DMSO-d₆) δ 8.10 − 8.05 (m, 2H), 7.72 − 7.62 (m, 2H), 7.49 − 7.44 (m, 1H), 7.33 (d, J = 2.0 Hz, 1H), 6.34 (s, 1H), 5.70 − 5.64 (m, 1H), 5.17 − 5.08 (m, 1H), 4.70 − 4.63 (m, 1H), 4.60 − 4.51 (m, 2H), 4.39 − 4.33 (m, 1H), 4.33 − 4.27 (m, 1H), 4.25 − 4.16 (m, 1H), 4.15 − 4.08 (m, 1H), 3.86 (s, 3H), 3.81 − 3.73 (m, 1H), 3.70 − 3.61 (m, 1H), 3.32 − 3.30 (m, 1H), 2.97 − 2.94 (m, 1H), 2.86 − 2.80 (m, 2H), 2.77 − 2.70 (m, 1H), 2.62 − 2.56 (m, 1H), 2.18 (s, 3H), 1.32 (t, J = 7.6 Hz, 3H), 0.85 (d, J = 6.4 Hz, 3H). |
| | (2S,4S)-4-((5-((1,3-Dimethyl-1H-pyrazol- | | |
| | 5-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |
| 102 | 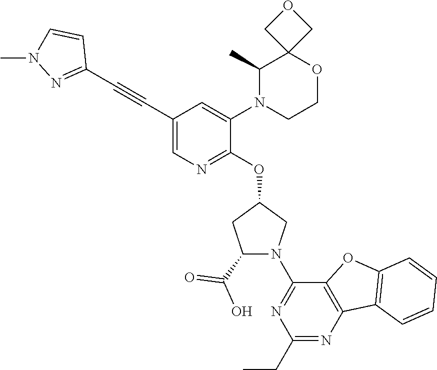 [SMILES: CCc1nc(N2C[C@@H](Oc3ncc(C#Cc4ccn(C)n4)cc3N3CCOC4(COC4)[C@@H]3C)C[C@H]2C(=O)O)c2oc3ccccc3c2n1] | (2S,4S)-4-((5-((1- methyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 23) | LCMS (ESI): mass calcd for C₃₅H₃₅N₇O₆, 649.3; m/z found, 650.3 [M + H]⁺. ¹H NMR (400 MHz, DMSO-d₆) δ 8.08 (d, J = 8.0 Hz, 1H), 8.00 (d, J = 1.6 Hz, 1H), 7.75 − 7.68 (m, 2H), 7.68 − 7.62 (m, 1H), 7.50 − 7.43 (m, 1H), 7.27 (d, J = 1.2 Hz, 1H), 6.48 (d, J = 2.0 Hz, 1H), 5.72 − 5.61 (m, 1H), 5.22 − 5.04 (m, 1H), 4.69 − 4.62 (m, 1H), 4.62 − 4.49 (m, 2H), 4.40 − 4.34 (m, 1H), 4.33 − 4.25 (m, 1H), 4.24 − 4.16 (m, 1H), 4.14 − 4.09 (m, 1H), 3.87 (s, 3H), 3.81 − 3.73 (m, 1H), 3.71 − 3.61 (m, 1H), 3.32 − 3.28 (m, 1H), 2.97 − 2.94 (m, 1H), 2.86 − 2.80 (m, 2H), 2.76 − 2.71 (m, 1H), 2.61 − 2.53 (m, 1H), 1.32 (t, J = 8.0 Hz, 3H), 0.84 (d, J = 6.4 Hz, 3H). |
| | (2S,4S)-1-(2-Ethylbenzofuro[3,2- | | |
| | d]pyrimidin-4-yl)-4-((5-((1-methyl-1H- | | |
| | pyrazol-3-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 103 | 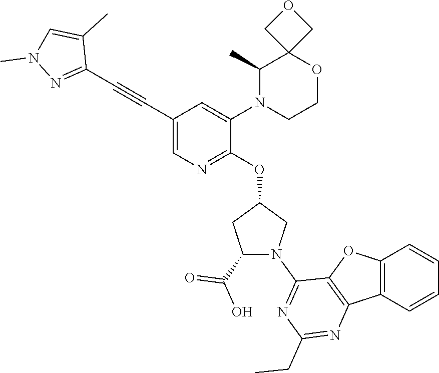 [SMILES: CCc1nc(N2C[C@@H](Oc3ncc(C#Cc4nn(C)cc4C)cc3N3CCOC4(COC4)[C@@H]3C)C[C@H]2C(=O)O)c2oc3ccccc3c2n1] | (2S,4S)-4-((5-((1,4- dimethyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 28) | LCMS (ESI): mass calcd for C₃₆H₃₇N₇O₆, 663.3; m/z found, 664.3 [M + H]⁺. ¹H NMR (400 MHz, DMSO-d₆) δ 12.39 (br s, 1H), 8.08 (d, J = 7.6 Hz, 1H), 8.01 (d, J = 1.2 Hz, 1H), 7.74 − 7.69 (m, 1H), 7.68 − 7.62 (m, 1H), 7.52 (s, 1H), 7.49 − 7.44 (m, 1H), 7.27 (d, J = 1.2 Hz, 1H), 5.73 − 5.63 (m, 1H), 5.23 − 5.05 (m, 1H), 4.70 − 4.63 (m, 1H), 4.60 − 4.50 (m, 2H), 4.38 − 4.34 (m, 1H), 4.33 − 4.26 (m, 1H), 4.25 − 4.18 (m, 1H), 4.14 − 4.09 (m, 1H), 3.81 (s, 3H), 3.79 − 3.74 (m, 1H), 3.69 − 3.62 (m, 1H), 3.33 − 3.25 (m, 1H), 3.00 − 2.95 (m, 1H), 2.87 − 2.80 (m, 2H), 2.77 − 2.72 (m, 1H), 2.60 − 2.53 (m, 1H), 2.10 (s, 3H), 1.32 (t, J = 7.6 Hz, 3H), 0.85 (d, J = 6.4 Hz, 3H). |
| | (2S,4S)-4-((5-((1,4-Dimethyl-1H-pyrazol- | | |
| | 3-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |
| 104 | 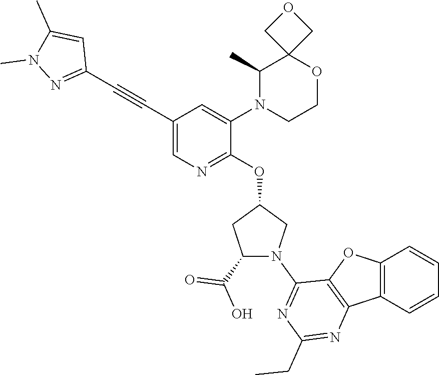 [SMILES: CCc1nc(N2C[C@@H](Oc3ncc(C#Cc4cc(C)n(C)n4)cc3N3CCOC4(COC4)[C@@H]3C)C[C@H]2C(=O)O)c2oc3ccccc3c2n1] | (2S,4S)-4-((5-((1,5- dimethyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 24) | LCMS (ESI): mass calcd for C₃₆H₃₇N₇O₆, 663.3; m/z found, 664.3 [M + H]⁺. ¹H NMR (400 MHz, DMSO-d₆) δ 8.08 (d, J = 7.6 Hz, 1H), 7.98 (d, J = 1.2 Hz, 1H), 7.74 − 7.69 (m, 1H), 7.68 − 7.63 (m, 1H), 7.51 − 7.43 (m, 1H), 7.26 (d, J = 1.2 Hz, 1H), 6.27 (s, 1H), 5.71 − 5.63 (m, 1H), 5.23 − 5.04 (m, 1H), 4.69 − 4.63 (m, 1H), 4.60 − 4.50 (m, 2H), 4.38 − 4.34 (m, 1H), 4.33 − 4.25 (m, 1H), 4.24 − 4.15 (m, 1H), 4.14 − 4.10 (m, 1H), 3.80 − 3.75 (m, 1H), 3.75 (s, 3H), 3.70 − 3.61 (m, 1H), 3.33 − 3.24 (m, 1H), 3.02 − 2.96 (m, 1H), 2.88 − 2.79 (m, 2H), 2.77 − 2.70 (m, 1H), 2.60 − 2.54 (m, 1H), 2.27 (s, 3H), 1.32 (t, J = 7.6 Hz, 3H), 0.84 (d, J = 6.4 Hz, 3H). |
| | (2S,4S)-4-((5-((1,5-Dimethyl-1H-pyrazol- | | |
| | 3-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |
| 105 | 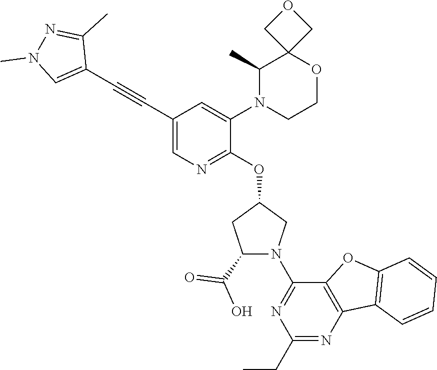 [SMILES: CCc1nc(N2C[C@@H](Oc3ncc(C#Cc4cn(C)nc4C)cc3N3CCOC4(COC4)[C@@H]3C)C[C@H]2C(=O)O)c2oc3ccccc3c2n1] | (2S,4S)-4-((5-((1,3- dimethyl-1H-pyrazol-4- yl)ethynyl)-3-((S)-9- methyl-2,5-dioxa-8- azaspiro [3.5]nonan-8- yl)pyridin-2- yl)oxy)pyrrolidine-2- carboxylic acid trifluoroacetate (intermediate 26) | LCMS (ESI): mass calcd for C₃₆H₃₇N₇O₆, 663.3; m/z found, 664.3 [M + H]⁺. ¹H NMR (400 MHz, DMSO-d₆) δ 8.08 (d, J = 7.6 Hz, 1H), 7.95 (d, J = 1.6 Hz, 1H), 7.85 (s, 1H), 7.74 − 7.62 (m, 2H), 7.50 − 7.43 (m, 1H), 7.22 (d, J = 1.6 Hz, 1H), 5.71 − 5.61 (m, 1H), 5.20 − 5.04 (m, 1H), 4.69 − 4.63 (m, 1H), 4.61 − 4.47 (m, 2H), 4.39 − 4.34 (m, 1H), 4.32 − 4.24 (m, 1H), 4.24 − 4.16 (m, 1H), 4.15 − 4.10 (m, 1H), 3.78 (s, 3H), 3.76 − 3.74 (m, 1H), 3.71 − 3.59 (m, 1H), 3.33 − 3.23 (m, 1H), 3.02 − 2.96 (m, 1H), 2.87 − 2.79 (m, 2H), 2.77 − 2.69 (m, 1H), 2.59 − 2.52 (m, 1H), 2.25 (s, 3H), 1.33 (t, J = 7.6 Hz, 3H), 0.84 (d, J = 6.4 Hz, 3H). |
| | (2S,4S)-4-((5-((1,3-Dimethyl-1H-pyrazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |
| 106 | 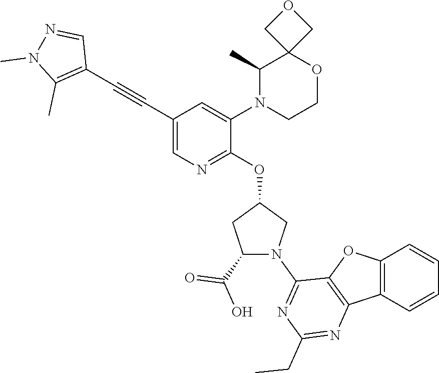 [SMILES: CCc1nc(N2C[C@@H](Oc3ncc(C#Cc4cnn(C)c4C)cc3N3CCOC4(COC4)[C@@H]3C)C[C@H]2C(=O)O)c2oc3ccccc3c2n1] | (2S,4S)-4-((5-((1,5- dimethyl-1H-pyrazol-4- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 27) | LCMS (ESI): mass calcd for C₃₆H₃₇N₇O₆, 663.3; m/z found, 664.3 [M + H]⁺. ¹H NMR (400 MHz, DMSO-d₆) δ 8.08 (d, J = 7.6 Hz, 1H), 7.96 (d, J = 2.0 Hz, 1H), 7.73 − 7.69 (m, 1H), 7.68 − 7.62 (m, 1H), 7.53 (s, 1H), 7.49 − 7.43 (m, 1H), 7.23 (d, J = 2.0 Hz, 1H), 5.68 − 5.59 (m, 1H), 5.18 − 5.04 (m, 1H), 4.69 − 4.63 (m, 1H), 4.61 − 4.48 (m, 2H), 4.40 − 4.33 (m, 1H), 4.32 − 4.24 (m, 1H), 4.23 − 4.15 (m, 1H), 4.14 − 4.10 (m, 1H), 3.80 − 3.73 (m, 4H), 3.71 − 3.61 (m, 1H), 3.31 − 3.26 (m, 1H), 2.99 − 2.95 (m, 1H), 2.86 − 2.80 (m, 2H), 2.76 − 2.70 (m, 1H), 2.60 − 2.55 (m, 1H), 2.37 (s, 3H), 1.32 (t, J = 7.6 Hz, 3H), 0.84 (d, J = 6.4 Hz, 3H). |
| | (2S,4S)-4-((5-((1,5-Dimethyl-1H-pyrazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid | | |

### Example 107: (2S,4S)-1-(2-Ethylbenzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid

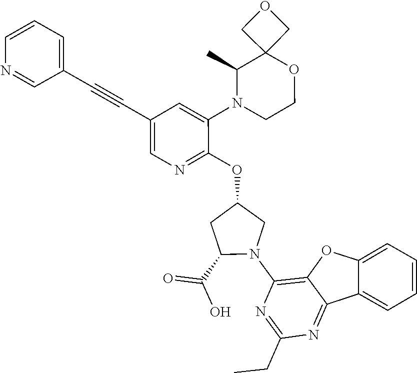 [SMILES: CCc1nc(N2C[C@@H](Oc3ncc(C#Cc4cccnc4)cc3N3CCOC4(COC4)[C@@H]3C)C[C@H]2C(=O)O)c2oc3ccccc3c2n1]

A mixture of 4-chloro-2-ethylbenzofuro[3,2-d]pyrimidine (Intermediate 18, 30.0 mg, 0.124 mmol), (2S,4S)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid trifluoroacetate (Intermediate 39, 100.0 mg, 0.1523 mmol), DIEA (63 μL, 0.37 mmol), and DMSO (3.0 mL) was stirred at 110° C. for 2 h. The reaction mixture was then cooled to rt, passed through a syringe filter (0.45 μm nylon membrane), and the filtrate subjected to HPLC (Phenomenex Gemini NX C18, 3 μm, 150 mm×30 mm, 7 min gradient (23-53% ACN/H₂O (with 0.05% of 25% aq NH₃+10 mM NH₄HCOs)) at 25 mL/min) followed by SFC (DAICEL CHIRALCEL OJ, 10 μm, 250 mm×30 mm, 7.5 min isocratic (40% EtOH (with 0.1% of 25% aq NH₃)/CO₂) at 80 mL/min (100 bar); column temp 35° C.) to afford the title compound (2S,4S)-1-(2-ethylbenzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid as a white powder (26.6 mg, 31%). LCMS (ESI): mass calcd for C₃₆H₃₄N₆O₆, 646.3; m/z found, 647.3 [M+H]⁺. ¹H NMR (400 MHz, DMSO-d₆) δ 8.76-8.74 (m, 1H), 8.61-8.56 (m, 1H), 8.10-8.06 (m, 2H), 7.97-7.93 (m, 1H), 7.74-7.69 (m, 1H), 7.68-7.63 (m, 1H), 7.49-7.46 (m, 1H), 7.46-7.43 (m, 1H), 7.35 (d, J=1.6 Hz, 1H), 5.73-5.65 (m, 1H), 5.19-5.08 (m, 1H), 4.69-4.64 (m, 1H), 4.60-4.51 (m, 2H), 4.39-4.34 (m, 1H), 4.33-4.27 (m, 1H), 4.26-4.18 (m, 1H), 4.15-4.10 (m, 1H), 3.82-3.75 (m, 1H), 3.71-3.62 (m, 1H), 3.34-3.25 (m, 1H), 3.03-2.96 (m, 1H), 2.87-2.80 (m, 2H), 2.78-2.72 (m, 1H), 2.61-2.53 (m, 1H), 1.33 (t, J=7.6 Hz, 3H), 0.85 (d, J=6.8 Hz, 3H).

### Example 108: (2S,4S)-1-(2-Ethylbenzofuro[3,2-d]pyrimidin-4-yl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid

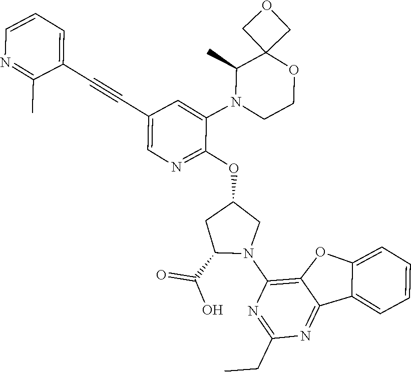 [SMILES: CCc1nc(N2C[C@@H](Oc3ncc(C#Cc4cccnc4C)cc3N3CCOC4(COC4)[C@@H]3C)C[C@H]2C(=O)O)c2oc3ccccc3c2n1]

A mixture of 4-chloro-2-ethylbenzofuro[3,2-d]pyrimidine (Intermediate 18, 90.0 mg, 0.380 mmol), DMSO (2.00 mL), (2S,4S)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid trifluoroacetate (Intermediate 21, 201.3 mg, 0.3479 mmol), and DIEA (0.30 mL, 1.8 mmol) was stirred at 110° C. for 1 h. The resulting brown solution was cooled to rt, and subjected to HPLC (Phenomenex Gemini NX C18, 5 μm, 150 mm×40 mm; 8 min gradient (20-50% ACN/H₂O (with 0.2% FA)) at 55 mL/min) to afford the title compound (2S,4S)-1-(2-ethylbenzofuro[3,2-d]pyrimidin-4-yl)-4-((3-((S)-9-methyl-2,5-dioxa-8-azaspiro[3.5]nonan-8-yl)-5-((2-methylpyridin-3-yl)ethynyl)pyridin-2-yl)oxy)pyrrolidine-2-carboxylic acid as an off-white powder (66.3 mg, 22%). LCMS (ESI): mass calcd for C₃₇H₃₆N₆O₆, 660.3; n/z found, 661.3 [M+H]⁺. ¹H NMR (400 MHz, DMSO-d₆) δ 8.47-8.43 (m, 1H), 8.10-8.06 (m, 2H), 7.89-7.84 (m, 1H), 7.74-7.68 (m, 1H), 7.68-7.63 (m, 1H), 7.50-7.44 (m, 1H), 7.32 (d, J=1.6 Hz, 1H), 7.29-7.24 (m, 1H), 5.72-5.64 (m, 1H), 5.25-5.02 (m, 1H), 4.68-4.64 (m, 1H), 4.61-4.51 (m, 2H), 4.39-4.35 (m, 1H), 4.34-4.27 (m, 1H), 4.26-4.16 (m, 1H), 4.15-4.09 (m, 1H), 3.81-3.74 (m, 1H), 3.70-3.61 (m, 1H), 3.30-3.28 (m, 1H), 3.00-2.98 (m, 1H), 2.86-2.81 (m, 2H), 2.78-2.72 (m, 1H), 2.68 (s, 3H), 2.62-2.55 (m, 1H), 1.32 (t, J=7.6 Hz, 3H), 0.85 (d, J=6.8 Hz, 3H).

### Example 110: (2S,4S)-4-((5-((1-Ethyl-1H-pyrazol-5-yl)ethynyl)-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid

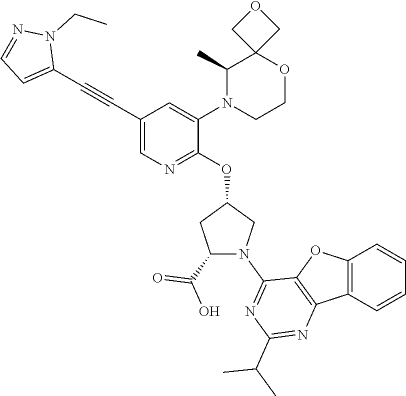 [SMILES: CCn1nccc1C#Cc1cnc(O[C@H]2C[C@@H](C(=O)O)N(c3nc(C(C)C)nc4c3oc3ccccc34)C2)c(N2CCOC3(COC3)[C@@H]2C)c1]

Under nitrogen, a mixture of (2S,4S)-4-((5-ethynyl-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid (Intermediate 34, 100.0 mg, 0.1630 mmol), ACN (4.0 mL), 5-bromo-1-ethyl-1H-pyrazole (85.0 mg, 0.486 mmol), XPhos Pd G3 (14.0 mg, 0.0165 mmol), Xphos (14.0 mg, 0.0294 mmol), and Cs₂CO₃ (133.0 mg, 0.4082 mmol) was stirred at 70° C. for 2 h. The reaction mixture was cooled to rt, passed through a syringe filter (0.45 μm nylon membrane), and subjected to HPLC (WePure Biotech XP tC18, 7 μm, 150 mm×30 mm; 7 min gradient (37-67% ACN/H₂O (with 0.2% FA)) at 25 mL/min) to afford the title compound (2S,4S)-4-((5-((1-Ethyl-1H-pyrazol-5-yl)ethynyl)-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid as an off-white powder (46.5 mg, 41%). LCMS (ESI): mass calcd for C₃₇H₃₉N₇O₆, 677.3; m/z found, 678.4 [M+H]⁺. ¹H NMR (400 MHz, DMSO-d₆) δ 8.12-8.05 (m, 2H), 7.74-7.61 (m, 2H), 7.54-7.49 (m, 1H), 7.49-7.43 (m, 1H), 7.36-7.31 (m, 1H), 6.61-6.54 (m, 1H), 5.74-5.65 (m, 1H), 5.16-5.04 (m, 1H), 4.69-4.63 (m, 1H), 4.60-4.51 (m, 2H), 4.39-4.27 (m, 4H), 4.26-4.17 (m, 1H), 4.15-4.07 (m, 1H), 3.81-3.74 (m, 1H), 3.72-3.61 (m, 1H), 3.33-3.25 (m, 1H), 3.01-2.95 (m, 2H), 2.79-2.71 (m, 1H), 2.61-2.55 (m, 1H), 1.48-1.37 (m, 3H), 1.36-1.29 (m, 6H), 0.91-0.81 (m, 3H).

Examples 109, 111-114, 116-117, and 121-123 and 129 in Table 14 were prepared in a manner analogous to Example 110, using (2S,4S)-4-((5-Ethynyl-3-((S)-9-methyl-2,5-dioxa-8-azaspiro[3.5]nonan-8-yl)pyridin-2-yl)oxy)-1-(2-isopropylbenzofuro[3,2-c]pyrimidin-4-yl)pyrrolidine-2-carboxylic acid (intermediate 34) or (2S,4S)-1-(2-ethylbenzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid (intermediate 43) and the corresponding halide.

TABLE 14

| Ex | Structure | Halide | MS and ¹H-NMR |
|---|---|---|---|
| 109 | 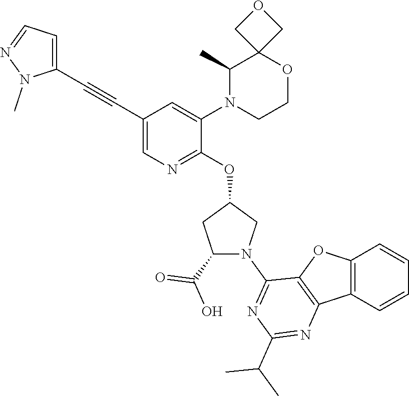 [SMILES: CC(C)c1nc(N2C[C@@H](Oc3ncc(C#Cc4ccnn4C)cc3N3CCOC4(COC4)[C@@H]3C)C[C@H]2C(=O)O)c2oc3ccccc3c2n1] | 5-bromo-1-methyl-1H- pyrazole | LCMS (ESI): mass calcd for C₃₆H₃₇N₇O₆, 663.3; m/z found, 664.3 [M + H]⁺. ¹H NMR (400 MHz, DMSO-d₆) δ 8.11 − 8.07 (m, 2H), 7.74 − 7.69 (m, 1H), 7.68 − 7.62 (m, 1H), 7.50 − 7.48 (m, 1H), 7.47 − 7.42 (m, 1H), 7.35 (d, J = 1.2 Hz, 1H), 6.58 (d, J = 1.6 Hz, 1H), 5.73 − 5.64 (m, 1H), 5.20 − 5.02 (m, 1H), 4.69 − 4.63 (m, 1H), 4.63 − 4.51 (m, 2H), 4.39 − 4.34 (m, 1H), 4.34 − 4.28 (m, 1H), 4.28 − 4.15 (m, 1H), 4.14 − 4.09 (m, 1H), 3.95 (s, 3H), 3.81 − 3.75 (m, 1H), 3.71 − 3.61 (m, 1H), 3.33 − 3.25 (m, 2H), 2.99 − 2.96 (m, 1H), 2.78 − 2.71 (m, 1H), 2.60 − 2.53 (m, 1H), 1.36 − 1.28 (m, 6H), 0.85 (d, J = 6.4 Hz, 3H). |
| | (2S,4S)-1-(2-Isopropylbenzofuro[3,2- | | |
| | d]pyrimidin-4-yl)-4-((5-((1-methyl-1H- | | |
| | pyrazol-5-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 111 | 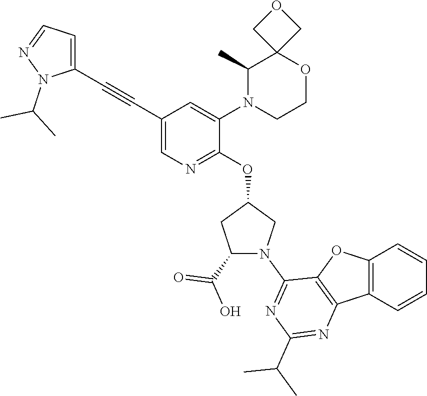 [SMILES: CC(C)c1nc(N2C[C@@H](Oc3ncc(C#Cc4ccnn4C(C)C)cc3N3CCOC4(COC4)[C@@H]3C)C[C@H]2C(=O)O)c2oc3ccccc3c2n1] | 5-bromo-1-isopropyl- 1H-pyrazole | LCMS (ESI): mass calcd for C₃₈H₄₁N₇O₆, 691.3; m/z found, 692.3 [M + H]⁺. ¹H NMR (400 MHz, DMSO-d₆) δ 8.11 − 8.09 (m, 1H), 8.09 − 8.08 (m, 1H), 7.74 − 7.69 (m, 1H), 7.67 (d, J = 7.6 Hz, 1H), 7.53 (d, J = 1.6 Hz, 1H), 7.50 − 7.44 (m, 1H), 7.33 (d, J = 2.0 Hz, 1H), 6.58 (d, J = 1.6 Hz, 1H), 5.72 − 5.66 (m, 1H), 5.16 − 5.05 (m, 1H), 4.93 − 4.84 (m, 1H), 4.67 (d, J = 6.4 Hz, 1H), 4.63 − 4.48 (m, 2H), 4.37 (d, J = 6.8 Hz, 1H), 4.35 − 4.28 (m, 1H), 4.28 − 4.18 (m, 1H), 4.12 (d, J = 6.8 Hz, 1H), 3.82 − 3.73 (m, 1H), 3.72 − 3.62 (m, 1H), 3.34 − 3.27 (m, 2H), 2.98 − 2.95 (m, 1H), 2.78 − 2.72 (m, 1H), 2.59 − 2.54 (m, 1H), 1.48 (d, J = 6.8 Hz, 6H), 1.35 − 1.30 (m, 6H), 0.85 (d, J = 6.8 Hz, 3H). |
| | (2S,4S)-4-((5-((1-Isopropyl-1H-pyrazol- | | |
| | 5-yl)ethynyl)-3-((S)-9-methyl-2,5-dioxa- | | |
| | 8-azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 112 | 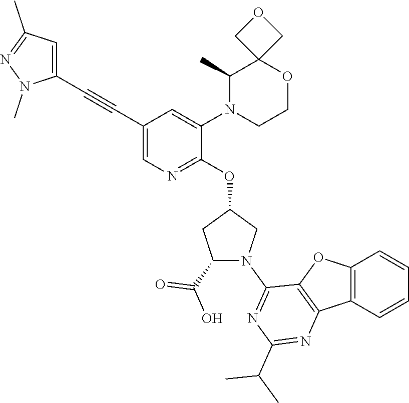 [SMILES: Cc1cc(C#Cc2cnc(O[C@H]3C[C@@H](C(=O)O)N(c4nc(C(C)C)nc5c4oc4ccccc45)C3)c(N3CCOC4(COC4)[C@@H]3C)c2)n(C)n1] | 5-bromo-1,3-dimethyl- 1H-pyrazole | LCMS (ESI): mass calcd for C₃₇H₃₉N₇O₆, 677.3; m/z found, 678.3 [M + H]⁺. ¹H NMR (400 MHz, DMSO-d₆) δ 8.11 − 8.05 (m, 2H), 7.73 − 7.69 (m, 1H), 7.68 − 7.63 (m, 1H), 7.50 − 7.44 (m, 1H), 7.35 − 7.32 (m, 1H), 6.34 (s, 1H), 5.73 − 5.66 (m, 1H), 5.22 − 5.00 (m, 1H), 4.69 − 4.63 (m, 1H), 4.61 − 4.50 (m, 2H), 4.38 − 4.34 (m, 1H), 4.33 − 4.28 (m, 1H), 4.28 − 4.17 (m, 1H), 4.15 − 4.08 (m, 1H), 3.86 (s, 3H), 3.80 − 3.74 (m, 1H), 3.70 − 3.61 (m, 1H), 3.34 − 3.25 (m, 2H), 3.01 − 2.95 (m, 1H), 2.78 − 2.70 (m, 1H), 2.61 − 2.55 (m, 1H), 2.18 (s, 3H), 1.35 − 1.30 (m, 6H), 0.84 (d, J = 6.8 Hz, 3H). |
| | (2S,4S)-4-((5-((1,3-Dimethyl-1H-pyrazol- | | |
| | 5-yl)ethynyl)-3-((S)-9-methyl-2,5-dioxa- | | |
| | 8-azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 113 | 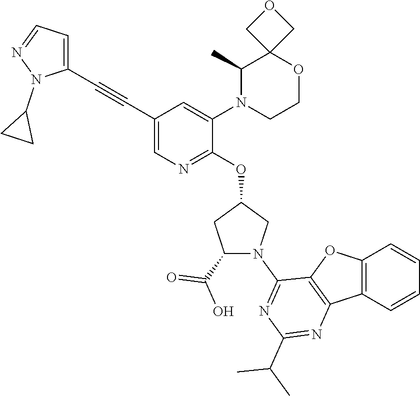 [SMILES: CC(C)c1nc(N2C[C@@H](Oc3ncc(C#Cc4ccnn4C4CC4)cc3N3CCOC4(COC4)[C@@H]3C)C[C@H]2C(=O)O)c2oc3ccccc3c2n1] | 5-bromo-1- cyclopropyl-1H- pyrazole | LCMS (ESI): mass calcd for C₃₈H₃₉N₇O₆, 689.3; m/z found, 690.3 [M + H]⁺. ¹H NMR (400 MHz, DMSO-d₆) δ 8.10 − 8.07 (m, 2H), 7.73 − 7.68 (m, 1H), 7.68 − 7.62 (m, 1H), 7.49 − 7.43 (m, 2H), 7.33 (d, J = 1.6 Hz, 1H), 6.57 (d, J = 1.6 Hz, 1H), 5.73 − 5.64 (m, 1H), 5.18 − 4.99 (m, 1H), 4.69 − 4.63 (m, 1H), 4.62 − 4.48 (m, 2H), 4.42 − 4.27 (m, 2H), .4.27 − 4.18 (m, 1H), 4.15 − 4.05 (m, 1H), 3.89 − 3.81 (m, 1H), 3.81 − 3.74 (m, 1H), 3.71 − 3.62 (m, 1H), 3.33 − 3.27 (m, 1H), 3.01 − 2.94 (m, 2H), 2.79 − 2.71 (m, 1H), 2.60 − 2.52 (m, 1H), 1.36 − 1.29 (m, 6H), 1.18 − 1.06 (m, 4H), 0.85 (d, J = 6.4 Hz, 3H). |
| | (2S,4S)-4-((5-((1-Cyclopropyl-1H- | | |
| | pyrazol-5-yl)ethynyl)-3-((S)-9-methyl- | | |
| | 2,5-dioxa-8-azaspiro[3.5]nonan-8- | | |
| | yl)pyridin-2-yl)oxy)-1-(2- | | |
| | isopropylbenzofuro[3,2-d]pyrimidin-4- | | |
| | yl)pyrrolidine-2-carboxylic acid | | |
| 114 | 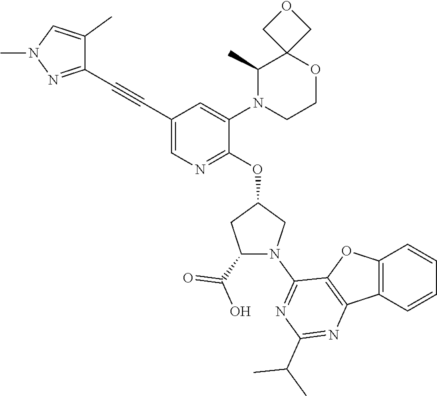 [SMILES: Cc1cn(C)nc1C#Cc1cnc(O[C@H]2C[C@@H](C(=O)O)N(c3nc(C(C)C)nc4c3oc3ccccc34)C2)c(N2CCOC3(COC3)[C@@H]2C)c1] | 3-bromo-1, 4-dimethyl- 1H-pyrazole | LCMS (ESI): mass calcd for C₃₇H₃₉N₇O₆, 677.3; m/z found, 678.3 [M + H]⁺. ¹H NMR (400 MHz, DMSO-d₆) δ 8.08 (d, J = 7.6 Hz, 1H), 8.04 − 7.98 (m, 1H), 7.74 − 7.68 (m, 1H), 7.68 − 7.61 (m, 1H), 7.54 − 7.50 (m, 1H), 7.49 − 7.43 (m, 1H), 7.29 − 7.24 (m, 1H), 5.72 − 5.63 (m, 1H), 5.19 − 5.00 (m, 1H), 4.69 − 4.63 (m, 1H), 4.62 − 4.50 (m, 2H), 4.38 − 4.34 (m, 1H), 4.34 − 4.27 (m, 1H), 4.27 − 4.16 (m, 1H), 4.14 − 4.09 (m, 1H), 3.80 (s, 3H), 3.79 − 3.74 (m, 1H), 3.70 − 3.62 (m, 1H), 3.33 − 3.28 (m, 1H), 3.01 − 2.94 (m, 2H), 2.76 − 2.71 (m, 1H), 2.60 − 2.54 (m, 1H), 2.10 (s, 3H), 1.34 − 1.30 (m, 6H), 0.85 (d, J = 6.8 Hz, 3H). |
| | (2S,4S)-4-((5-((1,4-Dimethyl-1H-pyrazol- | | |
| | 3-yl)ethynyl)-3-((S)-9-methyl-2,5-dioxa- | | |
| | 8-azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 116 | 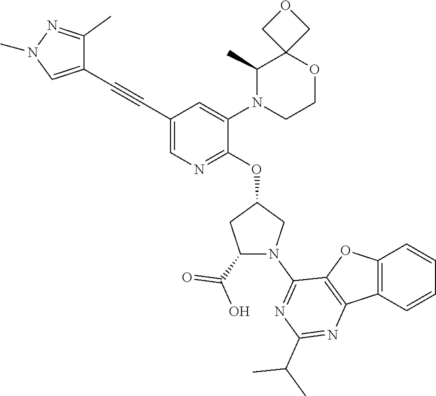 [SMILES: Cc1nn(C)cc1C#Cc1cnc(O[C@H]2C[C@@H](C(=O)O)N(c3nc(C(C)C)nc4c3oc3ccccc34)C2)c(N2CCOC3(COC3)[C@@H]2C)c1] | 4-bromo-1, 3-dimethyl- 1H-pyrazole | LCMS (ESI): mass calcd for C₃₇H₃₉N₇O₆, 677.3; m/z found, 678.3 [M + H]⁺. ¹H NMR (400 MHz, DMSO-d₆) δ 8.08 (d, J = 7.6 Hz, 1H), 7.96 − 7.93 (m, 1H), 7.85 (s, 1H), 7.73 − 7.68 (m, 1H), 7.68 − 7.62 (m, 1H), 7.49 − 7.43 (m, 1H), 7.22 − 7.20 (m, 1H), 5.69 − 5.62 (m, 1H), 5.16 − 5.01 (m, 1H), 4.69 − 4.63 (m, 1H), 4.60 − 4.48 (m, 2H), 4.39 − 4.34 (m, 1H), 4.33 − 4.27 (m, 1H), 4.25 − 4.17 (m, 1H), 4.13 − 4.08 (m, 1H), 3.78 (s, 3H), 3.77 − 3.73 (m, 1H), 3.71 − 3.55 (m, 2H), 3.31 − 3.29 (m, 1H), 2.99 − 2.97 (m, 1H), 2.74 − 2.65 (m, 2H), 2.24 (s, 3H), 1.34 − 1.29 (m, 6H), 0.83 (d, J = 6.8 Hz, 3H). |
| | (2S,4S)-4-((5-((1,3-Dimethyl-1H-pyrazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 117 | 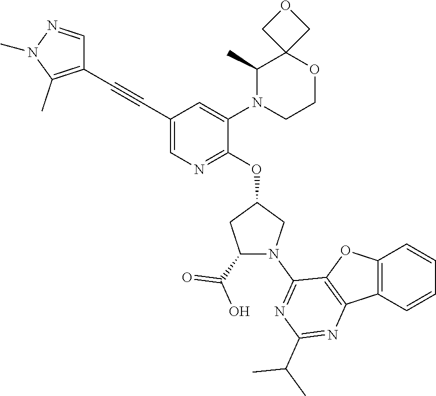 [SMILES: Cc1c(C#Cc2cnc(O[C@H]3C[C@@H](C(=O)O)N(c4nc(C(C)C)nc5c4oc4ccccc45)C3)c(N3CCOC4(COC4)[C@@H]3C)c2)cnn1C] | 4-bromo-1,5-dimethyl- 1H-pyrazole | LCMS (ESI): mass calcd for C₃₇H₃₉N₇O₆, 677.3; m/z found, 678.3 [M + H]⁺. ¹H NMR (400 MHz, DMSO-d₆) δ 8.08 (d, J = 8.0 Hz, 1H), 7.96 (s, 1H), 7.74 − 7.68 (m, 1H), 7.67 − 7.61 (m, 1H), 7.53 (s, 1H), 7.49 − 7.42 (m, 1H), 7.23 (s, 1H), 5.70 − 5.59 (m, 1H), 5.16 − 5.00 (m, 1H), 4.71 − 4.62 (m, 1H), 4.62 − 4.45 (m, 2H), 4.40 − 4.34 (m, 1H), 4.34 − 4.25 (m, 1H), 4.25 − 4.13 (m, 1H), 4.13 − 4.07 (m, 1H), 3.83 − 3.70 (m, 4H), 3.70 − 3.60 (m, 1H), 3.34 − 3.26 (m, 1H), 3.01 − 2.97 (m, 2H), 2.75 − 2.69 (m, 1H), 2.59 − 2.52 (m, 1H), 2.37 (s, 3H), 1.35 − 1.29 (m, 6H), 0.84 (d, J = 6.4 Hz, 3H). |
| | (2S,4S)-4-((5-((1,5-Dimethyl-1H-pyrazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 121 | 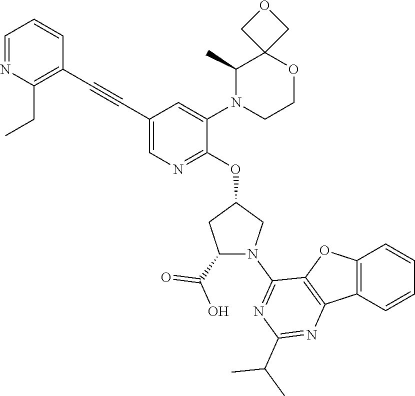 [SMILES: CCc1ncccc1C#Cc1cnc(O[C@H]2C[C@@H](C(=O)O)N(c3nc(C(C)C)nc4c3oc3ccccc34)C2)c(N2CCOC3(COC3)[C@@H]2C)c1] | 3-bromo-2- ethylpyridine | LCMS (ESI): mass calcd for C₃₉H₄₀N₆O₆, 688.3; m/z found, 689.4 [M + H]⁺. ¹H NMR (400 MHz, DMSO-d₆) δ 8.52 − 8.47 (m, 1H), 8.06 − 8.01 (m, 2H), 7.90 − 7.84 (m, 1H), 7.67 − 7.61 (m, 1H), 7.61 − 7.55 (m, 1H), 7.44 − 7.39 (m, 1H), 7.29 − 7.24 (m, 2H), 5.62 − 5.52 (m, 1H), 4.99 − 4.92 (m, 1H), 4.74 − 4.69 (m, 1H), 4.58 − 4.51 (m, 2H), 4.50 − 4.47 (m, 1H), 4.41 − 4.35 (m, 1H), 4.13 − 4.08 (m, 1H), 4.06 − 3.90 (m, 1H), 3.80 − 3.75 (m, 1H), 3.73 − 3.65 (m, 1H), 3.28 − 3.26 (m, 1H), 3.05 − 3.02 (m, 3H), 2.87 − 2.82 (m, 1H), 2.81 − 2.73 (m, 2H), 1.34 − 1.29 (m, 9H), 0.84 (d, J = 6.4 Hz, 3H). |
| | (2S,4S)-4-((5-((2-Ethylpyridin-3- | | |
| | yl)ethynyl)-3-((S)-9-methyl-2,5-dioxa-8- | | |
| | azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 122 | 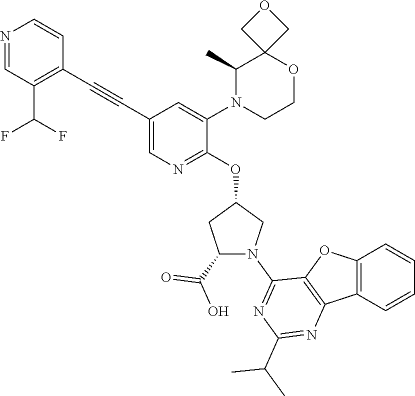 [SMILES: CC(C)c1nc(N2C[C@@H](Oc3ncc(C#Cc4ccncc4C(F)F)cc3N3CCOC4(COC4)[C@@H]3C)C[C@H]2C(=O)O)c2oc3ccccc3c2n1] | 3-bromo-2- (difluoromethyl) pyridine | LCMS (ESI): mass calcd for C₃₈H₃₆F₂N₆O₆, 710.3; m/z found, 711.3 [M + H]⁺. ¹H NMR (400 MHz, DMSO-d₆) δ 8.70 − 8.67 (m, 1H), 8.14 − 8.11 (m, 1H), 8.10 (d, J = 2.0 Hz, 1H), 8.10 − 8.07 (m, 1H), 7.73 − 7.69 (m, 1H), 7.68 − 7.65 (m, 1H), 7.65 − 7.61 (m, 1H), 7.50 − 7.43 (m, 1H), 7.37 − 7.09 (m, 2H), 5.73 − 5.67 (m, 1H), 5.16 − 5.06 (m, 1H), 4.69 − 4.63 (m, 1H), 4.61 − 4.50 (m, 2H), 4.38 − 4.35 (m, 1H), 4.34 − 4.28 (m, 1H), 4.28 − 4.18 (m, 1H), 4.15 − 4.08 (m, 1H), 3.82 − 3.74 (m, 1H), 3.72 − 3.62 (m, 1H), 3.34 − 3.23 (m, 1H), 3.03 − 2.96 (m, 1H), 2.80 − 2.73 (m, 1H), 2.61 − 2.51 (m, 2H), 1.36 − 1.29 (m, 6H), 0.86 (d, J = 6.8 Hz, 3H). |
| | (2S,4S)-4-((5-((2- | | |
| | (Difluoromethyl)pyridin-3-yl)ethynyl)-3- | | |
| | ((S)-9-methyl-2,5-dioxa-8- | | |
| | azaspiro[3.5]nonan-8-yl)pyridin-2- | | |
| | yl)oxy)-1-(2-isopropylbenzofuro[3,2- | | |
| | d]pyrimidin-4-yl)pyrrolidine-2-carboxylic | | |
| | acid | | |
| 123 | 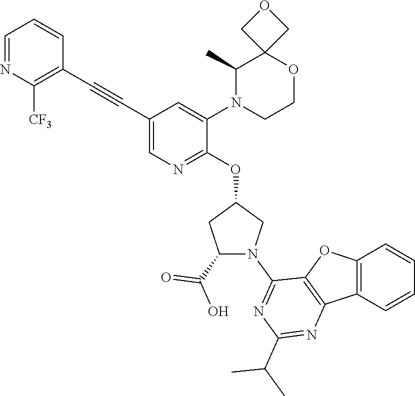 [SMILES: CC(C)c1nc(N2C[C@@H](Oc3ncc(C#Cc4cccnc4C(F)(F)F)cc3N3CCOC4(COC4)[C@@H]3C)C[C@H]2C(=O)O)c2oc3ccccc3c2n1] | 3-bromo-2- (trifluoromethyl) pyridine | LCMS (ESI): mass calcd for C₃₈H₃₅F₃N₆O₆, 728.3; m/z found, 729.3 [M + H]⁺. ¹H NMR (400 MHz, DMSO-d₆) δ 12.55 (br s, 1H), 8.73 (d, J = 4.8 Hz, 1H), 8.25 (d, J = 8.0 Hz, 1H), 8.11 − 8.05 (m, 2H), 7.80 − 7.75 (m, 1H), 7.74 − 7.69 (m, 1H), 7.68 − 7.63 (m, 1H), 7.50 − 7.44 (m, 1H), 7.31 − 7.28 (m, 1H), 5.78 − 5.61 (m, 1H), 5.20 − 5.03 (m, 1H), 4.70 − 4.63 (m, 1H), 4.60 − 4.52 (m, 2H), 4.39 − 4.31 (m, 2H), 4.30 − 4.19 (m, 1H), 4.14 − 4.09 (m, 1H), 3.82 − 3.75 (m, 1H), 3.71 − 3.63 (m, 1H), 3.33 − 3.26 (m, 1H), 3.02 − 2.97 (m, 1H), 2.79 − 2.72 (m, 1H), 2.62 − 2.52 (m, 2H), 1.33 (d, J = 6.8 Hz, 6H), 0.86 (d, J = 6.4 Hz, 3H); |
| | (2S,4S)-1-(2-Isopropylbenzofuro[3,2- | | |
| | d]pyrimidin-4-yl)-4-((3-((S)-9-methyl- | | |
| | 2,5-dioxa-8-azaspiro[3.5]nonan-8-yl)-5- | | |
| | ((2-(trifluoromethyl)pyridin-3- | | |
| | yl)ethynyl)pyridin-2-yl)oxy)pyrrolidine- | | |
| | 2-carboxylic acid | | |
| 129 | 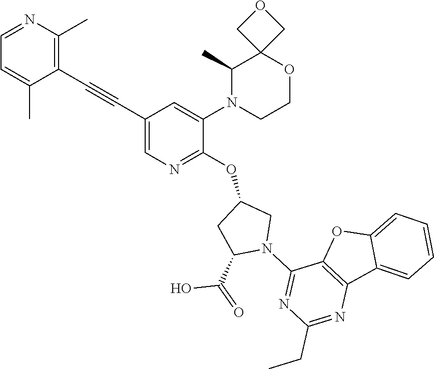 [SMILES: CCc1nc(N2C[C@@H](Oc3ncc(C#Cc4c(C)ccnc4C)cc3N3CCOC4(COC4)[C@@H]3C)C[C@H]2C(=O)O)c2oc3ccccc3c2n1] | 3-bromo-2,4- dimethylpyridine; Intermediate 43 was used and the reaction mixture was heated in a microwave reactor at 80º C. for 1.5 h | LCMS (ESI): mass calcd for C₃₈H₃₈N₆O₆ 674.3 m/z found, 675.5 [M + H]⁺. ¹H NMR (400 MHz, DMSO− d₆) δ 8.24 (d, J = 5.1 Hz, 1H), 8.08 − 8.01 (m, 2H), 7.63 (dt, J = 15.4, 8.3 Hz, 2H), 7.42 (t, J = 7.4 Hz, 1H), 7.27 (d, J = 2.0 Hz, 1H), 7.13 (d, J = 5.1 Hz, 1H), 5.62 (s, 1H), 5.08 (s, 1H), 4.63 (d, J = 6.5 Hz, 1H), 4.51 (d, J = 6.6 Hz, 2H), 4.33 (d, J = 6.7 Hz, 1H), 4.27 (d, J = 6.6 Hz, 1H), 4.16 (s, 1H), 4.08 (d, J = 6.7 Hz, 1H), 3.74 (d, J = 11.3 Hz, 1H), 3.67 − 3.57 (m, 1H), 3.32 − 3.20 (m, 1H), 3.00 − 2.88 (m, 1H), 2.79 (q, J = 7.5 Hz, 2H), 2.71 (d, J = 11.9 Hz, 1H), 2.62 (s, 3H), 2.43 (s, 3H), 1.28 (t, J = 7.6 Hz, 3H), 0.81 (d, J = 6.6 Hz, 3H). |
| | (2S,4S)-4-((5-((2,4-Dimethylpyridin-3- | | |
| | yl)ethynyl)-3-((S)-9-methyl-2,5-dioxa-8- | | |
| | azaspiro[3.5]nonan-8-yl)pyridin-2- | | |
| | yl)oxy)-1-(2-ethylbenzofuro[3,2- | | |
| | d]pyrimidin-4-yl)pyrrolidine-2-carboxylic | | |
| | acid; | | |

Examples 115, 119, 124-125 in Table 15 were prepared in a manner analogous to Example 1, using (2S,4S)-4-((5-bromo-3-((S)-9-methyl-2,5-dioxa-8-azaspiro[3.5]nonan-8-yl)pyridin-2-yl)oxy)-1-(2-isopropylbenzofuro[3,2-d]pyrimidin-4-yl)pyrrolidine-2-carboxylic acid (intermediate 29) and the corresponding alkyne.

TABLE 15

| Ex # | Structure | alkyne | MS and 1H-NMR |
|---|---|---|---|
| 115 | 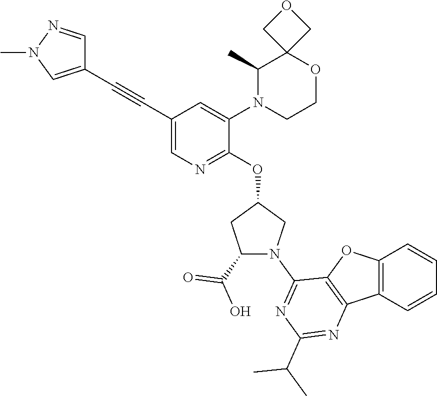 [SMILES: CC(C)c1nc(N2C[C@@H](Oc3ncc(C#Cc4cnn(C)c4)cc3N3CCOC4(COC4)[C@@H]3C)C[C@H]2C(=O)O)c2oc3ccccc3c2n1] | 4-ethynyl-1-methyl-1H- pyrazole | LCMS (ESI): mass calcd for C₃₆H₃₇N₇O₆, 663.3; m/z found, 664.3 [M + H]⁺. ¹H NMR (400 MHz, DMSO-d₆) δ 8.08 (d, J = 7.6 Hz, 1H), 7.98 (s, 1H), 7.95 − 7.93 (m, 1H), 7.72 − 7.68 (m, 1H), 7.67 − 7.64 (m, 1H), 7.63 (s, 1H), 7.48 − 7.43 (m, 1H), 7.23 − 7.20 (m, 1H), 5.68 − 5.63 (m, 1H), 5.14 − 5.03 (m, 1H), 4.67 − 4.63 (m, 1H), 4.60 − 4.49 (m, 2H), 4.37 − 4.33 (m, 1H), 4.31 − 4.25 (m, 1H), 4.24 − 4.15 (m, 1H), 4.13 − 4.08 (m, 1H), 3.86 (s, 3H), 3.79 − 3.72 (m, 1H), 3.70 − 3.60 (m, 1H), 3.30 − 3.26 (m, 2H), 2.99 − 2.97 (m, 2H), 2.73 − 2.68 (m, 1H), 1.34 − 1.27 (m, 6H), 0.83 (d, J = 6.8 Hz, 3H). |
| | (2S,4S)-1-(2-Isopropylbenzofuro[3,2- | | |
| | d]pyrimidin-4-yl)-4-((5-((1-methyl-1H- | | |
| | pyrazol-4-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 119 | 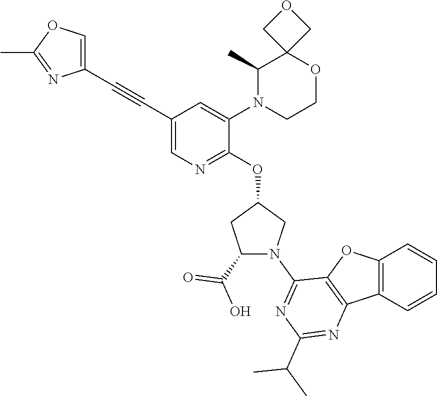 [SMILES: Cc1nc(C#Cc2cnc(O[C@H]3C[C@@H](C(=O)O)N(c4nc(C(C)C)nc5c4oc4ccccc45)C3)c(N3CCOC4(COC4)[C@@H]3C)c2)co1] | 4-ethynyl-2- methyloxazole | LCMS (ESI): mass calcd for C₃₆H₃₆N₆O₇, 664.3; m/z found, 665.3 [M + H]⁺. ¹H NMR (400 MHz, DMSO-d₆) δ 8.28 (s, 1H), 8.09 (d, J = 7.6 Hz, 1H), 8.01 (d, J = 2.0 Hz, 1H), 7.73 − 7.61 (m, 2H), 7.49 − 7.43 (m, 1H), 7.27 (d, J = 2.0 Hz, 1H), 5.70 − 5.64 (m, 1H), 5.15 − 5.02 (m, 1H), 4.67 − 4.62 (m, 1H), 4.60 − 4.49 (m, 2H), 4.38 − 4.33 (m, 1H), 4.32 − 4.27 (m, 1H), 4.26 − 4.18 (m, 1H), 4.13 − 4.09 (m, 1H), 3.79 − 3.74 (m, 1H), 3.70 − 3.60 (m, 2H), 3.32 − 3.31 (m, 1H), 2.97 − 2.95 (m, 1H), 2.76 − 2.68 (m, 2H), 2.43 (s, 3H), 1.33 − 1.30 (m, 6H), 0.84 (d, J = 6.4 Hz, 3H). |
| | (2S,4S)-1-(2-Isopropylbenzofuro[3,2- | | |
| | d]pyrimidin-4-yl)-4-((3-((S)-9-methyl- | | |
| | 2,5-dioxa-8-azaspiro[3.5]nonan-8-yl)-5- | | |
| | ((2-methyloxazol-4-yl)ethynyl)pyridin-2- | | |
| | yl)oxy)pyrrolidine-2-carboxylic acid | | |
| 124 | 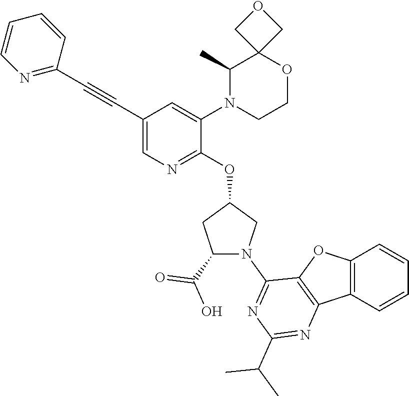 [SMILES: CC(C)c1nc(N2C[C@@H](Oc3ncc(C#Cc4ccccn4)cc3N3CCOC4(COC4)[C@@H]3C)C[C@H]2C(=O)O)c2oc3ccccc3c2n1] | 2-ethynylpyridine | LCMS (ESI): mass calcd for C₃₇H₃₆N₆O₆, 660.3; m/z found, 661.3 [M + H]⁺. ¹H NMR (400 MHz, DMSO-d₆) δ 8.61 (d, J = 4.4 Hz, 1H), 8.12 − 8.05 (m, 2H), 7.87 − 7.81 (m, 1H), 7.73 − 7.69 (m, 1H), 7.68 − 7.61 (m, 2H), 7.49 − 7.44 (m, 1H), 7.42 − 7.37 (m, 1H), 7.36 − 7.34 (m, 1H), 5.73 − 5.66 (m, 1H), 5.18 − 5.02 (m, 1H), 4.71 − 4.64 (m, 1H), 4.61 − 4.51 (m, 2H), 4.40 − 4.28 (m, 2H), 4.27 − 4.16 (m, 1H), 4.15 − 4.07 (m, 1H), 3.82 − 3.75 (m, 1H), 3.73 − 3.63 (m, 1H), 3.34 − 3.26 (m, 2H), 3.00 − 2.97 (m, 1H), 2.78 − 2.72 (m, 1H), 2.59 − 2.55 (m, 1H), 1.36 − 1.28 (m, 6H), 0.85 (d, J = 6.4 Hz, 3H). |
| | (2S,4S)-1-(2-Isopropylbenzofuro[3,2- | | |
| | d]pyrimidin-4-yl)-4-((3-((S)-9-methyl- | | |
| | 2,5-dioxa-8-azaspiro[3.5]nonan-8-yl)-5- | | |
| | (pyridin-2-ylethynyl)pyridin-2- | | |
| | yl)oxy)pyrrolidine-2-carboxylic acid | | |
| 125 | 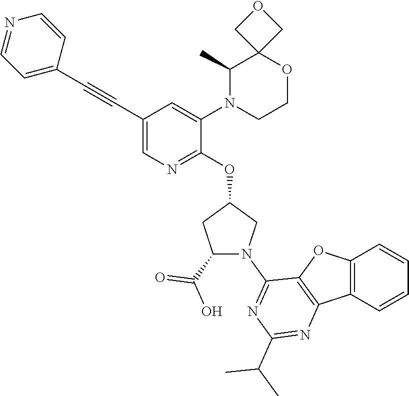 [SMILES: CC(C)c1nc(N2C[C@@H](Oc3ncc(C#Cc4ccncc4)cc3N3CCOC4(COC4)[C@@H]3C)C[C@H]2C(=O)O)c2oc3ccccc3c2n1] | 4-ethynylpyridine | LCMS (ESI): mass calcd for C₃₇H₃₆N₆O₆, 660.3; m/z found, 661.3 [M + H]⁺. ¹H NMR (400 MHz, DMSO-d₆) δ 8.67 − 8.59 (m, 2H), 8.11 − 8.06 (m, 2H), 7.73 − 7.67 (m, 1H), 7.67 − 7.61 (m, 1H), 7.52 − 7.48 (m, 2H), 7.48 − 7.44 (m, 1H), 7.36 (d, J = 1.6 Hz, 1H), 5.73 − 5.60 (m, 1H), 5.19 − 4.99 (m, 1H), 4.70 − 4.63 (m, 1H), 4.63 − 4.46 (m, 2H), 4.39 − 4.35 (m, 1H), 4.34 − 4.28 (m, 1H), 4.27 − 4.16 (m, 1H), 4.15 − 4.09 (m, 1H), 3.81 − 3.73 (m, 1H), 3.71 − 3.62 (m, 1H), 3.33 − 3.29 (m, 1H), 3.10 − 3.08 (m, 1H), 2.99 − 2.94 (m, 1H), 2.78 − 2.72 (m, 1H), 2.60 − 2.53 (m, 1H), 1.35 − 1.28 (m, 6H), 0.85 (d, J = 6.8 Hz, 3H). |
| | (2S,4S)-1-(2-Isopropylbenzofuro[3,2- | | |
| | d]pyrimidin-4-yl)-4-((3-((S)-9-methyl- | | |
| | 2,5-dioxa-8-azaspiro[3.5]nonan-8-yl)-5- | | |
| | (pyridin-4-ylethynyl)pyridin-2- | | |
| | yl)oxy)pyrrolidine-2-carboxylic acid | | |

Examples 126-128, 130-135, 138-145, 149, 151-162, 165-166, 169, 172-181, 183-187, and 191-194 in Table 16 were prepared in a manner analogous to Example 73, using (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-pyrrolidine-2-carboxylic acid Intermediate 12) and the corresponding alkyne.

TABLE 16

| Ex # | Structure | Alkyne | MS |
|---|---|---|---|
| 126 | 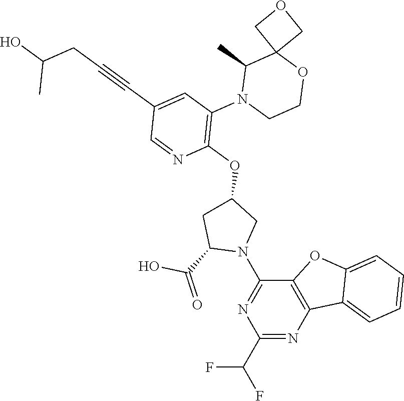 [SMILES: CC(O)CC#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-(2-(Difluoromethyl)benzofuro[3,2- d]pyrimidin-4-yl)-4-((5-(4-hydroxypent-1-yn-1-yl)-3- ((S)-9-methyl-2,5-dioxa-8-azaspiro[3.5]nonan-8- yl)pyridin-2-yl)oxy)pyrrolidine-2-carboxylic acid; | Pent-4-yn-2-ol | mass calcd for C₃₃H₃₃F₂N₅O₇ 649.23 m/z found 650.30 [M + H]⁺ |
| 127 | 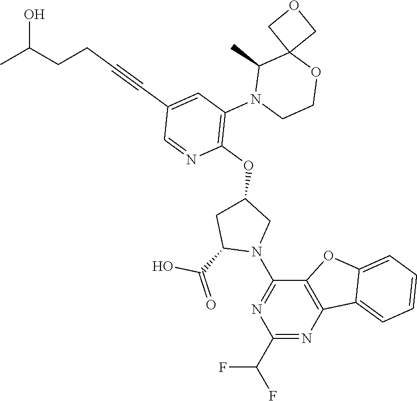 [SMILES: CC(O)CCC#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-(2-(Difluoromethyl)benzofuro[3,2- d]pyrimidin-4-yl)-4-((5-(5-hydroxyhex-1-yn-1-yl)-3- ((S)-9-methyl-2,5-dioxa-8-azaspiro[3.5]nonan-8- yl)pyridin-2-yl)oxy)pyrrolidine-2-carboxylic acid; | Hex-5-yn-2-ol | mass calcd for C₃₄H₃₅F₂N₅O₇ 663.25 m/z found 664.3 [M + H]⁺ |
| 128 | 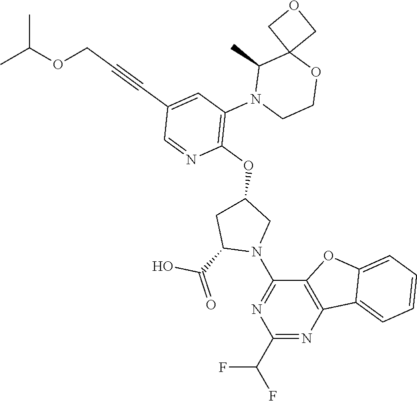 [SMILES: CC(C)OCC#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-(2-(Difluoromethyl)benzofuro[3,2- d]pyrimidin-4-yl)-4-((5-(3-isopropoxyprop-1-yn-1- yl)-3-((S)-9-methyl-2,5-dioxa-8-azaspiro[3.5]nonan- 8-yl)pyridin-2-yl)oxy)pyrrolidine-2-carboxylic acid; | 3-Isopropoxyprop-1-yne | mass calcd for C₃₄H₃₅F₂N₅O₇ 663.25 m/z found 664.50 [M + H]⁺ |
| 130 | 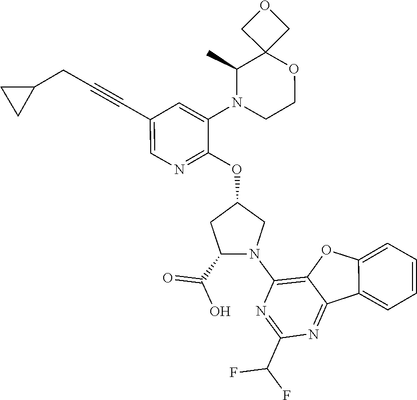 [SMILES: C[C@@H]1N(c2cc(C#CCC3CC3)cnc2O[C@H]2C[C@@H](C(=O)O)N(c3nc(C(F)F)nc4c3oc3ccccc34)C2)CCOC12COC2] (2S,4S)-4-((5-(3-Cyclopropylprop-1-yn-1-yl)-3-((S)-9- methyl-2,5-dioxa-8-azaspiro[3.5]nonan-8- yl)pyridin-2-yl)oxy)-1-(2- (difluoromethyl)benzofuro[3,2-d]pyrimidin-4- yl)pyrrolidine-2-carboxylic acid | (prop-2-yn-1- yl)cyclopropane | mass calcd for C₃₄H₃₃F₂N₅O₆ 645.24 m/z found 646.4 [M + H]⁺ |
| 131 | 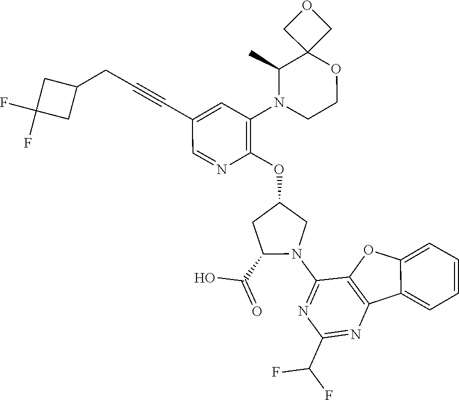 [SMILES: C[C@@H]1N(c2cc(C#CCC3CC(F)(F)C3)cnc2O[C@H]2C[C@@H](C(=O)O)N(c3nc(C(F)F)nc4c3oc3ccccc34)C2)CCOC12COC2] (2S,4S)-4-((5-(3-(3,3-Difluorocyclobutyl)prop-1-yn- 1-yl)-3-((S)-9-methyl-2,5-dioxa-8- azaspiro[3.5]nonan-8-yl)pyridin-2-yl)oxy)-1-(2- (difluoromethyl)benzofuro[3,2-d]pyrimidin-4- yl)pyrrolidine-2-carboxylic acid; | 1,1-difluoro-3-(prop-2- yn-1-yl)cyclobutane | mass calcd for C₃₅H₃₃F₄N₅O₆ 695.24 m/z found 696.30 [M + H]⁺ |
| 132 | 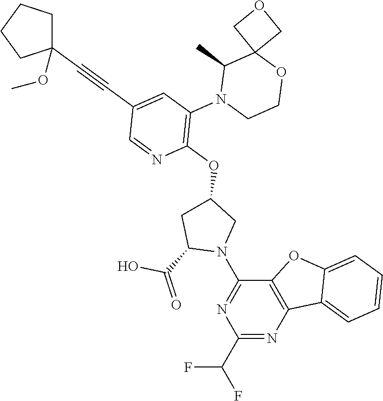 [SMILES: COC1(C#Cc2cnc(O[C@H]3C[C@@H](C(=O)O)N(c4nc(C(F)F)nc5c4oc4ccccc45)C3)c(N3CCOC4(COC4)[C@@H]3C)c2)CCCC1] (2S,4S)-1-(2-(Difluoromethyl)benzofuro[3,2- d]pyrimidin-4-yl)-4-((5-((1- methoxycyclopentyl)ethynyl)-3-((S)-9-methyl-2,5- dioxa-8-azaspiro[3.5]nonan-8-yl)pyridin-2- yl)oxy)pyrrolidine-2-carboxylic acid; | 1-ethynyl-1- methoxycyclopentane | mass calcd for C36H37F2N5O7 689.27 m/z found 690.50 [M + H]⁺ |
| 133 | 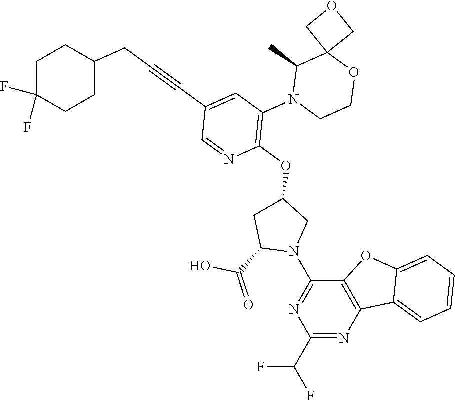 [SMILES: C[C@@H]1N(c2cc(C#CCC3CCC(F)(F)CC3)cnc2O[C@H]2C[C@@H](C(=O)O)N(c3nc(C(F)F)nc4c3oc3ccccc34)C2)CCOC12COC2] (2S,4S)-4-((5-(3-(4,4-Difluorocyclohexyl)prop-1-yn- 1-yl)-3-((S)-9-methyl-2,5-dioxa-8- azaspiro[3.5]nonan-8-yl)pyridin-2-yl)oxy)-1-(2- (difluoromethyl)benzofuro[3,2-d]pyrimidin-4- yl)pyrrolidine-2-carboxylic acid; | 1,1-difluoro-4-(prop-2- yn-1-yl)cyclohexane | mass calcd for C₃₇H₃₇F₄N₅O₆ 723.27 m/z found 724.40 [M + H]⁺ |
| 134 | 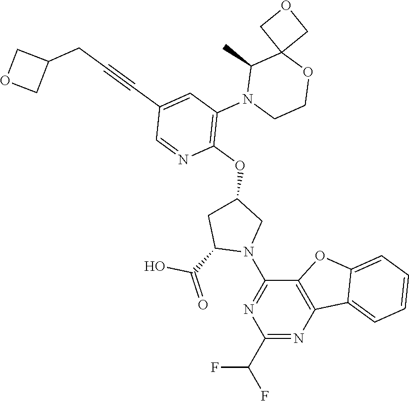 [SMILES: C[C@@H]1N(c2cc(C#CCC3COC3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(oxetan-3-yl)prop-1- yn-1-yl)pyridin-2-yl)oxy)pyrrolidine-2-carboxylic acid; | 3-(prop-2-yn-1- yl)oxetane | mass calcd for C₃₄H₃₃F₂N₅O₇ 661.23 m/z found 662.30 [M + H]⁺ |
| 135 | 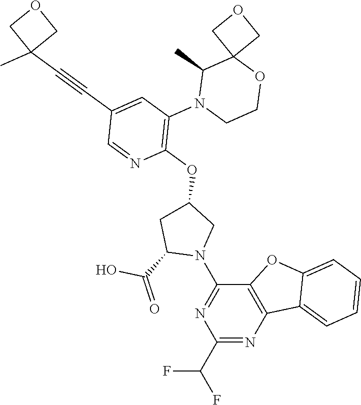 [SMILES: C[C@@H]1N(c2cc(C#CC3(C)COC3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(3-methyloxetan-3- yl)ethynyl]-2-pyridyl]oxy]pyrrolidine-2-carboxylic acid; | 3-ethynyl-3- methyloxetane | mass calcd for C₃₄H₃F₂N₅O₇ 661.23 m/z found 662.40 [M + H]⁺ |
| 138 | 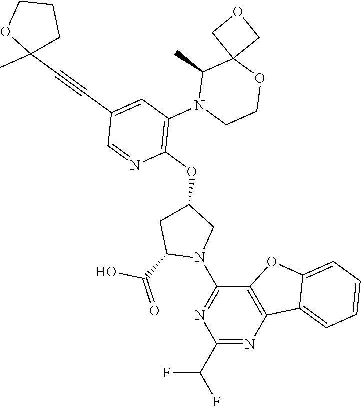 [SMILES: C[C@@H]1N(c2cc(C#CC3(C)CCCO3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-((2- methyltetrahydrofuran-2-yl)ethynyl)pyridin-2- yl)oxy)pyrrolidine-2-carboxylic acid; | 2-ethynyl-2- methyltetrahydrofuran | mass calcd for C₃₅H₃₅F₂N₅O₇ 675.25 m/z found 676.40 [M + H]⁺ |
| 139 | 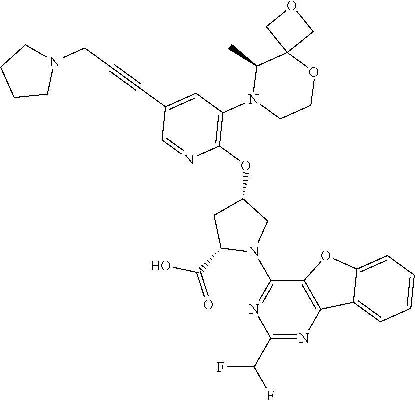 [SMILES: C[C@@H]1N(c2cc(C#CCN3CCCC3)cnc2O[C@H]2C[C@@H](C(=O)O)N(c3nc(C(F)F)nc4c3oc3ccccc34)C2)CCOC12COC2] (2S,4S)-1-(2-(difluoromethyl)benzofuro[3,2- d]pyrimidin-4-yl)-4-((3-((S)-9-methyl-2,5-dioxa-8- azaspiro[3.5]nonan-8-yl)-5-(3-(pyrrolidin-1-yl)prop- 1-yn-1-yl)pyridin-2-yl)oxy)pyrrolidine-2-carboxylic acid; | 1-(prop-2-yn-1- yl)pyrrolidine | mass calcd for C₃₅H₃₆F₂N₆O₆ 674.27 m/z found 675.40 [M + H]⁺ |
| 140 | 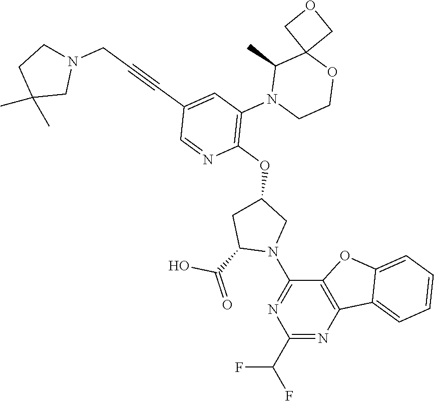 [SMILES: C[C@@H]1N(c2cc(C#CCN3CCC(C)(C)C3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((5-(3-(3,3-dimethylpyrrolidin-1- yl)prop-1-yn-1-yl)-3-((S)-9-methyl-2,5-dioxa-8- azaspiro[3.5]nonan-8-yl)pyridin-2- yl)oxy)pyrrolidine-2-carboxylic acid; | 3,3-dimethyl-1-(prop-2- yn-1-yl)pyrrolidine | mass calcd for C₃₇H₄₀F₂N₆O₆ 702.30 m/z found 703.40 [M + H]⁺ |
| 141 | 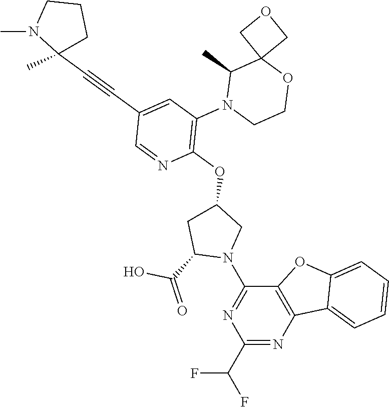 [SMILES: C[C@@H]1N(c2cc(C#C[C@]3(C)CCCN3C)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[5-[2-[(2R)-1,2- dimethylpyrrolidin-2-yl]ethynyl]-3-[(9S)-9-methyl- 2,5-dioxa-8-azaspiro[3.5]nonan-8-yl]-2- pyridyl]oxy]pyrrolidine-2-carboxylic acid; | (S)-2-ethynyl-1,2- dimethylpyrrolidine | mass calcd for C₃₆H₃₈F₂N₆O₆ 688.28 m/z found 689.40 [M + H]⁺ |
| 142 | 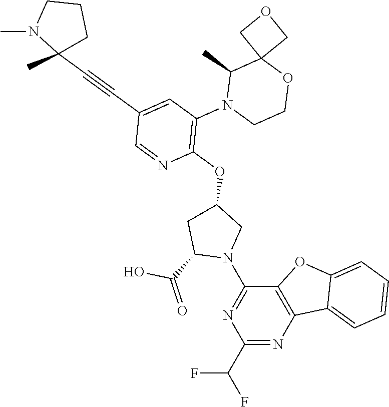 [SMILES: C[C@@H]1N(c2cc(C#C[C@@]3(C)CCCN3C)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[5-[2-[(2S)-1,2- dimethylpyrrolidin-2-yl]ethynyl]-3-[(9S)-9-methyl- 2,5-dioxa-8-azaspiro[3.5]nonan-8-yl]-2- pyridyl]oxy]pyrrolidine-2-carboxylic acid; | (R)-2-ethynyl-1,2- dimethylpyrrolidine | mass calcd for C₃₆H₃₈F₂N₆O₆ 688.28 m/z found 689.40 [M + H]⁺ |
| 143 | 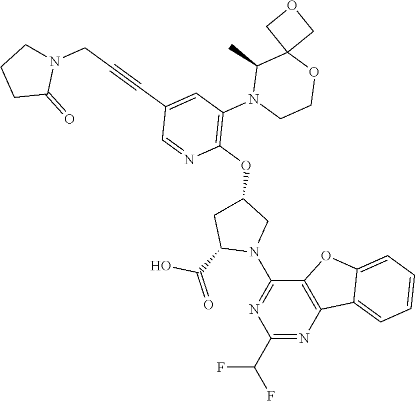 [SMILES: C[C@@H]1N(c2cc(C#CCN3CCCC3=O)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(2-oxopyrrolidin-1- yl)prop-1-yn-1-yl)pyridin-2-yl)oxy)pyrrolidine-2- carboxylic acid; | 1-(prop-2-yn-1- yl)pyrrolidine-2-one | mass calcd for C₃₅H₃₄F₂N₆O₇ 688.25 m/z found 689.30 [M + H]⁺ |
| 144 | 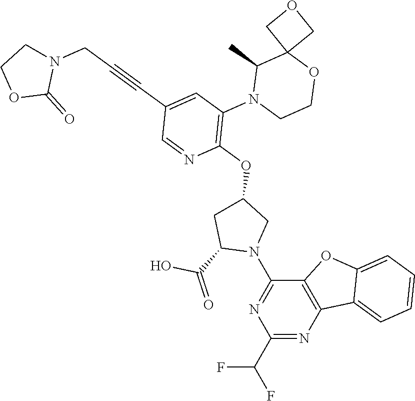 [SMILES: C[C@@H]1N(c2cc(C#CCN3CCOC3=O)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(2-oxooxazolidin-3- yl)prop-1-yn-1-yl)pyridin-2-yl)oxy)pyrrolidine-2- carboxylic acid; | 3-(prop-2-yn-1- yl)oxazolidine-2-one | mass calcd for C₃₄H₃₂F₂N₆O₈ 690.22 m/z found 691.40 [M + H]⁺ |
| 145 | 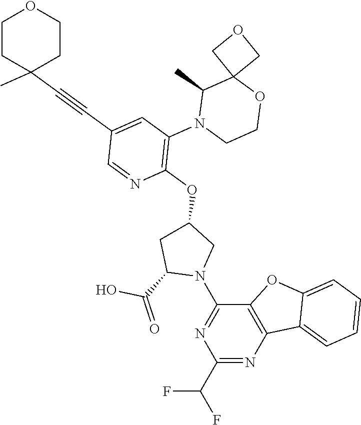 [SMILES: C[C@@H]1N(c2cc(C#CC3(C)CCOCC3)cnc2O[C@H]2C[C@@H](C(=O)O)N(c3nc(C(F)F)nc4c3oc3ccccc34)C2)CCOC12COC2] 2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-((4-methyltetrahydro- 2H-pyran-4-yl)ethynyl)pyridin-2-yl)oxy)pyrrolidine- 2-carboxylic acid; | 4-ethynyl-4- methyltetrahydro-2H- pyran | mass calcd for C₃₆H₃₇F₂N₅O₇ 689.27 m/z found 690.40 [M + H]⁺ |
| 149 | 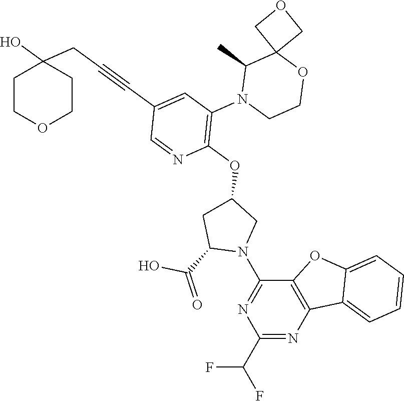 [SMILES: C[C@@H]1N(c2cc(C#CCC3(O)CCOCC3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[5-[3-(4- hydroxytetrahydropyran-4-yl)prop-1-ynyl]-3-[(9S)- 9-methyl-2,5-dioxa-8-azaspiro[3.5]nonan-8-yl]-2- pyridyl]oxy]pyrrolidine-2-carboxylic acid; | 4-(prop-2-yn-1- yl)tetrahydro-2H-pyran- 4-ol | mass calcd for C₃₆H₃₇F₂N₅O₈ 705.26 m/z found 706.40 [M + H]⁺ |
| 151 | 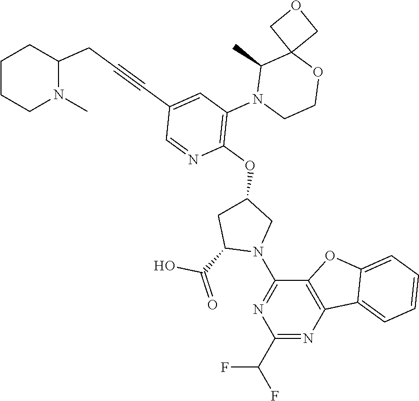 [SMILES: C[C@@H]1N(c2cc(C#CCC3CCCCN3C)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(1-methylpiperidin-2- yl)prop-1-yn-1-yl)pyridin-2-yl)oxy)pyrrolidine-2- carboxylic acid; | 1-methyl-2-(prop-2-yn- 1-yl)piperidine | mass calcd for C₃₇H₄₀F₂N₆O₆ 702.30 m/z found 703.40 [M + H]⁺ |
| 152 | 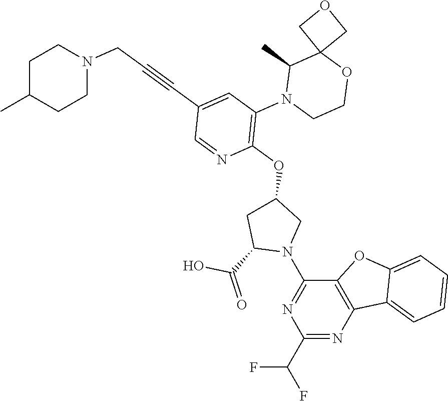 [SMILES: CC1CCN(CC#Cc2cnc(O[C@H]3C[C@@H](C(=O)O)N(c4nc(C(F)F)nc5c4oc4ccccc45)C3)c(N3CCOC4(COC4)[C@@H]3C)c2)CC1] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(4-methylpiperidin-1- yl)prop-1-yn-1-yl)pyridin-2-yl)oxy)pyrrolidine-2- carboxylic acid; | 4-methyl-1-(prop-2-yn- 1-yl)piperidine | mass calcd for C₃₇H₄₀F₂N₆O₆ 702.30 m/z found 703.40 [M + H]⁺ |
| 153 | 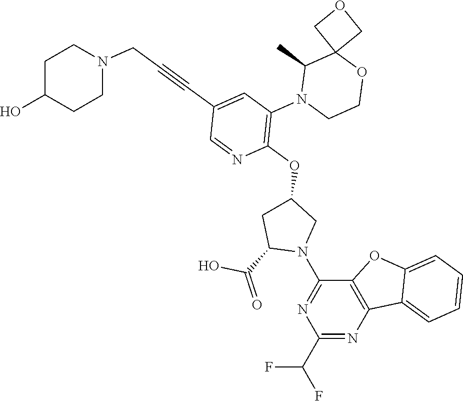 [SMILES: C[C@@H]1N(c2cc(C#CCN3CCC(O)CC3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[5-[3-(4-hydroxy-1- piperidyl)prop-1-ynyl]-3-[(9S)-9-methyl-2,5-dioxa-8- azaspiro [3.5]nonan-8-yl]-2-pyridyl]oxy]pyrrolidine- 2-carboxylic acid; | 1-(prop-2-yn-1- yl)piperidin-4-ol | mass calcd for C₃₆H₃₈F₂N₆O₇ 704.28 m/z found 705.50 [M + H]⁺ |
| 154 | 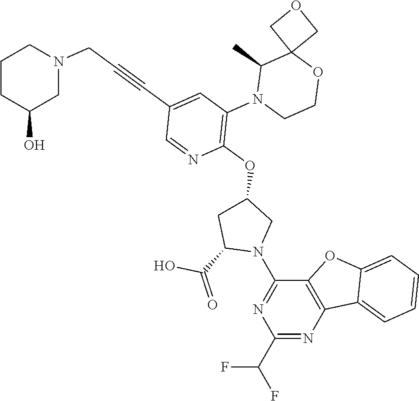 [SMILES: C[C@@H]1N(c2cc(C#CCN3CCC[C@H](O)C3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[5-[3-[(3R)-3-hydroxy-1- piperidyl]prop-1-ynyl]-3-[(9S)-9-methyl-2,5-dioxa-8- azaspiro[3.5]nonan-8-yl]-2-pyridyl]oxy]pyrrolidine- 2-carboxylic acid; | (R)-1-(prop-2-yn-1- yl)piperidin-3-ol | mass calcd for C₃₆H₃₈F₂N₆O₇ 704.28 m/z found 705.5 [M + H]⁺ |
| 155 | 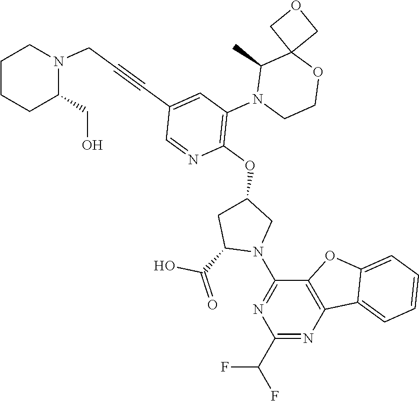 [SMILES: C[C@@H]1N(c2cc(C#CCN3CCCC[C@H]3CO)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[5-[3-[(2S)-2-(hydroxymethyl)- 1-piperidyl]prop-1-ynyl]-3-[(9S)-9-methyl-2,5-dioxa- 8-azaspiro[3.5]nonan-8-yl]-2- pyridyl]oxy]pyrrolidine-2-carboxylic acid; | (S)-(1-prop-2-yn-1- yl)piperidin-2- yl)methanol | mass calcd for C₃₇H₄₀F₂N₆O₇ 718.29 m/z found 719.50 [M + H]⁺ |
| 156 | 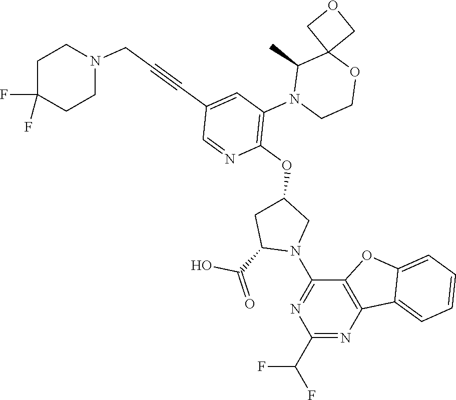 [SMILES: C[C@@H]1N(c2cc(C#CCN3CCC(F)(F)CC3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((5-(3-(4,4-difluoropiperidin-1- yl)prop-1-yn-1-yl)-3-((S)-9-methyl-2,5-dioxa-8- azaspiro[3.5]nonan-8-yl)pyridin-2- yl)oxy)pyrrolidine-2-carboxylic acid; | 4,4-difluoro-1-prop-2- yn-1-yl)piperidine | mass calcd for C₃₆H₃₆F₄N₆O₆ 724.26 m/z found 725.40 [M + H]⁺ |
| 157 | 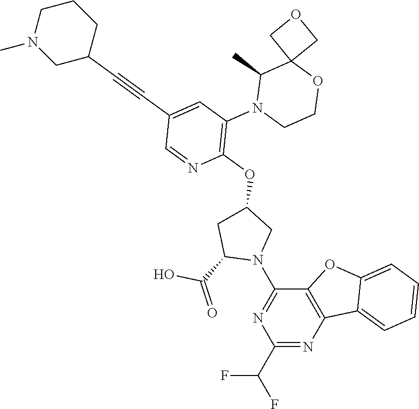 [SMILES: C[C@@H]1N(c2cc(C#CC3CCCN(C)C3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(1-methyl-3- piperidyl)ethynyl]-2-pyridyl]oxy]pyrrolidine-2- carboxylic acid; | 3-ethynyl-1- methylpiperidine | mass calcd for C₃₆H₃₈F₂N₆O₆ 688.28 m/z found 689.40 [M + H]⁺ |
| 158 | 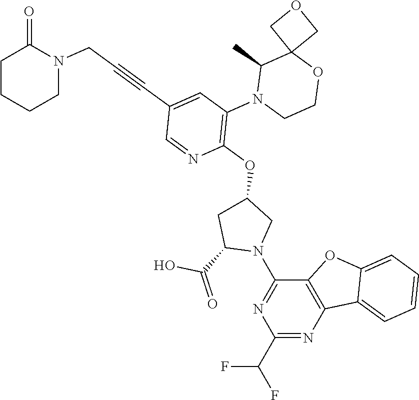 [SMILES: C[C@@H]1N(c2cc(C#CCN3CCCCC3=O)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(2-oxopiperidin-1- yl)prop-1-yn-1-yl)pyridin-2-yl)oxy)pyrrolidine-2- carboxylic acid; | 1-(prop-2-yn-1- yl)piperidin-2-one | mass calcd for C₃₆H₃₆F₂N₆O₇ 702.26 m/z found 703.30 [M + H]⁺ |
| 159 | 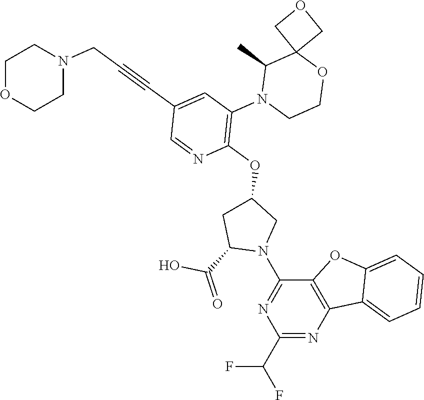 [SMILES: C[C@@H]1N(c2cc(C#CCN3CCOCC3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-morpholinoprop-1- yn-1-yl)pyridin-2-yl)oxy)pyrrolidine-2-carboxylic acid; | 4-(prop-2-yn-1- yl)morpholine | mass calcd for C₃₅H₃₆F₂N₆O₇ 690.26 m/z found 691.40 [M + H]⁺ |
| 160 | 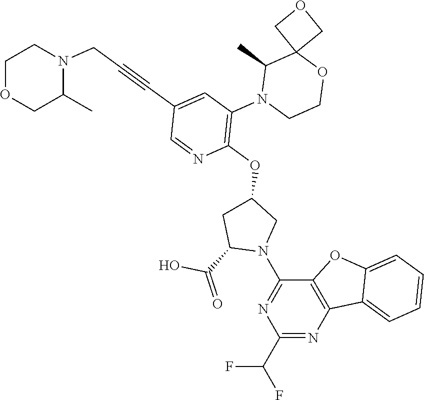 [SMILES: CC1COCCN1CC#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(3- methylmorpholino)prop-1-yn-1-yl)pyridin-2- yl)oxy)pyrrolidine-2-carboxylic acid; | 4-(but-2-yn-1-yl)-3- methylmorpholine | mass calcd for C₃₆H₃₈F₂N₆O₇ 704.28 m/z found 705.40 [M + H]⁺ |
| 161 | 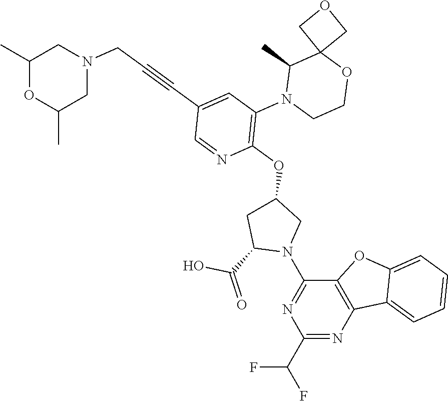 [SMILES: CC1CN(CC#Cc2cnc(O[C@H]3C[C@@H](C(=O)O)N(c4nc(C(F)F)nc5c4oc4ccccc45)C3)c(N3CCOC4(COC4)[C@@H]3C)c2)CC(C)O1] (2S,4S)-1-(2-(Difluoromethyl)benzofuro[3,2- d]pyrimidin-4-yl)-4-((5-(3-(2,6- dimethylmorpholino)prop-1-yn-1-yl)-3-((S)-9- methyl-2,5-dioxa-8-azaspiro[3.5]nonan-8- yl)pyridin-2-yl)oxy)pyrrolidine-2-carboxylic acid; | 2,6-dimethyl-4-(prop-2- yn-1-yl)morpholine | mass calcd for C₃₇H₄₀F₂N₆O₇ 718.29 m/z found 719.40 [M + H]⁺ |
| 162 | 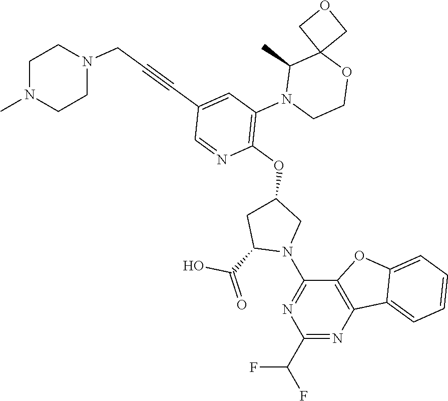 [SMILES: C[C@@H]1N(c2cc(C#CCN3CCN(C)CC3)cnc2O[C@H]2C[C@@H](C(=O)O)N(c3nc(C(F)F)nc4c3oc3ccccc34)C2)CCOC12COC2] (2S,4S)-1-(2-(Difluoromethyl)benzofuro[3,2- d]pyrimidin-4-yl)-4-((3-((S)-9-methyl-2,5-dioxa-8- azaspiro[3.5]nonan-8-yl)-5-(3-(4-methylpiperazin-1- yl)prop-1-yn-1-yl)pyridin-2-yl)oxy)pyrrolidine-2- carboxylic acid; | 1-methyl-4-(prop-2-yn- 1-yl)piperazine | mass calcd for C₃₆H₃₉F₂N₇O₆ 703.29 m/z found 704.50 [M + H]⁺ |
| 165 | 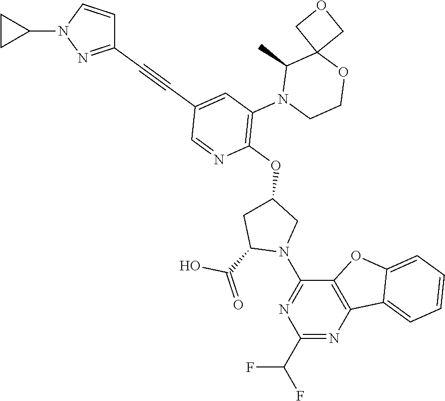 [SMILES: C[C@@H]1N(c2cc(C#Cc3ccn(C4CC4)n3)cnc2O[C@H]2C[C@@H](C(=O)O)N(c3nc(C(F)F)nc4c3oc3ccccc34)C2)CCOC12COC2] (2S,4S)-4-[[5-[2-(1-Cyclopropylpyrazol-3-yl)ethynyl]- 3-[(9S)-9-methyl-2,5-dioxa-8-azaspiro[3.5]nonan-8- yl]-2-pyridyl]oxy]-1-[2- (difluoromethyl)benzofuro[3,2-d]pyrimidin-4- yl]pyrrolidine-2-carboxylic acid; | 1-cyclopropyl-3-ethynyl- 1H-pyrazole | mass calcd for C₃₆H₃₃F₂N₇O₆ 697.25 m/z found 698.40 [M + H]⁺ |
| 166 | 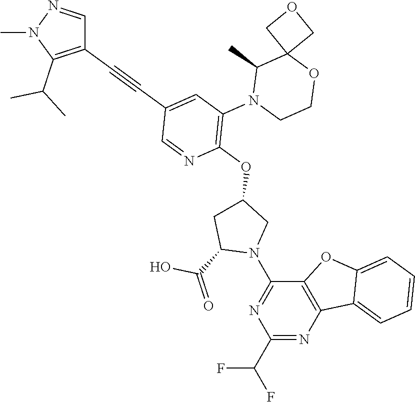 [SMILES: CC(C)c1c(C#Cc2cnc(O[C@H]3C[C@@H](C(=O)O)N(c4nc(C(F)F)nc5c4oc4ccccc45)C3)c(N3CCOC4(COC4)[C@@H]3C)c2)cnn1C] (2S,4S)-1-[2-(Difluoromethyl)benzofuro[3,2- d]pyrimidin-4-yl]-4-[[5-[2-(5-isopropyl-1-methyl- pyrazol-4-yl)ethynyl]-3-[(9S)-9-methyl-2,5-dioxa-8- azaspiro[3.5]nonan-8-yl]-2-pyridyl]oxy]pyrrolidine- 2-carboxylic acid; | 4-ethynyl-5-isopropyl-1- methyl-1H-pyrazole | mass calcd for C₃₇H₃₇F₂N₇O₆ 713.28 m/z found 714.40 [M + H]⁺ |
| 169 | 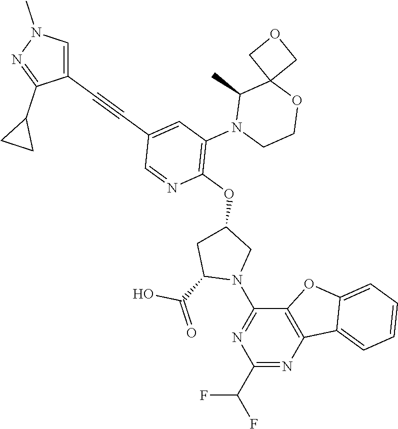 [SMILES: C[C@@H]1N(c2cc(C#Cc3cn(C)nc3C3CC3)cnc2O[C@H]2C[C@@H](C(=O)O)N(c3nc(C(F)F)nc4c3oc3ccccc34)C2)CCOC12COC2] (2S,4S)-4-[[5-[2-(3-Cyclopropyl-1-methyl-pyrazol-4- yl)ethynyl]-3-[(9S)-9-methyl-2,5-dioxa-8- azaspiro[3.5]nonan-8-yl]-2-pyridyl]oxy]-1-[2- (difluoromethyl)benzofuro[3,2-d]pyrimidin-4- yl]pyrrolidine-2-carboxylic acid; | 3-cyclopropyl-4-ethynyl- 1-methyl-1H-pyrazole | mass calcd for C₃₇H₃₅F₂N₇O₆ 711.26m/z found 712.50 [M + H]⁺ |
| 172 | 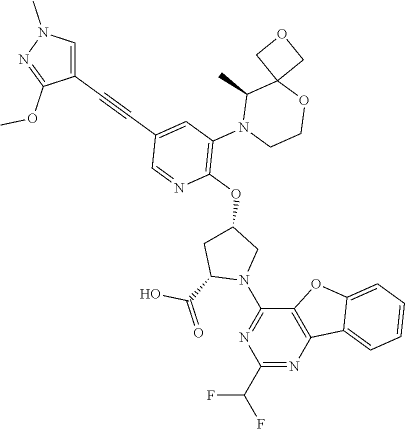 [SMILES: COc1nn(C)cc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[5-[2-(3-methoxy-1-methyl- pyrazol-4-yl)ethynyl]-3-[(9S)-9-methyl-2,5-dioxa-8- azaspiro[3.5]nonan-8-yl]-2-pyridyl]oxy]pyrrolidine- 2-carboxylic acid; | 4-ethynyl-3-methoxy-1- methyl-1H-pyrazole | mass calcd for C₃₅H₃₃F₂N₇O₇ 701.24 m/z found 702.40 [M + H]⁺ |
| 173 | 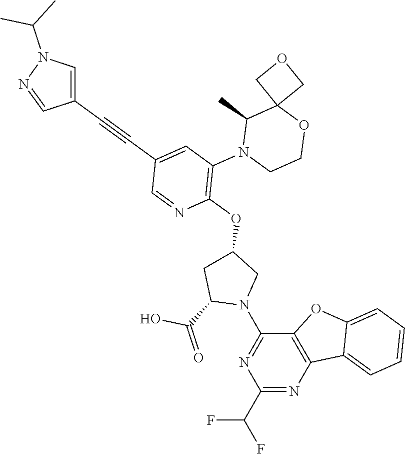 [SMILES: CC(C)n1cc(C#Cc2cnc(O[C@H]3C[C@@H](C(=O)O)N(c4nc(C(F)F)nc5c4oc4ccccc45)C3)c(N3CCOC4(COC4)[C@@H]3C)c2)cn1] (2S,4S)-1-[2-(Difluoromethyl)benzofuro[3,2- d]pyrimidin-4-yl]-4-[[5-[2-(1-isopropylpyrazol-4- yl)ethynyl]-3-[(9S)-9-methyl-2,5-dioxa-8- azaspiro[3.5]nonan-8-yl]-2-pyridyl]oxy]pyrrolidine- 2-carboxylic acid; | 4-ethynyl-1-isopropyl- 1H-pyrazole | mass calcd for C₃₆H₃₅F₂N₇O₇ 699.26 m/z found 700.40 [M + H]⁺ |
| 174 | 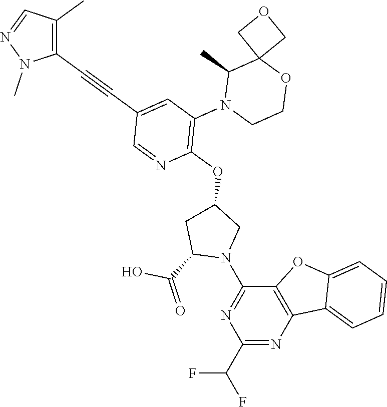 [SMILES: Cc1cnn(C)c1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[5-[2-(2,4-dimethylpyrazol-3- yl)ethynyl]-3-[(9S)-9-methyl-2,5-dioxa-8- azaspiro[3.5]nonan-8-yl]-2-pyridyl]oxy]pyrrolidine- 2-carboxylic acid; | 5-ethynyl-1,4-dimethyl- 1H-pyrazole | mass calcd for C₃₅H₃₃F₂N₇O₆ 685.25 m/z found 686.40 [M + H]⁺ |
| 175 | 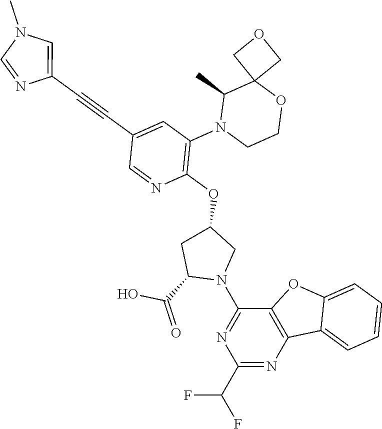 [SMILES: C[C@@H]1N(c2cc(C#Cc3cn(C)cn3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(1-methylimidazol-4- yl)ethynyl]-2-pyridyl]oxy]pyrrolidine-2-carboxylic acid; | 4-ethynyl-1-methyl-1H- imidazole | mass calcd for C₃₄H₃₁F₂N₇O₆ 671.23 m/z found 672.40 [M + H]⁺ |
| 176 | 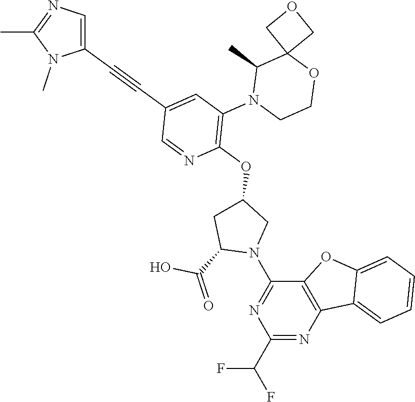 [SMILES: Cc1ncc(C#Cc2cnc(O[C@H]3C[C@@H](C(=O)O)N(c4nc(C(F)F)nc5c4oc4ccccc45)C3)c(N3CCOC4(COC4)[C@@H]3C)c2)n1C] (2S,4S)-1-[2-(Difluoromethyl)benzofuro[3,2- d]pyrimidin-4-yl]-4-[5-[2-(2,3-dimethylimidazol-4- yl)ethynyl]-3-[(9S)-9-methyl-2,5-dioxa-8- azaspiro[3.5]nonan-8-yl]-2-pyridyl]oxy]pyrrolidine- 2-carboxylic acid; | 5-ethynyl-1,2-dimethyl- 1H-imidazole | mass calcd for C₃₅H₃₃F₂N₇O₆ 685.25 m/z found 686.40 [M + H]⁺ |
| 177 | 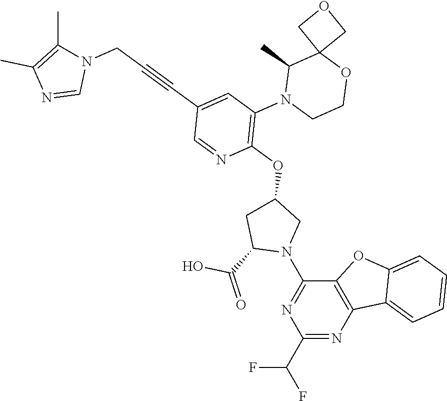 [SMILES: Cc1ncn(CC#Cc2cnc(O[C@H]3C[C@@H](C(=O)O)N(c4nc(C(F)F)nc5c4oc4ccccc45)C3)c(N3CCOC4(COC4)[C@@H]3C)c2)c1C] (2S,4S)-1-(2-(Difluoromethyl)benzofuro[3,2- d]pyrimidin-4-yl)-4-((5-(3-(4,5-dimethyl-1H- imidazol-1-yl)prop-1-yn-1-yl)-3-((S)-9-methyl-2,5- dioxa-8-azaspiro[3.5]nonan-8-yl)pyridin-2- yl)oxy)pyrrolidine-2-carboxylic acid; | 4,5-dimethyl-1-(prop-2- yn-1-yl)-1H-imidazole | mass calcd for C₃₆H₃₅F₂N₇O₆ 699.26 m/z found 700.50 [M + H]⁺ |
| 178 | 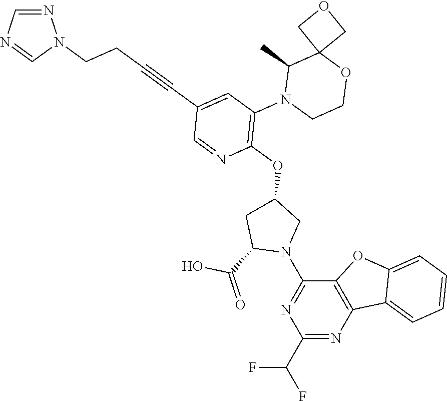 [SMILES: C[C@@H]1N(c2cc(C#CCCn3cncn3)cnc2O[C@H]2C[C@@H](C(=O)O)N(c3nc(C(F)F)nc4c3oc3ccccc34)C2)CCOC12COC2] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[4-(1,2,4-triazol-1- yl)but-1-ynyl]-2-pyridyl]oxy]pyrrolidine-2-carboxylic acid; | 1-(but-3-yn-1-yl)-1H- 1,2,4-triazole | mass calcd for C₃₄H₃₂F₂N₈O₆ 686.24 m/z found 687.40 [M + H]⁺ |
| 179 | 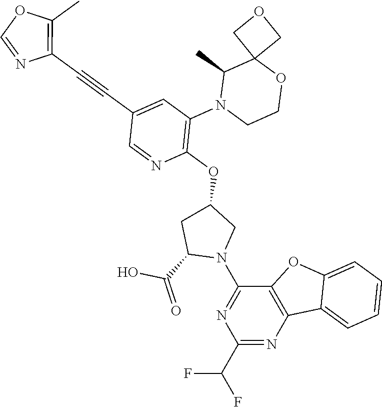 [SMILES: Cc1ocnc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(5-methyloxazol-4- yl)ethynyl]-2-pyridyl]oxy]pyrrolidine-2-carboxylic acid; | 4-ethynyl-5- methyloxazole | mass calcd for C₃₄H₃₀F₂N₆O₇ 672.21 m/z found 673.40 [M + H]⁺ |
| 180 | 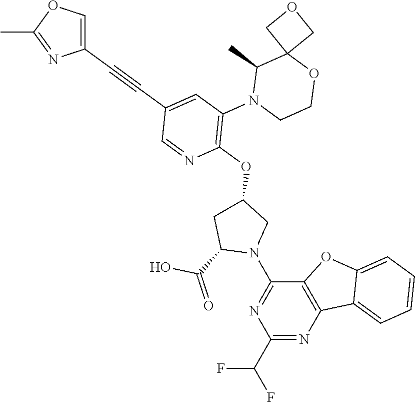 [SMILES: Cc1nc(C#Cc2cnc(O[C@H]3C[C@@H](C(=O)O)N(c4nc(C(F)F)nc5c4oc4ccccc45)C3)c(N3CCOC4(COC4)[C@@H]3C)c2)co1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(2-methyloxazol-4- yl)ethynyl]-2-pyridyl]oxy]pyrrolidine-2-carboxylic acid; | 4-ethynyl-2- methyloxazole | mass calcd for C₃₄H₃₀F₂N₆O₇ 672.21 m/z found 673.40 [M + H]⁺ |
| 181 | 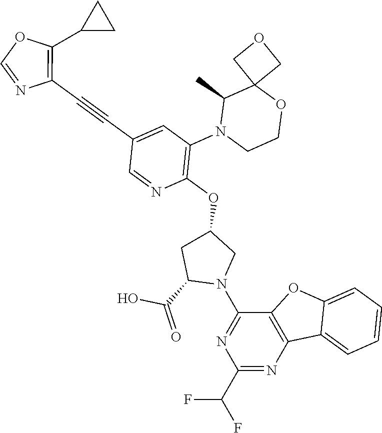 [SMILES: C[C@@H]1N(c2cc(C#Cc3ncoc3C3CC3)cnc2O[C@H]2C[C@@H](C(=O)O)N(c3nc(C(F)F)nc4c3oc3ccccc34)C2)CCOC12COC2] (2S,4S)-4-[[5-[2-(5-Cyclopropyloxazol-4-yl)ethynyl]- 3-[(9S)-9-methyl-2,5-dioxa-8-azaspiro[3.5]nonan-8- yl]-2-pyridyl]oxy]-1-[2- (difluoromethyl)benzofuro[3,2-d]pyrimidin-4- yl]pyrrolidine-2-carboxylic acid; | 5-cyclopropyl-4- ethynyloxazole | mass calcd for C₃₆H₃₂F₂N₆O₇ 698.23 m/z found 699.50 [M + H]⁺ |
| 183 | 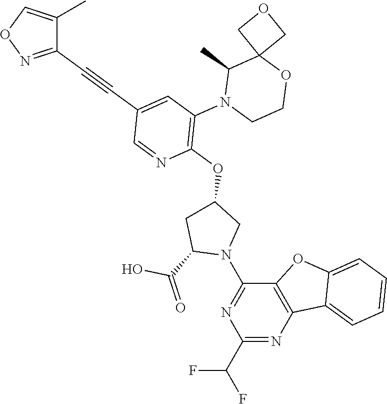 [SMILES: Cc1conc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(4-methylisoxazol-3- yl)ethynyl]-2-pyridyl]oxy]pyrrolidine-2-carboxylic acid; | 3-ethynyl-4- methylisoxazole | mass calcd for C₃₄H₃₀F₂N₆O₇ 672.21 m/z found 673.40 [M + H]⁺ |
| 184 | 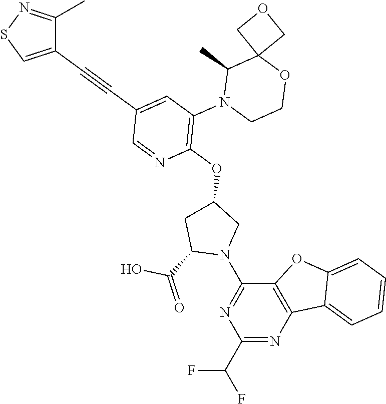 [SMILES: Cc1nscc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(3-methylisothiazol- 4-yl)ethynyl]-2-pyridyl]oxy]pyrrolidine-2-carboxylic acid; | 4-ethynyl-3- methylisothiazole | mass calcd for C₃₄H₃₀F₂N₆O₆S 688.19 m/z found 689.40 [M + H]⁺ |
| 185 | 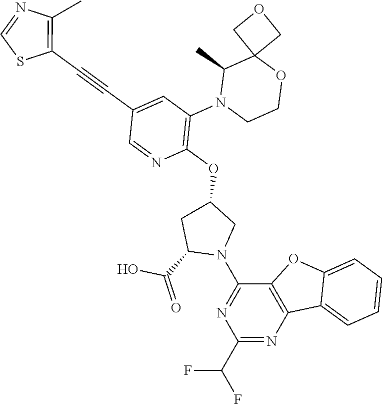 [SMILES: Cc1ncsc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(4-methylthiazol-5- yl)ethynyl]-2-pyridyl]oxy]pyrrolidine-2-carboxylic acid; | 5-ethynyl-4- methylthiazole | mass calcd for C₃₄H₃₀F₂N₆O₆S 688.19 m/z found 689.40 [M + H]⁺ |
| 186 | 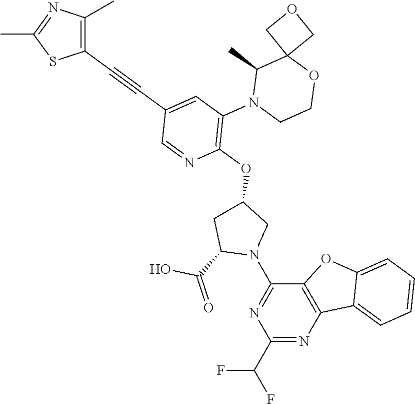 [SMILES: Cc1nc(C)c(C#Cc2cnc(O[C@H]3C[C@@H](C(=O)O)N(c4nc(C(F)F)nc5c4oc4ccccc45)C3)c(N3CCOC4(COC4)[C@@H]3C)c2)s1] (2S,4S)-1-[2-(Difluoromethyl)benzofuro[3,2- d]pyrimidin-4-yl]-4-[[5-[2-(2,4-dimethylthiazol-5- yl)ethynyl]-3-[(9S)-9-methyl-2,5-dioxa-8- azaspiro[3.5]nonan-8-yl]-2-pyridyl]oxy]pyrrolidine- 2-carboxylic acid; | 5-ethynyl-2,4- dimethylthiazole | mass calcd for C₃₅H₃₂F₂N₆O₆S 702.21 m/z found 703.30 [M + H]⁺ |
| 187 | 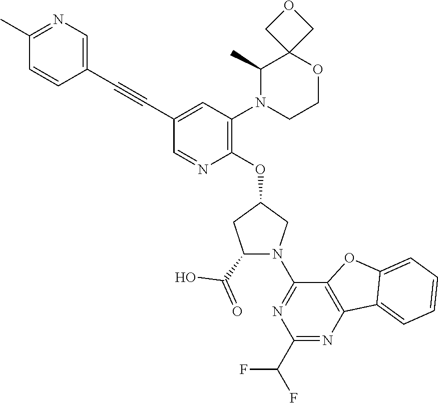 [SMILES: Cc1ccc(C#Cc2cnc(O[C@H]3C[C@@H](C(=O)O)N(c4nc(C(F)F)nc5c4oc4ccccc45)C3)c(N3CCOC4(COC4)[C@@H]3C)c2)cn1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(6-methyl-3- pyridyl)ethynyl]-2-pyridyl]oxy]pyrrolidine-2- carboxylic acid; | 5-ethynyl-2- methylpyridine | mass calcd for C₃₆H₃₂F₂N₆O₆ 682.24 m/z found 683.40 [M + H]⁺ |
| 191 | 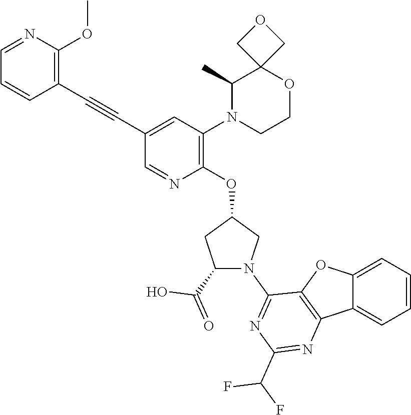 [SMILES: COc1ncccc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[5-[2-(2-methoxy-3- pyridyl)ethynyl]-3-[(9S)-9-methyl-2,5-dioxa-8- azaspiro[3.5]nonan-8-yl]-2-pyridyl]oxy]pyrrolidine- 2-carboxylic acid; | 3-ethynyl-2- methoxypyridine | mass calcd for C₃₆H₃₂F₂N₆O₇ 698.23 m/z found 699.40 [M + H]⁺ |
| 192 | 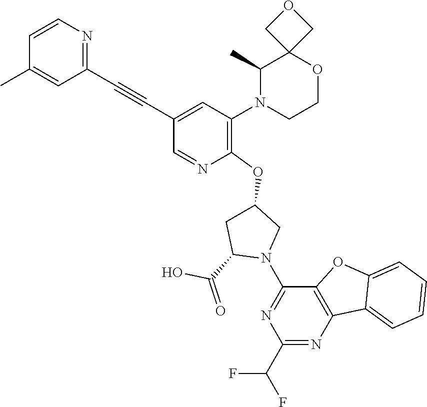 [SMILES: Cc1ccnc(C#Cc2cnc(O[C@H]3C[C@@H](C(=O)O)N(c4nc(C(F)F)nc5c4oc4ccccc45)C3)c(N3CCOC4(COC4)[C@@H]3C)c2)c1] (2S,4S)-1-[2-(Difluoromethyl)benzofuro[3,2- d]pyrimidin-4-yl]-4-[[3-[(9S)-9-methyl-2,5-dioxa-8- azaspiro[3.5]nonan-8-yl]-5-[2-(4-methyl-2- pyridyl)ethynyl]-2-pyridyl]oxy]pyrrolidine-2- carboxylic acid; | 2-ethynyl-4- methylpyridine | mass calcd for C₃₆H₃₂F₂N₆O₆ 682.24 m/z found 683.40 [M + H]⁺ |
| 193 | 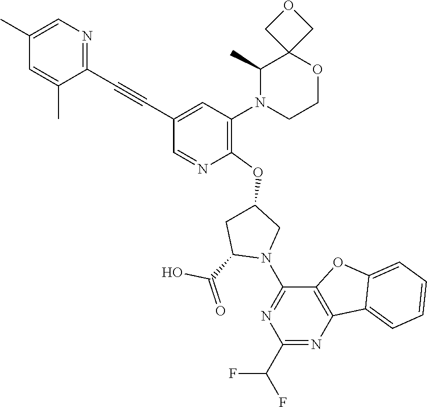 [SMILES: Cc1cnc(C#Cc2cnc(O[C@H]3C[C@@H](C(=O)O)N(c4nc(C(F)F)nc5c4oc4ccccc45)C3)c(N3CCOC4(COC4)[C@@H]3C)c2)c(C)c1] (2S,4S)-1-[2-(Difluoromethyl)benzofuro[3,2- d]pyrimidin-4-yl]-4-[[5-[2-(3,5-dimethyl-2- pyridyl)ethynyl]-3-[(9S)-9-methyl-2,5-dioxa-8- azaspiro[3.5]nonan-8-yl]-2-pyridyl]oxy]pyrrolidine- 2-carboxylic acid; | 2-ethynyl-3,5- dimethylpyridine | mass calcd for C₃₇H₃₄F₂N₆O₆ 696.25 m/z found 697.40 [M + H]⁺ |
| 194 | 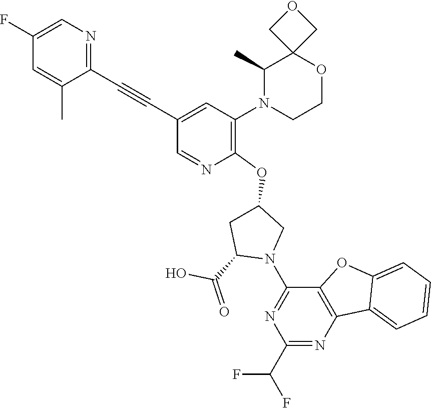 [SMILES: Cc1cc(F)cnc1C#Cc1cnc(O[C@H]2C[C@@H](C(=O)O)N(c3nc(C(F)F)nc4c3oc3ccccc34)C2)c(N2CCOC3(COC3)[C@@H]2C)c1] (2S,4S)-1-[2-(Difluoromethyl)benzofuro[3,2- d]pyrimidin-4-yl]-4-[[5-[2-(5-fluoro-3-methyl-2- pyridyl)ethynyl]-3-[(9S)-9-methyl-2,5-dioxa-8- azaspiro[3.5]nonan-8-yl]-2-pyridyl]oxy]pyrrolidine- 2-carboxylic acid. | 2-ethynyl-5-fluoro-3- methylpyridine | mass calcd for C₃₆H₃₁F₃N₆O₆ 700.23 |

Examples 118, 120, 148, 150, 167, 168, 170-171, and 195-196 in Table 17 were prepared in a manner analogous to Example 1, using the appropriate alkyne and the corresponding halide under Synogashira coupling conditions with a catalyst and ligand such as XPhos Pd G3 & XPhos, a base such as Cs₂CO₃, in CH₃CN or DMF, at temperatures ranging from rt to 100° C. for period of 1-24 hours.

TABLE 17

| Ex | | | |
|---|---|---|---|
| # | Structure | Alkyne/Halide | MS and ¹H NMR |
| 118 | 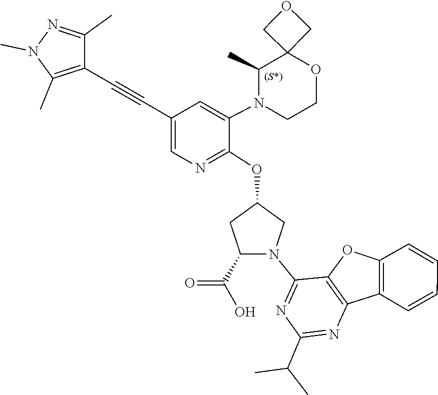 [SMILES: Cc1nn(C)c(C)c1C#Cc1cnc(O[C@H]2C[C@@H](C(=O)O)N(c3nc(C(C)C)nc4c3oc3ccccc34)C2)c(N2CCOC3(COC3)[C@@H]2C)c1] | 4-ethynyl-1,3,5- trimethyl-1H- pyrazole and (2S,4S)-4-((5- bromo-3-((S*)-9- methyl-2,5-dioxa- 8-azaspiro [3.5]nonan- 8-yl)pyridin-2- yl)oxy)-1-(2- isopropylbenzo- furo[3,2- d]pyrimidin-4- yl)pyrrolidine-2- carboxylic acid (Intermediate 41) | LCMS (ESI): mass calcd for C₃₈H₄₁N₇O₆, 691.3; m/z found, 692.4 [M + H]⁺. ¹H NMR (400 MHz, DMSO-d₆) δ 8.08 (d, J = 7.6 Hz, 1H), 7.95 (d, J = 2.0 Hz, 1H), 7.74-7.68 (m, 1H), 7.68-7.62 (m, 1H), 7.49-7.44 (m, 1H), 7.22 (d, J = 2.0 Hz, 1H), 5.71-5.61 (m, 1H), 5.15- 5.01 (m, 1H), 4.69-4.63 (m, 1H), 4.62- 4.51 (m, 2H), 4.39-4.33 (m, 1H), 4.33-4.26 (m, 1H), 4.25-4.16 (m, 1H), 4.14-4.08 (m, 1H), 3.80-3.73 (m, 1H), 3.68 (s, 3H), 3.66-3.58 (m, 1H), 3.32-3.23 (m, 1H), 3.13-3.11 (m, 1H), 3.02-2.94 (m, 1H), 2.76- 2.68 (m, 1H) 2.59-2.52 (m, 1H), 2.31 (s, 3H), 2.19 (s, 3H), 1.32 (dd, J = 2.4, 6.8 Hz, 6H), 0.84 (d, J = 6.4 Hz, 3H). |
| | (2S,4S)-1-(2-isopropylbenzofuro[3,2- | | |
| | d]pyrimidin-4-yl)-4-((3-(S*)-9-methyl- | | |
| | 2,5-dioxa-8-azaspiro[3.5]nonan-8-yl)- | | |
| | 5-((1,3,5-trimethyl-1H-pyrazol-4- | | |
| | yl)ethynyl)pyridin-2- | | |
| | yl)oxy)pyrrolidine-2-carboxylic acid | | |
| 120 | 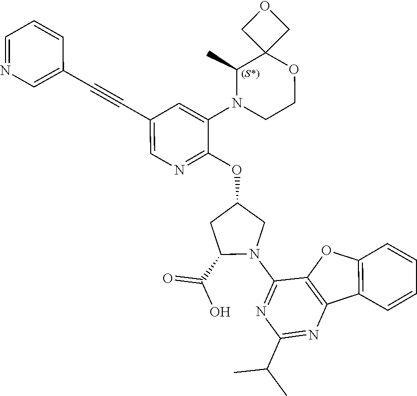 [SMILES: CC(C)c1nc(N2C[C@@H](Oc3ncc(C#Cc4cccnc4)cc3N3CCOC4(COC4)[C@@H]3C)C[C@H]2C(=O)O)c2oc3ccccc3c2n1] | 3-ethynylpyridine and (2S,4S)-4-((5- bromo-3-((S*)-9- methyl-2,5-dioxa- 8-azaspiro [3.5]nonan- 8-yl)pyridin-2- yl)oxy)-1-(2- isopropylbenzo- furo[3,2- d]pyrimidin-4- yl)pyrrolidine-2- carboxylic acid (Intermediate 41) | LCMS (ESI): mass calcd for C₃₇H₃₆N₆O₆, 660.3; m/z found, 661.4 [M + H]⁺. ¹H NMR (400 MHz, DMSO-d₆) δ 8.75 (s, 1H), 8.63-8.56 (m, 1H), 8.13-8.05 (m, 2H), 8.00-7.93 (m, 1H), 7.75- 7.62 (m, 2H), 7.52-7.41 (m, 2H), 7.34 (s, 1H), 5.77-5.62 (m, 1H), 5.19-5.02 (m, 1H), 4.72-4.63 (m, 1H), 4.60- 4.48 (m, 2H), 4.41-4.29 (m, 2H), 4.28- 4.18 (m, 1H), 4.16-4.09 (m, 1H), 3.84-3.74 (m, 1H), 3.71-3.63 (m, 1H), 3.34-3.30 (m, 1H), 3.00-2.95 (m, 2H), 2.77-2.71 (m, 1H), 2.62- 2.55 (m, 1H), 1.37-1.31 (m, 6H), 0.85 (br d, J = 6.8 Hz, 3H). |
| | (2S,4S)-1-(2-Isopropylbenzofuro[3,2- | | |
| | d]pyrimidin-4-yl)-4-((3-(S*)-9-methyl- | | |
| | 2,5-dioxa-8-azaspiro[3.5]nonan-8-yl)- | | |
| | 5-(pyridin-3-ylethynyl)pyridin-2- | | |
| | yl)oxy)pyrrolidine-2-carboxylic acid | | |
| 148 | 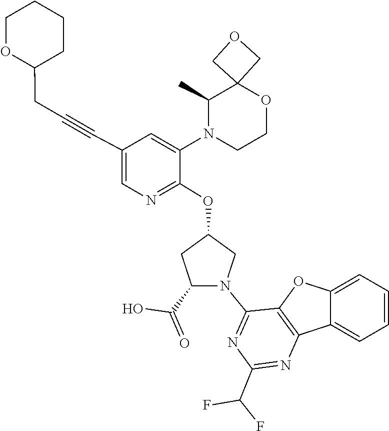 [SMILES: C[C@@H]1N(c2cc(C#CCC3CCCCO3)cnc2O[C@H]2C[C@@H](C(=O)O)N(c3nc(C(F)F)nc4c3oc3ccccc34)C2)CCOC12COC2] | 2-(prop-2-yn-1- yl)tetrahydro-2H- pyran and (2S,4S)-4-((5- Bromo-3-((S)-9- methyl-2,5-dioxa- 8-azaspiro [3.5]nonan- 8-yl)pyridin-2- yl)oxy)-1-(2- (difluoromethyl) benzofuro[3,2- d]pyrimidin-4- yl)pyrrolidine-2- carboxylic acid (Intermediate 12). | mass calcd for C₃₆H₃₇F₂N₅O₇ 689.30 m/z found, 690.40 [M + H]⁺. |
| | (2S,4S)-1-(2- | | |
| | (Difluoromethyl)benzofuro[3,2- | | |
| | d]pyrimidin-4-yl)-4-((3-((S)-9-methyl- | | |
| | 2,5-dioxa-8-azaspiro[3.5]nonan-8-yl)- | | |
| | 5-(3-(tetrahydro-2H-pyran-2-yl)prop- | | |
| | 1-yn-1-yl)pyridin-2-yl)oxy)pyrrolidine- | | |
| | 2-carboxylic acid; | | |
| 150 | 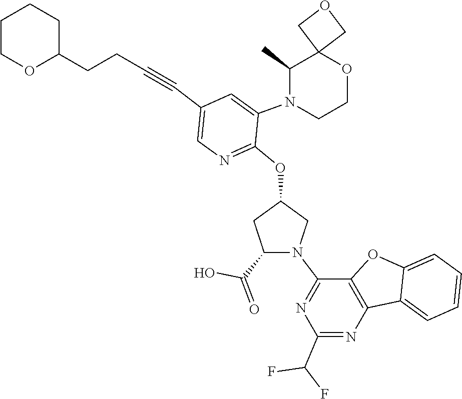 [SMILES: C[C@@H]1N(c2cc(C#CCCC3CCCCO3)cnc2O[C@H]2C[C@@H](C(=O)O)N(c3nc(C(F)F)nc4c3oc3ccccc34)C2)CCOC12COC2] | 2-(but-3-yn-1- yl)tetrahydro-2H- pyran and (2S,4S)-4-((5- Bromo-3-((S)-9- methyl-2,5-dioxa- 8-azaspiro [3.5]nonan- 8-yl)pyridin-2- yl)oxy)-1-(2- (difluoromethyl) benzofuro[3,2- d]pyrimidin-4- yl)pyrrolidine-2- carboxylic acid (Intermediate 12) | LCMS (ESI): mass calcd for C₃₇H₃₉F₂N₅O₇, 703.3; m/z found, 704.4 [M + H]⁺. ¹H NMR (400 MHz, DMSO-d₆) δ 8.14 (d, J = 7.6 Hz, 1H), 7.85 (d, J = 2.0 Hz, 1H), 7.80-7.75 (m, 1H), 7.75- 7.69 (m, 1H), 7.57-7.50 (m, 1H), 7.13 (d, J = 2.0 Hz, 1H), 6.76 (t, J = 54.8 Hz, 1H), 5.69-5.58 (m, 1H), 5.32-5.09 (m, 1H), 4.66-4.60 (m, 1H), 4.59- 4.47 (m, 2H), 4.37-4.32 (m, 1H), 4.31- 4.14 (m, 2H), 4.13-4.08 (m, 1H), 3.93-3.85 (m, 1H), 3.78-3.71 (m, 1H), 3.68-3.59 (m, 1H), 3.40-3.33 (m, 2H), 3.27-3.19 (m, 2H), 2.98- 2.93 (m, 1H), 2.72-2.67 (m, 1H), 2.66- 2.56 (m, 1H), 2.47-2.44 (m, 1H), 1.84-1.76 (m, 1H), 1.72-1.65 (m, 2H), 1.65-1.60 (m, 1H), 1.50-1.44 (m, 3H), 1.25-1.19 (m, 1H), 0.82 (d, J = 6.8 Hz, 3H). |
| | (2S,4S)-1-(2- | | |
| | (Difluoromethyl)benzofuro[3,2- | | |
| | d]pyrimidin-4-yl)-4-((3-((S)-9-methyl- | | |
| | 2,5-dioxa-8-azaspiro[3.5]nonan-8-yl)- | | |
| | 5-(4-(tetrahydro-2H-pyran-2-yl)but-1- | | |
| | yn-1-yl)pyridin-2-yl)oxy)pyrrolidine-2- | | |
| | carboxylic acid; | | |
| 167 | 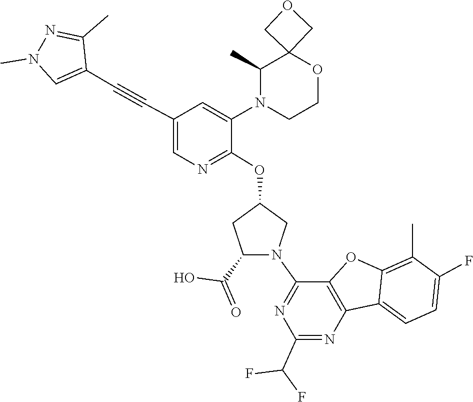 [SMILES: Cc1nn(C)cc1C#Cc1cnc(O[C@H]2C[C@@H](C(=O)O)N(c3nc(C(F)F)nc4c3oc3c(C)c(F)ccc34)C2)c(N2CCOC3(COC3)[C@@H]2C)c1] | 4-ethynyl-1,3- dimethyl-1H- pyrazole and (2S,4S)-4-((5- bromo-3-((S)-9- methyl-2,5-dioxa- 8-azaspiro [3.5]nonan- 8-yl)pyridin-2- yl)oxy)-1-(2- (difluoromethyl)- 7-fluoro-6- methylbenzofuro [3,2-d]pyrimidin- 4-yl)pyrrolidine- 2-carboxylic acid (Intermediate 36) | LCMS (ESI): mass calcd for C₃₆H₃₄F₃N₇O₆, 717.3; m/z found, 718.3 [M + H]⁺; ¹H NMR (400 MHz, DMSO-d₆) δ 8.02-7.96 (m, 1H), 7.95 (s, 1H), 7.86 (s, 1H), 7.39-7.32 (m, 1H), 7.22 (s, 1H), 6.77 (t, J = 54.8 Hz, 1H), 5.69-5.61 (m, 1H), 5.51-5.20 (m, 1H), 4.70-4.62 (m, 1H), 4.58-4.52 (m, 1H), 4.39-4.24 (m, 3H), 4.14-4.08 (m, 1H), 3.78 (s, 3H), 3.76-3.73 (m, 1H), 3.68-3.60 (m, 1H), 3.30-3.24 (m, 2H), 3.02-2.97 (m, 1H), 2.74-2.69 (m, 1H), 2.65-2.58 (m, 1H), 2.48 (s, 3H), 2.24 (s, 3H), 0.84 (d, J = 6.4 Hz, 3H); |
| | (2S,4S)-1-(2-(Difluoromethyl)-7- | | |
| | fluoro-6-methylbenzofuro[3,2- | | |
| | d]pyrimidin-4-yl)-4-((5-((1,3-dimethyl- | | |
| | 1H-pyrazol-4-yl)ethynyl)-3-((S)-9- | | |
| | methyl-2,5-dioxa-8- | | |
| | azaspiro[3.5]nonan-8-yl)pyridin-2- | | |
| | yl)oxy)pyrrolidine-2-carboxylic acid | | |
| 168 | 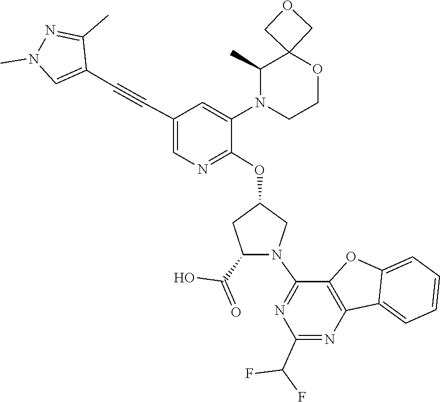 [SMILES: Cc1nn(C)cc1C#Cc1cnc(O[C@H]2C[C@@H](C(=O)O)N(c3nc(C(F)F)nc4c3oc3ccccc34)C2)c(N2CCOC3(COC3)[C@@H]2C)c1] | 4-ethynyl-1,3- dimethyl-1H- pyrazole and (2S,4S)-4-((5- bromo-3-((S)-9- methyl-2,5-dioxa- 8-azaspiro [3.5]nonan- 8-yl)pyridin-2- yl)oxy)-1-(2- (difluoromethyl) benzofuro[3,2- d]pyrimidin-4- yl)pyrrolidine-2- carboxylic acid (Intermediate 12) | LCMS (ESI): mass calcd for C₃₅H₃₃F₂N₇O₆, 685.2; m/z found, 686.3 [M + H]⁺. ¹H NMR (400 MHz, DMSO-d₆) δ = 12.42 (br s, 1H), 8.17-8.13 (m, 1H), 7.95 (d, J = 2.0 Hz, 1H), 7.86 (s, 1H), 7.82-7.76 (m, 1H), 7.76-7.70 (m, 1H), 7.57-7.50 (m, 1H), 7.22 (d, J = 2.0 Hz, 1H), 6.78 (t, J = 54.8 Hz, 1H), 5.71-5.61 (m, 1H), 5.40-5.09 (m, 1H), 4.68-4.61 (m, 1H), 4.61-4.47 (m, 2H), 4.38-4.33 (m, 1H), 4.33-4.16 (m, 2H), 4.15-4.08 (m, 1H), 3.78 (s, 3H), 3.77-3.73 (m, 1H), 3.69-3.60 (m, 1H), 3.30-3.24 (m, 1H), 3.02-2.97 (m, 1H), 2.76-2.69 (m, 1H), 2.68-2.59 (m, 1H), 2.25 (s, 3H), 0.84 (d, J = 6.4 Hz, 3H); |
| | (2S,4S)-1-(2- | | |
| | (Difluoromethyl)benzofuro[3,2- | | |
| | d]pyrimidin-4-yl)-4-((5-((1,3-dimethyl- | | |
| | 1H-pyrazol-4-yl)ethynyl)-3-((S)-9- | | |
| | methyl-2,5-dioxa-8- | | |
| | azaspiro[3.5]nonan-8-yl)pyridin-2- | | |
| | yl)oxy)pyrrolidine-2-carboxylic acid | | |
| 170 | 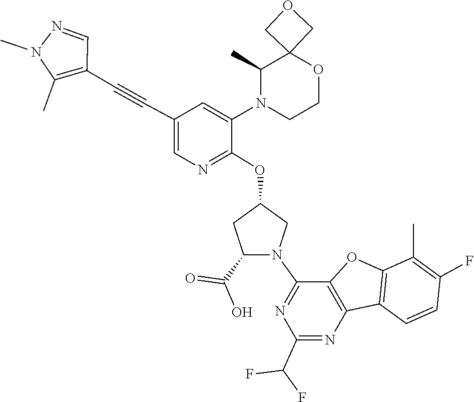 [SMILES: Cc1c(F)ccc2c1oc1c(N3C[C@@H](Oc4ncc(C#Cc5cnn(C)c5C)cc4N4CCOC5(COC5)[C@@H]4C)C[C@H]3C(=O)O)nc(C(F)F)nc12] | 4-ethynyl-1,5- dimethyl-1H- pyrazole and (2S,4S)-4-((5- bromo-3-((S)-9- methyl-2,5-dioxa- 8-azaspiro [3.5]nonan- 8-yl)pyridin-2- yl)oxy)-1-(2- (difluoromethyl)- 7-fluoro-6- methylbenzofuro [3,2-d]pyrimidin- 4-yl)pyrrolidine- 2-carboxylic acid (Intermediate 36) | LCMS (ESI): mass calcd for C₃₆H₃₄F₃N₇O₆, 717.3; m/z found, 718.3 [M + H]⁺. ¹H NMR (400 MHz, DMSO-d₆) δ 8.01-7.96 (m, 1H), 7.96-7.93 (m, 1H), 7.53 (s, 1H), 7.38-7.32 (m, 1H), 7.25-7.22 (m, 1H), 6.77 (t, J = 54.8 Hz, 1H), 5.69-5.60 (m, 1H), 5.47-5.11 (m, 1H), 4.68-4.62 (m, 1H), 4.58- 4.41 (m, 2H), 4.40-4.17 (m, 3H), 4.13- 4.09 (m, 1H), 3.78-3.74 (m, 4H), 3.68-3.61 (m, 1H), 3.29-3.25 (m, 1H), 3.05-3.04 (m, 1H), 2.75-2.66 (m, 2H), 2.48 (s, 3H), 2.36 (s, 3H), 0.84 (d, J = 6.4 Hz, 3H). |
| | (2S,4S)-1-(2-(Difluoromethyl)-7- | | |
| | fluoro-6-methylbenzofuro[3,2- | | |
| | d]pyrimidin-4-yl)-4-((5-((1,5-dimethyl- | | |
| | 1H-pyrazol-4-yl)ethynyl)-3-((S)-9- | | |
| | methyl-2,5-dioxa-8- | | |
| | azaspiro[3.5]nonan-8-yl)pyridin-2- | | |
| | yl)oxy)pyrrolidine-2-carboxylic acid | | |
| 171 | 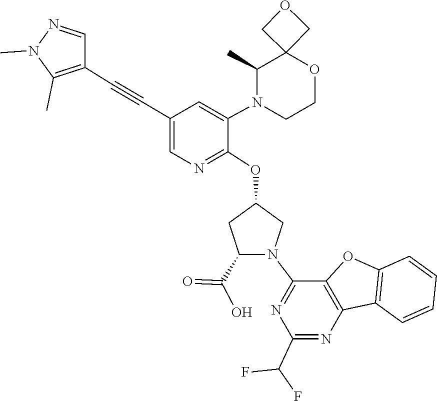 [SMILES: Cc1c(C#Cc2cnc(O[C@H]3C[C@@H](C(=O)O)N(c4nc(C(F)F)nc5c4oc4ccccc45)C3)c(N3CCOC4(COC4)[C@@H]3C)c2)cnn1C] | 4-ethynyl-1,5- dimethyl-1H- pyrazole and (2S,4S)-4-((5- bromo-3-((S)-9- methyl-2,5-dioxa- 8-azaspiro- [3.5]nonan- 8-yl)pyridin-2- yl)oxy)-1-(2- (difluoromethyl) benzofuro[3,2- d]pyrimidin-4- yl)pyrrolidine-2- carboxylic acid (Intermediate 12) | LCMS (ESI): mass calcd for C₃₅H₃₃F₂N₇O₆, 685.2; m/z found, 686.3 [M + H]⁺. ¹H NMR (400 MHz, DMSO-d₆) δ 12.48 (br s, 1H), 8.19-8.11 (m, 1H), 7.96 (d, J = 2.0 Hz, 1H), 7.84-7.76 (m, 1H), 7.76-7.68 (m, 1H), 7.61-7.50 (m, 2H), 7.24 (d, J = 2.0 Hz, 1H), 6.78 (t, J = 54.8 Hz, 1H), 5.75-5.59 (m, 1H), 5.42-5.05 (m, 1H), 4.68-4.61 (m, 1H), 4.61-4.46 (m, 2H), 4.39-4.32 (m, 1H), 4.32-4.15 (m, 2H), 4.14- 4.06 (m, 1H), 3.81-3.72 (m, 4H), 3.71- 3.58 (m, 1H), 3.33-3.23 (m, 1H), 3.03-2.97 (m, 1H), 2.76-2.69 (m, 1H), 2.68-2.58 (m, 1H), 2.37 (s, 3H), 0.84 (d, J = 6.4 Hz, 3H). |
| | (2S,4S)-1-(2- | | |
| | (Difluoromethyl)benzofuro[3,2- | | |
| | d]pyrimidin-4-yl)-4-((5-((1,5-dimethyl- | | |
| | 1H-pyrazol-4-yl)ethynyl)-3-((S)-9- | | |
| | methyl-2,5-dioxa-8- | | |
| | azaspiro[3.5]nonan-8-yl)pyridin-2- | | |
| | yl)oxy)pyrrolidine-2-carboxylic acid | | |
| 195 | 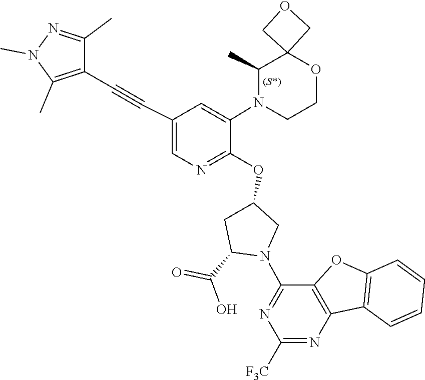 | 4-ethynyl-1,3,5- trimethyl-1H- pyrazole and (2S,4S)-4-((5- Bromo-3-((S*)-9- methyl-2,5-dioxa- 8-azaspiro [3.5]nonan- 8-yl)pyridin-2- yl)oxy)-1-(2- (trifluoromethyl) benzofuro[3,2- d]pyrimidin-4- yl)pyrrolidine-2- carboxylic acid (Intermediate 42) | LCMS (ESI): mass calcd for C₃₅H₂₉F₃N₆O₆, 686.2; m/z found, 687.3 [M + H]⁺. ¹H NMR (400 MHz, DMSO-d₆) δ 8.75 (d, J = 1.6 Hz, 1H), 8.60-8.57 (m, 1H), 8.17 (d, J = 8.0 Hz, 1H), 8.07 (d, J = 2.4 Hz, 1H), 7.97-7.93 (m, 1H), 7.86-7.78 (m, 1H), 7.78-7.72 (m, 1H), 7.59-7.52 (m, 1H), 7.48-7.43 (m, 1H), 7.34 (d, J = 2.0 Hz, 1H), 5.74- 5.63 (m, 1H), 4.71-4.59 (m, 1H), 4.58- 4.45 (m, 2H), 4.41-4.22 (m, 3H), 4.12-4.07 (m, 1H), 3.80-3.74 (m, 1H), 3.70-3.60 (m, 1H), 3.33-3.24 (m, 1H), 3.05-2.96 (m, 2H), 2.80- 2.67 (m, 2H), 0.83 (d, J = 6.8 Hz, 3H). |
| | (2S,4S)-4-((3-((S*)-9-Methyl-2,5- | | |
| | dioxa-8-azaspiro[3.5]nonan-8-yl)-5- | | |
| | ((1,3,5-trimethyl-1H-pyrazol-4- | | |
| | yl)ethynyl)pyridin-2-yl)oxy)-1-(2- | | |
| | (trifluoromethyl)benzofuro[3,2- | | |
| | d]pyrimidin-4-yl)pyrrolidine-2- | | |
| | carboxylic acid | | |
| 196 | 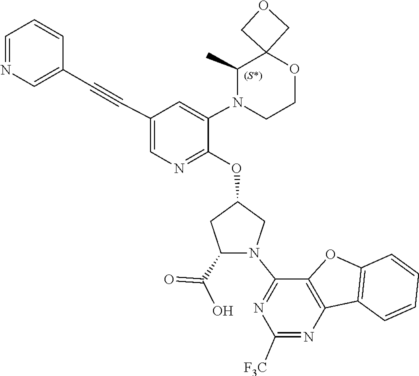 [SMILES: C[C@@H]1N(c2cc(C#Cc3cccnc3)cnc2O[C@H]2C[C@@H](C(=O)O)N(c3nc(C(F)(F)F)nc4c3oc3ccccc34)C2)CCOC12COC2] | 3-ethynylpyridine and (2S,4S)-4-((5- Bromo-3-((S*)-9- methyl-2,5-dioxa- 8-azaspiro [3.5]nonan- 8-yl)pyridin-2- yl)oxy)-1-(2- (trifluoromethyl) benzofuro[3,2- d]pyrimidin-4- yl)pyrrolidine-2- carboxylic acid (Intermediate 42) | LCMS (ESI): mass calcd for C₃₅H₂₉F₃N₆O₆, 686.2; m/z found, 687.3 [M + H]⁺. ¹H NMR (400 MHz, DMSO-d₆) δ 8.75 (d, J = 1.6 Hz, 1H), 8.60-8.57 (m, 1H), 8.17 (d, J = 8.0 Hz, 1H), 8.07 (d, J = 2.4 Hz, 1H), 7.97-7.93 (m, 1H), 7.86-7.78 (m, 1H), 7.78-7.72 (m, 1H), 7.59-7.52 (m, 1H), 7.48-7.43 (m, 1H), 7.34 (d, J = 2.0 Hz, 1H), 5.74- 5.63 (m, 1H), 4.71-4.59 (m, 1H), 4.58- 4.45 (m, 2H), 4.41-4.22 (m, 3H), 4.12-4.07 (m, 1H), 3.80-3.74 (m, 1H), 3.70-3.60 (m, 1H), 3.33-3.24 (m, 1H), 3.05-2.96 (m, 2H), 2.80- 2.67 (m, 2H), 0.83 (d, J = 6.8 Hz, 3H). |
| | (2S,4S)-4-((3-((S*)-9-Methyl-2,5- | | |
| | dioxa-8-azaspiro[3.5]nonan-8-yl)-5- | | |
| | (pyridin-3-ylethynyl)pyridin-2-yl)oxy)- | | |
| | 1-(2-(trifluoromethyl)benzofuro[3,2- | | |
| | d]pyrimidin-4-yl)pyrrolidine-2- | | |
| | carboxylic acid | | |

### Example 136: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(((R*)-tetrahydrofuran-2-yl)ethynyl)pyridin-2-yl)oxy)pyrrolidine-2-carboxylic acid

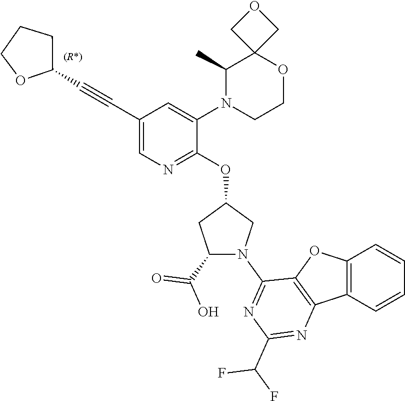 [SMILES: C[C@@H]1N(c2cc(C#C[C@H]3CCCO3)cnc2O[C@H]2C[C@@H](C(=O)O)N(c3nc(C(F)F)nc4c3oc3ccccc34)C2)CCOC12COC2]

The title compound was prepared in a manner analogous to Example 39, except 2-ethynyltetrahydrofuran was used instead of 3-(prop-2-yn-1-yl)tetrahydro-2H-pyran, the reaction time increased to overnight, and the crude product subjected to HPLC (Welch Xtimate C18, 5 μm, 150 mm×30 mm; 7 min gradient (65-95% ACN/H₂O (with 0.2% FA)) at 25 mL/min) followed by SFC (REGIS (S,S)WHELK-O1, 10 μm, 250 mm×25 mm; 5 min isocratic (50% EtOH (with 0.1% of 25% aq NH₃)/CO₂) at 80 mL/min (100 bar); column temp 35° C.) to yield two products: Example 137 (first Eluting) and the title compound (Second Eluting): (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(((R*)-tetrahydrofuran-2-yl)ethynyl)pyridin-2-yl)oxy)pyrrolidine-2-carboxylic acid as a white powder (17.4 mg, 19%). LCMS (ESI): mass calcd for C₃₄H₃₅F₂N₅O₇, 661.2; m/z found, 662.3 [M+H]⁺. ¹H NMR (400 MHz, DMSO-d₆) δ 8.14 (d, J=7.6 Hz, 1H), 7.91-7.88 (m, 1H), 7.81-7.75 (m, 1H), 7.75-7.69 (m, 1H), 7.57-7.50 (m, 1H), 7.16 (d, J=0.4 Hz, 1H), 6.77 (t, J=54.8 Hz, 1H), 5.68-5.59 (m, 1H), 5.37-5.03 (m, 1H), 4.81-4.74 (m, 1H), 4.67-4.59 (m, 1H), 4.58-4.47 (m, 2H), 4.37-4.33 (m, 1H), 4.31-4.13 (m, 2H), 4.12-4.08 (m, 1H), 3.91-3.82 (m, 1H), 3.78-3.70 (m, 2H), 3.68-3.59 (m, 1H), 3.29-3.23 (m, 1H), 2.98-2.89 (m, 1H), 2.75-2.64 (m, 2H), 2.23-2.17 (m, 1H), 2.05-1.85 (m, 3H), 0.82 (d, J=6.4 Hz, 3H).

### Example 137: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(((S*)-tetrahydrofuran-2-yl)ethynyl)pyridin-2-yl)oxy)pyrrolidine-2-carboxylic acid

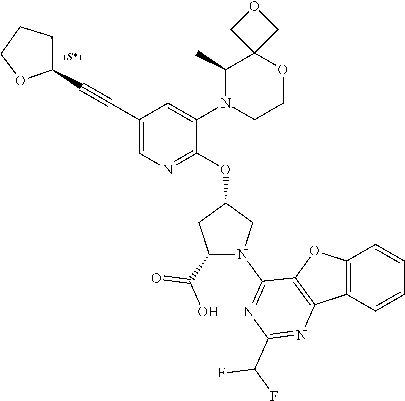 [SMILES: C[C@@H]1N(c2cc(C#C[C@@H]3CCCO3)cnc2O[C@H]2C[C@@H](C(=O)O)N(c3nc(C(F)F)nc4c3oc3ccccc34)C2)CCOC12COC2]

The title compound was isolated from Example 136, 1st Eluting: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(((S*)-tetrahydrofuran-2-yl)ethynyl)pyridin-2-yl)oxy)pyrrolidine-2-carboxylic acid as a white powder (11.9 mg, 13%). LCMS (ESI): mass calcd for C₃₄H₃₅F₂N₅O₇, 661.2; m/z found, 662.3 [M+H]⁺. ¹H NMR (400 MHz, DMSO-d₆) ¹H NMR (400 MHz, DMSO-d₆) δ 8.14 (d, J=8.0 Hz, 1H), 7.89 (d, J=1.6 Hz, 1H), 7.79-7.75 (m, 1H), 7.74-7.69 (m, 1H), 7.56-7.50 (m, 1H), 7.17 (d, J=1.6 Hz, 1H), 6.77 (t, J=54.8 Hz, 1H), 5.68-5.58 (m, 1H), 5.36-5.07 (m, 1H), 4.80-4.75 (m, 1H), 4.66-4.59 (m, 1H), 4.59-4.44 (m, 2H), 4.37-4.31 (m, 1H), 4.29-4.14 (m, 2H), 4.14-4.07 (m, 1H), 3.92-3.82 (m, 1H), 3.81-3.72 (m, 2H), 3.67-3.60 (m, 1H), 3.27-3.25 (m, 1H), 2.97-2.93 (m, 1H), 2.73-2.60 (m, 2H), 2.26-2.15 (m, 1H), 2.07-1.84 (m, 3H), 0.81 (d, J=6.8 Hz, 3H).

### Example 146: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-((R*)-tetrahydro-2H-pyran-3-yl)prop-1-yn-1-yl)pyridin-2-yl)oxy)pyrrolidine-2-carboxylic acid

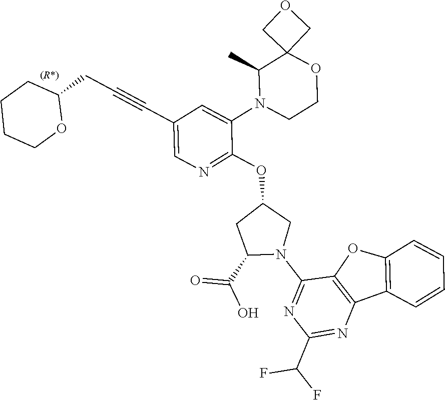 [SMILES: C[C@@H]1N(c2cc(C#CC[C@H]3CCCCO3)cnc2O[C@H]2C[C@@H](C(=O)O)N(c3nc(C(F)F)nc4c3oc3ccccc34)C2)CCOC12COC2]

The title compound was isolated from Example 147, 2nd Eluting: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-((R*)-tetrahydro-2H-pyran-3-yl)prop-1-yn-1-yl)pyridin-2-yl)oxy)pyrrolidine-2-carboxylic acid as a white solid (14.6 mg, 16%). LCMS (ESI): mass calcd for CH₃₇F₂N₅O₇, 689.3; m/z found, 690.3 [M+H]⁺. ¹H NMR (400 MHz, DMSO-d₆) δ 8.17-8.11 (m, 1H), 7.91-7.81 (m, 1H), 7.80-7.75 (m, 1H), 7.75-7.69 (m, 1H), 7.60-7.47 (m, 1H), 7.17-7.11 (m, 1H), 6.77 (t, J=54.8 Hz, 1H), 5.69-5.57 (m, 1H), 5.30-5.09 (m, 1H), 4.65-4.59 (m, 1H), 4.58-4.47 (m, 2H), 4.37-4.32 (m, 1H), 4.27-4.20 (m, 1H), 4.12-4.06 (m, 1H), 3.91-3.83 (m, 1H), 3.78-3.71 (m, 2H), 3.67-3.59 (m, 1H), 3.37-3.31 (m, 1H), 3.26-3.19 (m, 3H), 2.98-2.93 (m, 1H), 2.74-2.61 (m, 2H), 2.41-2.31 (m, 2H), 1.96-1.88 (m, 1H), 1.84-1.74 (m, 1H), 1.65-1.57 (m, 1H), 1.56-1.47 (m, 1H), 1.43-1.32 (m, 1H), 0.81 (d, J=6.8 Hz, 3H).

### Example 147: (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-((S*)-tetrahydro-2H-pyran-3-yl)prop--yn-1-yl)pyridin-2-yl)oxy)pyrrolidine-2-carboxylic acid

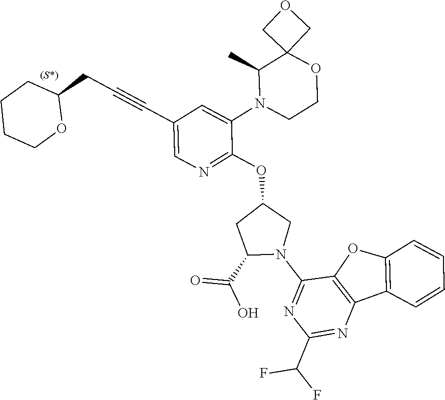 [SMILES: C[C@@H]1N(c2cc(C#CC[C@@H]3CCCCO3)cnc2O[C@H]2C[C@@H](C(=O)O)N(c3nc(C(F)F)nc4c3oc3ccccc34)C2)CCOC12COC2]

A mixture of (2S,4S)-4-((5-bromo-3-((S)-9-methyl-2,5-dioxa-8-azaspiro[3.5]nonan-8-yl)pyridin-2-yl)oxy)-1-(2-(difluoromethyl)benzofuro[3,2-d]pyrimidin-4-yl)pyrrolidine-2-carboxylic acid (Intermediate 12, 90.0 mg, 0.135 mmol), 3-(prop-2-yn-1-yl)tetrahydro-2H-pyran (35.0 mg, 0.282 mmol), Xphos (7.0 mg, 0.015 mmol), Xphos Pd G3 (12.0 mg, 0.0142 mmol), Cs₂CO₃ (135.0 mg, 0.4143 mmol), and ACN (3.0 mL) was combined and purged with N₂. The reaction mixture was stirred at 70° C. for 2 h. The resulting reaction mixture was cooled to rt, passed through a syringe filter (0.45 μm nylon membrane), and the filtrate concentrated to dryness in vacuo. The resulting residue was subjected to HPLC (Boston Green ODS, 5 μm, 150 mm×30 mm; 6 min gradient (70-100% ACN/H₂O (with 0.2% FA)) at 30 mL/min) followed by SFC (DAICEL CHIRALPAK IC, 10 μm, 250 mm×30 mm; 14 min isocratic (55% EtOH (with 0.1% of 25% aq NH₃)/CO₂) at 80 mL/min (100 bar); column temp 35° C.) to yield two products:1st Eluting (Example 147), the title compound, (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-((S*)-tetrahydro-2H-pyran-3-yl)prop-1-yn-1-yl)pyridin-2-yl)oxy)pyrrolidine-2-carboxylic acid as a white powder (Rₜ=1.40 min, 19.3 mg, 20%). LCMS (ESI): mass calcd for C₃₆H₃₇F₂N₅O₇, 689.3; m/z found, 690.3 [M+H]⁺. ¹H NMR (400 MHz, DMSO-d₆) δ 8.18-8.11 (m, 1H), 7.89-7.84 (m, 1H), 7.81-7.75 (m, 1H), 7.75-7.69 (m, 1H), 7.58-7.49 (m, 1H), 7.16-7.12 (m, 1H), 6.77 (t, J=54.8 Hz, 1H), 5.71-5.56 (m, 1H), 5.36-5.10 (m, 1H), 4.65-4.60 (m, 1H), 4.59-4.46 (m, 2H), 4.37-4.32 (m, 1H), 4.27-4.21 (m, 1H), 4.12-4.07 (m, 1H), 3.92-3.82 (m, 1H), 3.79-3.71 (m, 2H), 3.68-3.56 (m, 1H), 3.38-3.30 (m, 1H), 3.26-3.17 (m, 3H), 2.99-2.93 (m, 1H), 2.73-2.59 (m, 2H), 2.39-2.34 (m, 2H), 1.98-1.88 (m, 1H), 1.85-1.73 (m, 1H), 1.67-1.57 (m, 1H), 1.56-1.45 (m, 1H), 1.43-1.32 (m, 1H), 0.81 (d, J=6.8 Hz, 3H); and (2S,4S)-1-(2-(Difluoromethyl)benzofuro[3,2-d]pyrimidin-4-yl)-4-((3-((S)-9-methyl-2,5-dioxa-8-azaspiro[3.5]nonan-8-yl)-5-(3-((R*)-tetrahydro-2H-pyran-3-yl)prop-1-yn-1-yl)pyridin-2-yl)oxy)pyrrolidine-2-carboxylic acid (Example 146).

### Example 163: (2S,4S)-1-(2-(Difluoromethyl)-7-fluoro-6-methylbenzofuro[3,2-d]pyrimidin-4-yl)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid

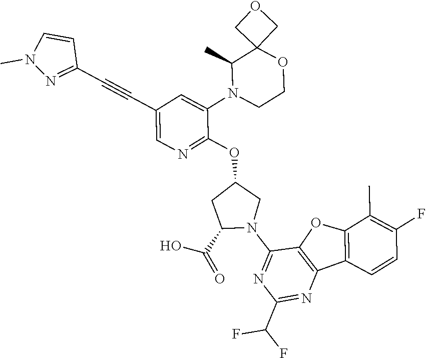 [SMILES: Cc1c(F)ccc2c1oc1c(N3C[C@@H](Oc4ncc(C#Cc5ccn(C)n5)cc4N4CCOC5(COC5)[C@@H]4C)C[C@H]3C(=O)O)nc(C(F)F)nc12]

The title compound was prepared in a manner analogous to Example 37 using (2S,4S)-4-((5-((1-methyl-1H-pyrazol-3-yl)ethynyl)-3-((S)-9-methyl-2,5-dioxa-8-azaspiro[3.5]nonan-8-yl)pyridin-2-yl)oxy)pyrrolidine-2-carboxylic acid (Intermediate 15) and 4-chloro-2-(difluoromethyl)-7-fluoro-6-methylbenzofuro[3,2-d]pyrimidine (Intermediate 31). LCMS (ESI): mass calcd for C₃₅H₃₂F₃N₇O₆, 703.4; m/z found, 704.3 [M+H]⁺. ¹H NMR (400 MHz, DMSO-d₆) δ=8.02-7.96 (m, 2H), 7.72 (d, J=2.0 Hz, 1H), 7.39-7.32 (m, 1H), 7.27 (d, J=1.6 Hz, 1H), 6.96-6.59 (m, 1H), 6.48 (d, J=2.4 Hz, 1H), 5.74-5.57 (m, 1H), 5.52-5.05 (m, 1H), 4.68-4.59 (m, 1H), 4.58-4.39 (m, 2H), 4.38-4.17 (m, 3H), 4.14-4.07 (m, 1H), 3.87 (s, 3H), 3.80-3.73 (m, 1H), 3.69-3.61 (m, 1H), 3.32-3.27 (m, 3H), 2.72 (br d, J=12.0 Hz, 1H), 2.48 (s, 3H), 0.84 (d, J=6.4 Hz, 3H). Stereochemistry was confirmed via X-Ray crystallography.

### Example 164: (2S,4S)-1-(2,6-Bis(difluoromethyl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

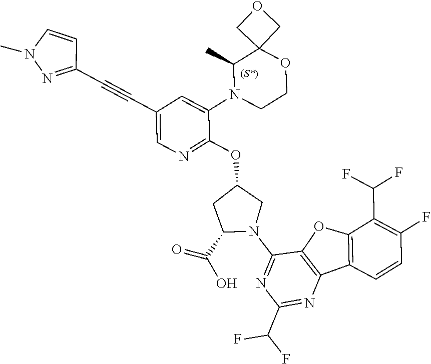 [SMILES: C[C@@H]1N(c2cc(C#Cc3ccn(C)n3)cnc2O[C@H]2C[C@@H](C(=O)O)N(c3nc(C(F)F)nc4c3oc3c(C(F)F)c(F)ccc34)C2)CCOC12COC2]

The title compound was prepared in a manner analogous to Example 37, using (2S,4S)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid trifluoroacetate (Intermediate 15) and 4-chloro-2,6-bis(difluoromethyl)benzofuro[3,2-d]pyrimidine (Intermediate 46). LCMS (ESI): mass calcd for C₃₅H₃₁F₄N₇O₆, 721.2; m/z found, 722.3 [M+H]⁺. ¹H NMR (400 MHz, DMSO-d₆) δ 8.34 (d, J=8.0 Hz, 1H), 8.00 (d, J=1.2 Hz, 1H), 7.94 (d, J=7.6 Hz, 1H), 7.73 (d, J=2.0 Hz, 1H), 7.70-7.64 (m, 1H), 7.61-7.29 (m, 1H), 7.28 (s, 1H), 6.80 (t, J=54.8 Hz, 1H), 6.49 (d, J=2.0 Hz, 1H), 5.72-5.62 (m, 1H), 4.74-4.62 (m, 1H), 4.62-4.45 (m, 2H), 4.45-4.27 (m, 2H), 4.09 (d, J=6.0 Hz, 1H), 3.88 (s, 3H), 3.81-3.73 (m, 1H), 3.69-3.58 (m, 1H), 3.34-3.15 (m, 4H), 2.78-2.65 (m, 2H), 0.84 (d, J=6.4 Hz, 3H).

Examples 182, 188, 189, 190 in Table 18 were prepared in a manner analogous to Example 110, using (2S,4S)-1-(2-(difluoromethyl)benzofuro[3,2-d]pyrimidin-4-yl)-4-((5-ethynyl-3-((S)-9-methyl-2,5-dioxa-8-azaspiro[3.5]nonan-8-yl)pyridin-2-yl)oxy)pyrrolidine-2-carboxylic acid (Intermediate 35) and the corresponding halide.

TABLE 18

| Ex # | Structure | Halide | MS and ¹H-NMR |
|---|---|---|---|
| 182 | 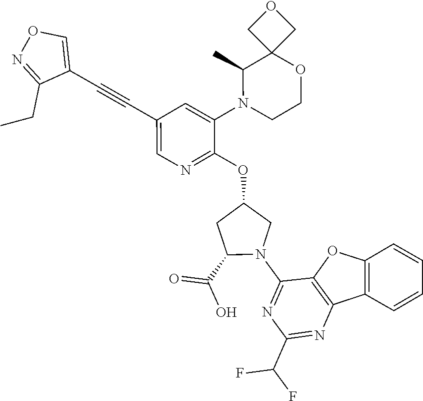 [SMILES: CCc1nocc1C#Cc1cnc(O[C@H]2C[C@@H](C(=O)O)N(c3nc(C(F)F)nc4c3oc3ccccc34)C2)c(N2CCOC3(COC3)[C@@H]2C)c1] | 4-bromo-3- ethylisoxazole | LCMS (ESI): mass calcd for C₃₅H₃₂F₂N₆O₇, 686.2; m/z found, 687.3 [M + H]⁺. ¹H NMR (400 MHz, DMSO-d₆) δ 9.16 (s, 1H), 8.14 (d, J = 7.6 Hz, 1H), 8.02 (d, J = 1.6 Hz, 1H), 7.81-7.65 (m, 2H), 7.56- 7.48 (m, 1H), 7.27 (d, J = 1.2 Hz, 1H), 6.77 (t, J = 54.8 Hz, 1H), 5.68-5.58 (m, 1H), 5.31-5.05 (m, 1H), 4.68-4.62 (m, 1H), 4.59- 4.45 (m, 2H), 4.44-4.21 (m, 3H), 4.13-4.06 (m, 1H), 3.81-3.72 (m, 1H), 3.70-3.60 (m, 1H), 3.29- 3.24 (m, 1H), 2.98-2.94 (m, 1H), 2.84-2.77 (m, 2H), 2.77- 2.71 (m, 1H), 2.69-2.58 (m, 1H), 1.32 (t, J = 7.6 Hz, 3H), 0.83 (d, J = 6.8 Hz, 3H); |
| | (2S,4S)-1-(2- | | |
| | (Difluoromethyl)benzofuro[3,2- | | |
| | d]pyrimidin-4-yl)-4-((5-((3-ethylisoxazol- | | |
| | 4-yl)ethynyl)-3-((S)-9-methyl-2,5-dioxa- | | |
| | 8-azaspiro[3.5]nonan-8-yl)pyridin-2- | | |
| | yl)oxy)pyrrolidine-2-carboxylic acid | | |
| 188 | 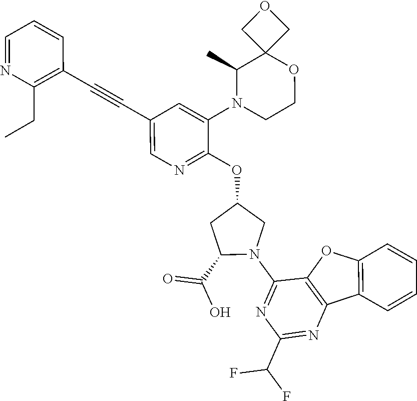 [SMILES: CCc1ncccc1C#Cc1cnc(O[C@H]2C[C@@H](C(=O)O)N(c3nc(C(F)F)nc4c3oc3ccccc34)C2)c(N2CCOC3(COC3)[C@@H]2C)c1] | 3-bromo-2- ethylpyridine | LCMS (ESI): mass calcd for C₃₇H₃₄F₂N₆O₆, 696.3; m/z found, 697.3 [M + H]⁺. ¹H NMR (400 MHz, DMSO-d₆) δ 8.54-8.46 (m, 1H), 8.15 (d, J = 8.0 Hz, 1H), 8.06 (d, J = 2.0 Hz, 1H), 7.91-7.83 (m, 1H), 7.82-7.67 (m, 2H), 7.57-7.50 (m, 1H), 7.31 (d, J = 2.0 Hz, 1H), 7.29-7.24 (m, 1H), 6.78 (t, J = 54.8 Hz, 1H), 5.72-5.63 (m, 1H), 5.40-5.10 (m, 1H), 4.70-4.63 (m, 1H), 4.63-4.45 (m, 2H), 4.40-4.34 (m, 1H), 4.32-4.25 (m, 1H), 4.13-4.08 (m, 1H), 3.82- 3.74 (m, 1H), 3.71-3.61 (m, 1H), 3.34-3.23 (m, 1H), 3.13-2.94 (m, 4H), 2.80-2.71 (m, 1H), 2.69-2.58 (m, 1H), 1.31 (t, J = 7.6 Hz, 3H), 0.84 (d, J = 6.8 Hz, 3H); |
| | (2S,4S)-1-(2- | | |
| | (Difluoromethyl)benzofuro[3,2- | | |
| | d]pyrimidin-4-yl)-4-((5-((2-ethylpyridin- | | |
| | 3-yl)ethynyl)-3-((S)-9-methyl-2,5-dioxa- | | |
| | 8-azaspiro[3.5]nonan-8-yl)pyridin-2- | | |
| | yl)oxy)pyrrolidine-2-carboxylic acid | | |
| 189 | 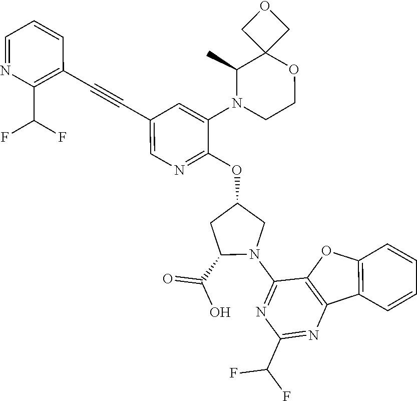 [SMILES: C[C@@H]1N(c2cc(C#Cc3cccnc3C(F)F)cnc2O[C@H]2C[C@@H](C(=O)O)N(c3nc(C(F)F)nc4c3oc3ccccc34)C2)CCOC12COC2] | 3-bromo-2- (difluoromethyl)pyridine | LCMS (ESI): mass calcd for C₃₆H₃₀F₄N₆O₆, 718.2; m/z found, 719.3 [M + H]⁺. ¹H NMR (400 MHz, DMSO-d₆) δ 12.63 (br s, 1H), 8.72-8.66 (m, 1H), 8.19-8.07 (m, 3H), 7.83-7.76 (m, 1H), 7.76- 7.70 (m, 1H), 7.67-7.61 (m, 1H), 7.58-7.50 (m, 1H), 7.38-7.09 (m, 2H), 6.78 (t, J = 54.8 Hz, 1H), 5.75-5.64 (m, 1H), 5.40-5.12 (m, 1H), 4.68-4.63 (m, 1H), 4.63- 4.51 (m, 2H), 4.41-4.20 (m, 3H), 4.15-4.06 (m, 1H), 3.83- 3.74 (m, 1H), 3.72-3.59 (m, 1H), 3.34-3.23 (m, 1H), 3.03-2.97 (m, 1H), 2.82-2.72 (m, 1H), 2.71-2.58 (m, 1H), 0.86 (d, J = 6.4 Hz, 3H); |
| | (2S,4S)-1-(2- | | |
| | (Difluoromethyl)benzofuro[3,2- | | |
| | d]pyrimidin-4-yl)-4-((5-((2- | | |
| | (difluoromethyl)pyridin-3-yl)ethynyl)-3- | | |
| | ((S)-9-methyl-2,5-dioxa-8- | | |
| | azaspiro[3.5]nonan-8-yl)pyridin-2- | | |
| | yl)oxy)pyrrolidine-2-carboxylic acid | | |
| 190 | 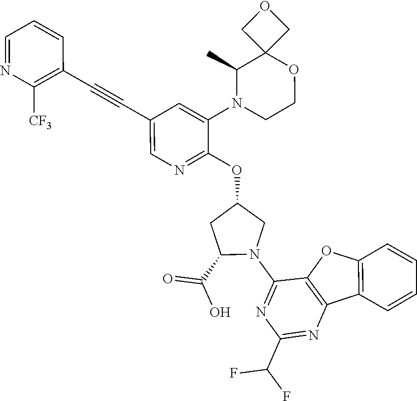 [SMILES: C[C@@H]1N(c2cc(C#Cc3cccnc3C(F)(F)F)cnc2O[C@H]2C[C@@H](C(=O)O)N(c3nc(C(F)F)nc4c3oc3ccccc34)C2)CCOC12COC2] | 3-bromo-2- (trifluoromethyl) pyridine | LCMS (ESI): mass calcd for C₃₆H₂₉F₅N₆O₆, 736.2; m/z found, 737.3 [M + H]⁺. ¹H NMR (400 MHz, DMSO-d₆) δ 8.78-8.67 (m, 1H), 8.25 (d, J = 8.0 Hz, 1H), 8.15 (d, J = 7.6 Hz, 1H), 8.05 (d, J = 2.0 Hz, 1H), 7.82-7.69 (m, 3H), 7.58- 7.49 (m, 1H), 7.29 (d, J = 2.0 Hz, 1H), 6.77 (t, J = 54.8 Hz, 1H), 5.74-5.65 (m, 1H), 5.44-5.05 (m, 1H), 4.67-4.61 (m, 1H), 4.59- 4.48 (m, 2H), 4.40-4.34 (m, 1H), 4.33-4.18 (m, 2H), 4.15-4.07 (m, 1H), 3.82-3.73 (m, 1H), 3.70- 3.62 (m, 1H), 3.31-3.26 (m, 2H), 2.79-2.73 (m, 1H), 2.70- 2.59 (m, 1H), 0.85 (d, J = 6.8 Hz, 3H); |
| | (2S,4S)-1-(2- | | |
| | (Difluoromethyl)benzofuro[3,2- | | |
| | d]pyrimidin-4-yl)-4-((3-((S)-9-methyl- | | |
| | 2,5-dioxa-8-azaspiro[3.5]nonan-8-yl)-5- | | |
| | ((2-(trifluoromethyl)pyridin-3- | | |
| | yl)ethynyl)pyridin-2-yl)oxy)pyrrolidine- | | |
| | 2-carboxylic acid | | |

### Example 197: (2S,4S)-4-((5-((1-Methyl-1H-pyrazol-3-yl)ethynyl)-3-(S*)-9-methyl-2,5-dioxa-8-azaspiro[3.5]nonan-8-yl)pyridin-2-yl)oxy)-1-(2-(1,1,1-trifluoropropan-2-yl)benzofuro[3,2-d]pyrimidin-4-yl)pyrrolidine-2-carboxylic acid

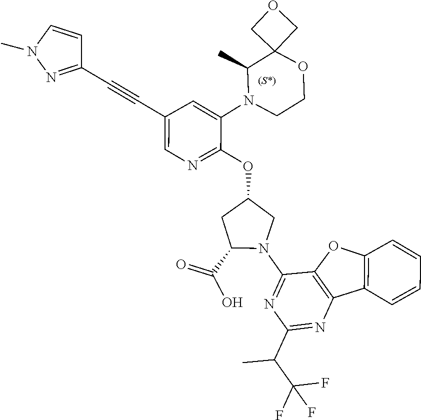 [SMILES: CC(c1nc(N2C[C@@H](Oc3ncc(C#Cc4ccn(C)n4)cc3N3CCOC4(COC4)[C@@H]3C)C[C@H]2C(=O)O)c2oc3ccccc3c2n1)C(F)(F)F]

The title compound was prepared in a manner analogous to Example 37, using 4-chloro-2-(1,1,1-trifluoropropan-2-yl)benzofuro[3,2-d]pyrimidine (Intermediate 47) instead of 4-chloro-2-(difluoromethyl)-7-fluorobenzofuro[3,2-d]pyrimidine (Intermediate 2). LCMS (ESI): mass calcd for C₃₆H₃₄F₃N₇O₆, 717.3; m/z found, 718.2 [M+H]⁺. ¹H NMR (400 MHz, DMSO-d₆) δ 8.11 (d, J=8.0 Hz, 1H), 8.00 (s, 1H), 7.78-7.72 (m, 2H), 7.72-7.66 (m, 1H), 7.55-7.46 (m, 1H), 7.28 (s, 1H), 6.49 (d, J=2.4 Hz, 1H), 5.73-5.62 (m, 1H), 5.29-4.98 (m, 1H), 4.70-4.62 (m, 1H), 4.57-4.51 (m, 1H), 4.40-4.34 (m, 1H), 4.33-4.16 (m, 2H), 4.15-4.05 (m, 1H), 3.88 (s, 3H), 3.87-3.82 (m, 1H), 3.80-3.74 (m, 1H), 3.72-3.61 (m, 1H), 3.34-3.23 (m, 2H), 3.01-2.96 (m, 1H), 2.79-2.68 (m, 1H), 2.65-2.55 (m, 1H), 1.59-1.51 (m, 3H), 0.84 (d, J=6.4 Hz, 3H).

Examples 198-205 and 207 in Table 19 were prepared in a manner analogous to Example 37, using 4-chloro-2-cyclopropylbenzofuro[3,2-d]pyrimidine Intermediate 6) and the corresponding acid.

TABLE 19

| Ex # | Structure | Acid | MS and ¹H-NMR |
|---|---|---|---|
| 198 | 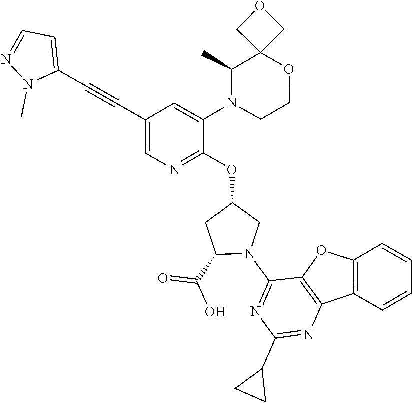 [SMILES: C[C@@H]1N(c2cc(C#Cc3ccnn3C)cnc2O[C@H]2C[C@@H](C(=O)O)N(c3nc(C4CC4)nc4c3oc3ccccc34)C2)CCOC12COC2] | (2S,4S)-4-((5-((1- methyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (Intermediate 25) | LCMS (ESI): mass calcd for C₃₆H₃₅N₇O₆, 661.3; m/z found, 662.3 [M + H]⁺. ¹H NMR (400 MHz, DMSO-d₆) δ 8.09-8.03 (m, 2H), 7.70-7.62 (m, 2H), 7.50-7.43 (m, 2H), 7.33 (d, J = 1.2 Hz, 1H), 6.57 (d, J = 1.6 Hz, 1H), 5.71- 5.64 (m, 1H), 5.10-4.94 (m, 1H), 4.67-4.63 (m, 1H), 4.56-4.47 (m, 2H), 4.36-4.29 (m, 2H), 4.26- 4.17 (m, 1H), 4.13-4.10 (m, 1H), 3.94 (s, 3H), 3.79-3.75 (m, 1H), 3.70-3.60 (m, 2H), 3.00- 2.93 (m, 2H), 2.75-2.70 (m, 1H), 2.18-2.12 (m, 1H), 1.08-1.01 (m, 2H), 0.95-0.89 (m, 2H), 0.84 (d, J = 6.4 Hz, 3H). |
| | (2S,4S)-1-(2-Cyclopropylbenzofuro[3,2- | | |
| | d]pyrimidin-4-yl)-4-((5-((1-methyl-1H- | | |
| | pyrazol-5-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 199 | 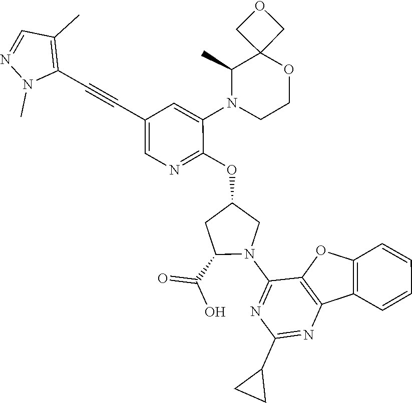 [SMILES: Cc1cnn(C)c1C#Cc1cnc(O[C@H]2C[C@@H](C(=O)O)N(c3nc(C4CC4)nc4c3oc3ccccc34)C2)c(N2CCOC3(COC3)[C@@H]2C)c1] | (2S,4S)-4-((5-((1,4- dimethyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 22) | LCMS (ESI): mass calcd for C₃₇H₃₇N₇O₆, 675.3; m/z found, 676.3 [M + H]⁺. ¹H NMR (400 MHz, DMSO-d₆) δ 8.09-8.05 (m, 2H), 7.73-7.68 (m, 1H), 7.68-7.62 (m, 1H), 7.49-7.43 (m, 1H), 7.35- 7.32 (m, 2H), 5.74-5.63 (m, 1H), 5.11-4.94 (m, 1H), 4.68- 4.64 (m, 1H), 4.58-4.47 (m, 2H), 4.39-4.34 (m, 1H), 4.33-4.27 (m, 1H), 4.26-4.17 (m, 1H), 4.15- 4.09 (m, 1H), 3.89 (s, 3H), 3.81- 3.75 (m, 1H), 3.69-3.62 (m, 1H), 3.34-3.30 (m, 1H), 2.98- 2.94 (m, 1H), 2.78-2.71 (m, 1H), 2.53-2.51 (m, 1H), 2.19-2.13 (m, 1H), 2.12 (s, 3H), 1.11-1.00 (m, 2H), 0.96-0.91 (m, 2H), 0.85 (d, J = 6.8 Hz, 3H). |
| | (2S,4S)-1-(2-Cyclopropylbenzofuro[3,2- | | |
| | d]pyrimidin-4-yl)-4-((5-((1,4-dimethyl- | | |
| | 1H-pyrazol-5-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 200 | 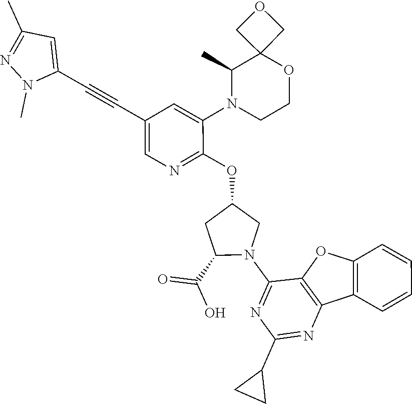 [SMILES: Cc1cc(C#Cc2cnc(O[C@H]3C[C@@H](C(=O)O)N(c4nc(C5CC5)nc5c4oc4ccccc45)C3)c(N3CCOC4(COC4)[C@@H]3C)c2)n(C)n1] | (2S,4S)-4-((5-((1,3- dimethyl-1H-pyrazol-5- yl)ethynyl)-3-((S)-9- methyl-2,5-dioxa-8- azaspiro[3.5]nonan-8- yl)pyridin-2- yl)oxy)pyrrolidine-2- carboxylic acid (intermediate 20) | LCMS (ESI): mass calcd for C₃₇H₃₇N₇O₆, 675.3; m/z found, 676.3 [M + H]⁺. ¹H NMR (400 MHz, DMSO-d₆) δ 8.09-8.04 (m, 2H), 7.72-7.60 (m, 2H), 7.49-7.43 (m, 1H), 7.33 (d, J = 1.6 Hz, 1H), 6.35 (s, 1H), 5.73-5.60 (m, 1H), 5.15-4.90 (m, 1H), 4.68-4.64 (m, 1H), 4.57-4.53 (m, 1H), 4.53- 4.44 (m, 1H), 4.37-4.34 (m, 1H), 4.33-4.28 (m, 1H), 4.26- 4.16 (m, 1H), 4.14-4.10 (m, 1H), 3.86 (s, 3H), 3.80-3.75 (m, 1H), 3.68-3.63 (m, 1H), 3.33-3.31 (m, 1H), 2.98-2.93 (m, 1H), 2.76- 2.71 (m, 1H), 2.62-2.54 (m, 1H), 2.18 (s, 3H), 2.17-2.11 (m, 1H), 1.08-1.01 (m, 2H), 0.95- 0.91 (m, 2H), 0.84 (d, J = 6.4 Hz, 3H). |
| | (2S,4S)-1-(2-Cyclopropylbenzofuro[3,2- | | |
| | d]pyrimidin-4-yl)-4-((5-((1,3-dimethyl- | | |
| | 1H-pyrazol-5-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 201 | 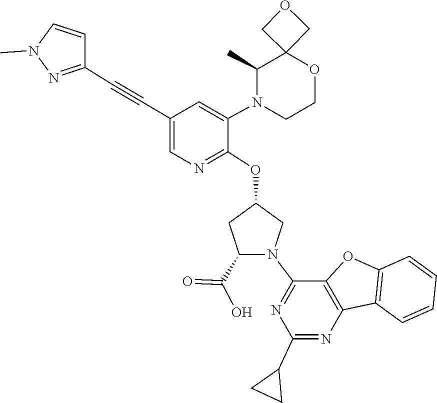 [SMILES: C[C@@H]1N(c2cc(C#Cc3ccn(C)n3)cnc2O[C@H]2C[C@@H](C(=O)O)N(c3nc(C4CC4)nc4c3oc3ccccc34)C2)CCOC12COC2] | (2S,4S)-4-((5-((1- methyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 23) | LCMS (ESI): mass calcd for C₃₆H₃₅N₇O₆, 661.3; m/z found, 662.3 [M + H]⁺. ¹H NMR (400 MHz, DMSO-d₆) δ 8.07 (d, J = 7.6 Hz, 1H), 8.00 (s, 1H), 7.75-7.69 (m, 2H), 7.68-7.62 (m, 1H), 7.50- 7.44 (m, 1H), 7.28 (s, 1H), 6.50- 6.47 (m, 1H), 5.71-5.64 (m, 1H), 5.13-4.95 (m, 1H), 4.69-4.64 (m, 1H), 4.59-4.49 (m, 2H), 4.39- 4.34 (m, 1H), 4.32-4.27 (m, 1H), 4.24-4.15 (m, 1H), 4.15- 4.10 (m, 1H), 3.88 (s, 3H), 3.83- 3.74 (m, 1H), 3.70-3.60 (m, 1H), 3.35-3.29 (m, 1H), 2.99-2.94 (m, 1H), 2.79-2.70 (m, 1H), 2.57- 2.53 (m, 1H), 2.21-2.12 (m, 1H), 1.12-1.00 (m, 2H), 0.98- 0.89 (m, 2H), 0.85 (d, J = 6.8 Hz, 3H). |
| | (2S,4S)-1-(2-Cyclopropylbenzofuro[3,2- | | |
| | d]pyrimidin-4-yl)-4-((5-((1-methyl-1H- | | |
| | pyrazol-3-yl)ethynyl)-3-((S)-9-methyl- | | |
| | 2,5-dioxa-8-azaspiro[3.5]nonan-8- | | |
| | yl)pyridin-2-yl)oxy)pyrrolidine-2- | | |
| | carboxylic acid | | |
| 202 | 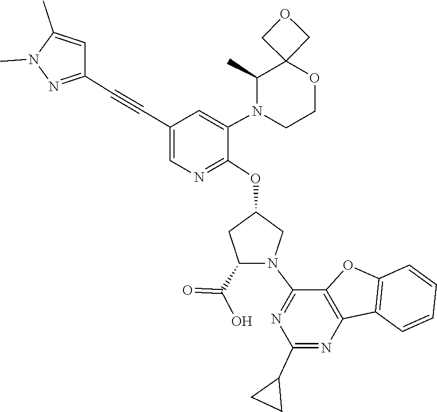 [SMILES: Cc1cc(C#Cc2cnc(O[C@H]3C[C@@H](C(=O)O)N(c4nc(C5CC5)nc5c4oc4ccccc45)C3)c(N3CCOC4(COC4)[C@@H]3C)c2)nn1C] | (2S,4S)-4-((5-((1,5- dimethyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 24) | LCMS (ESI): mass calcd for C₃₇H₃₇N₇O₆, 675.3; m/z found, 676.3 [M + H]⁺. ¹H NMR (400 MHz, DMSO-d₆) δ 12.39 (br s, 1H), 8.06 (d, J = 7.6 Hz, 1H), 7.98 (s, 1H), 7.73-7.68 (m, 1H), 7.68-7.62 (m, 1H), 7.48-7.43 (m, 1H), 7.26 (s, 1H), 6.27 (s, 1H), 5.70-5.63 (m, 1H), 5.15-4.91 (m, 1H), 4.70- 4.62 (m, 1H), 4.59-4.46 (m, 2H), 4.40-4.34 (m, 1H), 4.33- 4.26 (m, 1H), 4.26-4.15 (m, 1H), 4.15-4.09 (m, 1H), 3.82-3.76 (m, 1H), 3.75 (s, 3H), 3.70-3.61 (m, 1H), 3.35-3.24 (m, 1H), 3.02- 2.92 (m, 1H), 2.77-2.70 (m, 1H), 2.64-2.53 (m, 1H), 2.27 (s, 3H), 2.20-2.09 (m, 1H), 1.11- 0.99 (m, 2H), 0.97-0.89 (m, 2H), 0.84 (d, J = 6.4 Hz, 3H). |
| | (2S,4S)-1-(2-Cyclopropylbenzofuro[3,2- | | |
| | d]pyrimidin-4-yl)-4-((5-((1,5-dimethyl- | | |
| | 1H-pyrazol-3-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 203 | 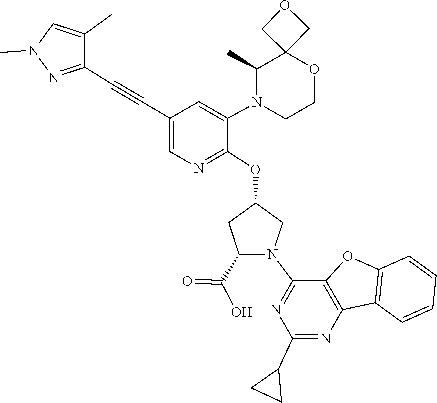 [SMILES: Cc1cn(C)nc1C#Cc1cnc(O[C@H]2C[C@@H](C(=O)O)N(c3nc(C4CC4)nc4c3oc3ccccc34)C2)c(N2CCOC3(COC3)[C@@H]2C)c1] | (2S,4S)-4-((5-((1,4- dimethyl-1H-pyrazol-3- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 28) | LCMS (ESI): mass calcd for C₃₇H₃₇N₇O₆, 675.3; m/z found, 676.3 [M + H]⁺. ¹H NMR (400 MHz, DMSO-d₆) δ 8.07 (d, J = 7.6 Hz, 1H), 8.00 (d, J = 2.0 Hz, 1H), 7.73- 7.68 (m, 1H), 7.67-7.61 (m, 1H), 7.51 (s, 1H), 7.49-7.43 (m, 1H), 7.26 (d, J = 2.0 Hz, 1H), 5.73-5.61 (m, 1H), 5.11-4.93 (m, 1H), 4.70-4.62 (m, 1H), 4.58- 4.47 (m, 2H), 4.38-4.34 (m, 1H), 4.34-4.27 (m, 1H), 4.2S-4.15 (m, 1H), 4.14-4.09 (m, 1H), 3.80 (s, 3H), 3.79-3.75 (m, 1H), 3.69- 3.63 (m, 1H), 3.32-3.31 (m, 1H), 2.95-2.92 (m, 1H), 2.75- 2.71 (m, 1H), 2.54-2.52 (m, 1H), 2.19-2.13 (m, 1H), 2.10 (s, 3H), 1.09-1.01 (m, 2H), 0.96-0.90 (m, 2H), 0.84 (d, J = 6.8 Hz, 3H). |
| | (2S,4S)-1-(2-Cyclopropylbenzofuro[3,2- | | |
| | d]pyrimidin-4-yl)-4-((5-((1,4-dimethyl- | | |
| | 1H-pyrazol-3-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 204 | 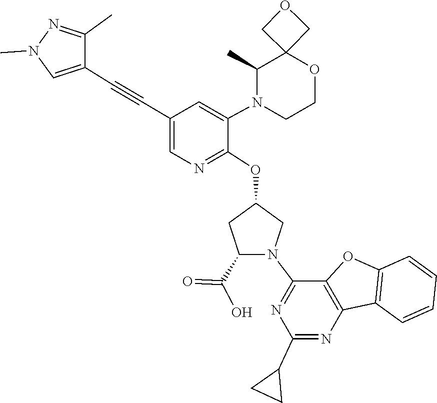 [SMILES: Cc1nn(C)cc1C#Cc1cnc(O[C@H]2C[C@@H](C(=O)O)N(c3nc(C4CC4)nc4c3oc3ccccc34)C2)c(N2CCOC3(COC3)[C@@H]2C)c1] | (2S,4S)-4-((5-((1,3- dimethyl-1H-pyrazol-4- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 26) | LCMS (ESI): mass calcd for C₃₇H₃₇N₇O₆, 675.3; m/z found, 676.4 [M + H]⁺. ¹H NMR (400 MHz, DMSO-d₆) δ 8.09 (d, J = 8.0 Hz, 1H), 7.95 (d, J = 2.0 Hz, 1H), 7.86 (s, 1H), 7.76-7.71 (m, 1H), 7.71- 7.65 (m, 1H), 7.52-7.44 (m, 1H), 7.23 (d, J = 2.0 Hz, 1H), 5.71-5.61 (m, 1H), 5.13-4.98 (m, 1H), 4.68-4.63 (m, 1H), 4.60- 4.47 (m, 2H), 4.38-4.33 (m, 1H), 4.33-4.27 (m, 1H), 4.27-4.18 (m, 1H), 4.14-4.10 (m, 1H), 3.79 (s, 3H), 3.77-3.71 (m, 1H), 3.69- 3.60 (m, 1H), 3.33-3.31 (m, 1H), 3.01-2.94 (m, 1H), 2.77- 2.67 (m, 1H), 2.58-2.53 (m, 1H), 2.25 (s, 3H), 2.21-2.14 (m, 1H), 1.14-1.02 (m, 2H), 1.01-0.94 (m, 2H), 0.84 (d, J = 6.8 Hz, 3H). |
| | (2,4S)-1-(2-Cyclopropylbenzofuro[3,2- | | |
| | d]pyrimidin-4-yl)-4-((5-((1,3-dimethyl- | | |
| | 1H-pyrazol-4-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 205 | 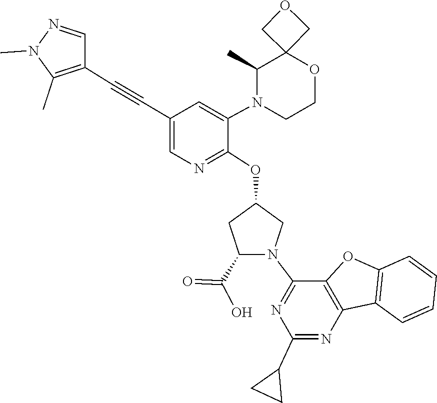 [SMILES: Cc1c(C#Cc2cnc(O[C@H]3C[C@@H](C(=O)O)N(c4nc(C5CC5)nc5c4oc4ccccc45)C3)c(N3CCOC4(COC4)[C@@H]3C)c2)cnn1C] | (2S,4S)-4-((5-((1,5- dimethyl-1H-pyrazol-4- yl)ethynyl)-3-((S)-9- methyl-2,5-dioxa-8- azaspiro[3.5]nonan-8- yl)pyridin-2- yl)oxy)pyrrolidine-2- carboxylic acid trifluoroacetate (intermediate 27) | LCMS (ESI): mass calcd for C₃₇H₃₇N₇O₆, 675.3; m/z found, 676.3 [M + H]⁺. ¹H NMR (400 MHz, DMSO-d₆) δ 8.06 (d, J = 7.6 Hz, 1H), 7.95 (d, J = 2.0 Hz, 1H), 7.73- 7.68 (m, 1H), 7.67-7.61 (m, 1H), 7.53 (s, 1H), 7.49-7.42 (m, 1H), 7.23 (d, J = 1.6 Hz, 1H), 5.69- 5.61 (m, 1H), 5.10-4.93 (m, 1H), 4.70-4.63 (m, 1H), 4.59- 4.54 (m, 1H), 4.53-4.42 (m, 1H), 4.40-4.34 (m, 1H), 4.33-4.25 (m, 1H), 4.24-4.15 (m, 1H), 4.13- 4.09 (m, 1H), 3.82-3.77 (m, 1H), 3.76 (s, 3H), 3.70-3.62 (m, 1H), 3.30-3.25 (m, 1H), 2.98- 2.94 (m, 1H), 2.74-2.68 (m, 1H), 2.53-2.52 (m, 1H), 2.37 (s, 3H), 2.19-2.11 (m, 1H), 1.09-1.00 (m, 2H), 0.96-0.89 (m, 2H), 0.84 (d, J = 6.4 Hz, 3H). |
| | (2S,4S)-1-(2-Cyclopropylbenzofuro[3,2- | | |
| | d]pyrimidin-4-yl)-4-((5-((1,5-dimethyl- | | |
| | 1H-pyrazol-4-yl)ethynyl)-3-((S)-9- | | |
| | methyl-2,5-dioxa-8-azaspiro[3.5]nonan- | | |
| | 8-yl)pyridin-2-yl)oxy)pyrrolidine-2- | | |
| | carboxylic acid | | |
| 207 | 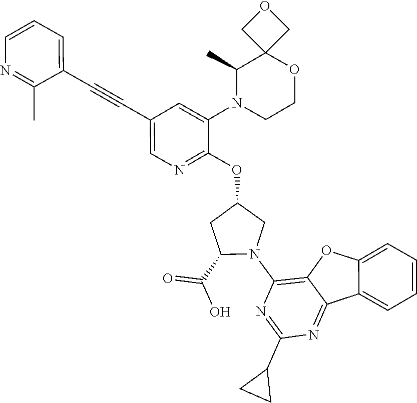 [SMILES: Cc1ncccc1C#Cc1cnc(O[C@H]2C[C@@H](C(=O)O)N(c3nc(C4CC4)nc4c3oc3ccccc34)C2)c(N2CCOC3(COC3)[C@@H]2C)c1] | (2S,4S)-4-((3-((S)-9- methyl-2,5-dioxa-8- azaspiro[3.5]nonan-8- yl)-5-((2-methylpyridin- 3-yl)ethynyl)pyridin-2- yl)oxy)pyrrolidine-2- carboxylic acid trifluoroacetate (intermediate 21) | LCMS (ESI): mass calcd for C₃₈H₃₆N₆O₆, 672.3; m/z found, 673.3 [M + H]⁺. ¹H NMR (400 MHz, DMSO-d₆) δ 8.47-8.43 (m, 1H), 8.09-8.04 (m, 2H), 7.89-7.84 (m, 1H), 7.72-7.68 (m, 1H), 7.67- 7.62 (m, 1H), 7.48-7.43 (m, 1H), 7.32 (d, J = 1.6 Hz, 1H), 7.29-7.23 (m, 1H), 5.72-5.64 (m, 1H), 5.14-4.94 (m, 1H), 4.69- 4.63 (m, 1H), 4.59-4.48 (m, 2H), 4.39-4.34 (m, 1H), 4.34-4.28 (m, 1H), 4.27-4.17 (m, 1H), 4.15- 4.10 (m, 1H), 3.81-3.74 (m, 1H), 3.70-3.62 (m, 1H), 3.31- 3.26 (m, 1H), 3.01-2.96 (m, 1H), 2.78-2.72 (m, 1H), 2.68 (s, 3H), 2.53-2.51 (m, 1H), 2.20-2.12 (m, 1H), 1.09-1.01 (m, 2H), 0.96- 0.90 (m, 2H), 0.85 (d, J = 6.4 Hz, 3H). |
| | (2S,4S)-1-(2- | | |
| | Cyclopropylbenzofuro[3,2- | | |
| | d]pyrimidin-4-yl)-4-((3-((S)-9-methyl- | | |
| | 2,5-dioxa-8-azaspiro[3.5]nonan-8-yl)- | | |
| | 5-((2-methylpyridin-3- | | |
| | yl)ethynyl)pyridin-2- | | |
| | yl)oxy)pyrrolidine-2-carboxylic acid | | |

### Example 206: (2S,4S)-1-(2-Cyclopropylbenzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid

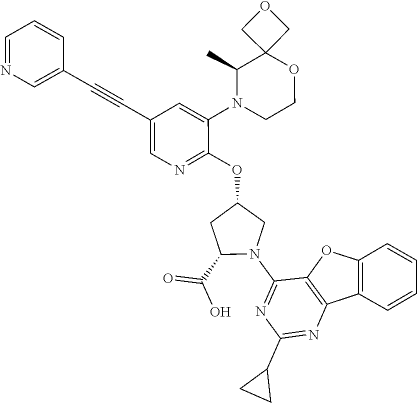 [SMILES: C[C@@H]1N(c2cc(C#Cc3cccnc3)cnc2O[C@H]2C[C@@H](C(=O)O)N(c3nc(C4CC4)nc4c3oc3ccccc34)C2)CCOC12COC2]

Step A. (S)-9-Methyl-8-(2-nitro-5-(pyridin-3-ylethynyl)pyridin-3-yl)-2,5-dioxa-8-azaspiro[3.5]nonane. The title compound was prepared in a manner analogous to Intermediate 37, using (S)-8-(5-bromo-2-nitropyridin-3-yl)-9-methyl-2,5-dioxa-8-azaspiro[3.5]nonane (Intermediate 17, product from Step A) and 3-ethynylpyridine. LCMS (ESI): mass calcd for C₁₉H₁₈N₄O₄, 366.1; m/z found, 367.1 [M+H]⁺. ¹H NMR (400 MHz, CDCl₃) δ 8.83 (d, J=1.6 Hz, 1H), 8.70-8.60 (m, 1H), 8.29 (d, J=2.0 Hz, 1H), 7.92-7.80 (m, 1H), 7.73 (d, J=1.6 Hz, 1H), 7.43-7.33 (m, 1H), 4.79-4.71 (m, 1H), 4.63-4.58 (m, 1H), 4.54-4.50 (m, 1H), 4.45-4.40 (m, 1H), 3.86-3.79 (m, 1H), 3.79-3.69 (m, 2H), 3.43-3.32 (m, 1H), 2.77-2.69 (m, 1H), 1.15 (d, J=6.4 Hz, 3H).

Step B. (2S,4S)-1-(2-Cyclopropylbenzofuro[3,2-d]pyrimidin-4-yl)-4-((3-((S)-9-methyl-2,5-dioxa-8-azaspiro[3.5]nonan-8-yl)-5-(pyridin-3-ylethynyl)pyridin-2-yl)oxy)pyrrolidine-2-carboxylic acid. A solution of (S)-9-methyl-8-(2-nitro-5-(pyridin-3-ylethynyl)pyridin-3-yl)-2,5-dioxa-8-azaspiro[3.5]nonane (50.0 mg, 0.133 mmol), (2S,4S)-1-(2-cyclopropylbenzofuro[3,2-d]pyrimidin-4-yl)-4-hydroxypyrrolidine-2-carboxylic acid (Intermediate 38, 60.0 mg, 0.177 mmol) and DMF (2.00 mL) was subsequently subjected to three cycles of vacuum and recharging with nitrogen and cooled to 0° C. (ice/water). NaH (32.0 mg, 0.800 mmol, 60% in mineral oil) was added to the reaction mixture in portions over 5 min and the reaction mixture was removed from the ice bath and stirred at rt for 2 h. The reaction mixture was treated with ice water (1 mL) and passed through a syringe filter (0.45 μm nylon membrane). Purification (HPLC (Phenomenex Gemini NX C18, 5 μm, 150 mm×30 mm; 7 min gradient (28-58% ACN/H₂O (with 0.2% FA)) at 25 mL/min)) afforded the title compound as a white powder (54.4 mg, 61%). LCMS (ESI): mass calcd for C₃₇H₃₄N₆O₆, 658.3; r/z found, 659.3 [M+H]⁺. ¹H NMR (400 MHz, DMSO-d₆) δ 8.75 (s, 1H), 8.58 (d, J=4.8 Hz, 1H), 8.10-8.02 (m, 2H), 7.95 (d, J=7.2 Hz, 1H), 7.74-7.67 (m, 1H), 7.67-7.60 (m, 1H), 7.49-7.42 (m, 2H), 7.34 (s, 1H), 5.73-5.61 (m, 1H), 5.11-4.92 (m, 1H), 4.70-4.62 (m, 1H), 4.60-4.44 (m, 2H), 4.40-4.27 (m, 2H), 4.27-4.14 (m, 1H), 4.14-4.09 (m, 1H), 3.83-3.73 (m, 1H), 3.71-3.60 (m, 1H), 3.33-3.26 (m, 1H), 2.98-2.93 (m, 1H), 2.78-2.71 (m, 1H), 2.57-2.51 (m, 1H), 2.21-2.09 (m, 1H), 1.11-0.99 (m, 2H), 0.97-0.89 (m, 2H), 0.85 (d, J=6.4 Hz, 3H).

Examples 208-218, in Table 20 were prepared in a manner analogous to Example 37, using 2S,4S)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid trifluoroacetate and corresponding halide or trifluoromethanesulfonate. Table 20.

TABLE 20

| | | Halide or | |
|---|---|---|---|
| | | trifluoromethane- | |
| Ex # | Structure | sulfonate | MS and 1H-NMR |
| 208 | 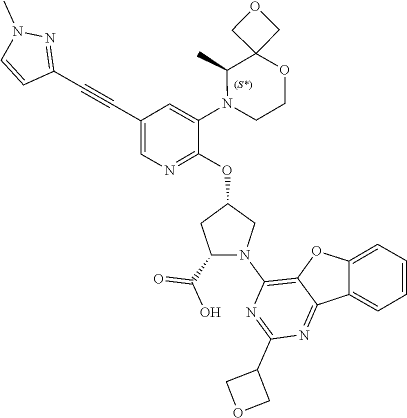 [SMILES: C[C@@H]1N(c2cc(C#Cc3ccn(C)n3)cnc2O[C@H]2C[C@@H](C(=O)O)N(c3nc(C4COC4)nc4c3oc3ccccc34)C2)CCOC12COC2] | 2-(oxetan-3- yl)benzofuro[3,2- d]pyrimidin-4-yl trifluoromethane- sulfonate (Intermediate 56) | LCMS (ESI): mass calcd for C₃₆H₃₅N₇O₇, 677.3; m/z found, 678.2 [M + H]⁺. ¹H NMR (400 MHz, DMSO-d₆) δ 8.10 (d, J = 7.6 Hz, 1H), 8.00 (s, 1H), 7.78-7.65 (m, 3H), 7.53-7.45 (m, 1H), 7.28 (s, 1H), 6.48 (s, 1H), 5.73-5.64 (m, 1H), 5.29-5.07 (m, 1H), 5.02- 4.87 (m, 4H), 4.69-4.63 (m, 1H), 4.62-4.50 (m, 2H), 4.49-4.37 (m, 1H), 4.37-4.22 (m, 3H), 4.14- 4.07 (m, 1H), 3.87 (s, 3H), 3.81- 3.73 (m, 1H), 3.70-3.60 (m, 1H), 3.33-3.31 (m, 1H), 3.06-2.97 (m, 1H), 2.78-2.69 (m, 1H), 2.65- 2.55 (m, 1H), 0.84 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-(oxetan-3-yl)benzofuro[3,2- | | |
| | d]pyrimidin-4-yl)pyrrolidine-2-carboxylic | | |
| | acid | | |
| 209 | 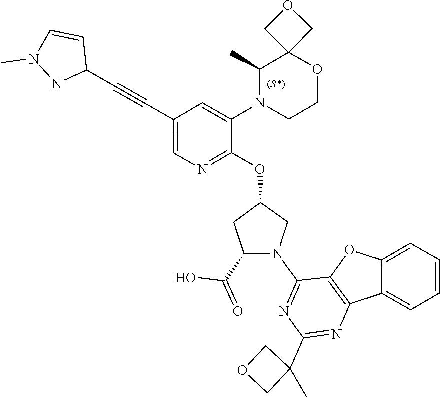 [SMILES: C[C@@H]1N(c2cc(C#CC3C=CN(C)N3)cnc2O[C@H]2C[C@@H](C(=O)O)N(c3nc(C4(C)COC4)nc4c3oc3ccccc34)C2)CCOC12COC2] | 2-(3-methyloxetan-3- yl)benzofuro[3,2- d]pyrimidin-4-yl trifluoromethane- sulfonate (Intermediate 55) | LCMS (ESI): mass calcd for C₃₇H₃₇N₇O₇, 691.3; m/z found, 692.3 [M + H]⁺. ¹H NMR (400 MHz, DMSO-d₆) δ 12.39 (br s, 1H), 8.10 (d, J = 8.0 Hz, 1H), 8.00 (d, J = 1.2 Hz, 1H), 7.79-7.71 (m, 2H), 7.70- 7.64 (m, 1H), 7.52-7.44 (m, 1H), 7.27 (s, 1H), 6.48 (d, J = 1.6 Hz, 1H), 5.68 (br s, 1H), 5.10 (t, J = 6.0 Hz, 2H), 4.69-4.63 (m, 1H), 4.59- 4.46 (m, 4H), 4.38-4.21 (m, 3H), 4.13-4.07 (m, 1H), 3.87 (s, 3H), 3.80-3.72 (m, 1H), 3.70-3.61 (m, 1H), 3.34-3.24 (m, 1H), 3.05- 2.93 (m, 2H), 2.76-2.69 (m, 1H), 2.59-2.53 (m, 1H), 1.75 (s, 3H), 0.84 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-(3-methyloxetan-3- | | |
| | yl)benzofuro[3,2-d]pyrimidin-4- | | |
| | yl)pyrrolidine-2-carboxylic acid | | |
| 210 | 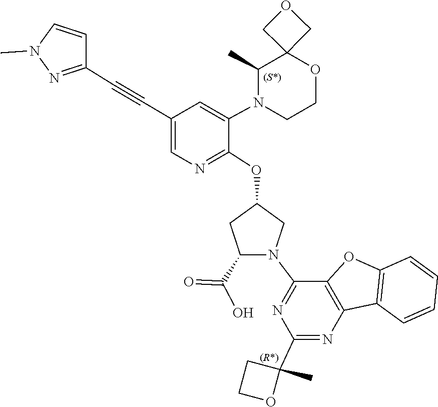 [SMILES: C[C@@H]1N(c2cc(C#Cc3ccn(C)n3)cnc2O[C@H]2C[C@@H](C(=O)O)N(c3nc([C@@]4(C)CCO4)nc4c3oc3ccccc34)C2)CCOC12COC2] | 2-(2-Methyloxetan-2- yl)benzofuro[3,2- d]pyrimidin-4-yl trifluoromethane- sulfonate (Intermediate 50) | LCMS (ESI): mass calcd for C₃₇H₃₇N₇O₇, 691.3; m/z found, 692.3 [M + H]⁺. ¹H NMR (400 MHz, DMSO-d₆) δ 8.12 (d, J = 7.6 Hz, 1H), 8.00 (d, J = 1.6 Hz, 1H), 7.79-7.72 (m, 2H), 7.71-7.65 (m, 1H), 7.54-7.45 (m, 1H), 7.28 (d, J = 1.2 Hz, 1H), 6.48 (d, J = 2.0 Hz, 1H), 5.75- 5.65 (m, 1H), 4.70-4.64 (m, 1H), 4.62-4.46 (m, 4H), 4.38-4.22 (m, 3H), 4.13-4.07 (m, 1H), 3.87 (s, 3H), 3.81-3.74 (m, 1H), 3.70- 3.61 (m, 1H), 3.43-3.25 (m, 4H), 2.78-2.70 (m, 1H), 2.69-2.56 (m, 2H), 1.81 (s, 3H), 0.84 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((R*)-2-methyloxetan-2- | | |
| | yl)benzofuro[3,2-d]pyrimidin-4- | | |
| | yl)pyrrolidine-2-carboxylic acid | | |
| 211 | 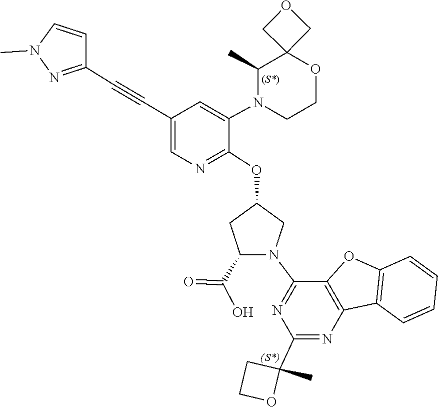 [SMILES: C[C@@H]1N(c2cc(C#Cc3ccn(C)n3)cnc2O[C@H]2C[C@@H](C(=O)O)N(c3nc([C@@]4(C)CCO4)nc4c3oc3ccccc34)C2)CCOC12COC2] | 2-(2-Methyloxetan-2- yl)benzofuro[3,2- d]pyrimidin-4-yl trifluoromethane- sulfonate (Intermediate 50) | LCMS (ESI): mass calcd for C₃₇H₃₇N₇O₇, 691.3; m/z found, 692.3 [M + H]⁺. ¹H NMR (400 MHz, DMSO-d₆) δ 8.12 (d, J = 7.6 Hz, 1H), 8.00 (d, J = 2.0 Hz, 1H), 7.81- 7.63 (m, 3H), 7.53-7.46 (m, 1H), 7.27 (d, J = 1.6 Hz, 1H), 6.48 (d, J = 2.0 Hz, 1H), 5.83-5.62 (m, 1H), 4.69-4.63 (m, 1H), 4.61-4.48 (m, 4H), 4.39-4.25 (m, 3H), 4.13- 4.08 (m, 1H), 3.87 (s, 3H), 3.80- 3.74 (m, 1H), 3.70-3.62 (m, 1H), 3.34-3.24 (m, 4H), 2.76-2.70 (m, 1H), 2.70-2.59 (m, 2H), 1.82 (s, 3H), 0.84 (d, J = 6.8 Hz, 3H) |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((S*)-2-methyloxetan-2- | | |
| | yl)benzofuro[3,2-d]pyrimidin-4- | | |
| | yl)pyrrolidine-2-carboxylic acid | | |
| 212 | 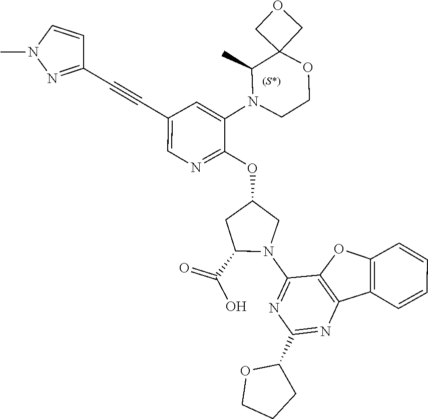 [SMILES: C[C@@H]1N(c2cc(C#Cc3ccn(C)n3)cnc2O[C@H]2C[C@@H](C(=O)O)N(c3nc([C@@H]4CCCO4)nc4c3oc3ccccc34)C2)CCOC12COC2] | (S)-4-chloro-2- (tetrahydrofuran-2- yl)benzofuro[3,2- d]pyrimidine (Intermediate 53) | LCMS (ESI): mass calcd for C₃₇H₃₇N₇O7, 691.3; m/z found, 692.2 [M + H]⁺. ¹H NMR (400 MHz, DMSO-d₆) δ 12.71-12.18 (m, 1H), 8.10 (d, J = 7.6 Hz, 1H), 8.01 (s, 1H), 7.78-7.72 (m, 2H), 7.71- 7.64 (m, 1H), 7.54-7.45 (m, 1H), 7.29 (s, 1H), 6.49 (d, J = 2.0 Hz, 1H), 5.75-5.65 (m, 1H), 5.18- 5.00 (m, 1H), 5.00-4.92 (m, 1H), 4.66 (d, J = 6.4 Hz, 1H), 4.63- 4.57 (m, 1H), 4.55 (d, J = 6.4 Hz, 1H), 4.36 (d, J = 6.8 Hz, 1H), 4.33- 4.17 (m, 2H), 4.13 (d, J = 6.8 Hz, 1H), 4.11-4.04 (m, 1H), 3.92- 3.84 (m, 4H), 3.82-3.73 (m, 1H), 3.70-3.60 (m, 1H), 3.36-3.25 (m, 1H), 3.03-2.96 (m, 1H), 2.79- 2.71 (m, 1H), 2.60-2.54 (m, 1H), 2.29-2.09 (m, 3H), 2.01- 1.88 (m, 1H), 0.86 (d, J = 6.8 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((S)-tetrahydrofuran-2- | | |
| | yl)benzofuro[3,2-d]pyrimidin-4- | | |
| | yl)pyrrolidine-2-carboxylic acid | | |
| 213 | 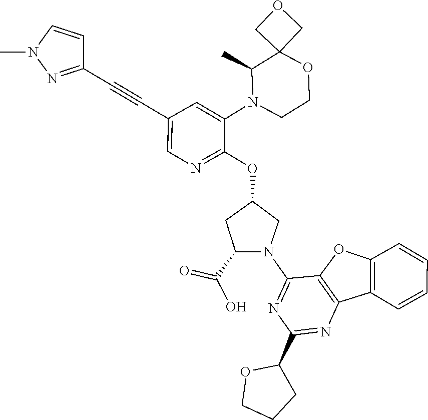 [SMILES: C[C@@H]1N(c2cc(C#Cc3ccn(C)n3)cnc2O[C@H]2C[C@@H](C(=O)O)N(c3nc([C@H]4CCCO4)nc4c3oc3ccccc34)C2)CCOC12COC2] | (R)-4-chloro-2- (tetrahydrofuran-2- yl)benzofuro[3,2- d]pyrimidine (Intermediate 54) | LCMS (ESI): mass calcd for C₃₇H₃₇N₇O₇, 691.3; m/z found, 692.3 [M + H]⁺. ¹H NMR (400 MHz, DMSO-d₆) δ 8.16-8.06 (m, 1H), 8.03-7.97 (m, 1H), 7.77-7.70 (m, 2H), 7.70-7.62 (m, 1H), 7.53- 7.43 (m, 1H), 7.30-7.23 (m, 1H), 6.53-6.45 (m, 1H), 5.72- 5.61 (m, 1H), 5.24-5.02 (m, 1H), 5.00-4.90 (m, 1H), 4.72-4.61 (m, 1H), 4.59-4.47 (m, 2H), 4.38- 4.33 (m, 1H), 4.32-4.15 (m, 2H), 4.14-4.03 (m, 2H), 3.92- 3.82 (m, 4H), 3.81-3.72 (m, 1H), 3.72-3.60 (m, 1H), 3.32-3.25 (m, 1H), 3.01-2.95 (m, 1H), 2.79- 2.68 (m, 1H), 2.62-2.55 (m, 1H), 2.28-2.08 (m, 3H), 2.02- 1.85 (m, 1H), 0.84 (d, J = 6.0 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((R)-tetrahydrofuran-2- | | |
| | yl)benzofuro[3,2-d]pyrimidin-4- | | |
| | yl)pyrrolidine-2-carboxylic acid. | | |
| 214 | 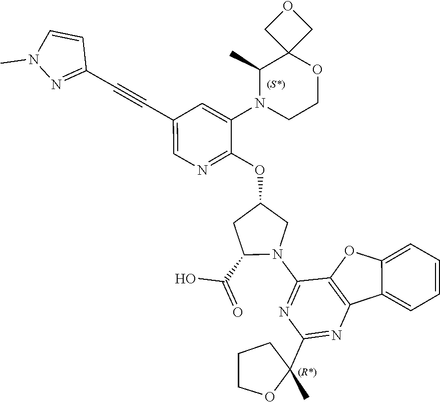 [SMILES: C[C@@H]1N(c2cc(C#Cc3ccn(C)n3)cnc2O[C@H]2C[C@@H](C(=O)O)N(c3nc([C@@]4(C)CCCO4)nc4c3oc3ccccc34)C2)CCOC12COC2] | 4-chloro-2-(2- methyltetrahydrofuran- 2-yl)benzofuro[3,2- d]pyrimidine (Intermediate 57) | LCMS (ESI): mass calcd for C₃₈H₃₉N₇O₇, 705.3; m/z found, 706.3 [M + H]⁺. ¹H NMR (400 MHz, DMSO-d₆) δ 8.09 (d, J = 7.6 Hz, 1H), 8.00 (d, J = 1.6 Hz, 1H), 7.76-7.69 (m, 2H), 7.69-7.62 (m, 1H), 7.51-7.44 (m, 1H), 7.27 (d, J = 1.2 Hz, 1H), 6.48 (d, J = 2.4 Hz, 1H), 5.71- 5.61 (m, 1H), 5.19-4.98 (m, 1H), 4.69-4.63 (m, 1H), 4.61-4.45 (m, 2H), 4.40-4.16 (m, 3H), 4.13- 4.07 (m, 1H), 3.99-3.89 (m, 2H), 3.87 (s, 3H), 3.81-3.73 (m, 1H), 3.70-3.61 (m, 1H), 3.35- 3.24 (m, 2H), 2.97-2.93 (m, 1H), 2.75-2.69 (m, 1H), 2.67-2.62 (m, 1H), 2.02-1.94 (m, 2H), 1.90- 1.79 (m, 1H), 1.64 (s, 3H), 0.84 (d, J = 6.4 Hz, 3H) |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((R*)-2- | | |
| | methyltetrahydrofuran-2- | | |
| | yl)benzofuro[3,2-d]pyrimidin-4- | | |
| | yl)pyrrolidine-2-carboxylic acid | | |
| 215 | 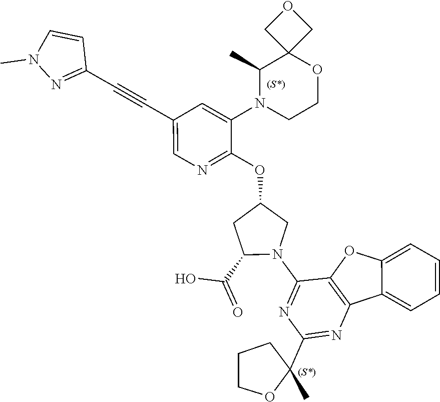 [SMILES: C[C@@H]1N(c2cc(C#Cc3ccn(C)n3)cnc2O[C@H]2C[C@@H](C(=O)O)N(c3nc([C@@]4(C)CCCO4)nc4c3oc3ccccc34)C2)CCOC12COC2] | 4-chloro-2-(2- methyltetrahydrofuran- 2-yl)benzofuro[3,2- d]pyrimidine (Intermediate 57) | LCMS (ESI): mass calcd for C₃₈H₃₉N₇O₇, 705.3; m/z found, 706.3 [M + H]⁺. ¹H NMR (400 MHz, DMSO-d₆) δ 8.15 (d, J = 7.6 Hz, 1H), 8.04 (d, J = 1.6 Hz, 1H), 7.80-7.75 (m, 2H), 7.74-7.69 (m, 1H), 7.55-7.49 (m, 1H), 7.31 (d, J = 2.0 Hz, 1H), 6.51 (d, J = 2.4 Hz, 1H), 5.78- 5.66 (m, 1H), 5.23-5.03 (m, 1H), 4.72-4.66 (m, 1H), 4.65-4.51 (m, 2H), 4.41-4.24 (m, 3H), 4.17- 4.11 (m, 1H), 4.06-3.93 (m, 2H), 3.91 (s, 3H), 3.83-3.76 (m, 1H), 3.74-3.64 (m, 1H), 3.38- 3.26 (m, 2H), 3.04-2.99 (m, 1H), 2.80-2.72 (m, 1H), 2.66-2.62 (m, 1H), 2.06-1.98 (m, 2H), 1.95- 1.87 (m, 1H), 1.67 (s, 3H), 0.87 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((S*)-2- | | |
| | methyltetrahydrofuran-2- | | |
| | yl)benzofuro[3,2-d]pyrimidin-4- | | |
| | yl)pyrrolidine-2-carboxylic acid | | |
| 216 | 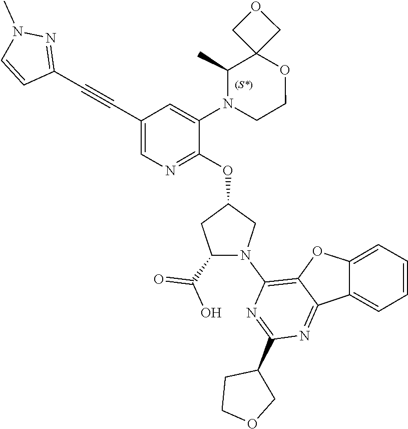 [SMILES: C[C@@H]1N(c2cc(C#Cc3ccn(C)n3)cnc2O[C@H]2C[C@@H](C(=O)O)N(c3nc([C@@H]4CCOC4)nc4c3oc3ccccc34)C2)CCOC12COC2] | (S)-4-chloro-2- (tetrahydrofuran-3- yl)benzofuro[3,2- d]pyrimidine (Intermediate 52) | LCMS (ESI): mass calcd for C₃₇H₃₇N₇O₇, 691.3; m/z found, 692.3 [M + H]⁺. ¹H NMR (400 MHz, DMSO-d₆) δ 8.08 (d, J = 8.0 Hz, 1H), 8.00 (d, J = 1.2 Hz, 1H), 7.74-7.68 (m, 2H), 7.68-7.61 (m, 1H), 7.50-7.43 (m, 1H), 7.27 (d, J = 1.2 Hz, 1H), 6.48 (d, J = 2.0 Hz, 1H), 5.70- 5.60 (m, 1H), 5.19-4.93 (m, 1H), 4.70-4.62 (m, 1H), 4.61-4.46 (m, 2H), 4.41-4.28 (m, 2H), 4.26- 4.15 (m, 1H), 4.14-4.08 (m, 2H), 3.98-3.89 (m, 2H), 3.87 (s, 3H), 3.86-3.81 (m, 1H), 3.80- 3.73 (m, 1H), 3.70-3.57 (m, 2H), 3.31-3.25 (m, 1H), 3.02-2.93 (m, 2H), 2.77-2.69 (m, 1H), 2.43- 2.35 (m, 1H), 2.30-2.20 (m, 1H), 0.83 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((S)-tetrahydrofuran-3- | | |
| | yl)benzofuro[3,2-d]pyrimidin-4- | | |
| | yl)pyrrolidine-2-carboxylic acid | | |
| 217 | 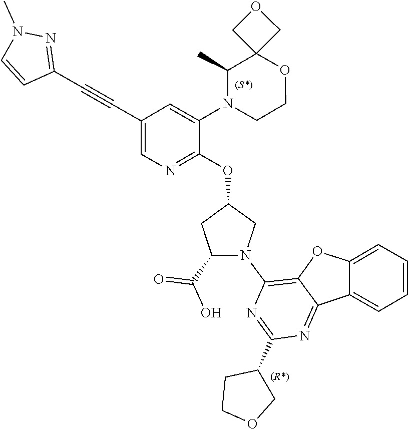 [SMILES: C[C@@H]1N(c2cc(C#Cc3ccn(C)n3)cnc2O[C@H]2C[C@@H](C(=O)O)N(c3nc([C@H]4CCOC4)nc4c3oc3ccccc34)C2)CCOC12COC2] | (R)-4-chloro-2- (tetrahydrofuran-3- yl)benzofuro[3,2- d]pyrimidine (Intermediate 51) | LCMS (ESI): mass calcd for C₃₇H₃₇N₇O₇, 691.3; m/z found, 692.3 [M + H]⁺. ¹H NMR (400 MHz, DMSO-d₆) δ 8.08 (d, J = 7.6 Hz, 1H), 8.02- 7.97 (m, 1H), 7.75-7.70 (m, 2H), 7.69-7.63 (m, 1H), 7.50-7.44 (m, 1H), 7.30-7.24 (m, 1H), 6.50- 6.46 (m, 1H), 5.71-5.63 (m, 1H), 5.18-4.95 (m, 1H), 4.70- 4.62 (m, 1H), 4.61-4.46 (m, 2H), 4.39-4.28 (m, 2H), 4.27-4.17 (m, 1H), 4.15-4.07 (m, 2H), 3.98- 3.89 (m, 2H), 3.87 (s, 3H), 3.86- 3.81 (m, 1H), 3.80-3.73 (m, 1H), 3.71-3.57 (m, 2H), 3.33-3.24 (m, 1H), 3.05-2.93 (m, 2H), 2.77- 2.70 (m, 1H), 2.43-2.33 (m, 1H), 2.30-2.18 (m, 1H), 0.84 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-((R*)-tetrahydrofuran-3- | | |
| | yl)benzofuro[3,2-d]pyrimidin-4- | | |
| | yl)pyrrolidine-2-carboxylic acid | | |
| 218 | 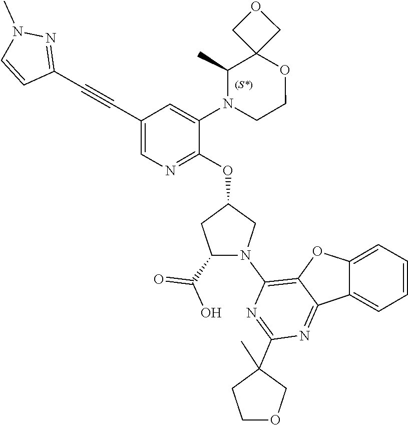 [SMILES: C[C@@H]1N(c2cc(C#Cc3ccn(C)n3)cnc2O[C@H]2C[C@@H](C(=O)O)N(c3nc(C4(C)CCOC4)nc4c3oc3ccccc34)C2)CCOC12COC2] | 4-chloro-2-(3- methyltetrahydrofuran- 3-yl)benzofuro[3,2- d]pyrimidine (Intermediate 48) | LCMS (ESI): mass calcd for C₃₈H₃₉N₇O₇, 705.3; m/z found, 706.3 [M + H]⁺. ¹H NMR (400 MHz, DMSO-d₆) δ 8.09 (d, J = 7.6 Hz, 1H), 8.01 (d, J = 1.6 Hz, 1H), 7.73 (br d, J = 2.0 Hz, 2H), 7.69-7.63 (m, 1H), 7.52-7.43 (m, 1H), 7.28 (d, J = 1.6 Hz, 1H), 6.49 (d, J = 2.0 Hz, 1H), 5.71-5.62 (m, 1H), 5.14- 4.97 (m, 1H), 4.67 (d, J = 6.8 Hz, 1H), 4.55 (d, J = 6.4 Hz, 1H), 4.39- 4.31 (m, 2H), 4.31-4.21 (m, 2H), 4.11 (d, J = 6.8 Hz, 1H), 3.97- 3.90 (m, 1H), 3.88 (s, 3H), 3.87- 3.82 (m, 1H), 3.81-3.74 (m, 1H), 3.71-3.68 (m, 1H), 3.68-3.62 (m, 1H), 3.36-3.25 (m, 2H), 3.01- 2.95 (m, 2H), 2.84-2.77 (m, 1H), 2.76-2.70 (m, 1H), 1.98- 1.88 (m, 1H), 1.50 (d, J = 2.0 Hz, 3H), 0.84 (d, J = 6.4 Hz, 3H). |
| | (2S,4S)-4-((5-((1-Methyl-1H-pyrazol-3- | | |
| | yl)ethynyl)-3-((S*)-9-methyl-2,5-dioxa-8- | | |
| | azaspiro[3.5]nonan-8-yl)pyridin-2- | | |
| | yl)oxy)-1-(2-(3-methyltetrahydrofuran- | | |
| | 3-yl)benzofuro[3,2-d]pyrimidin-4- | | |
| | yl)pyrrolidine-2-carboxylic acid | | |

### Example 219: (2S,4S)-1-(2-(3,3-Difluoroazetidin-1-yl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

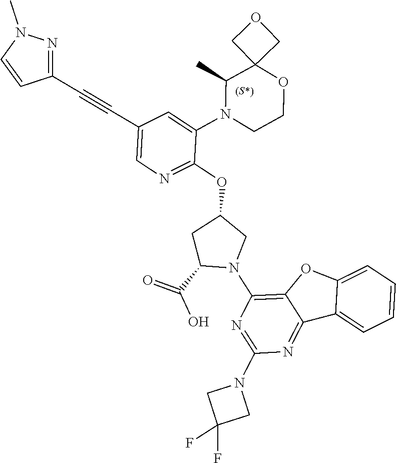 [SMILES: C[C@@H]1N(c2cc(C#Cc3ccn(C)n3)cnc2O[C@H]2C[C@@H](C(=O)O)N(c3nc(N4CC(F)(F)C4)nc4c3oc3ccccc34)C2)CCOC12COC2]

A mixture of (S*)-9-methyl-8-(5-((1-methyl-1H-pyrazol-3-yl)ethynyl)-2-nitropyridin-3-yl)-2,5-dioxa-8-azaspiro[3.5]nonane (Intermediate 58, 30.0 mg, 0.0800 mmol), (2S,4S)-1-(2-(3,3-difluoroazetidin-1-yl)benzofuro[3,2-d]pyrimidin-4-yl)-4-hydroxypyrrolidine-2-carboxylic acid (Intermediate 60, 42.0 mg, 0.0973 mmol), DMF (2.00 mL), and NaH (60% in mineral oil, 12.0 mg, 0.500 mmol) were combined at 0° C. (ice/water). The reaction mixture was subsequently subjected to three cycles of vacuum and recharging with nitrogen, the reaction mixture was allowed to warm to rt and stirred at rt for 2 h. The reaction mixture was quenched with ice-water (5 mL) at 0° C., treated with saturated aq. citric acid until the pH measured 6. The reaction mixture was extracted with ethyl acetate (10 mL×3) and the combined extracts were concentrated to dryness in vacuo to give a brown oil. The resulting residue was purified (HPLC (Welch Xtimate C18, 5 μm, 150 mm×30 mm; 7 min gradient (44-74% MeCN/H₂O (with 0.02% FA)) at 25 mL/min)) to afford the title compound, (2S,4S)-1-(2-(3,3-difluoroazetidin-1-yl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid as white powder (32.8 mg, 56%). LCMS (ESI): mass calcd for C₃₆H₃₄F₂N₈O₆, 712.3; m/z found, 713.2 [M+H]⁺. ¹H NMR (400 MHz, DMSO-d₆) d=8.02-7.97 (m, 2H), 7.76-7.70 (m, 1H), 7.68-7.59 (m, 2H), 7.45-7.37 (m, 1H), 7.28 (d, J=1.6 Hz, 1H), 6.48 (d, J=2.4 Hz, 1H), 5.73-5.61 (m, 1H), 5.27-4.84 (m, 1H), 4.66 (d, J=6.8 Hz, 1H), 4.60-4.49 (m, 2H), 4.48-4.39 (m, 4H), 4.36 (d, J=6.8 Hz, 1H), 4.34-4.27 (m, 1H), 4.25-4.10 (m, 2H), 3.87 (s, 3H), 3.81-3.73 (m, 1H), 3.70-3.60 (m, 1H), 3.34-3.23 (m, 1H), 3.04-2.92 (m, 2H), 2.73 (br d, J=12.8 Hz, 1H), 0.85 (d, J=6.8 Hz, 3H).

### Example 220: (2S,4S)-4-((5-((1-Methyl-1H-pyrazol-3-yl)ethynyl)-3-((S*)-9-methyl-2,5-dioxa-8-azaspiro[3.5]nonan-8-yl)pyridin-2-yl)oxy)-1-(2-(pyrrolidin-1-yl)benzofuro[3,2-d]pyrimidin-4-yl)pyrrolidine-2-carboxylic acid

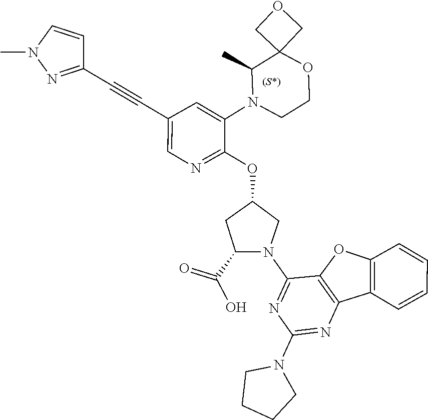 [SMILES: C[C@@H]1N(c2cc(C#Cc3ccn(C)n3)cnc2O[C@H]2C[C@@H](C(=O)O)N(c3nc(N4CCCC4)nc4c3oc3ccccc34)C2)CCOC12COC2]

The title compound was prepared in a manner analogous to 221, using (S*)-9-methyl-8-(5-((1-methyl-1H-pyrazol-3-yl)ethynyl)-2-nitropyridin-3-yl)-2,5-dioxa-8-azaspiro[3.5]nonane (Intermediate 58) and (2S,4S)-4-hydroxy-1-(2-(pyrrolidin-1-yl)benzofuro[3,2-d]pyrimidin-4-yl)pyrrolidine-2-carboxylic acid (Intermediate 45). LCMS (ESI): mass calcd for C₃₇H₃₈N₈O₆, 690.3; m/z found, 691.3 [M+H]⁺. ¹H NMR (400 MHz, DMSO-d₆) δ 8.00 (d, J=2.0 Hz, 1H), 7.95 (d, J=7.6 Hz, 1H), 7.72 (d, J=2.0 Hz, 1H), 7.63-7.59 (m, 1H), 7.59-7.53 (m, 1H), 7.40-7.34 (m, 1H), 7.27 (d, J=2.0 Hz, 1H), 6.48 (d, J=2.0 Hz, 1H), 5.71-5.61 (m, 1H), 5.06-4.90 (m, 1H), 4.71-4.65 (m, 1H), 4.60-4.51 (m, 2H), 4.42-4.36 (m, 1H), 4.34-4.27 (m, 1H), 4.19-4.10 (m, 2H), 3.87 (s, 3H), 3.80-3.75 (m, 1H), 3.70-3.63 (m, 1H), 3.58-3.53 (m, 4H), 3.33-3.28 (m, 2H), 2.95-2.91 (m, 1H), 2.76-2.71 (m, 1H), 1.97-1.88 (m, 4H), 0.85 (d, J=6.4 Hz, 3H).

### Example 221: (2S,4S)-1-(2-(4,4-Difluoropiperidin-1-yl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid

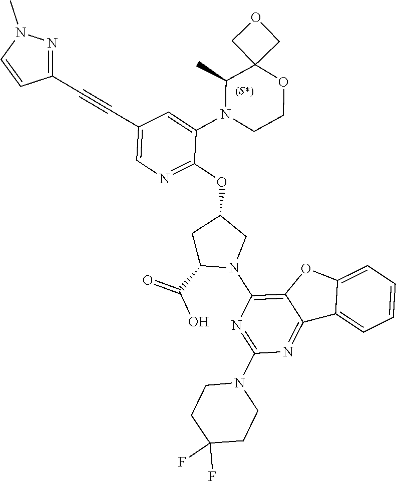 [SMILES: C[C@@H]1N(c2cc(C#Cc3ccn(C)n3)cnc2O[C@H]2C[C@@H](C(=O)O)N(c3nc(N4CCC(F)(F)CC4)nc4c3oc3ccccc34)C2)CCOC12COC2]

A mixture of (S)-9-Methyl-8-(5-((1-methyl-1H-pyrazol-3-yl)ethynyl)-2-nitropyridin-3-yl)-2,5-dioxa-8-azaspiro[3.5]nonane (Intermediate 58, 30.0 mg, 0.0793 mmol), (2S,4S)-1-(2-(4,4-difluoropiperidin-1-yl)benzofuro[3,2-d]pyrimidin-4-yl)-4-hydroxypyrrolidine-2-carboxylic acid (Intermediate 44, 55.0 mg, 0.131 mmol), and DMF (1.50 mL) was subsequently subjected to three cycles of vacuum and recharging with nitrogen and cooled to 0° C. (ice/water). NaH (18.0 mg, 0.450 mmol, 60% in mineral oil) was added and the reaction mixture was removed from the ice bath and stirred for 2 h. The reaction mixture was cooled to 0° C., treated with ice water (1 mL) and directly purified (HPLC:Welch Xtimate C18, 5 μm, 150 mm×30 mm; 7 min gradient (44-74% ACN/H₂O (with 0.2% FA)) at 25 mL/min) to afford the title compound, (2S,4S)-1-(2-(4,4-difluoropiperidin-1-yl)benzofuro[3,2-d]pyrimidin-4-yl)-4-((5-((1-methyl-1H-pyrazol-3-yl)ethynyl)-3-((S*)-9-methyl-2,5-dioxa-8-azaspiro[3.5]nonan-8-yl)pyridin-2-yl)oxy)pyrrolidine-2-carboxylic acid as a white powder (40.2 mg, 67%). LCMS (ESI): mass calcd for C₃₈H₃₈F₂N₈O₆, 740.3; m/z found, 741.3 [M+H]⁺. ¹H NMR (400 MHz, DMSO-d₆) δ 8.01-7.99 (m, 1H), 7.98 (d, J=8.0 Hz, 1H), 7.74-7.71 (m, 1H), 7.66-7.56 (m, 2H), 7.44-7.35 (m, 1H), 7.27 (s, 1H), 6.48 (d, J=2.4 Hz, 1H), 5.70-5.63 (m, 1H), 5.08-4.85 (m, 1H), 4.72-4.62 (m, 1H), 4.62-4.44 (m, 2H), 4.40-4.36 (m, 1H), 4.36-4.28 (m, 1H), 4.24-4.15 (m, 1H), 4.15-4.09 (m, 1H), 3.97-3.89 (m, 4H), 3.87 (s, 3H), 3.81-3.73 (m, 1H), 3.69-3.62 (m, 1H), 3.33-3.28 (m, 2H), 2.97-2.94 (m, 1H), 2.74-2.70 (m, 1H), 2.08-1.91 (m, 4H), 0.84 (d, J=6.8 Hz, 3H).

### Example 222: (2S,4S)-4-((5-((1-Methyl-1H-pyrazol-3-yl)ethynyl)-3-((S*)-9-methyl-2,5-dioxa-8-azaspiro[3.5]nonan-8-yl)pyridin-2-yl)oxy)-1-(2-(4-methylpiperazin-1-yl)benzofuro[3,2-d]pyrimidin-4-yl)pyrrolidine-2-carboxylic acid

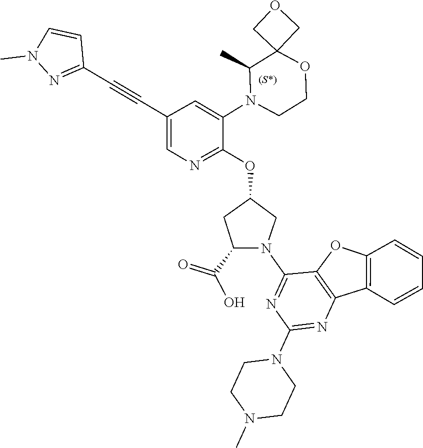 [SMILES: C[C@@H]1N(c2cc(C#Cc3ccn(C)n3)cnc2O[C@H]2C[C@@H](C(=O)O)N(c3nc(N4CCN(C)CC4)nc4c3oc3ccccc34)C2)CCOC12COC2]

The title compound was prepared in a manner analogous to Example 221, using (S*)-9-methyl-8-(5-((1-methyl-1H-pyrazol-3-yl)ethynyl)-2-nitropyridin-3-yl)-2,5-dioxa-8-azaspiro[3.5]nonane (Intermediate 58) and (2S,4S)-4-hydroxy-1-(2-(4-methylpiperazin-1-yl)benzofuro[3,2-d]pyrimidin-4-yl)pyrrolidine-2-carboxylic acid (Intermediate 59). LCMS (ESI): mass calcd for C₃₈H₄₁N₉O₆, 719.3; m/z found, 720.2 [M+H]⁺. ¹H NMR (400 MHz, DMSO-d₆) δ 8.01-7.98 (m, 1H), 7.98-7.93 (m, 1H), 7.75-7.69 (m, 1H), 7.66-7.54 (m, 2H), 7.42-7.35 (m, 1H), 7.31-7.24 (m, 1H), 6.50-6.46 (m, 1H), 5.74-5.61 (m, 1H), 5.14-4.91 (m, 1H), 4.73-4.61 (m, 1H), 4.60-4.48 (m, 2H), 4.42-4.34 (m, 1H), 4.34-4.26 (m, 1H), 4.23-4.10 (m, 2H), 3.93-3.81 (m, 3H), 3.80-3.72 (m, 5H), 3.70-3.61 (m, 1H), 3.31-3.27 (m, 1H), 3.02-2.96 (m, 2H), 2.76-2.71 (m, 1H), 2.43-2.37 (m, 4H), 2.28-2.21 (m, 3H), 0.85 (br d, J=6.0 Hz, 3H).

Examples 223-229 in Table 21 were prepared in a manner analogous to Example 1, using the appropriate alkyne and the corresponding halide under Sonogashira coupling conditions with a catalyst and ligand such as XPhos Pd G3 or 2nd Generation XPhos & XPhos, a base such as Cs₂CO₃, in CH₃CN or DMF, at temperatures ranging from rt to 100° C. for period of 1 to 24 hours.

TABLE 21

| Ex # | Structure | Alkyne / Halide | MS and 1H NMR |
|---|---|---|---|
| 223 | 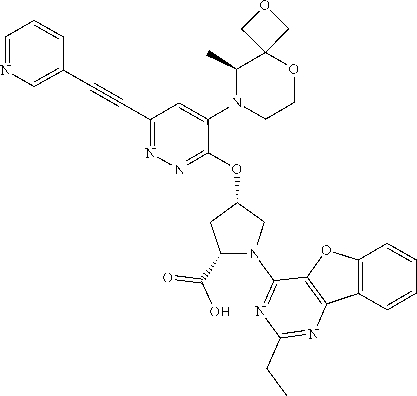 [SMILES: CCc1nc(N2C[C@@H](Oc3nnc(C#Cc4cccnc4)cc3N3CCOC4(COC4)[C@@H]3C)C[C@H]2C(=O)O)c2oc3ccccc3c2n1] | 3-ethynylpyridine and (2S,4S)-4-((6- chloro-4-((S)-9- methyl-2,5-dioxa- 8- azaspiro[3.5] nonan-8- yl)pyridazin- 3-yl)oxy)-1-(2- ethylbenzofuro[3, 2-d]pyrimidin-4- yl)pyrrolidine-2- carboxylic acid (Intermediate 33) | LCMS (ESI): mass calcd for C₃₅H₃₃N₇0₆, 647.2; m/z found, 648.6 [M + H]⁺. ¹H NMR (400 MHz, DMSO-d₆) δ 8.81 (d, J = 2.6 Hz, 1H), 8.63 (dd, J = 5.0, 2.1 Hz, 1H), 8.11-7.98 (m, 2H), 7.73-7.60 (m, 2H), 7.53-7.40 (m, 2H), 7.13 (d, J = 7.6 Hz, 1H), 6.16-5.81 (m, 1H), 5.30- 4.95 (m, 1H), 4.65 (p, J = 6.6 Hz, 1H), 4.58-4.47 (m, 2H), 4.37 (d, J = 7.1 Hz, 1H), 4.29 (d, J = 6.7 Hz, 1H), 4.14 (d, J = 6.9 Hz, 1H), 4.08-3.91 (m, 1H), 3.80 (d, J = 12.0 Hz, 1H), 3.67 (qd, J = 11.7, 4.3 Hz, 1H), 3.55-3.45 (m, 1H), 2.99 (ddd, J = 15.5, 9.7, 5.4 Hz, 1H), 2.82 (dq, J = 12.3, 7.5 Hz, 3H), 2.63 (d, J = 14.2 Hz, 1H), 1.31 (dt, J = 10.2, 7.6 Hz, 3H), 1.05-0.85 (m, 2H). |
| | (2S,4S)-1-(2-Ethylbenzofuro[3,2- | | |
| | d]pyrimidin-4-yl)-4-((4-((S)-9-methyl- | | |
| | 2,5-dioxa-8-azaspiro[3.5]nonan-8-yl)- | | |
| | 6-(pyridin-3-ylethynyl)pyridazin-3- | | |
| | yl)oxy)pyrrolidine-2-carboxylic acid | | |
| 224 | 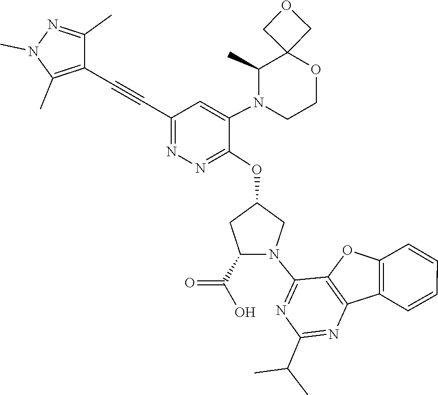 [SMILES: Cc1nn(C)c(C)c1C#Cc1cc(N2CCOC3(COC3)[C@@H]2C)c(O[C@H]2C[C@@H](C(=O)O)N(c3nc(C(C)C)nc4c3oc3ccccc34)C2)nn1] | 4-ethynyl-1,3,5- trimethyl-1H- pyrazole and (2S,4S)-4-((6- chloro-4-((S)-9- methyl-2,5-dioxa- 8- azaspiro[3.5] nonan-8- yl)pyridazin- 3-yl)oxy)-1-(2- isopropylbenzo- furo[3,2- d]pyrimidin-4- yl)pyrrolidine-2- carboxylic acid (Intermediate 30) | LCMS (ESI): mass calcd for C₃₇H₄₀N₈O₆, 692.3; m/z found, 693.3 [M + H]⁺. ¹H NMR (400 MHz, DMSO-d₆) δ 8.08 (d, J = 8.0 Hz, 1H), 7.75-7.69 (m, 1H), 7.68- 7.62 (m, 1H), 7.51-7.42 (m, 1H), 6.98 (s, 1H), 5.89-5.79 (m, 1H), 5.21- 5.10 (m, 1H), 4.70-4.62 (m, 1H), 4.60- 4.50 (m, 3H), 4.42-4.36 (m, 1H), 4.33-4.24 (m, 1H), 4.16-4.11 (m, 1H), 3.84-3.76 (m, 1H), 3.70 (s, 3H), 3.68-3.60 (m, 1H), 3.29-3.23 (m, 2H), 3.00-2.94 (m, 2H), 2.66-2.58 (m, 1H), 2.35 (s, 3H), 2.23 (s, 3H), 1.36- 1.30 (m, 6H), 0.97 (d, J = 6.8 Hz, 3H). |
| | (2S,4S)-1-(2- | | |
| | Isopropylbenzofuro[3,2-d]pyrimidin- | | |
| | 4-yl)-4-((4-((S)-9-methyl-2,5-dioxa-8- | | |
| | azaspiro[3.5]nonan-8-yl)-6-((1,3,5- | | |
| | trimethyl-1 H-pyrazol-4- | | |
| | yl)ethynyl)pyridazin-3- | | |
| | yl)oxy)pyrrolidine-2-carboxylic acid | | |
| 225 | 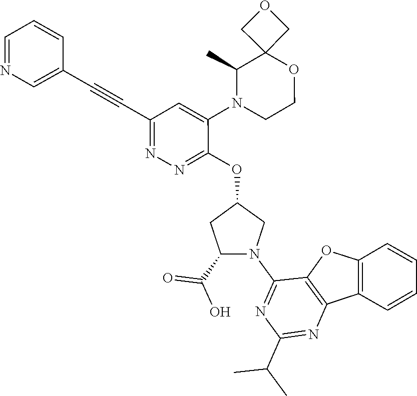 [SMILES: CC(C)c1nc(N2C[C@@H](Oc3nnc(C#Cc4cccnc4)cc3N3CCOC4(COC4)[C@@H]3C)C[C@H]2C(=O)O)c2oc3ccccc3c2n1] | 3-ethynylpyridine and (2S,4S)-4-((6- chloro-4-((S)-9- methyl-2,5-dioxa- 8- azaspiro[3.5] nonan-8- yl)pyridazin- 3-yl)oxy)-1-(2- isopropylbenzo- furo[3,2- d]pyrimidin-4- yl)pyrrolidine-2- carboxylic acid (Intermediate 30) | LCMS (ESI): mass calcd for C₃₆H₃₅N₇O₆, 661.3; m/z found, 662.3 [M + H]⁺. ¹H NMR (400 MHz, DMSO-d₆) δ 8.82 (s, 1H), 8.66-8.63 (m, 1H), 8.08 (d, J = 8.0 Hz, 1H), 8.05-8.01 (m, 1H), 7.74- 7.68 (m, 1H), 7.67-7.62 (m, 1H), 7.52- 7.43 (m, 2H), 7.15 (s, 1H), 5.91-5.82 (m, 1H), 5.19-5.11 (m, 1H), 4.69- 4.63 (m, 1H), 4.61-4.49 (m, 3H), 4.42- 4.37 (m, 1H), 4.34-4.22 (m, 1H), 4.17-4.12 (m, 1H), 3.84-3.78 (m, 1H), 3.72-3.61 (m, 1H), 3.32-3.28 (m, 2H), 2.98-2.94 (m, 2H), 2.65- 2.58 (m, 1H), 1.35-1.31 (m, 6H), 1.00 (d, J = 6.4 Hz, 3H). |
| | (2S,4S)-1-(2-Isopropylbenzofuro[3,2- | | |
| | djpyrimidin-4-yl)-4-((4-((S)-9-methyl- | | |
| | 2,5-dioxa-8-azaspiro[3.5]nonan-8-yl)- | | |
| | 6-(pyridin-3-ylethynyl)pyridazin-3- | | |
| | yl)oxy)pyrrolidine-2-carboxylic acid | | |
| 226 | 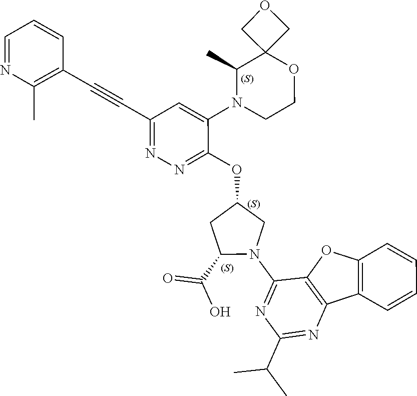 | 3-ethynyl-2- methylpyridine and (2S,4S)-4-((6- chloro-4-((S)-9- methyl-2,5-dioxa- 8-azaspiro[3.5] nonan-8- yl)pyridazin- 3-yl)oxy)-1-(2- isopropylbenzo- furo[3,2- d]pyrimidin-4- yl)pyrrolidine-2- carboxylic acid (Intermediate 30) | LCMS (ESI): mass calcd for C₃₇H₃₇N₇O₆, 675.3; m/z found, 676.3 [M + H]⁺. ¹H NMR (400 MHz, DMSO-d₆) δ 8.53- 8.48 (m, 1H), 8.09 (d, J = 7.6 Hz, 1H), 7.94 (d, J = 8.0 Hz, 1H), 7.75-7.69 (m, 1H), 7.68-7.62 (m, 1H), 7.50-7.43 (m, 1H), 7.34-7.28 (m, 1H), 7.12 (s, 1H), 5.91-5.84 (m, 1H), 5.21-5.12 (m, 1H), 4.71-4.63 (m, 1H), 4.62- 4.49 (m, 3H), 4.41-4.35 (m, 1H), 4.35- 4.26 (m, 1H), 4.16-4.12 (m, 1H), 3.84-3.77 (m, 1H), 3.70-3.61 (m, 1H), 3.30-3.28 (m, 2H), 3.00-2.97 (m, 2H), 2.71 (s, 3H), 2.65-2.59 (m, 1H), 1.35-1.30 (m, 6H), 0.99 (d, J = 6.4 Hz, 3H). |
| | (2S,4S)-1-(2-Isopropylbenzofuro[3,2- | | |
| | d]pyrimidin-4-yl)-4-((4-((S)-9-methyl- | | |
| | 2,5-dioxa-8-azaspiro[3.5]nonan-8-yl)- | | |
| | 6-((2-methylpyridin-3- | | |
| | yl)ethynyl)pyridazin-3- | | |
| | yl)oxy)pyrrolidine-2-carboxylic acid | | |
| 227 | 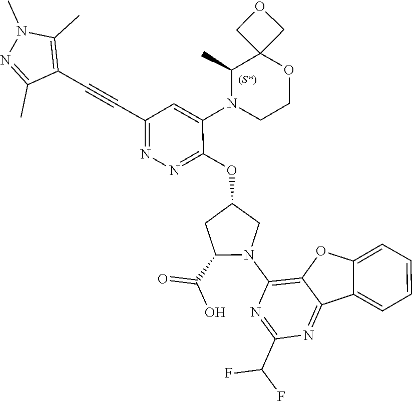 | 4-ethynyl-1,3,5- trimethyl-1H- pyrazole and (2S,4S)-4-((6- chloro-4-((S*)-9- methyl-2,5-dioxa- 8-azaspiro [3.5]nonan- 8-yl)pyridazin-3- yl)oxy)-1-(2- (difluoromethyl) benzofuro [3,2- d]pyrimidin-4- yl)pyrrolidine-2- carboxylic acid (Intermediate 62) | LCMS (ESI): mass calcd for C₃₅H₃₄F₂N₈O₆ 700.3, m/z found, 701.4 [M + H]⁺. ¹H NMR (400 MHz, DMSO-d₆) 8 8.13 (d, J = 7.8 Hz, 1H), 7.83-7.61 (m, 2H), 7.52 (t, J = 7.5 Hz, 1H), 6.96 (s, 1H), 6.77 (t, J = 54.8 Hz, 1H), 5.82 (s, 1H), 5.26 (s, 1H), 4.61 (d, J = 6.8 Hz, 1H), 4.52 (q, J = 6.7 Hz, 3H), 4.37 (d, J = 6.9 Hz, 1H), 4.12 (d, J = 6.9 Hz, 1H), 3.83-3.75 (m, 1H), 3.69 (s, 3H), 3.67- 3.59 (m, 1H), 2.98 (ddd, J = 15.2, 9.9, 5.9 Hz, 1H), 2.69 (d, J = 14.4 Hz, 1H), 2.35 (s, 3H), 2.22 (s, 3H), 2.04 (s, 3H), 0.95 (d, J = 6.5 Hz, 3H). |
| | (2S,4S)-1-(2- | | |
| | (Difluoromethyl)benzofuro[3,2- | | |
| | d]pyrimidin-4-yl)-4-((4-((S*)-9-methyl- | | |
| | 2,5-dioxa-8-azaspiro[3.5]nonan-8-yl)- | | |
| | 6-((1,3,5-trimethyl-1H-pyrazol-4- | | |
| | yl)ethynyl)pyridazin-3- | | |
| | yl)oxy)pyrrolidine-2-carboxylic acid; | | |
| 228 | 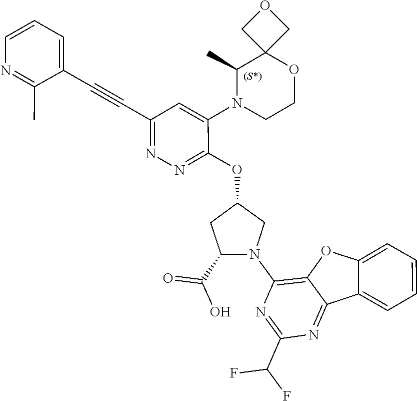 [SMILES: Cc1ncccc1C#Cc1cc(N2CCOC3(COC3)[C@@H]2C)c(O[C@H]2C[C@@H](C(=O)O)N(c3nc(C(F)F)nc4c3oc3ccccc34)C2)nn1] | 3-ethynyl-2- methylpyridine and (2S,4S)-4-((6- chloro-4-((S*)-9- methyl-2,5-dioxa- 8-azaspiro[3.5] nonan-8- yl)pyridazin- 3-yl)oxy)-1-(2- (difluoromethyl)b enzofuro[3,2- d]pyrimidin-4- yl)pyrrolidine-2- carboxylic acid (Intermediate 62) | LCMS (ESI): mass calcd for C₃₅H₃₁F₂N₇O₆ 683.2, m/z found, 684.4 [M + H]⁺. ¹H NMR (400 MHz, DMSO) δ 8.49 (dd, J = 4.9, 1.8 Hz, 1H), 8.13 (d, J = 7.8 Hz, 1H), 7.93 (dd, J = 7.8, 1.8 Hz, 1H), 7.77-7.66 (m, 2H), 7.51 (t, J = 7.4 Hz, 1H), 7.30 (dd, J = 7.8, 4.9 Hz, 1H), 7.10 (s, 1H), 6.76 (t, J = 54.8 Hz, 1H), 5.84 (s, 1H), 5.26 (s, 1H), 4.73-4.43 (m, 4H), 4.37 (d, J = 6.9 Hz, 1H), 4.24 (s, 2H), 4.12 (d, J = 6.9 Hz, 1H), 3.79 (d, J = 11.8 Hz, 1H), 3.64 (td, J = 11.4, 4.4 Hz, 1H), 2.98 (ddd, J = 15.3, 10.0, 5.8 Hz, 1H), 2.70 (s, 3H), 2.03 (s, 2H), 0.98 (d, J = 6.6 Hz, 3H). |
| | (2S,4S)-1-(2- | | |
| | (Difluoromethyl)benzofuro[3,2- | | |
| | d]pyrimidin-4-yl)-4-((4-((S*)-9-methyl- | | |
| | 2,5-dioxa-8-azaspiro[3.5]nonan-8-yl)- | | |
| | 6-((2-methylpyridin-3- | | |
| | yl)ethynyl)pyridazin-3- | | |
| | yl)oxy)pyrrolidine-2-carboxylic acid | | |
| 229 | 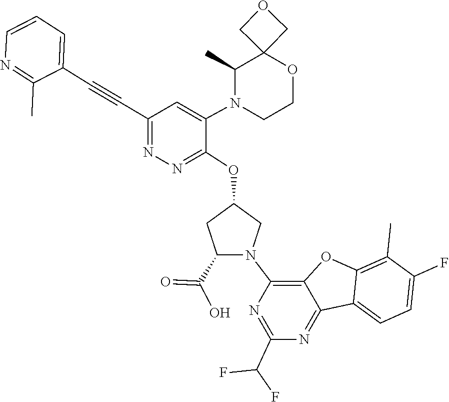 [SMILES: Cc1ncccc1C#Cc1cc(N2CCOC3(COC3)[C@@H]2C)c(O[C@H]2C[C@@H](C(=O)O)N(c3nc(C(F)F)nc4c3oc3c(C)c(F)ccc34)C2)nn1] | 3-ethynyl-2- methylpyridine and (2S,4S)-4-((6- chloro-4-((S)-9- methyl-2,5-dioxa- 8-azaspiro[3.5] nonan-8- yl)pyridazin-3- yl)oxy)-1-(2- (difluoromethyl)-7- fluoro-6- methylbenzofuro [3,2-d]pyrimidin-4- carboxylic acid (Intermediate 63). | LCMS (ESI): mass calcd for C₃₆H₃₂F₂N₇O₆ 715.2, m/z found, 716.6 [M + H]⁺. ¹H NMR (400 MHz, DMSO) δ 8.46 (dd, J = 4.9, 1.7 Hz, 1H), 7.98- 7.86 (m, 2H), 7.41-7.14 (m, 2H), 7.07 (s, 1H), 6.73 (t, J = 54.7 Hz, 1H), 5.81 (s, 1H), 5.31 (s, 1H), 4.80-4.55 (m, 1H), 4.55-4.37 (m, 3H), 4.31 (d, J = 6.9 Hz, 1H), 4.09 (d, J = 6.9 Hz, 1H), 3.76 (dd, J = 11.8, 2.8 Hz, 1H), 3.61 (dt, J = 11.6, 7.4 Hz, 1H), 3.23 (s, 1H), 3.01 (s, 1H), 2.67 (s, 3H), 2.47 (s, 3H), 2.43 (s, 3H), 0.94 (d, J = 6.5 Hz, 3H). |
| | (2S,4S)-1-(2-(Difluoromethyl)-7- | | |
| | fluoro-6-methylbenzofyro[3,2- | | |
| | d]pyrimidin-4-yl)-4-((4-((S)-9-methyl | | |
| | 2,5-dioxa-8-azaspiro[3.5]nonan-8-yl)- | | |
| | 6-((2-methylpyridin-3- | | |
| | yl)ethynyl)pyridazin-3- | | |
| | yl)oxy)pyrrolidine-2-carboxylic acid | | |

### Biological Data

### 1A. Potency Data for Example Compounds

THP1, an immortalized human monocyte cell line, was engineered to contain two inducible reporters, namely SEAP and Luciferase (Lucia) under promotors responsive to NF-kB and IRF, respectively, to create the THP1-Dual (Invivogen) cell line. In Assay No. 1, stimulation of the cells was accomplished with double-stranded DNA, such as VACV-70/LyoVec (Invivogen), which activates the cGAS pathway to initiate downstream signaling and expression of the reporter genes. In Assay No. 2, stimulation of the cells was accomplished by induction of mtDNA release with ABT-737 and Q-VD-OPH (Sigma), which activates the cGAS pathway to initiate downstream signaling and expression of the reporter genes. Cells were maintained in suspension in culture medium consisting of the following: RPMI 1640 with GlutaMAX, 10% Heat Inactivated Fetal Bovine Serum, 100U/mL Pen/Strep, 100 ug/mL Normocin, 10 μg/mL Blasticidin, and 100 μg/mL Zeocin. Cells were treated with compounds diluted to a final concentration of 0.2% DMSO for 1 hour prior to addition of DNA. Cells were stimulated with VACV-70/Lyovec to a final concentration of 5 ug/mL or ABT-737/Q-VD-OPH to a final concentration of 10 uM for 18-21 hours in 37° C./5% CO₂ incubator. Supernatants were collected and luciferase detection was performed using Quanti Luc reagents (Invivogen). Luciferase signal was measured using a Pherastar instrument (BMG Labtech) using MARS software.

TABLE 22
Potency Data for Example Compounds

| | Assay No. 1 | Assay No. 2 |
|---|---|---|
| Ex # | IC₅₀ (uM) | IC₅₀ (uM) |
| 1 | 0.0009 | 0.0024 |
| 2 | 0.0022 | 0.0044 |
| 3 | 0.0119 | 0.0256 |
| 4 | 0.0009 | 0.0017 |
| 5 | 0.0173 | 0.0433 |
| 6 | 0.0131 | 0.0412 |
| 7 | 0.0076 | 0.0242 |
| 8 | 0.0035 | 0.0132 |
| 9 | 0.0117 | 0.0243 |
| 11 | 0.0122 | 0.0131 |
| 12 | 0.0326 | 0.0841 |
| 13 | 0.0220 | 0.0437 |
| 14 | 0.0102 | 0.0140 |
| 15 | 0.0378 | 0.1161 |
| 16 | 0.0049 | 0.0112 |
| 17 | 0.0078 | 0.0208 |
| 18 | 0.0532 | 0.1188 |
| 19 | 0.0241 | 0.0435 |
| 20 | 0.0031 | 0.0074 |
| 21 | 0.0018 | 0.0062 |
| 22 | 0.0008 | 0.0006 |
| 23 | 0.1923 | 0.3980 |
| 24 | 0.0028 | 0.0076 |
| 25 | 0.0049 | 0.0226 |
| 26 | 0.0008 | 0.0031 |
| 27 | 0.0007 | 0.0008 |
| 28 | 0.0008 | 0.0021 |
| 29 | 0.1935 | 0.3986 |
| 30 | 0.0027 | 0.0029 |
| 31 | 0.0077 | 0.0291 |
| 32 | 0.0404 | 0.1860 |
| 33 | 0.0005 | 0.0009 |
| 34 | 0.0327 | 0.0895 |
| 35 | 0.0057 | 0.0207 |
| 36 | 0.0007 | 0.0006 |
| 37 | 0.1089 | 0.6981 |
| 38 | 0.0008 | 0.0016 |
| 39 | 0.2700 | — |
| 40 | 0.0026 | 0.0083 |
| 41 | 0.4631 | 0.2630 |
| 42 | 0.0007 | 0.0021 |
| 43 | 0.0023 | 0.0061 |
| 44 | 0.0022 | 0.0021 |
| 45 | 0.0070 | 0.0124 |
| 46 | 0.0008 | 0.0011 |
| 47 | 0.0017 | 0.0037 |
| 48 | 0.0022 | 0.0040 |
| 49 | 0.0015 | 0.0028 |
| 50 | 0.0196 | 0.0502 |
| 51 | 0.0003 | 0.0008 |
| 52 | 0.0030 | 0.0036 |
| 53 | 0.0011 | 0.0018 |
| 54 | 0.0011 | 0.0023 |
| 55 | 0.0023 | 0.0030 |
| 56 | 0.0028 | 0.0084 |
| 57 | 0.0085 | 0.0260 |
| 58 | 0.0012 | 0.0023 |
| 59 | 0.0018 | 0.0076 |
| 60 | 0.0008 | 0.0010 |
| 61 | 0.0029 | 0.0049 |
| 62 | 0.0026 | 0.0094 |
| 63 | 0.0046 | 0.0172 |
| 64 | 0.0012 | 0.0025 |
| 65 | 0.0216 | 0.0626 |
| 66 | 0.0496 | 0.1708 |
| 67 | 0.0012 | 0.0025 |
| 68 | 0.0130 | 0.0302 |
| 69 | 0.0368 | 0.1196 |
| 70 | 0.0016 | 0.0010 |
| 71 | 0.0012 | 0.0023 |
| 72 | 0.0033 | — |
| 73 | 0.0037 | 0.0098 |
| 74 | 0.0009 | 0.0009 |
| 75 | 0.0050 | 0.0095 |
| 76 | 0.0074 | 0.0173 |
| 77 | 0.0012 | 0.0012 |
| 78 | 0.0124 | 0.0438 |
| 79 | 0.0017 | 0.0030 |
| 80 | 0.0028 | 0.0103 |
| 81 | 0.0018 | 0.0042 |
| 82 | 0.0035 | 0.0082 |
| 83 | 0.0016 | 0.0021 |
| 84 | 0.0012 | 0.0022 |
| 85 | 0.0098 | 0.0240 |
| 86 | 0.0059 | 0.0196 |
| 87 | 0.0039 | 0.0091 |
| 88 | 0.0038 | 0.0138 |
| 89 | 0.0008 | 0.0007 |
| 90 | 0.0026 | 0.0013 |
| 91 | 0.0030 | 0.0015 |
| 92 | 0.0012 | 0.0007 |
| 93 | 0.0017 | 0.0009 |
| 94 | 0.0047 | 0.0034 |
| 95 | 0.0024 | 0.0011 |
| 96 | 0.0014 | 0.0007 |
| 97 | 0.0010 | 0.0004 |
| 98 | 0.0009 | 0.0005 |
| 99 | 0.0029 | 0.0009 |
| 100 | 0.0042 | 0.0024 |
| 101 | 0.0133 | 0.0040 |
| 102 | 0.0027 | 0.0019 |
| 103 | 0.0074 | 0.0039 |
| 104 | 0.0141 | 0.0078 |
| 105 | 0.0067 | 0.0048 |
| 106 | 0.0148 | 0.0089 |
| 107 | 0.0034 | 0.0018 |
| 108 | 0.0024 | 0.0011 |
| 109 | 0.0034 | 0.0020 |
| 110 | 0.0094 | 0.0104 |
| 111 | 0.0132 | 0.0039 |
| 112 | 0.0453 | 0.0132 |
| 113 | 0.0112 | 0.0045 |
| 114 | 0.0041 | 0.0028 |
| 115 | 0.0411 | 0.0250 |
| 116 | 0.0075 | 0.0057 |
| 117 | 0.0260 | 0.0154 |
| 118 | 0.0040 | 0.0022 |
| 119 | 0.0066 | 0.0020 |
| 120 | 0.0042 | 0.0040 |
| 121 | 0.0072 | 0.0046 |
| 122 | 0.0031 | 0.0015 |
| 123 | 0.0100 | 0.0083 |
| 124 | 0.0036 | 0.0024 |
| 125 | 0.0473 | 0.0200 |
| 126 | 0.0021 | 0.0020 |
| 127 | 0.0016 | 0.0021 |
| 128 | 0.0017 | 0.0056 |
| 129 | — | 0.0018 |
| 130 | 0.0024 | 0.0015 |
| 131 | 0.0005 | 0.0023 |
| 132 | 0.0056 | 0.0296 |
| 133 | 0.0038 | 0.0039 |
| 134 | 0.0004 | 0.0005 |
| 135 | 0.0047 | 0.0115 |
| 136 | 0.0004 | — |
| 137 | 0.0011 | — |
| 138 | 0.0036 | 0.0123 |
| 139 | 0.0011 | 0.0012 |
| 140 | 0.0021 | 0.0023 |
| 141 | 0.0114 | 0.0089 |
| 142 | 0.0124 | 0.0185 |
| 143 | 0.0089 | 0.0080 |
| 144 | 0.0210 | 0.0202 |
| 145 | 0.0022 | 0.0064 |
| 146 | 0.0007 | 0.0009 |
| 147 | 0.0005 | — |
| 148 | 0.0008 | 0.0031 |
| 149 | 0.0050 | 0.0023 |
| 150 | 0.0027 | 0.0029 |
| 151 | 0.0403 | 0.0400 |
| 152 | 0.0014 | 0.0010 |
| 153 | 0.0223 | 0.0076 |
| 154 | 0.0143 | 0.0027 |
| 155 | 0.0270 | 0.0163 |
| 156 | 0.0014 | 0.0009 |
| 157 | 0.0507 | 0.1250 |
| 158 | 0.0091 | 0.0064 |
| 159 | 0.0014 | 0.0014 |
| 160 | 0.0028 | 0.0029 |
| 161 | 0.0117 | 0.0184 |
| 162 | 0.0875 | 0.1242 |
| 163 | 0.0008 | 0.0014 |
| 164 | 0.0009 | 0.0006 |
| 165 | 0.0047 | 0.0139 |
| 166 | 0.0014 | 0.0012 |
| 167 | 0.0019 | 0.0011 |
| 168 | 0.0011 | 0.0009 |
| 169 | 0.0030 | 0.0026 |
| 170 | 0.0020 | 0.0010 |
| 171 | 0.0016 | 0.0011 |
| 172 | 0.0097 | 0.0182 |
| 173 | 0.0456 | 0.0316 |
| 174 | 0.0017 | 0.0021 |
| 175 | 0.0064 | 0.0125 |
| 176 | 0.0039 | 0.0079 |
| 177 | 0.0435 | 0.0513 |
| 178 | 0.0025 | 0.0077 |
| 179 | 0.0011 | 0.0028 |
| 180 | 0.0005 | 0.0024 |
| 181 | 0.0016 | 0.0013 |
| 182 | 0.0013 | 0.0016 |
| 183 | 0.0007 | 0.0020 |
| 184 | 0.0013 | 0.0005 |
| 185 | 0.0013 | 0.0009 |
| 186 | 0.0038 | 0.0036 |
| 187 | 0.0024 | 0.0082 |
| 188 | 0.0014 | 0.0013 |
| 189 | 0.0007 | 0.0004 |
| 190 | 0.0021 | 0.0013 |
| 191 | 0.0018 | 0.0020 |
| 192 | 0.0094 | 0.0255 |
| 193 | 0.0384 | 0.0958 |
| 194 | 0.0070 | 0.0379 |
| 195 | 0.0018 | 0.0009 |
| 196 | 0.0017 | 0.0012 |
| 197 | 0.0021 | 0.0021 |
| 198 | 0.0033 | 0.0019 |
| 199 | 0.0062 | 0.0032 |
| 200 | 0.0262 | 0.0147 |
| 201 | 0.0021 | 0.0014 |
| 202 | 0.0188 | 0.0095 |
| 203 | 0.0082 | 0.0039 |
| 204 | 0.0086 | 0.0049 |
| 205 | 0.0145 | 0.0104 |
| 206 | 0.0055 | 0.0037 |
| 207 | 0.0040 | 0.0019 |
| 208 | 0.0013 | 0.0037 |
| 209 | 0.0014 | 0.0016 |
| 210 | 0.0033 | 0.0031 |
| 211 | 0.0018 | 0.0023 |
| 212 | 0.0024 | 0.0028 |
| 213 | 0.0019 | 0.0019 |
| 214 | 0.0016 | 0.0097 |
| 215 | 0.0023 | 0.0049 |
| 216 | 0.0024 | 0.0023 |
| 217 | 0.0029 | 0.0038 |
| 218 | 0.0023 | 0.0022 |
| 219 | 0.0011 | 0.0010 |
| 220 | 0.0043 | 0.0055 |
| 221 | 0.0306 | 0.0443 |
| 222 | 0.0141 | 0.0188 |
| 223 | 0.0031 | 0.0025 |
| 224 | 0.0015 | 0.0009 |
| 225 | 0.0071 | 0.0068 |
| 226 | 0.0016 | 0.0009 |
| 227 | 0.0023 | 0.0013 |
| 228 | 0.0008 | 0.0007 |
| 229 | 0.0009 | 0.0005 |

“—” means data not collected

### 1B. Discussion of Potency Data and Modeling

As shown in Table 22, compounds of Formula (I) and Formula (II) are potent inhibitors of cGAS with IC₅₀ values ranging from 0.0003 μM to 0.4631 μM as measured in Assay No. 1 and values ranging from 0.0004 μM to 0.6981 μM as measured in Assay No. 2. These assays measure cGAS-inhibition in THP1-Dual cells stimulated with either double-stranded DNA (Assay No. 1) or mtDNA release with ABT-737 and Q-VD-OPH (Assay No. 2).

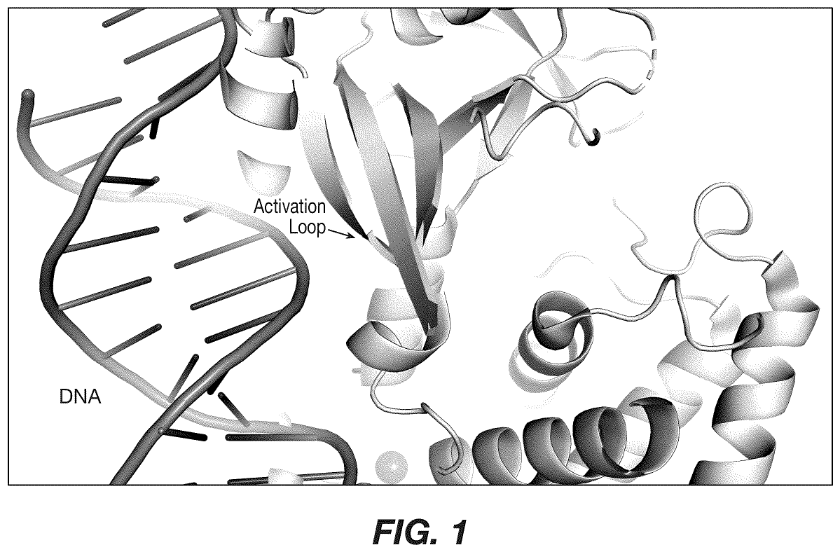

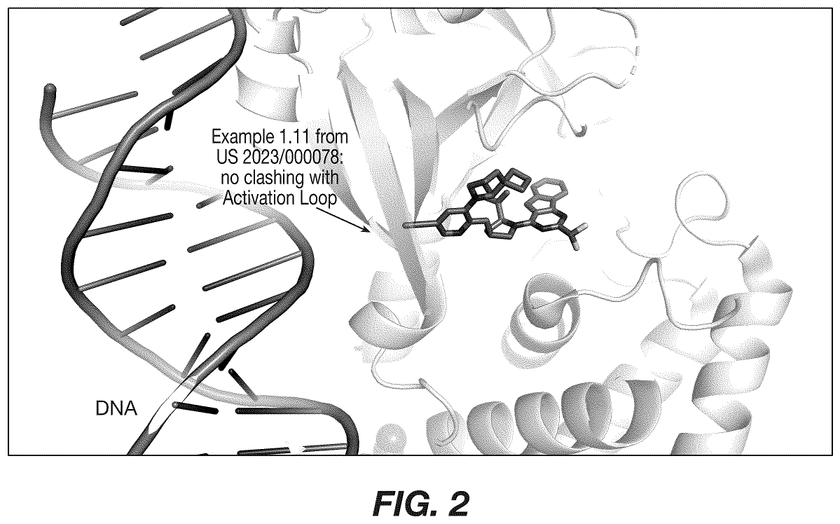

The high potency of the compounds described herein is surprising in view of the sterically hindered binding pocket in the target. This steric hinderance is illustrated by FIG. 1, which shows the cGAS-DNA bound structure from W. Xie, et al., “Human cGAS catalytic domain has an additional DNA-binding interface that enhances enzymatic activity and liquid-phase condensation,” Proc. Natl. Acad. Sci. U.S.A. 116 (24) 11946-11955, https//doi.org/10.1073/pnas.1905013116 (2019). As can be seen, the activation loop in the cGAS-DNA bound structure (amino acids 210-220) appears to restrict the space available for a small molecule compound to interact with cGAS-DNA bound structure. This view is supported by modeling Example 1.11 from US 2023/000078 with the cGAS-DNA bound structure, which is shown in FIG. 2. There, Ex. 1.11 (structure reproduced below) is in an energetically minimized structure in water, then inserted in cGAS receptor using substructural overlay with a literature structure from US 2022/0073532 (e.g., FIG. 4 in US 2022/0073532). As shown in FIG. 2, the terminal alkynyl group attached to the pyridine ring appears to abut the activation loop without causing structural alteration of the target.

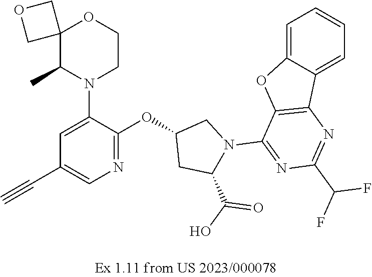 [SMILES: C#Cc1cnc(O[C@H]2C[C@@H](C(=O)O)N(c3nc(C(F)F)nc4c3oc3ccccc34)C2)c(N2CCOC3(COC3)[C@@H]2C)c1]

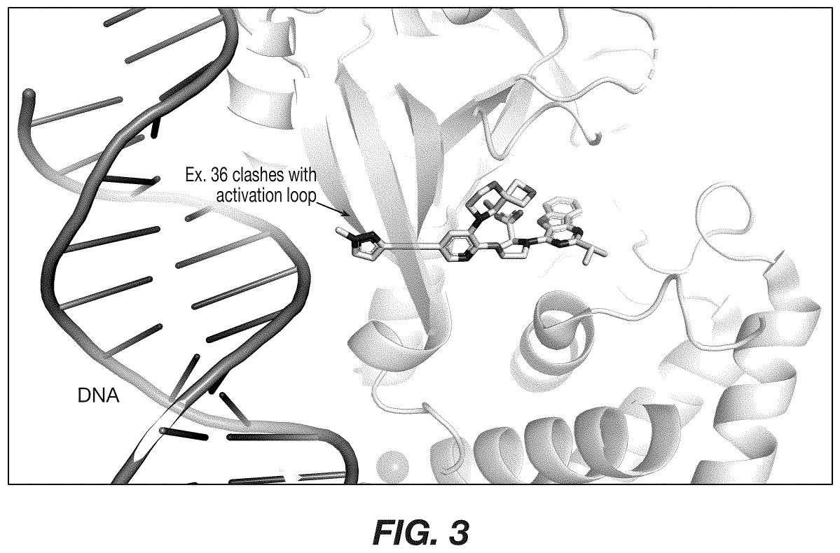

On the other hand, compounds of Formula (II) as described herein, would have been expected to be too large to bind within the binding pocket in the cGAS-DNA bound structure—let alone exhibit the high potencies summarized in Table 22. To illustrate this point, Example 36 was modeled with the cGAS-DNA bound structure as shown in FIG. 3. There, Example 36 is in an energetically minimized conformation in water, then inserted in cGAS receptor using substructural overlay with a literature structure from US 2022/0073532 (e.g., FIG. 4 from US 2022/0073532). As can be seen in FIG. 3, there is clear steric clashing between the higher order heteroaryl alkynyl group of Example 36 and the activation loop in the cGAS-DNA bound structure. Accordingly, compounds of Formula (II), such as Example 36, would appear too large to bind within the sterically constrained binding pocket of the cGAS-DNA bound structure (see below).

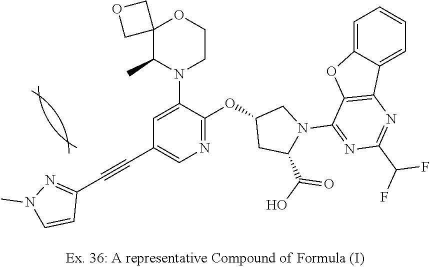 [SMILES: C[C@@H]1N(c2cc(C#Cc3ccn(C)n3)cnc2O[C@H]2C[C@@H](C(=O)O)N(c3nc(C(F)F)nc4c3oc3ccccc34)C2)CCOC12COC2]

The larger R² groups shown in the US 2023/000078 examples do not change that compounds of formula (II) have unexpectedly high potency in view of the sterically hindered binding pocket in the cGAS target. While US 2023/000078 shows that the lower order alkynyl R² group can be replaced with a larger group such as the heteroaryl group in Example 4.01 (see entry 4 in Table 23), as a whole, it shows that compounds with smaller R² groups (e.g., R² is chloro, fluoro, cyclopropyl, C₁₋₃alkyl, C₂₋₅alkynyl, —S-methyl, or —CN) have increased potency over larger R² groups. This trend is apparent from analysis of the THP1 cell assay data of other examples in US 2023/0000878 as compared to representative example 4.01. For instance, examples such as 1.09, 1.11, 1.12, 1.13, 1.34, 2.03, 2.04, 6.01, 6.02, 9.01, 3.15, 3.19, 3.22, 3.24, 3.26, 3.27, 3.28, 3.29, 4.03, 5.01 differ from Ex. 4.01 at the R² position and have either methyl, monofluoromethyl, difluoromethyl or trifluoromethyl at R¹. Exemplified compounds that have smaller R² groups (F, Cl, -ethynyl, 1-propynyl, and —SMe as in examples 1.09, 1.11, 1.12, 1.13, 1.34, 2.03, 2.04, 6.01, 6.02, 9.01) all exhibit cellular potency of less than 40 nM. On the other hand, exemplified compounds with aryl R² groups, have cellular potency that span a larger activity range of up to 712 nM, with several of such aryl R² examples having weaker IC₅₀ values of >40 nM (e.g., examples 3.15, 3.19, 3.22, 3.24, 3.26, 3.27, 3.28, 3.29, 4.03, 5.01). Moreover, Ex. 3.29 in US 2023/0000878 possesses one of the larger R² groups in the examples (R² is pyridine substituted with —O-tetrahydropyran) and it is one of the weakest potency compounds (712 nM).

### 2A. Comparative Data Analysis

Table 23 describes potency data and pharmacological properties for Example Compound Nos. 44, 96, 98, 99, 107, and 108 in the instant disclosure and representative compounds from US 2023/0000878. The data for both groups of compounds was collected using identical procedures, and therefore, can be used for comparison purposes.

### Permeability Assay

The in vitro permeability of test compounds is determined using Madin-Darby canine kidney (MDCK) cells to provide a measure of intestinal absorption. MDCK cells are seeded onto Millipore Multiscreen Transwell plates and cultured for 4 days. To initiate the permeability studies test compound is added to either the apical or basolateral side of a confluent MDCK monolayer and permeability in the apical to basolateral (A-B) and basolateral to apical (B-A) direction is measured by monitoring the appearance of the test compound on the opposite side of the membrane using LCMS/MS. The permeability coefficient for each test compound (Papp) is derived from the rate of permeation across the cell monolayer.

### Stability Assays

The in vitro stability of test compounds is determined in hepatocytes isolated from cynomolgus monkeys to provide a measure of metabolic stability. Cryopreserved hepatocytes are thawed and incubated with test compound (typically 1 μM) for up to 4 hours. The reaction is terminated by addition of organic solvent and samples are centrifuged prior to LC-MS/MS analysis. The in vitro intrinsic clearance (Clint) is calculated using the slope of the log-linear regression from the percentage parent compound remaining versus time relationship.

### Bioavailability Assay

The objective of this study is to determine the pharmacokinetic (PK) profile and oral bioavailability (F) of the test compound following intravenous (0.5 mg/kg) and oral (2.5 mg/kg) administrations to male Cynomolgus monkeys. Test compounds for both IV and oral administration are formulated in 20% HP-beta-cyclodextrin at 1.25 mg/mL. All study groups consist of three naïve male Cynomolgus monkeys with a weight ranging from 2 to 5 kg. Animals are fasted overnight prior to dose administration and up to four hours after dosing. Serial venous blood samples (approximately 0.5 mL) are taken at time points up to 24 hours after dosing from each animal. Blood samples are transferred into blood collection tubes containing EDTA as the anti-coagulant and tubes are immediately placed on wet ice until centrifugation for plasma. After centrifugation, plasma is separated and 0.5M ammonium formate buffer is added and tubes are stored at approximately −20° C. LC-MS/MS analysis is used to determine the concentration of test compounds in plasma and non-compartmental analysis is performed on the plasma concentration-time data. Oral bioavailability is calculated as a percentage by comparing the areas under the plasma concentration-time curve after IV and oral administration. Table 23. Results of In vitro and In vivo Comparative Analysis Compound Potency Data Permeability Cyno hepatocyte Cyno bio-Assay No. 2) data (Papp) Stability availability

TABLE 23
Results of In vitro and In vivo Comparative Analysis

| | | Potency Data | Permeability | Cyno hepatocyte | Cyno bio- |
|---|---|---|---|---|---|
| | Compound | (Assay No. 2) | data (Papp) | Stability | availability |
| 1 | 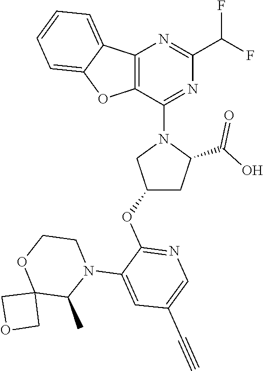 [SMILES: C#Cc1cnc(O[C@H]2C[C@@H](C(=O)O)N(c3nc(C(F)F)nc4c3oc3ccccc34)C2)c(N2CCOC3(COC3)[C@@H]2C)c1] | 1.27 nM | 1.91 × 10⁻⁶ cm/s | 21.5 μL/min/ million cells | 6% |
| | Example No. 1.11 | | | | |
| | US 2023/0000878 | | | | |
| 2 | 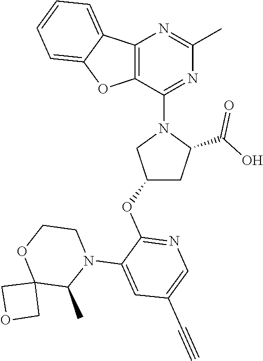 [SMILES: C#Cc1cnc(O[C@H]2C[C@@H](C(=O)O)N(c3nc(C)nc4c3oc3ccccc34)C2)c(N2CCOC3(COC3)[C@@H]2C)c1] | 1.68 nM | 3.33 × 10⁻⁶ cm/s | 16.6 μL/min/ million cells | — |
| | Example No. 2.03 | | | | |
| | US 2023/0000878 | | | | |
| 3 | 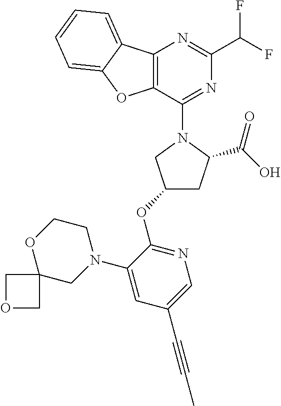 [SMILES: CC#Cc1cnc(O[C@H]2C[C@@H](C(=O)O)N(c3nc(C(F)F)nc4c3oc3ccccc34)C2)c(N2CCOC3(COC3)C2)c1] | 0.653 nM | 2.5 × 10⁻⁶ cm/s | 10.6 μL/min/ million cells | — |
| | Example No. 6.02 | | | | |
| | US 2023/0000878 | | | | |
| 4 | 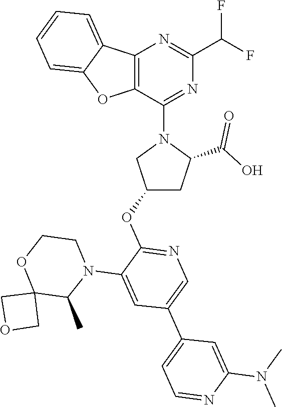 [SMILES: C[C@@H]1N(c2cc(-c3ccnc(N(C)C)c3)cnc2O[C@H]2C[C@@H](C(=O)O)N(c3nc(C(F)F)nc4c3oc3ccccc34)C2)CCOC12COC2] | 1.2 nM | 2.17 × 10⁻⁶ cm/s | 27.9 μ/min/ million cells | 1% |
| | Example No. 4.01 | | | | |
| | US 2023/0000878 | | | | |
| 5 | 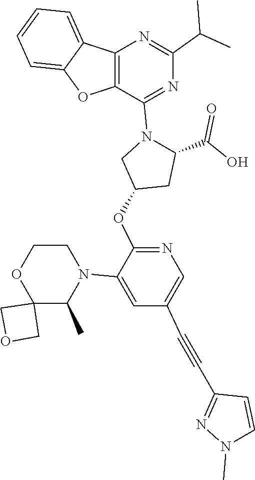 [SMILES: CC(C)c1nc(N2C[C@@H](Oc3ncc(C#Cc4ccn(C)n4)cc3N3CCOC4(COC4)[C@@H]3C)C[C@H]2C(=O)O)c2oc3ccccc3c2n1] | 2.1 nM | 5.51 × 10⁻⁶ cm/s | 5.27 μL/min/ million cells | 34% |
| | Example 44 | | | | |
| 6 | 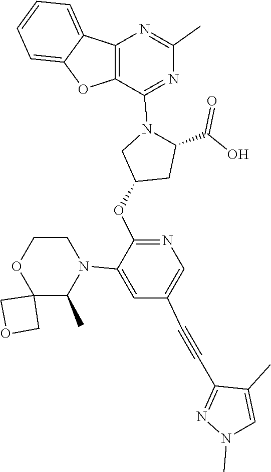 [SMILES: Cc1nc(N2C[C@@H](Oc3ncc(C#Cc4nn(C)cc4C)cc3N3CCOC4(COC4)[C@@H]3C)C[C@H]2C(=O)O)c2oc3ccccc3c2n1] | 0.71 nM | 3.33 × 10⁻⁶ cm/s | 3.65 μL/min/ million cells | — |
| | Example 96 | | | | |
| 7 | 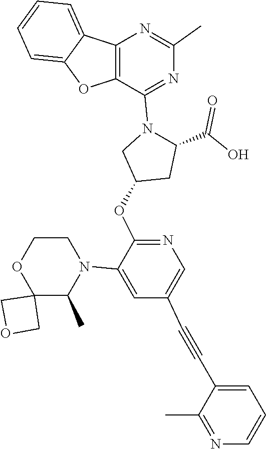 [SMILES: Cc1nc(N2C[C@@H](Oc3ncc(C#Cc4cccnc4C)cc3N3CCOC4(COC4)[C@@H]3C)C[C@H]2C(=O)O)c2oc3ccccc3c2n1] | 0.52 nM | 2.9 × 10⁻⁶ cm/s | 16.36 μL/min/ million cells | — |
| | Example 98 | | | | |
| 8 | 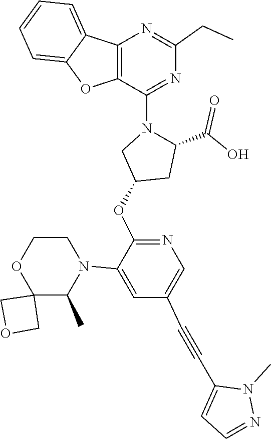 [SMILES: CCc1nc(N2C[C@@H](Oc3ncc(C#Cc4ccnn4C)cc3N3CCOC4(COC4)[C@@H]3C)C[C@H]2C(=O)O)c2oc3ccccc3c2n1] | 0.94 nM | 6.54 × 10⁻⁶ cm/s | 7.92 μL/min/ million cells | — |
| | Example 99 | | | | |
| 9 | 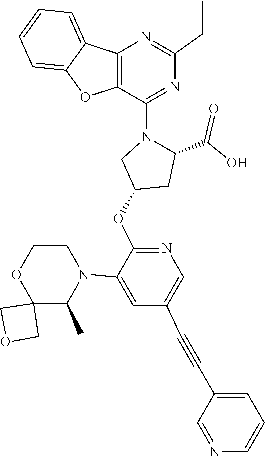 [SMILES: CCc1nc(N2C[C@@H](Oc3ncc(C#Cc4cccnc4)cc3N3CCOC4(COC4)[C@@H]3C)C[C@H]2C(=O)O)c2oc3ccccc3c2n1] | 1.8 nM | 5.59 × 10⁻⁶ cm/s | 8.15 μL/min/ million cells | 21% |
| | Example 107 | | | | |
| 10 | 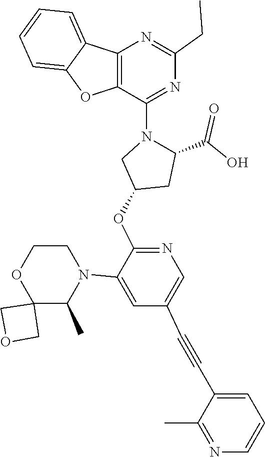 [SMILES: CCc1nc(N2C[C@@H](Oc3ncc(C#Cc4cccnc4C)cc3N3CCOC4(COC4)[C@@H]3C)C[C@H]2C(=O)O)c2oc3ccccc3c2n1] | 1.1 nM | 5.64 × 10⁻⁶ cm/s (average of 8.17 × 10⁻⁶ cm/s and 3.11 × 10⁻⁶ cm/s) | 14.96 μL/min/ million cells | 21% |
| | Example 108 | | | | |

″—″ means data not collected

2B. Discussion of Example Compounds and Representative Compounds from US 2023/0000878

Compounds from US 2023/0000878: US 2023/0000878 describes pyridine derivatives with N-linked cyclic substitutes as cGAS inhibitors (see e.g., Formula (I) below and Table 23 herein, entries 1-4). US 2023/0000878 further states that the R² group, which is attached to the pyridine ring, can be a C₂₋₅alkynyl group in addition to other small groups (e.g., hydrogen, halogen, cyclopropyl, C₁₋₃alkyl, —S-methyl, and CN). US 2023/0000878 at page 2, paragraph [0023]. The “Terms and Definitions” section further states that the term “C₂₋₅alkynyl” means branched and unbranched alkynyl groups with 2 to 5 carbons provided they have at least one triple bond. Id. at page 22, paragraph [0150]. The example section shows that R² can be a terminal alkyne (—C≡C—H) or a methyl alkyne (—C≡C-Me), which is illustrated by representative examples Nos. 1.11, 2.03, and 6.02 (see entries 1-3 of Table 23). No other alkynyl R² groups are exemplified in US 2023/0000878.

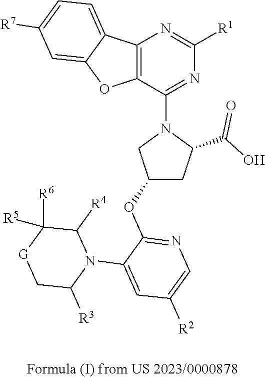

Alternatively, US 2023/0000878 states that R² can be a cyclic group selected from the group consisting of a phenyl and a five- to six-membered heteroaryl comprising 1, 2, 3, or 4 heteroatoms selected from N, S and O. US 2023/0000878 at paragraph [0024]. This cyclic group is substituted by one or two R¹⁰ groups, which are further described in paragraph [0089] of US 2023/0000878. A representative example (Ex. 4.01) where R² is a heteroaryl group is shown in entry 4 of Table 23.

Compounds of Formula II: The instant compounds are structurally differentiated from those in US 2023/0000878 at least because they have a higher order alkynyl group attached to the pyridine ring. Despite the increased sterics of this functionality, the compounds of Formula (II) are potent inhibitors of cGAS with IC₅₀ values ranging from 0.0003 μM to 0.4631 μM (see, e.g., Assay No. 1 in Table 22). For all the reasons described above, this potency was unexpected in view of the sterically constrained cGAS binding pocket (see FIGS. 1-3).

Comparison Data: Moreover, certain compounds of Formula (IIB) maintain their high potency while also exhibiting increased permeability, hepatocyte stability, and/or bioavailability as compared to representative example compounds from US 2023/0000878 (see Table 23). Regarding permeability, Examples 1.11, 2.03, 4.01 and 6.02 from US 2023/0000878 exhibit permeability values of 1.91×10⁻⁶ cm/s, 3.33×10⁻⁶ cm/s, 2.17×10⁻⁶ cm/s and 2.5×10⁻⁶ cm/s, respectively. See entries 1-4 in Table 23. The representative Examples 44, 96, 98, 99, 107, and 108 from the instant disclosure exhibit generally higher permeability values of 5.51×10⁻⁶ cm/s, 3.33×10⁻⁶ cm/s, 2.9×10⁻⁶ cm/s, 6.54×10⁻⁶ cm/s, 5.59×10⁻⁶ cm/s, and 5.64×10⁻⁶ cm/s, respectively. See Table 23, entries 5-10.

Certain compounds of Formula (IIB) have increased stability as compared to the representative compounds from US 2023/0000878. For example, as shown in entries 1-4 of Table 23, representative examples 1.11, 2.03, 4.01 and 6.02 from US 2023/0000878 exhibit stability values of 21.5 μL/min/million cells, 16.6 μL/min/million cells, 27.9 μL/min/million cells, and 10.6 μL/min/million cells, respectively. Examples 44, 96, 99, and 107 from the instant disclosure, however, have increased stability values of 5.27 μL/min/million cells, 3.65 μL/min/million cells, 7.92 μL/min/million cells, and 8.15 μL/min/million cells, respectively. See Table 23, entries 5-6 and 8-9.

Additionally, representative compounds Formula (IIB) have increased bioavailability as compared to the representative compounds 1.11 and 4.01 from US 2023/0000878. Those compounds, for example, have bioavailability values of 6% and 1%, respectively. See entries 1 and 4 of Table 23. In contrast, Examples 44, 107, and 108 from the instant disclosures have increased bioavailability values ranging from 21% to 34%. See entries 5 and 9-10 of Table 23.

In sum, it has been surprisingly found that compounds described herein (e.g., compounds of Formula (II)) are potent inhibitors of cGAS with IC₅₀ values ranging from 0.0003 μM to 0.4631 μM with regards to inhibiting the production of IP-10 in cells stimulated with double-stranded DNA. See e.g., Assay No. 1 described in Table 22. Moreover, certain compounds of Formula (IIB) maintain their high potency while also exhibiting increased permeability, hepatocyte stability, and/or bioavailability as compared to representative examples from US 2023/0000878 (see, e.g., Table 23).

What is claimed is:

1. A compound of Formula (II), or a pharmaceutically acceptable salt thereof, 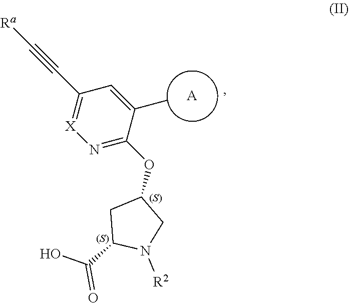
wherein:
\textcircled{A} is 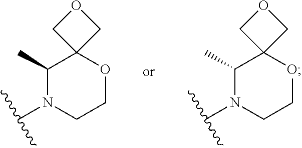 [SMILES: *N1CCOC2(COC2)[C@@H]1C ; *N1CCOC2(COC2)[C@H]1C]
X is CH or N:
R^{a} is selected from the group consisting of:
(viii) —C₁₋₆alkyl substituted with one or two members each independently selected from the group consisting of: —OH and —OC₁₋₆alkyl; or
(ix) —C₃₋₆cycloalkyl or -L¹-C₃₋₆cycloalkyl, wherein the cycloalkyl is monocyclic or fused bicyclic, and unsubstituted or substituted with one or two members each independently selected from the group consisting of: —F, —OH, —OC₁₋₆alkyl, and 1H-pyrazole; wherein -L¹- is —C₁₋₃alkyl; or
(x) phenyl or -L²-phenyl, wherein the phenyl is unsubstituted or substituted with —C₁₋₆alkyl, or —OC₁₋₆alkyl; wherein -L²- is —CH₂O—; or
(xi) heterocycloalkyl or -L₃-heterocycloalkyl: wherein the heterocycloalkyl is selected from the group consisting of: 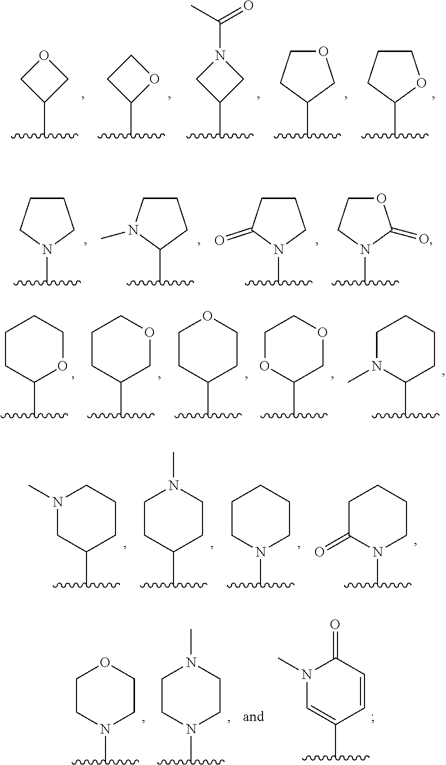
wherein each heterocycloalkyl is unsubstituted or substituted with one or two members each independently selected from: —F, —OH, —CH₂OH, and —C₁₋₆alkyl;
wherein -L₃- is —C₁₋₃alkyl-; or
(xii) 5-membered heteroaryl or -L³-(5-membered heteroaryl), wherein the 5-membered heteroaryl is selected from the group consisting of: 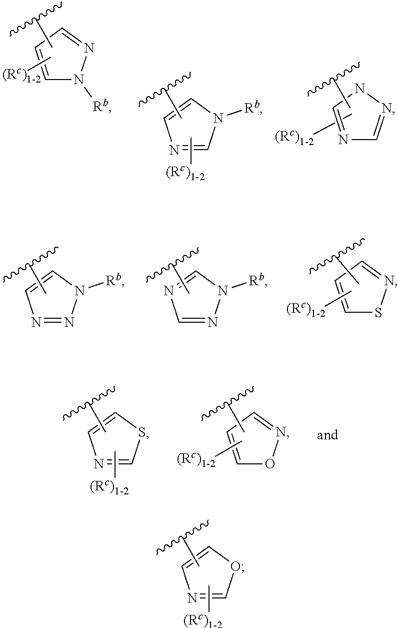
wherein R^{b} is —C₁₋₆alkyl or cyclopropyl; each R^{c} is independently selected from the group consisting of: H, —C₁₋₆alkyl, —OC₁₋₆alkyl, and cyclopropyl; wherein -L³- is —C₁₋₃alkyl; or
(xiii) 6-membered heteroaryl; wherein the 6-membered heteroaryl is selected from the group consisting of: 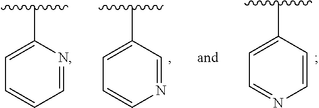
wherein each 6-membered heteroaryl is unsubstituted or substituted with one or two members each independently selected from the group consisting of: —F, —C₁₋₆alkyl, —C₁₋₃haloalkyl, and —OC₁₋₆alkyl; or
(xiv) 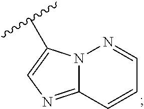 [SMILES: *c1cnc2cccnn12]
and
R² is 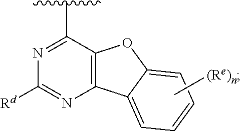
wherein
R^{d} is selected from the group consisting of: —C₁₋₆alkyl; —C₁₋₆haloalkyl; —OC₁₋₆alkyl; -cyclopropyl; -heterocycloalkyl, wherein the heterocycloalkyl is selected from the group consisting of 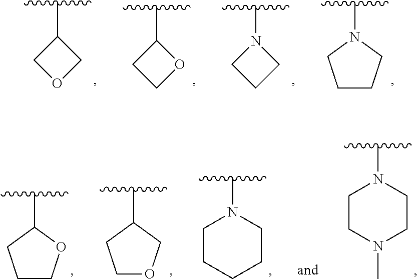
wherein each heterocycloalkyl is unsubstituted or substituted with one or two substituents each independently selected from halo, and —C₁₋₆alkyl; and 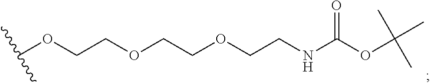 [SMILES: *OCCOCCOCCNC(=O)OC(C)(C)C]
R^{e} is selected from the group consisting of: —F, —C₁₋₆alkyl, and —C₁₋₆haloalkyl; and
n is 0, 1, or 2.

2. The compound of claim 1, or a pharmaceutically acceptable salt, wherein
X is H and \textcircled{A} is

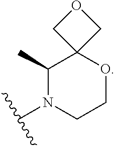 [SMILES: *N1CCOC2(COC2)[C@@H]1C]

3. The compound of claim 1, or a pharmaceutically acceptable salt thereof, wherein
R^{a} is 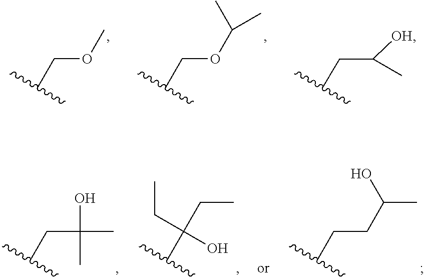
or
R^{a} is 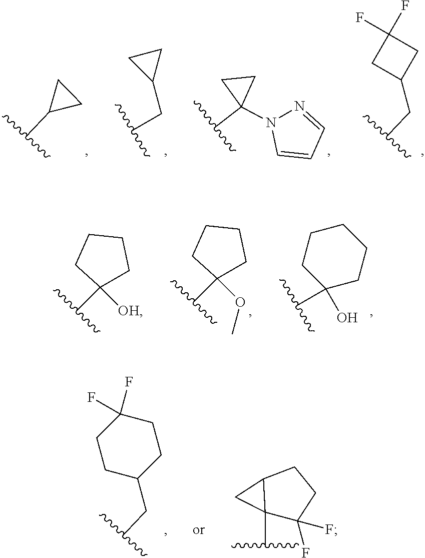
or
R^{a} is 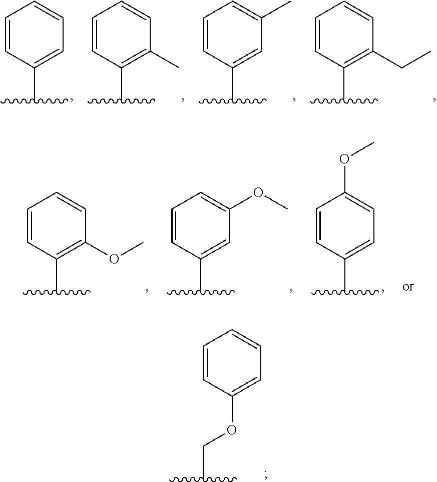
or
R^{a} is 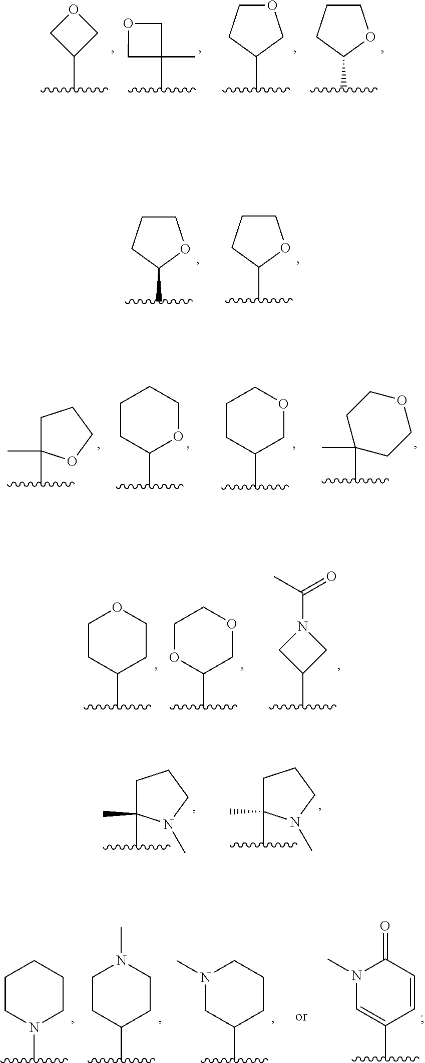
or
R^{a} is 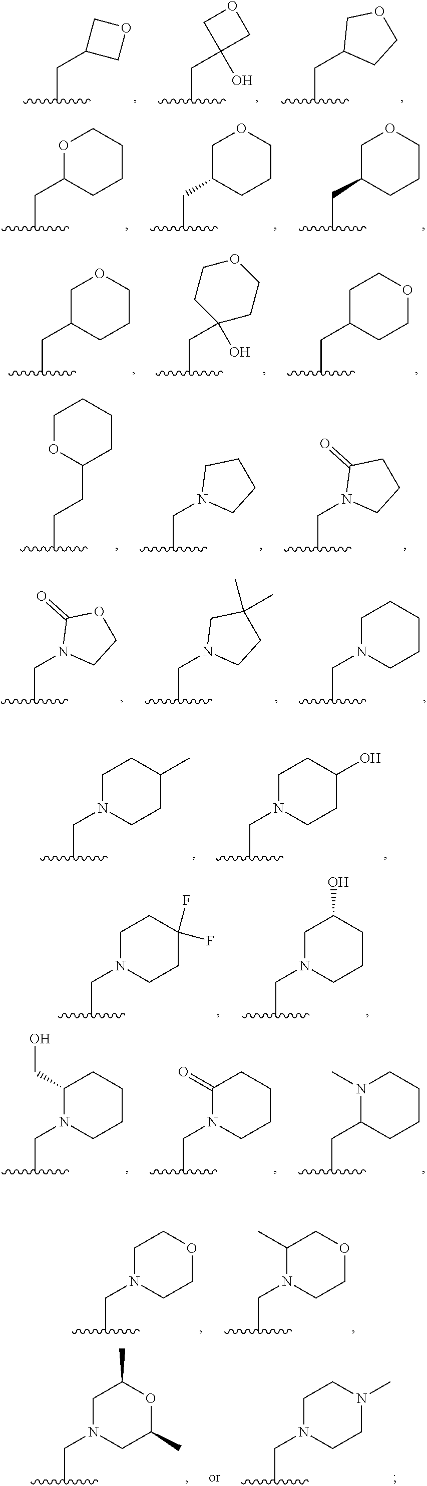
or
R^{a} is 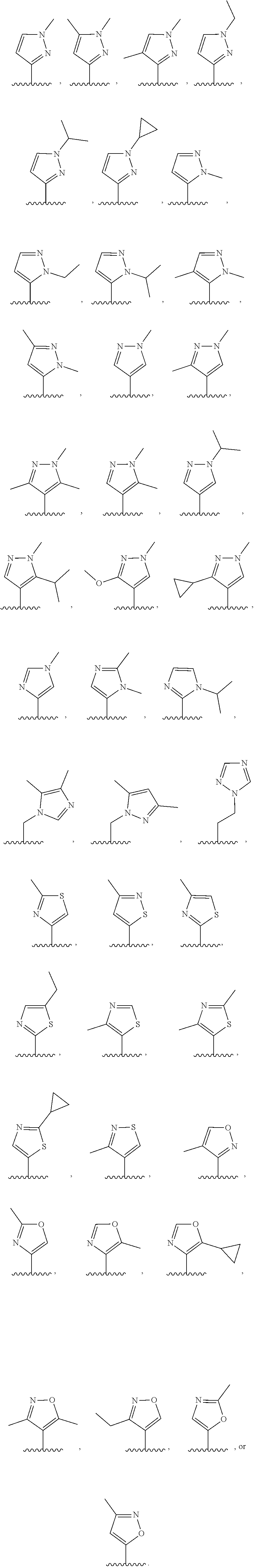
or 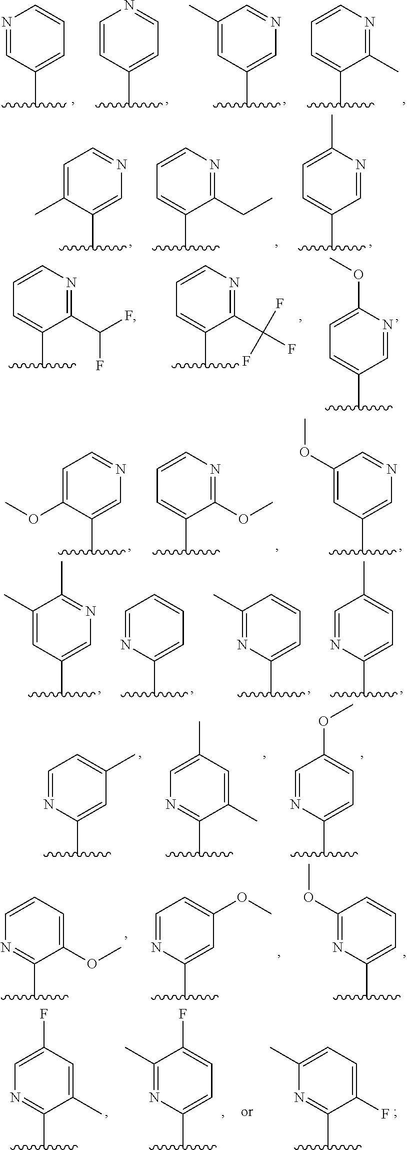
or
R^{a} is

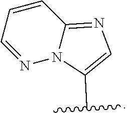 [SMILES: *c1cnc2cccnn12]

4. The compound of claim 1, or a pharmaceutically acceptable salt, wherein
R^{d} is —CH₃, —CH₂CH₃, —CH(CH₃)₂, —C(CH₃)₃, —CHF₂, —CF₃, —OCH₃, —OCH₂CH₃, —OCH(CH₃)₂, or —CH(CH₃)(CF₃); or
R^{d} is -cyclopropyl; or
R^{d} is 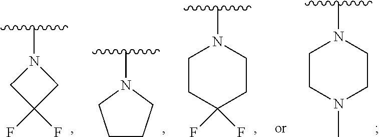
or
R^{d} is H 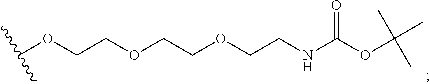 [SMILES: *OCCOCCOCCNC(=O)OC(C)(C)C]
or
R^{d} is

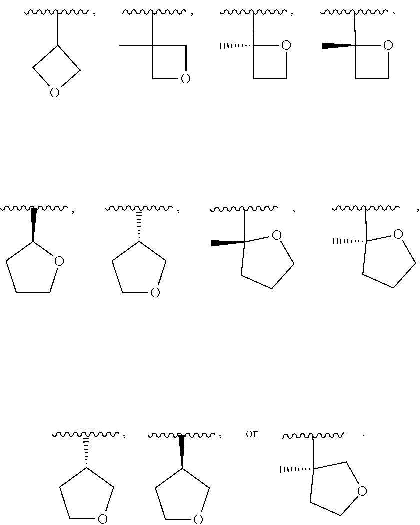

5. The compound of claim 1, or a pharmaceutically acceptable salt thereof, wherein
R² is 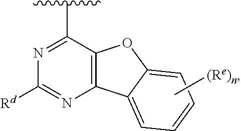
R^{e} is —F, —CH₃, or —CF, and n is 0, 1, or 2.

6. The compound of claim 1, or a pharmaceutically acceptable salt thereof, wherein
R^{d} is 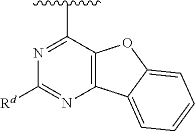
and R^{d} is —CH₃ or —CH₂CH₃.

7. A compound of Table 1, or a pharmaceutically acceptable salt thereof.

8. The compound of claim 1, or a pharmaceutically acceptable salt thereof, wherein the compound is a compound of Formula (IA): 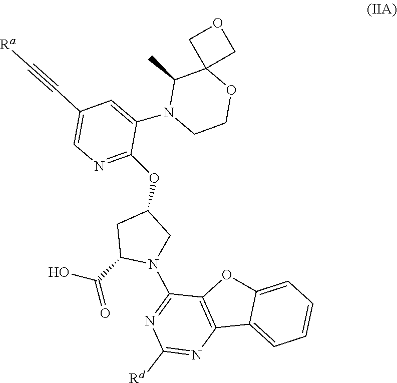
wherein
R^{a} is
(iv) phenyl, wherein the phenyl is unsubstituted or substituted with one member selected from the croup consisting of: —C₁₋₆alkyl or —OCH₃; or
(v) 5-membered heteroaryl: wherein the 5-membered heteroaryl is selected from the group consisting of: 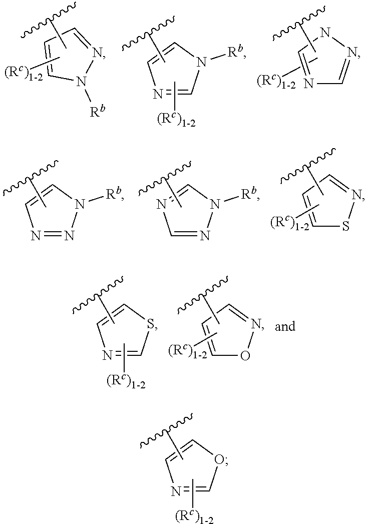
wherein R^{b} is —C₁₋₃alkyl or cyclopropyl; each R^{c} is independently selected from the group consisting of: H, —C₁₋₃alkyl, —OCH₃, and cyclopropyl; or
(vi) 6-membered heteroaryl; wherein the 6-membered heteroaryl is selected from the group consisting of: 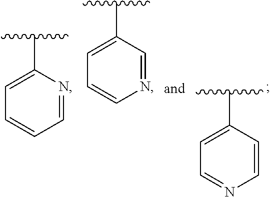
wherein each 6-membered heteroaryl is unsubstituted or substituted with one or two members each independently selected from the group consisting of: —F, —C₁₋₃alkyl, —CF₂H, —CF₃, and —OCH₃; and
R^{d} is
(v) —CH₃, —CH₂CH₃, —CH(CH₃)₂, —C(CH₃)₃, —CHF₂, —CF₃, —OCH₃, —OCH₂CH₃, —CH(CH₃)(CF₃) 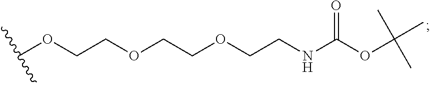 [SMILES: *OCCOCCOCCNC(=O)OC(C)(C)C]
or
(vi) -cyclopropyl; or
(vii)
(viii)

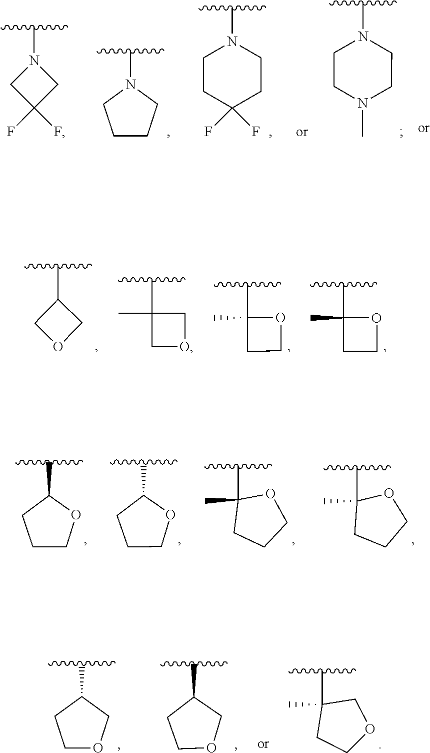

9. The compound of claim 1, or a pharmaceutically acceptable salt thereof, wherein the compound is a compound of Formula (IIB): 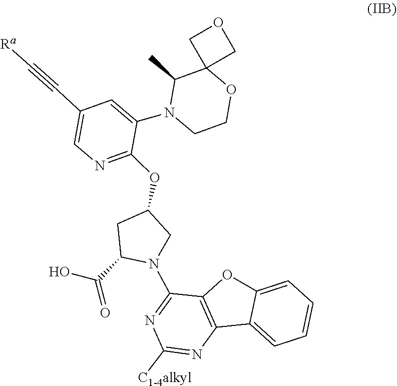
wherein
R^{a} is
(i) 5-membered heteroaryl; wherein the 5-membered heteroaryl is selected from the group consisting of: 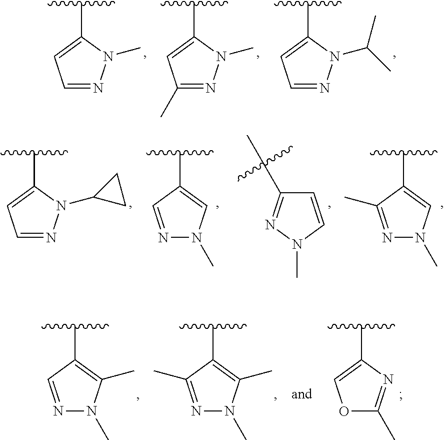
or
(ii) 6-membered heteroaryl; wherein the 6-membered heteroaryl is selected from the group consisting of:

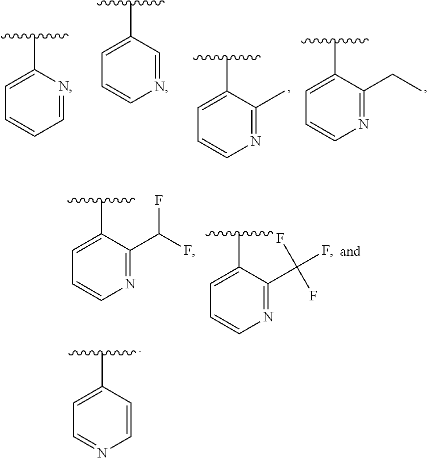

10. The compound of claim 1, or a pharmaceutically acceptable salt thereof, wherein the compound is a compound of Formula (IIC): 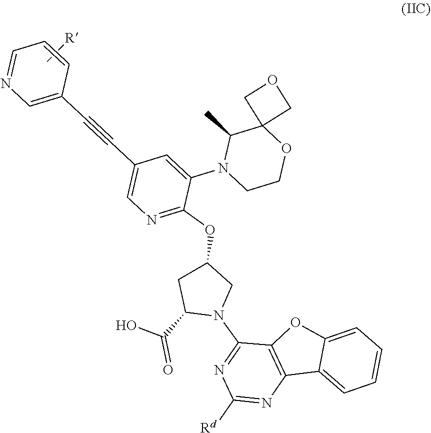
wherein R′ is —CH and R^{d} is —CH₃ or —CH₂CH₃.

11. The compound of claim 10, wherein the compound is a compound of Formula (IIC′): 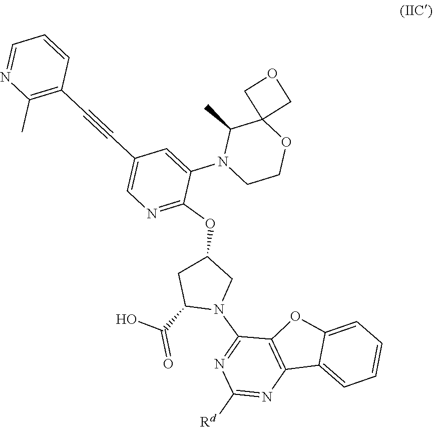
wherein R^{d} is —CH₃ or —CH₂CH₃.

12. A compound, or a pharmaceutically acceptable salt thereof, selected from the group consisting of:

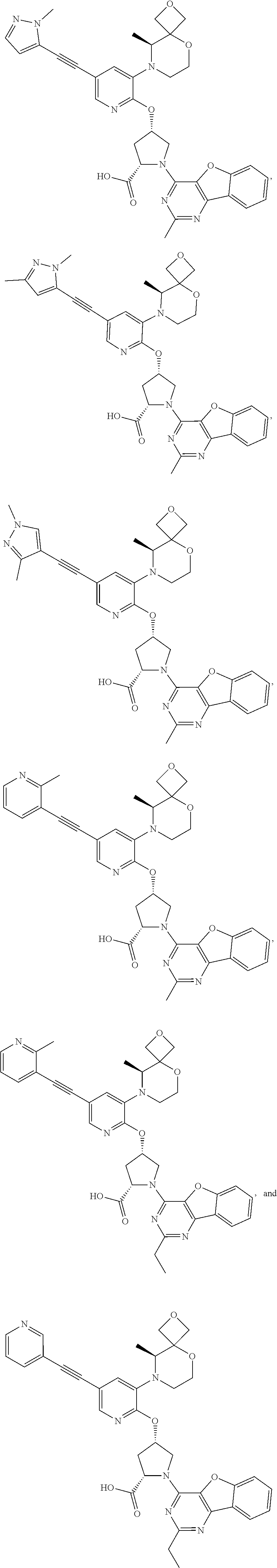

13. The compound of claim 12, which is:

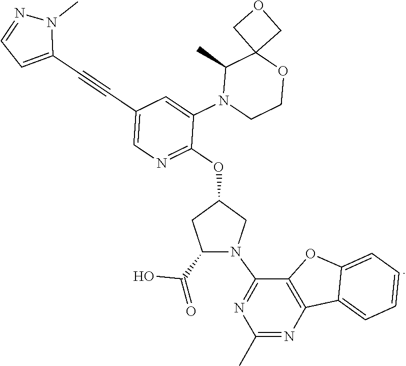 [SMILES: Cc1nc(N2C[C@@H](Oc3ncc(C#Cc4ccnn4C)cc3N3CCOC4(COC4)[C@@H]3C)C[C@H]2C(=O)O)c2oc3ccccc3c2n1]

14. The compound of claim 12, which is:

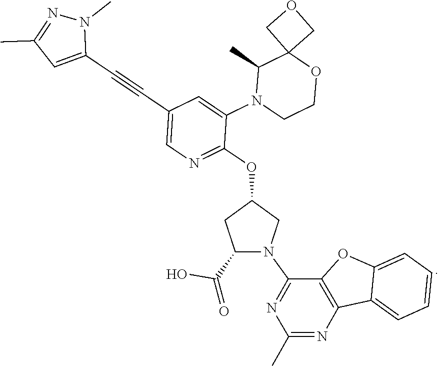 [SMILES: Cc1cc(C#Cc2cnc(O[C@H]3C[C@@H](C(=O)O)N(c4nc(C)nc5c4oc4ccccc45)C3)c(N3CCOC4(COC4)[C@@H]3C)c2)n(C)n1]

15. The compound of claim 12, which is:

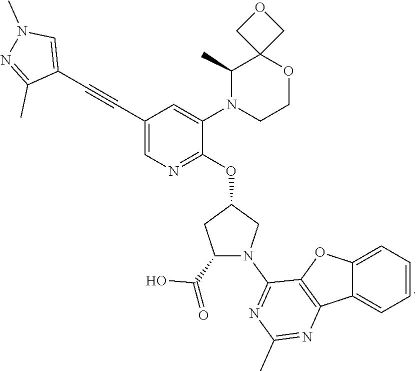 [SMILES: Cc1nc(N2C[C@@H](Oc3ncc(C#Cc4cn(C)nc4C)cc3N3CCOC4(COC4)[C@@H]3C)C[C@H]2C(=O)O)c2oc3ccccc3c2n1]

16. The compound of claim 12, which is

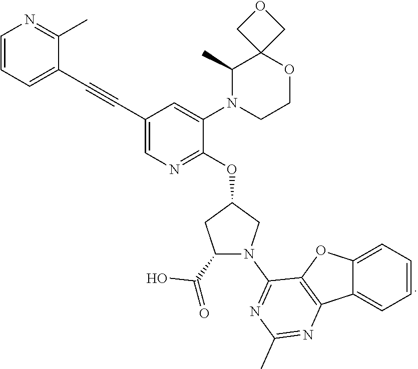 [SMILES: Cc1nc(N2C[C@@H](Oc3ncc(C#Cc4cccnc4C)cc3N3CCOC4(COC4)[C@@H]3C)C[C@H]2C(=O)O)c2oc3ccccc3c2n1]

17. The compound of claim 12, which is:

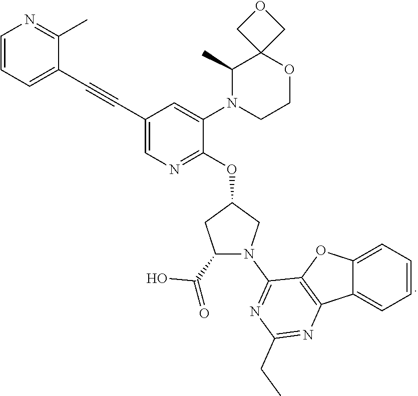 [SMILES: CCc1nc(N2C[C@@H](Oc3ncc(C#Cc4cccnc4C)cc3N3CCOC4(COC4)[C@@H]3C)C[C@H]2C(=O)O)c2oc3ccccc3c2n1]

18. The compound of claim 12, which is:

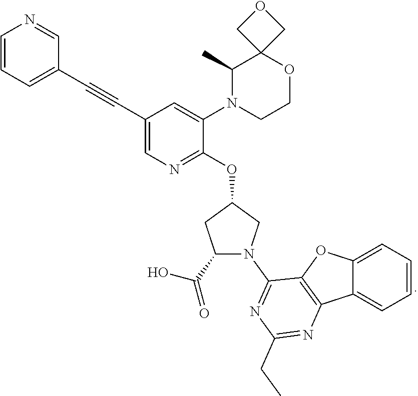 [SMILES: CCc1nc(N2C[C@@H](Oc3ncc(C#Cc4cccnc4)cc3N3CCOC4(COC4)[C@@H]3C)C[C@H]2C(=O)O)c2oc3ccccc3c2n1]

19. A pharmaceutical composition comprising a therapeutically effective amount of a compound according to claim 1, or a pharmaceutically acceptable salt thereof, and at least one pharmaceutically acceptable excipient.

20. A method of treating a subject suffering from or diagnosed with a disease, disorder, or condition mediated by the cGAS pathway, comprising administering to a subject in need of such treatment a therapeutically effective amount of a compound according to claim 1, or a pharmaceutically acceptable salt thereof.

21. The method of claim 20, wherein the cGAS mediated disease, disorder, or condition is selected from the group consisting of autoimmune disorders selected from the group consisting of: Aicardi-Goutieres Syndrome (AGS), Systemic Lupus Erythematosus (SLE), Lupus Nephritis, Scleroderma, Sjogren's Syndrome, Inflammatory Myopathies, Hidradenitis Supperativa (HS), Parkinson's Disease, Rheumatoid Arthritis, Ulcerative Colitis and Crohn's Disease.
